(19) United States
(12) Patent Application Publication
(10) Pub. No.: US 2019/0309284 A1
(43) Pub. Date: Oct. 10, 2019

(54) METHODS AND KITS FOR DYNAMIC TARGETED HYPERMUTATION

(71) Applicant: Massachusetts Institute of Technology, Cambridge, MA (US)

(72) Inventors: Matthew D. Shoulders, Cambridge, MA (US); Louis John Papa, Cambridge, MA (US); Christopher Lawrence Moore, Cambridge, MA (US)

(73) Assignee: Massachusetts Institute of Technology, Cambridge, MA (US)

(21) Appl. No.: 16/357,790

(22) Filed: Mar. 19, 2019

Related U.S. Application Data
(60) Provisional application No. 62/644,736, filed on Mar. 19, 2018.

(51) Int. Cl. C12N 15/10; C12N 15/90; C12N 9/22; C12N 9/78

(52) U.S. Cl. CPC C12N 15/102; C12N 15/907; C12N 9/22; C12N 9/78; C12Y 305/04005; C12Y 305/04007; C12Y 305/04003; C12Y 305/04001; C12Y 305/04002; C12Y 305/04006; C12Y 305/04004

(57) ABSTRACT

Disclosed herein are methodologies and kits for dynamic targeted hypermutation that harness the enzymatic activity of a polynucleic acid-binding protein fused to a nucleobase-editing enzyme to specifically target mutations across a region of interest. These methodologies and kits facilitate the rapid creation of diverse DNA libraries in vivo or in vitro.

### RELATED APPLICATION

This application claims the benefit under 35 U.S.C. § 119(e) of U.S. Provisional Application No. 62/644,736, filed on Mar. 19, 2018, and entitled “Methods and Kits for Dynamic Hypermutation,” which is incorporated herein by reference in its entirety for all purposes.

### GOVERNMENT SUPPORT

This invention was made with Government support under Grant No. GM119162 awarded by the National Institutes of Health (NIH). The Government has certain rights in this invention.

### FIELD

Disclosed herein are methodologies and kits for dynamic targeted hypermutation that harness the enzymatic activity of a polynucleic acid-binding protein fused to a nucleobase-editing enzyme to specifically target mutations across a region of interest. These methodologies and kits facilitate the rapid creation of diverse DNA libraries in vivo or in vitro.

### BACKGROUND

Mutagenesis is central to the generation of diverse target gene libraries. Previously described in vitro mutagenesis methodologies allow precise control over sites of mutation; however, they are laborious and time-consuming. Moreover, previously described methodologies directed at the generation of large, diverse libraries in vivo generally act globally on the organism (i.e., they indiscriminately alter DNA sequences in living systems, resulting in undesired off-target mutations). The off-target mutations caused by global mutagenesis result in two major drawbacks in the context of directed evolution. First, they increase the chances of false positives whereby an off-target mutation increases the fitness of an organism and enables “cheating” of the selection process. Second, they result in undesired toxicity due to the off-target mutation of critical genes. These drawbacks require users to carefully optimize global mutagenesis such that the mutation rate is maximized while cellular toxicity is minimized. The careful balance between the number of mutations and cell death constrains mutation rates, ultimately limiting library size and resulting in a lower chance of finding an improved variant and/or a less active final product of the directed evolution process.

### SUMMARY

Lab-timescale evolution relies on the generation of large mutational libraries to rapidly explore biomolecule sequence landscapes. Although numerous in vitro mutagenesis techniques are available, in vivo mutagenesis is limited (Wong et al., Comb. Chem. High Throughput Screen. 2006 May; 9(4): 271-88.). Global mutagenesis methods are capable of increasing mutation rates in vivo but unfortunately introduce extensive off-target mutations in essential and cheating genes.

In some aspects the disclosure relates to dynamic targeted hypermutation (DTH), a novel methodology for specifically targeting mutations across a gene of interest. This methodology facilitates the rapid creation of diverse DNA libraries in vivo or in vitro such that increased mutation rates are constrained to the target DNA of interest.

In some aspects the disclosure relates to nucleobase-editing fusion proteins capable of introducing nucleobase mutations in a pre-existing polynucleic acid sequence. In some embodiments, a nucleobase-editing fusion protein comprises a processive polynucleic acid-binding protein fused to a nucleobase-editing enzyme.

In some embodiments, the processive polynucleic acid-binding protein of the nucleobase-editing fusion protein comprises the amino acid sequence of an RNA polymerase, a DNA polymerase, a DNA methyltransferase, a DNA glycosylase, or a DNA helicase. In some embodiments, the processive polynucleic acid-binding protein of the nucleobase-editing fusion proteins comprises the amino acid sequence of T7 RNA polymerase or a functional variant thereof.

In some embodiments, the nucleobase-editing enzyme comprises the amino acid sequence of an Apobec protein, a TadA protein, an AMPD protein, a CDA protein, an ADAT protein, an ADAR protein, or a GDA protein. In some embodiments, the nucleobase-editing enzyme comprises the amino acid sequence of an Apobec protein. In some embodiments, the Apobec protein is rApobec1 or a functional variant thereof. In some embodiments, the nucleobase-editing enzyme comprises the amino acid sequence of a TadA protein. In some embodiments, the TadA protein is E. coli TadA comprising an A106V mutation and/or a D108N mutation or a protein homolog comprising a homologous mutation(s).

In some aspects, the disclosure relates to methods of performing dynamic targeted hypermutation. In some embodiments, the method comprises contacting at least one polynucleic acid with at least one non-naturally occurring nucleobase-editing fusion protein, wherein: (a) each of the at least one non-naturally occurring nucleobase-editing fusion proteins comprises a processive polynucleic acid-binding protein fused to a nucleobase-editing enzyme; (b) each of the at least one polynucleic acid comprises a target region; and (c) the contacting of the at least one polynucleic acid with the at least one non-naturally occurring nucleobase-editing fusion protein generates mutations at a rate exceeding background mutation rates only in the target region of the at least one polynucleic acid of (b), wherein the background mutation rate of the at least one polynucleic acid of (b) is determined in the absence of the non-naturally occurring nucleobase-editing fusion protein.

In some embodiments, the processive polynucleic acid-binding protein of at least one of the at least one non-naturally occurring nucleobase-editing fusion proteins comprises the amino acid sequence of an RNA polymerase, a DNA polymerase, a DNA methyltransferase, a DNA glycosylase, or a DNA helicase. In some embodiments, the processive polynucleic acid-binding protein of at least one of the at least one non-naturally occurring nucleobase-editing fusion proteins comprises the amino acid sequence of T7 RNA polymerase or a functional variant thereof.

In some embodiments, the nucleobase-editing enzyme of at least one of the at least one non-naturally occurring nucleobase-editing fusion proteins comprises the amino acid sequence of an Apobec protein, a TadA protein, an AMPD protein, a CDA protein, an ADAT protein, an ADAR protein, or a GDA protein. In some embodiments, the nucleobase-editing enzyme of at least one of the at least one non-naturally occurring nucleobase-editing fusion proteins comprises the amino acid sequence of an Apobec protein. In some embodiments, the Apobec protein is rApobec1 or a functional variant thereof. In some embodiments, the nucleobase-editing enzyme comprises the amino acid sequence of a TadA protein. In some embodiments, the TadA protein is E. coli TadA comprising an A106V mutation and/or a D108N mutation or a protein homolog comprising a homologous mutation(s).

In some embodiments, each of the at least one polynucleic acid comprises, from 5′ to 3′: a promoter region that is bound by at least one of the at least one non-naturally occurring nucleobase-editing fusion proteins in a sequence-specific manner; the target region; and a terminator region comprising a terminator array.

In some embodiments, the terminator array comprises four or more terminators, optionally four or more T7 UUCG terminators.

In some embodiments, the promoter region of at least one of the at least one polynucleic acids comprises the sequence of SEQ ID NO: 21, SEQ ID NO: 22, and/or SEQ ID NO: 23.

In some embodiments, the contacting of the at least one polynucleic acid with the at least one non-naturally occurring nucleobase-editing fusion protein occurs in a living cell.

In some embodiments, at least one of the at least non-naturally occurring nucleobase-editing fusion proteins is encoded for on a plasmid, wherein the plasmid has copy number of less than 10. In some embodiments, at least one of the at least one non-naturally occurring nucleobase-editing fusion proteins is conditionally expressed in the living cell.

In some embodiments, the living cell contains a modified genome comprising: (a) an integration of a polynucleic acid sequence encoding for and driving the expression of at least one non-naturally occurring nucleobase-editing fusion protein; and/or (b) an integration of a polynucleic sequence comprising, from 5′ to 3′: a promoter region that is bound by at least one of the at least one non-naturally occurring nucleobase-editing fusion proteins in a sequence-specific manner; the target region; and a terminator region comprising a terminator array.

In some embodiments, the living cell contains a modified genome and a plasmid that facilitates expression of a T7 inhibitor, wherein the modified genome of the living cell comprises: (a) an integration of a polynucleic acid sequence encoding for and driving the expression of the non-naturally occurring nucleobase-editing fusion protein, wherein the sequence driving the expression of the fusion protein comprises a sequence bound by LacI repressor that inhibits transcription of the fusion protein when LacI is bound; and/or (b) a deletion of genomic sequence encoding for uracil deglycosylase. In some embodiments, the T7 inhibitor is T7 lysozyme.

In some embodiments, the living cell is treated to increase the expression and/or activity of the uracil deglycosylase inhibitor, ugi.

In some aspects, the disclosure relates to kits for performing dynamic targeted hypermutation. In some embodiments, a kit comprises: (a) a polypeptide comprising the amino acid sequence of a non-naturally occurring nucleobase-editing fusion protein comprising a processive polynucleic acid-binding protein fused to a nucleobase-editing enzyme; and (b) a polynucleic acid sequence comprising, from 5′ to 3′: a promoter region that is bound by the non-naturally occurring nucleobase-editing fusion protein of (a) in a sequence-specific manner; a cloning site; and a terminator region comprising a terminator array.

In some embodiments, a kit comprises: (a) a polynucleic acid sequence encoding for and driving the expression of a non-naturally occurring nucleobase-editing fusion protein comprising a processive polynucleic acid-binding protein fused to a nucleobase-editing enzyme; and (b) a polynucleic acid sequence comprising, from 5′ to 3′: a promoter region that is bound by the non-naturally occurring nucleobase-editing fusion protein of (a) in a sequence-specific manner; a cloning site; and a terminator region comprising a terminator array.

In some embodiments, the processive polynucleic acid-binding protein of the non-naturally occurring nucleobase-editing fusion protein comprises the amino acid sequence of an RNA polymerase, a DNA polymerase, a DNA methyltransferase, a DNA glycosylase, or a DNA helicase. In some embodiments, the processive polynucleic acid-binding protein of the non-naturally occurring nucleobase-editing fusion proteins comprises the amino acid sequence of T7 RNA polymerase or a functional variant thereof.

In some embodiments, the nucleobase-editing enzyme of the nucleobase-editing fusion protein comprises the amino acid sequence of an Apobec protein, a TadA protein, an AMPD protein, a CDA protein, an ADAT protein, an ADAR protein, or a GDA protein. In some embodiments, the nucleobase-editing enzyme of the nucleobase-editing fusion protein comprises the amino acid sequence of an Apobec protein. In some embodiments, the Apobec protein is rApobec1 or a functional variant thereof. In some embodiments, the Apobec protein is rApobec1 or a functional variant thereof. In some embodiments, the nucleobase-editing enzyme comprises the amino acid sequence of a TadA protein. In some embodiments, the TadA protein is E. coli TadA comprising an A106V mutation and/or a D108N mutation or a protein homolog comprising a homologous mutation(s).

In some embodiments, the terminator array comprises four or more terminators, optionally four or more T7 UUCG terminators.

In some embodiments, the promoter region comprises the sequence of SEQ ID NO: 21, SEQ ID NO: 22, and/or SEQ ID NO: 23.

These and other aspects of the invention are further described below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1G. Measurement of targeted mutagenesis capabilities of MutaT7 (an embodiment of a rApo1 fused to T7 RNA polymerase). FIG. 1A. Schematic demonstrating the differences between global and targeted mutagenesis. FIG. 1B. Diagram depicting the processive cycle through which MutaT7 performs targeted mutagenesis. FIG. 1C. Schematic of a drug resistance start codon reversion reporter assay for measuring extent of mutational targeting to specific loci of DNA. The first gene (Kan^{R}) reports on-target activity, while the second gene (Tet^{R}) reports activity downstream of a DNA spacer element. FIG. 1D. Codon reversion reporter assay data for different combinations of mutagen and reporter elements after 24 hr of culturing. Kanamycin and tetracycline drug resistance frequencies are represented as solid and candy-stripe bars, respectively. FIG. 1E. Extent of off-target mutagenesis assessed by rifampicin drug resistance frequency. FIG. 1F. Cell viability data for populations of cells following expression with different mutagenic constructs (solid bars) or treatment with chemical mutagen (candy-stripe bar). FIG. 1G. Total level of kanamycin resistant colonies following expression with different genetically encoded mutagens for 24 hr. FIGS. 1D-1G. All data reported is the average of biological replicates (n=3). Error bars represent SEM. Statistically significant comparisons are shown with stars (t-test), while p-values of notable non-significant values are shown as well.

FIGS. 2A-2F. Sequencing-based assessment of mutational targeting by MutaT7 during continuous culturing. FIG. 2A. Diagram of reporter construct and continuous culture experiment to assess mutation accumulation under drift conditions. FIG. 2B. Schematic and representation of mutations observed by Sanger sequencing 96 clones in respective cell populations following 15 days of continuous growth without selection pressure. FIG. 2C. Visual representation of on-target and off-target mutations identified by sequencing episomes propagated in the presence of targeted (MutaT7) and global (MP6) mutagens. Normalized mutation frequency (number of mutations observed divided by number of kb of DNA sequenced in associated regions) and on:target to off:target mutation ratio are shown to the right. FIG. 2D. A diagram of continuous culture conditions used to propagate a dual promoter episome in cells expressing mutaT7, along with details for downstream Sanger sequencing analysis work flow. FIG. 2E. A graph of mutations observed by Sanger sequencing target gene from 10 clones at different time points (triangles for total mutations, circles for C to T transitions, squares for G to A transitions). FIG. 2F. Box and whisker plot of mutations from FIG. 2C, where each dot represents a single clone. Mean is represented by horizontal line and fences extend down to minimum and up to maximum.

FIGS. 3A-3B. The deleterious effects of global mutagenesis. FIG. 3A. Global mutagenesis indiscriminately introduces mutations across an organism's entire genome, introducing mutations in target genes and other genes such as essential genes. Attempts to increase the global mutagenesis rate and thus library diversity lead to decreased cell viability due to off-target mutations in essential genes. Targeted mutagenesis allows for a high mutagenesis rate that does not decrease cell viability by minimizing off-target mutations in essential genes. FIG. 3B. Global mutagenesis can also cause off-target mutations in genes that allow an organism to cheat the selection, thus causing a certain rate of false positives. Targeted mutagenesis minimizes false positives by preventing off-target mutations in genes that allow the organism to cheat the selection.

FIGS. 4A-4B. Multiple terminators required to prevent downstream mutations. FIG. 4A. Reporter plasmids were used that have a kanamycin resistance gene that lacks a start codon downstream from a T7 promoter. The kanamycin resistance gene is followed by a variable number of T7 transcriptional terminators and a tetracycline resistance gene that lacks a start codon. Mutations that revert the ACG codon to an ATG start codon in the kanamycin or tetracycline resistance gene lead to kanamycin or tetracycline resistant colonies, respectively. FIG. 4B. After growing the reporter plasmid in the MutaT7 strain for 24 hours, the frequency of kanamycin resistant colonies is relatively constant regardless of the number of terminators between the kanamycin and tetracycline resistance genes. The frequency of tetracycline resistant colonies decreases as more terminators are introduced between the kanamycin and tetracycline genes. After about 4 terminators, the tetracycline resistance frequency is at background resistance levels (as determined by a drApo1−T7 strain negative control). This suggests that an array of terminators is able to stop T7 transcription and thus MutaT7 mutations downstream from the terminator array.

FIG. 5. Mutation assay workflow. The mutation assay workflow is shown. Glycerol stocks of each sample are streaked on LB agar with appropriate antibiotics and grown at 37° C. for 24 hours. Then, single colonies are picked in triplicate and grown in LB with appropriate antibiotics and inducers of mutagenesis at 37° C. for 24 hours. Then, 1 mL aliquots of each culture are pelleted and resuspended in LB to remove antibiotics and inducers. The resuspension is then plated at various dilutions on plates with various antibiotics to test the mutation rate and a metabolic dye, tetrazolium chloride, for contrast during imaging. After growing at 37° C. for 48 hours, the plates are imaged on a document scanner at 400 d.p.i. and colonies are counted using the OpenCFU (3.9.0) software (Geissmann et al., PLoS One. 2013; 8(2): e54072).

FIG. 6. Optimizing antibiotic concentrations for the mutation assay. At concentrations of less than 200 μg/mL, small colonies (black arrows) appeared on LB+kanamycin+tetrazolium chloride plates with DH10B carrying the reporter plasmid. The small colonies could be present due to a low level of expression of the kanamycin resistance gene through translation initiation from the ACG start codon (Hecht et al., Nucleic Acids Res. 2017 Apr. 20; 45(7): 3615-26). On plates with 200 μg/mL kanamycin, the small colonies on the DH10B plate do not appear after 48 hours. The number of colonies on plates of MutaT7 cells with the reporter plasmid were similar between plates with 150 μg/mL and 200 μg/mL kanamycin.

FIGS. 7A-7D. Additional mutation assay data. FIG. 7A. Additional kanamycin, tetracycline and FIG. 7B rifampicin resistance frequency data for Δung and drApo1 negative control strains with various reporter plasmids. FIG. 7C. Fosfomycin resistance frequency data shows a high mutagenesis rate only in the presence of MP6, suggesting that neither MutaT7 nor the negative controls mutagenize the E. coli genome appreciably. FIG. 7D. Additional ampicillin resistance frequency data suggests that neither the Δung nor the drApo1 negative control strains suffer from low cell viability.

FIG. 8. Kanamycin and tetracycline resistance frequencies without the reporter plasmids. To ensure that the kanamycin and tetracycline resistant colonies in the mutation assay are due to mutations in the reporter plasmid and not mutations in the genome, the MP6 strain and MutaT7 strain were grown for 24 hours without reporter plasmids in LB with 100 μg/mL streptomycin and 25 mM arabinose (with 10 μg/mL chloramphenicol for the MP6 strain). After washing once with LB, 50 μL of each culture was plated on LB with 50 μg/mL tetrazolium chloride and 30 μg/mL kanamycin, 200 μg/mL kanamycin or 20 μg/mL tetracycline. The lack of colonies on plates with 200 μg/mL kanamycin or 20 μg/mL tetracycline suggests that almost all the colonies in the mutation assay at these concentrations are due to mutations in the reporter plasmid. However, at lower kanamycin concentrations, kanamycin resistance colonies appear in the MP6 strain, suggesting that mutations are occurring in the genome of MP6 that can confer moderate kanamycin resistance.

FIGS. 9A-9B. Promoter design. FIG. 9A. The P_{AllacO-1} promoter has been engineered to have minimal leaky expression when repressed with lacI (Camsund et al., J. Biol. Eng. 2014 Jan. 27; 8(1): 4). The BBa_J23114 promoter (SEQ ID NO: 16) from the Anderson Collection (parts.igem.org/Promoters/Catalog/Anderson) has been shown to have about a tenth of the strength of the σ⁷⁰ consensus binding sites. With the intention of obtaining a weak, strongly repressed promoter, the σ⁷⁰ binding sites of BBa_J23114 (SEQ ID NO: 16) were grafted onto P_{AllacO-1} (SEQ ID NO: 93) to yield P_{AllacO-Tenth} (SEQ ID NO: 24) (changes include TTGAC [SEQ ID NO: 25] to TTTAT [SEQ ID NO: 26] at −35, GATACT [SEQ ID NO: 27] to TACAAT [SEQ ID NO: 28] at −10). FIG. 9B. In order to increase the expression of lacI from the DH10B genome, the endogenous P_{lacI} promoter (SEQ ID NO: 94) was replaced with the strong, constitutive P_{tac} promoter (SEQ ID NO: 95) to yield the P_{lacI}<>P_{tac} promoter (SEQ ID NO: 96) (Glascock C. B. and Weickert M. J., Gene. 1998 Nov. 26; 223(1-2): 221-31).

FIGS. 10A-10C. Catalytically dead rApo1. FIG. 10A. A clustalw (Larkin et al., Bioinformatics. 2007 Nov. 1; 23(21): 2947-48) alignment of rApo1 (SEQ ID NO: 97) and another cytidine deaminase, human activation-induced cytidine deaminase (hAID) (SEQ ID NO: 98), is shown. Highlighted are aligned glutamate residues that have been shown to be critical for rApo1 activity (E63) (Navaratnam et al., Cell. 1995 Apr. 21; 81(2): 187-95) and hAID activity (E58) (Ma et al., Nat. Methods. 2016 December; 13(12): 1029-35). FIG. 10B. A crystal structure of a catalytically dead E58A mutant of hAID (PDB: 5W0U) is shown with dCMP in the active site. The position of E58 is shown based off of an alignment between 5W0U and a crystal structure of wild-type hAID with an empty active site (PDB: 5W0Z) (Qiao et al., Mol. Cell. 2017 Aug. 3; 67(3): 361-73). E58 is positioned closely to the dCMP substrate. FIG. 10C. A proposed catalytic mechanism of hAID cytidine deamination (Chaudhuri J. and Alt F. W., Nat. Rev. Immunol. 2004 July; 4(7): 541-52) based on studies with the E. coli cytidine deaminase (Betts et al., J. Mol. Biol. 1994 Jan. 14; 235(2): 635-56) is shown. The role of E58 in proton shuttling is shown. The critical E63 residue in rApo1 likely plays a similar role.

FIGS. 11A-11B. Inducible expression of MutaT7. FIG. 11A. Schematic of inducible constructs demonstrating the expected outcomes for populations of cells following 24 hours of culturing while expressing constructs that are non-mutagenic (drApo1−T7), targeted (MutaT7), or globally mutagenic (rApo1). FIG. 11B. Data for kanamycin drug resistance frequencies in response to increasing levels of IPTG treatment for 24 hours. Kanamycin reports on-target mutagenesis, and data reported is the average of biological replicates (n=3). Error bars represent SEM.

FIGS. 12A-12B. FIG. 12A. Schematic illustrating global versus targeted mutagenesis. FIG. 12B. The MutaT7 construct and the targeted mutagenesis cycle.

FIGS. 13A-13E. FIG. 13A. Drug resistance start codon reversion reporter assay for measuring extent of mutagenesis at specific DNA loci. FIG. 13B. Codon reversion reporter assay data for combinations of mutagen and reporter plasmids. Mutagens include deactivated rApo1 fused to T7 RNA polymerase (drApo1−T7; negative control), unfused rApo1 (rApo1), targeted mutagen (MutaT7), and global mutagen (MP6). FIG. 13C. Extent of off-target mutagenesis assessed by rifampicin resistance assay for populations carrying the codon reversion reporter plasmid with a terminator array in FIG. 13B (EMS=ethyl methanesulfonate). FIG. 13D. Viability data for cell populations in FIG. 13B, along with drApo1−T7 populations treated with EMS. FIG. 13E. Total number of kanamycin resistant colonies for populations in FIG. 13B. Values represent mean of independent experiments (n=3); error bars represent s.e.m.; statistical significance was evaluated by a Student's t-test: *p<0.05, **p<0.01 and ***p<0.001; notable non-significant p-values shown.

FIGS. 14A-14C. FIG. 14A. Reporter construct and continuous culture experiment to assess mutation accumulation under drift conditions. FIG. 14B. On-target (oval) and off-target (x) mutations identified by sequencing episomes propagated in the presence of targeted (MutaT7) and global (MP6) mutagens. FIG. 14C. Normalized mutation frequency (number of mutations observed divided by kb of DNA sequenced in associated regions) for data in FIG. 14B.

FIGS. 15A-15B. MutaT7 maintains a high level of activity and processivity. FIG. 15A. A general diagram of the lacZα reporter plasmid C1E, which has both a “near” and “far” T7 promoter. The rest of the lacZα reporter plasmids are missing one or both of these T7 promoters, or have a strong, constitutive P_{tac} promoter in place of the “near” T7 promoter. The genome of human adenovirus type 5 serves as the intervening DNA between the “near” and “far” promoters. FIG. 15B. LacZα activity measured via oNPG cleavage. ung⁺ served as a negative control that lacks the T7 RNA polymerase. “drApo1+T7 ung⁺” served as a positive control in which deactivated rApo1 and active T7 are expressed as separate proteins. Various reporters were used with different locations of targeted (T7) promoters and constitutive (P_{tac}) promoters, as indicated by the key on the x-axis. “LB Only” was a negative control in which LB with no cells was added to the assay mixture.

FIGS. 16A-16B. Promoter design. FIG. 16A. The P_{AllacO-1} promoter has been engineered to have minimal leaky expression when repressed with lacI (Camsund et al., J. Biol. Eng. 2014 Jan. 27; 8(1): 4). The BBa_J23114 promoter (SEQ ID NO: 16) from the Anderson Collection (Available: parts.igem.org/Promoters/Catalog/Anderson) has been shown to have about 1/10 of the strength of the σ⁷⁰ consensus binding sites. With the intention of obtaining a weak, strongly repressed promoter, the σ⁷⁰ binding sites of BBa_J23114 were grafted onto P_{AllacO-1} (SEQ ID NO: 93) to yield P_{AllacO-Tenth} (SEQ ID NO: 24) (changes include TTGAC [SEQ ID NO: 25] to TTTAT [SEQ ID NO: 26] at −35, GATACT [SEQ ID NO: 27] to TACAAT [SEQ ID NO: 28] at −10). FIG. 16B. In order to increase the expression of lacI from the DH10B genome, the endogenous P_{lacI} promoter (SEQ ID NO: 94) was replaced with the strong, constitutive P_{tac} promoter (SEQ ID NO: 95) to yield the P_{lacI}<>P_{tac} promoter (SEQ ID NO: 96) (Glascock C. B. and Weickert M. J., Gene. 1998 Nov. 26; 223(1-2): 221-31).

FIG. 17. Mutation assay workflow. The mutation assay workflow is shown. Glycerol stocks of each sample were streaked on LB agar with appropriate antibiotics and grown at 37° C. for 24 h to obtain clones. Single colonies were picked in triplicate and grown in LB with appropriate antibiotics and inducers of mutagenesis at 37° C. for 24 h to accumulate mutations. 1 mL aliquots of each culture were pelleted and resuspended in LB to remove antibiotics and inducers. The resuspension was plated at various dilutions on plates with various antibiotics to analyze the mutation rates and cell viability. The plates also contained a metabolic dye, tetrazolium chloride, for contrast during imaging. After incubating at 37° C. for 48 h, the plates were imaged on a document scanner at 400 d.p.i. and colonies were counted using the OpenCFU (3.9.0) software (Geissmann, PLoS One. 2013; 8(2): e54072).

FIG. 18. Optimizing antibiotic concentrations for mutation assays. At concentrations of less than 200 μg/mL, small colonies (black arrows) appeared on LB+kanamycin+tetrazolium chloride plates with DH10B carrying the reporter plasmid. The small colonies theoretically may have been present owing to a very low level of expression of the kanamycin resistance gene through translation initiation from the ACG start codon (Hecht et al., Nucleic Acids Res. 2017 Apr. 20; 45(7): 3615-26). On plates with 200 μg/mL kanamycin, the small colonies on the DH10B plate did not appear even after 48 h. The number of colonies on plates of MutaT7 cells (TABLE 8) with the reporter plasmid were similar between plates with 150 μg/mL and 200 μg/mL kanamycin. At a concentration of 20 μg/mL tetracycline, no colonies appeared on LB+tetracycline+tetrazolium chloride plates with DH10B cells carrying the reporter plasmid, while many colonies appeared in MutaT7 cells carrying the reporter plasmid.

FIGS. 19A-19D. Additional mutation assay data with negative control strains and fosfomycin resistance data. FIG. 19A. Kanamycin and tetracycline resistance frequency data for Δung and drApo1 negative control strains (TABLE 8) with various reporter plasmids suggest that neither strain mutagenizes the reporter plasmid appreciably. Experiment performed as in FIG. 13B with the indicated strains. FIG. 19B. Rifampicin resistance frequency data for Δung and drApo1 negative control strains with various reporter plasmids suggest that neither strain mutagenizes the E. coli genome appreciably. Experiment performed as in FIG. 13C with the indicated strains. FIG. 19C. Fosfomycin resistance frequency data show a high mutagenesis rate only in the presence of MP6, suggesting that neither MutaT7 (TABLE 8) nor the negative controls mutagenize the E. coli genome appreciably. Experiment performed as in FIG. 13C except cells were plated on LB-agar with 100 μg/mL fosfomycin and 50 μg/mL tetrazolium chloride. FIG. 19D. Ampicillin resistance frequency data suggest that neither the Δung nor drApo1 negative control strains suffer from low cell viability. Experiment performed as in FIG. 13D with the indicated strains.

FIG. 20. Multiple T7 terminators prevent downstream mutations. After growing the reporter plasmid in the MutaT7 strain (TABLE 8) for 24 h, the frequency of kanamycin resistant mutant colonies was relatively constant regardless of the number of terminators between the kanamycin and tetracycline resistance genes. The frequency of tetracycline resistant colonies decreased as more T7 terminators were introduced. After four T7 terminators were added, the tetracycline resistance frequency was restored to background levels (as evaluated using a drApo1−T7 strain as a negative control; TABLE 8).

FIGS. 21A-21B. Directed evolution of folA using MutaT7 results in a lower false positive frequency than is obtained using a global mutagen. FIG. 21A. Schematic of a directed evolution experiment on folA (promoter and protein coding sequence of dihydrofolate reductase from E. coli) designed to measure the frequency of true and false positives following mutagenesis and selection with trimethoprim (TMP). Clones propagating an episome with wild-type folA downstream of a T7 promoter were mutagenized with MutaT7 or a global mutagen (MP6) in the absence of selection pressure. Selection on LB-agar plates with TMP enabled isolation of TMP-resistant colonies. Subsequent amplification and Sanger sequencing of episomal folA genes is used to assess the frequency of true positives (drug-resistant mutations in episomal folA) and false positives (drug-resistant mutations somewhere else in genome). FIG. 21B. Summary of bacterial growth curve data measuring extent of TMP resistance in evolved isolates. Growth rates in response to increasing concentrations of TMP were determined for a representative isolate from each biological replicate along with a positive control (episomal folA, but with a strong promoter instead of wild-type promoter) and a negative control (drApo−T7 with episomal folA). After determining maximal growth rate within each sample, growth rates were normalized to the highest rate within each sample series, yielding the relative growth rate (y-axis) at each TMP concentration (x-axis).

FIG. 22. Sanger sequencing reveals mutations throughout the target region. Schematic and representation of mutations observed by Sanger sequencing 96 clones in the indicated cell populations following 15 d of continuous growth in the absence of selection pressure.

FIGS. 23A-23B. MutaT7 introduced mutations throughout the rpsL gene. FIG. 23A. Schematic of streptomycin resistance counter-selection assay, which is designed to enrich for mutations that nullify streptomycin sensitivity. Such sensitivity is initially conferred by a streptomycin-sensitive allele of rpsL downstream of a T7 promoter on a reporter plasmid. FIG. 23B. The position of various mutations throughout the T7 promoter+rpsL reporter plasmid determined by Sanger sequencing of 48 streptomycin resistant mutants from the MP6 strain and 42 streptomycin resistant mutants from the MutaT7 strain.

FIGS. 24A-24C. Dual T7 promoters introduce mutations in both strands. FIG. 24A. Diagram of continuous culture conditions used to propagate a dual promoter episome in cells expressing MutaT7, along with details for downstream Sanger sequencing analysis. FIG. 24B. Graphic of mutations observed by Sanger sequencing a target gene between dual opposing T7 promoters from clones harvested at different time points (triangles for total mutations, circles for C to T transitions, and squares for G to A transitions). FIG. 24C. Box and whisker plot of mutations from FIG. 24B, where each dot represents the number of mutations found in each clone. Mean number of mutations at each time point is represented by horizontal line.

FIGS. 25A-25C. Ugi expression increases mutagenesis by inhibiting dU to dC repair. FIG. 25A. Kanamycin resistance frequency data for the ugi rApo1 and ugi drApo1−T7 negative control strains and the ugi MutaT7 mutagenic strain (TABLE 8) with various reporter plasmids show that the ugi protein can increase mutagenesis when expression of ugi and MutaT7 from the P_{AllacO-Tenth} promoter is induced with IPTG. FIG. 25B. Tetracycline resistance frequency data for the same experiment performed in FIG. 25A. FIG. 25C. Cell viability as determined by the number of ampicillin resistance colonies for the same experiment performed in FIG. 25A.

FIGS. 26A-26D. FIG. 26A. Schematic of a drug resistance premature stop codon reversion reporter assay for measuring extent of mutational targeting via mutations in the Kan^{R} or Tet^{R} genes. FIG. 26B. Premature stop codon reversion frequencies with different mutagenic constructs. N.D. means mutants were not detected. FIG. 26C. Global off-target mutagenesis assessed by rifampicin resistance frequencies. “Not Collected” means that the rifampicin resistance was not measured for these samples. FIG. 26D. Cell viability measured by colony forming units (CFU) on plates with ampicillin and streptomycin.

### DETAILED DESCRIPTION

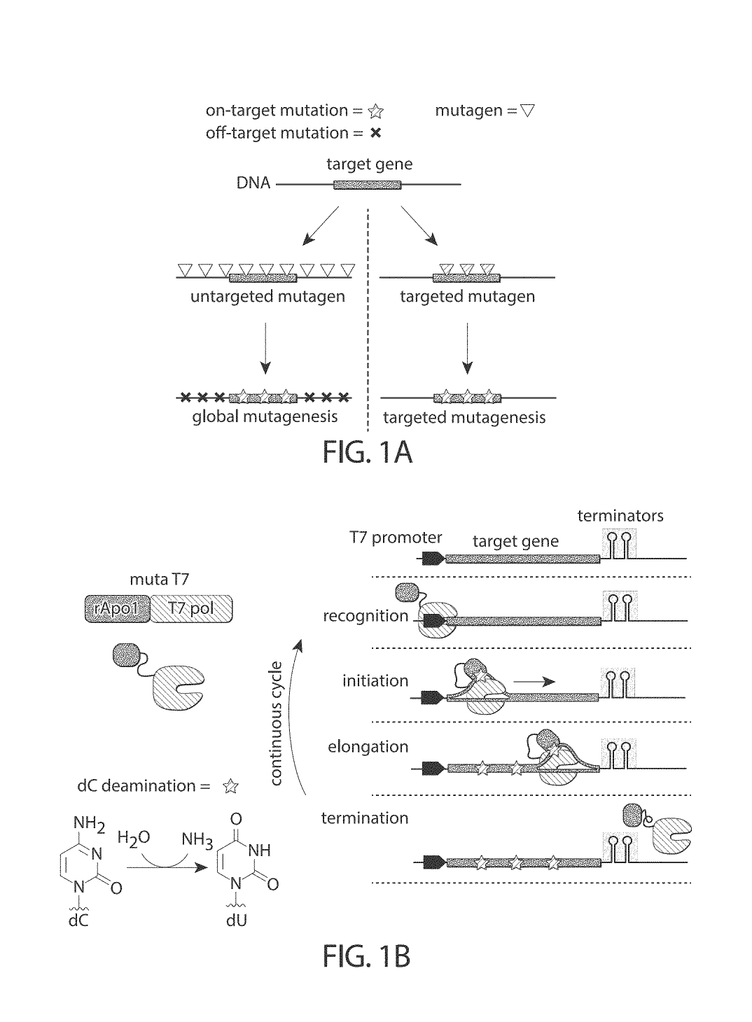

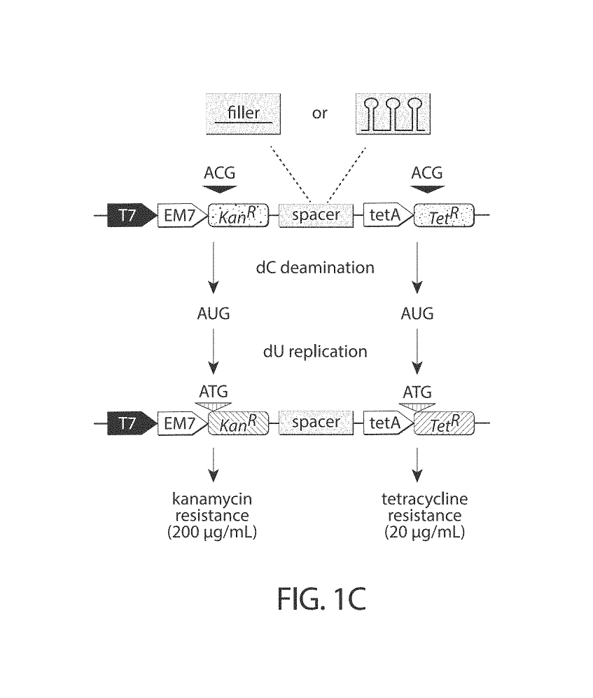

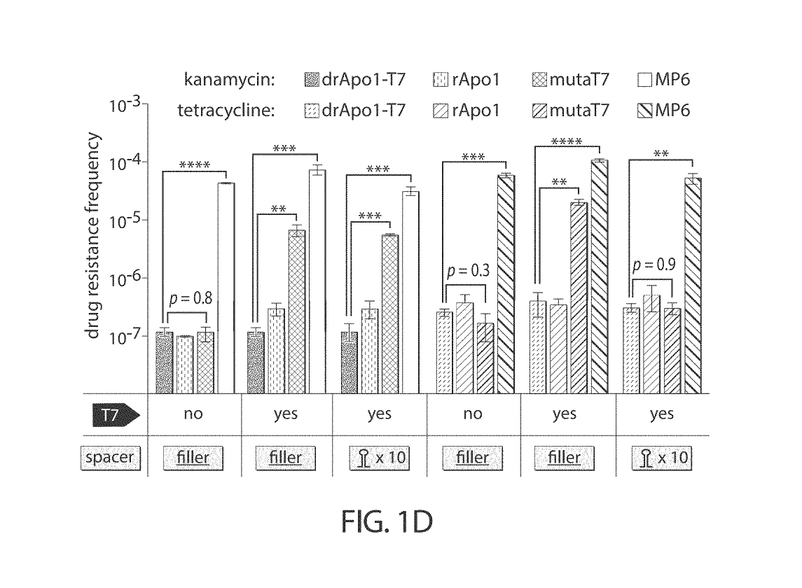

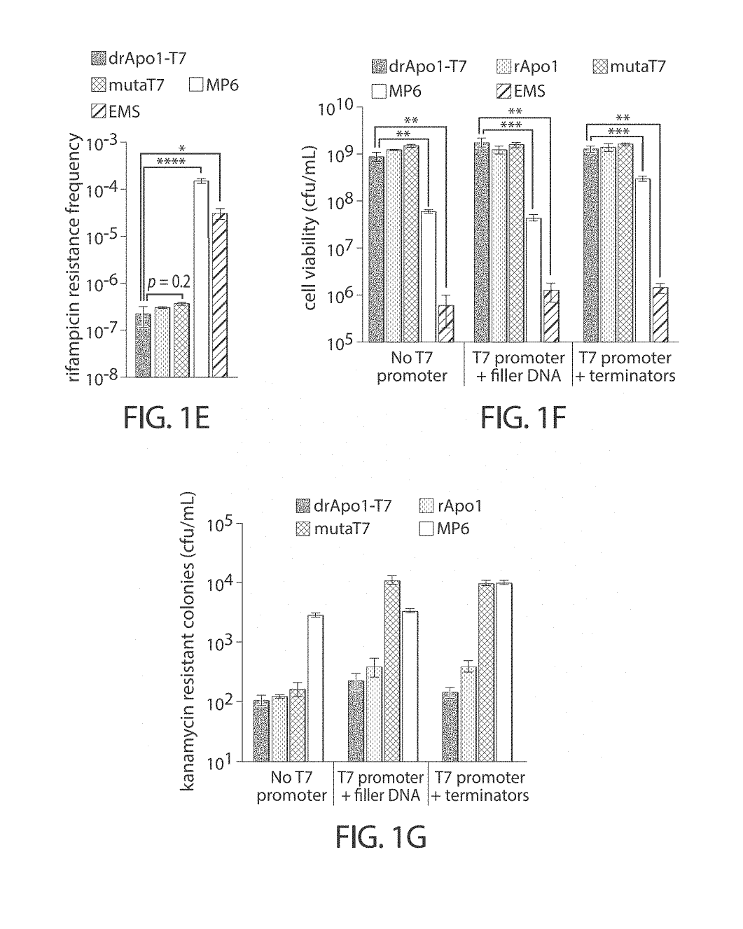

Traditional in vivo mutagenesis strategies, which are especially important for studying and using evolution in living systems, rely on exposing organisms to exogenous mutagens (e.g., high energy light or chemicals (Cupples C. G. and Miller J. H., Proc. Natl. Acad. Sci. U.S.A. 1989 July; 86(14): 5345-49; Tessman et al., Science. 1965 Apr. 23; 148(3669): 507-8)) or expressing mutagenic enzymes in organisms with deficient repair machinery (e.g., XL1-Red (Greener et al., Mol. Biotechnol. 1997 April; 7(2): 189-95) or the MP6 plasmid (Badran A. H. and Liu D. R., Nat. Commun. 2015 Oct. 7; 6: 8425). These global mutagenesis strategies can yield high mutation rates and diverse genetic landscapes. However, the extensive occurrence of mutations throughout the genome is problematic for many experiments, especially directed evolution (FIG. 1A). Off-target mutations outside the intended DNA region are often toxic when they occur in the many essential portions of the genome (Gerdes et al., J. Bacteriol. 2003 October; 185(19): 5673-84; Wang et al., Science. 2015 Nov. 27; 350(6264): 1096-101), a problem that can severely limit library size and even lead to rapid silencing of mutagenic plasmids. Moreover, global mutagens potentiate the emergence of “parasite” variants outside the gene of interest that can circumvent selection schemes (Badran A. H. and Liu D. R., Curr. Opin. Chem. Biol. 2015 February; 24: 1-10). Targeted in vivo mutagenesis strategies have the potential to overcome these deficiencies. For example, DNA-damaging enzymes fused to deactivated Cas9 nucleases can edit bases at specific genetic loci while minimizing off-target mutations (Komor et al., Nature. 2016 May 19; 533(7603): 420-24). Such methods enable targeting of diverse genomic sites but require significant engineering to tile mutagenic enzymes throughout the target DNA (Hess et al., Nat. Methods. 2016 December; 13(12): 1036-42), engineering that must be repeated after each successive round of evolution.

As described herein, Dynamic Targeted Hypermutation (DTH) involves the implementation of a nucleobase-editing enzyme to create genetic diversity in a specific target region of a polynucleic acid sequence. In some embodiments, the methodology facilitates continuous directed evolution in a living system. By mutating specific regions of a polynucleic acid in a targeted fashion, these methodologies reduce off-target mutations that result in cell death or “cheating” of the selection scheme in the directed evolution platform (FIG. 1A). This reduction of off-target mutagenesis results in the production of sequence libraries of unprecedented scale with fewer false positives due to cheating, which translates to an increased probability of discovering an improved product or to the discovery of better final products of the continuous directed evolution process in a shorter amount of time.

In some aspects, the disclosure relates to nucleobase-editing fusion proteins. The nucleobase editing enzymes described herein are capable of altering nucleobases of (or introducing nucleobase mutations in) a pre-existing polynucleic acid sequence (as distinguished from the introduction of mutations during polynucleic acid synthesis, which leaves the parent strand unchanged). In some embodiments, the nucleobase-editing fusion protein can introduce mutations in the 5′ to 3′ direction of a polynucleic acid sequence. In some embodiments, the nucleobase-editing fusion protein can introduce mutations in the 3′ to 5′ direction of a polynucleic acid sequence. In some embodiments, the nucleobase enzyme can introduce mutations in the 5′ to 3′ and the 3′ to 5′ direction of a polynucleic acid sequence. In some embodiments, a nucleobase-editing fusion protein comprises a polynucleic acid-binding protein fused to a nucleobase-editing enzyme.

As used herein, the term “polynucleic acid-binding protein” refers to a protein that binds to specific polynucleic acid sequences. Examples of DNA binding proteins are known to those having skill in the art and include, but are not limited to, polymerases, ligases, reverse transcriptases, nucleases, methyltransferases, glycosylases, helicases, transcription factors, and transcription repressors.

In some embodiments, the polynucleic-acid binding protein is a processive enzyme. The term “processive enzyme” as used herein refers to an enzyme that catalyzes consecutive reactions without releasing its substrate (e.g., in the context of a polymerase, processivity relates to the average number of nucleotides added by the polymerase enzyme per association event with the template strand). Examples of processive enzymes include, but are not limited to, RNA polymerases, DNA polymerases, DNA methyltransferases, DNA glycosylases, and DNA helicases. In some embodiments, the processive enzyme is an RNA polymerase, a DNA polymerase, a DNA methyltransferase, a DNA glycosylase, a DNA helicase, or a functional variant thereof. In some embodiments, the processive enzyme is an RNA polymerase. Examples of RNA polymerases are known to those having skill in the art and include, but are not limited to, T7 RNA polymerase, T3 RNA polymerase, and SP6 RNA polymerase. In some embodiments, the processive enzyme is T7 RNA polymerase or a functional variant thereof.

As used herein, the term “nucleobase-editing enzyme” refers to an enzyme that catalyzes the conversion of a nucleobase to a different nucleobase. Examples of nucleobase-editing enzymes are known to those having skill in the art and include, but are not limited to, Apobec proteins (conversion of cytosine to uracil), TadA proteins (conversion of adenosine to inosine), AMPD proteins (conversion of adenosine to inosine), CDA proteins (conversion of cytidine to uridine), ADAT proteins (conversion of adenosine to inosine), ADAR proteins (conversion of adenosine to inosine), ADA proteins (conversion of adenosine to inosine), and GDA proteins (conversion of guanine to xanthine). In some embodiments, the nucleobase-editing enzyme is selected from the group consisting of an Apobec protein, a TadA protein, an AMPD protein, a CDA protein, an ADAT protein, an ADAR protein, a GDA protein, or a functional variant thereof.

As used herein, the term “Apobec protein” refers to a protein family of deaminases, capable of mutagenizing DNA and/or RNA through the conversion of cytosine to uracil. Apobec proteins have been identified in various species and are known to those having skill in the art. For example, human genes encoding an Apobec protein include APOBEC1, APOBEC2, APOBEC3A, APOBEC3B, APOBEC3C, APOBEC3D (or APOBEC3E), APOBEC3F, APOBEC3G, APOBEC3H, APOBEC4, and Activation-Induced cytidine deaminase. The ability of Apobec proteins to mutagenize DNA and/or RNA varies. For example, some Apobec proteins appear to lack deaminase activity (e.g., APOBEC2). Others are highly mutagenic (e.g., APOBEC3G and rApobec1). The term “Apobec protein” as used herein encompasses all known and currently identifiable Apobec proteins and functional variants thereof. In some embodiments, the Apobec protein is rApobec1 or a functional variant thereof.

As used herein, the term “TadA protein” refers to a family of tRNA-specific adenosine deaminases. TadA proteins have been identified in various species and are known to those having skill in the art. For example, human genes encoding a TadA protein include ADAT1 and ADAT2. E. coli TadA and mouse ADA are additional examples. In some embodiments, the TadA protein is ADAT1, ADAT2, E. coli TadA, ADA, or a functional variant thereof.

As used herein, the term “AMPD protein” refers to a family of adenosine deaminases. AMPD proteins have been identified in various species and are known to those having skill in the art. For example, human genes encoding an AMPD protein include AMPD1, AMPD2 and AMPD3. In some embodiments, the AMPD protein is AMPD1, AMPD2, AMPD3, or a functional variant thereof.

As used herein, the term “CDA protein” refers to a family of cytidine deaminases. CDA proteins have been identified in various species and are known to those having skill in the art. For example, human genes encoding a CDA protein include CDA. In some embodiments, the CDA protein is human CDA or a functional variant thereof.

As used herein, the term “ADAR protein” refers to a family of adenosine deaminases. ADAR proteins have been identified in various species and are known to those having skill in the art. For example, human genes encoding an ADAR protein include ADAR1 and ADAR2. In some embodiments, the ADAR protein is ADAR1, ADAR2 or a functional variant thereof.

As used herein, the term “GDA protein” refers to a family of guanine deaminases. GDA proteins have been identified in various species and are known to those having skill in the art. For example, human genes encoding a GDA protein include GDA. In some embodiments, the GDA protein is human GDA or a functional variant thereof.

The term “functional variant” includes polypeptides which are about 70% identical, at least about 80% identical, at least about 90% identical, at least about 95% identical, at least about 98% identical, at least about 99% identical, at least about 99.5% identical, or at least about 99.9% identical to a protein's native amino acid sequence (i.e., wild-type amino acid sequence) and which retain functionality.

The term “functional variant” also includes polypeptides which are shorter or longer than a protein's native amino acid sequence by about 5 amino acids, by about 10 amino acids, by about 15 amino acids, by about 20 amino acids, by about 30 amino acids, by about 40 amino acids, by about 50 amino acids, by about 75 amino acids, by about 100 amino acids or more and which retain functionality.

In the context of a processive polynucleic-acid binding protein, the term “retain functionality” refers to a functional variant's ability to catalyze consecutive reactions without releasing its substrate at least about 5%, 10%, 20%, 25%, 30%, 40%, 50%, 60%, 70%, 75%, 80%, 90%, 100%, or more than 100% as efficiently as the respective non-variant (i.e., wild-type) processive polynucleic-acid binding protein. Methods of measuring and comparing processivity are known to those skilled in the art.

In the context of a nucleobase-editing enzyme, the term “retain functionality” refers to a functional variant's ability to catalyze the conversion of a nucleobase to a different nucleobase at least about 5%, 10%, 20%, 25%, 30%, 40%, 50%, 60%, 70%, 75%, 80%, 90%, 100%, or more than 100% as efficiently as the respective non-variant (i.e., wild-type) protein. Methods of measuring and comparing nucleobase conversion rates are known to those having skill in the art.

As used herein, the term “fusion protein” refers to the coupling of two or more polypeptides/peptides. In some embodiments, a fusion protein comprises two or more polypeptides/peptides that are covalently coupled in a single polypeptide chain. Covalently connected fusion proteins typically are produced genetically through the in-frame fusing of the nucleotide sequences encoding for each of the said polypeptides/peptides. Expression of the fused coding sequence results in the generation of a single protein without any translational terminator between each of the polypeptides/peptides. In some embodiments, a fusion protein comprises two or more polypeptides/peptides that are coupled through non-covalent association, such as through dimerization domains like FKBP and FRB which dimerize upon the addition of a small-molecule, rapamycin (DeRose et al., Pflugers Arch. 2013 March; 465(3): 409-17). For example, in some embodiments, the polynucleic-acid binding protein is covalently coupled to FKBP and the nucleobase-editing enzyme is covalently coupled to FRB, which could dimerize (non-covalent association) in the presence of rapamycin. Examples of other dimerizing domains or adaptor proteins that facilitate non-covalent association are known to those having skill in the art.

The nucleobase-editing fusion proteins described and encompassed herein comprise a polynucleic acid-binding protein fused to a nucleobase-editing enzyme. In some embodiments, the nucleobase-editing enzyme is C-terminal to the polynucleic acid-binding protein. In other embodiments, the nucleobase-editing enzyme is N-terminal to the polynucleic acid-binding protein.

In some embodiments, the nucleobase-editing fusion protein comprises more than one nucleobase-editing enzyme and/or more than one polynucleic acid-binding protein, which can be arranged in any manner. For example, a nucleobase-editing fusion protein comprising two nucleobase-editing enzymes (“E”) and one polynucleic acid-binding protein (“B”) may be structured from N-terminus to C-terminus as follows: (i) E-B-E; (ii) E-E-B; or (iii) B-E-E.

In some embodiments, one or more proteins or protein domains are positioned between the fused polynucleic acid-binding protein and the nucleobase-editing enzyme. In some embodiments, the polynucleic acid-binding protein is fused to the nucleobase-editing enzyme through a linker. As used herein, the term “linker” refers to a flexible molecule used to connect two molecules of interest together. In some embodiments, the linker is a hydrophilic linker (e.g., PEG linker). In some embodiments, the linker is a peptide linker. In some embodiments, the peptide linker is an XTEN linker (Schellenberger et al., Nat. Biotechnol. 2009 December; 27(12): 1186-90) or a (GGS)ₙ linker.

In some embodiments, the polynucleic acid-binding protein and the nucleobase-editing enzyme are fused via one or more of the following: (i) a cysteine-cysteine disulfide bond; (ii) intein splicing; and (iii) a covalent linkage from an unnatural amino acid (e.g., alkyne-azide “click” reactions, olefin metathesis, or oxime ligation). In some embodiments, the polynucleic acid-binding protein and the nucleobase-editing enzyme are fused through exposure to cross-linking reagents that react with amino acid side chains, such as perfluoro-aromatic stapling, or reagents like NHS esters or isothiocynates or aldehydes.

In some aspects, the disclosure relates to methods of performing dynamic targeted hypermutation. In some embodiments, the method comprises contacting at least one polynucleic acid with at least one non-naturally occurring nucleobase-editing fusion protein as described above, wherein: (a) each of the at least one non-naturally occurring nucleobase-editing fusion proteins comprises a polynucleic acid-binding protein fused to a nucleobase-editing enzyme; (b) each of the at least one polynucleic acid comprises a target region; and (c) the contacting of the at least one polynucleic acid with the at least one non-naturally occurring nucleobase-editing fusion protein generates mutations at a rate exceeding background mutation rates only in the target region of the at least one polynucleic acid of (b), wherein the background mutation rate of the at least one polynucleic acid of (b) is determined in the absence of the non-naturally occurring nucleobase-editing fusion protein.

As used herein, the term “nucleic acid,” as used herein, refers to a compound comprising a nucleobase and an acidic moiety (e.g., a nucleoside, a nucleotide, or a polymer of nucleotides). As used herein, the terms “polynucleic acid” or “polynucleic acid molecule” are used interchangeably and refer to polymeric nucleic acids (e.g., nucleic acid molecules comprising three or more nucleotides that are linked to each other via a phosphodiester linkage).

Polynucleic acid molecules have various forms. In some embodiments, the polynucleic acid molecule is DNA. In some embodiments, the polynucleic acid molecule is double-stranded DNA. For example, in some embodiments, the DNA is genomic DNA. In some embodiments, the DNA is plasmid DNA. In other embodiments, the polynucleic acid molecule is single-stranded DNA. In some embodiments, the polynucleic acid molecule is RNA. In some embodiments, the polynucleic acid molecule is double-stranded RNA. In other embodiments, the polynucleic acid molecule is single-stranded RNA. In some embodiments, the polynucleic acid is a hybrid between DNA and RNA.

The term “target region” as used herein refers to the polynucleic acid sequence that one seeks to mutagenize. In some embodiments, the target region comprises a gene-coding polynucleic acid sequence. In some embodiments, the gene-coding polynucleic acid sequence encodes for an entire gene or sets of entire genes (e.g., a bacterial operon). In other embodiments, the gene-coding polynucleic acid sequence encodes for a portion of a gene (e.g., a polynucleic acid sequence encoding for a protein domain). As used herein the term “portion of a gene” refers to a polynucleic acid sequence comprising at least 10%, at least 20%, at least 30%, at least 40%, at least 50%, at least 60%, at least 70%, at least 80%, or at least 90% of a gene-coding polynucleic acid sequence.

In some embodiments, the target region comprises a non-coding nucleic acid sequence. In some embodiments, the non-coding nucleic acid sequences comprises the sequence of a regulatory element, an intron, a non-coding functional RNA, a repeat sequence, or a telomere. In some embodiments, the regulatory element is selected from the group consisting of an operator, an enhancer, a silencer, a promoter, a terminator, or an insulator. In some embodiments, the target region comprises a gene-coding and non-coding segment of DNA.

The length of a target region may vary. For example, in some embodiments, the target region is greater than 10,000 nucleotides or base pairs in length, such as at least 20,000, at least 25,000, at least 30,000, at least 40,000, at least 50,000, at least 60,000, at least 70,000, at least 80,000, at least 90,000, at least 100,000, or more nucleotides or base pairs in length. In other embodiments, the target region is between 100 and 10,000 nucleotides or base pairs in length, such 100-200, 200-500, 500-1000, or 1,000-5,000 nucleotides or base pairs in length. In other embodiments, the polynucleic acid molecule region of interest is less than 100 nucleotides or base pairs in length.

In some embodiments, a nucleobase-editing fusion protein generates mutations at a rate exceeding background mutation rates only in the target region (i.e., in polynucleic acid regions outside of the target region, the conversion of cytosine bases to uracil bases remain at background levels). In other embodiments, mutation rates outside of the target region (i.e., background mutation rates) are increased less than 100 percent, less than 90 percent, less than 80 percent, less than 70 percent, less than 60 percent, less than 50 percent, less than 40 percent, less than 30 percent, less than 20 percent, or less than 10 percent in the presence of the nucleobase-editing fusion protein relative to the rate in the absence of the nucleobase-editing fusion protein. Processes contributing to background mutation rates include the spontaneous deamination of cytosine to uracil through hydrolysis and errors in replication or transcription. Methods of measuring mutation rates are known to those having skill in the art.

In some embodiments, the at least one polynucleic acid comprises, from 5′ to 3′: a promoter region that is bound by at least one of the at least one non-naturally occurring nucleobase-editing fusion proteins in a sequence-specific manner; the target region; and a terminator region comprising a terminator array.

In some embodiments, the promoter region of at least one of the at least one polynucleic acids comprises the sequence of SEQ ID NO: 21, SEQ ID NO: 22, and/or SEQ ID NO: 23.

In some embodiments, the terminator array comprises four or more terminators, such as at least four, at least five, at least six, at least seven, at least eight, at least nine, or at least ten terminators. In some embodiments, Rho-independent terminators are used, which can be one or more types of naturally occurring terminators, such as T7 and rrnB, or one or more types of engineered high-efficiency terminators, such as T0. In some embodiments, when using a nucleobase-editing fusion protein containing T7 RNA polymerase, the terminator array comprises at least four, at least five, at least six, at least seven, at least eight, at least nine, or at least ten T7 UUCG terminators.

In some embodiments, the contacting of the at least one polynucleic acid with the at least one non-naturally occurring nucleobase-editing fusion protein occurs in a living cell. In some embodiments, the living cell is a cell of a multicellular organism. In some embodiments the living cell is a unicellular organism. In some embodiments, the unicellular organism is a bacteria. In some embodiments, the bacteria is E. coli.

In some embodiments, the nucleobase-editing fusion protein is encoded for on a plasmid contained within a living cell, wherein the plasmid has copy number of less than 10. In some embodiments the copy number is less than 9, less than 8, less than 7, less than 6, less than 5, less than 4, less than 3, or less than 2.

In some embodiments, the living cell contains a modified genome comprising an integration of a polynucleic acid sequence encoding for and driving the expression of the non-naturally occurring nucleobase-editing fusion protein. In some embodiments, the expression of the non-naturally occurring nucleobase-editing fusion protein is driven by a promoter comprising the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, and/or SEQ ID NO: 24.

In some embodiments, the living cell contains a modified genome comprising an integration of a polynucleic sequence comprising, from 5′ to 3′: a promoter region that is bound by at least one of the at least one non-naturally occurring nucleobase-editing fusion proteins in a sequence-specific manner; the target region; and a terminator region comprising a terminator array.

In some embodiments, the living cell contains a modified genome comprising: an integration of a polynucleic acid sequence encoding for and driving the expression of the non-naturally occurring nucleobase-editing fusion protein; and an integration of a polynucleic sequence comprising, from 5′ to 3′: a promoter region that is bound by at least one of the at least one non-naturally occurring nucleobase-editing fusion proteins in a sequence-specific manner; the target region; and a terminator region comprising a terminator array.

In some embodiments, the expression of at least one of the at least one non-naturally occurring nucleobase-editing fusion proteins can be conditionally controlled. Examples of inducible expression systems that facilitate conditional gene expression are known to those having skill in the art. For example, some inducible expression systems comprise promoters that are chemically regulated (e.g., alcohol-regulated, tetracycline-regulated, steroid-regulated, or metal-regulated. Other inducible expression systems comprise promoters that are physically regulated (e.g., temperature-regulated or light-regulated).

In some embodiments, the living cell contains a modified genome and a plasmid that facilitates expression of a T7 inhibitor, wherein the modified genome of the living cell comprises: (a) an integration of a polynucleic acid sequence encoding for and driving the expression of the non-naturally occurring nucleobase-editing fusion protein, wherein the sequence driving the expression of the fusion protein comprises a sequence bound by LacI repressor that inhibits transcription of the fusion protein when LacI is bound; and (b) a deletion of genomic sequence encoding for uracil deglycosylase. In some embodiments, the T7 inhibitor is T7 lysozyme. As used herein, the term “inhibits transcription” refers to a decrease in the expression of the non-naturally occurring nucleobase-editing fusion protein by about 5%, 10%, 20%, 25%, 30%, 40%, 50%, 60%, 70%, 75%, 80%, 90%, or more than 95% relative to the level of expression in the absence of LacI. Methods of measuring and comparing expression levels are known to those skilled in the art.

In some embodiments, the living cell is treated to increase the expression and/or activity of the uracil deglycosylase inhibitor, ugi (Savva R. and Pearl L. H., Nat. Struct. Biol. 1995 September; 2(9): 752-57). For example, in some embodiments, a plasmid encoding for an expressible uracil deglycosylase inhibitor is delivered to the living cell, and the expression of the uracil deglycosylase inhibitor is stimulated.

In some aspects, the invention relates to kits for performing targeted dynamic hypermutation. In some embodiments, the kit comprises: (a) a polypeptide comprising the amino acid sequence of a non-naturally occurring nucleobase-editing fusion protein comprising a processive polynucleic acid-binding protein fused to a nucleobase-editing enzyme; and (b) a polynucleic acid sequence comprising, from 5′ to 3′: a promoter region that is bound by the non-naturally occurring nucleobase-editing fusion protein of (a) in a sequence-specific manner; a cloning site; and a terminator region comprising a terminator array.

In other embodiments, the kit comprises: (a) a polynucleic acid sequence encoding for and driving the expression of a non-naturally occurring nucleobase-editing fusion protein comprising a processive polynucleic acid-binding protein fused to a nucleobase-editing enzyme; and (b) a polynucleic acid sequence comprising, from 5′ to 3′: a promoter region that is bound by the non-naturally occurring nucleobase-editing fusion protein of (a) in a sequence-specific manner; a cloning site; and a terminator region comprising a terminator array.

In some embodiments, at least one component in the kit is provided in a desiccated or lyophilized form. In other embodiments, at least one component of the kit is provided in a solubilized form. In some embodiments, the kit further comprises at least one buffer. In some embodiments at least one of the at least one buffers is a reaction buffer.

The term “cloning site,” as used herein refers to a segment of DNA that facilitates the cloning of a polynucleic acid comprising a target region. In some embodiments, the cloning site is a multiple cloning site comprising endonuclease restriction sites for restriction-mediated cloning. In some embodiments the cloning site is a TA cloning site. In some embodiments, the cloning site comprises a nucleic acid sequence that facilitates homologous recombination.

In some embodiments, the kit also comprises competent cells for use in the cloning of the target region. For example, in some embodiments, the competent cells are chosen from the list consisting of TOP10, OmniMax, PIR1, PIR2, INV α F, INV110, BL21, Mach1, DH10Bac, DH10B, DH12S, DH5α, Stb12, Stb13, and Stb14. XL1-Blue, XL2-Blue, and related strains.

### EXAMPLES

### Example 1

### Implementation of Dynamic Targeted Hypermutation

The implementation of dynamic target hypermutation (DTH) depends on the action of a polynucleic acid-binding protein fused to a nucleobase-editing enzyme, such as an RNA polymerase combined with a cytidine deaminase. To demonstrate the DTH methodology, RNA polymerase from a bacteriophage (T7) was fused to cytidine deaminase from Rattus norvegicus (rApobec1) to form various rApo1−T7 constructs. These constructs specifically bind to a sequence of DNA called the T7 promoter, which is positioned adjacent to the target sequence of DNA (TABLE 1). Various constructs were engineered and tested in multiple reporter assays (TABLE 1 and TABLE 2).

When rApo1−T7 initiates transcription at the promoter site, the DNA of the target sequence is exposed and altered by the action of the T7 RNA polymerase and altered by the rApo1 domain. Since the T7 polymerase of rApo1−T7 is processive, it continues to travel along the DNA target sequence until it reaches a terminating sequence at the end of the DNA target sequence. Importantly, data disclosed herein demonstrate that rApo1−T7 has a high mutation rate and low toxicity relative to global methods (mutagenic plasmid [MP6], which is the current gold standard for in vivo global mutagenesis methods).

Additional components can provide further constraints so that mutations are limited to a defined stretch of DNA (see Examples 2-4). These constraints (and their underlying importance to the implementation of DTH) have not been demonstrated previously.

TABLE 1
List of constructs tested with accompanying expectations and observations.

| | | | | DNA Sequences | | | |
|---|---|---|---|---|---|---|---|
| | | | | Copy | | | |
| | Summary | Plasmid Name | Origin | Number | Promoter | Construct | Linker |
| 1 | Highly toxic and | pTara | p15a | ~10 | pBAD | rApo1-X- | 16 residue |
| | inconclusive | | | | (arabinose | T7 | XTEN linker |
| | results | | | | inducible) | rApo1-G1- | (GGS)₂ |
| | | | | | | T7 | (SEQ ID NO: 99) |
| | | | | | | rApo1-G2- | (GGS)₄ |
| | | | | | | T7 | (SEQ ID NO: 100) |
| 2 | Poor activity | pTSara (Split | p15a | ~10 | pBAD | rApo1-X- | 16 residue |
| | | T7, one | | | (arabinose | T7-c | XTEN linker |
| | | plasmid) | | | inducible) | rApo1-G1- | (GGS)₂ |
| | | | | | | T7 | (SEQ ID NO: 99) |
| | | | | | | rApo1-G2- | (GGS)₄ |
| | | | | | | T7 | (SEQ ID NO: 100) |
| 2.5 | Poor activity | pTSara (Split | p15a | ~10 | pBAD | rApo1-X- | 16 residue |
| | | T7, c-Term | | | (arabinose | T7 | XTEN linker |
| | | only fused to | | | inducible) | rApo1-G1- | (GGS)₂ |
| | | rApo1) | | | | T7 | (SEQ ID NO: 99) |
| | | | | | | rApo1-G2- | (GGS)₄ |
| | | | | | | T7 | (SEQ ID NO: 100) |
| | | puc (Split T7, | puc | ~700 | pTac | T7-n | |
| | | n-Term only) | | | (Strong | fragment | |
| | | | | | promoter) | | |
| 3 | Highly toxic, | pTara | p15a | ~10 | pBAD | rApo1-X- | 16 residue |
| | mutagenesis prior | | | | (arabinose | T7 | XTEN linker |
| | to induction (low | | | | inducible) | rApo1-G1- | (GGS)₂ |
| | expression) | | | | | T7 | (SEQ ID NO: 99) |
| | | | | | | rApo1-G2- | (GGS)₄ |
| | | | | | | T7 | (SEQ ID NO: 100) |
| | | | | | | rApo1- | (GGGGS)₁₃ |
| | | | | | | 13X-T7 | (SEQ ID NO: 101) |
| 4 | T7 activity is | pTara | p15a | ~1-2 | Anderson | rApo1-G1- | (GGS)₂ |
| | slightly inducible, | | | | (Weak IPTG | T7 | (SEQ ID NO: 99) |
| | unclear if | | | | inducible | rApo1-G2- | (GGS)₄ |
| | mutagenesis is | | | | promoters) | T7 | (SEQ ID NO: 100) |
| | inducible. | | | | (TABLE 3) | | |
| 5 | Mutagenesis | Genomic | n/A | ~1 | Anderson | rApo1-G1- | (GGS)₂ |
| | observed, but it | integration of | | | (Weak IPTG | T7 | (SEQ ID NO: 99) |
| | was not inducible, | constructs | | | inducible | rApo1-G2- | (GGS)₄ |
| | No toxicity. | | | | promoters) | T7 | (SEQ ID NO: 100) |
| | | | | | (TABLE 3) | | |
| 6 | Significant | Genomic | n/a | 1 | P_{A1lacO-Tenth}, | rApo1-G1- | (GGS)2 |
| | mutagenesis with | integration of | | | (Weak | T7 | (SEQ ID NO: 99) |
| | or without IPTG, | constructs with | | | IPTG | rApo1-G2- | (GGS)₄ |
| | A large number of | a weaker | | | inducible | T7 | (SEQ ID NO: 100) |
| | viable mutants | promoter, | | | promoter) | | |
| | were obtained per | deletion of | | | (SEQ ID | | |
| | mL relative to | UNG (uracil | | | NO: 24) | | |
| | MP6. | deglycosylase) | | | | | |
| | | from genome, | | | | | |
| | | insertion of | | | | | |
| | | strong promoter | | | | | |
| | | for LacI | | | | | |
| | | repressor | | | | | |
| 7 | Tunable | Genomic | n/a | 1 | P_{A1lacO-Tenth}, | rApo1-G1- | (GGS)₂ |
| | mutagenesis, but | integration of | | | (Weak IPTG | T7 | (SEQ ID NO: 99) |
| | not completely | constructs, | | | inducible | rApo1-G2- | (GGS)₄ |
| | repressible | deletion of | | | promoter) | T7 | (SEQ ID NO: 100) |
| | | UNG (uracil | | | (SEQ ID | | |
| | | deglycosylase) | | | NO: 24) | | |
| | | from genome, | | | | | |
| | | insertion of | | | | | |
| | | strong promoter | | | | | |
| | | for LacI | | | | | |
| | | repressor. | | | | | |
| | | Introduction of | | | | | |
| | | a plasmid that | | | | | |
| | | expresses a T7 | | | | | |
| | | inhibitor (i.e. | | | | | |
| | | T7 lysozyme) | | | | | |

| | Summary | Analyses | Expectation | Observations |
|---|---|---|---|---|
| 1 | Highly toxic and | Lac | Arabinose | The appearance of blue |
| | inconclusive | reporter | induces | colonies indicates that T7 |
| | results | disruption | expression | is still active when fused |
| | | assay and | of | to rApo1. The constructs |
| | | T7 | mutagenic | were expressed and quite |
| | | activity | constructs | toxic prior to arabinose |
| | | assay | resulting in | treatment, resulting in |
| | | | the | reduced number of |
| | | | appearance | colonies. Subsequent |
| | | | of white | treatment with arabinose |
| | | | colonies. | increased expression and |
| | | | | toxicity of rApo1 |
| | | | | constructs, resulting in no |
| | | | | colonies. XTEN was an |
| | | | | exception to toxicity |
| | | | | trend, though no T7 |
| | | | | activity was observed. |
| 2 | Poor activity | Lac | Arabinose | Split T7 activity appeared |
| | | reporter | induces | to be abolished when |
| | | disruption | expression | rApo1 was fused to T7-c |
| | | assay and | of | fragment. |
| | | T7 | mutagenic | |
| | | activity | constructs | |
| | | assay | resulting in | |
| | | | the | |
| | | | appearance | |
| | | | of white | |
| | | | colonies. | |
| 2.5 | Poor activity | T7 | N-terminus | Overexpression of N-term |
| | | activity | fragment is | fragment of T7 restored |
| | | assay | always | minimal amount of T7 |
| | | | high. | activity. |
| | | | Arabinose | |
| | | | induces | |
| | | | expression | |
| | | | of C- | |
| | | | terminus | |
| | | | mutagenic | |
| | | | constructs, | |
| | | | resulting in | |
| | | | the | |
| | | | appearance | |
| | | | of blue | |
| | | | colonies. | |
| 3 | Highly toxic, | No-Start | Arabinose | Arabinose induction |
| | mutagenesis prior | Kan | induces | killed everything except |
| | to induction (low | Resistance | expression | XTEN. Water and glucose |
| | expression) | | of | (weak expression and |
| | | | mutagenic | repression of expression, |
| | | | constructs, | respectively) resulted in |
| | | | resulting in | mild mutagenesis for G1 |
| | | | the | and G2. Very little/no |
| | | | appearance | mutagenesis for 13X and |
| | | | of more | XTEN when compared to |
| | | | colonies on | background. |
| | | | kanamycin | |
| | | | treated | |
| | | | plates. | |
| 4 | T7 activity is | T7 | Only | Prior to induction, the |
| | slightly inducible, | activity | observe T7 | tenth strength (SEQ ID |
| | unclear if | assay | activity | NO: 16) promoter |
| | mutagenesis is | | with IPTG | colonies were already |
| | inducible. | | induction | blue, and hundredth |
| | | | and | strength (SEQ ID NO: 15) |
| | | | minimal | was slightly blue. |
| | | | toxicity. | Induction with IPTG |
| | | | | resulted in death of |
| | | | | bacteria with tenth |
| | | | | strength promoter. IPTG+ |
| | | | | colonies grew for |
| | | | | hundredth strength |
| | | | | constructs and were quite |
| | | | | blue. |
| 5 | Mutagenesis | No-Start | Arabinose | Observed mutagenesis 4 |
| | observed, but it | Kan | induces | to 5-fold above |
| | was not inducible, | Resistance | expression | background mutations |
| | No toxicity. | | of | with and without IPTG. |
| | | | mutagenic | There was no observed |
| | | | constructs, | toxicity. |
| | | | resulting in | |
| | | | the | |
| | | | appearance | |
| | | | of more | |
| | | | colonies on | |
| | | | kan-treated | |
| | | | plates. | |
| 6 | Significant | No-Start | IPTG | Approximately 1000-fold |
| | mutagenesis with | Kan | induces | increase in number of |
| | or without IPTG, | Resistance | expression | Kan-resistant colonies |
| | A large number of | | of | observed with IPTG, |
| | viable mutants | | mutagenic | about 100-fold without |
| | were obtained per | | constructs | IPTG, with minimal |
| | mL relative to | | resulting in | toxicity compared to a |
| | MP6. | | the | global mutagen (MP6). |
| | | | appearance | There were 10X more |
| | | | of more | Kan-resistant colonies per |
| | | | colonies on | unit volume with rApoI- |
| | | | kan-treated | GGS-T7 than with MP6. |
| | | | plates. | |
| 7 | Tunable | No-Start | IPTG | Observed approximately a |
| | mutagenesis, but | Kan | induces | 5-fold difference between |
| | not completely | Resistance | expression | mutagenesis without |
| | repressible | | of | IPTG and with IPTG. |
| | | | mutagenic | Without IPTG, |
| | | | constructs | mutagenesis is still above |
| | | | resulting in | background. |
| | | | the | |
| | | | appearance | |
| | | | of more | |
| | | | colonies on | |
| | | | kan-treated | |
| | | | plates. | |

TABLE 2
List of assays used to test rApo1-T7 constructs.

| Assay Name | Purpose | Description of Assay |
|---|---|---|
| T7 activity assay | Measurement of the | LacZ alpha fragment acts as a reporter that turns colonies |
| | activity of the T7 | blue in the presence of X-gal. In this assay, expression of |
| | RNA polymerase | the LacZ alpha fragment is driven solely by a T7 promoter. |
| | | The colonies will only turn blue if the variant of the T7 |
| | | RNA polymerase is active. If the variant is inactive, the |
| | | colonies will stay white. |
| Lac reporter disruption | Measure mutation | LacZ alpha fragment acts as a reporter that turns colonies |
| assay | frequency (Screen) | blue in the presence of X-gal. As mutations accumulate in |
| | | the LacZ gene it is disrupted and no longer functions, |
| | | resulting in the appearance of white colonies. |
| No-Start Kanamycin | Measure mutation | The kanamycin phosphotransferase gene lacks a start |
| Resistance | frequency | codon, so it is initially silent. Only bacterial cells that |
| | (Selection) | mutate the silent codon back into a start codon gain |
| | | resistance to kanamycin. |
| No-Start | Measure on-/off- | Two genes without start codons are present in a plasmid. |
| Kanamycin/Tetracycline | target mutation | The first encodes kanamycin phosphotransferase gene, |
| Resistance | frequencies | which provides resistance to kanamycin when the silent |
| | (Selection) | codon is reverted to a start codon. A second gene encodes |
| | | a tetracycline exporter gene, which provides resistance to |
| | | tetracycline when it's silent codon is changed to a start |
| | | codon. By targeting mutagenesis to one gene and |
| | | measuring the rate at which resistant colonies appear in |
| | | populations, the mutation frequencies at these two sites can |
| | | be used to calculate the relative ratio of on-target and off- |
| | | target mutagenesis. |

### Example 2

### Decreasing Mutagenesis Downstream of the Target DNA

Targeted mutagenesis is defined as the constraint of mutations to a defined stretch of DNA. In other words, mutations should not appear outside of the target region. In the implementation of rApo1−T7 demonstrated in Example 1, one might expect that the mutation frequency upstream of the T7 promoter would be very low. However, preventing mutations downstream of the target region could be a tremendous challenge. Previous data has shown that monomeric RNA polymerases can be quite processive and carry out transcription for exceptionally long stretches of DNA—in excess of 20 kb in the case of T7 RNA polymerase (Rong et al., J. Biol. Chem. 1998 Apr. 24; 273(17): 10253-60; Thiel et al., J. Gen. Virol. 2001 June; 82(Pt 6): 1273-81). Effective termination of transcription is further complicated by the context-dependent nature of termination efficiency (Mairhofer et al., ACS Synth. Biol. 2015 Mar. 20; 4(3): 265-73).

An unsuccessful termination event for rApo1−T7 can result in the incorporation of undesired mutations throughout many kilobases of DNA downstream of the target region. These undesired mutations are typically catastrophic in the context of directed evolution, as these changes can produce numerous variants outside of the gene of interest that overcome a selection scheme in a living organism (i.e., “cheaters”). Previous attempts at technologies similar to DTH have failed to address or entirely ignored undesired mutagenesis downstream of the target region or elsewhere in the genome. Therefore, experiments were designed to test the possibility of off-target mutagenesis, and if necessary eliminate it.

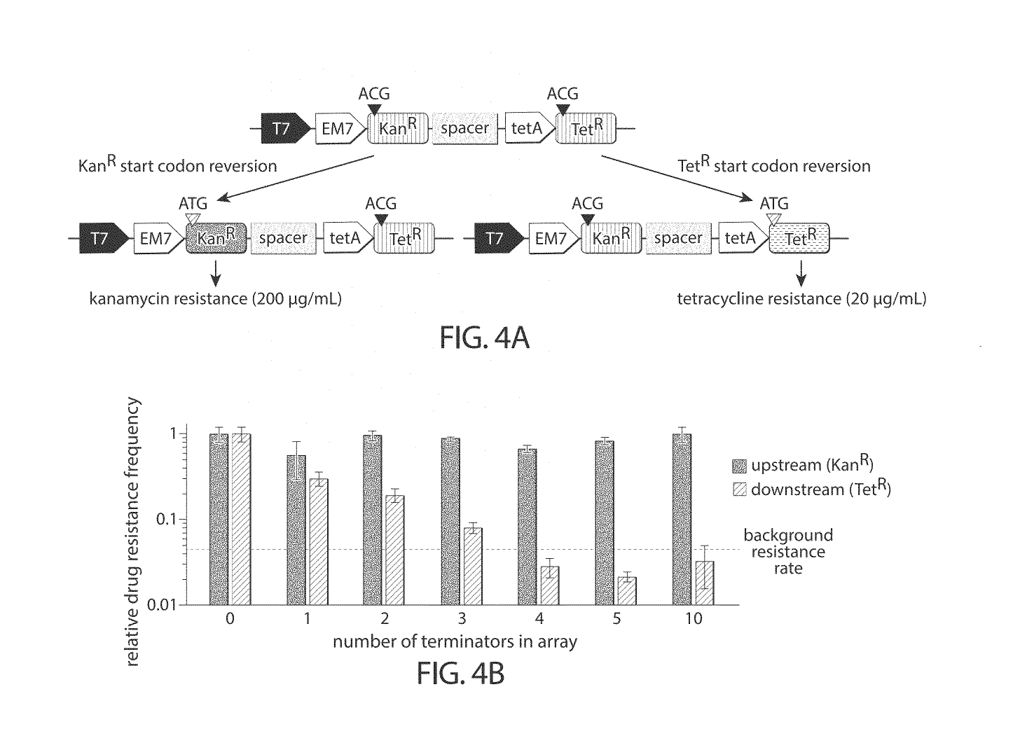

A start codon reversion drug resistance assay was designed in which two drug resistance genes were positioned in series, both of which lacked a start codon (FIG. 4A). When only filler DNA (no T7 UUCG terminator) separated the two drug resistance genes, there was minimal termination of rApo1−T7. Indeed, similar frequencies of start codon reversion were observed for both drug resistance genes (FIG. 4B). Inserting a single terminator between the drug resistance genes resulted in only a minor reduction in the frequency of start codon reversion in the downstream gene compared to upstream gene (FIG. 4B). Likewise, inclusion of 2 or 3 common terminators decreased, but did not prevent off-target mutagenesis carried out by rApo1−T7 (FIG. 4B). Indeed, use of 1 to 3 common terminators common for T7 in DTH results in mutagenesis for a long distance downstream of the target DNA. Subsequent engineering of larger terminator arrays (up to 10 copies) further reduced the mutagenesis frequency in downstream genes to background levels while still accumulating mutations in gene upstream of the terminator array (FIG. 4B).

Additional experiments were performed using LacO operons recruiting the Lac repressor to interfere with T7 processivity and promote termination; however, these limited attempts were unsuccessful.

### Example 3

### Minimal Expression of rApo1−T7

Similar to termination, it was found that expression levels of rApo1−T7 can result in untargeted mutagenesis if left unchecked. In preliminary implementations of the DTH using rApo1−T7, significant cytotoxicity was observed even when rApo1−T7 was expressed under limiting conditions through a common promoter (such as an arabinose inducible promoter with glucose suppression). In the context of directed evolution, such widespread changes results in the regular appearance of “cheaters.”

Thus, experiments were designed to limit the expression of rApo1−T7 by alternative strategies beyond traditional promoters. In the most successful implementation, the combined effects of reducing promoter strength (TABLE 3; Registry of Standard Biological Parts, parts.igem.org/Promoters/Catalog/Anderson; Camsund et al., J. Biol. Eng. 2014 Jan. 27; 8(1): 4) and limiting copy number of the rApo−T7 gene were critical for limiting cytotoxicity when utilizing rApo1−T7 in E. coli. Expression of rApo1−T7 constructs under medium copy number conditions was highly toxic. Moreover, use of split T7 to increase mutagenesis and reduce toxicity failed because T7 polymerase activity of the split constructs was unacceptably low.

TABLE 3
List of potential promoter sequences for driving the expression
of a nucleobase-editing fusion protein and their accompanying strengths.

| | | SEQ ID | Measured |
|---|---|---|---|
| Identifier | Sequence | NO | Strength |
| BBa_J23119 | ttgacagctagctcagtcctaggtataatgctagc | 1 | n/a |
| BBa_J23100 | ttgacggctagctcagtcctaggtacagtgctagc | 2 | 1 |
| BBa_J23101 | tttacagctagctcagtcctaggtattatgctagc | 3 | 0.70 |
| BBa_J23102 | ttgacagctagctcagtcctaggtactgtgctagc | 4 | 0.86 |
| BBa_J23103 | ctgatagctagctcagtcctagggattatgctagc | 5 | 0.01 |
| BBa_J23104 | ttgacagctagctcagtcctaggtattgtgctagc | 6 | 0.72 |
| BBa_J23105 | tttacggctagctcagtcctaggtactatgctagc | 7 | 0.24 |
| BBa_J23106 | tttacggctagctcagtcctaggtatagtgctagc | 8 | 0.47 |
| BBa_J23107 | tttacggctagctcagccctaggtattatgctagc | 9 | 0.36 |
| BBa_J23108 | ctgacagctagctcagtcctaggtataatgctagc | 10 | 0.51 |
| BBa_J23109 | tttacagctagctcagtcctagggactgtgctagc | 11 | 0.04 |
| BBa_J23110 | tttacggctagctcagtcctaggtacaatgctagc | 12 | 0.33 |
| BBa_J23111 | ttgacggctagctcagtcctaggtatagtgctagc | 13 | 0.58 |
| BBa_J23112 | ctgatagctagctcagtcctagggattatgctagc | 14 | 0.00 |
| BBa_J23113 | ctgatggctagctcagtcctagggattatgctagc | 15 | 0.01 |
| BBa_J23114 | tttatggctagctcagtcctaggtacaatgctagc | 16 | 0.10 |
| BBa_J23115 | tttatagctagctcagcccttggtacaatgctagc | 17 | 0.15 |
| BBa_J23116 | ttgacagctagctcagtcctagggactatgctagc | 18 | 0.16 |
| BBa_J23117 | ttgacagctagctcagtcctagggattgtgctagc | 19 | 0.06 |
| BBa_J23118 | ttgacggctagctcagtcctaggtattgtgctagc | 20 | 0.56 |

The unbolded nucleotides are part of the consensus promoter sequence (BBa_J23119) among all promoters while the bold nucleotides highlight the differences between the individual promoters and the consensus sequence.

TABLE 4
List of potential promoter sequences that can be
bound by nucleobase-editing fusion proteins.

| | | SEQ | |
|---|---|---|---|
| Identifier | Sequence | ID NO | Bound By |
| T7 | TAATACGACTCACTATAGG | 21 | T7 RNA |
| Promoter | | | polymerase |
| T3 | AATTAACCCTCACTAAAGG | 22 | T3 RNA |
| Promoter | | | polymerase |
| SP6 | ATTTAGGTGACACTATAGA | 23 | SP6 RNA |
| Promoter | | | polymerase |

### Example 4

### Inducible Expression of rApo1−T7

No previously described mutagenesis methodology has demonstrated conditional control that allows users to conveniently turn on and shut off targeted mutation accumulation in a living organism. While MP6 inducible system have been disclosed (Badran A. H. and Liu D. R., Nat. Commun. 2015 Oct. 7; 6: 8425), it carries out mutagenesis globally. Commonly used non-inducible mutagenesis methods used in living organisms are designed to continuously carry out global mutagenesis, which forces users to isolate the final libraries of evolved genes of interest from mutagenic organisms and subsequently transfer these libraries to a non-mutagenic organism for downstream sequencing and characterization. Conditional control of mutagenesis would allow users to switch off targeted mutagenesis after a desired portion of time, effectively eliminating the need to isolate and transfer evolved libraries from one organism to another.

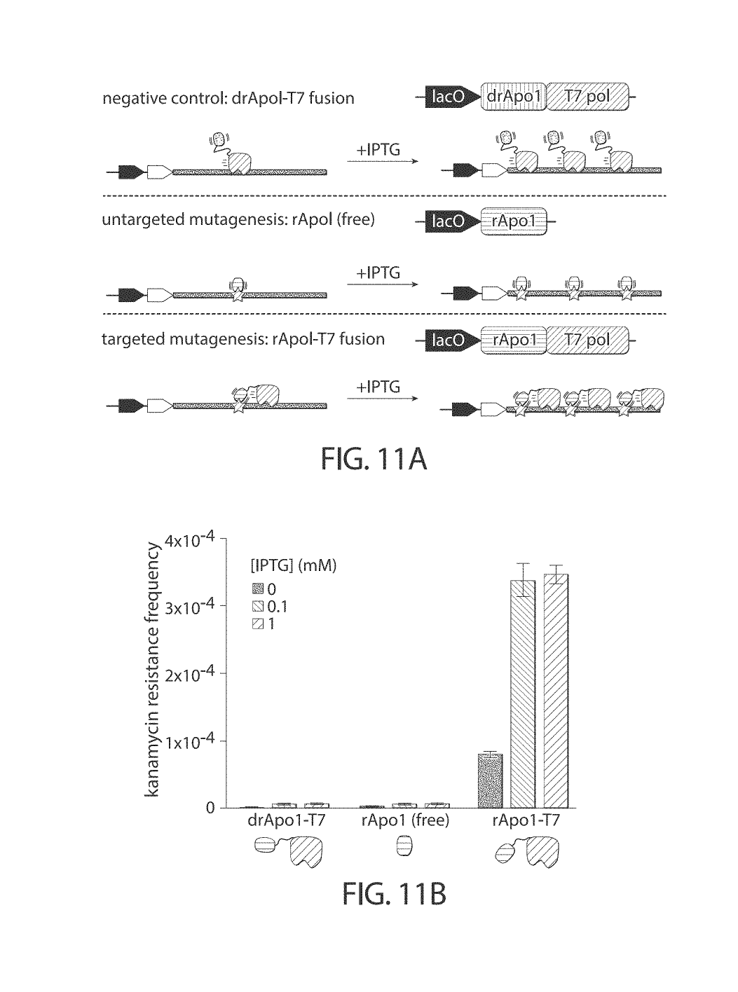

The results disclosed herein demonstrate that the activity of rApo−T7 can be conditionally tuned by chemically inducing the expression of LacI-repressed rApo1−T7 with IPTG, such that higher expression levels of T7 polymerase correlate with increased levels of mutagenesis (FIGS. 11A-11B). Optimization of conditional promoter strength and copy number was required to avoid uninduced mutagenesis and cellular toxicity. For example, expression under full-strength conditional promoters showed leaky expression when uninduced and toxicity when induced. Likewise, reducing promoter strength failed to address leaky expression and lack of induction when expressing from a medium copy plasmid. By further tuning the expression of T7 lysozyme—a T7 inhibitor—relative to T7 RNA polymerase and optimizing the interaction between both partners, it is likely that mutagenesis levels under repressive conditions will fall to background levels.

### Example 5

### Materials and Methods for Examples 6 and 7

General Methods: All PCR reactions for restriction cloning and recombineering targeting cassettes were performed using Q5 High Fidelity DNA Polymerase (New England Biolabs). Primers were ordered from Life Technologies and g-blocks were ordered from Integrated DNA Technologies.

Chemicals: Kanamycin monosulfate was purchased as a solid from Alfa Aesar (J61272). Tetracycline hydrochloride was purchased as a solid from Calbiochem (58346). Fosfomycin was purchased as a solid from Alfa Aesar (J6602). Rifampicin was purchased as a solid from TCI (R0079). Ampicillin was purchased as a solid sodium salt form Fisher bioreagents (BP1761-25). Streptomycin sulfate was purchased as a solid from MP Biomedicals (100556). Chloramphenicol was purchased as a solid from Alfa Aesar (B20841). Tetrazolium chloride was purchased as a solid from Aldrich (T8877). L-rhamnose was purchased as a solid from Sigma-Aldrich (W373011). L-arabinose was purchased as a solid form Chem Impex (01654). Isopropyl β-D-1-thiogalactopyranoside (IPTG) was purchased as a solid from Sigma-Aldrich (16758-1G). Antifoam 204 was purchased as liquid from Sigma (A8311-50ML). LB was purchased as a solid form Difco (244620). Agar was purchased as a solid from Alfa Aesar (A10752). Cycloheximide was purchased as a solid from Chem Impex (00083). Ethylmethanesulfonate (EMS) was purchased from Sigma Aldrich (M0880-1G).

Cloning: All plasmids were generated by restriction cloning. Ligation reactions were performed using Quick Ligase (New England Biolabs). All DNA cloning was performed in DH10B cells (Invitrogen). The rApo1 gene was amplified from pET28b-BE1 (Komor et al., Nature. 2016 May 19; 533(7603): 420-24) and the T7 RNA polymerase gene was amplified from pTara (Wycuff D. R. and Matthews K. S., Anal. Biochem. 2000 Jan. 1; 277(1): 67-73). Mutation assay reporter plasmids utilize the single-copy BAC origin and the terminator arrays of the UUCG-T7 derivative of the T7 terminator (Mairhofer et al., ACS Synth. Biol. 2015 Mar. 20; 4(3): 265-73), were generated by serial insertion of the annealed oligos NheI-UUCG-BamHI S and NheI-UUCG-BamHI AS.

TABLE 5
Strain table. The genotypes of strains used in this work
are shown. The “x<>y” notation indicates
a replacement of “x” with “y” through lambda red recombineering.

| Strain | Genotype |
|---|---|
| DH10B | F⁻ araD139 Δ(araA-leu)7697 ΔlacX74 galE15 galK16 galU |
| (Durfee et | hsdR2 relA1 rpsL150(Str^{R}) spoT1 φ80lacZΔM15 endA1 |
| al., J. | nupG recA1 e14⁻ mcrA Δ(mrr hsdRMS-mcrBC) |
| Bacteriol. | |
| 2008 April; | |
| 190(7): | |
| 2597-606) | |
| Δung | DH10B Δung ΔmotAB ΔcsgABCDEFG |
| rApo1 | DH10B Δung ΔmotAB ΔcsgABCDEFG [P_{lacI}<>P_{tac}] |
| | Δ(araA-leu)7697::[P_{A1lacO-Tenth} rApo1] |
| drApo1 | DH10B Δung ΔmotAB ΔcsgABCDEFG [P_{lacI}<>P_{tac}] |
| | Δ(araA-leu)7697::[P_{A1lacO-Tenth} drApo1] |
| MutaT7 | DH10B Δung ΔmotAB ΔcsgABCDEFG [P_{lacI}<>P_{tac}] |
| | Δ(araA-leu)7697::[P_{A1lacO-Tenth} MutaT7] |
| drApo1 − T7 | DH10B Δung ΔmotAB ΔcsgABCDEFG [P_{lacI}<>P_{tac}] |
| | Δ(araA-leu)7697::[P_{A1lacO-Tenth} drApo1 − T7] |
| MP6 | DH10B Δung ΔmotAB ΔcsgABCDEFG MP6(Cm^{R}) |
| MutaT7- | DH10B Δung [P_{lacI}<>P_{tac}] Δ(araA-leu)7697::[P_{A1lacO-Tenth} |
| csg⁺ mot⁺ | MutaT7] |

TABLE 6
Primer table. This table shows the primers used for lamda red
recombineering, restriction cloning of the terminator arrays, colony PCR
and Sanger sequencing.

| Primer Name | SEQ ID NO: | SEQUENCE |
|---|---|---|
| 5′ Ung | 29 | GCAGTTAAGCTAGGCGGATTGAAGATTCGCAGGAGAGCGAGATGGCT |
| kanccdB | | AACCCCTCATCAGTGCCAACATAGTAAG |
| 3′ Ung | 30 | AGCCGGGTGGCAACTCTGCCATCCGGCATTTCCCCGCAAATTTACTCA |
| kanccdB | | CTCCGCTCATTAGGCGGGC |
| delUng S | 31 | GCAGTTAAGCTAGGCGGATTGAAGATTCGCAGGAGAGCGAGATGGCT |
| | | AACAGTGAGTAAATTTGCGGGGAAATGCCGGATGGCAGAGTTGCCAC |
| | | CCGGCT |
| delUng AS | 32 | AGCCGGGTGGCAACTCTGCCATCCGGCATTTCCCCGCAAATTTACTCA |
| | | CTGTTAGCCATCTCGCTCTCCTGCGAATCTTCAATCCGCCTAGCTTAAC |
| | | TGC |
| 5′ | 33 | CGTTACTGGTTTCACATTCACCACCCTGAATTGACTCTCTTCCGGGCGC |
| pLacI: : kanccdB | | TCCCTCATCAGTGCCAACATAGTAAG |
| 3′ | 34 | TGGTGGCCGGAAGGCGAAGCGGCATGCATTTACGTTGACACCATCGAA |
| pLacI: : kanccdB | | TGCCGCTCATTAGGCGGGC |
| pLacI:pTacAS | 35 | ATTCACCACCCTGAATTGACTCTCTTCCGGGCGCTCATTATACGAGCCG |
| | | ATGATTAATTGTCAACATTCGATGGTGTCAACGTAAATGCATGCCGCTT |
| | | C |
| pLacI: : pTacS | 36 | GAAGCGGCATGCATTTACGTTGACACCATCGAATGTTGACAATTAATC |
| | | ATCGGCTCGTATAATGAGCGCCCGGAAGAGAGTCAATTCAGGGTGGTG |
| | | AAT |
| delcsgDF | 37 | TTTCGCTTAAACAGTAAAATGCCGGATGATAATTCCGGCTTTTTTATCT |
| | | GTTTGTTGAAATATCGGAATTAAAAAAGAATTCAAAAAAAAGCCCGC |
| delcsgDR | 38 | GCAGCAGACCATTCTCTCCAGATTCATCTTATGCTCGATATTTCAACAA |
| | | ACAGATAAAAAAGCCGGAATTAAAAAAGAATTCAAAAAAAAGCCCGC |
| delmotDF | 39 | CTTCATCAAAAAATGTCTGATAAAAATCGCTTATATCCATGCTCACGCT |
| | | GGACATCATCCTTCCAGAATTAAAAAAGAATTCAAAAAAAAGCCCGC |
| delmotDR | 40 | CGCCTGACGACTGAACATCCTGTCATGGTCAACAGTGGAAGGATGATG |
| | | TCCAGCGTGAGCATGGAGAATTAAAAAAGAATTCAAAAAAAAGCCCG |
| | | C |
| KanF- | 41 | GCTCGACGTTGTCACTGAAGC |
| AmilICP | | |
| AmilCP- | 42 | CGCCGTCGGGCATGC |
| KanR | | |
| 5′ | 43 | GTCGTCACTCTATCTGGCGTCACACCTCTCAGAACACCAACAAACACG |
| drApoI: : kanccdB | | TTCCGCTCATTAGGCGGGC |
| 3′ | 44 | TTCGGGCAGAAGTAACGTTCGGTGGTGAATTTTTCGATGAAGTTAACT |
| drApoI: : kanccdB | | TCCCTCATCAGTGCCAACATAGTAAG |
| drApoI S | 45 | GTCGTCACTCTATCTGGCGTCACACCTCTCAGAACACCAACAAACACG |
| | | TTCAAGTTAACTTCATCGAAAAATTCACCACCGAACGTTACTTCTGCCC |
| | | GAA |
| drApoI AS | 46 | TTCGGGCAGAAGTAACGTTCGGTGGTGAATTTTTCGATGAAGTTAACT |
| | | TGAACGTGTTTGTTGGTGTTCTGAGAGGTGTGACGCCAGATAGAGTGA |
| | | CGAC |
| dAraLeu7697 | 47 | CAGCAGCAGAACGCCCGGCACGTGCTCTGCCAGTTGTTCAATGGCCTG |
| kanccdB F | | ATCCCTCATCAGTGCCAACATAGTAAG |
| dAraLeu7697 | 48 | TGAGCAGGCAATCAGCAGTTGATAACCCCGTTGCCGCGCCTGGCGTTC |
| kanccdB R | | AACCGCTCATTAGGCGGGC |
| dAraLeu7697- | 49 | CAGCAGCAGAACGCCCGGCACGTGCTCTGCCAGTTGTTCAATGGCCTG |
| rApoI | | ATTCGAGTTTATGGCTAGCTCAGTCC |
| dAraLeu7697- | 50 | TGAGCAGGCAATCAGCAGTTGATAACCCCGTTGCCGCGCCTGGCGTTC |
| T7 | | AACTGAAAATCTTCTCTCATCCGCC |
| 5′ | 51 | GCAGAACGCCCGGCACGTGCTCTGCCAGTTGTTCAATGGCCTGATTCG |
| prApoI: : kanccdB | | AGCCGCTCATTAGGCGGGC |
| 3′ | 52 | CGACGACGCAGGGTCGGGTCAACCGCAACCGGACCGGTTTCAGAAGA |
| prApoI: : kanccdB | | CATCCCTCATCAGTGCCAACATAGTAAG |
| PA1lacO-1 F | 53 | GCAGAACGCCCGGCAC |
| PA1lacO-1 R | 54 | CGACGCAGGGTCGGG |
| pA1lacO1- | 55 | GCAGAACGCCCGGCACGTGCTCTGCCAGTTGTTCAATGGCCTGATTCG |
| HA Tenth | | AGAAAGAGTGTTTATTTGTGAGCGGATAACAATTACAATTAGATTCAA |
| gblock | | TTGTGAGCGGATAACAATTTCACACAGGCTAGCGAATTCGAGCTCCCT |
| | | CTAGAAATAATTTTGTTTAACTTTAAGAAGGAGATATACCATGGGCAG |
| | | CAGCTACCCATACGACGTACCAGATTACGCTATGTCTTCTGAAACCGG |
| | | TCCGGTTGCGGTTGACCCGACCCTGCGTCGTCG |
| NheI-UUCG- | 56 | CTAGCCAGCTTGGGTCTCCCTAGGTCGAGCTCCGTCGACCTAGCATAA |
| BamHI S | | CCCCGCGGGGCCTCTTCGGGGGTCTCGCGGGGTTTTTTGCTGAAAGG |
| NheI-UUCG- | 57 | GATCCCTTTCAGCAAAAAACCCCGCGAGACCCCCGAAGAGGCCCCGCG |
| BamHI AS | | GGGTTATGCTAGGTCGACGGAGCTCGACCTAGGGAGACCCAAGCTGG |
| 1493 | 58 | AAAAAAaagcttGTTGACAATTAATCATCGGCATAGTATATCGGCATAGT |
| | | ATAATACGACAAGGTGAGGAACTAAACCAcGGGATCGGCCATTGAAC |
| 1494 | 59 | AAAAAAttaattaaGCTCTAGAGAATTGATCCCCTCAG |
| 2165 | 60 | ttgacaattaatcatcggctcgAagcttG |
| 1197 | 61 | AAAAAAggatccTTCGCCATTCAGGCTGCG |

General recombineering: The E. coli genome was edited using seamless lambda red recombineering with ccdB counterselection as previously described (Wang et al., Nucleic Acids Res. 2014 March; 42(5): e37). Cells were first transformed with the temperature-sensitive psc101-gbaA recombineering plasmid and plated on LB agar with 10 μg/mL tetracycline and incubated for 24 hr at 30° C. Colonies were picked and grown in LB with 10 μg/mL tetracycline overnight at 30° C. (18-21 hrs). The overnights were diluted 25-fold in LB with 10 μg/mL tetracycline and grown at 30° C. for about 2 hours until they reached an OD₆₀₀ of 0.3-0.4. The ccdA antitoxin and recombineering machinery were then induced by adding arabinose and rhamnose to a final concentration of 2 mg/mL each and then growing the cultures at 37° C. for 40 minutes to an OD₆₀₀ of ˜0.6. The cultures were then placed on ice, washed twice with ice-cold sterile ddH₂O, resuspended in ˜25 μL of ice-cold sterile ddH₂O and electroporated with ˜200 ng of the appropriate kan-ccdB targeting cassette (1.8 kV, 5.8 ms, 0.1 cm cuvette, BioRad Micropulser). The cells were then recovered in SOC with 2 mg/mL arabinose at 30 C for 2 hours, then plated on LB agar plates with 50 μg/mL kanamycin and 2 mg/mL arabinose and incubated for 24 hours at 30° C. Colonies that appeared had incorporated the kan-ccdB targeting and were picked and grown in LB with 50 μg/mL kanamycin and 2 mg/mL arabinose at 30° C. overnight (18-21 hours). The cultures were then diluted 25-fold in LB with 50 μg/mL kanamycin and 2 mg/mL arabinose and grown at 30° C. for about 2 hours until they reached an OD₆₀₀ of 0.3-0.4. The recombineering machinery was then induced by adding rhamnose to a final concentration of 2 mg/mL each and then growing the cultures at 37° C. for 40 minutes to an OD₆₀₀ of ˜0.6. The cultures were then placed on ice, washed twice with ice-cold sterile ddH₂O, resuspended in ˜25 μL of ice-cold sterile ddH₂O and electroporated with ˜200 ng of the final targeting cassette that will replace the kan-ccdB cassette currently integrated in the genome (1.8 kV, 5.8 ms, 0.1 cm cuvette, BioRad Micropulser). The cells were then recovered in SOC with 2 mg/mL arabinose at 30 C for 2 hours, then were washed once with LB to remove the arabinose and cease production of the ccdA antitoxin. The cultures were then plated on LB agar plates at various dilutions with 100 μg/mL streptomycin and incubated for 24 hours at 37° C. Without the ccdA antitoxin, the ccdB toxin will kill cells that have not replaced the integrated kan-ccdB cassette with the final targeting cassette. The colonies that grow should have the final targeting cassette integrated, but were screened by PCR or sequencing to confirm final targeting cassette integration as some colonies simply have inactivated the ccdB toxin. Once a clone with the desired change was found, the temperature-sensitive psc101-gbaA recombineering plasmid was cured by plating on LB agar with 100 μg/mL streptomycin and incubating at 42° C. for 18-21 hours, then streaking a colony from the plate on LB agar with 100 μg/mL streptomycin and incubating at 42° C. for another 18-21 hours. The colonies from the second plate were grown in LB with 100 μg/mL streptomycin at 37° C. to be used or to make glycerol stocks. The colonies were also incubated in LB with 10 μg/mL tetracycline at 30° C. to ensure tetracycline sensitivity and confirm that the recombineering plasmid was cured.

ung Deletion: In order to prevent dU→dC repair and increase the mutagenesis rate, uracil DNA glycosylase (ung) was deleted in several of the strains used in this work (Duncan B. K., J. Bacteriol. 1985 November; 164(2): 689-95). Deletion of ung was accomplished through lambda red recombineering, using a kan-ccdB targeting cassette that was amplified from R6K-kan-ccdB using primers 5′ Ung kanccdB and 3′ Ung kanccdB. Once the kan-ccdB targeting cassette replaced the ung gene, the kan-ccdB cassette was deleted using the annealed oligos delUng S and delUng AS as the targeting cassette to generate a markerless ung deletion.

Increasing lacI expression: The expression of the lacI repressor in DH10B cells was increased by replacing the endogenous P_{lacI} promoter with the strong P_{tac} promoter using lambda red recombineering. A kan-ccdB targeting cassette was amplified from R6K-kan-ccdB using primers 5′ pLacI::kanccdB and 3′ pLacI::kanccdB and used to replace the endogenous P_{lacI} promoter with the kan-ccdB cassette. The kan-ccdB cassette was replaced with P_{tac} using the annealed oligos pLacI::pTac S and pLacI::pTac AS.

Deleting the motAB and csgABCDEFG operons to decrease biofilm formation: Deletions of the motAB operon (Pratt L. A. and Kolter R., Mol. Microbiol. 1998 October; 30(2): 285-93) and the csgABCDEFG (Prigent-Combaret et al., Environ. Microbiol. 2000 August; 2(4): 450-64) have been shown to produce strains of E. coli that are deficient in biofilm formation. To minimize inlet line contamination and clogs in bioreactor experiments due to biofilms, the motAB and csgABCDEFG operons were deleted using one-step DIRex lambda red recombineering (Nasvall J., PLoS One. 2017 Aug. 30; 12(8): e0184126). The motAB targeting half-cassettes were amplified from R6K-AmilCP-kan-ccdB using primers delmotDF and AmilCP-KanR and from R6K-kan-ccdB-AmilCP using primers delmotDR and KanF-Ami1CP. The csgABCDEFG targeting half-cassettes were amplified from R6K-AmilCP-kan-ccdB using primers delcsgDF and AmilCP-KanR and from R6K-kan-ccdB-AmilCP using primers delcsgDR and KanF-AmilCP. The motAB or csgABCDEFG half cassettes were co-electroporated to replace motAB or csgABCDEFG with a kan-ccdB cassette flanked by large AmilCP inverted repeats nested between short 30 bp direct repeats. The repeat architecture leads to a high rate of spontaneous excision that was selected for using ccdB counterselection to obtain markerless deletions of motAB and csgABCDEFG.

Deactivated rApo1: The E63Q mutant of rApo1 cytidine deaminase has been shown to be catalytically dead (Navaratnam et al., Cell. 1995 Apr. 21; 81(2): 187-95). Lambda red recombineering was used to generate strains with deactivated rApoI and deactivated rApoI−T7 using a kan-ccdB targeting cassette that was amplified from R6K-kan-ccdB using primers 5′ drApoI::kanccdB and 3′ drApoI::kanccdB. Once the kan-ccdB targeting cassette replaced the E63 codon, the kan-ccdB cassette was replaced with a glutamine codon using the annealed drApoI S and drApoI AS as the targeting cassette to generate an E63Q mutant.

Insertion of rApo1 and MutaT7 into the E. coli genome: rApo1 and MutaT7 were inserted into the genome at the seam of the large Δ(araA-leu)7697 deletion in DH10B E. coli using lambda red recombineering. A kan-ccdB targeting cassette was amplified from R6K-kan-ccdB using primers dAraLeu7697 kanccdB F and dAraLeu7697 kanccdB R and used to the insert the kan-ccdB cassette between 62,378 bp and 62,379 bp in the DH10B genome (Durfee et al., J. Bacteriol. 2008 April; 190(7): 2597-606). Then targeting cassettes containing rApo1 or MutaT7 were amplified from BBa_J23114_lacO rApo1 and BBa_J23114_lacO MutaT7, respectively, using primers dAraLeu7697-rApoI and dAraLeu7697-T7 and were used to replace kan-ccdB with rApoI or MutaT7.

Replacement of promoter BBa_J23114 with P_{AllacO-Tenth}: The BBa_J23114 promoter from the Anderson Collection (parts.igem.org/Promoters/Catalog/Anderson) that controlled the expression of rApo1 or MutaT7 from the DH10B genome was replaced with the promoter P_{AllacO-Tenth} which was intended to be a weaker version of the P_{AllacO} promoter (Camsund et al., J. Biol. Eng. 2014 Jan. 27; 8(1): 4). A kan-ccdB targeting cassette was amplified from R6K-kan-ccdB using primers 5′ prApoI::kanccdB and 3′ prApoI::kanccdB and used to replace BBa_J23114 with a kan-ccdB cassette. The kan-ccdB cassette was replaced with P_{AllacO-Tenth} using the targeting cassette amplified from the pAllacO-tenth gblock using primers PAllacO-1 F and PAllacO-1 R.

Mutation assay: To test mutagenesis rates, the control and mutagenic strains (Strep^{R}) carrying reporter plasmids (Amp^{R}) were streaked out on LB agar with 100 μg/mL streptomycin and 100 μg/mL ampicillin and grown at 37° C. for 24 hours. Then single colonies were picked in triplicate for each sample and grown in 5 mL LB with 100 μg/mL streptomycin,100 μg/mL ampicillin and 25 mM arabinose (with 10 μg/mL chloramphenicol if the strain contains MP6) at 37° C., 250 r.p.m. for 24 hours. Then 1 mL aliquots of each overnight were pelleted at 6000×g for 3 minutes and resuspended in 1 mL LB to remove arabinose. Then 50 μL of each resuspension was plated on LB agar plates with 50 μg/mL tetrazolium chloride and 200 μg/mL kanamycin, 20 μg/mL tetracycline, 100 μg/mL fosfomycin or 100 μg/mL rifampicin unless otherwise stated. 50 μL of a 100,000-fold dilution of each culture was also plated on LB agar with 100 μg/mL streptomycin, 100 μg/mL ampicillin and 50 μg/mL tetrazolium chloride. After incubating the plates at 37° C. for 48 hours, they were imaged by inverting the plates onto transparencies and scanning on a document scanner at a resolution of 400 d.p.i. The colonies were then counted using the software OpenCFU (3.9.0) (Geissmann Q., PLoS One. 2013; 8(2): e54072), with minimum colony radius set to 3, the maximum colony radius set to 50 and the regular threshold set to 4.

Chemical mutagens: Mutagenesis with ethane methyl sulfonate (EMS) was performed as previously described (Cupples C. G. and Miller J. H., Proc. Natl. Acad. Sci. U.S.A. 1989 July; 86(14): 5345-49). An overnight culture of each sample was subcultured and grown until it reached a density of 2-3×10⁸ cells per mL (log phase). 5 mL aliquots of cells were chilled on ice, washed twice with sodium phosphate buffer (pH 7) and resuspended in 1 mL of 1× PBS in a 1.5 mL eppendorf tube. EMS was added while cold by pipetting 14 μL of EMS into 1 ml of resuspended cells. Eppendorfs were sealed, and mixed at 1000 r.p.m. for 60 minutes at 37° C. The cells were then washed twice with LB, and resuspended in 1 mL of LB. Immediately after washing, a viability measurement was performed by plating 50 μL of a 10,000-fold dilution of mutagen and mock-treated cultures on LB agar with 100 μg/mL streptomycin, 100 μg/mL ampicillin and 50 μg/mL tetrazolium chloride. For mutation rate assessment, 500 μL of each resuspension were inoculated into 5 ml of LB with 100 μg/mL streptomycin and 100 μg/mL ampicillin. The cultures were grown at 37° C. for 20 hours, then 50 μL of each culture was plated on LB agar 50 μg/mL tetrazolium chloride and 100 μg/mL rifampicin. 50 μL of a 100,000-fold dilution of each culture was also plated on LB agar with 100 μg/mL streptomycin, 100 μg/mL ampicillin and 50 μg/mL tetrazolium chloride. After 48 hours of incubation, plates were imaged on a document scanner at a resolution of 400 d.p.i, and colonies were subsequently counted using the software OpenCFU (3.9.0) (Geissmann Q., PLoS One. 2013; 8(2): e54072), with minimum colony radius set to 3, the maximum colony radius set to 50 and the regular threshold set to 4.

Continuous culture of T7 promoter+antisense T7 promoter reporter plasmid and sequencing: The T7 promoter+antisense T7 promoter reporter plasmid was continuously cultured in the MutaT7-csg⁺ mot⁺ strain in a 70 mL culture in a round-bottomed flask that was slowly stirred in a 37° C. mineral oil bath. The culture was aerated through a needle that was connected to a standard aquarium pump and LB with 100 μg/mL streptomycin, 100 μg/mL ampicillin and 0.5% isopropanol (as antifoaming agent) was fed into the culture via a needle connected to a peristaltic pump at a rate of ˜0.5 volumes/hour. Fractions were collected every 3 days for 12 days. Each fraction was plated for single colonies on LB agar with 100 μg/mL ampicillin and 10 clones from each fraction were Sanger sequenced by colony PCR with primers 1493 and 1494.

Continuous culture of T7 promoter+filler DNA and T7 promoter+terminators reporter plasmids and sequencing: The T7 promoter+filler DNA and T7 promoter+terminators reporter plasmids were continuously cultured in the Δung (negative control), MutaT7 and MP6 strains in 20 mL cultures in a previously described multiplex bioreactor setup (Miller et al., J. Vis. Exp. 2013 Feb. 23; (72): e50262). The reactor was stored in a 37° C. warm room and was aerated and stirred with aquarium pumps. LB with 100 μg/mL streptomycin, 100 μg/mL ampicillin, 100 μg/mL cycloheximide, 0.01% (v/v) antifoam 204 and 150 μg/mL arabinose (+10 μg/mL chloramphenicol in the case of the MP6 strain) was pumped into each reaction vessel at a rate of 0.87 volumes/hour. Fractions were collected every 3 days. Each fraction was plated on LB agar with 100 μg/mL streptomycin and 100 μg/mL ampicillin and 12 single colonies from each plate were grown in 5 mL LB with 100 μg/mL ampicillin. DNA was isolated from each overnight using the Qiaprep 96 Turbo Miniprep Kit and quantified using PicoGreen assay. 1 ng of each sample was prepared using the Illumina NexteraXT Sample Preparation kit. Samples were barcoded and pooled prior to sequencing on an Illumina MiSeq 300v2 cartridge to obtain 2×150 base pair paired-end reads. Sequencing reads were aligned against respective plasmid sequences using bwa mem 0.7.10-r789 [RRID:SCR_010910]. Allele pileups were generated using samtools v.0.1.19 mpileup [RRID:SCR_002105] with flags-d 10000000-excl-flags 2052, and allele counts/frequencies were extracted (Li H., Bioinformatics. 2011 Nov. 1; 27(21): 2987-93; Li et al., Bioinformatics. 2009 Aug. 15; 25(16): 2078-79). Only positions with greater than 10-fold coverage in all replicates of each sample were included in the analysis. Fixed variant alleles (present at greater than 85% frequency) for each sample are reported. Sanger sequencing was also performed on a PCR amplicon from 96 clones of Δung (negative control) and MutaT7 after 15 days of continuous culture carrying T7 promoter+terminators reporter plasmid. Primers 2165 and 1197 were used to amplify and Sanger sequence the Kan^{R} gene.

### Example 6

### Directed Mutagenesis

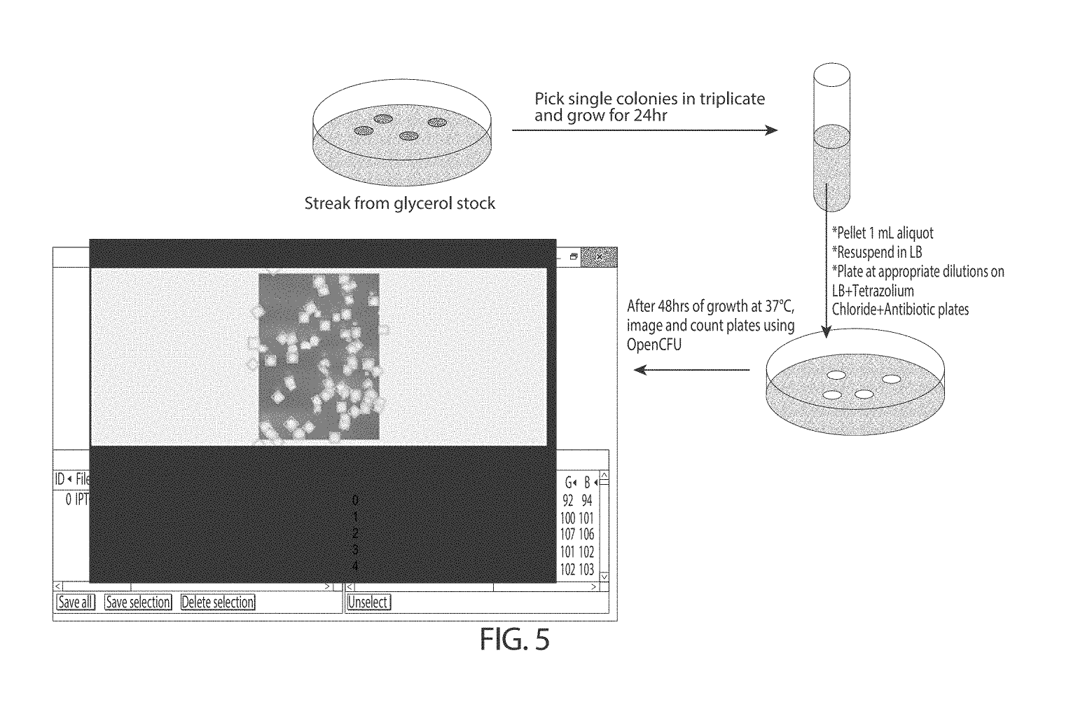

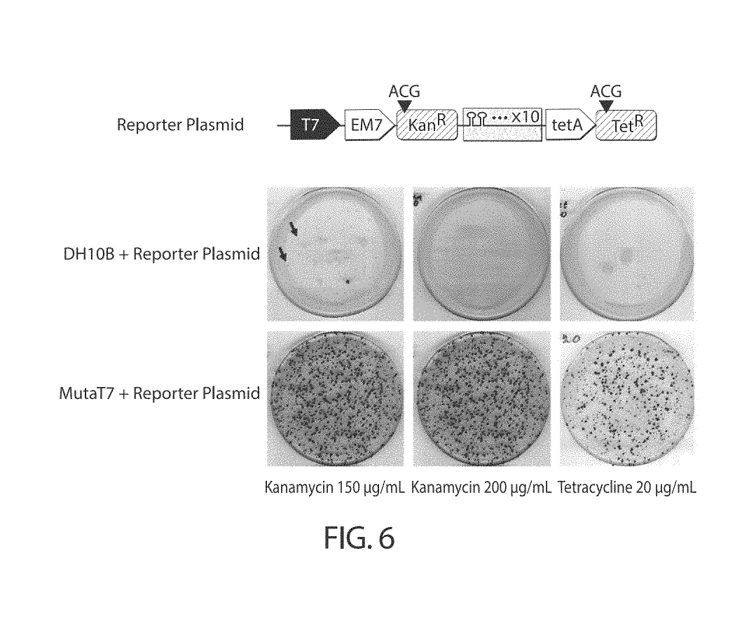

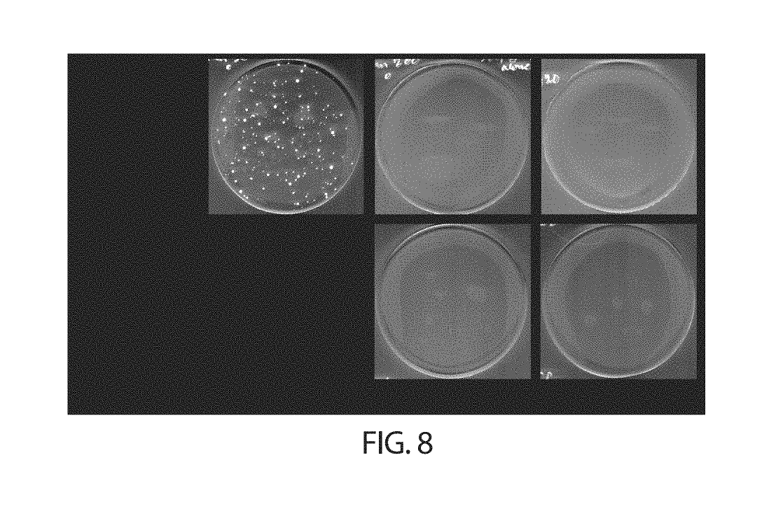

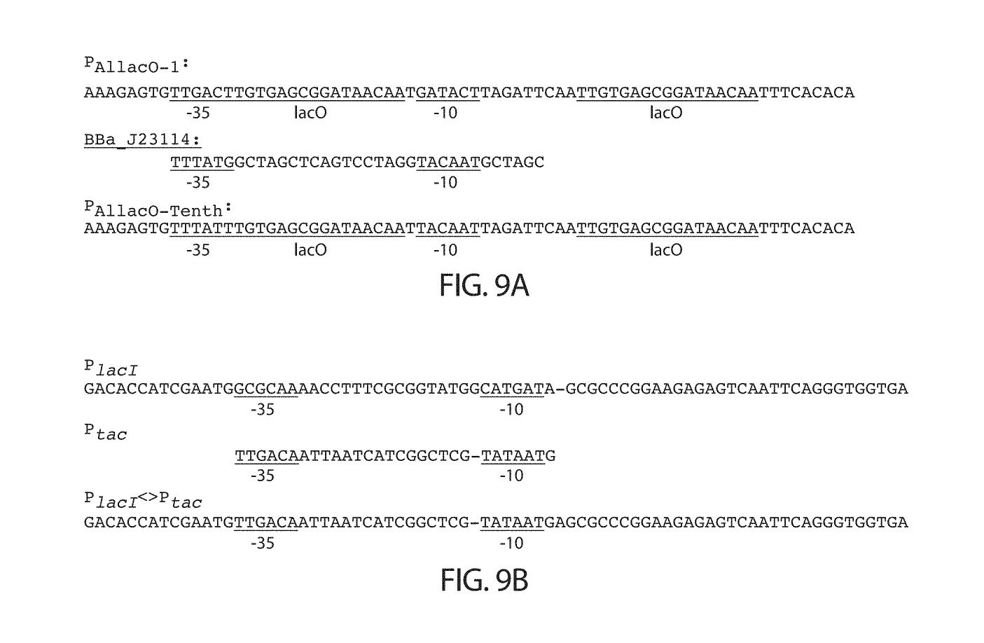

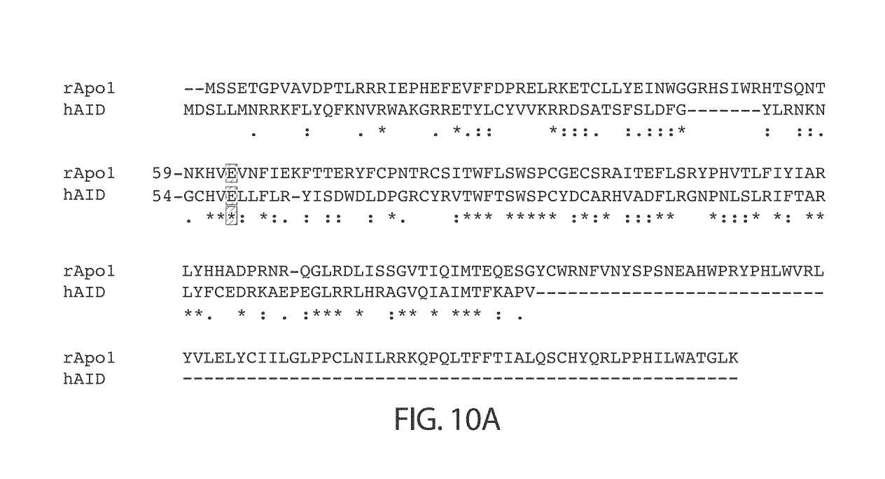

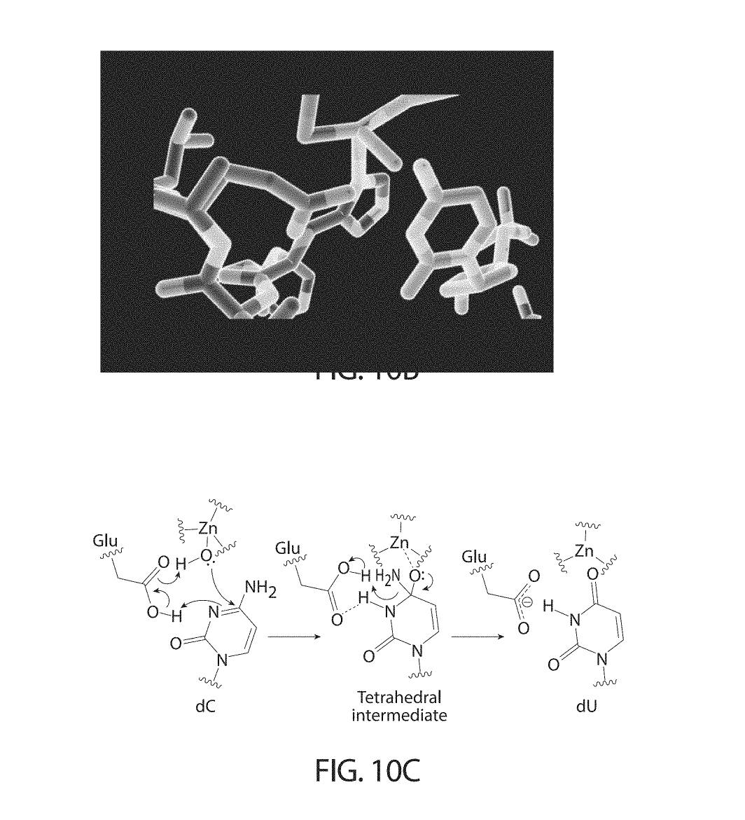

Monomeric RNA polymerases possess inherently high promoter specificity (Rong et al., J. Biol. Chem. 1998 Apr. 24; 273(17): 10253-60) and high processivity during transcription (Thiel et al., J. Gen. Virol. 2001 June; 82(6): 1273-81). Cytidine deaminases are potent DNA-damaging enzymes that act on ssDNA substrates formed during transcription (Thiel et al., J. Gen. Virol. 2001 June; 82(6): 1273-81; Ramiro et al., Nat. Immunol. 2003 May; 4(5): 452-56). It was envisioned that merging the unique features of these two enzyme classes by creating a fusion “mutaT7” protein consisting of a cytidine deaminase (rApo1) fused to T7 RNA polymerase (T7-pol) would facilitate the targeting of mutations to any DNA region lying downstream of a T7 promoter (FIG. 1B). Thus, rApo1 was fused to the N-terminus of T7-pol because the carboxy group of the T7-pol C-terminus is implicated in catalysis during the elongation phase (Lykke-Andersen J. and Christiansen J., Nucleic Acids Res. 1998 Dec. 15; 26(24): 5630-35). As preliminary overexpression appeared to be toxic, reduced expression of mutaT7 was sought by reducing promoter strength and subsequently minimizing copy number via integration into the E. coli genome with seamless recombineering (FIG. 9, TABLE 5, and TABLE 6). Targeted mutagenesis was assayed using a codon reversion assay based on bacteria artificial chromosome reporter plasmids either having or lacking a T7 promoter sequence upstream of silent drug resistance genes with ACG triplets in place of their ATG start codons (FIG. 1C, FIG. 5, FIG. 6, and FIG. 8). The kanamycin (Kan) resistance gene Kan^{R} was placed immediately downstream of the T7 promoter. In this assay, successful C→T mutagenesis at the start codon yields Kan-resistant colonies. Global mutagens such as MP6 yielded high levels of Kan-resistant colonies regardless of the presence or absence of a T7 promoter, consistent with a lack of targeting. In contrast, significant Kan resistance was only observed in mutaT7 strains with reporter plasmids having a T7 promoter upstream of the Kan^{R} gene, indicating successful targeted mutagenesis. Importantly, expression of a catalytically dead version of mutaT7 (drApo1−T7) lacking a critical residue for cytidine deaminase activity (Harris et al., Mol. Cell. 2002 November; 10(5): 1247-53) yielded Kan resistance frequencies similar to background levels, indicating that T7 activity was not responsible for increased Kan resistance (FIGS. 10A-10C).

T7 promoter-dependent Kan^{R} mutagenesis by mutaT7 shows that one can target mutagenesis to a desired DNA region. Since T7-pol is highly processive, it was anticipated mutations would also be introduced further downstream of the T7 promoter. The presence in the reporter plasmid of a tetracycline-resistance (Tet^{R}) gene with an inactive, ACG start codon separated by an ˜1.6 kbp spacer DNA from the Kan^{R} gene provided a mechanism to assay such processivity. High levels of mutaT7-dependent Tet resistance was observed only in reporter strains having the T7 promoter, consistent with targeting and processive introduction of mutations across a lengthy DNA region. Once again, global mutagens generated Tet-resistant colonies in all reporter plasmids.

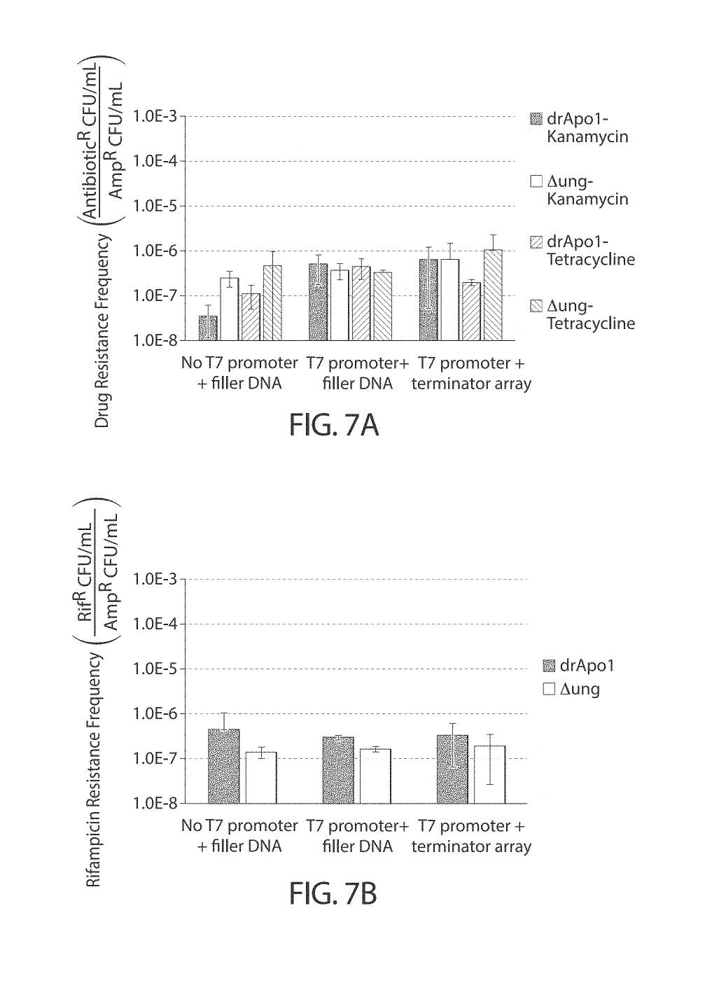

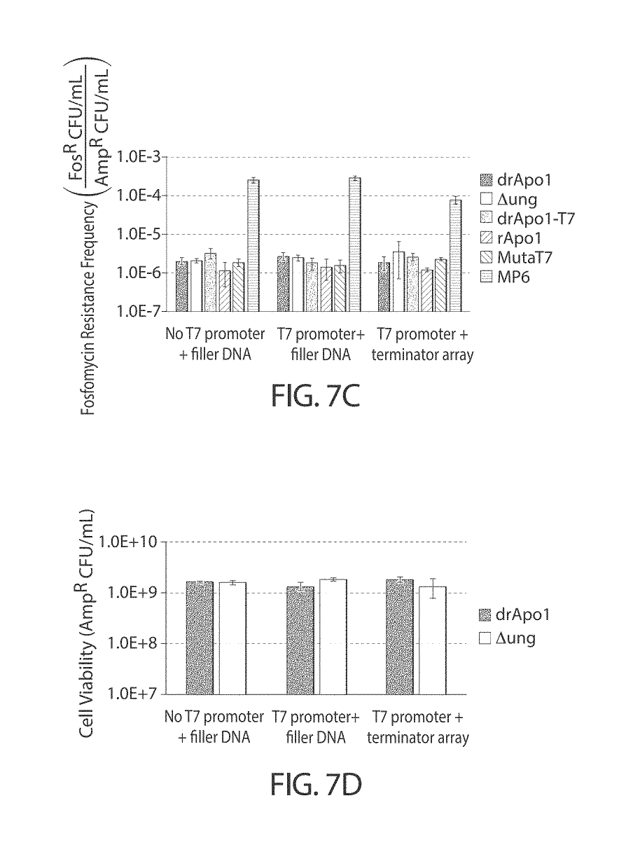

Targeted mutagenesis using the processive mutaT7 chimera requires not just recruitment to a DNA locus but also termination upon reaching the end of the DNA region of interest. To address termination, Kan^{R}/Tet^{R} reporter plasmids were used in which the DNA spacer was replaced with one or more T7 terminators and then assayed for both Kan and Tet resistance. Four or more copies of the T7 terminator was sufficient to prevent mutagenesis beyond the DNA of interest (FIGS. 4A-4B). Using the T7 terminator array, Tet resistance was observed for mutaT7 strains similar to background levels, while Kan resistance remained high. Global mutagens again induced high levels of Kan- and Tet-resistance, regardless of the presence of a T7 terminator array (TABLE 7). To further assess whether the MutaT7 chimera induces mutagenesis only on the target DNA of interest, the emergence of bacterial colonies resistant to rifampicin was evaluated (Garibyan et al., DNA Repair. 2003 May; 2(5): 593-8) and fosfomycin (Nilsson A. I., Antimicrob. Agents Chemother. 2003 September; 47(9): 2850-58). Because resistance to these two drugs can emerge by multiple mutations in the genome, the appearance of resistant colonies correlates with off-target mutation rates in the genome (Badran A. H. and Liu D. R., Curr. Opin. Chem. Biol. 2015 February; 24: 1-10; Garibyan et al., DNA Repair. 2003 May; 2(5): 593-8), in analogy to the emergence of “cheaters” in directed evolution schemes. Growth of E. coli on either rifampicin- or fosfomycin-treated plates revealed that mutaT7-expressing samples displayed drug resistance frequencies comparable to background levels, as opposed to the high frequencies of antibiotic resistance which appeared in all global mutagenesis samples (FIG. 1E and FIGS. 7A-7D).

An important advantage of targeted mutagenesis is the ability to attain much larger viable library sizes by avoiding off-target, toxic mutations in essential genes outside the DNA region of interest. Based on the apparently low off-target mutagenesis rate of mutaT7, one might expect that E. coli carrying mutaT7 would have significantly higher viability than bacteria treated with global mutagens. Indeed, consistent with prior work (Badran A. H. and Liu D. R., Curr. Opin. Chem. Biol. 2015 February; 24: 1-10), very low viability was observed in all populations treated with global mutagens, whereas populations expressing mutaT7 possessed viability similar to untreated cells (FIG. 1F). It was also found that the total number of Kan-resistant colonies was similar between mutaT7 and globally mutagenized samples (FIG. 1G) despite the relatively lower mutagenesis rate (FIG. 1C), highlighting the beneficial effect that minimizing off-target mutations has on library size for in vivo evolution schemes.

TABLE 7
Mutation assay data. This table shows the antibiotic resistant
CFU/mL and frequencies obtained in the mutation assay.

| Strain | Reporter Plasmid | Resistance | CFU/mL | Std | Frequency | Std |
|---|---|---|---|---|---|---|
| drApoI | No T7 promoter + | Kan | 6.0E+1 | 4.0E+1 | 3.8E−8 | 2.6E−8 |
| | filler DNA | Tet | 1.8E+2 | 9.2E+1 | 1.1E−7 | 6.3E−8 |
| | | Fos | 3.3E+3 | 5.3E+2 | 2.0E−6 | 4.2E−7 |
| | | Rif | 7.6E+2 | 1.0E+3 | 4.5E−7 | 5.9E−7 |
| | | Amp | 1.6E+9 | 1.0E+8 | | |
| | T7 promoter + filler | Kan | 6.5E+2 | 3.9E+2 | 5.2E−7 | 3.4E−7 |
| | DNA | Tet | 6.3E+2 | 3.9E+2 | 4.6E−7 | 2.3E−7 |
| | | Fos | 3.5E+3 | 4.2E+1 | 2.7E−6 | 5.8E−7 |
| | | Rif | 4.0E+2 | 1.1E+2 | 3.0E−7 | 3.0E−8 |
| | | Amp | 1.3E+9 | 2.5E+8 | | |
| | T7 promoter + | Kan | 1.2E+3 | 1.2E+3 | 6.6E−7 | 6.1E−7 |
| | terminator array | Tet | 3.7E+2 | 2.3E+1 | 2.1E−7 | 3.6E−8 |
| | | Fos | 3.4E+3 | 1.3E+3 | 1.9E−6 | 7.4E−7 |
| | | Rif | 5.9E+2 | 4.2E+2 | 3.4E−7 | 2.7E−7 |
| | | Amp | 1.8E+9 | 2.0E+8 | | |
| Δung | No T7 promoter + | Kan | 4.1E+2 | 1.2E+2 | 2.6E−7 | 9.8E−8 |
| | filler DNA | Tet | 8.1E+2 | 8.6E+2 | 4.9E−7 | 4.8E−7 |
| | | Fos | 3.3E+3 | 5.2E+2 | 2.1E−6 | 3.0E−7 |
| | | Rif | 2.3E+2 | 8.1E+1 | 1.4E−7 | 3.8E−8 |
| | | Amp | 1.6E+9 | 1.4E+8 | | |
| | T7 promoter + filler | Kan | 6.9E+2 | 2.5E+2 | 3.8E−7 | 1.5E−7 |
| | DNA | Tet | 6.3E+2 | 2.3E+1 | 3.4E−7 | 2.1E−8 |
| | | Fos | 4.5E+3 | 6.6E+2 | 2.5E−6 | 4.9E−7 |
| | | Rif | 3.0E+2 | 2.0E+1 | 1.7E−7 | 2.0E−8 |
| | | Amp | 1.8E+9 | 1.2E+8 | | |
| | T7 promoter + | Kan | 5.9E+2 | 4.8E+2 | 6.7E−7 | 8.2E−7 |
| | terminator array | Tet | 9.2E+2 | 7.8E+2 | 1.0E−6 | 1.3E−6 |
| | | Fos | 3.7E+3 | 1.0E+3 | 3.6E−6 | 2.8E−6 |
| | | Rif | 2.0E+2 | 1.0E+2 | 1.9E−7 | 1.6E−7 |
| | | Amp | 1.3E+9 | 5.6E+8 | | |
| drApoI-T7 | No T7 promoter + | Kan | 1.1E+2 | 4.2E+1 | 1.2E−7 | 3.3E−8 |
| | filler DNA | Tet | 2.2E+2 | 7.2E+1 | 2.6E−7 | 6.1E−8 |
| | | Fos | 2.6E+3 | 2.3E+2 | 3.3E−6 | 1.0E−6 |
| | | Rif | 1.7E+2 | 1.2E+2 | 2.2E−7 | 1.8E−7 |
| | | Amp | 8.7E+8 | 3.2E+8 | | |
| | T7 promoter + filler | Kan | 2.3E+2 | 1.2E+2 | 1.2E−7 | 3.9E−8 |
| | DNA | Tet | 5.9E+2 | 3.4E+2 | 3.9E−7 | 3.2E−7 |
| | | Fos | 3.0E+3 | 2.1E+2 | 1.8E−6 | 6.5E−7 |
| | | Rif | 2.6E+2 | 8.7E+1 | 1.5E−7 | 3.8E−8 |
| | | Amp | 1.8E+9 | 6.5E+8 | | |
| | T7 promoter + | Kan | 1.5E+2 | 5.0E+1 | 1.3E−7 | 8.2E−8 |
| | terminator array | Tet | 3.7E+2 | 1.2E+1 | 3.1E−7 | 9.8E−8 |
| | | Fos | 3.3E+3 | 7.9E+2 | 2.6E−6 | 5.7E−7 |
| | | Rif | 1.6E+2 | 2.0E+1 | 1.3E−7 | 2.3E−8 |
| | | Amp | 1.3E+9 | 3.2E+8 | | |
| MutaT7 | No T7 promoter + | Kan | 1.7E+2 | 7.6E+1 | 1.2E−7 | 6.1E−8 |
| | filler DNA | Tet | 2.5E+2 | 2.4E+2 | 1.6E−7 | 1.4E−7 |
| | | Fos | 2.8E+3 | 8.7E+2 | 1.9E−6 | 3.8E−7 |
| | | Rif | 5.5E+2 | 1.0E+2 | 3.7E−7 | 5.2E−8 |
| | | Amp | 1.5E+9 | 1.8E+8 | | |
| | T7 promoter + filler | Kan | 1.1E+4 | 3.1E+3 | 7.2E−6 | 2.9E−6 |
| | DNA | Tet | 3.1E+4 | 2.3E+3 | 2.0E−5 | 4.2E−6 |
| | | Fos | 2.5E+3 | 1.1E+3 | 1.6E−6 | 6.3E−7 |
| | | Rif | 4.2E+2 | 1.2E+2 | 2.6E−7 | 1.5E−8 |
| | | Amp | 1.6E+9 | 3.6E+8 | | |
| | T7 promoter + | Kan | 9.8E+3 | 1.9E+3 | 6.1E−6 | 5.5E−7 |
| | terminator array | Tet | 5.0E+2 | 2.4E+2 | 3.0E−7 | 1.2E−7 |
| | | Fos | 3.7E+3 | 6.3E+2 | 2.3E−6 | 2.4E−7 |
| | | Rif | 9.1E+2 | 5.1E+2 | 5.6E−7 | 2.7E−7 |
| | | Amp | 1.6E+9 | 2.1E+8 | | |
| MP6 | No T7 promoter + | Kan | 2.9E+3 | 4.7E+2 | 4.8E−5 | 1.6E−6 |
| | filler DNA | Tet | 3.5E+3 | 5.6E+2 | 5.9E−5 | 8.5E−6 |
| | | Fos | 1.5E+4 | 1.9E+3 | 2.5E−4 | 3.9E−5 |
| | | Rif | 9.5E+3 | 2.9E+3 | 1.5E−4 | 3.1E−5 |
| | | Amp | 6.0E+7 | 8.2E+6 | | |
| | T7 promoter + filler | Kan | 3.3E+3 | 4.6E+2 | 8.0E−5 | 2.8E−5 |
| | DNA | Tet | 4.8E+3 | 7.8E+2 | 1.1E−4 | 1.2E−5 |
| | | Fos | 1.2E+4 | 1.7E+3 | 2.9E−4 | 3.5E−5 |
| | | Rif | 9.3E+3 | 1.7E+3 | 2.2E−4 | 4.9E−5 |
| | | Amp | 4.4E+7 | 1.2E+7 | | |
| | T7 promoter + | Kan | 9.9E+3 | 1.2E+3 | 3.4E−5 | 1.0E−5 |
| | terminator array | Tet | 1.5E+4 | 3.6E+3 | 5.3E−5 | 2.0E−5 |
| | | Fos | 2.2E+4 | 1.3E+3 | 7.7E−5 | 1.8E−5 |
| | | Rif | 2.2E+4 | 3.4E+3 | 7.5E−5 | 2.5E−5 |
| | | Amp | 3.0E+8 | 6.6E+7 | | |

### Example 7

### Characterization of On-Target Versus Off-Target Mutagenesis

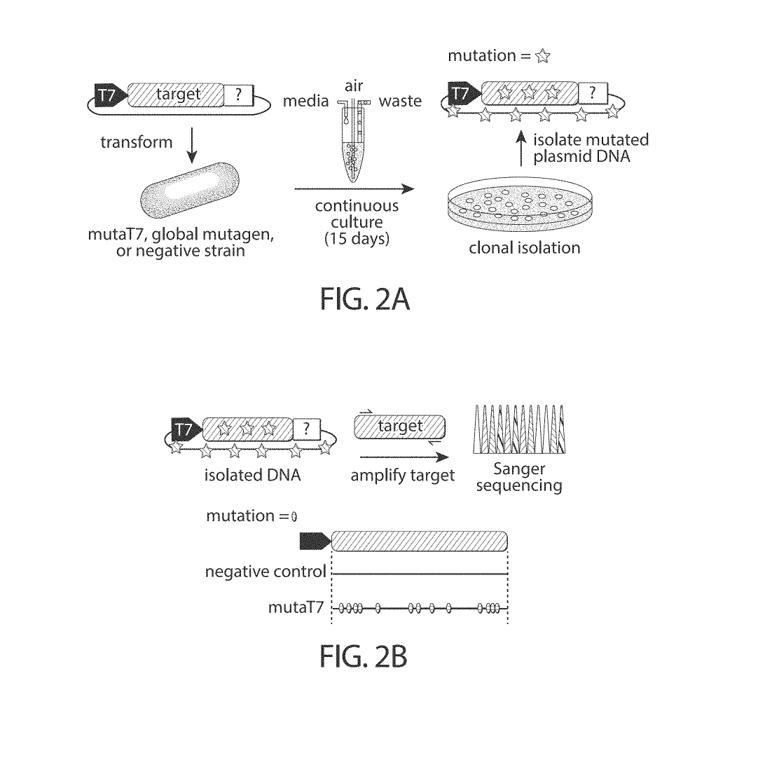

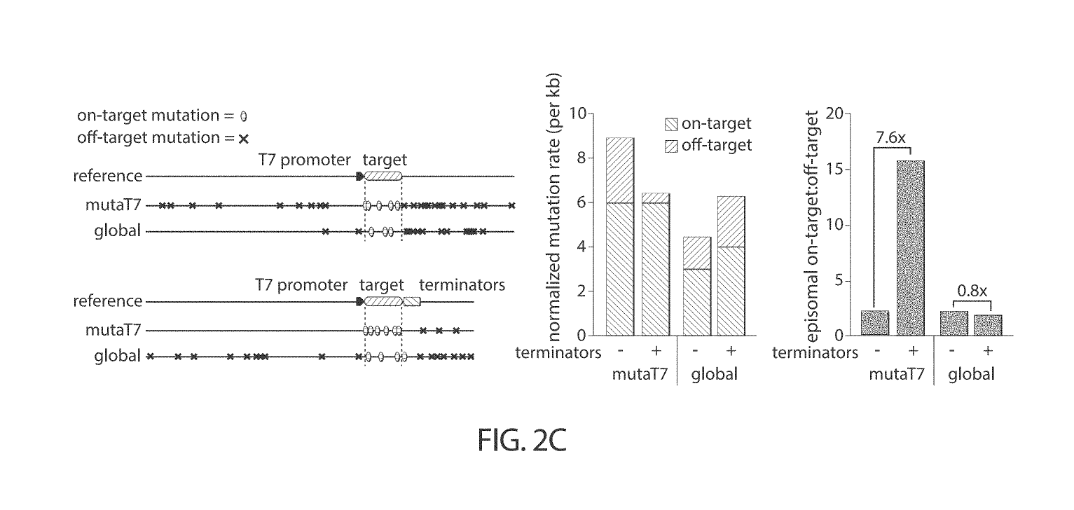

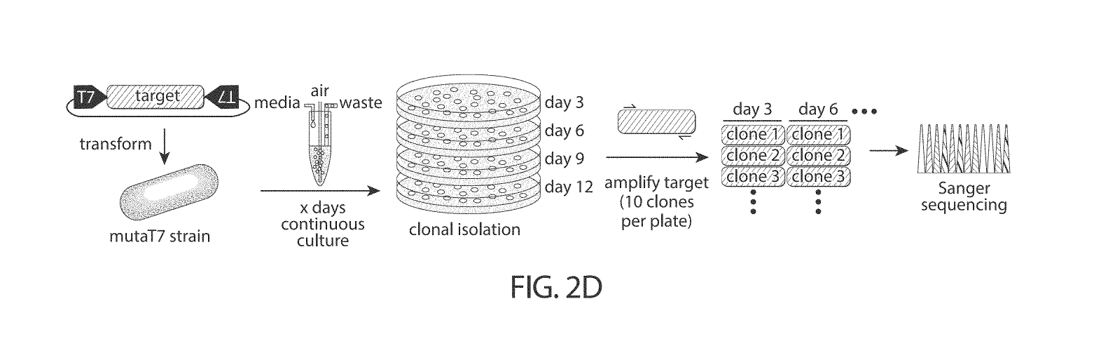

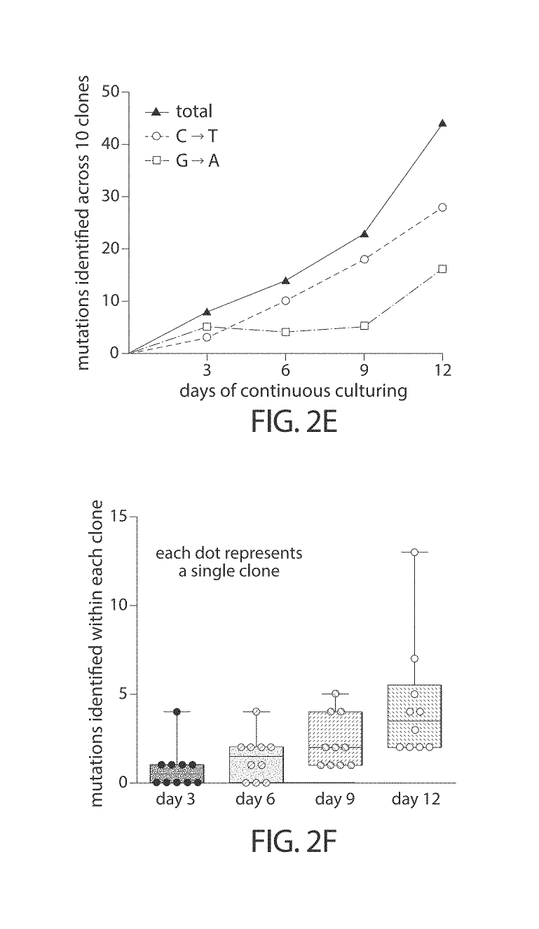

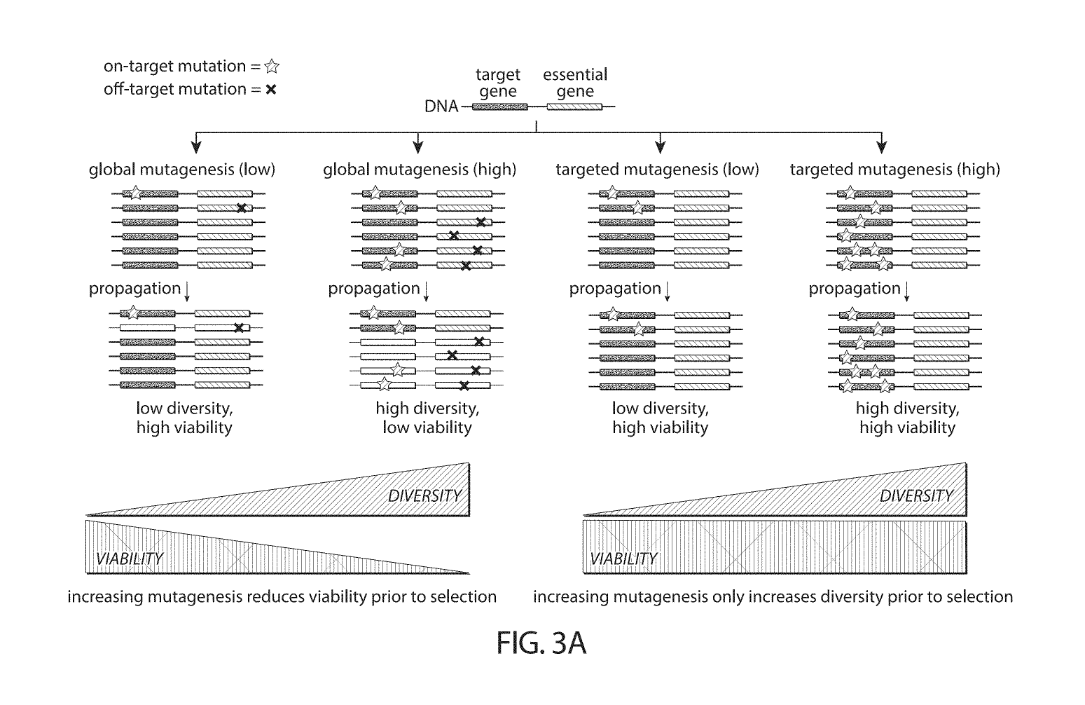

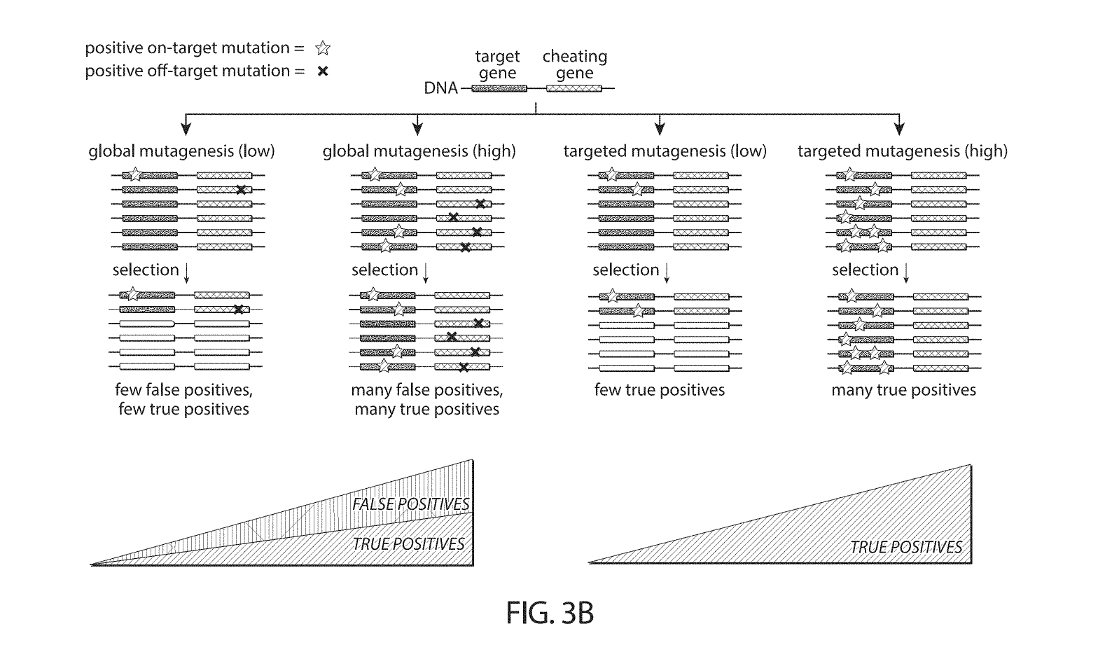

The assays in FIGS. 1A-1G show that mutaT7 targets mutations specifically to genes downstream of a T7 promoter and that the region of mutagenesis can be constrained by a terminator array. DNA sequencing was then used to better understand the processivity of mutagenesis and the extent of on-target versus off-target mutagenesis. An E. coli population expressing mutaT7 and an episomally expressed Kan^{R}/Tet^{R} reporter plasmid was allowed to drift in the absence of selection pressure for 15 days followed by Sanger sequencing of the Kan^{R} gene. Consistent with the expected processivity of mutaT7, mutations were found at multiple sites across the entire span of the Kan^{R} target gene independent of selection pressure (FIG. 2A).

Next, next generation sequencing was performed of the entire episomal reporter plasmid DNA sequence of 36 clones drawn from the same E. coli population as in FIG. 2A to directly assess on-target versus off-target mutagenesis across a ˜10 kb stretch of DNA containing only ˜1 kb of intended target DNA. The same approach was used to assess mutagenesis in a control E. coli population not treated with any mutagen and a population subjected to global mutagenesis. The clones drawn from mutaT7 samples displayed many more mutations throughout the plasmid when the terminator array was removed but the T7 promoter was maintained (FIG. 2B). Treatment with the MP6 global mutagen also led to mutations across the entire episome. In contrast, off-target mutations in mutaT7 samples appeared almost exclusively within the Kan^{R} target gene when both a promoter and terminator array were present, even after 15 days of continuous culturing, with off-target mutations present only to the same extent as in the control sample not treated with any mutagen. Additionally, the number of mutations in the target gene across different clones increases in frequency and position as cultures continuously grow without selection pressure (FIG. 2C). This observation suggests that, in contrast to the use of other genetic methods for global mutagenesis where the organism generally identifies a mechanism to shutdown mutagen expression, the high on-target to off-target mutation ration of mutaT7 is enabling long-term maintenance of expression in cells.

One disadvantage of mutaT7 is its limited mutational spectrum consistent with the use of a cytidine deaminase as the mutagenic component. Indeed, the sequencing results described above indicate that C to T transitions are exclusively obtained in the sense strand of targeted DNA using a single T7 promoter. It was hypothesized that the mutational spectrum could be doubled by installing a second T7 promoter that would recruit mutaT7 to the 3′-end of the DNA of interest. Installation of an antisense T7 promoter leads to the appearance of both G to A and C to T transitions throughout the target gene (FIG. 2D).

In summary, the processively acting mutaT7 chimera is capable of selectively targeting mutations to large, yet well-defined, regions of DNA in a living system with minimal human intervention. Moreover, the availability of T7 variants with altered transcription rates (Bonner et al., J. Biol. Chem. 1994 Oct. 7; 269(40): 25120-28) likely provides the opportunity to fine-tune mutation rates. Utilizing other base editing enzymes in place of cytidine deaminase, such as the adenosine deaminases (Gaudelli et al., Nature. 2017 Nov. 23; 551(7681): 464-71), can significantly widen the mutational spectrum of mutaT7 and further enable the creation of rich, diverse DNA libraries in vivo with minimal off-target effects. The ubiquitous applicability and high specificity of T7 RNA polymerase in a large number of diverse organisms (Lieber et al., Eur. J. Biochem., 1998 Oct. 1; 217(1): 387-94) will enable implementation of targeted mutagenesis in a broad range of evolutionary and synthetic biology settings.

### Example 8

### Materials and Methods for Examples 9-12

General: All PCR reactions for restriction cloning and recombineering targeting cassettes were performed using Q5 High Fidelity DNA Polymerase (New England Biolabs). All colony PCR reactions for sequencing were performed using OneTaq Quick-Load 2× Master Mix with Standard Buffer (New England Biolabs). Primers were obtained from Life Technologies. Gene blocks were obtained from Integrated DNA Technologies.

Reagents: The following reagents were obtained as indicated: Kanamycin monosulfate, fosfomycin, agar, and chloramphenicol (Alfa Aesar J61272, J66602, A10752, and B20841, respectively); tetracycline hydrochloride (CalBioChem 58346); rifampicin (TCI R0079); ampicillin (Fisher Bioreagents BP1760-25); streptomycin sulfate (MP Biomedical 100556); tetrazolium chloride, L-rhamnose, antifoam-204, and ethyl methanesulfonate (Sigma-Aldrich T8877, W373011, A8311, and M0880, respectively); L-arabinose and cycloheximide (Chem-Impex 01654 and 00083, respectively); and lysogeny broth (LB; Difco 244620); anhydrous sodium phosphate dibasic and monobasic sodium phosphate (Mallinckrodt 7917 and 7892, respectively); potassium chloride and isopropyl β-D-1-thiogalactopyranoside (Sigma P9333 and 16758, respectively); magnesium sulfate (Macron 6070-12); o-Nitrophenyl-β-galactoside and egg-white lysozyme (VWR 0789 and 0663, respectively); PopCulture lysis reagent (EMD Millipore 71092-4); 2-mercaptoethanol (Bio-Rad 161-0710); trimethoprim (Matrix Scientific 058373).

Cloning and Recombineering: All plasmids were generated by restriction cloning. Ligation reactions were performed using Quick Ligase (New England Biolabs). All DNA cloning was performed in DH10B cells (Invitrogen). The rApo1 gene was amplified from pET28b-BE1 (Komer et al., Nature. 2016 May 19; 533(7603): 420-2) and the T7 RNA polymerase gene was amplified from pTara (Wycuff and Matthews, Anal. Biochem. 2000 Jan. 1; 277(1): 67-73). Mutation assay reporter plasmids utilizing the single-copy BAC origin and the terminator arrays of the UUCG-T7 derivative of the T7 terminator (Mairhofer et al., ACS Synth. Biol. 2015 Mar. 20; 4(3): 265-73) were generated by serial insertion of the annealed oligos NheI-UUCG-BamHI S and NheI-UUCG-BamHI AS (TABLE 10). The folA gene was amplified from DH10B genomic DNA. All E. coli strains used in this work were engineered using lambda red recombineering strategies described in detail below.

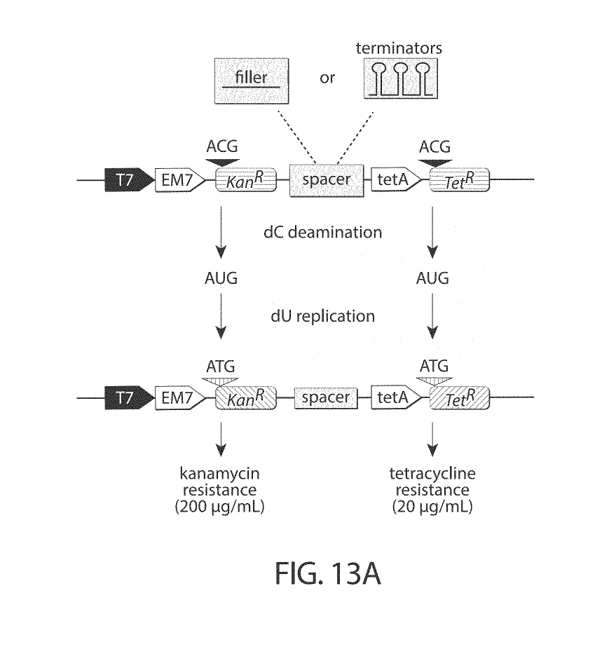

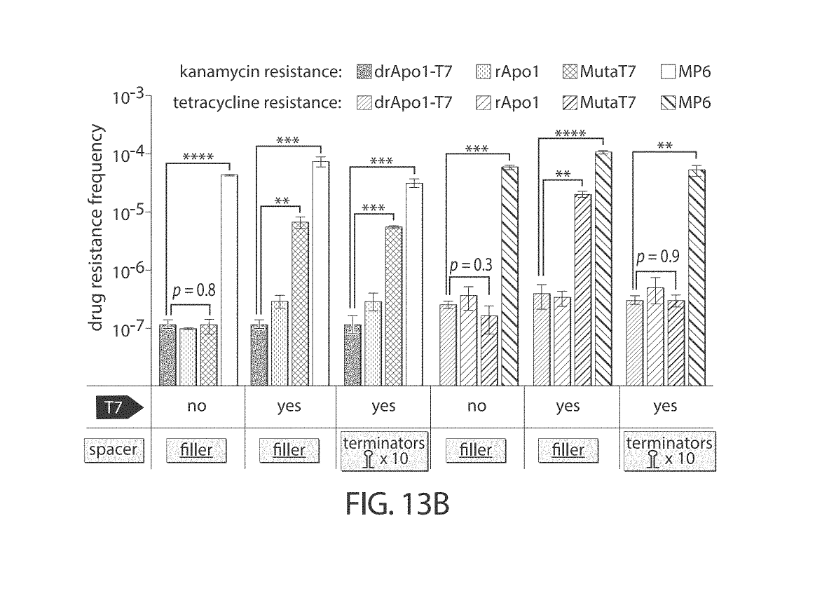

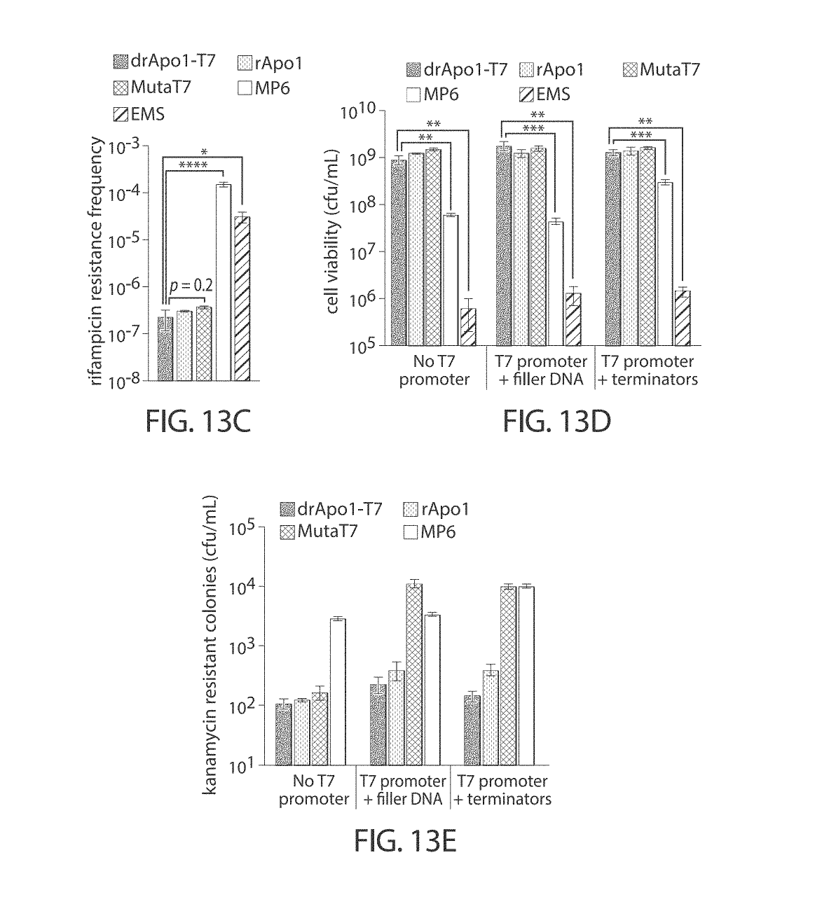

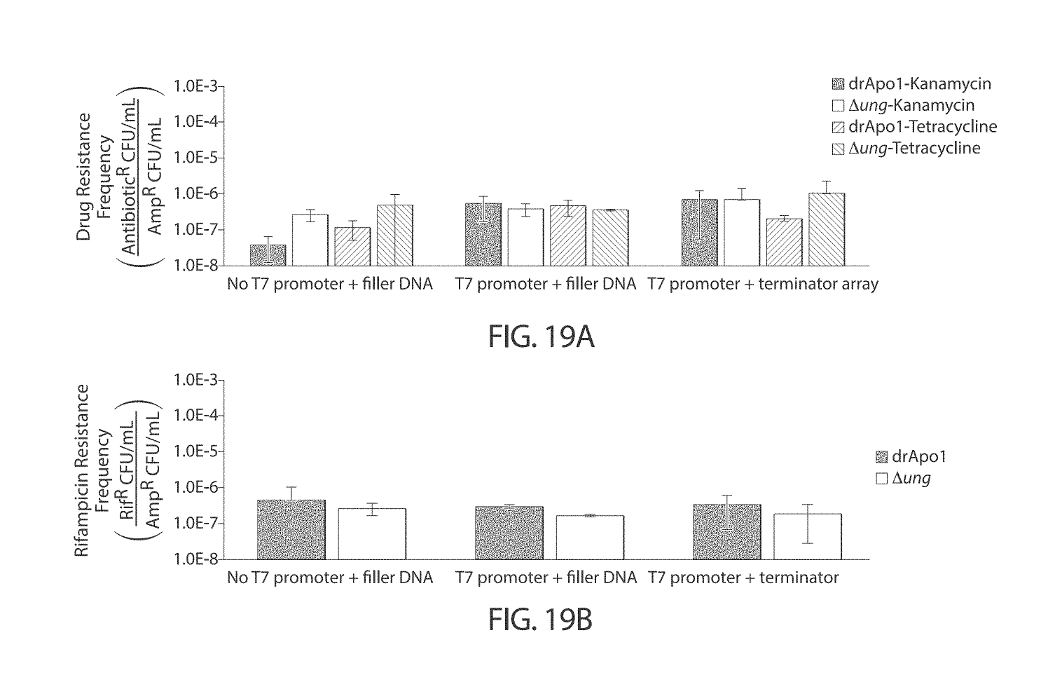

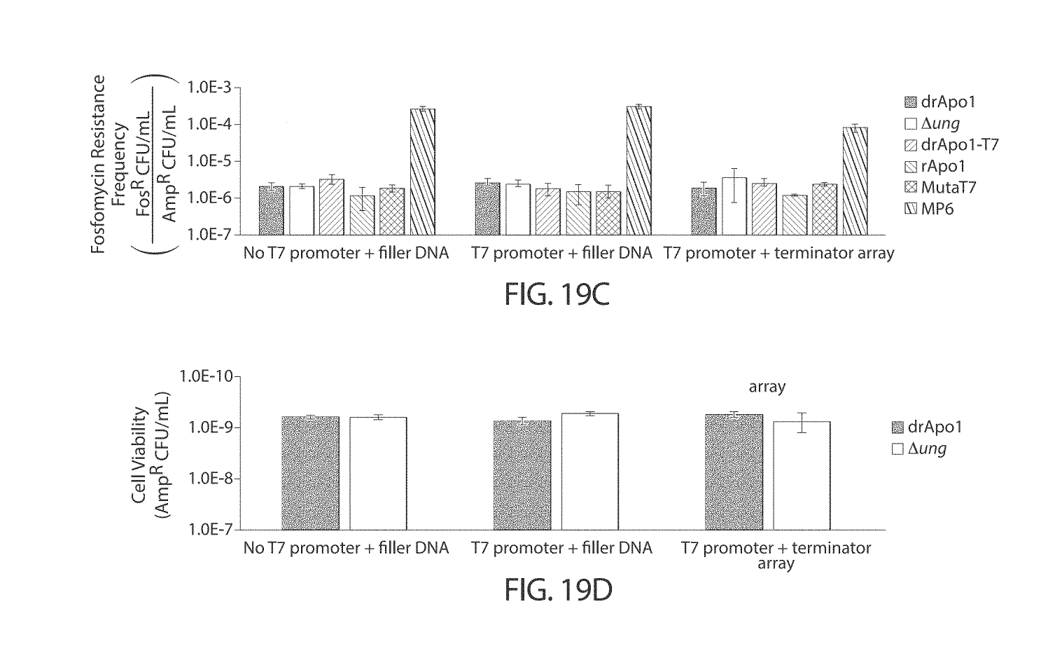

Mutation Assay: To assess mutagenesis rates, the control (Δung, rApo1, drApo1, and drApo1−T7; TABLE 8) and mutagenic strains (MutaT7 and MP6; TABLE 8) (Strep^{R}) carrying reporter plasmids (AmpR) were streaked on LB agar with 100 μg/mL streptomycin and 100 μg/mL ampicillin and grown at 37° C. for 24 h in order to obtain clones. Single colonies were picked in triplicate for each sample and used to inoculate 5 mL LB with 100 μg/mL streptomycin, 100 μg/mL ampicillin, and 25 mM arabinose (with 10 μg/mL chloramphenicol for the MP6 strain, TABLE 8), then shaken at 250 r.p.m. and 37° C. for 24 h to accumulate mutations during growth. 1 mL aliquots of each culture were pelleted at 6000×g for 3 min and resuspended in 1 mL LB to remove arabinose. Each resuspension was plated on LB agar plates with 50 μg/mL tetrazolium chloride (a metabolic contrast dye for visualizing colonies) and the antibiotics indicated below to analyze mutation rates and viability:
50 μL of a 100,000-fold dilution of each resuspension was plated on LB agar with 100 μg/mL streptomycin, 100 μg/mL ampicillin, and 50 μg/mL tetrazolium chloride. For samples from the MP6 strain, owing to lower growth of that strain, 50 μL of a 10,000-fold dilution of each resuspension was plated to obtain a more accurate count. The colony counts from these plates were used to calculate the cell viability (i.e., the number of live, ampicillin resistant cells) in CFU/mL for each sample (FIG. 13D).
50 μL of each resuspension was plated on LB agar plates with 200 μg/mL kanamycin and 50 μg/mL tetrazolium chloride. The colony counts from these plates were used to calculate the number of kanamycin resistant mutants in CFU/mL for each sample (FIG. 13E). The number of kanamycin resistant mutants in CFU/mL was divided by the number of live ampicillin resistant cells in CFU/mL for each sample to obtain the kanamycin resistant mutation frequency (FIG. 13B).
50 μL of each resuspension was plated on LB agar plates with 20 μg/mL tetracycline and 50 μg/mL tetrazolium chloride. The colony counts from these plates were used to calculate the number of tetracycline resistant mutants in CFU/mL for each sample. The number of tetracycline resistant mutants in CFU/mL was divided by the number of live ampicillin resistant cells in CFU/mL for each sample to obtain the tetracycline resistant mutation frequency (FIG. 13B).
50 μL of each resuspension was plated on LB agar plates with 100 μg/mL rifampicin and 50 μg/mL tetrazolium chloride. The colony counts from these plates were used to calculate the number of rifampicin resistant mutants in CFU/mL for each sample. The number of rifampicin resistant mutants in CFU/mL was divided by the number of live ampicillin resistant cells in CFU/mL for each sample to obtain the rifampicin resistant mutation frequency (FIG. 13C).
50 μL of each resuspension was plated on LB agar plates with 100 μg/mL fosfomycin and 50 μg/mL tetrazolium chloride. The colony counts from these plates were used to calculate the number of fosfomycin resistant mutants in CFU/mL for each sample. The number of fosfomycin resistant mutants in CFU/mL was divided by the number of live ampicillin resistant cells in CFU/mL for each sample to obtain the rifampicin resistant mutation frequency (FIG. 19C).

Plates were incubated at 37° C. for 48 h, then imaged by inverting the plates onto transparencies and scanning on a document scanner at a resolution of 400 dots per inch (d.p.i.). The colonies were then counted using the software OpenCFU (3.9.0) (Geissmann, PLoS One. 2013; 8(2): e5), with the minimum colony radius set to 3, the maximum colony radius set to 50, and the regular threshold set to 4.

The same assay as above was also used to assess the mutation rate of the ugi rApo1, ugi MutaT7, and ugi drApo1−T7 strains (TABLE 8), except that instead of arabinose either 0 mM or 1 mM isopropyl β-D-1-thiogalactopyranoside (IPTG) was added to the liquid overnight cultures as a control or to induce mutagenesis, respectively.

TABLE 8
Strain table. The genotypes of strains used in this work
are shown. The “x<>y” notation indicates
a replacement of “x” with “y” through lambda red recombineering.

| Strain | Genotype |
|---|---|
| DH10B | F⁻ araD139 Δ(araA-leu)7697 ΔlacX74 galE15 galK16 galU |
| (Durfee et | hsdR2 relA1 rpsL150(Str^{R}) spoT1 φp80lacZΔM15 endA1 |
| al., J. | nupG recA1 e14⁻ mcrA Δ(mrr hsdRMS-mcrBC) |
| Bacteriol. | |
| 2008 April; | |
| 190(7): | |
| 2597-606) | |
| Δung | DH10B Δung ΔmotAB ΔcsgABCDEFG |
| rApo1 | DH10B Δung ΔmotAB ΔcsgABCDEFG [P_{lacI}<>P_{tac}] |
| | Δ(araA-leu)7697::[P_{A1lacO-Tenth} rApo1] |
| drApo1 | DH10B Δung ΔmotAB ΔcsgABCDEFG [P_{lacI}<>P_{tac}] |
| | Δ(araA-leu)7697::[P_{A1lacO-Tenth} drApo1] |
| MutaT7 | DH10B Δung ΔmotAB ΔcsgABCDEFG [P_{lacI}<>P_{tac}] |
| | Δ(araA-leu)7697::[P_{A1lacO-Tenth} MutaT7] |
| drApo1 − T7 | DH10B Δung ΔmotAB ΔcsgABCDEFG [P_{lacI}<>P_{tac}] |
| | Δ(araA-leu)7697::[P_{A1lacO-Tenth} drApo1 − T7] |
| MP6 | DH10B Δung ΔmotAB ΔcsgABCDEFG MP6(Cm^{R}) |
| MutaT7- | DH10B Δung [P_{lacI}<>P_{tac}] Δ(araA-leu)7697::[P_{A1lacO-Tenth} |
| csg⁺ mot⁺ | MutaT7] |
| ung+ | DH10B ung⁺ ΔmotAB ΔcsgABCDEFG |
| drApo1 − T7 | DH10B ung⁺ ΔmotAB ΔcsgABCDEFG [P_{lacI}<>P_{tac}] |
| ung⁺ | Δ(araA-leu)7697::[P_{A1lacO-Tenth} drApo1 − T7] |
| drApo1 + T7 | DH10B ung⁺ ΔmotAB ΔcsgABCDEFG [P_{lacI}<>P_{tac}] |
| ung⁺ | Δ(araA-leu)7697::[P_{A1lacO-Tenth} drApo1 T7] |
| (Note: | |
| drApo1 | |
| and T7 | |
| expressed | |
| as separate | |
| proteins) | |
| ugi rApo1 | DH10B ung⁺ ΔmotAB ΔcsgABCDEFG [P_{lacI}<>P_{tac}] |
| | Δ(araA-leu)7697::[P_{A1lacO-Tenth} ugi rApo1] |
| ugi | DH10B ung⁺ ΔmotAB ΔcsgABCDEFG [P_{lacI}<>P_{tac}] |
| MutaT7 | Δ(araA-leu)7697::[P_{A1lacO-Tenth} ugi MutaT7] |
| ugi | DH10B ung⁺ ΔmotAB ΔcsgABCDEFG [P_{lacI}<>P_{tac}] |
| drApo1 − T7 | Δ(araA-leu)7697::[P_{A1lacO-Tenth} ugi drApo1 − T7] |

Chemical Mutagenesis with ethyl methanesulfonate (EMS): Mutagenesis with EMS was performed as previously described (Cupples and Miller, Proc. Natl. Acad. Sci. U.S.A. 1989 July; 86(14): 5345-49). An overnight culture of each sample was subcultured and grown until it reached a density of 2-3×10⁸ cells per mL (log phase). 5 mL aliquots of cells were chilled on ice, washed twice with sodium phosphate buffer (pH=7), and resuspended in 1 mL of 1×PBS in a 1.5 mL Eppendorf tube. EMS was added while cold by pipetting 14 μL of EMS into 1 ml of resuspended cells. Eppendorfs were sealed and mixed at 1000 r.p.m. for 60 min at 37° C. The cells were then washed twice with LB and resuspended in 1 mL of LB. Immediately after washing, a viability measurement was performed by plating 50 μL of a 10,000-fold dilution of each culture on LB agar with 100 μg/mL streptomycin, 100 μg/mL ampicillin, and 50 μg/mL tetrazolium chloride. After 48 h of incubation, plates were imaged on a document scanner as described above. The number of live ampicillin resistant colonies were counted after EMS treatment in CFU/mL to measure the viability after mutagen treatment (FIG. 13D). For mutation rate assessment, 500 μL of the post-EMS-treated resuspension was inoculated into 5 ml of LB with 100 μg/mL streptomycin and 100 μg/mL ampicillin. The cultures were grown at 37° C. for 20 h, then 50 μL of each culture was plated on LB agar with 50 μg/mL tetrazolium chloride and 100 μg/mL rifampicin. 50 μL of a 100,000-fold dilution of each culture was also plated on LB agar with 100 μg/mL streptomycin, 100 μg/mL ampicillin, and 50 μg/mL tetrazolium chloride. After 48 h of incubation, plates were imaged on a document scanner as described above. The number of rifampicin resistant mutants in CFU/mL was divided by the number of live ampicillin resistant cells in CFU/mL for each sample to obtain the rifampicin resistant mutation frequency (FIG. 13D).

Continuous Culturing and Sequencing of the Dual T7 Promoter Reporter Plasmid: The dual T7 promoter reporter plasmid was continuously cultured in the MutaT7-csg⁺ mot⁺ strain (TABLE 8) in a 70 mL culture in a round-bottomed flask that was slowly stirred in a 37° C. mineral oil bath. The culture was aerated through a needle that was connected to a standard aquarium pump and LB with 100 μg/mL streptomycin, 100 μg/mL ampicillin, and 0.5% isopropanol (as an antifoaming agent) was fed into the culture via a needle connected to a peristaltic pump at a rate of ˜0.5 volumes/h. Fractions were collected every 3 d for 12 d. Each fraction was plated for single colonies on LB agar with 100 μg/mL ampicillin and 10 clones from each fraction were Sanger-sequenced by colony PCR with the primers 1493 and 1494 (TABLE 10).

Continuous Culturing and Sequencing of the T7 Promoter+Filler DNA and T7 Promoter+Terminators Reporter Plasmids Reporter Plasmids: The T7 promoter+filler DNA and T7 promoter+terminators reporter plasmids were continuously cultured in the Δung (negative control), MutaT7, and MP6 strains (TABLE 8) in 20 mL cultures using a previously described multiplex bioreactor setup (Miller et al., J. Vis. Exp. 2013 Feb. 23; (72): e50262). The reactor was stored in a 37° C. warm room and was aerated and stirred with aquarium pumps. LB with 100 μg/mL streptomycin, 100 μg/mL ampicillin, 100 μg/mL cycloheximide, 0.01% (v/v) antifoam-204, and 150 μg/mL arabinose (+10 μg/mL chloramphenicol in the case of the MP6 strain (TABLE 8)) was pumped into each reaction vessel at a rate of 0.87 volumes/h. Fractions were collected every 3 d. Each fraction was plated on LB agar with 100 μg/mL streptomycin and 100 μg/mL ampicillin and 12 single colonies from each plate were grown in 5 mL LB with 100 μg/mL ampicillin. DNA was isolated from each overnight culture using the Qiaprep 96 Turbo Miniprep Kit and quantified using the PicoGreen assay.

Library Construction and Next Generation Sequencing: Libraries were prepared using a miniaturized version of Nextera XT. Briefly, 0.5 ng of input DNA was subjected to a 1/12 scale reaction of Illumina Nextera XT performed on a TTP Labtech Mosquito HV using combinatorial dual indexing (V_{final}=4 μl). Completed libraries were size selected using SPRI beads at 0.7× volume and pooled before sequencing on an Illumina MiSeq using 150 nt paired end reads (v2 chemistry). Sequencing reads were aligned against respective plasmid sequences using bwa mem (v. 0.7.12-r1039) (Li, arXiv preprint arXiv. 16 Mar. 2013; 1303.3997), with flag-t 16, and sorted and indexed bam files were generated using samtools (v 1.3) (Li et al., Bioinformatics. 2009 Aug. 15; 25(16): 2078-79). These bam files were processed using samtools mpileup with flags-excl-flags 2052, -d 10000000 and the same plasmid reference sequences used for mapping (Li et al., Bioinformatics. 2011 Nov. 1; 27(21): 2987-93). Read coverages and alleles counts and frequencies were tabulated at each position of the reference sequence in each sample for down-stream analysis. Only positions with greater than 10-fold coverage in all replicates of each sample were included in the analysis. Fixed variant alleles (present at greater than 85% frequency) for each sample are reported. Sanger sequencing was also performed on a PCR amplicon from 96 clones of Δung (negative control) and MutaT7 strains (TABLE 8) after 15 d of continuous culture carrying the T7 promoter+terminators reporter plasmid. The primers 2165 and 1197 (TABLE 10) were used to amplify and Sanger sequence the Kan^{R} gene.

Lambda Red Recombineering: The E. coli genome was edited using seamless lambda red recombineering with ccdB counterselection, as previously described (Wang et al., Nucleic Acids Res. 2014 March; 42(5): e37). Cells were transformed with the temperature-sensitive psc101-gbaA recombineering plasmid, plated on LB agar with 10 μg/mL tetracycline, and incubated for 24 h at 30° C. Colonies were selected and grown in LB containing 10 μg/mL tetracycline overnight at 30° C. (18-21 h). Overnight cultures were diluted 25-fold in LB with 10 μg/mL tetracycline and grown at 30° C. for ˜2 h until attaining an OD₆₀₀ of 0.3-0.4. The ccdA antitoxin and recombineering machinery were then induced by adding arabinose and rhamnose to a final concentration of 2 mg/mL each and then growing the cultures at 37° C. for 40 min to an OD₆₀₀ of ˜0.6. The cultures were then placed on ice, washed twice with ice-cold sterile ddH₂O, resuspended in ˜25 μL of ice-cold sterile ddH₂O, and electroporated with ˜200 ng of the appropriate kan-ccdB targeting cassette (1.8 kV, 5.8 ms, 0.1 cm cuvette, BioRad Micropulser). The cells were then recovered in super optimal broth with catabolite repression (SOC) with 2 mg/mL arabinose at 30° C. for 2 h, then plated on LB agar plates with 50 μg/mL kanamycin and 2 mg/mL arabinose and incubated for 24 h at 30° C. Colonies that grew under these conditions had incorporated the kan-ccdB targeting cassette and were picked and grown in LB with 50 μg/mL kanamycin and 2 mg/mL arabinose at 30° C. for 18-21 h. The cultures were then diluted 25-fold in LB with 50 μg/mL kanamycin and 2 mg/mL arabinose and grown at 30° C. for ˜2 h until they reached an OD₆₀₀ of 0.3-0.4. The recombineering machinery was then induced by adding rhamnose to a final concentration of 2 mg/mL and then growing the cultures at 37° C. for 40 min to an OD₆₀₀ of ˜0.6. The cultures were then placed on ice, washed twice with ice-cold sterile ddH₂O, resuspended in ˜25 μL of ice-cold sterile ddH₂O, and electroporated with ˜200 ng of the final targeting cassette intended to replace the kan-ccdB cassette currently integrated in the genome (1.8 kV, 5.8 ms, 0.1 cm cuvette, Bio-Rad Micropulser). The cells were then recovered in SOC with 2 mg/mL arabinose at 30 C for 2 h, and then were washed once with LB to remove the arabinose and prevent continued production of the ccdA antitoxin. The cultures were then plated on LB agar plates at various dilutions with 100 μg/mL streptomycin and incubated for 24 h at 37° C. Without the ccdA antitoxin, the ccdB toxin will kill cells that have not replaced the integrated kan-ccdB cassette with the final targeting cassette. The colonies that grow should have the final targeting cassette integrated, but were screened by PCR or sequencing to confirm cassette integration as some colonies may simply inactive the ccdB toxin. Once a clone with the desired change was found, the temperature-sensitive psc101-gbaA recombineering plasmid was cured by plating on LB agar with 100 μg/mL streptomycin, incubating at 42° C. for 18-21 h, streaking a colony from the plate on LB agar with 100 μg/mL streptomycin, and incubating at 42° C. for another 18-21 h. The colonies from the second plate were grown in LB with 100 μg/mL streptomycin at 37° C. to generate glycerol stocks. The colonies were also incubated in LB with 10 μg/mL tetracycline at 30° C. to ensure tetracycline sensitivity and confirm that the recombineering plasmid was successfully cured. The various strains used in this work (TABLE 8) were generated using the primers in TABLE 10.

TABLE 9
List of strain modifications.

| | | KanccdB cassette | Final targeting | |
|---|---|---|---|---|
| | | primers used with | cassette oligos or | |
| | | R6K-kan-ccdB | primers and template | |
| Modification | Genotype | template plasmid | (if applicable) | Purpose of modification |
| Insertion of | Δ(araA- | dAraLeu7697 | dAraLeu7697-rApoI | rApo1 and MutaT7 were |
| rApo1 or | leu)7697f::[BBa_J23114 | kanccdB F and | and dAraLeu7697-T7 | inserted into the DH10B |
| MutaT7 | rApo1 or | dAraFeu7697 | used | genome between base pairs |
| | MutaT7] | kanccdB R | to amplify from | 62,378 and 62,379 at the |
| | | | BBa_J23114 lacO | seam of the large Δ(araA- |
| | | | rApo1 or BBa_J23114 | leu)7697 deletion (Durfee et |
| | | | lacO MutaT7 | al., J. Bacteriol. 2008 April; |
| | | | | 190(7): 2597-606). |
| ung Deletion | Δung | 5′-Ung | oligos delUng S and | To minimize dU→dC repair, |
| | | kanccdB and | delUng AS (annealed | uracil DNA glycosylase (ung) |
| | | 3′-Ung | oligos) | was deleted (Duncan, J. |
| | | kanccdB | | Bacteriol. 1985 November; 164(2): |
| | | | | 689-95). |
| Increasing | [P_{lacI}<>P_{tac}] | 5′- | pLacI::pTac S and | In an attempt to get very low |
| lacI | | pLacI::kanccdB | pLacI::pTac AS | basal expression from lacI |
| expression | | and 3′- | (annealed oligos) | repressed promoters, the |
| | | pLacI::kanccdB | | endogenous P_{lacI} promoter |
| | | | | was replaced with the strong |
| | | | | P_{tac} promoter (Glascock et al., |
| | | | | Gene. 1998 Nov. 26; 223(1- |
| | | | | 2): 221-31). |
| Replacement | Δ(araA- | 5′- | PA1lacO-1 F and | The BBa_J23114 promoter |
| of promoter | leu)7697::[P_{A1lacO-Tenth} rApo1 | prApoI::kanccdB | PA1lacO-1 R used to | from the Anderson Collection |
| BBa_J23114 | or MutaT7] | and 3′- | amplify from pA1lacO- | (Anderson, J. C. Anderson |
| with P_{A1lacO-Tenth} | | prApoI::kanccdB | tenth gene block | promoter collection. |
| | | | (TABLE 10) | Available: |
| | | | | parts.igem.org/Promoters/Catalog/Anderson) |
| | | | | that controlled the expression of |
| | | | | rApo1 or MutaT7 from the |
| | | | | DH10B genome was replaced |
| | | | | with the tightly-repressed |
| | | | | promoter P_{A1lacO-Tenth}, which is |
| | | | | a weaker version of the |
| | | | | P_{A1lacO} promoter (Camsund et |
| | | | | al., J. Biol. Eng. 2014 Jan. 27; |
| | | | | 8(1): 4). |
| Deactivation | Δ(araA- | 5′- | drApoI S and drApoI | The E63Q mutant of rApo1 |
| of rApoI | leu)7697::[P_{A1lacO-Tenth} | drApoI::kanccdB | AS (annealed oligos) | cytidine deaminase has been |
| | drApo1 or | and 3′- | | shown to be catalytically |
| | drApo1 − T7] | drApoI::kanccdB | | inactive (Navaratnam et al., |
| | | | | Cell. 1995 Apr. 21; 81(2): |
| | | | | 187-95). E63Q mutant |
| | | | | negative control strains were |
| | | | | made. |
| Reinsertion | ung⁺ | 5′-Ung | Ung-Forward and Ung- | Used to reverse the ung |
| of ung | | kanccdB and | Reverse used to amplify | deletion and generate ung+ |
| | | 3′-Ung | from DH10B genomic | control strains. |
| | | kanccdB | DNA | |
| Insertion of | ugi | 5′-ugi kanccdB | Ugi-Forward and Ugi- | UGI is a small protein that |
| ugi | | and 3′-ugi | Reverse used to amplify | inhibits uracil DNA |
| | | kanccdB | from MP6 | glycosylase (UNG) (Badran |
| | | | | and Liu, Nat. Commun. 2015 |
| | | | | Oct. 7; 6: 8425). UGI was |
| | | | | inserted under the P_{A1lacO-Tenth} |
| | | | | promoter preceding MutaT7 |
| | | | | as an alternate strategy for |
| | | | | minimizing dU→dC repair. |

Deleting the motAB and csgABCDEFG operons through DIRex lambda red recombineering to decrease biofilm formation in bioreactor experiments: Deletions of the motAB operon (Pratt and Kolter, Mol. Microbiol. 1998 October; 30(2): 285-93) and the csgABCDEFG (Prigent-Combaret et al., Environ. Microbiol. 2000 August; 2(4): 450-64) have been shown to produce strains of E. coli that are deficient in biofilm formation. To minimize inlet line contamination and clogs in bioreactor experiments owing to biofilms, the motAB and csgABCDEFG operons were deleted using one-step DIRex lambda red recombineering (Näsvall, PLoS One. 2017 Aug. 30; 12(8): e0184126). The motAB targeting half-cassettes were amplified from R6K-AmilCP-kan-ccdB using the primers delmotDF and AmilCP-KanR and from R6K-kan-ccdB-AmilCP using the primers del-motDR and KanF-AmilCP (TABLE 10). The motAB half cassettes were co-electroporated to replace motAB with a kan-ccdB cassette flanked by large AmilCP inverted repeats nested between short 30 bp direct repeats. The repeat architecture leads to a high rate of spontaneous excision that was selected for using ccdB counterselection to obtain a markerless deletion of motAB. This procedure was then repeated to delete the csgABCDEFG operon. The csgABCDEFG targeting half-cassettes were amplified from R6K-AmilCP-kan-ccdB using the primers delcsgDF and AmilCP-KanR and from R6K-kan-ccdB-AmilCP using the primers delcsgDR and KanF-AmilCP (TABLE 10).

TABLE 10
Primer and oligo table. Primers used for lambda red recombineering,
restriction cloning of the terminator arrays, colony PCR, and
Sanger sequencing.

| Primer Name | SEQ ID NO | SEQUENCE |
|---|---|---|
| 5′-Ung | 29 | GCAGTTAAGCTAGGCGGATTGAAGATTCGCAGGAGAGCGAGATGGCT |
| kanccdB | | AACCCCTCATCAGTGCCAACATAGTAAG |
| 3′-Ung | 30 | AGCCGGGTGGCAACTCTGCCATCCGGCATTTCCCCGCAAATTTACTCAC |
| kanccdB | | TCCGCTCATTAGGCGGGC |
| delUng S | 31 | GCAGTTAAGCTAGGCGGATTGAAGATTCGCAGGAGAGCGAGATGGCT |
| | | AACAGTGAGTAAATTTGCGGGGAAATGCCGGATGGCAGAGTTGCCACC |
| | | CGGCT |
| delUng AS | 32 | AGCCGGGTGGCAACTCTGCCATCCGGCATTTCCCCGCAAATTTACTCAC |
| | | TGTTAGCCATCTCGCTCTCCTGCGAATCTTCAATCCGCCTAGCTTAACT |
| | | GC |
| 5′- | 33 | CGTTACTGGTTTCACATTCACCACCCTGAATTGACTCTCTTCCGGGCGC |
| pLacI: : kanccdB | | TCCCTCATCAGTGCCAACATAGTAAG |
| 3′- | 34 | TGGTGGCCGGAAGGCGAAGCGGCATGCATTTACGTTGACACCATCGAA |
| pLacI: : kanccdB | | TGCCGCTCATTAGGCGGGC |
| pLacI: : pTac | 35 | ATTCACCACCCTGAATTGACTCTCTTCCGGGCGCTCATTATACGAGCCG |
| AS | | ATGATTAATTGTCAACATTCGATGGTGTCAACGTAAATGCATGCCGCTT |
| | | C |
| pLacI: : pTac | 36 | GAAGCGGCATGCATTTACGTTGACACCATCGAATGTTGACAATTAATC |
| S | | ATCGGCTCGTATAATGAGCGCCCGGAAGAGAGTCAATTCAGGGTGGTG |
| | | AAT |
| delcsgDF | 37 | TTTCGCTTAAACAGTAAAATGCCGGATGATAATTCCGGCTTTTTTATCT |
| | | GTTTGTTGAAATATCGGAATTAAAAAAGAATTCAAAAAAAAGCCCGC |
| delcsgDR | 38 | GCAGCAGACCATTCTCTCCAGATTCATCTTATGCTCGATATTTCAACAA |
| | | ACAGATAAAAAAGCCGGAATTAAAAAAGAATTCAAAAAAAAGCCCGC |
| delmotDF | 39 | CTTCATCAAAAAATGTCTGATAAAAATCGCTTATATCCATGCTCACGCT |
| | | GGACATCATCCTTCCAGAATTAAAAAAGAATTCAAAAAAAAGCCCGC |
| delmotDR | 40 | CGCCTGACGACTGAACATCCTGTCATGGTCAACAGTGGAAGGATGATG |
| | | TCCAGCGTGAGCATGGAGAATTAAAAAAGAATTCAAAAAAAAGCCCG |
| | | C |
| KanF- | 41 | GCTCGACGTTGTCACTGAAGC |
| AmilCP | | |
| AmilCP- | 42 | CGCCGTCGGGCATGC |
| KanR | | |
| 5′- | 43 | GTCGTCACTCTATCTGGCGTCACACCTCTCAGAACACCAACAAACACG |
| drApoI: : kanccdB | | TTCCGCTCATTAGGCGGGC |
| 3′- | 44 | TTCGGGCAGAAGTAACGTTCGGTGGTGAATTTTTCGATGAAGTTAACTT |
| drApoI: : kanccdB | | CCCTCATCAGTGCCAACATAGTAAG |
| drApoI S | 45 | GTCGTCACTCTATCTGGCGTCACACCTCTCAGAACACCAACAAACACG |
| | | TTCAAGTTAACTTCATCGAAAAATTCACCACCGAACGTTACTTCTGCCC |
| | | GAA |
| drApoI AS | 46 | TTCGGGCAGAAGTAACGTTCGGTGGTGAATTTTTCGATGAAGTTAACTT |
| | | GAACGTGTTTGTTGGTGTTCTGAGAGGTGTGACGCCAGATAGAGTGAC |
| | | GAC |
| dAraLeu7697 | 47 | CAGCAGCAGAACGCCCGGCACGTGCTCTGCCAGTTGTTCAATGGCCTG |
| kanccdB F | | ATCCCTCATCAGTGCCAACATAGTAAG |
| dAraLeu7697 | 48 | TGAGCAGGCAATCAGCAGTTGATAACCCCGTTGCCGCGCCTGGCGTTC |
| kanccdB R | | AACCGCTCATTAGGCGGGC |
| dAraLeu7697- | 49 | CAGCAGCAGAACGCCCGGCACGTGCTCTGCCAGTTGTTCAATGGCCTG |
| rApoI | | ATTCGAGTTTATGGCTAGCTCAGTCC |
| dAraLeu7697- | 50 | TGAGCAGGCAATCAGCAGTTGATAACCCCGTTGCCGCGCCTGGCGTTC |
| T7 | | AACTGAAAATCTTCTCTCATCCGCC |
| 5′- | 51 | GCAGAACGCCCGGCACGTGCTCTGCCAGTTGTTCAATGGCCTGATTCG |
| prApoI: : kanccdB | | AGCCGCTCATTAGGCGGGC |
| 3′- | 52 | CGACGACGCAGGGTCGGGTCAACCGCAACCGGACCGGTTTCAGAAGA |
| prApoI: : kanccdB | | CATCCCTCATCAGTGCCAACATAGTAAG |
| PA1lacO-1 F | 53 | GCAGAACGCCCGGCAC |
| PA1lacO-1 R | 54 | CGACGCAGGGTCGGG |
| pA1lacO1- | 55 | GCAGAACGCCCGGCACGTGCTCTGCCAGTTGTTCAATGGCCTGATTCG |
| HA Tenth | | AGAAAGAGTGTTTATTTGTGAGCGGATAACAATTACAATTAGATTCAA |
| gene block | | TTGTGAGCGGATAACAATTTCACACAGGCTAGCGAATTCGAGCTCCCT |
| | | CTAGAAATAATTTTGTTTAACTTTAAGAAGGAGATATACCATGGGCAG |
| | | CAGCTACCCATACGACGTACCAGATTACGCTATGTCTTCTGAAACCGG |
| | | TCCGGTTGCGGTTGACCCGACCCTGCGTCGTCG |
| NheI- | 56 | CTAGCCAGCTTGGGTCTCCCTAGGTCGAGCTCCGTCGACCTAGCATAA |
| UUCG- | | CCCCGCGGGGCCTCTTCGGGGGTCTCGCGGGGTTTTTTGCTGAAAGG |
| BamHI S | | |
| NheI- | 57 | GATCCCTTTCAGCAAAAAACCCCGCGAGACCCCCGAAGAGGCCCCGCG |
| UUCG- | | GGGTTATGCTAGGTCGACGGAGCTCGACCTAGGGAGACCCAAGCTGG |
| BamHI AS | | |
| 1493 | 58 | AAAAAAAAGCTTGTTGACAATTAATCATCGGCATAGTATATCGGCATA |
| | | GTATAATACGACAAGGTGAGGAACTAAACCACGGGATCGGCCATTGA |
| | | AC |
| 1494 | 59 | AAAAAATTAATTAAGCTCTAGAGAATTGATCCCCTCAG |
| 2165 | 60 | TTGACAATTAATCATCGGCTCGAAGCTTG |
| 1197 | 61 | AAAAAAGGATCCTTCGCCATTCAGGCTGCG |
| 2062 | 71 | AAAGTGCCACCTGGCGG |
| Ung-Forward | 72 | GTGTATATATACTCCTCAAACACCCTTGAATCTTTG |
| Ung-Reverse | 73 | ATTGACGGTACGGGCAACG |
| 5′-ugi | 74 | ATATCTCCTTCTTAAAGTTAAACAAAATTATTTCTAGAGGGAGCTCGAA |
| kanccdB | | TCCCTCATCAGTGCCAACATAGTAAG |
| 3′-ugi | 75 | GATACTTAGATTCAATTGTGAGCGGATAACAATTTCACACAGGCTAGC |
| kanccdB | | GACCGCTCATTAGGCGGGC |
| Ugi-Forward | 76 | GATACTTAGATTCAATTGTGAGCGGATAACAATTTCACACAGGCTAGC |
| | | GATTAGGAGGAATTTCAACATGACAAATTTATCTGACATC |
| Ugi-Reverse | 77 | ATATCTCCTTCTTAAAGTTAAACAAAATTATTTCTAGAGGGAGCTCGAA |
| | | TCTTATAACATTTTAATTTTATTTTCTCCATTACTGTCTTG |
| rApo1StopD | 78 | GCCACTACCAGCGTCTGCCGCCGCACATCCTGTGGGCGACCGGTCTGA |
| F | | AATAAGGAGTGGCGGTAGCGGAATTAAAAAAGAATTCAAAAAAAAGC |
| | | CCGC |
| Apo1StopD | 79 | GTTCATATGGTATCCTCTTGAGCTCCCACTCCCTCCGCTACCGCCACTC |
| R | | CTTATTTCAGACCGGTCGCGAATTAAAAAAGAATTCAAAAAAAAGCCC |
| | | GC |

Separation of rApo1−T7 fusion (rApo1+T7) through DIRex lambda red recombineering: In order to generate a non-fusion control strain in which rApoI (or drApoI) and T7 are expressed separately from the same operon under the PA1_{lacO-Tenth} promoter, one-step DIRex lambda red recombineering was used to insert a stop codon at the end of the rApo1 gene. The rApo1Stop targeting half-cassettes were amplified from R6K-AmilCP-kan-ccdB using the primers rApo1StopDF and AmilCP-KanR and from R6K-kan-ccdB-AmilCP using the primers rApo1StopDR and KanF-AmilCP (TABLE 10). The rApo1Stop half cassettes were co-electroporated to insert a stop codon after rApo1 followed by a kan-ccdB cassette flanked by large AmilCP inverted repeats nested between short 30 bp direct repeats. Excision of the AmilCP-kan-ccdB-AmilCP cassette was selected for using ccdB counterselection to obtain a markerless insertion of a stop codon after rApo1.

Mutation Assay and Sequencing with the T7 Promoter+rpsL Reporter Plasmid: To assess the locations and types of mutations observed, the drApo1−T7 negative control strain and MutaT7 and MP6 mutagenic strains (TABLE 8) (Strep^{R}) carrying the T7 promoter+rpsL reporter plasmid (Amp^{R}) were streaked on LB agar with 100 μg/mL ampicillin and grown at 37° C. for 24 h in order to obtain clones. Single colonies were picked in triplicate for each sample and used to inoculate 5 mL LB with 100 μg/mL ampicillin and 25 mM arabinose (with 10 μg/mL chloramphenicol for the MP6 strain, TABLE 8), then shaken at 250 r.p.m. and 37° C. for 24 h to accumulate mutations during growth. 1 mL aliquots of each culture were pelleted at 6000×g for 3 min and resuspended in 1 mL LB to remove arabinose. 50 μL of a 100-fold dilution of each resuspension was plated on LB Lennox agar plates (pH 8.0) with 500 μg/mL streptomycin, 100 μg/mL ampicillin, and 50 μg/mL tetrazolium chloride. 48 colonies from each plate were picked for colony PCR using the primers 2062 and 1197 (TABLE 10). The amplicons were Sanger-sequenced using the primer 1197 (TABLE 10).

LacZα Activity Assay for Quantifying T7 and MutaT7 Processivity: In order to determine if the fusion of rApo1 to the N-terminus of T7 RNA polymerase affected the processivity and/or activity of the T7 RNA polymerase, the expression of the lacZα fragment from T7 promoters of varying upstream distances was measured via the cleavage of o-Nitrophenyl-β-galactoside (oNPG) using an assay adapted from a previous publication (Schaefer et al., Anal. Biochem. 2016 Mar. 29; 503: 56-57). LacZα reporter plasmids C1A through C1F (ChlorR) were transformed into the ung⁺, drApo1−T7 ung⁺ and drApo1+T7 ung⁺ strains (TABLE 8) and plated on LB agar with 25 μg/mL chloramphenicol and grown at 37° C. for 24 h in order to obtain clones. Colonies of each reporter/strain combination were picked in triplicate and grown in 200 μL LB with 25 μg/mL chloramphenicol and 1 mM IPTG in a parafilm-wrapped 96-well plate that was shaken at 220 r.p.m. at 30° C. for 22 h. IPTG was added to induce the expression of the lacZα fragment from the genome that complements the lacZα fragment, and to increase the expression of drApo1−T7 and T7 from the P_{AllacO}-Tenth promoter. 80 μL of each overnight culture was mixed with 120 μL Bgal mix (60 mM sodium dibasic, 40 mM sodium phosphate monobasic, 10 mM potassium chloride,1 mM magnesium sulfate, 26 mM 2-mercaptoethanol, 166 μg/mL egg-white lysozyme, 1.0 mg/mL oNPG, and 6.7% PopCulture lysis reagent) in a black, clear-bottomed 96-well plate. The OD₆₀₀ and OD₄₂₀ of each well was measured every 2 min over the course of 1 h in a Biotek Synergy H1 hybrid plate reader followed by double orbital shaking at 559 r.p.m. at 30° C. The oNPG cleavage activity of each well was calculated by measuring the slope of the linear region of each OD₄₂₀ trace, dividing by the initial OD₆₀₀ reading, and multiplying by 1000. The mean and standard deviation of each set of triplicates were calculated.

Episomal folA Directed Evolution Assay to Assess False Positive Frequency: To assess the effect that targeted versus global mutagenesis has on the false positive frequency of a directed evolution experiment, a model drug resistance evolution experiment was designed where the rate of true positive evolution corresponds to the frequency that drug resistance-conferring mutations appear in an episomal copy of a drug-sensitive gene. To create this system, the folA+T7 promoter plasmid (Amp^{R})—which contains the complete, endogenous folA promoter and coding sequence for dihydrofolate reductase followed by a T7 promoter pointing in the reverse direction—was transformed into MutaT7 and MP6 mutagenic strains (TABLE 8) (Strep^{R}). These strains were streaked on LB agar with 100 μg/mL ampicillin and grown at 37° C. for 24 h in order to obtain clones. Single colonies were picked in triplicate for each sample and used to inoculate 5 mL LB with 100 μg/mL ampicillin and 25 mM arabinose (with 10 μg/mL chloramphenicol for the MP6 strain (TABLE 8)), then shaken at 250 r.p.m. and 37° C. for 24 h to accumulate mutations during growth. 1 mL aliquots of each culture were pelleted at 6000×g for 3 min and resuspended in 1 mL LB to remove arabinose. 50 μL of a 100-fold dilution of each resuspension was plated on LB agar plates with 5 μg/mL trimethoprim (TMP) and 50 μg/mL tetrazolium chloride. 13-15 colonies from each plate were picked for colony PCR. Episomal folA was amplified and Sanger sequenced using the primers Alof-T7 S and 1197 (TABLE 10).

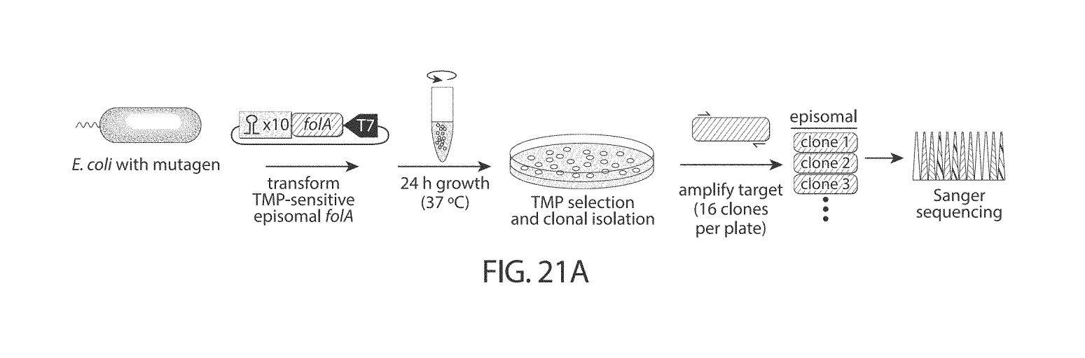

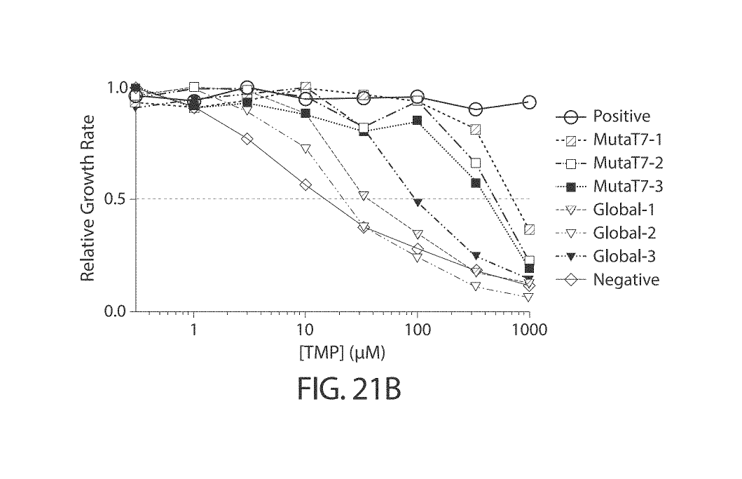

TABLE 11
Sanger sequencing data for FIGS. 21A-21B.

| | | Total | On-target | | Relative |
|---|---|---|---|---|---|
| Muta- | Biological | Colonies | folA Mutation | Off-target | On-target |
| gen | Replicate | Sequenced | (plasmid) | Mutation | Frequency |
| MutaT7 | 1 | 14 | 11 | 3 | 78.57% |
| | 2 | 15 | 8 | 7 | 53.33% |
| | 3 | 15 | 10 | 4 | 66.67% |
| | Total | 44 | 29 | 15 | 65.91% |
| MP6 | 1 | 15 | 0 | 15 | 0% |
| | 2 | 12 | 0 | 12 | 0% |
| | 3 | 16 | 0 | 16 | 0% |
| | Total | 43 | 0 | 43 | 0% |

Bacterial growth assay measuring trimethoprim drug resistance: Isolates were grown to stationary phase following overnight incubation at 37° C. in LB with 100 μg/mL ampicillin. Cultures were diluted 1:100 into a plate containing LB broth with increasing concentrations of TMP ranging from 1 μM to 1 mM. Growth of diluted samples was determined by measuring OD₆₀₀ every 5 min in a Biotek Synergy H1 hybrid plate reader followed by orbital shaking at 282 r.p.m. and incubation at 37° C. Maximal growth rate was determined by performing “Max V” calculation in Gen5 software, using a 5-point segment of each growth curve corresponding to the highest linear slope. Upon determining maximum growth rate within each sample, growth rates were normalized to the highest growth rate within each sample series yielding the relative growth rate at each TMP concentration (FIGS. 21A-21B).

### Example 9

### Design of Chimeric MutaT7 Protein

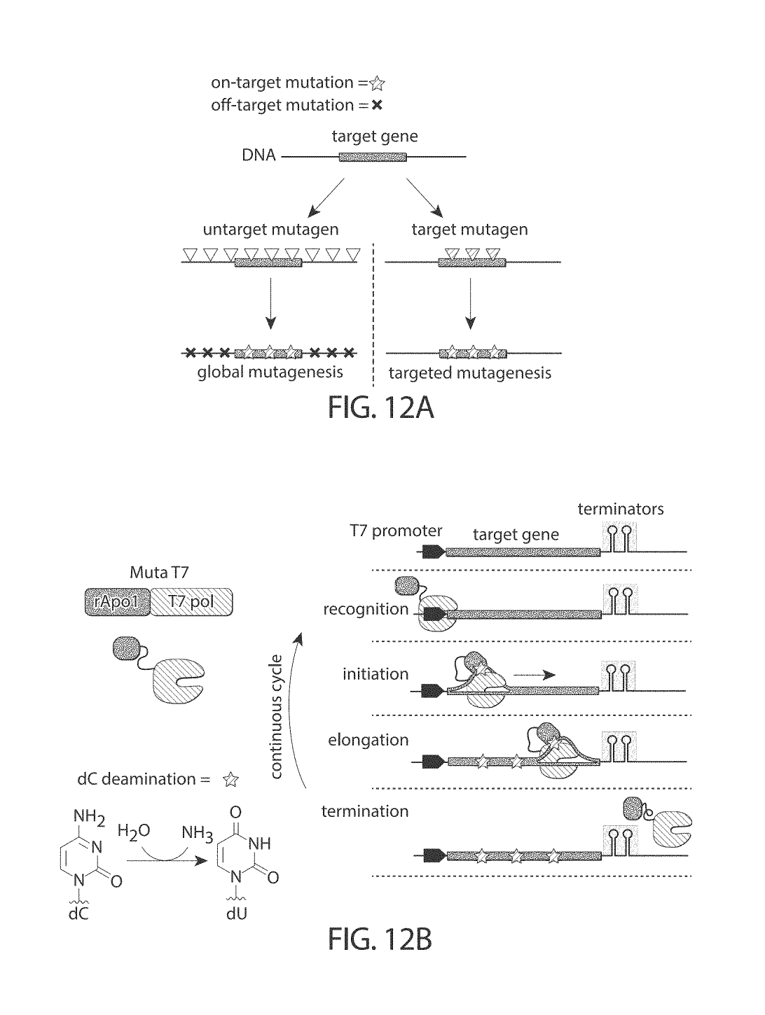

Traditional in vivo mutagenesis strategies rely on exogenous mutagens (e.g., high energy light or chemicals) (Cupples et al., Proc. Natl. Acad. Sci. U.S.A. 1989 July; 86(14): 5345-49; Tessman et al., 1965 Apr. 23; 148(3669): 507-8) or expressing mutagenic enzymes (e.g., XL1-Red (Greener et al., Mol. Biotechnol. 1997 April; 7(2): 189-95) or the MP6 plasmid (Badran A. H. and Liu D. R., Nat. Commun. 2015 Oct. 7; 6: 8425)). These global mutagenesis strategies can yield high mutation rates and diverse genetic landscapes. However, extensive mutations throughout the genome are problematic in many contexts, especially in directed evolution experiments (FIG. 12A). Off-target mutations outside the intended DNA region are often toxic when they occur in essential portions of the genome (Gerdes et al., J. Bacteriol. 2003 October; 185(19): 5673-84; Wang et al., Science. 2015 Nov. 27; 350(6264): 1096-101), a problem that limits library size and engenders rapid silencing of mutagenic plasmids. Global mutagens also introduce “parasite” variants into DNA libraries, originating from mutations outside the gene of interest that allow an organism to circumvent selection schemes (Tizei et al., Biochem. Soc. Trans. 2016 Aug. 15; 44(4): 1165-1175).

Targeted in vivo mutagenesis strategies have the potential to overcome these deficiencies. DNA-damaging enzymes fused to deactivated Cas9 nucleases can edit bases at specific genetic loci (Komor et al., Nature. 2016 May 19; 533(7603): 420-24; Nishida et al., Science. 2016 Sep. 16; 353(6305): pii: aaf8729; Komor et al., Sci. Adv. 2017 Aug. 30; 3(8): eaao4774; Gaudelli et al., Nature. 2017 Nov. 23; 551(7681): 464-71; Kim et al., Nat. Biotechnol. 2017 Apr. 10; 35(5): 475-480), but require many gRNAs to tile mutagenic enzymes throughout a target DNA that may be multi-kb in length (Hess et al., Nat. Methods. 2016 December; 13(12): 1036-42; Ma et al., Nat. Methods. 2016 December; 13(12): 1029-35). Moreover, the guide RNAs must be redesigned after each evolution round introduces new mutations in the target DNA. Another example is the use of an error-prone poll variant to selectively mutagenize genes on ColE1 plasmids, although this method is limited to Escherichia coli and can target mutations within only a few kb of the ColE1 origin (Camps et al., Proc. Natl. Acad. Sci. U.S.A. 2003 Aug. 8; 100(17): 9727-9732; Allen et al., Nucleic Acids Res. 2011 May 26; 39(16): 7020-7033). Error-prone replication mediated by the Ty1 retrotransposon specifically in yeast can also selectively mutate <5 kb genetic cargoes inserted into the retrotransposon (Crook et al., Nat. Commun. 2016 Oct. 17; 7: 13051). Other targeted mutation methods in yeast include oligo-mediated genome engineering (DiCarlo et al., ACS Synth. Biol. 2013 Dec. 20; 2(12): 741-749), which can be labor-intensive, and an orthogonal replication system (Ravikumar et al., Nat. Chem. Biol. 2014 Feb. 2; 10(3): 175-177), which was developed specifically in yeast.

It was hypothesized herein that a processive, DNA-traversing biomolecule tethered to a DNA-damaging enzyme could provide a generalizable solution to the problem of targeting mutations across large, yet still well-defined, DNA regions. Monomeric RNA polymerases possess inherently high promoter specificity (Rong et al., J. Biol. Chem. 1998 Apr. 24; 273(17): 10253-60) and processivity (Thiel et al., J. Gen. Virol. 2001 June; 82(6): 1273-81). Cytidine deaminases are potent DNA-damaging enzymes that can act on single-stranded DNA substrates during transcription (Ramiro et al., Nat. Immunol. 2003 May; 4(5): 452-56). We envisioned that a chimeric “MutaT7” protein consisting of a cytidine deaminase (rApo1) fused to T7 RNA polymerase (T7-pol) would, therefore, allow us to target mutations specifically to any DNA region lying downstream of a T7 promoter (FIG. 12B), provided the T7 promoter is not present elsewhere in the genome.

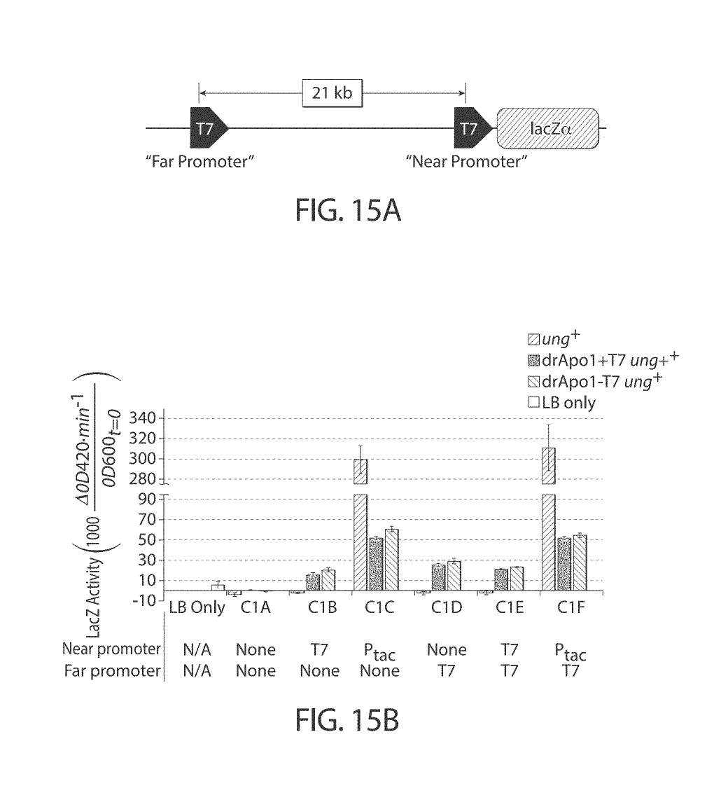

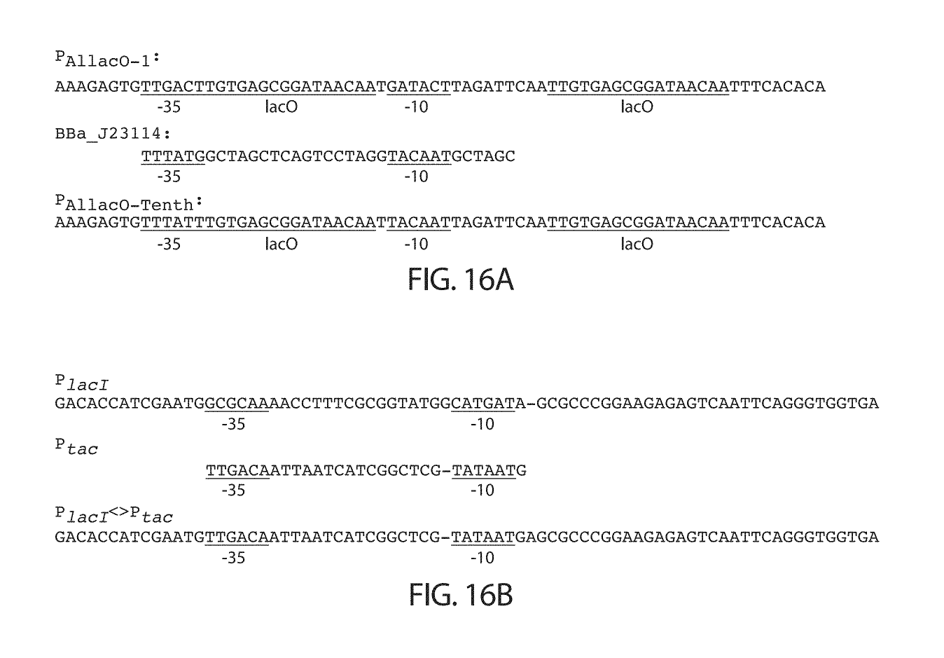

To begin, a lacZ expression assay (Schaefer et al., Anal. Biochem. 2016 Mar. 29; 503: 56-57) was used to show that T7-Pol tolerated an rApo1 N-terminal fusion and still efficiently transcribed tens of kilobases (FIGS. 15A-15B). Next, the MutaT7 gene was integrated under control of a weak promoter into the genome of E. coli lacking uracil N-glycosylase (Δung) (FIGS. 16A-16B and TABLE 8). Deleting ung inhibits repair of deoxyuridine to deoxycytidine and increases mutagenesis rates (Nilsen et al., Mol. Cell. 2000 June; 5(6): 1059-1065; Alsøe et al., Sci. Rep. 2017 Aug. 3; 7(1): 719), especially in the context of cytidine deaminases (Petersen-Mahrt et al., Nature. 2002 Jul. 4; 418(6893): 99-103).

### Example 10

### Characterization of Targeted Mutagenesis of MutaT7

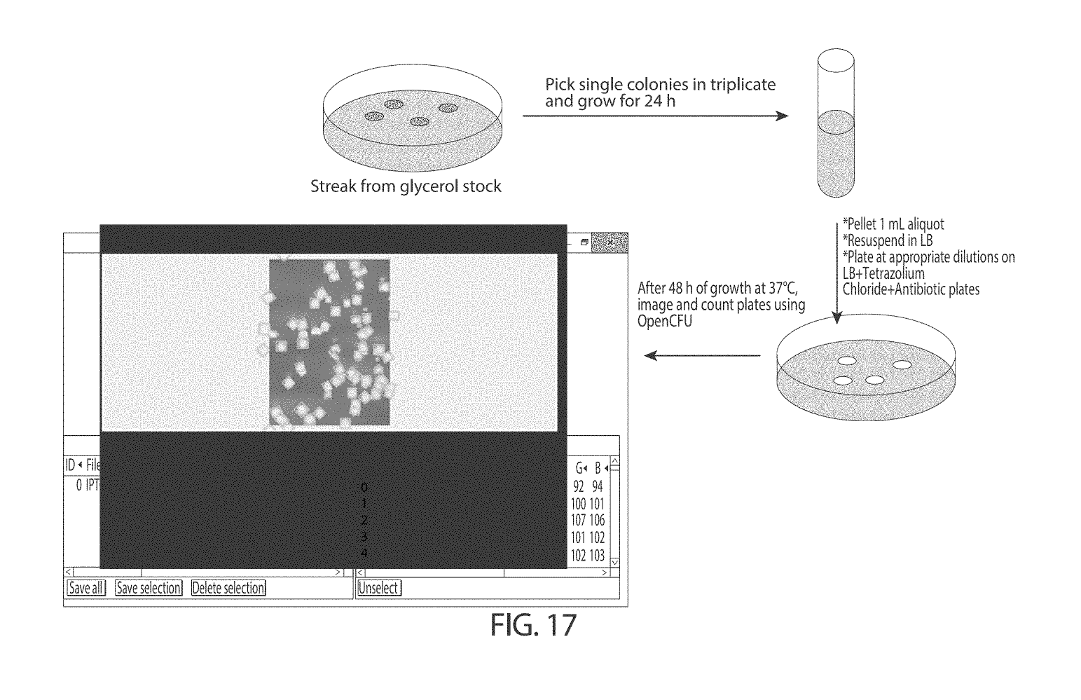

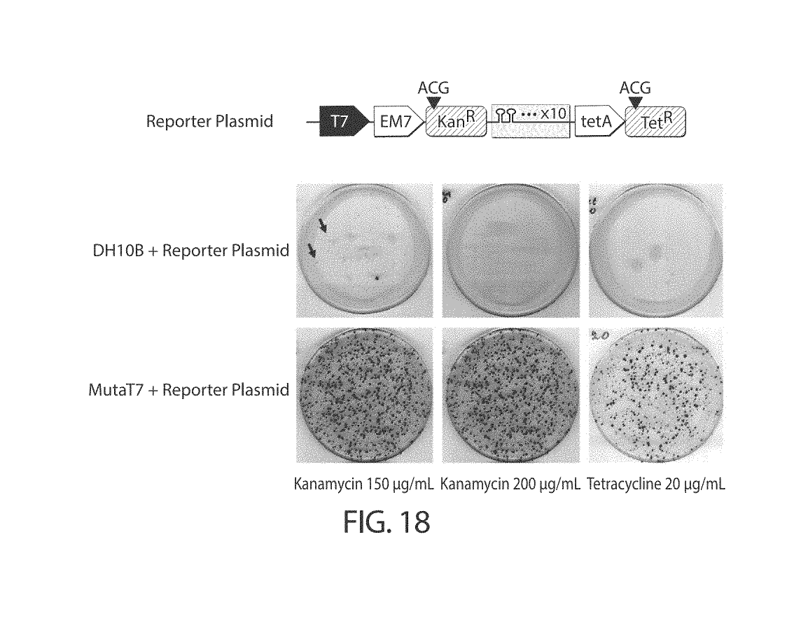

Targeted mutagenesis was assayed using a codon reversion assay based on reporter plasmids either having or lacking a T7 promoter sequence upstream of silent drug resistance genes with ACG triplets in place of ATG start codons (FIG. 13A, FIG. 17, FIG. 18, FIG. 19A). The kanamycin resistance gene (Kan^{R}) was placed immediately downstream of the T7 promoter. In this assay, successful C to T mutagenesis at the Kan^{R} start codon yields kanamycin-resistant colonies. Global mutagens such as the MP6 plasmid yielded high levels of kanamycin-resistant colonies regardless of the T7 promoter, consistent with a lack of promoter-based targeting (FIG. 13B). In contrast, MutaT7 strains attained significant kanamycin resistance only when reporter plasmids possessed a T7 promoter upstream of the Kan^{R} gene (FIG. 13B). Expression of a catalytically dead version of MutaT7 (drApo1−T7) (Navaratnam et al., Cell. 1995 Apr. 21; 81(2): 187-95) yielded kanamycin resistance frequencies similar to background levels, indicating that T7 activity alone was not responsible for the observed increase in kanamycin resistance (FIG. 13B).

T7 promoter-dependent Kan^{R} mutagenesis by MutaT7 shows that mutagenesis can be targeted to a desired DNA region near a T7 promoter. Because T7-pol is highly processive, it was anticipated that mutations would also be introduced further downstream of the T7 promoter. MutaT7 processivity was assayed by inserting a tetracycline-resistance (Tet^{R}) gene with an inactive, ACG start codon ˜1.6 kb downstream of the Kan^{R} gene (FIG. 13A). High levels of MutaT7-dependent tetracycline resistance were observed only in reporter strains having the T7 promoter, consistent with targeted and processive introduction of mutations across a lengthy, multi-kb DNA region (FIG. 13B). Global mutagens again generated tetracycline-resistant colonies at high frequency in all cases, irrespective of the T7 promoter (FIG. 13B).

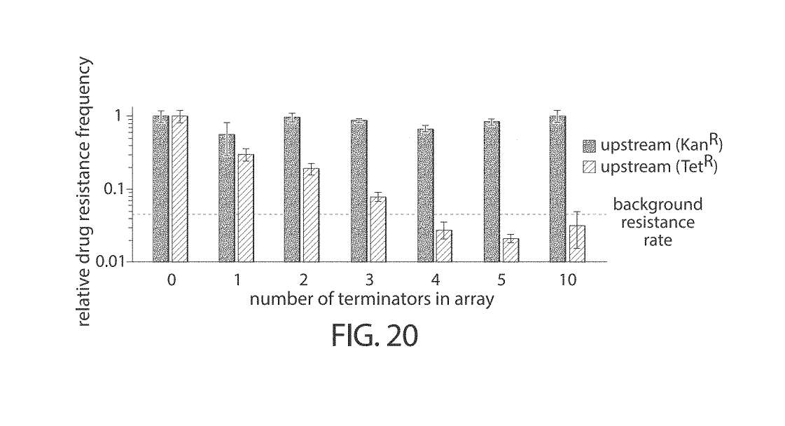

Targeted mutagenesis using the processive MutaT7 chimera requires not just recruitment to a DNA locus, but also termination at the end of targeted DNA. To address termination, Kan^{R}/Tet^{R} reporter plasmids were used in which the silent, start codon-defective resistance genes were separated by one or more T7 terminators (FIG. 13A). Upon assaying for drug resistance, it was found that four copies of the T7 terminator fully constrained mutagenesis to the intended upstream Kan^{R} gene (FIG. 20). Using this terminator array, tetracycline resistance was observed for MutaT7 strains similar to background levels, whereas kanamycin resistance remained high (FIG. 13B). Global mutagens again induced high levels of kanamycin- and tetracycline-resistance, irrespective of the terminator array (FIG. 13B).

To further assess whether MutaT7 induces mutagenesis specifically on the target DNA, the evolution of resistance to rifampicin (Garibyan et al., DNA Repair. 2003 May; 2(5): 593-8) and fosfomycin (Nilsson et al., Antimicrob. Agents Chemother. 2003 September; 47(9): 2850-58) was evaluated. Resistance can derive from diverse genomic mutations such that the appearance of resistant colonies correlates with off-target mutation rates in the genome (Badran and Liu, Nat. Commun. 2015 Oct. 7; 6: 8425; Garibyan et al., DNA Repair. 2003 May; 2(5): 593-8), analogous to cheating parasites in directed evolution schemes. Selection on either rifampicin- or fosfomycin-treated plates revealed that MutaT7-expressing samples displayed drug resistance frequencies comparable to background. In contrast, high frequencies of antibiotic resistance were observed in all global mutagenesis samples (FIG. 13C and FIGS. 19B-19C).

Additional experiments were directed at using MutaT7 to evolve ectopically expressed folA gene variants that confer trimethoprim resistance. The folA gene encodes dihydrofolate reductase, and folA mutations are just one of many potential routes to trimethoprim resistance (Acar and Goldstein, Rev. Infect. Dis. 1982 Mar.-Apr. 4; 4(2): 270-275). Either global mutagenesis or MutaT7 was used to mutagenize E. coli carrying a T7-targeted episomal copy of folA. Sanger-sequencing was then performed on colonies that grew on trimethoprim plates. 29 of 44 trimethoprim-resistant colonies mutagenized using MutaT7 had a mutation known to confer resistance (Herrington et al., J. Basic Microbiol. 2002; 42(3): 172) in the episomal folA promoter (TABLE 9, FIGS. 21A-21B). In contrast, none of the 43 trimethoprim-resistant colonies obtained using the global mutagen contained mutations in the episomal folA gene. Instead, they presumably gained trimethoprim resistance via undesired mutations in the E. coli genome. The ability of MutaT7 to generate a high rate of true positives in the desired episomal gene target, whereas global mutagenesis exclusively generated cheaters (false positives), highlights a key advantage of MutaT7.

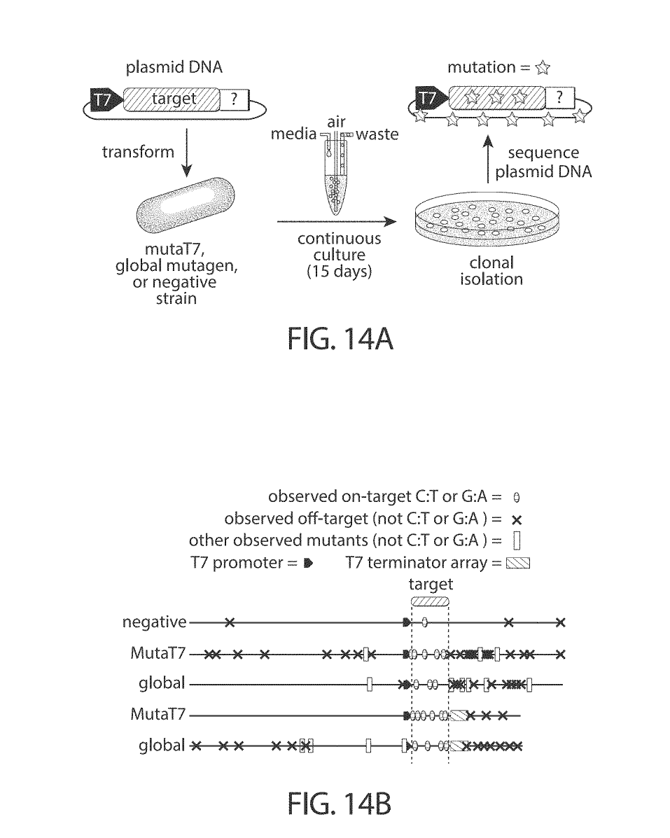

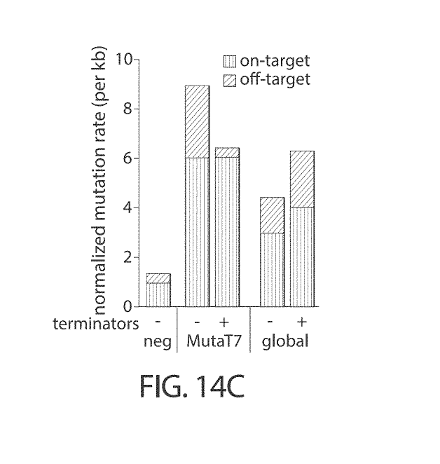

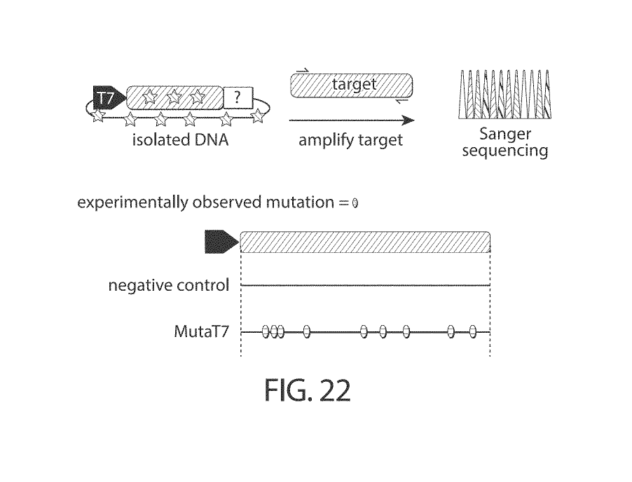

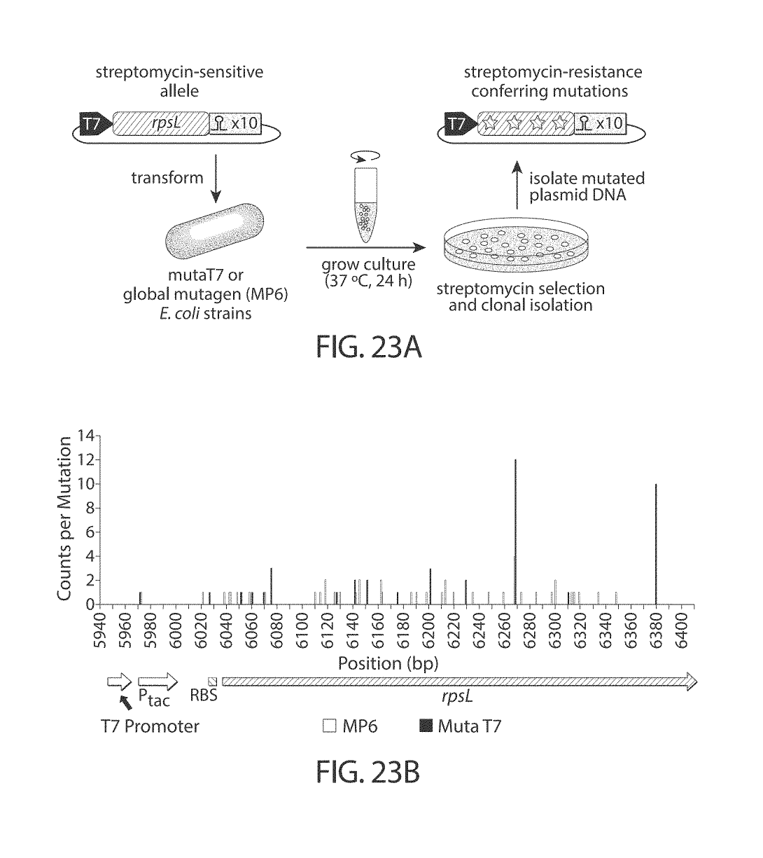

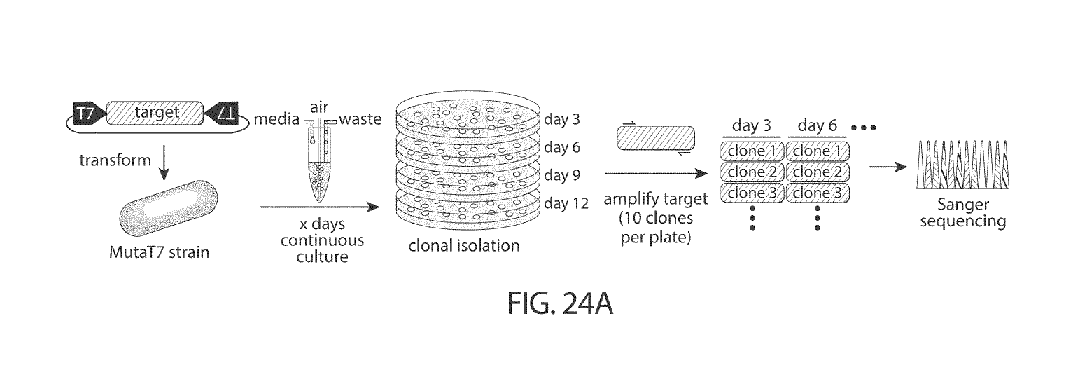

DNA sequencing was then used to better understand the processivity and targeting of MutaT7 mutagenesis. An E. coli population expressing MutaT7 and the episomal Kan^{R}/Tet^{R} reporter plasmid was allowed to drift in the absence of selection pressure for 15 days prior to isolation of episomal DNA from clones (FIG. 14A). Sanger sequencing of the target episomal region revealed mutations at multiple loci throughout the Kan^{R} target gene, independent of selection pressure (FIG. 22). In a separate experiment where the target DNA consisted of an episomal rpsL allele (initially sensitive to streptomycin) downstream of a T7 promoter (FIG. 23A), the processivity of MutaT7 was further evaluated. Sanger sequencing of streptomycin-resistant DNA isolated from a MutaT7-expressing strain of E. coli again revealed that multiple mutations appeared throughout the targeted rpsL gene, with ˜90% C to T mutations and ˜10% G to A mutations (FIG. 23B).

### Example 11

### Characterization of MutaT7 Toxicity

Another benefit of targeted mutagenesis is the capacity to attain much larger library sizes by avoiding toxic mutations in essential, off-target genes. On the basis of the apparently low off-target mutagenesis rate of MutaT7, it was hypothesized that E. coli carrying MutaT7 would have significantly higher viability than bacteria treated with global mutagens. Indeed, consistent with prior work (Badran and Liu, Nat. Commun. 2015 Oct. 7; 6: 8425), a very low viability was observed in all populations treated with global mutagens. In contrast, populations expressing MutaT7 possessed viability similar to untreated cells (FIG. 13D and FIG. 19D). The total number of kanamycin-resistant colonies was similar between MutaT7 and globally mutagenized samples (FIG. 13E) despite the somewhat lower mutagenesis rate of the MutaT7 construct relative to MP6 (FIG. 13B; the average kanamycin resistance frequency for MutaT7 was 6.7×10⁻⁶ versus 5.7×10⁻⁵ for MP6). This observation highlights that the use of MutaT7 to maximize on-target mutations while simultaneously minimizing off-target mutations results in larger productive library sizes.

### Example 12

### Characterization of MutaT7 On- and Off-Mutation Rates

Next, Illumina sequencing was used to identify mutations anywhere in the episomal reporter DNA sequence obtained from clones of the E. coli populations in FIG. 14A. This experiment assesses on- versus off-target mutagenesis across a ˜10 kb stretch of DNA containing only ˜1 kb of intended target DNA. MutaT7 samples displayed many mutations throughout the episome when the terminator array was removed but the T7 promoter was maintained (FIG. 14B). Treatment with the MP6 global mutagen also led to mutations throughout the entire episomal DNA. In contrast, mutations in MutaT7 strains appeared almost exclusively within the Kan^{R} target gene when both a promoter and terminator array were present, even after 15 days of continuous culturing (FIG. 14B). Upon normalizing on- and off-target mutation rates, it was observed that the few off-target mutations found on plasmids with a terminator from MutaT7 strains were present only to the same extent as in the control sample not treated with any mutagen (FIG. 14C).

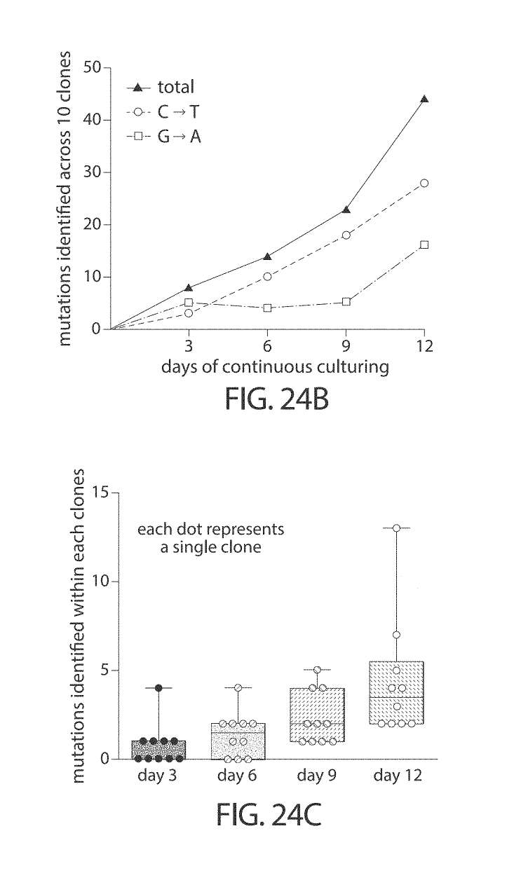

A disadvantage of MutaT7 is its limited mutational spectrum and an apparent strand bias observed in the sequencing results showing that C to T transitions were predominantly obtained in the sense strand using a single T7 promoter (FIG. 14C). It was hypothesized that the mutational spectrum could be doubled by introducing a second T7 promoter that would recruit MutaT7 to the 3′-end of the target DNA and enable processive activity in the opposing direction. Indeed, installing an additional antisense T7 promoter led to the accumulation of both G to A and C to T mutations throughout the target gene during continuous culturing (FIGS. 24A-24B). Furthermore, the average number and range of mutations per clone increased over time (FIG. 24C). The latter observation indicates that, in contrast to global mutagenesis methods where the organism often rapidly silences mutagen expression, the high on-target to off-target mutation ratio of MutaT7 enabled long-term maintenance of mutagen expression in cells.

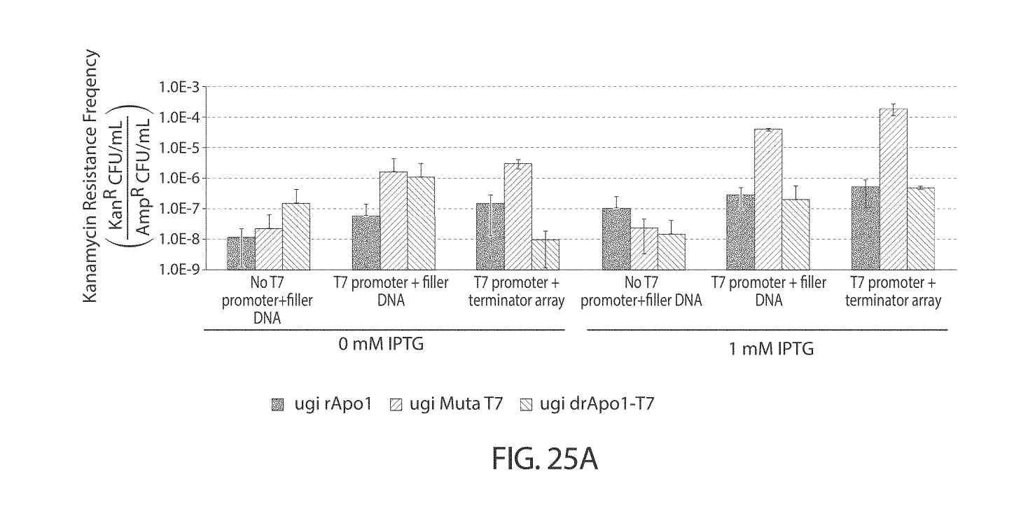

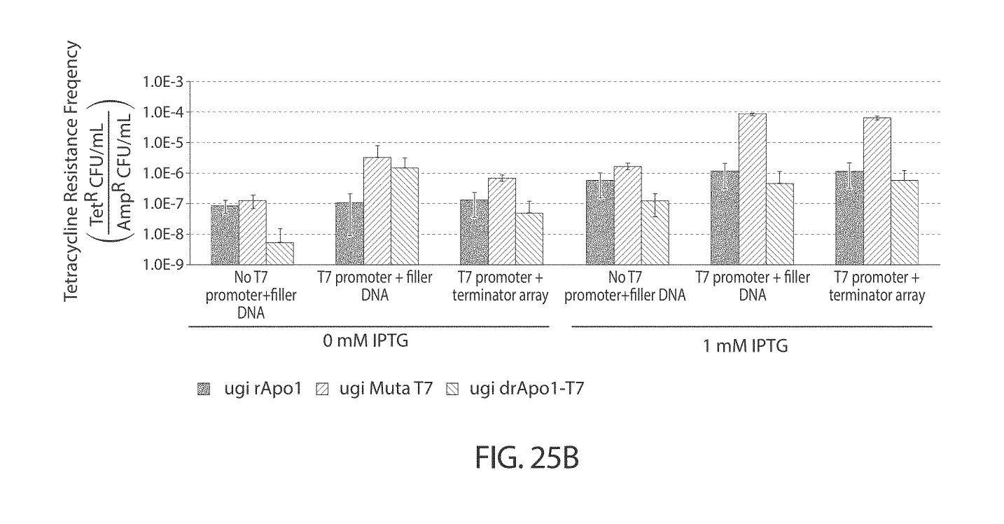

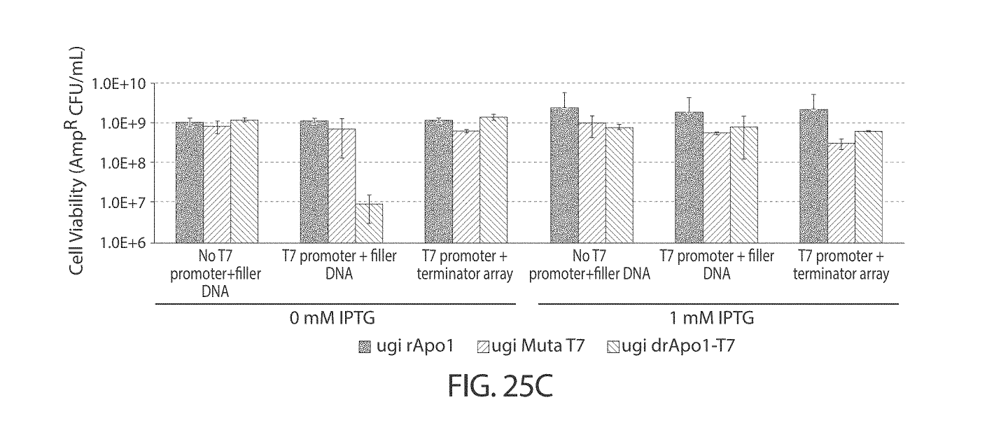

It was also observed that repair of deoxyuridine must be prevented to observe significant mutagenesis with MutaT7 (also observed with other cytidine deaminase-based systems (Badran and Liu, Nat. Commun. 2015 Oct. 7; 6: 8425; Komor et al., Nature. 2016 May 19; 533(7603): 420-24)). Although Δung cells were used to address this issue in the aforementioned experiments, a more flexible alternative is to co-express MutaT7 with the uracil glycosylase inhibitor (UGI; a protein that can inhibit UNG activity in many prokaryotes and eukaryotes (Badran and Liu, Nat. Commun. 2015 Oct. 7; 6: 8425; Komor et al., Nature. 2016 May 19; 533(7603): 420-24; Serrano-Heras et al., Nucleic Acids Res. 2007 Aug. 13; 35(16): 5393-5401)). Such co-expression resulted in a high rate of mutagenesis similar to that achieved using Δung cells (FIGS. 25A-25C). UGI thus eliminates the need to delete ung to achieve efficient mutagenesis with MutaT7, significantly increasing the flexibility of the system.

In summary, the processively-acting MutaT7 chimera can selectively direct mutations to large, yet well-defined, regions of DNA in vivo. Utilizing other base editing enzymes (Gaudelli et al., Nature. 2017 Nov. 23; 551(7681): 464-71) in concert with cytidine deaminase will significantly widen the mutational spectrum of MutaT7 and further enable the creation of rich and diverse DNA libraries in vivo. Moreover, DNA-modifiers fused to T7 could facilitate targeted epigenetic studies (DeNizio et al., Curr. Opin. Chem. Biol. 2018 Feb. 13; 45: 10-17). The ubiquitous applicability of T7-pol in diverse organisms (McBride et al., Proc. Natl. Acad. Sci. U.S.A. 1994 Jul. 19; 91(15): 7301-7305; Lieber et al., Eur. J. Biochem., 1998 Oct. 1; 217(1): 387-94; Weinstock et al., Nat. Methods. 2016 Aug. 29; 13(10): 849-851; Dower and Rosbash, RNA. 2002 May; 8(5): 686-697) suggests that MutaT7 will prove useful in a broad range of evolutionary and synthetic biology settings.

### Example 13

### Materials and Methods for Example 14

The following strains were constructed using lambda red recombineering as described in Example 8.

TABLE 12
Strain table. The genotypes of strains used in this work
are shown. The “x<>y” notation indicates
a replacement of “x” with “y” through lambda red recombineering.

| Strain | Genotype |
|---|---|
| DH10B | F⁻ araD139 Δ(araA-leu)7697 ΔlacX74 galE15 galK16 galU |
| (Durfee et | hsdR2 relA1 rpsL150(Str^{R}) spoT1 φp80lacZΔM15 endA1 |
| al., J. | nupG recA1 e14⁻ mcrA Δ(mrr hsdRMS-mcrBC) |
| Bacteriol. | |
| 2008 April; | |
| 190(7): | |
| 2597-606) | |
| tadA-Only | DH10B Δung ΔmotAB ΔcsgABCDEFG [P_{lacI}<>P_{tac}] |
| | Δ(araA-leu)7697::[P_{A1lacO-Tenth} tadA] |
| tadA- | DH10B Δung ΔmotAB ΔcsgABCDEFG [P_{lacI}<>P_{tac}] |
| XTEN-T7 | Δ(araA-leu)7697::[P_{A1lacO-Tenth} tadA-XTEN-T7] |
| tadA-GGS- | DH10B Δung ΔmotAB ΔcsgABCDEFG [P_{lacI}<>P_{tac}] |
| T7 | Δ(araA-leu)7697::[P_{A1lacO-Tenth} tadA-GGS-T7] |

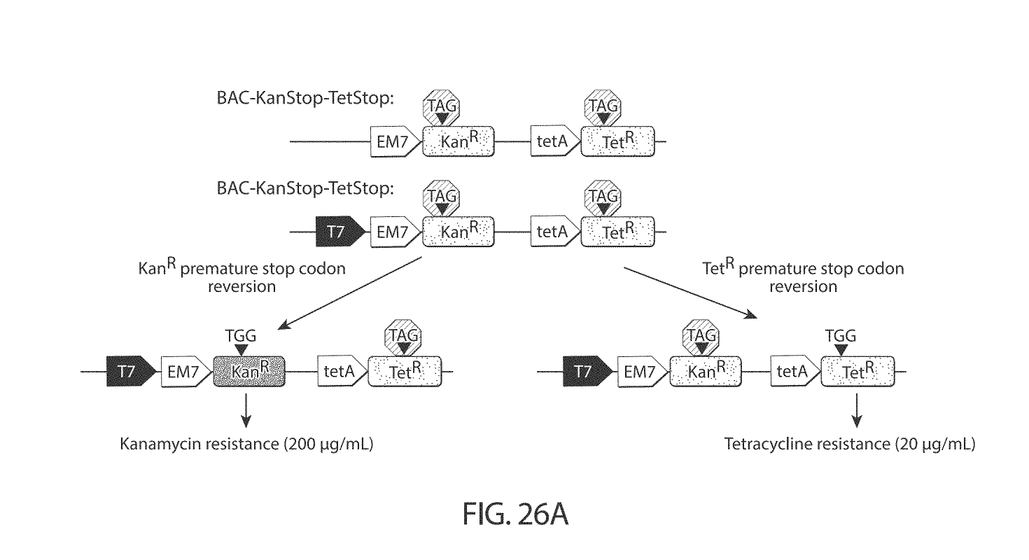

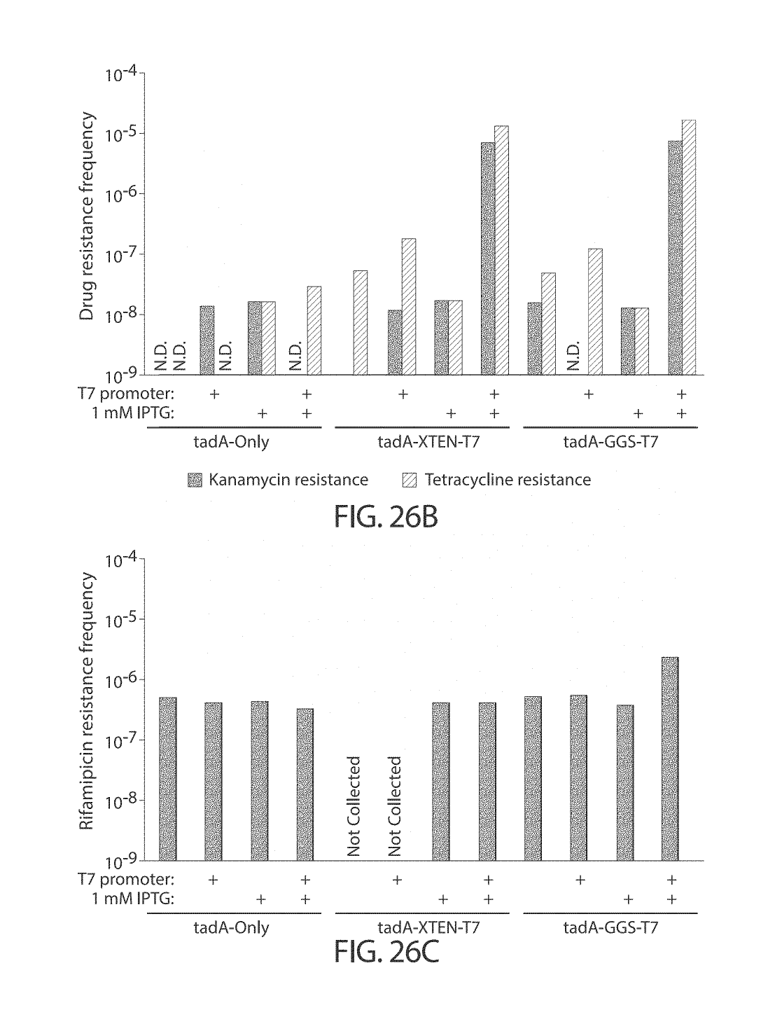

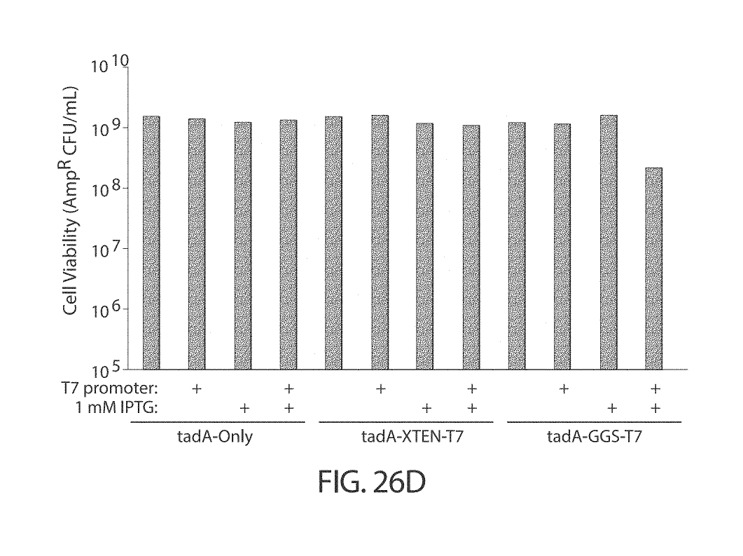

To assess mutagenesis rates, the control (tadA-Only) and mutagenic strains (tadA-XTEN-T7 and tadA-GGS-T7) (Strep^{R}) carrying reporter plasmids (BAC-KanStop-TetStop or BAC-T7-KanStop-TetStop, FIG. 26A) (Amp^{R}) were streaked on LB agar with 100 μg/mL streptomycin and 100 μg/mL ampicillin and grown at 37° C. for 24 h in order to obtain clones. Single colonies were picked for each sample and used to inoculate 5 mL LB with 100 μg/mL streptomycin,100 μg/mL ampicillin with or without 1 mM IPTG, then shaken at 250 r.p.m. and 37° C. for 24 h to accumulate mutations during growth. 1 mL aliquots of each culture were pelleted at 10000×g for 3 min and resuspended in 1 mL LB to remove IPTG. Each resuspension was plated on LB agar plates with 50 μg/mL tetrazolium chloride (a metabolic contrast dye for visualizing colonies) and the antibiotics indicated below to analyze mutations rates and viability:
50 μL of a 100,000-fold dilution of each resuspension was plated on LB agar with 100 μg/mL streptomycin, 100 μg/mL ampicillin, and 50 μg/mL tetrazolium chloride. The colony counts from these plates were used to calculate the cell viability (i.e., the number of live, ampicillin resistant cells) in CFU/mL for each sample (FIG. 26D).
50 μL of each resuspension was plated on LB agar plates with 200 μg/mL kanamycin and 50 μg/mL tetrazolium chloride. The colony counts from these plates were used to calculate the number of kanamycin resistant mutants in CFU/mL for each sample. The number of kanamycin resistant mutants in CFU/mL was divided by the number of live ampicillin resistant cells in CFU/mL for each sample to obtain the kanamycin resistant mutation frequency (FIG. 26B).
50 μL of each resuspension was plated on LB agar plates with 20 μg/mL tetracycline and 50 μg/mL tetrazolium chloride. The colony counts from these plates were used to calculate the number of tetracycline resistant mutants in CFU/mL for each sample. The number of tetracycline resistant mutants in CFU/mL was divided by the number of live ampicillin resistant cells in CFU/mL for each sample to obtain the tetracycline resistant mutation frequency (FIG. 26B).
50 μL of each resuspension was plated on LB agar plates with 100 μg/mL rifampicin and 50 μg/mL tetrazolium chloride. The colony counts from these plates were used to calculate the number of rifampicin resistant mutants in CFU/mL for each sample. The number of rifampicin resistant mutants in CFU/mL was divided by the number of live ampicillin resistant cells in CFU/mL for each sample to obtain the rifampicin resistant mutation frequency (FIG. 26C).

Plates were incubated at 37° C. for 48 h, then imaged by inverting the plates onto transparencies and scanning on a document scanner at a resolution of 400 dots per inch. The colonies were then counted using the software OpenCFU (3.9.0) (Geissmann, PLoS One. 2013; 8(2): e54072), with the minimum colony radius set to 3, the maximum colony radius set to 50, and the regular threshold set to 4.

### Example 14

### Characterization of TadA Fusion Proteins

To show that other types of mutations can be introduced using other DNA damaging agents fused to T7, a previously reported variant of tadA (Gaudelli et al., Nature. 2017 Nov. 23; 551(7681): 464-71, the entirety of which is incorporated herein by reference) was fused to T7 using two different linker sequences (GGS and XTEN) and placed under the control of an IPTG-inducible promoter (PAllacO-Tenth). This variant of tadA is able to make A to G mutations in DNA.

Mutagenesis assays were then carried out using these tadA-T7 E. coli strains and reporter plasmids that have defective resistance genes. For these assays, reporter plasmids were used that have defective kanamycin (Kan^{R}) and tetracycline (Tet^{R}) resistance genes (each having premature TAG stop codons). The BAC-KanStop-TetStop reporter plasmid lacks a T7 promoter, and should thus not be targeted by the tadA-XTEN-T7 or tadA-GGS-T7 fusion enzymes. The BAC-T7-KanStop-TetStop reporter plasmid has a T7 promoter preceding the defective KanR and TetR genes, which should allow tadA-XTEN-T7 or tadA-GGS-T7 fusion enzymes to mutate these genes, occasionally mutating the TAG stop codon to TGG and thus conferring antibiotic resistance (FIG. 26A).

Without the T7 promoter on the reporter plasmid, only a low level of resistance-conferring mutations were observed across all conditions, including with the tadA-Only control strain, which only expresses the tadA enzyme alone (FIG. 26B). A high level of mutagenesis was observed when the tadA-XTEN-T7 or tadA-GGS-T7 fusion enzymes were induced with 1 mM IPTG and when the reporter plasmid contained a T7 promoter, suggesting that the tadA-XTEN-T7 or tadA-GGS-T7 fusion enzymes specifically introduce A to G mutations downstream from a T7 promoter.

Furthermore, low levels of rifampicin resistance were observed across all conditions (FIG. 26C). The fact that very few rifampicin resistance-conferring mutations are occurring in the E. coli genome across all conditions suggests that the tadA-XTEN-T7 or tadA-GGS-T7 fusion enzymes have minimal off-target activity relative to the tadA-Only control. Finally, under most conditions, the expression of tadA-XTEN-T7 or tadA-GGS-T7 fusion enzymes did not negatively impact cell viability (FIG. 26D).

### Example 15

### Applications of Dynamic Targeted Hypermutation

DNA mutagenesis is an important and necessary step in all directed evolution methodologies, which are heavily utilized by academic and industrial labs around the world. Mutagenic technologies are particularly vital for research labs developing biomolecular drugs with novel actions or improved potency, as the identification of biomolecules with improved therapeutic properties inherently relies on some form of directed evolution. The recent implementation of biologics has further increased the demand for new and improved antibodies, vaccines, and recombinant proteins. As progress in biologic development is constrained by currently available methodologies for performing directed evolution, there is a widespread vested interest in more efficient and cost-effective mutagenic methods.

### Example 16

### Additional Sequences

*No T7 promoter reporter plasmid
(SEQ ID NO: 62)
ACAATTAATCATCGGCTCGAAGCTTGTTGACAATTAATCATCGGCATAGTATATCGGCATAGTATA
ATACGACAAGGTGAGGAACTAAACCACGGGATCGGCCATTGAACAAGATGGATTGCACGCAGGTT
CTCCGGCCGCTTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCGACCTGTCCG
GTGCCCTGAATGAACTGCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAAGTGCC
GGGGCAGGATCTCCTGTCATCTCACCTTGCTCCTGCCGAGAAAGTATCCATCATGGCTGATGCAAT
GCGGCGGCTGCATACGCTTGATCCGGCTACCTGCCCATTCGACCACCAAGCGAAACATCGCATCGA
GCGAGCACGTACTCGGATGGAAGCCGGTCTTGTCGATCAGGATGATCTGGACGAAGAGCATCAGG
GGCTCGCGCCAGCCGAACTGTTCGCCAGGCTCAAGGCGCGCATGCCCGACGGCGATGATCTCGTC
GTGACCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGATATTGCT
GAAGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGGGGATCAATTCTCTAGAGCTTAAT
TAACGCAGCCTGAATGGCGAATAGGGATCCTTGACAGCTTATCATCGATAAGCTTTAATGCGGTAG
TTTATCACAGTTGCTAACGCAGTCAGGCACCGTGTACGAATAGTTCGACAAAGATCGCATTGGTAA
TTACGTTACTCGATGCCATGGGGATTGGCCTTATCATGCCAGTCTTGCCAACGTTATTACGTGAATT
TATTGCTTCGGAAGATATCGCTAACCACTTTGGCGTATTGCTTGCACTTTATGCGTTAATGCAGGTT
ATCTTTGCTCCTTGGCTTGGAAAAATGTCTGACCGATTTGGTCGGCGCCCAGTGCTGTTGTTGTCAT
TAATAGGCGCATCGCTGGATTACTTATTGCTGGCTTTTTCAAGTGCGCTTTGGATGCTGTATTTAGG
CCGTTTGCTTTCAGGGATCACAGGAGCTACTGGGGCTGTCGCGGCATCGGTCATTGCCGATACCAC
CTCAGCTTCTCAACGCGTGAAGTGGTTCGGTTGGTTAGGGGCAAGTTTTGGGCTTGGTTTAATAGC
GGGGCCTATTATTGGTGGTTTTGCAGGAGAGATTTCACCGCATAGTCCCTTTTTTATCGCTGCGTTG
CTAAATATTGTCACTTTCCTTGTGGTTATGTTTTGGTTCCGTGAAACCAAAAATACACGTGATAATA
CAGATACCGAAGTAGGGGTTGAGACGCAATCGAATTCGGTATACATCACTTTATTTAAAACGATGC
CCATTTTGTTGATTATTTATTTTTCAGCGCAATTGATAGGCCAAATTCCCGCAACGGTGTGGGTGCT
ATTTACCGAAAATCGTTTTGGATGGAATAGCATGATGGTTGGCTTTTCATTAGCGGGTCTTGGTCTT
TTACACTCAGTATTCCAAGCCTTTGTGGCAGGAAGAATAGCCACTAAATGGGGCGAAAAAACGGC
AGTACTGCTCGGATTTATTGCAGATAGTAGTGCATTTGCCTTTTTAGCGTTTATATCTGAAGGTTGG
TTAGTTTTCCCTGTTTTAATTTTATTGGCTGGTGGTGGGATCGCTTTACCTGCATTACAGGGAGTGA
TGTCTATCCAAACAAAGAGTCATCAGCAAGGTGCTTTACAGGGATTATTGGTGAGCCTTACCAATG
CAACCGGTGTTATTGGCCCATTACTGTTTGCTGTTATTTATAATCATTCACTACCAATTTGGGATGG
CTGGATTTGGATTATTGGTTTAGCGTTTTACTGTATTATTATCCTGCTATCGATGACCTTCATGTTAA
CCCCTCAAGCTCAGGGGAGTAAACAGGAGACAAGTGCTTAGTGATCCAATTCTTGAAGACGAAAG
GGCCTCGTGATACGCCTATTTTTATAGGTTAATGTCATGATAATAATGGTTTCTTAGACGTCAGGTG
GCACTTTTCGGGGAAATGTGCCCGGTTAACGTGCCGGCACGGCCTGGGTAACCAGGTATTTTGTCC
ACATAACCGTGCGCAAAATGTTGTGGATAAGCAGGACACAGCAGCAATCCACAGCAGGCATACAA
CCGCACACCGAGGTTACTCCGTTCTACAGGTTACGACGACATGTCAATACTTGCCCTTGACAGGCA
TTGATGGAATCGTAGTCTCACGCTGATAGTCTGATCGACAATACAAGTGGGACCGTGGTCCCAGAC
CGATAATCAGACCGACAACACGAGTGGGATCGTGGTCCCAGACTAATAATCAGACCGACGATACG
AGTGGGACCGTGGTCCCAGACTAATAATCAGACCGACGATACGAGTGGGACCGTGGTTCCAGACT
AATAATCAGACCGACGATACGAGTGGGACCGTGGTCCCAGACTAATAATCAGACCGACGATACGA
GTGGGACCATGGTCCCAGACTAATAATCAGACCGACGATACGAGTGGGACCGTGGTCCCAGTCTG
ATTATCAGACCGACGATACGAGTGGGACCGTGGTCCCAGACTAATAATCAGACCGACGATACGAG
TGGGACCGTGGTCCCAGACTAATAATCAGACCGACGATACGAGTGGGACCGTGGTCCCAGTCTGA
TTATCAGACCGACGATACAAGTGGAACAGTGGGCCCAGAGAGAATATTCAGGCCAGTTATGCTTT
CTGGCCTGTAACAAAGGACATTAAGTAAAGACAGATAAACGTAGACTAAAACGTGGTCGCATCAG
GGTGCTGGCTTTTCAAGTTCCTTAAGAATGGCCTCAATTTTCTCTATACACTCAGTTGGAACACGAG
ACCTGTCCAGGTTAAGCACCATTTTATCGCCCTTATACAATACTGTCGCTCCAGGAGCAAACTGAT
GTCGTGAGCTTAAACTAGTTCTTGATGCAGATGACGTTTTAAGCACAGAAGTTAAAAGAGTGATAA
CTTCTTCAGCTTCAAATATCACCCCAGCTTTTTTCTGCTCATGAAGGTTAGATGCCTGCTGCTTAAG
TAATTCCTCTTTATCTGTAAAGGCTTTTTGAAGTGCATCACCTGACCGGGCAGATAGTTCACCGGG
GTGAGAAAAAAGAGCAACAACTGATTTAGGCAATTTGGCGGTGTTGATACAGCGGGTAATAATCT
TACGTGAAATATTTTCCGCATCAGCCAGCGCAGAAATATTTCCAGCAAATTCATTCTGCAATCGGC
TTGCATAACGCTGACCACGTTCATAAGCACTTGTTGGGCGATAATCGTTACCCAATCTGGATAATG
CAGCCATCTGCTCATCATCCAGCTCGCCAACCAGAACACGATAATCACTTTCGGTAAGTGCAGCAG
CTTTACGACGGCGACTCCCATCGGCAATTTCTATGACACCAGATACTCTTCGACCGAACGCCGGTG
TCTGTTGACCAGTCAGTAGAAAAGAAGGGATGAGATCATCCAGTGCGTCCTCAGTAAGCAGCTCC
TGGTCACGTTCATTACCTGACCATACCCGAGAGGTCTTCTCAACACTATCACCCCGGAGCACTTCA
AGAGTAAACTTCACATCCCGACCACATACAGGCAAAGTAATGGCATTACCGCGAGCCATTACTCCT
ACGCGCGCAATTAACGAATCCACCATCGGGGCAGCTGGTGTCGATAACGAAGTATCTTCAACCGG
TTGAGTATTGAGCGTATGTTTTGGAATAACAGGCGCACGCTTCATTATCTAATCTCCCAGCGTGGTT
TAATCAGACGATCGAAAATTTCATTGCAGACAGGTTCCCAAATAGAAAGAGCATTTCTCCAGGCA
CCAGTTGAAGAGCGTTGATCAATGGCCTGTTCAAAAACAGTTCTCATCCGGATCTGACCTTTACCA
ACTTCATCCGTTTCACGTACAACATTTTTTAGAACCATGCTTCCCCAGGCATCCCGAATTTGCTCCT
CCATCCACGGGGACTGAGAGCCATTACTATTGCTGTATTTGGTAAGCAAAATACGTACATCAGGCT
CGAACCCTTTAAGATCAACGTTCTTGAGCAGATCACGAAGCATATCGAAAAACTGCAGTGCGGAG
GTGTAGTCAAACAACTCAGCAGGCGTGGGAACAATCAGCACATCAGCAGCACATACGACATTAAT
CGTGCCGATACCCAGGTTAGGCGCGCTGTCAATAACTATGACATCATAGTCATGAGCAACAGTTTC
AATGGCCAGTCGGAGCATCAGGTGTGGATCGGTGGGCAGTTTACCTTCATCAAATTTGCCCATTAA
CTCAGTTTCAATACGGTGCAGAGCCAGACAGGAAGGAATAATGTCAAGCCCCGGCCAGCAAGTGG
GCTTTATTGCATAAGTGACATCGTCCTTTTCCCCAAGATAGAAAGGCAGGAGAGTGTCTTCTGCAT
GAATATGAAGATCTGGTACCCATCCGTGATACATTGAGGCTGTTCCCTGGGGGTCGTTACCTTCCA
CGAGCAAAACACGTAGCCCCTTCAGAGCCAGATCCTGAGCAAGATGAACAGAAACTGAGGTTTTG
TAAACGCCACCTTTATGGGCAGCAACCCCGATCACCGGTGGAAATACGTCTTCAGCACGTCGCAAT
CGCGTACCAAACACATCACGCATATGATTAATTTGTTCAATTGTATAACCAACACGTTGCTCAACC
CGTCCTCGAATTTCCATATCCGGGTGCGGTAGTCGCCCTGCTTTCTCGGCATCTCTGATAGCCTGAG
AAGAAACCCCAACTAAATCCGCTGCTTCACCTATTCTCCAGCGCCGGGTTATTTTCCTCGCTTCCGG
GCTGTCATCATTAAACTGTGCAATGGCGATAGCCTTCGTCATTTCATGACCAGCGTTTATGCACTG
GTTAAGTGTTTCCATGAGTTTCATTCTGAACATCCTTTAATCATTGCTTTGCGTTTTTTTATTAAATC
TTGCAATTTACTGCAAAGCAACAACAAAATCGCAAAGTCATCAAAAAACCGCAAAGTTGTTTAAA
ATAAGAGCAACACTACAAAAGGAGATAAGAAGAGCACATACCTCAGTCACTTATTATCACTAGCG
CTCGCCGCAGCCGTGTAACCGAGCATAGCGAGCGAACTGGCGAGGAAGCAAAGAAGAACTGTTCT
GTCAGATAGCTCTTACGCTCAGCGCAAGAAGAAATATCCACCGTGGGAAAAACTCCAGGTAGAGG
TACACACGCGGATAGCCAATTCAGAGTAATAAACTGTGATAATCAACCCTCATCAATGATGACGA
ACTAACCCCCGATATCAGGTCACATGACGAAGGGAAAGAGAAGGAAATCAACTGTGACAAACTGC
CCTCAAATTTGGCTTCCTTAAAAATTACAGTTCAAAAAGTATGAGAAAATCCATGCAGGCTGAAGG
AAACAGCAAAACTGTGACAAATTACCCTCAGTAGGTCAGAACAAATGTGACGAACCACCCTCAAA
TCTGTGACAGATAACCCTCAGACTATCCTGTCGTCATGGAAGTGATATCGCGGAAGGAAAATACG
ATATGAGTCGTCTGGCGGCCTTTCTTTTTCTCAATGTATGAGAGGCGCATTGGAGTTCTGCTGTTGA
TCTCATTAACACAGACCTGCAGGAAGCGGCGGCGGAAGTCAGGCATACGCTGGTAACTTTGAGGC
AGCTGGTAACGCTCTATGATCCAGTCGATTTTCAGAGAGACGATGCCTGAGCCATCCGGCTTACGA
TACTGACACAGGGATTCGTATAAACGCATGGCATACGGATTGGTGATTTCTTTTGTTTCACTAAGC
CGAAACTGCGTAAACCGGTTCTGTAACCCGATAAAGAAGGGAATGAGATATGGGTTGATATGTAC
ACTGTAAAGCCCTCTGGATGGACTGTGCGCACGTTTGATAAACCAAGGAAAAGATTCATAGCCTTT
TTCATCGCCGGCATCCTCTTCAGGGCGATAAAAAACCACTTCCTTCCCCGCGAAACTCTTCAATGC
CTGCCGTATATCCTTACTGGCTTCCGCAGAGGTCAATCCGAATATTTCAGCATATTTAGCAACATG
GATCTCGCAGATACCGTCATGTTCCTGTAGGGTGCCATCAGATTTTCTGATCTGGTCAACGAACAG
ATACAGCATACGTTTTTGATCCCGGGAGAGACTATATGCCGCCTCAGTGAGGTCGTTTGACTGGAC
GATTCGCGGGCTATTTTTACGTTTCTTGTGATTGATAACCGCTGTTTCCGCCATGACAGATCCATGT
GAAGTGTGACAAGTTTTTAGATTGTCACACTAAATAAAAAAGAGTCAATAAGCAGGGATAACTTT
GTGAAAAAACAGCTTCTTCTGAGGGCAATTTGTCACAGGGTTAAGGGCAATTTGTCACAGACAGG
ACTGTCATTTGAGGGTGATTTGTCACACTGAAAGGGCAATTTGTCACAACACCTTCTCTAGAACCA
GCATGGATAAAGGCCTACAAGGCGCTCTAAAAAAGAAGATCTAAAAACTATAAAAAAAATAATTA
TAAAAATATCCCCGTGGATAAGTGGATAACCCCAAGGGAAGTTTTTTCAGGCATCGTGTGTAAGCA
GAATATATAAGTGCTGTTCCCTGGTGCTTCCTCGCTCACTCGAGCTACGGGGTCTGACGCTCAGTG
GAACGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCATAGTTGCCTGA
CTGCCCGTCGTGTAGATAACTACGATACGGGAGGGCTTACCATCTGGCCCCAGTGCTGCAATGATA
CCGCGGGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGTTACATGATC
CCCCATGTTGTGAAAAAAAGCGGTTAGCTCCTTCGGTCCTCCGATCGTTGTCAGAAGTAAGTTGGC
CGCAGTGTTATCACTCATGCTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACCTGG
CGGCCGCTTG
*T7 promoter + filler DNA reporter plasmid
(SEQ ID NO: 63)
ACGAAAGGGCCTCGTGATACGCCTATTTTTATAGGTTAATGTCATGATAATAATGGTTTCTTAGAC
GTCAGGTGGCACTTTTCGGGGAAATGTGCCCGGTTAACGTGCCGGCACGGCCTGGGTAACCAGGT
ATTTTGTCCACATAACCGTGCGCAAAATGTTGTGGATAAGCAGGACACAGCAGCAATCCACAGCA
GGCATACAACCGCACACCGAGGTTACTCCGTTCTACAGGTTACGACGACATGTCAATACTTGCCCT
TGACAGGCATTGATGGAATCGTAGTCTCACGCTGATAGTCTGATCGACAATACAAGTGGGACCGT
GGTCCCAGACCGATAATCAGACCGACAACACGAGTGGGATCGTGGTCCCAGACTAATAATCAGAC
CGACGATACGAGTGGGACCGTGGTCCCAGACTAATAATCAGACCGACGATACGAGTGGGACCGTG
GTTCCAGACTAATAATCAGACCGACGATACGAGTGGGACCGTGGTCCCAGACTAATAATCAGACC
GACGATACGAGTGGGACCATGGTCCCAGACTAATAATCAGACCGACGATACGAGTGGGACCGTGG
TCCCAGTCTGATTATCAGACCGACGATACGAGTGGGACCGTGGTCCCAGACTAATAATCAGACCG
ACGATACGAGTGGGACCGTGGTCCCAGACTAATAATCAGACCGACGATACGAGTGGGACCGTGGT
CCCAGTCTGATTATCAGACCGACGATACAAGTGGAACAGTGGGCCCAGAGAGAATATTCAGGCCA
GTTATGCTTTCTGGCCTGTAACAAAGGACATTAAGTAAAGACAGATAAACGTAGACTAAAACGTG
GTCGCATCAGGGTGCTGGCTTTTCAAGTTCCTTAAGAATGGCCTCAATTTTCTCTATACACTCAGTT
GGAACACGAGACCTGTCCAGGTTAAGCACCATTTTATCGCCCTTATACAATACTGTCGCTCCAGGA
GCAAACTGATGTCGTGAGCTTAAACTAGTTCTTGATGCAGATGACGTTTTAAGCACAGAAGTTAAA
AGAGTGATAACTTCTTCAGCTTCAAATATCACCCCAGCTTTTTTCTGCTCATGAAGGTTAGATGCCT
GCTGCTTAAGTAATTCCTCTTTATCTGTAAAGGCTTTTTGAAGTGCATCACCTGACCGGGCAGATA
GTTCACCGGGGTGAGAAAAAAGAGCAACAACTGATTTAGGCAATTTGGCGGTGTTGATACAGCGG
GTAATAATCTTACGTGAAATATTTTCCGCATCAGCCAGCGCAGAAATATTTCCAGCAAATTCATTC
TGCAATCGGCTTGCATAACGCTGACCACGTTCATAAGCACTTGTTGGGCGATAATCGTTACCCAAT
CTGGATAATGCAGCCATCTGCTCATCATCCAGCTCGCCAACCAGAACACGATAATCACTTTCGGTA
AGTGCAGCAGCTTTACGACGGCGACTCCCATCGGCAATTTCTATGACACCAGATACTCTTCGACCG
AACGCCGGTGTCTGTTGACCAGTCAGTAGAAAAGAAGGGATGAGATCATCCAGTGCGTCCTCAGT
AAGCAGCTCCTGGTCACGTTCATTACCTGACCATACCCGAGAGGTCTTCTCAACACTATCACCCCG
GAGCACTTCAAGAGTAAACTTCACATCCCGACCACATACAGGCAAAGTAATGGCATTACCGCGAG
CCATTACTCCTACGCGCGCAATTAACGAATCCACCATCGGGGCAGCTGGTGTCGATAACGAAGTAT
CTTCAACCGGTTGAGTATTGAGCGTATGTTTTGGAATAACAGGCGCACGCTTCATTATCTAATCTCC
CAGCGTGGTTTAATCAGACGATCGAAAATTTCATTGCAGACAGGTTCCCAAATAGAAAGAGCATTT
CTCCAGGCACCAGTTGAAGAGCGTTGATCAATGGCCTGTTCAAAAACAGTTCTCATCCGGATCTGA
CCTTTACCAACTTCATCCGTTTCACGTACAACATTTTTTAGAACCATGCTTCCCCAGGCATCCCGAA
TTTGCTCCTCCATCCACGGGGACTGAGAGCCATTACTATTGCTGTATTTGGTAAGCAAAATACGTA
CATCAGGCTCGAACCCTTTAAGATCAACGTTCTTGAGCAGATCACGAAGCATATCGAAAAACTGC
AGTGCGGAGGTGTAGTCAAACAACTCAGCAGGCGTGGGAACAATCAGCACATCAGCAGCACATAC
GACATTAATCGTGCCGATACCCAGGTTAGGCGCGCTGTCAATAACTATGACATCATAGTCATGAGC
AACAGTTTCAATGGCCAGTCGGAGCATCAGGTGTGGATCGGTGGGCAGTTTACCTTCATCAAATTT
GCCCATTAACTCAGTTTCAATACGGTGCAGAGCCAGACAGGAAGGAATAATGTCAAGCCCCGGCC
AGCAAGTGGGCTTTATTGCATAAGTGACATCGTCCTTTTCCCCAAGATAGAAAGGCAGGAGAGTGT
CTTCTGCATGAATATGAAGATCTGGTACCCATCCGTGATACATTGAGGCTGTTCCCTGGGGGTCGT
TACCTTCCACGAGCAAAACACGTAGCCCCTTCAGAGCCAGATCCTGAGCAAGATGAACAGAAACT
GAGGTTTTGTAAACGCCACCTTTATGGGCAGCAACCCCGATCACCGGTGGAAATACGTCTTCAGCA
CGTCGCAATCGCGTACCAAACACATCACGCATATGATTAATTTGTTCAATTGTATAACCAACACGT
TGCTCAACCCGTCCTCGAATTTCCATATCCGGGTGCGGTAGTCGCCCTGCTTTCTCGGCATCTCTGA
TAGCCTGAGAAGAAACCCCAACTAAATCCGCTGCTTCACCTATTCTCCAGCGCCGGGTTATTTTCC
TCGCTTCCGGGCTGTCATCATTAAACTGTGCAATGGCGATAGCCTTCGTCATTTCATGACCAGCGTT
TATGCACTGGTTAAGTGTTTCCATGAGTTTCATTCTGAACATCCTTTAATCATTGCTTTGCGTTTTTT
TATTAAATCTTGCAATTTACTGCAAAGCAACAACAAAATCGCAAAGTCATCAAAAAACCGCAAAG
TTGTTTAAAATAAGAGCAACACTACAAAAGGAGATAAGAAGAGCACATACCTCAGTCACTTATTA
TCACTAGCGCTCGCCGCAGCCGTGTAACCGAGCATAGCGAGCGAACTGGCGAGGAAGCAAAGAA
GAACTGTTCTGTCAGATAGCTCTTACGCTCAGCGCAAGAAGAAATATCCACCGTGGGAAAAACTC
CAGGTAGAGGTACACACGCGGATAGCCAATTCAGAGTAATAAACTGTGATAATCAACCCTCATCA
ATGATGACGAACTAACCCCCGATATCAGGTCACATGACGAAGGGAAAGAGAAGGAAATCAACTGT
GACAAACTGCCCTCAAATTTGGCTTCCTTAAAAATTACAGTTCAAAAAGTATGAGAAAATCCATGC
AGGCTGAAGGAAACAGCAAAACTGTGACAAATTACCCTCAGTAGGTCAGAACAAATGTGACGAAC
CACCCTCAAATCTGTGACAGATAACCCTCAGACTATCCTGTCGTCATGGAAGTGATATCGCGGAAG
GAAAATACGATATGAGTCGTCTGGCGGCCTTTCTTTTTCTCAATGTATGAGAGGCGCATTGGAGTT
CTGCTGTTGATCTCATTAACACAGACCTGCAGGAAGCGGCGGCGGAAGTCAGGCATACGCTGGTA
ACTTTGAGGCAGCTGGTAACGCTCTATGATCCAGTCGATTTTCAGAGAGACGATGCCTGAGCCATC
CGGCTTACGATACTGACACAGGGATTCGTATAAACGCATGGCATACGGATTGGTGATTTCTTTTGT
TTCACTAAGCCGAAACTGCGTAAACCGGTTCTGTAACCCGATAAAGAAGGGAATGAGATATGGGT
TGATATGTACACTGTAAAGCCCTCTGGATGGACTGTGCGCACGTTTGATAAACCAAGGAAAAGATT
CATAGCCTTTTTCATCGCCGGCATCCTCTTCAGGGCGATAAAAAACCACTTCCTTCCCCGCGAAAC
TCTTCAATGCCTGCCGTATATCCTTACTGGCTTCCGCAGAGGTCAATCCGAATATTTCAGCATATTT
AGCAACATGGATCTCGCAGATACCGTCATGTTCCTGTAGGGTGCCATCAGATTTTCTGATCTGGTC
AACGAACAGATACAGCATACGTTTTTGATCCCGGGAGAGACTATATGCCGCCTCAGTGAGGTCGTT
TGACTGGACGATTCGCGGGCTATTTTTACGTTTCTTGTGATTGATAACCGCTGTTTCCGCCATGACA
GATCCATGTGAAGTGTGACAAGTTTTTAGATTGTCACACTAAATAAAAAAGAGTCAATAAGCAGG
GATAACTTTGTGAAAAAACAGCTTCTTCTGAGGGCAATTTGTCACAGGGTTAAGGGCAATTTGTCA
CAGACAGGACTGTCATTTGAGGGTGATTTGTCACACTGAAAGGGCAATTTGTCACAACACCTTCTC
TAGAACCAGCATGGATAAAGGCCTACAAGGCGCTCTAAAAAAGAAGATCTAAAAACTATAAAAA
AAATAATTATAAAAATATCCCCGTGGATAAGTGGATAACCCCAAGGGAAGTTTTTTCAGGCATCGT
GTGTAAGCAGAATATATAAGTGCTGTTCCCTGGTGCTTCCTCGCTCACTCGAGCTACGGGGTCTGA
CGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATA
GTTGCCTGACTGCCCGTCGTGTAGATAACTACGATACGGGAGGGCTTACCATCTGGCCCCAGTGCT
GCAATGATACCGCGGGACCCACGCTCACCGGCTCCAGATTTATCAGCAATAAACCAGCCAGCCGG
AAGGGCCGAGCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCAAGGCGAGT
TACATGATCCCCCATGTTGTGAAAAAAAGCGGTTAGCTCCTTCGGTCCTCCGATCGTTGTCAGAAG
TAAGTTGGCCGCAGTGTTATCACTCATGCTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAA
AGTGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAAGT
GCCACCTGGCGGCCGCCTTTCAGCAAAAAACCCCGCGAGACCCCCGAAGAGGCCCCGCGGGGTTA
TGCTAGGTCGACGGAGCTCGAATTCTAATACGACTCACTATAGGGAGACCCAAGCTGGCTTGACA
ATTAATCATCGGCTCGAAGCTTGTTGACAATTAATCATCGGCATAGTATATCGGCATAGTATAATA
CGACAAGGTGAGGAACTAAACCACGGGATCGGCCATTGAACAAGATGGATTGCACGCAGGTTCTC
CGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGT
GCCCTGAATGAACTGCAGGACGAGGCAGCGCGGCTATCGTGGCTGGCCACGACGGGCGTTCCTTG
CGCAGCTGTGCTCGACGTTGTCACTGAAGCGGGAAGGGACTGGCTGCTATTGGGCGAAGTGCCGG
GGCAGGATCTCCTGTCATCTCACCTTGCTCCTGCCGAGAAAGTATCCATCATGGCTGATGCAATGC
GGCGGCTGCATACGCTTGATCCGGCTACCTGCCCATTCGACCACCAAGCGAAACATCGCATCGAGC
GAGCACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGATGATCTCGTCGTG
ACCCATGGCGATGCCTGCTTGCCGAATATCATGGTGGAAAATGGCCGCTTTTCTGGATTCATCGAC
TGTGGCCGGCTGGGTGTGGCGGACCGCTATCAGGACATAGCGTTGGCTACCCGTGATATTGCTGAA
GAGCTTGGCGGCGAATGGGCTGACCGCTTCCTCGTGCTTTACGGTATCGCCGCTCCCGATTCGCAG
CGCATCGCCTTCTATCGCCTTCTTGACGAGTTCTTCTGAGGGGATCAATTCTCTAGAGCTTAATTAA
CGCAGCCTGAATGGCGAATAGAAGTTTAAACGCTAGATTGGACAATGGCGAGCCTAGTCTCCCAC
GGCGATCTTGCCGCCCTTCTTGGCCTTAATGAGAATCTCGCGGATCTTGCGGGCGTCCAACTTGCC
GGTCAGTCCTTTAGGCACCTCGTCCACGAACACAACACCACCGCGCAGCTTCTTGGCGGTTGTAAC
CTGGCTGGCCACATAGTCCACGATCTCCTTCTCGGTCATGGTTTTACCGTGTTCCAGCACGACGACT
GCGGCGGGCAGCTCGCCGGCATCGTCGTCGGGCAGGCCGGCGACCCCGGCGTCGAAGATGTTGGG
GTGTTGCAGCAGGATGCTCTCCAGTTCGGCTGGGGCTACCTGGTAGCCCTTGTATTTGATCAGGCT
CTTCAGCCGGTCCACGATGAAGAAGTGCTCGTCCTCGTCCCAGTAGGCGATGTCGCCGCTGTGCAG
CCAGCCGTCCTTGTCGATGAGAGCGTTTGTAGCCTCGGGGTTGTTAACGTAGCCGCTCATGATCAT
GGGGCCACGGACGCACAGCTCGCCGCGCTGGTTCACACCCAGTGTCTTACCGGTGTCCAAGTCCAC
CACCTTAGCCTCGAAGAAGGGCACCACCTTGCCTACTGCGCCAGGCTTGTCGTCCCCTTCGGGGGT
GATCAGAATGGCGCTGGTTGTTTCTGTCAGGCCGTAGCCCTGGCGGATGCCTGGTAGGTGGAAGCG
TTTGGCCACGGCCTCACCTACCTCCTTGCTGAGCGGCGCCCCGCCGCTGGCGATCTCGTGCAAGTT
GCTTAGGTCGTACTTGTCGATGAGAGTGCTCTTAGCGAAGAAGCTAAATAGTGTGGGCACCAGCA
GGGCAGATTGAATCTTATAGTCTTGCAAGCTGCGCAAGAATAGCTCCTCCTCGAAGCGGTACATGA
GCACGACCCGAAAGCCGCAGATCAAGTAGCCCAGCGTGGTGAACATGCCGAAGCCGTGGTGAAAT
GGCACCACGCTGAGGATAGCGGTGTCGGGGATGATCTGGTTGCCGAAGATGGGGTCGCGGGCATG
ACTGAATCGGACACAAGCGGTGCGGTGCGGTAGGGCTACGCCCTTGGGCAATCCGGTACTGCCAC
TACTGTTCATGATCAGGGCGATGGTTTTGTCCCGGTCGAAGCTCTCGGGCACGAAGTCGTACTCGT
TGAAGCCGGGTGGCAAATGGGAAGTCACGAAGGTGTACATGCTTTGGAAGCCCTGGTAGTCGGTC
TTGCTATCCATGATGATGATCTTTTGTATGATCGGTAGCTTCTTTTGCACGTTGAGGATCTTTTGCA
GCCCTTTCTTGCTCACGAATACGACGGTGGGCTGGCTGATGCCCATGCTGTTCAGCAGCTCGCGCT
CGTTGTAGATGTCGTTAGCTGGGGCCACAGCCACACCGATGAACAGGGCACCCAACACGGGCATG
AAGAACTGCAAGCTATTCTCGCTGCACACCACGATCCGATGGTTTGTATTCAGCCCATAGCGCTTC
ATAGCTTCTGCCAGCCGAACGCTCATCTCGAAGTACTCGGCGTAGGTAATGTCCACCTCGATATGT
GCGTCGGTAAAGGCGATGGTGCCGGGCACCAGGGCGTAGCGCTTCATGGCTTTGTGCAGCTGCTC
GCCGGCGGTCCCGTCTTCGAGTGGGTAGAATGGCGCTGGGCCCTTCTTAATGTTTTTGGCATCTTCC
ATGGTGGTGAATTCCACCACACTGGACTAGTGGATCCTAGGGATGTTTTGGCTCCATATGATCACT
ACAAAGACACCAGAACAGGTGTTGTAGTTGGACCAGATTCCAACCGATCCTTGACAGCTTATCATC
GATAAGCTTTAATGCGGTAGTTTATCACAGTTGCTAACGCAGTCAGGCACCGTGTACGAATAGTTC
GACAAAGATCGCATTGGTAATTACGTTACTCGATGCCATGGGGATTGGCCTTATCATGCCAGTCTT
GCCAACGTTATTACGTGAATTTATTGCTTCGGAAGATATCGCTAACCACTTTGGCGTATTGCTTGCA
CTTTATGCGTTAATGCAGGTTATCTTTGCTCCTTGGCTTGGAAAAATGTCTGACCGATTTGGTCGGC
GCCCAGTGCTGTTGTTGTCATTAATAGGCGCATCGCTGGATTACTTATTGCTGGCTTTTTCAAGTGC
GCTTTGGATGCTGTATTTAGGCCGTTTGCTTTCAGGGATCACAGGAGCTACTGGGGCTGTCGCGGC
ATCGGTCATTGCCGATACCACCTCAGCTTCTCAACGCGTGAAGTGGTTCGGTTGGTTAGGGGCAAG
TTTTGGGCTTGGTTTAATAGCGGGGCCTATTATTGGTGGTTTTGCAGGAGAGATTTCACCGCATAGT
CCCTTTTTTATCGCTGCGTTGCTAAATATTGTCACTTTCCTTGTGGTTATGTTTTGGTTCCGTGAAAC
CAAAAATACACGTGATAATACAGATACCGAAGTAGGGGTTGAGACGCAATCGAATTCGGTATACA
TCACTTTATTTAAAACGATGCCCATTTTGTTGATTATTTATTTTTCAGCGCAATTGATAGGCCAAAT
TCCCGCAACGGTGTGGGTGCTATTTACCGAAAATCGTTTTGGATGGAATAGCATGATGGTTGGCTT
TTCATTAGCGGGTCTTGGTCTTTTACACTCAGTATTCCAAGCCTTTGTGGCAGGAAGAATAGCCACT
AAATGGGGCGAAAAAACGGCAGTACTGCTCGGATTTATTGCAGATAGTAGTGCATTTGCCTTTTTA
GCGTTTATATCTGAAGGTTGGTTAGTTTTCCCTGTTTTAATTTTATTGGCTGGTGGTGGGATCGCTTT
ACCTGCATTACAGGGAGTGATGTCTATCCAAACAAAGAGTCATCAGCAAGGTGCTTTACAGGGAT
TATTGGTGAGCCTTACCAATGCAACCGGTGTTATTGGCCCATTACTGTTTGCTGTTATTTATAATCA
TTCACTACCAATTTGGGATGGCTGGATTTGGATTATTGGTTTAGCGTTTTACTGTATTATTATCCTG
CTATCGATGACCTTCATGTTAACCCCTCAAGCTCAGGGGAGTAAACAGGAGACAAGTGCTTAGTG
ATCCAATTCTTGAAG
*T7 promoter + terminators reporter plasmid
(SEQ ID NO: 64)
ACGAAAGGGCCTCGTGATACGCCTATTTTTATAGGTTAATGTCATGATAATAATGGTTTCTTAGAC
GTCAGGTGGCACTTTTCGGGGAAATGTGCCCGGTTAACGTGCCGGCACGGCCTGGGTAACCAGGT
ATTTTGTCCACATAACCGTGCGCAAAATGTTGTGGATAAGCAGGACACAGCAGCAATCCACAGCA
GGCATACAACCGCACACCGAGGTTACTCCGTTCTACAGGTTACGACGACATGTCAATACTTGCCCT
TGACAGGCATTGATGGAATCGTAGTCTCACGCTGATAGTCTGATCGACAATACAAGTGGGACCGT
GGTCCCAGACCGATAATCAGACCGACAACACGAGTGGGATCGTGGTCCCAGACTAATAATCAGAC
CGACGATACGAGTGGGACCGTGGTCCCAGACTAATAATCAGACCGACGATACGAGTGGGACCGTG
GTTCCAGACTAATAATCAGACCGACGATACGAGTGGGACCGTGGTCCCAGACTAATAATCAGACC
GACGATACGAGTGGGACCATGGTCCCAGACTAATAATCAGACCGACGATACGAGTGGGACCGTGG
TCCCAGTCTGATTATCAGACCGACGATACGAGTGGGACCGTGGTCCCAGACTAATAATCAGACCG
ACGATACGAGTGGGACCGTGGTCCCAGACTAATAATCAGACCGACGATACGAGTGGGACCGTGGT
CCCAGTCTGATTATCAGACCGACGATACAAGTGGAACAGTGGGCCCAGAGAGAATATTCAGGCCA
GTTATGCTTTCTGGCCTGTAACAAAGGACATTAAGTAAAGACAGATAAACGTAGACTAAAACGTG
GTCGCATCAGGGTGCTGGCTTTTCAAGTTCCTTAAGAATGGCCTCAATTTTCTCTATACACTCAGTT
GGAACACGAGACCTGTCCAGGTTAAGCACCATTTTATCGCCCTTATACAATACTGTCGCTCCAGGA
GCAAACTGATGTCGTGAGCTTAAACTAGTTCTTGATGCAGATGACGTTTTAAGCACAGAAGTTAAA
AGAGTGATAACTTCTTCAGCTTCAAATATCACCCCAGCTTTTTTCTGCTCATGAAGGTTAGATGCCT
GCTGCTTAAGTAATTCCTCTTTATCTGTAAAGGCTTTTTGAAGTGCATCACCTGACCGGGCAGATA
GTTCACCGGGGTGAGAAAAAAGAGCAACAACTGATTTAGGCAATTTGGCGGTGTTGATACAGCGG
GTAATAATCTTACGTGAAATATTTTCCGCATCAGCCAGCGCAGAAATATTTCCAGCAAATTCATTC
TGCAATCGGCTTGCATAACGCTGACCACGTTCATAAGCACTTGTTGGGCGATAATCGTTACCCAAT
CTGGATAATGCAGCCATCTGCTCATCATCCAGCTCGCCAACCAGAACACGATAATCACTTTCGGTA
AGTGCAGCAGCTTTACGACGGCGACTCCCATCGGCAATTTCTATGACACCAGATACTCTTCGACCG
AACGCCGGTGTCTGTTGACCAGTCAGTAGAAAAGAAGGGATGAGATCATCCAGTGCGTCCTCAGT
AAGCAGCTCCTGGTCACGTTCATTACCTGACCATACCCGAGAGGTCTTCTCAACACTATCACCCCG
GAGCACTTCAAGAGTAAACTTCACATCCCGACCACATACAGGCAAAGTAATGGCATTACCGCGAG
CCATTACTCCTACGCGCGCAATTAACGAATCCACCATCGGGGCAGCTGGTGTCGATAACGAAGTAT
CTTCAACCGGTTGAGTATTGAGCGTATGTTTTGGAATAACAGGCGCACGCTTCATTATCTAATCTCC
CAGCGTGGTTTAATCAGACGATCGAAAATTTCATTGCAGACAGGTTCCCAAATAGAAAGAGCATTT
CTCCAGGCACCAGTTGAAGAGCGTTGATCAATGGCCTGTTCAAAAACAGTTCTCATCCGGATCTGA
CCTTTACCAACTTCATCCGTTTCACGTACAACATTTTTTAGAACCATGCTTCCCCAGGCATCCCGAA
TTTGCTCCTCCATCCACGGGGACTGAGAGCCATTACTATTGCTGTATTTGGTAAGCAAAATACGTA
CATCAGGCTCGAACCCTTTAAGATCAACGTTCTTGAGCAGATCACGAAGCATATCGAAAAACTGC
AGTGCGGAGGTGTAGTCAAACAACTCAGCAGGCGTGGGAACAATCAGCACATCAGCAGCACATAC
GACATTAATCGTGCCGATACCCAGGTTAGGCGCGCTGTCAATAACTATGACATCATAGTCATGAGC
AACAGTTTCAATGGCCAGTCGGAGCATCAGGTGTGGATCGGTGGGCAGTTTACCTTCATCAAATTT
GCCCATTAACTCAGTTTCAATACGGTGCAGAGCCAGACAGGAAGGAATAATGTCAAGCCCCGGCC
AGCAAGTGGGCTTTATTGCATAAGTGACATCGTCCTTTTCCCCAAGATAGAAAGGCAGGAGAGTGT
CTTCTGCATGAATATGAAGATCTGGTACCCATCCGTGATACATTGAGGCTGTTCCCTGGGGGTCGT
TACCTTCCACGAGCAAAACACGTAGCCCCTTCAGAGCCAGATCCTGAGCAAGATGAACAGAAACT
GAGGTTTTGTAAACGCCACCTTTATGGGCAGCAACCCCGATCACCGGTGGAAATACGTCTTCAGCA
CGTCGCAATCGCGTACCAAACACATCACGCATATGATTAATTTGTTCAATTGTATAACCAACACGT
TGCTCAACCCGTCCTCGAATTTCCATATCCGGGTGCGGTAGTCGCCCTGCTTTCTCGGCATCTCTGA
TAGCCTGAGAAGAAACCCCAACTAAATCCGCTGCTTCACCTATTCTCCAGCGCCGGGTTATTTTCC
TCGCTTCCGGGCTGTCATCATTAAACTGTGCAATGGCGATAGCCTTCGTCATTTCATGACCAGCGTT
TATGCACTGGTTAAGTGTTTCCATGAGTTTCATTCTGAACATCCTTTAATCATTGCTTTGCGTTTTTT
TATTAAATCTTGCAATTTACTGCAAAGCAACAACAAAATCGCAAAGTCATCAAAAAACCGCAAAG
TTGTTTAAAATAAGAGCAACACTACAAAAGGAGATAAGAAGAGCACATACCTCAGTCACTTATTA
TCACTAGCGCTCGCCGCAGCCGTGTAACCGAGCATAGCGAGCGAACTGGCGAGGAAGCAAAGAA
GAACTGTTCTGTCAGATAGCTCTTACGCTCAGCGCAAGAAGAAATATCCACCGTGGGAAAAACTC
CAGGTAGAGGTACACACGCGGATAGCCAATTCAGAGTAATAAACTGTGATAATCAACCCTCATCA
ATGATGACGAACTAACCCCCGATATCAGGTCACATGACGAAGGGAAAGAGAAGGAAATCAACTGT
GACAAACTGCCCTCAAATTTGGCTTCCTTAAAAATTACAGTTCAAAAAGTATGAGAAAATCCATGC
AGGCTGAAGGAAACAGCAAAACTGTGACAAATTACCCTCAGTAGGTCAGAACAAATGTGACGAAC
CACCCTCAAATCTGTGACAGATAACCCTCAGACTATCCTGTCGTCATGGAAGTGATATCGCGGAAG
GAAAATACGATATGAGTCGTCTGGCGGCCTTTCTTTTTCTCAATGTATGAGAGGCGCATTGGAGTT
CTGCTGTTGATCTCATTAACACAGACCTGCAGGAAGCGGCGGCGGAAGTCAGGCATACGCTGGTA
ACTTTGAGGCAGCTGGTAACGCTCTATGATCCAGTCGATTTTCAGAGAGACGATGCCTGAGCCATC
CGGCTTACGATACTGACACAGGGATTCGTATAAACGCATGGCATACGGATTGGTGATTTCTTTTGT
TTCACTAAGCCGAAACTGCGTAAACCGGTTCTGTAACCCGATAAAGAAGGGAATGAGATATGGGT
TGATATGTACACTGTAAAGCCCTCTGGATGGACTGTGCGCACGTTTGATAAACCAAGGAAAAGATT
CATAGCCTTTTTCATCGCCGGCATCCTCTTCAGGGCGATAAAAAACCACTTCCTTCCCCGCGAAAC
TCTTCAATGCCTGCCGTATATCCTTACTGGCTTCCGCAGAGGTCAATCCGAATATTTCAGCATATTT
AGCAACATGGATCTCGCAGATACCGTCATGTTCCTGTAGGGTGCCATCAGATTTTCTGATCTGGTC
AACGAACAGATACAGCATACGTTTTTGATCCCGGGAGAGACTATATGCCGCCTCAGTGAGGTCGTT
TGACTGGACGATTCGCGGGCTATTTTTACGTTTCTTGTGATTGATAACCGCTGTTTCCGCCATGACA
GATCCATGTGAAGTGTGACAAGTTTTTAGATTGTCACACTAAATAAAAAAGAGTCAATAAGCAGG
GATAACTTTGTGAAAAAACAGCTTCTTCTGAGGGCAATTTGTCACAGGGTTAAGGGCAATTTGTCA
CAGACAGGACTGTCATTTGAGGGTGATTTGTCACACTGAAAGGGCAATTTGTCACAACACCTTCTC
TAGAACCAGCATGGATAAAGGCCTACAAGGCGCTCTAAAAAAGAAGATCTAAAAACTATAAAAA
AAATAATTATAAAAATATCCCCGTGGATAAGTGGATAACCCCAAGGGAAGTTTTTTCAGGCATCGT
GTGTAAGCAGAATATATAAGTGCTGTTCCCTGGTGCTTCCTCGCTCACTCGAGCTACGGGGTCTGA
CGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATA
GTTGCCTGACTGCCCGTCGTGTAGATAACTACGATACGGGAGGGCTTACCATCTGGCCCCAGTGCT
GCAATGATACCGCGGGACCCACGCTCACCGGCTCCAGATTTATCAGCAATAAACCAGCCAGCCGG
AAGGGCCGAGCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCAAGGCGAGT
TACATGATCCCCCATGTTGTGAAAAAAAGCGGTTAGCTCCTTCGGTCCTCCGATCGTTGTCAGAAG
TAAGTTGGCCGCAGTGTTATCACTCATGCTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAA
AGTGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAAGT
GCCACCTGGCGGCCGCCTTTCAGCAAAAAACCCCGCGAGACCCCCGAAGAGGCCCCGCGGGGTTA
TGCTAGGTCGACGGAGCTCGAATTCTAATACGACTCACTATAGGGAGACCCAAGCTGGCTTGACA
ATTAATCATCGGCTCGAAGCTTGTTGACAATTAATCATCGGCATAGTATATCGGCATAGTATAATA
CGACAAGGTGAGGAACTAAACCACGGGATCGGCCATTGAACAAGATGGATTGCACGCAGGTTCTC
CGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGT
GCCCTGAATGAACTGCAGGACGAGGCAGCGCGGCTATCGTGGCTGGCCACGACGGGCGTTCCTTG
CGCAGCTGTGCTCGACGTTGTCACTGAAGCGGGAAGGGACTGGCTGCTATTGGGCGAAGTGCCGG
GGCAGGATCTCCTGTCATCTCACCTTGCTCCTGCCGAGAAAGTATCCATCATGGCTGATGCAATGC
GGCGGCTGCATACGCTTGATCCGGCTACCTGCCCATTCGACCACCAAGCGAAACATCGCATCGAGC
GAGCACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGATGATCTCGTCGTG
ACCCATGGCGATGCCTGCTTGCCGAATATCATGGTGGAAAATGGCCGCTTTTCTGGATTCATCGAC
TGTGGCCGGCTGGGTGTGGCGGACCGCTATCAGGACATAGCGTTGGCTACCCGTGATATTGCTGAA
GAGCTTGGCGGCGAATGGGCTGACCGCTTCCTCGTGCTTTACGGTATCGCCGCTCCCGATTCGCAG
CGCATCGCCTTCTATCGCCTTCTTGACGAGTTCTTCTGAGGGGATCAATTCTCTAGAGCTTAATTAA
CGCAGCCTGAATGGCGAATAGAAGTTTAAACGCTAGACCTAGCATAACCCCGCGGGGCCTCTTCG
GGGGTCTCGCGGGGTTTTTTGCTGAAAGGCTAGACCTAGCATAACCCCGCGGGGCCTCTTCGGGGG
TCTCGCGGGGTTTTTTGCTGAAAGGCTAGACCTAGCATAACCCCGCGGGGCCTCTTCGGGGGTCTC
GCGGGGTTTTTTGCTGAAAGGCTAGACCTAGCATAACCCCGCGGGGCCTCTTCGGGGGTCTCGCGG
GGTTTTTTGCTGAAAGGCTAGACCTAGCATAACCCCGCGGGGCCTCTTCGGGGGTCTCGCGGGGTT
TTTTGCTGAAAGGCTAGGAAGTTTAAACGCTAGACCTAGCATAACCCCGCGGGGCCTCTTCGGGGG
TCTCGCGGGGTTTTTTGCTGAAAGGCTAGACCTAGCATAACCCCGCGGGGCCTCTTCGGGGGTCTC
GCGGGGTTTTTTGCTGAAAGGCTAGACCTAGCATAACCCCGCGGGGCCTCTTCGGGGGTCTCGCGG
GGTTTTTTGCTGAAAGGCTAGACCTAGCATAACCCCGCGGGGCCTCTTCGGGGGTCTCGCGGGGTT
TTTTGCTGAAAGGCTAGACCTAGCATAACCCCGCGGGGCCTCTTCGGGGGTCTCGCGGGGTTTTTT
GCTGAAAGGCTAGGATATATTGATCCTTGACAGCTTATCATCGATAAGCTTTAATGCGGTAGTTTA
TCACAGTTGCTAACGCAGTCAGGCACCGTGTACGAATAGTTCGACAAAGATCGCATTGGTAATTAC
GTTACTCGATGCCATGGGGATTGGCCTTATCATGCCAGTCTTGCCAACGTTATTACGTGAATTTATT
GCTTCGGAAGATATCGCTAACCACTTTGGCGTATTGCTTGCACTTTATGCGTTAATGCAGGTTATCT
TTGCTCCTTGGCTTGGAAAAATGTCTGACCGATTTGGTCGGCGCCCAGTGCTGTTGTTGTCATTAAT
AGGCGCATCGCTGGATTACTTATTGCTGGCTTTTTCAAGTGCGCTTTGGATGCTGTATTTAGGCCGT
TTGCTTTCAGGGATCACAGGAGCTACTGGGGCTGTCGCGGCATCGGTCATTGCCGATACCACCTCA
GCTTCTCAACGCGTGAAGTGGTTCGGTTGGTTAGGGGCAAGTTTTGGGCTTGGTTTAATAGCGGGG
CCTATTATTGGTGGTTTTGCAGGAGAGATTTCACCGCATAGTCCCTTTTTTATCGCTGCGTTGCTAA
ATATTGTCACTTTCCTTGTGGTTATGTTTTGGTTCCGTGAAACCAAAAATACACGTGATAATACAGA
TACCGAAGTAGGGGTTGAGACGCAATCGAATTCGGTATACATCACTTTATTTAAAACGATGCCCAT
TTTGTTGATTATTTATTTTTCAGCGCAATTGATAGGCCAAATTCCCGCAACGGTGTGGGTGCTATTT
ACCGAAAATCGTTTTGGATGGAATAGCATGATGGTTGGCTTTTCATTAGCGGGTCTTGGTCTTTTAC
ACTCAGTATTCCAAGCCTTTGTGGCAGGAAGAATAGCCACTAAATGGGGCGAAAAAACGGCAGTA
CTGCTCGGATTTATTGCAGATAGTAGTGCATTTGCCTTTTTAGCGTTTATATCTGAAGGTTGGTTAG
TTTTCCCTGTTTTAATTTTATTGGCTGGTGGTGGGATCGCTTTACCTGCATTACAGGGAGTGATGTC
TATCCAAACAAAGAGTCATCAGCAAGGTGCTTTACAGGGATTATTGGTGAGCCTTACCAATGCAAC
CGGTGTTATTGGCCCATTACTGTTTGCTGTTATTTATAATCATTCACTACCAATTTGGGATGGCTGG
ATTTGGATTATTGGTTTAGCGTTTTACTGTATTATTATCCTGCTATCGATGACCTTCATGTTAACCCC
TCAAGCTCAGGGGAGTAAACAGGAGACAAGTGCTTAGTGATCCAATTCTTGAAG
*T7 promoter + antisense T7 promoter reporter plasmid OR * Dual
opposing T7 promoters reporter plasmid
(SEQ ID NO: 65)
ACGAAAGGGCCTCGTGATACGCCTATTTTTATAGGTTAATGTCATGATAATAATGGTTTCTTAGAC
GTCAGGTGGCACTTTTCGGGGAAATGTGCCCGGTTAACGTGCCGGCACGGCCTGGGTAACCAGGT
ATTTTGTCCACATAACCGTGCGCAAAATGTTGTGGATAAGCAGGACACAGCAGCAATCCACAGCA
GGCATACAACCGCACACCGAGGTTACTCCGTTCTACAGGTTACGACGACATGTCAATACTTGCCCT
TGACAGGCATTGATGGAATCGTAGTCTCACGCTGATAGTCTGATCGACAATACAAGTGGGACCGT
GGTCCCAGACCGATAATCAGACCGACAACACGAGTGGGATCGTGGTCCCAGACTAATAATCAGAC
CGACGATACGAGTGGGACCGTGGTCCCAGACTAATAATCAGACCGACGATACGAGTGGGACCGTG
GTTCCAGACTAATAATCAGACCGACGATACGAGTGGGACCGTGGTCCCAGACTAATAATCAGACC
GACGATACGAGTGGGACCATGGTCCCAGACTAATAATCAGACCGACGATACGAGTGGGACCGTGG
TCCCAGTCTGATTATCAGACCGACGATACGAGTGGGACCGTGGTCCCAGACTAATAATCAGACCG
ACGATACGAGTGGGACCGTGGTCCCAGACTAATAATCAGACCGACGATACGAGTGGGACCGTGGT
CCCAGTCTGATTATCAGACCGACGATACAAGTGGAACAGTGGGCCCAGAGAGAATATTCAGGCCA
GTTATGCTTTCTGGCCTGTAACAAAGGACATTAAGTAAAGACAGATAAACGTAGACTAAAACGTG
GTCGCATCAGGGTGCTGGCTTTTCAAGTTCCTTAAGAATGGCCTCAATTTTCTCTATACACTCAGTT
GGAACACGAGACCTGTCCAGGTTAAGCACCATTTTATCGCCCTTATACAATACTGTCGCTCCAGGA
GCAAACTGATGTCGTGAGCTTAAACTAGTTCTTGATGCAGATGACGTTTTAAGCACAGAAGTTAAA
AGAGTGATAACTTCTTCAGCTTCAAATATCACCCCAGCTTTTTTCTGCTCATGAAGGTTAGATGCCT
GCTGCTTAAGTAATTCCTCTTTATCTGTAAAGGCTTTTTGAAGTGCATCACCTGACCGGGCAGATA
GTTCACCGGGGTGAGAAAAAAGAGCAACAACTGATTTAGGCAATTTGGCGGTGTTGATACAGCGG
GTAATAATCTTACGTGAAATATTTTCCGCATCAGCCAGCGCAGAAATATTTCCAGCAAATTCATTC
TGCAATCGGCTTGCATAACGCTGACCACGTTCATAAGCACTTGTTGGGCGATAATCGTTACCCAAT
CTGGATAATGCAGCCATCTGCTCATCATCCAGCTCGCCAACCAGAACACGATAATCACTTTCGGTA
AGTGCAGCAGCTTTACGACGGCGACTCCCATCGGCAATTTCTATGACACCAGATACTCTTCGACCG
AACGCCGGTGTCTGTTGACCAGTCAGTAGAAAAGAAGGGATGAGATCATCCAGTGCGTCCTCAGT
AAGCAGCTCCTGGTCACGTTCATTACCTGACCATACCCGAGAGGTCTTCTCAACACTATCACCCCG
GAGCACTTCAAGAGTAAACTTCACATCCCGACCACATACAGGCAAAGTAATGGCATTACCGCGAG
CCATTACTCCTACGCGCGCAATTAACGAATCCACCATCGGGGCAGCTGGTGTCGATAACGAAGTAT
CTTCAACCGGTTGAGTATTGAGCGTATGTTTTGGAATAACAGGCGCACGCTTCATTATCTAATCTCC
CAGCGTGGTTTAATCAGACGATCGAAAATTTCATTGCAGACAGGTTCCCAAATAGAAAGAGCATTT
CTCCAGGCACCAGTTGAAGAGCGTTGATCAATGGCCTGTTCAAAAACAGTTCTCATCCGGATCTGA
CCTTTACCAACTTCATCCGTTTCACGTACAACATTTTTTAGAACCATGCTTCCCCAGGCATCCCGAA
TTTGCTCCTCCATCCACGGGGACTGAGAGCCATTACTATTGCTGTATTTGGTAAGCAAAATACGTA
CATCAGGCTCGAACCCTTTAAGATCAACGTTCTTGAGCAGATCACGAAGCATATCGAAAAACTGC
AGTGCGGAGGTGTAGTCAAACAACTCAGCAGGCGTGGGAACAATCAGCACATCAGCAGCACATAC
GACATTAATCGTGCCGATACCCAGGTTAGGCGCGCTGTCAATAACTATGACATCATAGTCATGAGC
AACAGTTTCAATGGCCAGTCGGAGCATCAGGTGTGGATCGGTGGGCAGTTTACCTTCATCAAATTT
GCCCATTAACTCAGTTTCAATACGGTGCAGAGCCAGACAGGAAGGAATAATGTCAAGCCCCGGCC
AGCAAGTGGGCTTTATTGCATAAGTGACATCGTCCTTTTCCCCAAGATAGAAAGGCAGGAGAGTGT
CTTCTGCATGAATATGAAGATCTGGTACCCATCCGTGATACATTGAGGCTGTTCCCTGGGGGTCGT
TACCTTCCACGAGCAAAACACGTAGCCCCTTCAGAGCCAGATCCTGAGCAAGATGAACAGAAACT
GAGGTTTTGTAAACGCCACCTTTATGGGCAGCAACCCCGATCACCGGTGGAAATACGTCTTCAGCA
CGTCGCAATCGCGTACCAAACACATCACGCATATGATTAATTTGTTCAATTGTATAACCAACACGT
TGCTCAACCCGTCCTCGAATTTCCATATCCGGGTGCGGTAGTCGCCCTGCTTTCTCGGCATCTCTGA
TAGCCTGAGAAGAAACCCCAACTAAATCCGCTGCTTCACCTATTCTCCAGCGCCGGGTTATTTTCC
TCGCTTCCGGGCTGTCATCATTAAACTGTGCAATGGCGATAGCCTTCGTCATTTCATGACCAGCGTT
TATGCACTGGTTAAGTGTTTCCATGAGTTTCATTCTGAACATCCTTTAATCATTGCTTTGCGTTTTTT
TATTAAATCTTGCAATTTACTGCAAAGCAACAACAAAATCGCAAAGTCATCAAAAAACCGCAAAG
TTGTTTAAAATAAGAGCAACACTACAAAAGGAGATAAGAAGAGCACATACCTCAGTCACTTATTA
TCACTAGCGCTCGCCGCAGCCGTGTAACCGAGCATAGCGAGCGAACTGGCGAGGAAGCAAAGAA
GAACTGTTCTGTCAGATAGCTCTTACGCTCAGCGCAAGAAGAAATATCCACCGTGGGAAAAACTC
CAGGTAGAGGTACACACGCGGATAGCCAATTCAGAGTAATAAACTGTGATAATCAACCCTCATCA
ATGATGACGAACTAACCCCCGATATCAGGTCACATGACGAAGGGAAAGAGAAGGAAATCAACTGT
GACAAACTGCCCTCAAATTTGGCTTCCTTAAAAATTACAGTTCAAAAAGTATGAGAAAATCCATGC
AGGCTGAAGGAAACAGCAAAACTGTGACAAATTACCCTCAGTAGGTCAGAACAAATGTGACGAAC
CACCCTCAAATCTGTGACAGATAACCCTCAGACTATCCTGTCGTCATGGAAGTGATATCGCGGAAG
GAAAATACGATATGAGTCGTCTGGCGGCCTTTCTTTTTCTCAATGTATGAGAGGCGCATTGGAGTT
CTGCTGTTGATCTCATTAACACAGACCTGCAGGAAGCGGCGGCGGAAGTCAGGCATACGCTGGTA
ACTTTGAGGCAGCTGGTAACGCTCTATGATCCAGTCGATTTTCAGAGAGACGATGCCTGAGCCATC
CGGCTTACGATACTGACACAGGGATTCGTATAAACGCATGGCATACGGATTGGTGATTTCTTTTGT
TTCACTAAGCCGAAACTGCGTAAACCGGTTCTGTAACCCGATAAAGAAGGGAATGAGATATGGGT
TGATATGTACACTGTAAAGCCCTCTGGATGGACTGTGCGCACGTTTGATAAACCAAGGAAAAGATT
CATAGCCTTTTTCATCGCCGGCATCCTCTTCAGGGCGATAAAAAACCACTTCCTTCCCCGCGAAAC
TCTTCAATGCCTGCCGTATATCCTTACTGGCTTCCGCAGAGGTCAATCCGAATATTTCAGCATATTT
AGCAACATGGATCTCGCAGATACCGTCATGTTCCTGTAGGGTGCCATCAGATTTTCTGATCTGGTC
AACGAACAGATACAGCATACGTTTTTGATCCCGGGAGAGACTATATGCCGCCTCAGTGAGGTCGTT
TGACTGGACGATTCGCGGGCTATTTTTACGTTTCTTGTGATTGATAACCGCTGTTTCCGCCATGACA
GATCCATGTGAAGTGTGACAAGTTTTTAGATTGTCACACTAAATAAAAAAGAGTCAATAAGCAGG
GATAACTTTGTGAAAAAACAGCTTCTTCTGAGGGCAATTTGTCACAGGGTTAAGGGCAATTTGTCA
CAGACAGGACTGTCATTTGAGGGTGATTTGTCACACTGAAAGGGCAATTTGTCACAACACCTTCTC
TAGAACCAGCATGGATAAAGGCCTACAAGGCGCTCTAAAAAAGAAGATCTAAAAACTATAAAAA
AAATAATTATAAAAATATCCCCGTGGATAAGTGGATAACCCCAAGGGAAGTTTTTTCAGGCATCGT
GTGTAAGCAGAATATATAAGTGCTGTTCCCTGGTGCTTCCTCGCTCACTCGAGCTACGGGGTCTGA
CGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATA
GTTGCCTGACTGCCCGTCGTGTAGATAACTACGATACGGGAGGGCTTACCATCTGGCCCCAGTGCT
GCAATGATACCGCGGGACCCACGCTCACCGGCTCCAGATTTATCAGCAATAAACCAGCCAGCCGG
AAGGGCCGAGCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCAAGGCGAGT
TACATGATCCCCCATGTTGTGAAAAAAAGCGGTTAGCTCCTTCGGTCCTCCGATCGTTGTCAGAAG
TAAGTTGGCCGCAGTGTTATCACTCATGCTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAA
AGTGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAAGT
GCCACCTGGCGGCCGCCTTTCAGCAAAAAACCCCGCGAGACCCCCGAAGAGGCCCCGCGGGGTTA
TGCTAGGTCGACGGAGCTCGAATTCTAATACGACTCACTATAGGGAGACCCAAGCTGGCTTGACA
ATTAATCATCGGCTCGAAGCTTGTTGACAATTAATCATCGGCATAGTATATCGGCATAGTATAATA
CGACAAGGTGAGGAACTAAACCACGGGATCGGCCATTGAACAAGATGGATTGCACGCAGGTTCTC
CGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGT
GCCCTGAATGAACTGCAGGACGAGGCAGCGCGGCTATCGTGGCTGGCCACGACGGGCGTTCCTTG
CGCAGCTGTGCTCGACGTTGTCACTGAAGCGGGAAGGGACTGGCTGCTATTGGGCGAAGTGCCGG
GGCAGGATCTCCTGTCATCTCACCTTGCTCCTGCCGAGAAAGTATCCATCATGGCTGATGCAATGC
GGCGGCTGCATACGCTTGATCCGGCTACCTGCCCATTCGACCACCAAGCGAAACATCGCATCGAGC
GAGCACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGATGATCTCGTCGTG
ACCCATGGCGATGCCTGCTTGCCGAATATCATGGTGGAAAATGGCCGCTTTTCTGGATTCATCGAC
TGTGGCCGGCTGGGTGTGGCGGACCGCTATCAGGACATAGCGTTGGCTACCCGTGATATTGCTGAA
GAGCTTGGCGGCGAATGGGCTGACCGCTTCCTCGTGCTTTACGGTATCGCCGCTCCCGATTCGCAG
CGCATCGCCTTCTATCGCCTTCTTGACGAGTTCTTCTGAGGGGATCAATTCTCTAGAGCTTAATTAA
CGCAGCCTGAATGGCGAATAGAAGTTTAAACGCTAGCCAGCTTGGGTCTCCCTATAGTGAGTCGTA
TTATCGAGCTCCGTCGACCTAGCATAACCCCGCGGGGCCTCTTCGGGGGTCTCGCGGGGTTTTTTG
CTGAAAGGGATCCAATTCTTGAAG
*R6K-kan-ccdB
(SEQ ID NO: 66)
CCTCCCACACATAACCAGGAGGTCAGATTATGCAGTTTAAGGTTTACACCTATAAAAGAGAGAGC
CGTTATCGTCTGTTTGTGGATGTACAGAGTGATATTATTGACACGCCCGGGCGACGGATGGTGATC
CCCCTGGCCAGTGCACGTCTGCTGTCAGATAAAGTCTCCCGTGAACTTTACCCGGTGGTGCATATC
GGGGATGAAAGCTGGCGCATGATGACCACCGATATGGCCAGTGTGCCGGTCTCCGTTATCGGGGA
AGAAGTGGCTGATCTCAGCCACCGCGAAAATGACATCAAAAACGCCATTAACCTGATGTTCTGGG
GAATATAACCCAGAAGCTTAGCAAAAGCTAAAACCAGGAGCTATTTAATGGCAACAGTTAACCAG
CTGGTACGCAAACCACGTGCTCGCAAAGTTGCGAAAAGCAACGTGCCTGCGCTGGAAGCATGCCC
GCAAAAACGTGGCGTATGTACTCGTGTATATACTACCACTCCTAAAAAACCGAACTCCGCGCTGCG
TAAAGTATGCCGTGTTCGTCTGACTAACGGTTTCGAAGTGACTTCCTACATCGGTGGTGAAGGTCA
CAACCTGCAGGAGCACTCCGTGATCCTGATCCGTGGCGGTCGTGTTAAAGACCTCCCGGGTGTTCG
TTACCACACCGTACGTGGTGCGCTTGACTGCTCCGGCGTTAAAGACCGTAAGCAGGCTCGTTCCAA
GTATGGCGTGAAGCGTCCTAAGGCTTAATGGTTCGCCCGCCTAATGAGCGGGCTTTTTTTTGAATT
CTTTTTTAATTCGATCTGAAGATCAGCAGTTCAACCTGTTGATAGTACGTACTAAGCTCTCATGTTT
CACGTACTAAGCTCTCATGTTTAACGTACTAAGCTCTCATGTTTAACGAACTAAACCCTCATGGCT
AACGTACTAAGCTCTCATGGCTAACGTACTAAGCTCTCATGTTTCACGTACTAAGCTCTCATGTTTG
AACAATAAAATTAATATAAATCAGCAACTTAAATAGCCTCTAAGGTTTTAAGTTTTATAAGAAAAA
AAAGAATATATAAGGCTTTTAAAGCTTTTAAGGTTTAACGGTTGTGGACAACAAGCCAGGGATGT
AACGCACTGAGAAGCCCTTAGAGCCTCTCAAAGCAATTTTGAGTGACACAGGAACACTTAACGGC
TGACATGGGATCCCCCTCATCAGTGCCAACATAGTAAGCCAGTATACACTCCGCTAGCGCGGCCGC
CTCGAGTTTCGACCTGCAGCCTGTTGACAATTAATCATCGGCATAGTATATCGGCATAGTATAATA
CGACAAGGTGAGGAACTAAACCATGGGATCGGCCATTGAACAAGATGGATTGCACGCAGGTTCTC
CGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGT
GCCCTGAATGAACTGCAGGACGAGGCAGCGCGGCTATCGTGGCTGGCCACGACGGGCGTTCCTTG
CGCAGCTGTGCTCGACGTTGTCACTGAAGCGGGAAGGGACTGGCTGCTATTGGGCGAAGTGCCGG
GGCAGGATCTCCTGTCATCTCACCTTGCTCCTGCCGAGAAAGTATCCATCATGGCTGATGCAATGC
GGCGGCTGCATACGCTTGATCCGGCTACCTGCCCATTCGACCACCAAGCGAAACATCGCATCGAGC
GAGCACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGATGATCTCGTCGTG
ACCCATGGCGATGCCTGCTTGCCGAATATCATGGTGGAAAATGGCCGCTTTTCTGGATTCATCGAC
TGTGGCCGGCTGGGTGTGGCGGACCGCTATCAGGACATAGCGTTGGCTACCCGTGATATTGCTGAA
GAGCTTGGCGGCGAATGGGCTGACCGCTTCCTCGTGCTTTACGGTATCGCCGCTCCCGATTCGCAG
CGCATCGCCTTCTATCGCCTTCTTGACGAGTTCTTCTGAGGGGATCAATTCTCTAGAGCTCGCTGAT
CAGCCTCGACTGTACCGTTAGC
*R6K-AmilCP-kan-ccdB
(SEQ ID NO: 67)
CACATAACCAGGAGGTCAGATTATGCAGTTTAAGGTTTACACCTATAAAAGAGAGAGCCGTTATC
GTCTGTTTGTGGATGTACAGAGTGATATTATTGACACGCCCGGGCGACGGATGGTGATCCCCCTGG
CCAGTGCACGTCTGCTGTCAGATAAAGTCTCCCGTGAACTTTACCCGGTGGTGCATATCGGGGATG
AAAGCTGGCGCATGATGACCACCGATATGGCCAGTGTGCCGGTCTCCGTTATCGGGGAAGAAGTG
GCTGATCTCAGCCACCGCGAAAATGACATCAAAAACGCCATTAACCTGATGTTCTGGGGAATATA
ACCCAGAAGCTTAGCAAAAGCTAAAACCAGGAGCTATTTAATGGCAACAGTTAACCAGCTGGTAC
GCAAACCACGTGCTCGCAAAGTTGCGAAAAGCAACGTGCCTGCGCTGGAAGCATGCCCGCAAAAA
CGTGGCGTATGTACTCGTGTATATACTACCACTCCTAAAAAACCGAACTCCGCGCTGCGTAAAGTA
TGCCGTGTTCGTCTGACTAACGGTTTCGAAGTGACTTCCTACATCGGTGGTGAAGGTCACAACCTG
CAGGAGCACTCCGTGATCCTGATCCGTGGCGGTCGTGTTAAAGACCTCCCGGGTGTTCGTTACCAC
ACCGTACGTGGTGCGCTTGACTGCTCCGGCGTTAAAGACCGTAAGCAGGCTCGTTCCAAGTATGGC
GTGAAGCGTCCTAAGGCTTAATGGTTCGCCCGCCTAATGAGCGGGCTTTTTTTTGAATTCTTTTTTA
ATTCGATCTGAAGATCAGCAGTTCAACCTGTTGATAGTACGTACTAAGCTCTCATGTTTCACGTACT
AAGCTCTCATGTTTAACGTACTAAGCTCTCATGTTTAACGAACTAAACCCTCATGGCTAACGTACT
AAGCTCTCATGGCTAACGTACTAAGCTCTCATGTTTCACGTACTAAGCTCTCATGTTTGAACAATA
AAATTAATATAAATCAGCAACTTAAATAGCCTCTAAGGTTTTAAGTTTTATAAGAAAAAAAAGAAT
ATATAAGGCTTTTAAAGCTTTTAAGGTTTAACGGTTGTGGACAACAAGCCAGGGATGTAACGCACT
GAGAAGCCCTTAGAGCCTCTCAAAGCAATTTTGAGTGACACAGGAACACTTAACGGCTGACATGG
GATCCGAATTAAAAAAGAATTCAAAAAAAAGCCCGCTCATTAGGCGGGCGAACCAACCGGTTTAG
GCGACCACAGGTTTGCGTGCAATGGAAATTTCACACTGCTCAACCGAAGTGTAATCCTTGTTGTGA
TTGGTTACATCCAGTTTGCGGTCAACATAGTGATACCCTGGCATCTTCACAGGCTTCTTTGCCTTGT
AAGTAGTTTTAAATTCACACAAATAGTGACCGCCTCCTTCTAACTTCAGAGCCATAAAGTTGTTTC
CTAGCAGCATTCCATCTCGTGCAAAGAGACGCTCAGTGTTGGGTTCCCAGCCCTGTGTCTTCTTCTG
CATGACAGGTCCATTGGGAGGAAAGTTCAAACCAGAGAACTTGACATGGTAGATGAAACAGTTGC
CTTGGATGCTGGAATCATTGCTGACAGTACACACTGCACCATCTTCAAAGTTCATGATCCTCTCCC
ATGTATAGCCCTCCGGGAATGACTGCTTTACATAGTCAGGGATGTCTTCAGGGTACTTGGTGAATG
GTATGCTTCCGTACTGACACTGTGGTGATAAAATATCCCAAGCAAATGGCAGAGGTCCGCCCTTGG
TGACAGTGAGCTTTACCGTCTGCTCCCCCTCGTAGGGCTTACCTTTTCCATCGCCTTCGACCTCAAA
GTAGTGTCCATTGACCGTGCCTGACATATAAACCTTGTAGGTCATTTGTTTAGCGATCACACTCATC
TAGTATTTCTCCTCTTTAATTACTAGATCCACACATTATAGGTACAAAAAGACATTATACGAGCCG
GAAGCATAAAGTGTAAAGGTACCCATCAGTGCCAACATAGTAAGCCAGTATACACTCCGCTAGCG
CGGCCGCCTCGAGTTTCGACCTGCAGCCTGTTGACAATTAATCATCGGCATAGTATATCGGCATAG
TATAATACGACAAGGTGAGGAACTAAACCATGGGATCGGCCATTGAACAAGATGGATTGCACGCA
GGTTCTCCGGCCGCTTGGGTGGAGAGGCTATTCGGCTATGACTGGGCACAACAGACAATCGGCTG
CTCTGATGCCGCCGTGTTCCGGCTGTCAGCGCAGGGGCGCCCGGTTCTTTTTGTCAAGACCGACCT
GTCCGGTGCCCTGAATGAACTGCAGGACGAGGCAGCGCGGCTATCGTGGCTGGCCACGACGGGCG
TTCCTTGCGCAGCTGTGCTCGACGTTGTCACTGAAGCGGGAAGGGACTGGCTGCTATTGGGCGAAG
TGCCGGGGCAGGATCTCCTGTCATCTCACCTTGCTCCTGCCGAGAAAGTATCCATCATGGCTGATG
CAATGCGGCGGCTGCATACGCTTGATCCGGCTACCTGCCCATTCGACCACCAAGCGAAACATCGCA
TCGAGCGAGCACGTACTCGGATGGAAGCCGGTCTTGTCGATCAGGATGATCTGGACGAAGAGCAT
CAGGGGCTCGCGCCAGCCGAACTGTTCGCCAGGCTCAAGGCGCGCATGCCCGACGGCGATGATCT
CGTCGTGACCCATGGCGATGCCTGCTTGCCGAATATCATGGTGGAAAATGGCCGCTTTTCTGGATT
CATCGACTGTGGCCGGCTGGGTGTGGCGGACCGCTATCAGGACATAGCGTTGGCTACCCGTGATAT
TGCTGAAGAGCTTGGCGGCGAATGGGCTGACCGCTTCCTCGTGCTTTACGGTATCGCCGCTCCCGA
TTCGCAGCGCATCGCCTTCTATCGCCTTCTTGACGAGTTCTTCTGAGGGGATCAATTCTCTAGAGCT
CGCTGATCAGCCTCGACTGTACCGTTAGCCCTCCCA
*R6K-kan-ccdB-AmilCP
(SEQ ID NO: 68)
CACATAACCAGGAGGTCAGATTATGCAGTTTAAGGTTTACACCTATAAAAGAGAGAGCCGTTATC
GTCTGTTTGTGGATGTACAGAGTGATATTATTGACACGCCCGGGCGACGGATGGTGATCCCCCTGG
CCAGTGCACGTCTGCTGTCAGATAAAGTCTCCCGTGAACTTTACCCGGTGGTGCATATCGGGGATG
AAAGCTGGCGCATGATGACCACCGATATGGCCAGTGTGCCGGTCTCCGTTATCGGGGAAGAAGTG
GCTGATCTCAGCCACCGCGAAAATGACATCAAAAACGCCATTAACCTGATGTTCTGGGGAATATA
ACCCAGAAGCTTAGCAAAAGCTAAAACCAGGAGCTATTTAGGTACCTTTACACTTTATGCTTCCGG
CTCGTATAATGTCTTTTTGTACCTATAATGTGTGGATCTAGTAATTAAAGAGGAGAAATACTAGAT
GAGTGTGATCGCTAAACAAATGACCTACAAGGTTTATATGTCAGGCACGGTCAATGGACACTACTT
TGAGGTCGAAGGCGATGGAAAAGGTAAGCCCTACGAGGGGGAGCAGACGGTAAAGCTCACTGTC
ACCAAGGGCGGACCTCTGCCATTTGCTTGGGATATTTTATCACCACAGTGTCAGTACGGAAGCATA
CCATTCACCAAGTACCCTGAAGACATCCCTGACTATGTAAAGCAGTCATTCCCGGAGGGCTATACA
TGGGAGAGGATCATGAACTTTGAAGATGGTGCAGTGTGTACTGTCAGCAATGATTCCAGCATCCA
AGGCAACTGTTTCATCTACCATGTCAAGTTCTCTGGTTTGAACTTTCCTCCCAATGGACCTGTCATG
CAGAAGAAGACACAGGGCTGGGAACCCAACACTGAGCGTCTCTTTGCACGAGATGGAATGCTGCT
AGGAAACAACTTTATGGCTCTGAAGTTAGAAGGAGGCGGTCACTATTTGTGTGAATTTAAAACTAC
TTACAAGGCAAAGAAGCCTGTGAAGATGCCAGGGTATCACTATGTTGACCGCAAACTGGATGTAA
CCAATCACAACAAGGATTACACTTCGGTTGAGCAGTGTGAAATTTCCATTGCACGCAAACCTGTGG
TCGCCTAAACCGGTTGGTTCGCCCGCCTAATGAGCGGGCTTTTTTTTGAATTCTTTTTTAATTCGAT
CTGAAGATCAGCAGTTCAACCTGTTGATAGTACGTACTAAGCTCTCATGTTTCACGTACTAAGCTC
TCATGTTTAACGTACTAAGCTCTCATGTTTAACGAACTAAACCCTCATGGCTAACGTACTAAGCTCT
CATGGCTAACGTACTAAGCTCTCATGTTTCACGTACTAAGCTCTCATGTTTGAACAATAAAATTAA
TATAAATCAGCAACTTAAATAGCCTCTAAGGTTTTAAGTTTTATAAGAAAAAAAAGAATATATAAG
GCTTTTAAAGCTTTTAAGGTTTAACGGTTGTGGACAACAAGCCAGGGATGTAACGCACTGAGAAG
CCCTTAGAGCCTCTCAAAGCAATTTTGAGTGACACAGGAACACTTAACGGCTGACATGGGATCCCC
CTCATCAGTGCCAACATAGTAAGCCAGTATACACTCCGCTAGCGCGGCCGCCTCGAGTTTCGACCT
GCAGCCTGTTGACAATTAATCATCGGCATAGTATATCGGCATAGTATAATACGACAAGGTGAGGA
ACTAAACCATGGGATCGGCCATTGAACAAGATGGATTGCACGCAGGTTCTCCGGCCGCTTGGGTG
GAGAGGCTATTCGGCTATGACTGGGCACAACAGACAATCGGCTGCTCTGATGCCGCCGTGTTCCGG
CTGTCAGCGCAGGGGCGCCCGGTTCTTTTTGTCAAGACCGACCTGTCCGGTGCCCTGAATGAACTG
CAGGACGAGGCAGCGCGGCTATCGTGGCTGGCCACGACGGGCGTTCCTTGCGCAGCTGTGCTCGA
CGTTGTCACTGAAGCGGGAAGGGACTGGCTGCTATTGGGCGAAGTGCCGGGGCAGGATCTCCTGT
CATCTCACCTTGCTCCTGCCGAGAAAGTATCCATCATGGCTGATGCAATGCGGCGGCTGCATACGC
TTGATCCGGCTACCTGCCCATTCGACCACCAAGCGAAACATCGCATCGAGCGAGCACGTACTCGG
ATGGAAGCCGGTCTTGTCGATCAGGATGATCTGGACGAAGAGCATCAGGGGCTCGCGCCAGCCGA
ACTGTTCGCCAGGCTCAAGGCGCGCATGCCCGACGGCGATGATCTCGTCGTGACCCATGGCGATGC
CTGCTTGCCGAATATCATGGTGGAAAATGGCCGCTTTTCTGGATTCATCGACTGTGGCCGGCTGGG
TGTGGCGGACCGCTATCAGGACATAGCGTTGGCTACCCGTGATATTGCTGAAGAGCTTGGCGGCG
AATGGGCTGACCGCTTCCTCGTGCTTTACGGTATCGCCGCTCCCGATTCGCAGCGCATCGCCTTCTA
TCGCCTTCTTGACGAGTTCTTCTGAGGGGATCAATTCTCTAGAGCTCGCTGATCAGCCTCGACTGTA
CCGTTAGCCCTCCCA
*BBa_J23114 lacO MutaT7
(SEQ ID NO: 69)
ATTAACTGGCGAACTACTTACTCTAGCTTCCCGGCAACAATTAATAGACTGGATGGAGGCGGATAA
AGTTGCAGGACCACTTCTGCGCTCGGCCCTTCCGGCTGGCTGGTTTATTGCTGATAAATCTGGAGC
CGGTGAGCGTGGGTCTCGCGGTATCATTGCAGCACTGGGGCCAGATGGTAAGCCCTCCCGTATCGT
AGTTATCTACACGACGGGGAGTCAGGCAACTATGATGAACGAAATAGACAGATCGCTGAGATAGG
TGCCTCACTGATTAAGCATTGGTAACTGTCAGACCAAGTTTACTCATATATACTTTAGATTGATTTA
CGCGCCCTGTAGCGGCGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTCGCCACGTTCGCCGGCTTT
CCCCGTCAAGCTCTAAATCGGGGGCTCCCTTTAGGGTTCCGATTTAGTGCTTTACGGCACCTCGAC
CCCAAAAAACTTGATTTGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTTGAACAACACTCAACC
CTATCTCGGGCTATTCTTTTGATTTATAAGGGATTTTGCCGATTTCGGCCATTGGTTAAAAAATGAG
CTGATTTAACAAAAATTTAACGCGAATTTTAACAAAATATTAACGTTTACAATTTAAAAGGATCTA
GGTGAAGATCCTTTTTGATAATCTCATGACCAAAATCCCTTAACGTGAGTTTTCGTTCCACTGAGCG
TCAGACCCCGTAGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTCCTTCTAGTGTAGCCGTAGT
TAGGCCACCACTTCAAGAACTCTGTAGCACCGCCTACATACCTCGCTCTGCTAATCCTGTTACCAG
TCAGGCATTTGAGAAGCACACGGTCACACTGCTTCCGGTAGTCAATAAACCGGTAAACCAGCAAT
AGACATAAGCGGCTATTTAACGACCCTGCCCTGAACCGACGACCGGGTCGAATTTGCTTTCGAATT
TCTGCCATTCATCCGCTTATTATCACTTATTCAGGCGTAGCACCAGGCGTTTAAGGGCACCAATAA
CTGCCTTAAAAAAATTACGCCCCGCCCTGCCACTCATCGCAGTACTGTTGTAATTCATTAAGCATTC
TGCCGACATGGAAGCCATCACAGACGGCATGATGAACCTGAATCGCCAGCGGCATCAGCACCTTG
TCGCCTTGCGTATAATATTTGCCCATGGTGAAAACGGGGGCGAAGAAGTTGTCCATATTGGCCACG
TTTAAATCAAAACTGGTGAAACTCACCCAGGGATTGGCTGAGACGAAAAACATATTCTCAATAAA
CCCTTTAGGGAAATAGGCCAGGTTTTCACCGTAACACGCCACATCTTGCGAATATATGTGTAGAAA
CTGCCGGAAATCGTCGTGGTATTCACTCCAGAGCGATGAAAACGTTTCAGTTTGCTCATGGAAAAC
GGTGTAACAAGGGTGAACACTATCCCATATCACCAGCTCACCGTCTTTCATTGCCATACGGAATTC
CGGATGAGCATTCATCAGGCGGGCAAGAATGTGAATAAAGGCCGGATAAAACTTGTGCTTATTTTT
CTTTACGGTCTTTAAAAAGGCCGTAATATCCAGCTGAACGGTCTGGTTATAGGTACATTGAGCAAC
TGACTGAAATGCCTCAAAATGTTCTTTACGATGCCATTGGGATATATCAACGGTGGTATATCCAGT
GATTTTTTTCTCCATTTTAGCTTCCTTAGCTCCTGAAAATCTCGATAACTCAAAAAATACGCCCGGT
AGTGATCTTATTTCATTATGGTGAAAGTTGGAACCTCTTACGTGCCGATCAACGTCTCATTTTCGCC
AAAAGTTGGCCCAGGGCTTCCCGGTATCAACAGGGACACCAGGATTTATTTATTCTGCGAAGTGAT
CTTCCGTCACAGGTATTTATTCGGCGCAAAGTGCGTCGGGTGATGCTGCCAACTTACTGATTTAGT
GTATGATGGTGTTTTTGAGGTGCTCCAGTGGCTTCTGTTTCTATCAGCTGTCCCTCCTGTTCAGCTA
CTGACGGGGTGGTGCGTAACGGCAAAAGCACCGCCGGACATCAGCGCTAGCGGAGTGTATACTGG
CTTACTATGTTGGCACTGATGAGGGTGTCAGTGAAGTGCTTCATGTGGCAGGAGAAAAAAGGCTG
CACCGGTGCGTCAGCAGAATATGTGATACAGGATATATTCCGCTTCCTCGCTCACTGACTCGCTAC
GCTCGGTCGTTCGACTGCGGCGAGCGGAAATGGCTTACGAACGGGGCGGAGATTTCCTGGAAGAT
GCCAGGAAGATACTTAACAGGGAAGTGAGAGGGCCGCGGCAAAGCCGTTTTTCCATAGGCTCCGC
CCCCCTGACAAGCATCACGAAATCTGACGCTCAAATCAGTGGTGGCGAAACCCGACAGGACTATA
AAGATACCAGGCGTTTCCCCCTGGCGGCTCCCTCGTGCGCTCTCCTGTTCCTGCCTTTCGGTTTACC
GGTGTCATTCCGCTGTTATGGCCGCGTTTGTCTCATTCCACGCCTGACACTCAGTTCCGGGTAGGCA
GTTCGCTCCAAGCTGGACTGTATGCACGAACCCCCCGTTCAGTCCGACCGCTGCGCCTTATCCGGT
AACTATCGTCTTGAGTCCAACCCGGAAAGACATGCAAAAGCACCACTGGCAGCAGCCACTGGTAA
TTGATTTAGAGGAGTTAGTCTTGAAGTCATGCGCCGGTTAAGGCTAAACTGAAAGGACAAGTTTTG
GTGACTGCGCTCCTCCAAGCCAGTTACCTCGGTTCAAAGAGTTGGTAGCTCAGAGAACCTTCGAAA
AACCGCCCTGCAAGGCGGTTTTTTCGTTTTCAGAGCAAGAGATTACGCGCAGACCAAAACGATCTC
AAGAAGATCATCTTATTAATCAGATAAAATATTTGCTCATGAGCCCGAAGTGGCGAGCCCGATCTT
CCCCATCGGTGATGTCGGCGATATAGGCGCCAGCAACCGCACCTGTGGCGCCGGTGATGCCGGCC
ACGATGCGTCCGGCGTAGAGGATCTGCTCATGTTTGACAGCTTATCATCGATGCATAATGTGCCTG
TCAAATGGACGAATTAATTAAGTAGGTGTTCCACAGGGTAGCCAGCAGCATCCTGCGATGCAGAT
CCGGAACATAATGGTGCAGGGCGCTGACTTCCGCGTTTCCAGACTTTACGAAACACGGAAACCGA
AGACCATTCATGTTGTTGCTCAGGTCGCAGACGTTTTGCAGCAGCAGTCGCTTCACGTTCGCTCGC
GTATCGGTGATTCATTCTGCTAACCAGTAAGGCAACCCCGCCAGCCTAGCCGGGTCCTCAACGACA
GGAGCACGATCATGCTAGTCATGCCCCGCGCCCACCGGAAGGAGCTGACTGGGTTGAAGGCTCTC
AAGGGCATCGGTCGAGATCCCGGTGCCTAATGAGTGAGCTAACTTACATTAATTGCGTTGCGCTCA
CTGCCCGCTTTCCAGTCGGGAAACCTGTCGTGCCAGCTGCATTAATGAATCGGCCAACGCGCGGGG
AGAGGCGGTTTGCGTATTGGGCGCCAGGGTGGTTTTTCTTTTCACCAGTGAGACGGGCAACAGCTG
ATTGCCCTTCACCGCCTGGCCCTGAGAGAGTTGCAGCAAGCGGTCCACGCTGGTTTGCCCCAGCAG
GCGAAAATCCTGTTTGATGGTGGTTAACGGCGGGATATAACATGAGCTGTCTTCGGTATCGTCGTA
TCCCACTACCGAGATGTCCGCACCAACGCGCAGCCCGGACTCGGTAATGGCGCGCATTGCGCCCA
GCGCCATCTGATCGTTGGCAACCAGCATCGCAGTGGGAACGATGCCCTCATTCAGCATTTGCATGG
TTTGTTGAAAACCGGACATGGCACTCCAGTCGCCTTCCCGTTCCGCTATCGGCTGAATTTGATTGCG
AGTGAGATATTTATGCCAGCCAGCCAGACGCAGACGCGCCGAGACAGAACTTAATGGGCCCGCTA
ACAGCGCGATTTGCTGGTGACCCAATGCGACCAGATGCTCCACGCCCAGTCGCGTACCGTCTTCAT
GGGAGAAAATAATACTGTTGATGGGTGTCTGGTCAGAGACATCAAGAAATAACGCCGGAACATTA
GTGCAGGCAGCTTCCACAGCAATGGCATCCTGGTCATCCAGCGGATAGTTAATGATCAGCCCACTG
ACGCGTTGCGCGAGAAGATTGTGCACCGCCGCTTTACAGGCTTCGACGCCGCTTCGTTCTACCATC
GACACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTGTT
GTGCCACGCGGTTGGGAATGTAATTCAGCTCCGCCATCGCCGCTTCCACTTTTTCCCGCGTTTTCGC
AGAAACGTGGCTGGCCTGGTTCACCACGCGGGAAACGGTCTGATAAGAGACACCGGCATACTCTG
CGACATCGTATAACGTTACTGGTTTCACATTCACCACCCTGAATTGACTCTCTTCCGGGCGCTATCA
TGCCATACCGCGAAAGGTTTTGCGCCATTCGATGGTGTCCGGGATCTCGACGCTCTCCCTTATGCG
ACTCCTGCATTAGGAAGCAGCCCAGTAGTAGGTTGAGGCCGTTGAGCACCGCCGCCGCAAGGAAT
GGTGCATGCAAGGAGATGGCGCCCAACAGTCCCCCGGCCACGGGGCCTGCCACCATACCCACGCC
GAAACAAGCGCTCATGAGCCCGAAGTGGCGAGCCCGATCTTCCCCATCGGTGATGTCGGCGATAT
AGGCGCCAGCAACCGCACCTGTGGCGCCGGTGATGCCGGCCACGATGCGTCCTCGAGTTTATGGCT
AGCTCAGTCCTAGGTACAATGCTAGCAATTGTGAGCGGATAACAAGGCTAGCGAATTCGAGCTCC
CTCTAGAAATAATTTTGTTTAACTTTAAGAAGGAGATATACCATGGGCAGCAGCCATCATCATCAT
CATCACATGTCTTCTGAAACCGGTCCGGTTGCGGTTGACCCGACCCTGCGTCGTCGTATCGAACCG
CACGAATTCGAAGTTTTCTTCGACCCGCGTGAACTGCGTAAAGAAACCTGCCTGCTGTACGAAATC
AACTGGGGTGGTCGTCACTCTATCTGGCGTCACACCTCTCAGAACACCAACAAACACGTTGAAGTT
AACTTCATCGAAAAATTCACCACCGAACGTTACTTCTGCCCGAACACCCGTTGCTCTATCACCTGG
TTCCTGTCTTGGTCTCCGTGCGGTGAATGCTCTCGTGCGATCACCGAATTCCTGTCTCGTTACCCGC
ACGTTACCCTGTTCATCTACATCGCGCGTCTGTACCACCACGCGGACCCGCGTAACCGTCAGGGTC
TGCGTGACCTGATCTCTTCTGGTGTTACCATCCAGATCATGACCGAACAGGAATCTGGTTACTGCT
GGCGTAACTTCGTTAACTACTCTCCGTCTAACGAAGCGCACTGGCCGCGTTACCCGCACCTGTGGG
TTCGTCTGTACGTTCTGGAACTGTACTGCATCATCCTGGGTCTGCCGCCGTGCCTGAACATCCTGCG
TCGTAAACAGCCGCAGCTGACCTTCTTCACCATCGCGCTGCAGTCTTGCCACTACCAGCGTCTGCC
GCCGCACATCCTGTGGGCGACCGGTCTGAAAGGCGGTAGCGGAGGGAGTGGCGGTAGCGGAGGG
AGTGGGAGCTCAAGAGGATACCATATGAACACGATTAACATCGCTAAGAACGACTTCTCTGACAT
CGAACTGGCTGCTATCCCGTTCAACACTCTGGCTGACCATTACGGTGAGCGTTTAGCTCGCGAACA
GTTGGCCCTTGAGCATGAGTCTTACGAGATGGGTGAAGCACGCTTCCGCAAGATGTTTGAGCGTCA
ACTTAAAGCTGGTGAGGTTGCGGATAACGCTGCCGCCAAGCCTCTCATCACTACCCTACTCCCTAA
GATGATTGCACGCATCAACGACTGGTTTGAGGAAGTGAAAGCTAAGCGCGGCAAGCGCCCGACAG
CCTTCCAGTTCCTGCAAGAAATCAAGCCGGAAGCCGTAGCGTACATCACCATTAAGACCACTCTGG
CTTGCCTAACCAGTGCTGACAATACAACCGTTCAGGCTGTAGCAAGCGCAATCGGTCGGGCCATTG
AGGACGAGGCTCGCTTCGGTCGTATCCGTGACCTTGAAGCTAAGCACTTCAAGAAAAACGTTGAG
GAACAACTCAACAAGCGCGTAGGGCACGTCTACAAGAAAGCATTTATGCAAGTTGTCGAGGCTGA
CATGCTCTCTAAGGGTCTACTCGGTGGCGAGGCGTGGTCTTCGTGGCATAAGGAAGACTCTATTCA
TGTAGGAGTACGCTGCATCGAGATGCTCATTGAGTCAACCGGAATGGTTAGCTTACACCGCCAAA
ATGCTGGCGTAGTAGGTCAAGACTCTGAGACTATCGAACTCGCACCTGAATACGCTGAGGCTATCG
CAACCCGTGCAGGTGCGCTGGCTGGCATCTCTCCGATGTTCCAACCTTGCGTAGTTCCTCCTAAGC
CGTGGACTGGCATTACTGGTGGTGGCTATTGGGCTAACGGTCGTCGTCCTCTGGCGCTGGTGCGTA
CTCACAGTAAGAAAGCACTGATGCGCTACGAAGACGTTTACATGCCTGAGGTGTACAAAGCGATT
AACATTGCGCAAAACACCGCATGGAAAATCAACAAGAAAGTCCTAGCGGTCGCCAACGTAATCAC
CAAGTGGAAGCATTGTCCGGTCGAGGACATCCCTGCGATTGAGCGTGAAGAACTCCCGATGAAAC
CGGAAGACATCGACATGAATCCTGAGGCTCTCACCGCGTGGAAACGTGCTGCCGCTGCTGTGTACC
GCAAGGACAAGGCTCGCAAGTCTCGCCGTATCAGCCTTGAGTTCATGCTTGAGCAAGCCAATAAG
TTTGCTAACCATAAGGCCATCTGGTTCCCTTACAACATGGACTGGCGCGGTCGTGTTTACGCTGTGT
CAATGTTCAACCCGCAAGGTAACGATATGACCAAAGGACTGCTTACGCTGGCGAAAGGTAAACCA
ATCGGTAAGGAAGGTTACTACTGGCTGAAAATCCACGGTGCAAACTGTGCGGGTGTCGATAAGGT
TCCGTTCCCTGAGCGCATCAAGTTCATTGAGGAAAACCACGAGAACATCATGGCTTGCGCTAAGTC
TCCACTGGAGAACACTTGGTGGGCTGAGCAAGATTCTCCGTTCTGCTTCCTTGCGTTCTGCTTTGAG
TACGCTGGGGTACAGCACCACGGCCTGAGCTATAACTGCTCCCTTCCGCTGGCGTTTGACGGGTCT
TGCTCTGGCATCCAGCACTTCTCCGCGATGCTCCGAGATGAGGTAGGTGGTCGCGCGGTTAACTTG
CTTCCTAGTGAAACCGTTCAGGACATCTACGGGATTGTTGCTAAGAAAGTCAACGAGATTCTACAA
GCAGACGCAATCAATGGGACCGATAACGAAGTAGTTACCGTGACCGATGAGAACACTGGTGAAAT
CTCTGAGAAAGTCAAGCTGGGCACTAAGGCACTGGCTGGTCAATGGCTGGCTTACGGTGTTACTCG
CAGTGTGACTAAGCGTTCAGTCATGACGCTGGCTTACGGGTCCAAAGAGTTCGGCTTCCGTCAACA
AGTGCTGGAAGATACCATTCAGCCAGCTATTGATTCCGGCAAGGGTCTGATGTTCACTCAGCCGAA
TCAGGCTGCTGGATACATGGCTAAGCTGATTTGGGAATCTGTGAGCGTGACGGTGGTAGCTGCGGT
TGAAGCAATGAACTGGCTTAAGTCTGCTGCTAAGCTGCTGGCTGCTGAGGTCAAAGATAAGAAGA
CTGGAGAGATTCTTCGCAAGCGTTGCGCTGTGCATTGGGTAACTCCTGATGGTTTCCCTGTGTGGC
AGGAATACAAGAAGCCTATTCAGACGCGCTTGAACCTGATGTTCCTCGGTCAGTTCCGCTTACAGC
CTACCATTAACACCAACAAAGATAGCGAGATTGATGCACACAAACAGGAGTCTGGTATCGCTCCT
AACTTTGTACACAGCCAAGACGGTAGCCACCTTCGTAAGACTGTAGTGTGGGCACACGAGAAGTA
CGGAATCGAATCTTTTGCACTGATTCACGACTCCTTCGGTACCATTCCGGCTGACGCTGCGAACCT
GTTCAAAGCAGTGCGCGAAACTATGGTTGACACATATGAGTCTTGTGATGTACTGGCTGATTTCTA
CGACCAGTTCGCTGACCAGTTGCACGAGTCTCAATTGGACAAAATGCCAGCACTTCCGGCTAAAG
GTAACTTGAACCTCCGTGACATCTTAGAGTCGGACTTCGCGTTCGCGTAATCTAGAGTCGACCTGC
AGGCATGCAAGCTTGGCTGTTTTGGCGGATGAGAGAAGATTTTCAGCCTGATACAGATTAAATCAG
AACGCAGAAGCGGTCTGATAAAACAGAATTTGCCTGGCGGCAGTAGCGCGGTGGTCCCACCTGAC
CCCATGCCGAACTCAGAAGTGAAACGCCGTAGCGCCGATGGTAGTGTGGGGTCTCCCCATGCGAG
AGTAGGGAACTGCCAGGCATCAAATAAAACGAAAGGCTCAGTCGAAAGACTGGGCCTTTCGTTTT
ATCTGTTGTTTGTCGGTGAACGCTCTCCTGAGTAGGACAAATCCGCCGGGAGCGGATTTGAACGTT
GCGAAGCAACGGCCCGGAGGGTGGCGGGCAGGACGCCCGCCATAAACTGCCAGGCATCAAATTA
AGCAGAAGGCCATCCTGACGGATGGCCTTTTTGCGTTTCTACAAACTCTTTTGTTTATTTTTCTAAA
TACATTCAAATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTGGGTGCAGCAA
ACT
*BBa_J23114 lacO rApo1
(SEQ ID NO: 70)
GGCGAACTACTTACTCTAGCTTCCCGGCAACAATTAATAGACTGGATGGAGGCGGATAAAGTTGC
AGGACCACTTCTGCGCTCGGCCCTTCCGGCTGGCTGGTTTATTGCTGATAAATCTGGAGCCGGTGA
GCGTGGGTCTCGCGGTATCATTGCAGCACTGGGGCCAGATGGTAAGCCCTCCCGTATCGTAGTTAT
CTACACGACGGGGAGTCAGGCAACTATGATGAACGAAATAGACAGATCGCTGAGATAGGTGCCTC
ACTGATTAAGCATTGGTAACTGTCAGACCAAGTTTACTCATATATACTTTAGATTGATTTACGCGCC
CTGTAGCGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGCTTTCCCCGT
CAAGCTCTAAATCGGGGGCTCCCTTTAGGGTTCCGATTTAGTGCTTTACGGCACCTCGACCCCAAA
AAACTTGATTTGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTTGAACAACACTCAACCCTATCT
CGGGCTATTCTTTTGATTTATAAGGGATTTTGCCGATTTCGGCCATTGGTTAAAAAATGAGCTGATT
TAACAAAAATTTAACGCGAATTTTAACAAAATATTAACGTTTACAATTTAAAAGGATCTAGGTGAA
GATCCTTTTTGATAATCTCATGACCAAAATCCCTTAACGTGAGTTTTCGTTCCACTGAGCGTCAGAC
CCCGTAGAAAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCCGTAGTTAGGCC
ACCACTTCAAGAACTCTGTAGCACCGCCTACATACCTCGCTCTGCTAATCCTGTTACCAGTCAGGC
ATTTGAGAAGCACACGGTCACACTGCTTCCGGTAGTCAATAAACCGGTAAACCAGCAATAGACAT
AAGCGGCTATTTAACGACCCTGCCCTGAACCGACGACCGGGTCGAATTTGCTTTCGAATTTCTGCC
ATTCATCCGCTTATTATCACTTATTCAGGCGTAGCACCAGGCGTTTAAGGGCACCAATAACTGCCT
TAAAAAAATTACGCCCCGCCCTGCCACTCATCGCAGTACTGTTGTAATTCATTAAGCATTCTGCCG
ACATGGAAGCCATCACAGACGGCATGATGAACCTGAATCGCCAGCGGCATCAGCACCTTGTCGCC
TTGCGTATAATATTTGCCCATGGTGAAAACGGGGGCGAAGAAGTTGTCCATATTGGCCACGTTTAA
ATCAAAACTGGTGAAACTCACCCAGGGATTGGCTGAGACGAAAAACATATTCTCAATAAACCCTT
TAGGGAAATAGGCCAGGTTTTCACCGTAACACGCCACATCTTGCGAATATATGTGTAGAAACTGCC
GGAAATCGTCGTGGTATTCACTCCAGAGCGATGAAAACGTTTCAGTTTGCTCATGGAAAACGGTGT
AACAAGGGTGAACACTATCCCATATCACCAGCTCACCGTCTTTCATTGCCATACGGAATTCCGGAT
GAGCATTCATCAGGCGGGCAAGAATGTGAATAAAGGCCGGATAAAACTTGTGCTTATTTTTCTTTA
CGGTCTTTAAAAAGGCCGTAATATCCAGCTGAACGGTCTGGTTATAGGTACATTGAGCAACTGACT
GAAATGCCTCAAAATGTTCTTTACGATGCCATTGGGATATATCAACGGTGGTATATCCAGTGATTT
TTTTCTCCATTTTAGCTTCCTTAGCTCCTGAAAATCTCGATAACTCAAAAAATACGCCCGGTAGTGA
TCTTATTTCATTATGGTGAAAGTTGGAACCTCTTACGTGCCGATCAACGTCTCATTTTCGCCAAAAG
TTGGCCCAGGGCTTCCCGGTATCAACAGGGACACCAGGATTTATTTATTCTGCGAAGTGATCTTCC
GTCACAGGTATTTATTCGGCGCAAAGTGCGTCGGGTGATGCTGCCAACTTACTGATTTAGTGTATG
ATGGTGTTTTTGAGGTGCTCCAGTGGCTTCTGTTTCTATCAGCTGTCCCTCCTGTTCAGCTACTGAC
GGGGTGGTGCGTAACGGCAAAAGCACCGCCGGACATCAGCGCTAGCGGAGTGTATACTGGCTTAC
TATGTTGGCACTGATGAGGGTGTCAGTGAAGTGCTTCATGTGGCAGGAGAAAAAAGGCTGCACCG
GTGCGTCAGCAGAATATGTGATACAGGATATATTCCGCTTCCTCGCTCACTGACTCGCTACGCTCG
GTCGTTCGACTGCGGCGAGCGGAAATGGCTTACGAACGGGGCGGAGATTTCCTGGAAGATGCCAG
GAAGATACTTAACAGGGAAGTGAGAGGGCCGCGGCAAAGCCGTTTTTCCATAGGCTCCGCCCCCC
TGACAAGCATCACGAAATCTGACGCTCAAATCAGTGGTGGCGAAACCCGACAGGACTATAAAGAT
ACCAGGCGTTTCCCCCTGGCGGCTCCCTCGTGCGCTCTCCTGTTCCTGCCTTTCGGTTTACCGGTGT
CATTCCGCTGTTATGGCCGCGTTTGTCTCATTCCACGCCTGACACTCAGTTCCGGGTAGGCAGTTCG
CTCCAAGCTGGACTGTATGCACGAACCCCCCGTTCAGTCCGACCGCTGCGCCTTATCCGGTAACTA
TCGTCTTGAGTCCAACCCGGAAAGACATGCAAAAGCACCACTGGCAGCAGCCACTGGTAATTGAT
TTAGAGGAGTTAGTCTTGAAGTCATGCGCCGGTTAAGGCTAAACTGAAAGGACAAGTTTTGGTGA
CTGCGCTCCTCCAAGCCAGTTACCTCGGTTCAAAGAGTTGGTAGCTCAGAGAACCTTCGAAAAACC
GCCCTGCAAGGCGGTTTTTTCGTTTTCAGAGCAAGAGATTACGCGCAGACCAAAACGATCTCAAGA
AGATCATCTTATTAATCAGATAAAATATTTGCTCATGAGCCCGAAGTGGCGAGCCCGATCTTCCCC
ATCGGTGATGTCGGCGATATAGGCGCCAGCAACCGCACCTGTGGCGCCGGTGATGCCGGCCACGA
TGCGTCCGGCGTAGAGGATCTGCTCATGTTTGACAGCTTATCATCGATGCATAATGTGCCTGTCAA
ATGGACGAATTAATTAAGTAGGTGTTCCACAGGGTAGCCAGCAGCATCCTGCGATGCAGATCCGG
AACATAATGGTGCAGGGCGCTGACTTCCGCGTTTCCAGACTTTACGAAACACGGAAACCGAAGAC
CATTCATGTTGTTGCTCAGGTCGCAGACGTTTTGCAGCAGCAGTCGCTTCACGTTCGCTCGCGTATC
GGTGATTCATTCTGCTAACCAGTAAGGCAACCCCGCCAGCCTAGCCGGGTCCTCAACGACAGGAG
CACGATCATGCTAGTCATGCCCCGCGCCCACCGGAAGGAGCTGACTGGGTTGAAGGCTCTCAAGG
GCATCGGTCGAGATCCCGGTGCCTAATGAGTGAGCTAACTTACATTAATTGCGTTGCGCTCACTGC
CCGCTTTCCAGTCGGGAAACCTGTCGTGCCAGCTGCATTAATGAATCGGCCAACGCGCGGGGAGA
GGCGGTTTGCGTATTGGGCGCCAGGGTGGTTTTTCTTTTCACCAGTGAGACGGGCAACAGCTGATT
GCCCTTCACCGCCTGGCCCTGAGAGAGTTGCAGCAAGCGGTCCACGCTGGTTTGCCCCAGCAGGCG
AAAATCCTGTTTGATGGTGGTTAACGGCGGGATATAACATGAGCTGTCTTCGGTATCGTCGTATCC
CACTACCGAGATGTCCGCACCAACGCGCAGCCCGGACTCGGTAATGGCGCGCATTGCGCCCAGCG
CCATCTGATCGTTGGCAACCAGCATCGCAGTGGGAACGATGCCCTCATTCAGCATTTGCATGGTTT
GTTGAAAACCGGACATGGCACTCCAGTCGCCTTCCCGTTCCGCTATCGGCTGAATTTGATTGCGAG
TGAGATATTTATGCCAGCCAGCCAGACGCAGACGCGCCGAGACAGAACTTAATGGGCCCGCTAAC
AGCGCGATTTGCTGGTGACCCAATGCGACCAGATGCTCCACGCCCAGTCGCGTACCGTCTTCATGG
GAGAAAATAATACTGTTGATGGGTGTCTGGTCAGAGACATCAAGAAATAACGCCGGAACATTAGT
GCAGGCAGCTTCCACAGCAATGGCATCCTGGTCATCCAGCGGATAGTTAATGATCAGCCCACTGAC
GCGTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGT
GCCACGCGGTTGGGAATGTAATTCAGCTCCGCCATCGCCGCTTCCACTTTTTCCCGCGTTTTCGCAG
AAACGTGGCTGGCCTGGTTCACCACGCGGGAAACGGTCTGATAAGAGACACCGGCATACTCTGCG
ACATCGTATAACGTTACTGGTTTCACATTCACCACCCTGAATTGACTCTCTTCCGGGCGCTATCATG
CCATACCGCGAAAGGTTTTGCGCCATTCGATGGTGTCCGGGATCTCGACGCTCTCCCTTATGCGAC
TCCTGCATTAGGAAGCAGCCCAGTAGTAGGTTGAGGCCGTTGAGCACCGCCGCCGCAAGGAATGG
TGCATGCAAGGAGATGGCGCCCAACAGTCCCCCGGCCACGGGGCCTGCCACCATACCCACGCCGA
AACAAGCGCTCATGAGCCCGAAGTGGCGAGCCCGATCTTCCCCATCGGTGATGTCGGCGATATAG
GCGCCAGCAACCGCACCTGTGGCGCCGGTGATGCCGGCCACGATGCGTCCTCGAGTTTATGGCTAG
CTCAGTCCTAGGTACAATGCTAGCAATTGTGAGCGGATAACAAGGCTAGCGAATTCGAGCTCCCTC
TAGAAATAATTTTGTTTAACTTTAAGAAGGAGATATACCATGGGCAGCAGCCATCATCATCATCAT
CACATGTCTTCTGAAACCGGTCCGGTTGCGGTTGACCCGACCCTGCGTCGTCGTATCGAACCGCAC
GAATTCGAAGTTTTCTTCGACCCGCGTGAACTGCGTAAAGAAACCTGCCTGCTGTACGAAATCAAC
TGGGGTGGTCGTCACTCTATCTGGCGTCACACCTCTCAGAACACCAACAAACACGTTGAAGTTAAC
TTCATCGAAAAATTCACCACCGAACGTTACTTCTGCCCGAACACCCGTTGCTCTATCACCTGGTTCC
TGTCTTGGTCTCCGTGCGGTGAATGCTCTCGTGCGATCACCGAATTCCTGTCTCGTTACCCGCACGT
TACCCTGTTCATCTACATCGCGCGTCTGTACCACCACGCGGACCCGCGTAACCGTCAGGGTCTGCG
TGACCTGATCTCTTCTGGTGTTACCATCCAGATCATGACCGAACAGGAATCTGGTTACTGCTGGCG
TAACTTCGTTAACTACTCTCCGTCTAACGAAGCGCACTGGCCGCGTTACCCGCACCTGTGGGTTCG
TCTGTACGTTCTGGAACTGTACTGCATCATCCTGGGTCTGCCGCCGTGCCTGAACATCCTGCGTCGT
AAACAGCCGCAGCTGACCTTCTTCACCATCGCGCTGCAGTCTTGCCACTACCAGCGTCTGCCGCCG
CACATCCTGTGGGCGACCGGTCTGAAATAACTCGAGCTGTTTTGGCGGATGAGAGAAGATTTTCAG
CCTGATACAGATTAAATCAGAACGCAGAAGCGGTCTGATAAAACAGAATTTGCCTGGCGGCAGTA
GCGCGGTGGTCCCACCTGACCCCATGCCGAACTCAGAAGTGAAACGCCGTAGCGCCGATGGTAGT
GTGGGGTCTCCCCATGCGAGAGTAGGGAACTGCCAGGCATCAAATAAAACGAAAGGCTCAGTCGA
AAGACTGGGCCTTTCGTTTTATCTGTTGTTTGTCGGTGAACGCTCTCCTGAGTAGGACAAATCCGCC
GGGAGCGGATTTGAACGTTGCGAAGCAACGGCCCGGAGGGTGGCGGGCAGGACGCCCGCCATAA
ACTGCCAGGCATCAAATTAAGCAGAAGGCCATCCTGACGGATGGCCTTTTTGCGTTTCTACAAACT
CTTTTGTTTATTTTTCTAAATACATTCAAATATGTATCCGCTCATGAGACAATAACCCTGATAAATG
CTTCAATAATATTGAAAAAGGAAGAGTATGAGTATTCAACATTTCCGTGTCGCCCTTATTCCCTTTT
TTGCGGCATTTTGCCTTCCTGTTTTTGCTCACCCAGAAACGCTGGTGAAAGTAAAAGATGCTGAAG
ATCAGTTGGGTGCAGCAAACTATTAACT
* T7 promoter + rpsL reporter plasmid
(SEQ ID NO: 80)
ACGAAAGGGCCTCGTGATACGCCTATTTTTATAGGTTAATGTCATGATAATAATGGTTTCTTAGAC
GTCAGGTGGCACTTTTCGGGGAAATGTGCCCGGTTAACGTGCCGGCACGGCCTGGGTAACCAGGT
ATTTTGTCCACATAACCGTGCGCAAAATGTTGTGGATAAGCAGGACACAGCAGCAATCCACAGCA
GGCATACAACCGCACACCGAGGTTACTCCGTTCTACAGGTTACGACGACATGTCAATACTTGCCCT
TGACAGGCATTGATGGAATCGTAGTCTCACGCTGATAGTCTGATCGACAATACAAGTGGGACCGT
GGTCCCAGACCGATAATCAGACCGACAACACGAGTGGGATCGTGGTCCCAGACTAATAATCAGAC
CGACGATACGAGTGGGACCGTGGTCCCAGACTAATAATCAGACCGACGATACGAGTGGGACCGTG
GTTCCAGACTAATAATCAGACCGACGATACGAGTGGGACCGTGGTCCCAGACTAATAATCAGACC
GACGATACGAGTGGGACCATGGTCCCAGACTAATAATCAGACCGACGATACGAGTGGGACCGTGG
TCCCAGTCTGATTATCAGACCGACGATACGAGTGGGACCGTGGTCCCAGACTAATAATCAGACCG
ACGATACGAGTGGGACCGTGGTCCCAGACTAATAATCAGACCGACGATACGAGTGGGACCGTGGT
CCCAGTCTGATTATCAGACCGACGATACAAGTGGAACAGTGGGCCCAGAGAGAATATTCAGGCCA
GTTATGCTTTCTGGCCTGTAACAAAGGACATTAAGTAAAGACAGATAAACGTAGACTAAAACGTG
GTCGCATCAGGGTGCTGGCTTTTCAAGTTCCTTAAGAATGGCCTCAATTTTCTCTATACACTCAGTT
GGAACACGAGACCTGTCCAGGTTAAGCACCATTTTATCGCCCTTATACAATACTGTCGCTCCAGGA
GCAAACTGATGTCGTGAGCTTAAACTAGTTCTTGATGCAGATGACGTTTTAAGCACAGAAGTTAAA
AGAGTGATAACTTCTTCAGCTTCAAATATCACCCCAGCTTTTTTCTGCTCATGAAGGTTAGATGCCT
GCTGCTTAAGTAATTCCTCTTTATCTGTAAAGGCTTTTTGAAGTGCATCACCTGACCGGGCAGATA
GTTCACCGGGGTGAGAAAAAAGAGCAACAACTGATTTAGGCAATTTGGCGGTGTTGATACAGCGG
GTAATAATCTTACGTGAAATATTTTCCGCATCAGCCAGCGCAGAAATATTTCCAGCAAATTCATTC
TGCAATCGGCTTGCATAACGCTGACCACGTTCATAAGCACTTGTTGGGCGATAATCGTTACCCAAT
CTGGATAATGCAGCCATCTGCTCATCATCCAGCTCGCCAACCAGAACACGATAATCACTTTCGGTA
AGTGCAGCAGCTTTACGACGGCGACTCCCATCGGCAATTTCTATGACACCAGATACTCTTCGACCG
AACGCCGGTGTCTGTTGACCAGTCAGTAGAAAAGAAGGGATGAGATCATCCAGTGCGTCCTCAGT
AAGCAGCTCCTGGTCACGTTCATTACCTGACCATACCCGAGAGGTCTTCTCAACACTATCACCCCG
GAGCACTTCAAGAGTAAACTTCACATCCCGACCACATACAGGCAAAGTAATGGCATTACCGCGAG
CCATTACTCCTACGCGCGCAATTAACGAATCCACCATCGGGGCAGCTGGTGTCGATAACGAAGTAT
CTTCAACCGGTTGAGTATTGAGCGTATGTTTTGGAATAACAGGCGCACGCTTCATTATCTAATCTCC
CAGCGTGGTTTAATCAGACGATCGAAAATTTCATTGCAGACAGGTTCCCAAATAGAAAGAGCATTT
CTCCAGGCACCAGTTGAAGAGCGTTGATCAATGGCCTGTTCAAAAACAGTTCTCATCCGGATCTGA
CCTTTACCAACTTCATCCGTTTCACGTACAACATTTTTTAGAACCATGCTTCCCCAGGCATCCCGAA
TTTGCTCCTCCATCCACGGGGACTGAGAGCCATTACTATTGCTGTATTTGGTAAGCAAAATACGTA
CATCAGGCTCGAACCCTTTAAGATCAACGTTCTTGAGCAGATCACGAAGCATATCGAAAAACTGC
AGTGCGGAGGTGTAGTCAAACAACTCAGCAGGCGTGGGAACAATCAGCACATCAGCAGCACATAC
GACATTAATCGTGCCGATACCCAGGTTAGGCGCGCTGTCAATAACTATGACATCATAGTCATGAGC
AACAGTTTCAATGGCCAGTCGGAGCATCAGGTGTGGATCGGTGGGCAGTTTACCTTCATCAAATTT
GCCCATTAACTCAGTTTCAATACGGTGCAGAGCCAGACAGGAAGGAATAATGTCAAGCCCCGGCC
AGCAAGTGGGCTTTATTGCATAAGTGACATCGTCCTTTTCCCCAAGATAGAAAGGCAGGAGAGTGT
CTTCTGCATGAATATGAAGATCTGGTACCCATCCGTGATACATTGAGGCTGTTCCCTGGGGGTCGT
TACCTTCCACGAGCAAAACACGTAGCCCCTTCAGAGCCAGATCCTGAGCAAGATGAACAGAAACT
GAGGTTTTGTAAACGCCACCTTTATGGGCAGCAACCCCGATCACCGGTGGAAATACGTCTTCAGCA
CGTCGCAATCGCGTACCAAACACATCACGCATATGATTAATTTGTTCAATTGTATAACCAACACGT
TGCTCAACCCGTCCTCGAATTTCCATATCCGGGTGCGGTAGTCGCCCTGCTTTCTCGGCATCTCTGA
TAGCCTGAGAAGAAACCCCAACTAAATCCGCTGCTTCACCTATTCTCCAGCGCCGGGTTATTTTCC
TCGCTTCCGGGCTGTCATCATTAAACTGTGCAATGGCGATAGCCTTCGTCATTTCATGACCAGCGTT
TATGCACTGGTTAAGTGTTTCCATGAGTTTCATTCTGAACATCCTTTAATCATTGCTTTGCGTTTTTT
TATTAAATCTTGCAATTTACTGCAAAGCAACAACAAAATCGCAAAGTCATCAAAAAACCGCAAAG
TTGTTTAAAATAAGAGCAACACTACAAAAGGAGATAAGAAGAGCACATACCTCAGTCACTTATTA
TCACTAGCGCTCGCCGCAGCCGTGTAACCGAGCATAGCGAGCGAACTGGCGAGGAAGCAAAGAA
GAACTGTTCTGTCAGATAGCTCTTACGCTCAGCGCAAGAAGAAATATCCACCGTGGGAAAAACTC
CAGGTAGAGGTACACACGCGGATAGCCAATTCAGAGTAATAAACTGTGATAATCAACCCTCATCA
ATGATGACGAACTAACCCCCGATATCAGGTCACATGACGAAGGGAAAGAGAAGGAAATCAACTGT
GACAAACTGCCCTCAAATTTGGCTTCCTTAAAAATTACAGTTCAAAAAGTATGAGAAAATCCATGC
AGGCTGAAGGAAACAGCAAAACTGTGACAAATTACCCTCAGTAGGTCAGAACAAATGTGACGAAC
CACCCTCAAATCTGTGACAGATAACCCTCAGACTATCCTGTCGTCATGGAAGTGATATCGCGGAAG
GAAAATACGATATGAGTCGTCTGGCGGCCTTTCTTTTTCTCAATGTATGAGAGGCGCATTGGAGTT
CTGCTGTTGATCTCATTAACACAGACCTGCAGGAAGCGGCGGCGGAAGTCAGGCATACGCTGGTA
ACTTTGAGGCAGCTGGTAACGCTCTATGATCCAGTCGATTTTCAGAGAGACGATGCCTGAGCCATC
CGGCTTACGATACTGACACAGGGATTCGTATAAACGCATGGCATACGGATTGGTGATTTCTTTTGT
TTCACTAAGCCGAAACTGCGTAAACCGGTTCTGTAACCCGATAAAGAAGGGAATGAGATATGGGT
TGATATGTACACTGTAAAGCCCTCTGGATGGACTGTGCGCACGTTTGATAAACCAAGGAAAAGATT
CATAGCCTTTTTCATCGCCGGCATCCTCTTCAGGGCGATAAAAAACCACTTCCTTCCCCGCGAAAC
TCTTCAATGCCTGCCGTATATCCTTACTGGCTTCCGCAGAGGTCAATCCGAATATTTCAGCATATTT
AGCAACATGGATCTCGCAGATACCGTCATGTTCCTGTAGGGTGCCATCAGATTTTCTGATCTGGTC
AACGAACAGATACAGCATACGTTTTTGATCCCGGGAGAGACTATATGCCGCCTCAGTGAGGTCGTT
TGACTGGACGATTCGCGGGCTATTTTTACGTTTCTTGTGATTGATAACCGCTGTTTCCGCCATGACA
GATCCATGTGAAGTGTGACAAGTTTTTAGATTGTCACACTAAATAAAAAAGAGTCAATAAGCAGG
GATAACTTTGTGAAAAAACAGCTTCTTCTGAGGGCAATTTGTCACAGGGTTAAGGGCAATTTGTCA
CAGACAGGACTGTCATTTGAGGGTGATTTGTCACACTGAAAGGGCAATTTGTCACAACACCTTCTC
TAGAACCAGCATGGATAAAGGCCTACAAGGCGCTCTAAAAAAGAAGATCTAAAAACTATAAAAA
AAATAATTATAAAAATATCCCCGTGGATAAGTGGATAACCCCAAGGGAAGTTTTTTCAGGCATCGT
GTGTAAGCAGAATATATAAGTGCTGTTCCCTGGTGCTTCCTCGCTCACTCGAGCTACGGGGTCTGA
CGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATA
GTTGCCTGACTGCCCGTCGTGTAGATAACTACGATACGGGAGGGCTTACCATCTGGCCCCAGTGCT
GCAATGATACCGCGGGACCCACGCTCACCGGCTCCAGATTTATCAGCAATAAACCAGCCAGCCGG
AAGGGCCGAGCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCAAGGCGAGT
TACATGATCCCCCATGTTGTGAAAAAAAGCGGTTAGCTCCTTCGGTCCTCCGATCGTTGTCAGAAG
TAAGTTGGCCGCAGTGTTATCACTCATGCTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAA
AGTGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAAGT
GCCACCTGGCGGCCGCCTTTCAGCAAAAAACCCCGCGAGACCCCCGAAGAGGCCCCGCGGGGTTA
TGCTAGGTCGACGGAGCTCGAATTCTAATACGACTCACTATAGGGAGACCTTGACAATTAATCATC
GGCTCGTATAATGCCAGAAGCTTAGCAAAAGCTAAAACCAGGAGCTATTTAATGGCAACAGTTAA
CCAGCTGGTACGCAAACCACGTGCTCGCAAAGTTGCGAAAAGCAACGTGCCTGCGCTGGAAGCAT
GCCCGCAAAAACGTGGCGTATGTACTCGTGTATATACTACCACTCCTAAAAAACCGAACTCCGCGC
TGCGTAAAGTATGCCGTGTTCGTCTGACTAACGGTTTCGAAGTGACTTCCTACATCGGTGGTGAAG
GTCACAACCTGCAGGAGCACTCCGTGATCCTGATCCGTGGCGGTCGTGTTAAAGACCTCCCGGGTG
TTCGTTACCACACCGTACGTGGTGCGCTTGACTGCTCCGGCGTTAAAGACCGTAAGCAGGCTCGTT
CCAAGTATGGCGTGAAGCGTCCTAAGGCTTAATGGTTTAATTAACGCAGCCTGAATGGCGAATAG
AAGTTTAAACGCTAGACCTAGCATAACCCCGCGGGGCCTCTTCGGGGGTCTCGCGGGGTTTTTTGC
TGAAAGGCTAGACCTAGCATAACCCCGCGGGGCCTCTTCGGGGGTCTCGCGGGGTTTTTTGCTGAA
AGGCTAGACCTAGCATAACCCCGCGGGGCCTCTTCGGGGGTCTCGCGGGGTTTTTTGCTGAAAGGC
TAGACCTAGCATAACCCCGCGGGGCCTCTTCGGGGGTCTCGCGGGGTTTTTTGCTGAAAGGCTAGA
CCTAGCATAACCCCGCGGGGCCTCTTCGGGGGTCTCGCGGGGTTTTTTGCTGAAAGGCTAGGAAGT
TTAAACGCTAGACCTAGCATAACCCCGCGGGGCCTCTTCGGGGGTCTCGCGGGGTTTTTTGCTGAA
AGGCTAGACCTAGCATAACCCCGCGGGGCCTCTTCGGGGGTCTCGCGGGGTTTTTTGCTGAAAGGC
TAGACCTAGCATAACCCCGCGGGGCCTCTTCGGGGGTCTCGCGGGGTTTTTTGCTGAAAGGCTAGA
CCTAGCATAACCCCGCGGGGCCTCTTCGGGGGTCTCGCGGGGTTTTTTGCTGAAAGGCTAGACCTA
GCATAACCCCGCGGGGCCTCTTCGGGGGTCTCGCGGGGTTTTTTGCTGAAAGGCTAGGATATATTG
ATCCTTGACAGCTTATCATCGATAAGCTTTAATGCGGTAGTTTATCACAGTTGCTAACGCAGTCAG
GCACCGTGTACGAATAGTTCGACAAAGATCGCATTGGTAATTACGTTACTCGATGCCATGGGGATT
GGCCTTATCATGCCAGTCTTGCCAACGTTATTACGTGAATTTATTGCTTCGGAAGATATCGCTAACC
ACTTTGGCGTATTGCTTGCACTTTATGCGTTAATGCAGGTTATCTTTGCTCCTTGGCTTGGAAAAAT
GTCTGACCGATTTGGTCGGCGCCCAGTGCTGTTGTTGTCATTAATAGGCGCATCGCTGGATTACTTA
TTGCTGGCTTTTTCAAGTGCGCTTTGGATGCTGTATTTAGGCCGTTTGCTTTCAGGGATCACAGGAG
CTACTGGGGCTGTCGCGGCATCGGTCATTGCCGATACCACCTCAGCTTCTCAACGCGTGAAGTGGT
TCGGTTGGTTAGGGGCAAGTTTTGGGCTTGGTTTAATAGCGGGGCCTATTATTGGTGGTTTTGCAG
GAGAGATTTCACCGCATAGTCCCTTTTTTATCGCTGCGTTGCTAAATATTGTCACTTTCCTTGTGGT
TATGTTTTGGTTCCGTGAAACCAAAAATACACGTGATAATACAGATACCGAAGTAGGGGTTGAGA
CGCAATCGAATTCGGTATACATCACTTTATTTAAAACGATGCCCATTTTGTTGATTATTTATTTTTC
AGCGCAATTGATAGGCCAAATTCCCGCAACGGTGTGGGTGCTATTTACCGAAAATCGTTTTGGATG
GAATAGCATGATGGTTGGCTTTTCATTAGCGGGTCTTGGTCTTTTACACTCAGTATTCCAAGCCTTT
GTGGCAGGAAGAATAGCCACTAAATGGGGCGAAAAAACGGCAGTACTGCTCGGATTTATTGCAGA
TAGTAGTGCATTTGCCTTTTTAGCGTTTATATCTGAAGGTTGGTTAGTTTTCCCTGTTTTAATTTTAT
TGGCTGGTGGTGGGATCGCTTTACCTGCATTACAGGGAGTGATGTCTATCCAAACAAAGAGTCATC
AGCAAGGTGCTTTACAGGGATTATTGGTGAGCCTTACCAATGCAACCGGTGTTATTGGCCCATTAC
TGTTTGCTGTTATTTATAATCATTCACTACCAATTTGGGATGGCTGGATTTGGATTATTGGTTTAGC
GTTTTACTGTATTATTATCCTGCTATCGATGACCTTCATGTTAACCCCTCAAGCTCAGGGGAGTAAA
CAGGAGACAAGTGCTTAGTGATCCAATTCTTGAAG
* folA + T7 promoter plasmid
(SEQ ID NO: 81)
ACGAAAGGGCCTCGTGATACGCCTATTTTTATAGGTTAATGTCATGATAATAATGGTTTCTTAGAC
GTCAGGTGGCACTTTTCGGGGAAATGTGCCCGGTTAACGTGCCGGCACGGCCTGGGTAACCAGGT
ATTTTGTCCACATAACCGTGCGCAAAATGTTGTGGATAAGCAGGACACAGCAGCAATCCACAGCA
GGCATACAACCGCACACCGAGGTTACTCCGTTCTACAGGTTACGACGACATGTCAATACTTGCCCT
TGACAGGCATTGATGGAATCGTAGTCTCACGCTGATAGTCTGATCGACAATACAAGTGGGACCGT
GGTCCCAGACCGATAATCAGACCGACAACACGAGTGGGATCGTGGTCCCAGACTAATAATCAGAC
CGACGATACGAGTGGGACCGTGGTCCCAGACTAATAATCAGACCGACGATACGAGTGGGACCGTG
GTTCCAGACTAATAATCAGACCGACGATACGAGTGGGACCGTGGTCCCAGACTAATAATCAGACC
GACGATACGAGTGGGACCATGGTCCCAGACTAATAATCAGACCGACGATACGAGTGGGACCGTGG
TCCCAGTCTGATTATCAGACCGACGATACGAGTGGGACCGTGGTCCCAGACTAATAATCAGACCG
ACGATACGAGTGGGACCGTGGTCCCAGACTAATAATCAGACCGACGATACGAGTGGGACCGTGGT
CCCAGTCTGATTATCAGACCGACGATACAAGTGGAACAGTGGGCCCAGAGAGAATATTCAGGCCA
GTTATGCTTTCTGGCCTGTAACAAAGGACATTAAGTAAAGACAGATAAACGTAGACTAAAACGTG
GTCGCATCAGGGTGCTGGCTTTTCAAGTTCCTTAAGAATGGCCTCAATTTTCTCTATACACTCAGTT
GGAACACGAGACCTGTCCAGGTTAAGCACCATTTTATCGCCCTTATACAATACTGTCGCTCCAGGA
GCAAACTGATGTCGTGAGCTTAAACTAGTTCTTGATGCAGATGACGTTTTAAGCACAGAAGTTAAA
AGAGTGATAACTTCTTCAGCTTCAAATATCACCCCAGCTTTTTTCTGCTCATGAAGGTTAGATGCCT
GCTGCTTAAGTAATTCCTCTTTATCTGTAAAGGCTTTTTGAAGTGCATCACCTGACCGGGCAGATA
GTTCACCGGGGTGAGAAAAAAGAGCAACAACTGATTTAGGCAATTTGGCGGTGTTGATACAGCGG
GTAATAATCTTACGTGAAATATTTTCCGCATCAGCCAGCGCAGAAATATTTCCAGCAAATTCATTC
TGCAATCGGCTTGCATAACGCTGACCACGTTCATAAGCACTTGTTGGGCGATAATCGTTACCCAAT
CTGGATAATGCAGCCATCTGCTCATCATCCAGCTCGCCAACCAGAACACGATAATCACTTTCGGTA
AGTGCAGCAGCTTTACGACGGCGACTCCCATCGGCAATTTCTATGACACCAGATACTCTTCGACCG
AACGCCGGTGTCTGTTGACCAGTCAGTAGAAAAGAAGGGATGAGATCATCCAGTGCGTCCTCAGT
AAGCAGCTCCTGGTCACGTTCATTACCTGACCATACCCGAGAGGTCTTCTCAACACTATCACCCCG
GAGCACTTCAAGAGTAAACTTCACATCCCGACCACATACAGGCAAAGTAATGGCATTACCGCGAG
CCATTACTCCTACGCGCGCAATTAACGAATCCACCATCGGGGCAGCTGGTGTCGATAACGAAGTAT
CTTCAACCGGTTGAGTATTGAGCGTATGTTTTGGAATAACAGGCGCACGCTTCATTATCTAATCTCC
CAGCGTGGTTTAATCAGACGATCGAAAATTTCATTGCAGACAGGTTCCCAAATAGAAAGAGCATTT
CTCCAGGCACCAGTTGAAGAGCGTTGATCAATGGCCTGTTCAAAAACAGTTCTCATCCGGATCTGA
CCTTTACCAACTTCATCCGTTTCACGTACAACATTTTTTAGAACCATGCTTCCCCAGGCATCCCGAA
TTTGCTCCTCCATCCACGGGGACTGAGAGCCATTACTATTGCTGTATTTGGTAAGCAAAATACGTA
CATCAGGCTCGAACCCTTTAAGATCAACGTTCTTGAGCAGATCACGAAGCATATCGAAAAACTGC
AGTGCGGAGGTGTAGTCAAACAACTCAGCAGGCGTGGGAACAATCAGCACATCAGCAGCACATAC
GACATTAATCGTGCCGATACCCAGGTTAGGCGCGCTGTCAATAACTATGACATCATAGTCATGAGC
AACAGTTTCAATGGCCAGTCGGAGCATCAGGTGTGGATCGGTGGGCAGTTTACCTTCATCAAATTT
GCCCATTAACTCAGTTTCAATACGGTGCAGAGCCAGACAGGAAGGAATAATGTCAAGCCCCGGCC
AGCAAGTGGGCTTTATTGCATAAGTGACATCGTCCTTTTCCCCAAGATAGAAAGGCAGGAGAGTGT
CTTCTGCATGAATATGAAGATCTGGTACCCATCCGTGATACATTGAGGCTGTTCCCTGGGGGTCGT
TACCTTCCACGAGCAAAACACGTAGCCCCTTCAGAGCCAGATCCTGAGCAAGATGAACAGAAACT
GAGGTTTTGTAAACGCCACCTTTATGGGCAGCAACCCCGATCACCGGTGGAAATACGTCTTCAGCA
CGTCGCAATCGCGTACCAAACACATCACGCATATGATTAATTTGTTCAATTGTATAACCAACACGT
TGCTCAACCCGTCCTCGAATTTCCATATCCGGGTGCGGTAGTCGCCCTGCTTTCTCGGCATCTCTGA
TAGCCTGAGAAGAAACCCCAACTAAATCCGCTGCTTCACCTATTCTCCAGCGCCGGGTTATTTTCC
TCGCTTCCGGGCTGTCATCATTAAACTGTGCAATGGCGATAGCCTTCGTCATTTCATGACCAGCGTT
TATGCACTGGTTAAGTGTTTCCATGAGTTTCATTCTGAACATCCTTTAATCATTGCTTTGCGTTTTTT
TATTAAATCTTGCAATTTACTGCAAAGCAACAACAAAATCGCAAAGTCATCAAAAAACCGCAAAG
TTGTTTAAAATAAGAGCAACACTACAAAAGGAGATAAGAAGAGCACATACCTCAGTCACTTATTA
TCACTAGCGCTCGCCGCAGCCGTGTAACCGAGCATAGCGAGCGAACTGGCGAGGAAGCAAAGAA
GAACTGTTCTGTCAGATAGCTCTTACGCTCAGCGCAAGAAGAAATATCCACCGTGGGAAAAACTC
CAGGTAGAGGTACACACGCGGATAGCCAATTCAGAGTAATAAACTGTGATAATCAACCCTCATCA
ATGATGACGAACTAACCCCCGATATCAGGTCACATGACGAAGGGAAAGAGAAGGAAATCAACTGT
GACAAACTGCCCTCAAATTTGGCTTCCTTAAAAATTACAGTTCAAAAAGTATGAGAAAATCCATGC
AGGCTGAAGGAAACAGCAAAACTGTGACAAATTACCCTCAGTAGGTCAGAACAAATGTGACGAAC
CACCCTCAAATCTGTGACAGATAACCCTCAGACTATCCTGTCGTCATGGAAGTGATATCGCGGAAG
GAAAATACGATATGAGTCGTCTGGCGGCCTTTCTTTTTCTCAATGTATGAGAGGCGCATTGGAGTT
CTGCTGTTGATCTCATTAACACAGACCTGCAGGAAGCGGCGGCGGAAGTCAGGCATACGCTGGTA
ACTTTGAGGCAGCTGGTAACGCTCTATGATCCAGTCGATTTTCAGAGAGACGATGCCTGAGCCATC
CGGCTTACGATACTGACACAGGGATTCGTATAAACGCATGGCATACGGATTGGTGATTTCTTTTGT
TTCACTAAGCCGAAACTGCGTAAACCGGTTCTGTAACCCGATAAAGAAGGGAATGAGATATGGGT
TGATATGTACACTGTAAAGCCCTCTGGATGGACTGTGCGCACGTTTGATAAACCAAGGAAAAGATT
CATAGCCTTTTTCATCGCCGGCATCCTCTTCAGGGCGATAAAAAACCACTTCCTTCCCCGCGAAAC
TCTTCAATGCCTGCCGTATATCCTTACTGGCTTCCGCAGAGGTCAATCCGAATATTTCAGCATATTT
AGCAACATGGATCTCGCAGATACCGTCATGTTCCTGTAGGGTGCCATCAGATTTTCTGATCTGGTC
AACGAACAGATACAGCATACGTTTTTGATCCCGGGAGAGACTATATGCCGCCTCAGTGAGGTCGTT
TGACTGGACGATTCGCGGGCTATTTTTACGTTTCTTGTGATTGATAACCGCTGTTTCCGCCATGACA
GATCCATGTGAAGTGTGACAAGTTTTTAGATTGTCACACTAAATAAAAAAGAGTCAATAAGCAGG
GATAACTTTGTGAAAAAACAGCTTCTTCTGAGGGCAATTTGTCACAGGGTTAAGGGCAATTTGTCA
CAGACAGGACTGTCATTTGAGGGTGATTTGTCACACTGAAAGGGCAATTTGTCACAACACCTTCTC
TAGAACCAGCATGGATAAAGGCCTACAAGGCGCTCTAAAAAAGAAGATCTAAAAACTATAAAAA
AAATAATTATAAAAATATCCCCGTGGATAAGTGGATAACCCCAAGGGAAGTTTTTTCAGGCATCGT
GTGTAAGCAGAATATATAAGTGCTGTTCCCTGGTGCTTCCTCGCTCACTCGAGCTACGGGGTCTGA
CGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATA
GTTGCCTGACTGCCCGTCGTGTAGATAACTACGATACGGGAGGGCTTACCATCTGGCCCCAGTGCT
GCAATGATACCGCGGGACCCACGCTCACCGGCTCCAGATTTATCAGCAATAAACCAGCCAGCCGG
AAGGGCCGAGCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCAAGGCGAGT
TACATGATCCCCCATGTTGTGAAAAAAAGCGGTTAGCTCCTTCGGTCCTCCGATCGTTGTCAGAAG
TAAGTTGGCCGCAGTGTTATCACTCATGCTTATGGCAGCACTGCATAATTCTCTTACTGTCATGCCA
TCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAA
AGTGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTTTCT
GGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAAGT
GCCACCTGGCGGCCGCCTTTCAGCAAAAAACCCCGCGAGACCCCCGAAGAGGCCCCGCGGGGTTA
TGCTAGGTCGACGGAGCTCGAATTCTAATACGACTCACTATAGGGGCGTCGCATCCGGCGCTAGCC
GTAAATTCTATACAAAATTACCGCCGCTCCAGAATCTCAAAGCAATAGCTGTGAGAGTTCTGCGCA
TCAGCATCGTGGAATTCGCTGAATACCGATTCCCAGTCATCCGGCTCGTAATCCGGGAAATGGGTG
TCGCCTTCCACTTCTGCGTCGATATGCGTCAGATACAGTTTTTGCGCTTTTGGCAAGAACTGTTCAT
AAACGCGACCGCCGCCAATCACCATGATTTCTGGTACGTCACCACACGCCGCGATGGCTTCATCCA
CCGACTTCACCCACGTTACGCGATCGTCCGTACCCGGTTGACTGCTGAGGATAATATTTTTGCGTCC
TGGCAACGGACGACCGATTGATTCCCAGGTATGGCGGCCCATAATCACGGGTTTATTTAAGGTGTT
GCGTTTAAACCAGGCGAGATCGGCAGGCAGGTTCCACGGCATGGCGTTTTCCATGCCGATAACGC
GATCTACCGCTAACGCCGCAATCAGACTGATCATTGAGATTTCCCGATAAAAAAAATTGTCGCCAC
TATACGTAAAGCGTAAACCGTCGTCGACTGGTGCGAGGATGATGTTGAGGAAAATTTTATATTCTG
CTGGCGAGTCCACGCTCTCTCCCTGGACTCGCCGCATTACAATGAAACAAAAACAAACAGTTAGCT
GTAAAGTGTGATTTACGTCACTCTTTATTAGGATGAGGGTTTCGTTTCCGGTTCATCCTTAATTAAC
GCAGCCTGAATGGCGAATAGAAGTTTAAACGCTAGACCTAGCATAACCCCGCGGGGCCTCTTCGG
GGGTCTCGCGGGGTTTTTTGCTGAAAGGCTAGACCTAGCATAACCCCGCGGGGCCTCTTCGGGGGT
CTCGCGGGGTTTTTTGCTGAAAGGCTAGACCTAGCATAACCCCGCGGGGCCTCTTCGGGGGTCTCG
CGGGGTTTTTTGCTGAAAGGCTAGACCTAGCATAACCCCGCGGGGCCTCTTCGGGGGTCTCGCGGG
GTTTTTTGCTGAAAGGCTAGACCTAGCATAACCCCGCGGGGCCTCTTCGGGGGTCTCGCGGGGTTT
TTTGCTGAAAGGCTAGGAAGTTTAAACGCTAGACCTAGCATAACCCCGCGGGGCCTCTTCGGGGGT
CTCGCGGGGTTTTTTGCTGAAAGGCTAGACCTAGCATAACCCCGCGGGGCCTCTTCGGGGGTCTCG
CGGGGTTTTTTGCTGAAAGGCTAGACCTAGCATAACCCCGCGGGGCCTCTTCGGGGGTCTCGCGGG
GTTTTTTGCTGAAAGGCTAGACCTAGCATAACCCCGCGGGGCCTCTTCGGGGGTCTCGCGGGGTTT
TTTGCTGAAAGGCTAGACCTAGCATAACCCCGCGGGGCCTCTTCGGGGGTCTCGCGGGGTTTTTTG
CTGAAAGGCTAGGATATATTGATCCTTGACAGCTTATCATCGATAAGCTTTAATGCGGTAGTTTAT
CACAGTTGCTAACGCAGTCAGGCACCGTGTACGAATAGTTCGACAAAGATCGCATTGGTAATTAC
GTTACTCGATGCCATGGGGATTGGCCTTATCATGCCAGTCTTGCCAACGTTATTACGTGAATTTATT
GCTTCGGAAGATATCGCTAACCACTTTGGCGTATTGCTTGCACTTTATGCGTTAATGCAGGTTATCT
TTGCTCCTTGGCTTGGAAAAATGTCTGACCGATTTGGTCGGCGCCCAGTGCTGTTGTTGTCATTAAT
AGGCGCATCGCTGGATTACTTATTGCTGGCTTTTTCAAGTGCGCTTTGGATGCTGTATTTAGGCCGT
TTGCTTTCAGGGATCACAGGAGCTACTGGGGCTGTCGCGGCATCGGTCATTGCCGATACCACCTCA
GCTTCTCAACGCGTGAAGTGGTTCGGTTGGTTAGGGGCAAGTTTTGGGCTTGGTTTAATAGCGGGG
CCTATTATTGGTGGTTTTGCAGGAGAGATTTCACCGCATAGTCCCTTTTTTATCGCTGCGTTGCTAA
ATATTGTCACTTTCCTTGTGGTTATGTTTTGGTTCCGTGAAACCAAAAATACACGTGATAATACAGA
TACCGAAGTAGGGGTTGAGACGCAATCGAATTCGGTATACATCACTTTATTTAAAACGATGCCCAT
TTTGTTGATTATTTATTTTTCAGCGCAATTGATAGGCCAAATTCCCGCAACGGTGTGGGTGCTATTT
ACCGAAAATCGTTTTGGATGGAATAGCATGATGGTTGGCTTTTCATTAGCGGGTCTTGGTCTTTTAC
ACTCAGTATTCCAAGCCTTTGTGGCAGGAAGAATAGCCACTAAATGGGGCGAAAAAACGGCAGTA
CTGCTCGGATTTATTGCAGATAGTAGTGCATTTGCCTTTTTAGCGTTTATATCTGAAGGTTGGTTAG
TTTTCCCTGTTTTAATTTTATTGGCTGGTGGTGGGATCGCTTTACCTGCATTACAGGGAGTGATGTC
TATCCAAACAAAGAGTCATCAGCAAGGTGCTTTACAGGGATTATTGGTGAGCCTTACCAATGCAAC
CGGTGTTATTGGCCCATTACTGTTTGCTGTTATTTATAATCATTCACTACCAATTTGGGATGGCTGG
ATTTGGATTATTGGTTTAGCGTTTTACTGTATTATTATCCTGCTATCGATGACCTTCATGTTAACCCC
TCAAGCTCAGGGGAGTAAACAGGAGACAAGTGCTTAGTGATCCAATTCTTGAAG
* C1A
(SEQ ID NO: 82)
CATCATCAATAATATACCTTATTTTGGATTGAAGCCAATATGATAATGAGGGGGTGGAGTTTGTGA
CGTGGCGCGGGGCGTGGGAACGGGGCGGGTGACGTAGTAGTGTGGCGGAAGTGTGATGTTGCAAG
TGTGGCGGAACACATGTAAGCGACGGATGTGGCAAAAGTGACGTTTTTGGTGTGCGCCGGTGTAC
ACAGGAAGTGACAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAATTTTGTGTTACT
CATAGCGCGTAATATTTGTCTAGGGCCGCGGGGACTTTGACCGTTTACGTGGAGACTCGCCCAGGT
GTTTTTCTCAGGTGTTTTCCGCGTTCCGGGTCAAAGTTGGCGTTTTATTATTATAGTCAGTCGAAGC
TTGGATCCGGTACCTCTAGAATTCTCGAGCGGCCGCTAGCGACATCGGATCTCCCGATCCCCTATG
GTGCACTCTCAGTACAATCTGCTCTGATGCCGCATAGTTAAGCCAGTATCTGCTCCCTGCTTGTGTG
TTGGAGGTCGCTGAGTAGTGCGCGAGCAAAATTTAAGCTACAACAAGGCAAGGCTTGACCGACAA
TTGCATGAAGAATCTGCTTAGGGTTAGGCGTTTTGCGCTGCTTCGCGATGTACGGGCCAGATATAC
GCGTTGACATTGATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACG
TCAATGGGTGGACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCTCTG
GCTAACTAGAGAACCCACTGCTTACTGGCTTATCGAAATGAGACCCAAGCTGGCTAGTTAAGCTAT
CAACAAGTTTGTACAAAAAAGCAGGCTTTAAAGGAACCAATTCAGTCGACTGGATCCGGTACCAC
CATGTTCCTGAACTGCTGCCCAGGTTGCTGTATGGAGCCTGAATTCACCATGGTGAGCAAGGGCGA
GGAGCTGTTCACCGGGGTGGTGCCCATCCTGGTCGAGCTGGACGGCGACGTAAACGGCCACAAGT
TCAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTGGGGCGTGCAGTGC
TTCGCCCGCTACCCCGACCACATGAAGCAGCACGACTTCTTCAAGTCCGCCATGCCCGAAGGCTAC
GTCCAGGAGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGACGGCAAC
ATCCTGGGGCACAAGCTGGAGTACAACGCCATCAGCGACAACGTCTATATCACCGCCGACAAGCA
GAAGAACGGCATCAAGGCCAACTTCAAGATCCGCCACAACATCGAGGACGGCAGCGTGCAGCTCG
CCGACCACTACCAGCAGAACACCCCCATCGGCGACGGCCCCGTGCTGCTGCCCGACAACCACTAC
CTGAGCACCCAGTCCAAGCTGAGCAAAGACCCCAACGAGAAGCGCGATCACATGGTCCTGCTGGA
GTTCGTGACCGCCGCCGGGATCACTCTCGGCATGGACGAGCTGTACAAGGTCGACTATCCGTACGA
CGTACCAGACTACGCATAACCGCGGCCGCACTCGAGATATCTAGACCCAGCTTTCTTGTACAAAGT
GGTTGATCTAGAGGGCCCGCGGTTCGAAGGTAAGCCTATCCCTAACCCTCTCCTCGGTCTCGATTC
TACGCGTACCGGTTAGTAATGAGTTTAAACGGGGGAGGCTAACTGAAACACGGAAGGAGACAATA
CCGGAAGGAACCCGCGCTATGACGGCAATAAAAAGACAGAATAAAACGCACGGGTGTTGGGTCG
TTTGTTCATAAACGCGGGGTTCGGTCCCAGGGCTGGCACTCTGTCGATACCCCACCGAGACCCCAT
TGGGGCCAATACGCCCGCGTTTCTTCCTTTTCCCCACCCCACCCCCCAAGTTCGGGTGAAGGCCCA
GGGCTCGCAGCCAACGTCGGGGCGGCAGGCCCTGCCATAGCAGATCCGATTCGACAGATCACTGA
AATGTGTGGGCGTGGCTTAAGGGTGGGAAAGAATATATAAGGTGGGGGTCTTATGTAGTTTTGTAT
CTGTTTTGCAGCAGCCGCCGCCGCCATGAGCACCAACTCGTTTGATGGAAGCATTGTGAGCTCATA
TTTGACAACGCGCATGCCCCCATGGGCCGGGGTGCGTCAGAATGTGATGGGCTCCAGCATTGATG
GTCGCCCCGTCCTGCCCGCAAACTCTACTACCTTGACCTACGAGACCGTGTCTGGAACGCCGTTGG
AGACTGCAGCCTCCGCCGCCGCTTCAGCCGCTGCAGCCACCGCCCGCGGGATTGTGACTGACTTTG
CTTTCCTGAGCCCGCTTGCAAGCAGTGCAGCTTCCCGTTCATCCGCCCGCGATGACAAGTTGACGG
CTCTTTTGGCACAATTGGATTCTTTGACCCGGGAACTTAATGTCGTTTCTCAGCAGCTGTTGGATCT
GCGCCAGCAGGTTTCTGCCCTGAAGGCTTCCTCCCCTCCCAATGCGGTTTAAAACATAAATAAAAA
ACCAGACTCTGTTTGGATTTGGATCAAGCAAGTGTCTTGCTGTCTTTATTTAGGGGTTTTGCGCGCG
CGGTAGGCCCGGGACCAGCGGTCTCGGTCGTTGAGGGTCCTGTGTATTTTTTCCAGGACGTGGTAA
AGGTGACTCTGGATGTTCAGATACATGGGCATAAGCCCGTCTCTGGGGTGGAGGTAGCACCACTG
CAGAGCTTCATGCTGCGGGGTGGTGTTGTAGATGATCCAGTCGTAGCAGGAGCGCTGGGCGTGGT
GCCTAAAAATGTCTTTCAGTAGCAAGCTGATTGCCAGGGGCAGGCCCTTGGTGTAAGTGTTTACAA
AGCGGTTAAGCTGGGATGGGTGCATACGTGGGGATATGAGATGCATCTTGGACTGTATTTTTAGGT
TGGCTATGTTCCCAGCCATATCCCTCCGGGGATTCATGTTGTGCAGAACCACCAGCACAGTGTATC
CGGTGCACTTGGGAAATTTGTCATGTAGCTTAGAAGGAAATGCGTGGAAGAACTTGGAGACGCCC
TTGTGACCTCCAAGATTTTCCATGCATTCGTCCATAATGATGGCAATGGGCCCACGGGCGGCGGCC
TGGGCGAAGATATTTCTGGGATCACTAACGTCATAGTTGTGTTCCAGGATGAGATCGTCATAGGCC
ATTTTTACAAAGCGCGGGCGGAGGGTGCCAGACTGCGGTATAATGGTTCCATCCGGCCCAGGGGC
GTAGTTACCCTCACAGATTTGCATTTCCCACGCTTTGAGTTCAGATGGGGGGATCATGTCTACCTGC
GGGGCGATGAAGAAAACGGTTTCCGGGGTAGGGGAGATCAGCTGGGAAGAAAGCAGGTTCCTGA
GCAGCTGCGACTTACCGCAGCCGGTGGGCCCGTAAATCACACCTATTACCGGCTGCAACTGGTAGT
TAAGAGAGCTGCAGCTGCCGTCATCCCTGAGCAGGGGGGCCACTTCGTTAAGCATGTCCCTGACTC
GCATGTTTTCCCTGACCAAATCCGCCAGAAGGCGCTCGCCGCCCAGCGATAGCAGTTCTTGCAAGG
AAGCAAAGTTTTTCAACGGTTTGAGACCGTCCGCCGTAGGCATGCTTTTGAGCGTTTGACCAAGCA
GTTCCAGGCGGTCCCACAGCTCGGTCACCTGCTCTACGGCATCTCGATCCAGCATATCTCCTCGTTT
CGCGGGTTGGGGCGGCTTTCGCTGTACGGCAGTAGTCGGTGCTCGTCCAGACGGGCCAGGGTCAT
GTCTTTCCACGGGCGCAGGGTCCTCGTCAGCGTAGTCTGGGTCACGGTGAAGGGGTGCGCTCCGGG
CTGCGCGCTGGCCAGGGTGCGCTTGAGGCTGGTCCTGCTGGTGCTGAAGCGCTGCCGGTCTTCGCC
CTGCGCGTCGGCCAGGTAGCATTTGACCATGGTGTCATAGTCCAGCCCCTCCGCGGCGTGGCCCTT
GGCGCGCAGCTTGCCCTTGGAGGAGGCGCCGCACGAGGGGCAGTGCAGACTTTTGAGGGCGTAGA
GCTTGGGCGCGAGAAATACCGATTCCGGGGAGTAGGCATCCGCGCCGCAGGCCCCGCAGACGGTC
TCGCATTCCACGAGCCAGGTGAGCTCTGGCCGTTCGGGGTCAAAAACCAGGTTTCCCCCATGCTTT
TTGATGCGTTTCTTACCTCTGGTTTCCATGAGCCGGTGTCCACGCTCGGTGACGAAAAGGCTGTCC
GTGTCCCCGTATACAGACTTGAGAGGCCTGTCCTCGAGCGGTGTTCCGCGGTCCTCCTCGTATAGA
AACTCGGACCACTCTGAGACAAAGGCTCGCGTCCAGGCCAGCACGAAGGAGGCTAAGTGGGAGG
GGTAGCGGTCGTTGTCCACTAGGGGGTCCACTCGCTCCAGGGTGTGAAGACACATGTCGCCCTCTT
CGGCATCAAGGAAGGTGATTGGTTTGTAGGTGTAGGCCACGTGACCGGGTGTTCCTGAAGGGGGG
CTATAAAAGGGGGTGGGGGCGCGTTCGTCCTCACTCTCTTCCGCATCGCTGTCTGCGAGGGCCAGC
TGTTGGGGTGAGTACTCCCTCTGAAAAGCGGGCATGACTTCTGCGCTAAGATTGTCAGTTTCCAAA
AACGAGGAGGATTTGATATTCACCTGGCCCGCGGTGATGCCTTTGAGGGTGGCCGCATCCATCTGG
TCAGAAAAGACAATCTTTTTGTTGTCAAGCTTGGTGGCAAACGACCCGTAGAGGGCGTTGGACAG
CAACTTGGCGATGGAGCGCAGGGTTTGGTTTTTGTCGCGATCGGCGCGCTCCTTGGCCGCGATGTT
TAGCTGCACGTATTCGCGCGCAACGCACCGCCATTCGGGAAAGACGGTGGTGCGCTCGTCGGGCA
CCAGGTGCACGCGCCAACCGCGGTTGTGCAGGGTGACAAGGTCAACGCTGGTGGCTACCTCTCCG
CGTAGGCGCTCGTTGGTCCAGCAGAGGCGGCCGCCCTTGCGCGAGCAGAATGGCGGTAGGGGGTC
TAGCTGCGTCTCGTCCGGGGGGTCTGCGTCCACGGTAAAGACCCCGGGCAGCAGGCGCGCGTCGA
AGTAGTCTATCTTGCATCCTTGCAAGTCTAGCGCCTGCTGCCATGCGCGGGCGGCAAGCGCGCGCT
CGTATGGGTTGAGTGGGGGACCCCATGGCATGGGGTGGGTGAGCGCGGAGGCGTACATGCCGCAA
ATGTCGTAAACGTAGAGGGGCTCTCTGAGTATTCCAAGATATGTAGGGTAGCATCTTCCACCGCGG
ATGCTGGCGCGCACGTAATCGTATAGTTCGTGCGAGGGAGCGAGGAGGTCGGGACCGAGGTTGCT
ACGGGCGGGCTGCTCTGCTCGGAAGACTATCTGCCTGAAGATGGCATGTGAGTTGGATGATATGGT
TGGACGCTGGAAGACGTTGAAGCTGGCGTCTGTGAGACCTACCGCGTCACGCACGAAGGAGGCGT
AGGAGTCGCGCAGCTTGTTGACCAGCTCGGCGGTGACCTGCACGTCTAGGGCGCAGTAGTCCAGG
GTTTCCTTGATGATGTCATACTTATCCTGTCCCTTTTTTTTCCACAGCTCGCGGTTGAGGACAAACT
CTTCGCGGTCTTTCCAGTACTCTTGGATCGGAAACCCGTCGGCCTCCGAACGGTAAGAGCCTAGCA
TGTAGAACTGGTTGACGGCCTGGTAGGCGCAGCATCCCTTTTCTACGGGTAGCGCGTATGCCTGCG
CGGCCTTCCGGAGCGAGGTGTGGGTGAGCGCAAAGGTGTCCCTGACCATGACCAGCATGAAGGGC
ACGAGCTGCTTCCCAAAGGCCCCCATCCAAGTATAGGTCTCTACATCGTAGGTGACAAAGAGACG
CTCGGTGCGAGGATGCGAGCCGATCGGGAAGAACTGGATCTCCCGCCACCAATTGGAGGAGTGGC
TATTGATGTGGTGAAAGTAGAAGTCCCTGCGACGGGCCGAACACTCGTGCTGGCTTTTGTAAAAAC
GTGCGCAGTACTGGCAGCGGTGCACGGGCTGTACATCCTGCACGAGGTTGACCTGACGACCGCGC
ACAAGGAAGCAGAGTGGGAATTTGAGCCCCTCGCCTGGCGGGTTTGGCTGGTGGTCTTCTACTTCG
GCTGCTTGTCCTTGACCGTCTGGCTGCTCGAGGGGAGTTACGGTGGATCGGACCACCACGCCGCGC
GAGCCCAAAGTCCAGATGTCCGCGCGCGGCGGTCGGAGCTTGATGACAACATCGCGCAGATGGGA
GCTGTCCATGGTCTGGAGCTCCCGCGGCGTCAGGTCAGGCGGGAGCTCCTGCAGGTTTACCTCGCA
TAGACGGGTCAGGGCGCGGGCTAGATCCAGGTGATACCTAATTTCCAGGGGCTGGTTGGTGGCGG
CGTCGATGGCTTGCAAGAGGCCGCATCCCCGCGGCGCGACTACGGTACCGCGCGGCGGGCGGTGG
GCCGCGGGGGTGTCCTTGGATGATGCATCTAAAAGCGGTGACGCGGGCGAGCCCCCGGAGGTAGG
GGGGGCTCCGGACCCGCCGGGAGAGGGGGCAGGGGCACGTCGGCGCCGCGCGCGGGCAGGAGCT
GGTGCTGCGCGCGTAGGTTGCTGGCGAACGCGACGACGCGGCGGTTGATCTCCTGAATCTGGCGC
CTCTGCGTGAAGACGACGGGCCCGGTGAGCTTGAACCTGAAAGAGAGTTCGACAGAATCAATTTC
GGTGTCGTTGACGGCGGCCTGGCGCAAAATCTCCTGCACGTCTCCTGAGTTGTCTTGATAGGCGAT
CTCGGCCATGAACTGCTCGATCTCTTCCTCCTGGAGATCTCCGCGTCCGGCTCGCTCCACGGTGGC
GGCGAGGTCGTTGGAAATGCGGGCCATGAGCTGCGAGAAGGCGTTGAGGCCTCCCTCGTTCCAGA
CGCGGCTGTAGACCACGCCCCCTTCGGCATCGCGGGCGCGCATGACCACCTGCGCGAGATTGAGC
TCCACGTGCCGGGCGAAGACGGCGTAGTTTCGCAGGCGCTGAAAGAGGTAGTTGAGGGTGGTGGC
GGTGTGTTCTGCCACGAAGAAGTACATAACCCAGCGTCGCAACGTGGATTCGTTGATATCCCCCAA
GGCCTCAAGGCGCTCCATGGCCTCGTAGAAGTCCACGGCGAAGTTGAAAAACTGGGAGTTGCGCG
CCGACACGGTTAACTCCTCCTCCAGAAGACGGATGAGCTCGGCGACAGTGTCGCGCACCTCGCGCT
CAAAGGCTACAGGGGCCTCTTCTTCTTCTTCAATCTCCTCTTCCATAAGGGCCTCCCCTTCTTCTTCT
TCTGGCGGCGGTGGGGGAGGGGGGACACGGCGGCGACGACGGCGCACCGGGAGGCGGTCGACAA
AGCGCTCGATCATCTCCCCGCGGCGACGGCGCATGGTCTCGGTGACGGCGCGGCCGTTCTCGCGGG
GGCGCAGTTGGAAGACGCCGCCCGTCATGTCCCGGTTATGGGTTGGCGGGGGGCTGCCATGCGGC
AGGGATACGGCGCTAACGATGCATCTCAACAATTGTTGTGTAGGTACTCCGCCGCCGAGGGACCT
GAGCGAGTCCGCATCGACCGGATCGGAAAACCTCTCGAGAAAGGCGTCTAACCAGTCACAGTCGC
AAGGTAGGCTGAGCACCGTGGCGGGCGGCAGCGGGCGGCGGTCGGGGTTGTTTCTGGCGGAGGTG
CTGCTGATGATGTAATTAAAGTAGGCGGTCTTGAGACGGCGGATGGTCGACAGAAGCACCATGTC
CTTGGGTCCGGCCTGCTGAATGCGCAGGCGGTCGGCCATGCCCCAGGCTTCGTTTTGACATCGGCG
CAGGTCTTTGTAGTAGTCTTGCATGAGCCTTTCTACCGGCACTTCTTCTTCTCCTTCCTCTTGTCCTG
CATCTCTTGCATCTATCGCTGCGGCGGCGGCGGAGTTTGGCCGTAGGTGGCGCCCTCTTCCTCCCAT
GCGTGTGACCCCGAAGCCCCTCATCGGCTGAAGCAGGGCTAGGTCGGCGACAACGCGCTCGGCTA
ATATGGCCTGCTGCACCTGCGTGAGGGTAGACTGGAAGTCATCCATGTCCACAAAGCGGTGGTAT
GCGCCCGTGTTGATGGTGTAAGTGCAGTTGGCCATAACGGACCAGTTAACGGTCTGGTGACCCGGC
TGCGAGAGCTCGGTGTACCTGAGACGCGAGTAAGCCCTCGAGTCAAATACGTAGTCGTTGCAAGT
CCGCACCAGGTACTGGTATCCCACCAAAAAGTGCGGCGGCGGCTGGCGGTAGAGGGGCCAGCGTA
GGGTGGCCGGGGCTCCGGGGGCGAGATCTTCCAACATAAGGCGATGATATCCGTAGATGTACCTG
GACATCCAGGTGATGCCGGCGGCGGTGGTGGAGGCGCGCGGAAAGTCGCGGACGCGGTTCCAGAT
GTTGCGCAGCGGCAAAAAGTGCTCCATGGTCGGGACGCTCTGGCCGGTCAGGCGCGCGCAATCGT
TGACGCTCTAGACCGTGCAAAAGGAGAGCCTGTAAGCGGGCACTCTTCCGTGGTCTGGTGGATAA
ATTCGCAAGGGTATCATGGCGGACGACCGGGGTTCGAGCCCCGTATCCGGCCGTCCGCCGTGATCC
ATGCGGTTACCGCCCGCGTGTCGAACCCAGGTGTGCGACGTCAGACAACGGGGGAGTGCTCCTTTT
GGCTTCCTTCCAGGCGCGGCGGCTGCTGCGCTAGCTTTTTTGGCCACTGGCCGCGCGCAGCGTAAG
CGGTTAGGCTGGAAAGCGAAAGCATTAAGTGGCTCGCTCCCTGTAGCCGGAGGGTTATTTTCCAAG
GGTTGAGTCGCGGGACCCCCGGTTCGAGTCTCGGACCGGCCGGACTGCGGCGAACGGGGGTTTGC
CTCCCCGTCATGCAAGACCCCGCTTGCAAATTCCTCCGGAAACAGGGACGAGCCCCTTTTTTGCTT
TTCCCAGATGCATCCGGTGCTGCGGCAGATGCGCCCCCCTCCTCAGCAGCGGCAAGAGCAAGAGC
AGCGGCAGACATGCAGGGCACCCTCCCCTCCTCCTACCGCGTCAGGAGGGGCGACATCCGCGGTT
GACGCGGCAGCAGATGGTGATTACGAACCCCCGCGGCGCCGGGCCCGGCACTACCTGGACTTGGA
GGAGGGCGAGGGCCTGGCGCGGCTAGGAGCGCCCTCTCCTGAGCGGCACCCAAGGGTGCAGCTGA
AGCGTGATACGCGTGAGGCGTACGTGCCGCGGCAGAACCTGTTTCGCGACCGCGAGGGAGAGGAG
CCCGAGGAGATGCGGGATCGAAAGTTCCACGCAGGGCGCGAGCTGCGGCATGGCCTGAATCGCGA
GCGGTTGCTGCGCGAGGAGGACTTTGAGCCCGACGCGCGAACCGGGATTAGTCCCGCGCGCGCAC
ACGTGGCGGCCGCCGACCTGGTAACCGCATACGAGCAGACGGTGAACCAGGAGATTAACTTTCAA
AAAAGCTTTAACAACCACGTGCGTACGCTTGTGGCGCGCGAGGAGGTGGCTATAGGACTGATGCA
TCTGTGGGACTTTGTAAGCGCGCTGGAGCAAAACCCAAATAGCAAGCCGCTCATGGCGCAGCTGT
TCCTTATAGTGCAGCACAGCAGGGACAACGAGGCATTCAGGGATGCGCTGCTAAACATAGTAGAG
CCCGAGGGCCGCTGGCTGCTCGATTTGATAAACATCCTGCAGAGCATAGTGGTGCAGGAGCGCAG
CTTGAGCCTGGCTGACAAGGTGGCCGCCATCAACTATTCCATGCTTAGCCTGGGCAAGTTTTACGC
CCGCAAGATATACCATACCCCTTACGTTCCCATAGACAAGGAGGTAAAGATCGAGGGGTTCTACA
TGCGCATGGCGCTGAAGGTGCTTACCTTGAGCGACGACCTGGGCGTTTATCGCAACGAGCGCATCC
ACAAGGCCGTGAGCGTGAGCCGGCGGCGCGAGCTCAGCGACCGCGAGCTGATGCACAGCCTGCAA
AGGGCCCTGGCTGGCACGGGCAGCGGCGATAGAGAGGCCGAGTCCTACTTTGACGCGGGCGCTGA
CCTGCGCTGGGCCCCAAGCCGACGCGCCCTGGAGGCAGCTGGGGCCGGACCTGGGCTGGCGGTGG
CACCCGCGCGCGCTGGCAACGTCGGCGGCGTGGAGGAATATGACGAGGACGATGAGTACGAGCC
AGAGGACGGCGAGTACTAAGCGGTGATGTTTCTGATCAGATGATGCAAGACGCAACGGACCCGGC
GGTGCGGGCGGCGCTGCAGAGCCAGCCGTCCGGCCTTAACTCCACGGACGACTGGCGCCAGGTCA
TGGACCGCATCATGTCGCTGACTGCGCGCAATCCTGACGCGTTCCGGCAGCAGCCGCAGGCCAAC
CGGCTCTCCGCAATTCTGGAAGCGGTGGTCCCGGCGCGCGCAAACCCCACGCACGAGAAGGTGCT
GGCGATCGTAAACGCGCTGGCCGAAAACAGGGCCATCCGGCCCGACGAGGCCGGCCTGGTCTACG
ACGCGCTGCTTCAGCGCGTGGCTCGTTACAACAGCGGCAACGTGCAGACCAACCTGGACCGGCTG
GTGGGGGATGTGCGCGAGGCCGTGGCGCAGCGTGAGCGCGCGCAGCAGCAGGGCAACCTGGGCT
CCATGGTTGCACTAAACGCCTTCCTGAGTACACAGCCCGCCAACGTGCCGCGGGGACAGGAGGAC
TACACCAACTTTGTGAGCGCACTGCGGCTAATGGTGACTGAGACACCGCAAAGTGAGGTGTACCA
GTCTGGGCCAGACTATTTTTTCCAGACCAGTAGACAAGGCCTGCAGACCGTAAACCTGAGCCAGG
CTTTCAAAAACTTGCAGGGGCTGTGGGGGGTGCGGGCTCCCACAGGCGACCGCGCGACCGTGTCT
AGCTTGCTGACGCCCAACTCGCGCCTGTTGCTGCTGCTAATAGCGCCCTTCACGGACAGTGGCAGC
GTGTCCCGGGACACATACCTAGGTCACTTGCTGACACTGTACCGCGAGGCCATAGGTCAGGCGCAT
GTGGACGAGCATACTTTCCAGGAGATTACAAGTGTCAGCCGCGCGCTGGGGCAGGAGGACACGGG
CAGCCTGGAGGCAACCCTAAACTACCTGCTGACCAACCGGCGGCAGAAGATCCCCTCGTTGCACA
GTTTAAACAGCGAGGAGGAGCGCATTTTGCGCTACGTGCAGCAGAGCGTGAGCCTTAACCTGATG
CGCGACGGGGTAACGCCCAGCGTGGCGCTGGACATGACCGCGCGCAACATGGAACCGGGCATGTA
TGCCTCAAACCGGCCGTTTATCAACCGCCTAATGGACTACTTGCATCGCGCGGCCGCCGTGAACCC
CGAGTATTTCACCAATGCCATCTTGAACCCGCACTGGCTACCGCCCCCTGGTTTCTACACCGGGGG
ATTCGAGGTGCCCGAGGGTAACGATGGATTCCTCTGGGACGACATAGACGACAGCGTGTTTTCCCC
GCAACCGCAGACCCTGCTAGAGTTGCAACAGCGCGAGCAGGCAGAGGCGGCGCTGCGAAAGGAA
AGCTTCCGCAGGCCAAGCAGCTTGTCCGATCTAGGCGCTGCGGCCCCGCGGTCAGATGCTAGTAGC
CCATTTCCAAGCTTGATAGGGTCTCTTACCAGCACTCGCACCACCCGCCCGCGCCTGCTGGGCGAG
GAGGAGTACCTAAACAACTCGCTGCTGCAGCCGCAGCGCGAAAAAAACCTGCCTCCGGCATTTCC
CAACAACGGGATAGAGAGCCTAGTGGACAAGATGAGTAGATGGAAGACGTACGCGCAGGAGCAC
AGGGACGTGCCAGGCCCGCGCCCGCCCACCCGTCGTCAAAGGCACGACCGTCAGCGGGGTCTGGT
GTGGGAGGACGATGACTCGGCAGACGACAGCAGCGTCCTGGATTTGGGAGGGAGTGGCAACCCGT
TTGCGCACCTTCGCCCCAGGCTGGGGAGAATGTTTTAAAAAAAAAAAAAGCATGATGCAAAATAA
AAAACTCACCAAGGCCATGGCACCGAGCGTTGGTTTTCTTGTATTCCCCTTAGTATGCGGCGCGCG
GCGATGTATGAGGAAGGTCCTCCTCCCTCCTACGAGAGTGTGGTGAGCGCGGCGCCAGTGGCGGC
GGCGCTGGGTTCTCCCTTCGATGCTCCCCTGGACCCGCCGTTTGTGCCTCCGCGGTACCTGCGGCCT
ACCGGGGGGAGAAACAGCATCCGTTACTCTGAGTTGGCACCCCTATTCGACACCACCCGTGTGTAC
CTGGTGGACAACAAGTCAACGGATGTGGCATCCCTGAACTACCAGAACGACCACAGCAACTTTCT
GACCACGGTCATTCAAAACAATGACTACAGCCCGGGGGAGGCAAGCACACAGACCATCAATCTTG
ACGACCGGTCGCACTGGGGCGGCGACCTGAAAACCATCCTGCATACCAACATGCCAAATGTGAAC
GAGTTCATGTTTACCAATAAGTTTAAGGCGCGGGTGATGGTGTCGCGCTTGCCTACTAAGGACAAT
CAGGTGGAGCTGAAATACGAGTGGGTGGAGTTCACGCTGCCCGAGGGCAACTACTCCGAGACCAT
GACCATAGACCTTATGAACAACGCGATCGTGGAGCACTACTTGAAAGTGGGCAGACAGAACGGGG
TTCTGGAAAGCGACATCGGGGTAAAGTTTGACACCCGCAACTTCAGACTGGGGTTTGACCCCGTCA
CTGGTCTTGTCATGCCTGGGGTATATACAAACGAAGCCTTCCATCCAGACATCATTTTGCTGCCAG
GATGCGGGGTGGACTTCACCCACAGCCGCCTGAGCAACTTGTTGGGCATCCGCAAGCGGCAACCC
TTCCAGGAGGGCTTTAGGATCACCTACGATGATCTGGAGGGTGGTAACATTCCCGCACTGTTGGAT
GTGGACGCCTACCAGGCGAGCTTGAAAGATGACACCGAACAGGGCGGGGGTGGCGCAGGCGGCA
GCAACAGCAGTGGCAGCGGCGCGGAAGAGAACTCCAACGCGGCAGCCGCGGCAATGCAGCCGGT
GGAGGACATGAACGATCATGCCATTCGCGGCGACACCTTTGCCACACGGGCTGAGGAGAAGCGCG
CTGAGGCCGAAGCAGCGGCCGAAGCTGCCGCCCCCGCTGCGCAACCCGAGGTCGAGAAGCCTCAG
AAGAAACCGGTGATCAAACCCCTGACAGAGGACAGCAAGAAACGCAGTTACAACCTAATAAGCA
ATGACAGCACCTTCACCCAGTACCGCAGCTGGTACCTTGCATACAACTACGGCGACCCTCAGACCG
GAATCCGCTCATGGACCCTGCTTTGCACTCCTGACGTAACCTGCGGCTCGGAGCAGGTCTACTGGT
CGTTGCCAGACATGATGCAAGACCCCGTGACCTTCCGCTCCACGCGCCAGATCAGCAACTTTCCGG
TGGTGGGCGCCGAGCTGTTGCCCGTGCACTCCAAGAGCTTCTACAACGACCAGGCCGTCTACTCCC
AACTCATCCGCCAGTTTACCTCTCTGACCCACGTGTTCAATCGCTTTCCCGAGAACCAGATTTTGGC
GCGCCCGCCAGCCCCCACCATCACCACCGTCAGTGAAAACGTTCCTGCTCTCACAGATCACGGGAC
GCTACCGCTGCGCAACAGCATCGGAGGAGTCCAGCGAGTGACCATTACTGACGCCAGACGCCGCA
CCTGCCCCTACGTTTACAAGGCCCTGGGCATAGTCTCGCCGCGCGTCCTATCGAGCCGCACTTTTTG
AGCAAGCATGTCCATCCTTATATCGCCCAGCAATAACACAGGCTGGGGCCTGCGCTTCCCAAGCAA
GATGTTTGGCGGGGCCAAGAAGCGCTCCGACCAACACCCAGTGCGCGTGCGCGGGCACTACCGCG
CGCCCTGGGGCGCGCACAAACGCGGCCGCACTGGGCGCACCACCGTCGATGACGCCATCGACGCG
GTGGTGGAGGAGGCGCGCAACTACACGCCCACGCCGCCACCAGTGTCCACAGTGGACGCGGCCAT
TCAGACCGTGGTGCGCGGAGCCCGGCGCTATGCTAAAATGAAGAGACGGCGGAGGCGCGTAGCAC
GTCGCCACCGCCGCCGACCCGGCACTGCCGCCCAACGCGCGGCGGCGGCCCTGCTTAACCGCGCA
CGTCGCACCGGCCGACGGGCGGCCATGCGGGCCGCTCGAAGGCTGGCCGCGGGTATTGTCACTGT
GCCCCCCAGGTCCAGGCGACGAGCGGCCGCCGCAGCAGCCGCGGCCATTAGTGCTATGACTCAGG
GTCGCAGGGGCAACGTGTATTGGGTGCGCGACTCGGTTAGCGGCCTGCGCGTGCCCGTGCGCACC
CGCCCCCCGCGCAACTAGATTGCAAGAAAAAACTACTTAGACTCGTACTGTTGTATGTATCCAGCG
GCGGCGGCGCGCAACGAAGCTATGTCCAAGCGCAAAATCAAAGAAGAGATGCTCCAGGTCATCGC
GCCGGAGATCTATGGCCCCCCGAAGAAGGAAGAGCAGGATTACAAGCCCCGAAAGCTAAAGCGG
GTCAAAAAGAAAAAGAAAGATGATGATGATGAACTTGACGACGAGGTGGAACTGCTGCACGCTA
CCGCGCCCAGGCGACGGGTACAGTGGAAAGGTCGACGCGTAAAACGTGTTTTGCGACCCGGCACC
ACCGTAGTCTTTACGCCCGGTGAGCGCTCCACCCGCACCTACAAGCGCGTGTATGATGAGGTGTAC
GGCGACGAGGACCTGCTTGAGCAGGCCAACGAGCGCCTCGGGGAGTTTGCCTACGGAAAGCGGCA
TAAGGACATGCTGGCGTTGCCGCTGGACGAGGGCAACCCAACACCTAGCCTAAAGCCCGTAACAC
TGCAGCAGGTGCTGCCCGCGCTTGCACCGTCCGAAGAAAAGCGCGGCCTAAAGCGCGAGTCTGGT
GACTTGGCACCCACCGTGCAGCTGATGGTACCCAAGCGCCAGCGACTGGAAGATGTCTTGGAAAA
AATGACCGTGGAACCTGGGCTGGAGCCCGAGGTCCGCGTGCGGCCAATCAAGCAGGTGGCGCCGG
GACTGGGCGTGCAGACCGTGGACGTTCAGATACCCACTACCAGTAGCACCAGTATTGCCACCGCC
ACAGAGGGCATGGAGACACAAACGTCCCCGGTTGCCTCAGCGGTGGCGGATGCCGCGGTGCAGGC
GGTCGCTGCGGCCGCGTCCAAGACCTCTACGGAGGTGCAAACGGACCCGTGGATGTTTCGCGTTTC
AGCCCCCCGGCGCCCGCGCCGTTCGAGGAAGTACGGCGCCGCCAGCGCGCTACTGCCCGAATATG
CCCTACATCCTTCCATTGCGCCTACCCCCGGCTATCGTGGCTACACCTACCGCCCCAGAAGACGAG
CAACTACCCGACGCCGAACCACCACTGGAACCCGCCGCCGCCGTCGCCGTCGCCAGCCCGTGCTG
GCCCCGATTTCCGTGCGCAGGGTGGCTCGCGAAGGAGGCAGGACCCTGGTGCTGCCAACAGCGCG
CTACCACCCCAGCATCGTTTAAAAGCCGGTCTTTGTGGTTCTTGCAGATATGGCCCTCACCTGCCGC
CTCCGTTTCCCGGTGCCGGGATTCCGAGGAAGAATGCACCGTAGGAGGGGCATGGCCGGCCACGG
CCTGACGGGCGGCATGCGTCGTGCGCACCACCGGCGGCGGCGCGCGTCGCACCGTCGCATGCGCG
GCGGTATCCTGCCCCTCCTTATTCCACTGATCGCCGCGGCGATTGGCGCCGTGCCCGGAATTGCAT
CCGTGGCCTTGCAGGCGCAGAGACACTGATTAAAAACAAGTTGCATGTGGAAAAATCAAAATAAA
AAGTCTGGACTCTCACGCTCGCTTGGTCCTGTAACTATTTTGTAGAATGGAAGACATCAACTTTGC
GTCTCTGGCCCCGCGACACGGCTCGCGCCCGTTCATGGGAAACTGGCAAGATATCGGCACCAGCA
ATATGAGCGGTGGCGCCTTCAGCTGGGGCTCGCTGTGGAGCGGCATTAAAAATTTCGGTTCCACCG
TTAAGAACTATGGCAGCAAGGCCTGGAACAGCAGCACAGGCCAGATGCTGAGGGATAAGTTGAA
AGAGCAAAATTTCCAACAAAAGGTGGTAGATGGCCTGGCCTCTGGCATTAGCGGGGTGGTGGACC
TGGCCAACCAGGCAGTGCAAAATAAGATTAACAGTAAGCTTGATCCCCGCCCTCCCGTAGAGGAG
CCTCCACCGGCCGTGGAGACAGTGTCTCCAGAGGGGCGTGGCGAAAAGCGTCCGCGCCCCGACAG
GGAAGAAACTCTGGTGACGCAAATAGACGAGCCTCCCTCGTACGAGGAGGCACTAAAGCAAGGCC
TGCCCACCACCCGTCCCATCGCGCCCATGGCTACCGGAGTGCTGGGCCAGCACACACCCGTAACGC
TGGACCTGCCTCCCCCCGCCGACACCCAGCAGAAACCTGTGCTGCCAGGCCCGACCGCCGTTGTTG
TAACCCGTCCTAGCCGCGCGTCCCTGCGCCGCGCCGCCAGCGGTCCGCGATCGTTGCGGCCCGTAG
CCAGTGGCAACTGGCAAAGCACACTGAACAGCATCGTGGGTCTGGGGGTGCAATCCCTGAAGCGC
CGACGATGCTTCTGATAGCTAACGTGTCGTATGTGTGTCATGTATGCGTCCATGTCGCCGCCAGAG
GAGCTGCTGAGCCGCCGCGCGCCCGCTTTCCAAGATGGCTACCCCTTCGATGATGCCGCAGTGGTC
TTACATGCACATCTCGGGCCAGGACGCCTCGGAGTACCTGAGCCCCGGGCTGGTGCAGTTTGCCCG
CGCCACCGAGACGTACTTCAGCCTGAATAACAAGTTTAGAAACCCCACGGTGGCGCCTACGCACG
ACGTGACCACAGACCGGTCCCAGCGTTTGACGCTGCGGTTCATCCCTGTGGACCGTGAGGATACTG
CGTACTCGTACAAGGCGCGGTTCACCCTAGCTGTGGGTGATAACCGTGTGCTGGACATGGCTTCCA
CGTACTTTGACATCCGCGGCGTGCTGGACAGGGGCCCTACTTTTAAGCCCTACTCTGGCACTGCCT
ACAACGCCCTGGCTCCCAAGGGTGCCCCAAATCCTTGCGAATGGGATGAAGCTGCTACTGCTCTTG
AAATAAACCTAGAAGAAGAGGACGATGACAACGAAGACGAAGTAGACGAGCAAGCTGAGCAGCA
AAAAACTCACGTATTTGGGCAGGCGCCTTATTCTGGTATAAATATTACAAAGGAGGGTATTCAAAT
AGGTGTCGAAGGTCAAACACCTAAATATGCCGATAAAACATTTCAACCTGAACCTCAAATAGGAG
AATCTCAGTGGTACGAAACAGAAATTAATCATGCAGCTGGGAGAGTCCTAAAAAAGACTACCCCA
ATGAAACCATGTTACGGTTCATATGCAAAACCCACAAATGAAAATGGAGGGCAAGGCATTCTTGT
AAAGCAACAAAATGGAAAGCTAGAAAGTCAAGTGGAAATGCAATTTTTCTCAACTACTGAGGCAG
CCGCAGGCAATGGTGATAACTTGACTCCTAAAGTGGTATTGTACAGTGAAGATGTAGATATAGAA
ACCCCAGACACTCATATTTCTTACATGCCCACTATTAAGGAAGGTAACTCACGAGAACTAATGGGC
CAACAATCTATGCCCAACAGGCCTAATTACATTGCTTTTAGGGACAATTTTATTGGTCTAATGTATT
ACAACAGCACGGGTAATATGGGTGTTCTGGCGGGCCAAGCATCGCAGTTGAATGCTGTTGTAGATT
TGCAAGACAGAAACACAGAGCTTTCATACCAGCTTTTGCTTGATTCCATTGGTGATAGAACCAGGT
ACTTTTCTATGTGGAATCAGGCTGTTGACAGCTATGATCCAGATGTTAGAATTATTGAAAATCATG
GAACTGAAGATGAACTTCCAAATTACTGCTTTCCACTGGGAGGTGTGATTAATACAGAGACTCTTA
CCAAGGTAAAACCTAAAACAGGTCAGGAAAATGGATGGGAAAAAGATGCTACAGAATTTTCAGAT
AAAAATGAAATAAGAGTTGGAAATAATTTTGCCATGGAAATCAATCTAAATGCCAACCTGTGGAG
AAATTTCCTGTACTCCAACATAGCGCTGTATTTGCCCGACAAGCTAAAGTACAGTCCTTCCAACGT
AAAAATTTCTGATAACCCAAACACCTACGACTACATGAACAAGCGAGTGGTGGCTCCCGGGCTAG
TGGACTGCTACATTAACCTTGGAGCACGCTGGTCCCTTGACTATATGGACAACGTCAACCCATTTA
ACCACCACCGCAATGCTGGCCTGCGCTACCGCTCAATGTTGCTGGGCAATGGTCGCTATGTGCCCT
TCCACATCCAGGTGCCTCAGAAGTTCTTTGCCATTAAAAACCTCCTTCTCCTGCCGGGCTCATACAC
CTACGAGTGGAACTTCAGGAAGGATGTTAACATGGTTCTGCAGAGCTCCCTAGGAAATGACCTAA
GGGTTGACGGAGCCAGCATTAAGTTTGATAGCATTTGCCTTTACGCCACCTTCTTCCCCATGGCCC
ACAACACCGCCTCCACGCTTGAGGCCATGCTTAGAAACGACACCAACGACCAGTCCTTTAACGACT
ATCTCTCCGCCGCCAACATGCTCTACCCTATACCCGCCAACGCTACCAACGTGCCCATATCCATCC
CCTCCCGCAACTGGGCGGCTTTCCGCGGCTGGGCCTTCACGCGCCTTAAGACTAAGGAAACCCCAT
CACTGGGCTCGGGCTACGACCCTTATTACACCTACTCTGGCTCTATACCCTACCTAGATGGAACCTT
TTACCTCAACCACACCTTTAAGAAGGTGGCCATTACCTTTGACTCTTCTGTCAGCTGGCCTGGCAAT
GACCGCCTGCTTACCCCCAACGAGTTTGAAATTAAGCGCTCAGTTGACGGGGAGGGTTACAACGTT
GCCCAGTGTAACATGACCAAAGACTGGTTCCTGGTACAAATGCTAGCTAACTATAACATTGGCTAC
CAGGGCTTCTATATCCCAGAGAGCTACAAGGACCGCATGTACTCCTTCTTTAGAAACTTCCAGCCC
ATGAGCCGTCAGGTGGTGGATGATACTAAATACAAGGACTACCAACAGGTGGGCATCCTACACCA
ACACAACAACTCTGGATTTGTTGGCTACCTTGCCCCCACCATGCGCGAAGGACAGGCCTACCCTGC
TAACTTCCCCTATCCGCTTATAGGCAAGACCGCAGTTGACAGCATTACCCAGAAAAAGTTTCTTTG
CGATCGCACCCTTTGGCGCATCCCATTCTCCAGTAACTTTATGTCCATGGGCGCACTCACAGACCT
GGGCCAAAACCTTCTCTACGCCAACTCCGCCCACGCGCTAGACATGACTTTTGAGGTGGATCCCAT
GGACGAGCCCACCCTTCTTTATGTTTTGTTTGAAGTCTTTGACGTGGTCCGTGTGCACCAGCCGCAC
CGCGGCGTCATCGAAACCGTGTACCTGCGCACGCCCTTCTCGGCCGGCAACGCCACAACATAAAG
AAGCAAGCAACATCAACAACAGCTGCCGCCATGGGCTCCAGTGAGCAGGAACTGAAAGCCATTGT
CAAAGATCTTGGTTGTGGGCCATATTTTTTGGGCACCTATGACAAGCGCTTTCCAGGCTTTGTTTCT
CCACACAAGCTCGCCTGCGCCATAGTCAATACGGCCGGTCGCGAGACTGGGGGCGTACACTGGAT
GGCCTTTGCCTGGAACCCGCACTCAAAAACATGCTACCTCTTTGAGCCCTTTGGCTTTTCTGACCAG
CGACTCAAGCAGGTTTACCAGTTTGAGTACGAGTCACTCCTGCGCCGTAGCGCCATTGCTTCTTCC
CCCGACCGCTGTATAACGCTGGAAAAGTCCACCCAAAGCGTACAGGGGCCCAACTCGGCCGCCTG
TGGACTATTCTGCTGCATGTTTCTCCACGCCTTTGCCAACTGGCCCCAAACTCCCATGGATCACAAC
CCCACCATGAACCTTATTACCGGGGTACCCAACTCCATGCTCAACAGTCCCCAGGTACAGCCCACC
CTGCGTCGCAACCAGGAACAGCTCTACAGCTTCCTGGAGCGCCACTCGCCCTACTTCCGCAGCCAC
AGTGCGCAGATTAGGAGCGCCACTTCTTTTTGTCACTTGAAAAACATGTAAAAATAATGTACTAGA
GACACTTTCAATAAAGGCAAATGCTTTTATTTGTACACTCTCGGGTGATTATTTACCCCCACCCTTG
CCGTCTGCGCCGTTTAAAAATCAAAGGGGTTCTGCCGCGCATCGCTATGCGCCACTGGCAGGGACA
CGTTGCGATACTGGTGTTTAGTGCTCCACTTAAACTCAGGCACAACCATCCGCGGCAGCTCGGTGA
AGTTTTCACTCCACAGGCTGCGCACCATCACCAACGCGTTTAGCAGGTCGGGCGCCGATATCTTGA
AGTCGCAGTTGGGGCCTCCGCCCTGCGCGCGCGAGTTGCGATACACAGGGTTGCAGCACTGGAAC
ACTATCAGCGCCGGGTGGTGCACGCTGGCCAGCACGCTCTTGTCGGAGATCAGATCCGCGTCCAG
GTCCTCCGCGTTGCTCAGGGCGAACGGAGTCAACTTTGGTAGCTGCCTTCCCAAAAAGGGCGCGTG
CCCAGGCTTTGAGTTGCACTCGCACCGTAGTGGCATCAAAAGGTGACCGTGCCCGGTCTGGGCGTT
AGGATACAGCGCCTGCATAAAAGCCTTGATCTGCTTAAAAGCCACCTGAGCCTTTGCGCCTTCAGA
GAAGAACATGCCGCAAGACTTGCCGGAAAACTGATTGGCCGGACAGGCCGCGTCGTGCACGCAGC
ACCTTGCGTCGGTGTTGGAGATCTGCACCACATTTCGGCCCCACCGGTTCTTCACGATCTTGGCCTT
GCTAGACTGCTCCTTCAGCGCGCGCTGCCCGTTTTCGCTCGTCACATCCATTTCAATCACGTGCTCC
TTATTTATCATAATGCTTCCGTGTAGACACTTAAGCTCGCCTTCGATCTCAGCGCAGCGGTGCAGCC
ACAACGCGCAGCCCGTGGGCTCGTGATGCTTGTAGGTCACCTCTGCAAACGACTGCAGGTACGCCT
GCAGGAATCGCCCCATCATCGTCACAAAGGTCTTGTTGCTGGTGAAGGTCAGCTGCAACCCGCGGT
GCTCCTCGTTCAGCCAGGTCTTGCATACGGCCGCCAGAGCTTCCACTTGGTCAGGCAGTAGTTTGA
AGTTCGCCTTTAGATCGTTATCCACGTGGTACTTGTCCATCAGCGCGCGCGCAGCCTCCATGCCCTT
CTCCCACGCAGACACGATCGGCACACTCAGCGGGTTCATCACCGTAATTTCACTTTCCGCTTCGCT
GGGCTCTTCCTCTTCCTCTTGCGTCCGCATACCACGCGCCACTGGGTCGTCTTCATTCAGCCGCCGC
ACTGTGCGCTTACCTCCTTTGCCATGCTTGATTAGCACCGGTGGGTTGCTGAAACCCACCATTTGTA
GCGCCACATCTTCTCTTTCTTCCTCGCTGTCCACGATTACTTTCACAGGAGGTACAGCTATGACCAT
GATTACGGATTCACTGGCCGTCGTTTTACAACGTCGTGACTGGGAAAACCCTGGCGTTACCCAACT
TAATCGCCTTGCAGCACATCCCCCTTTCGCCAGCTGGCGTAATAGCGAAGAGGCCCGCACCGATCG
CCCTTCCCAACAGTTGCGCAGCCTGAATGGCGAATAGGTCGCGCCGCACCGCGTCCGCGCTCGGG
GGTGGTTTCGCGCTGCTCCTCTTCCCGACTGGCCATTTCCTTCTCCTATAGGCAGAAAAAGATCATG
GAGTCAGTCGAGAAGAAGGACAGCCTAACCGCCCCCTCTGAGTTCGCCACCACCGCCTCCACCGA
TGCCGCCAACGCGCCTACCACCTTCCCCGTCGAGGCACCCCCGCTTGAGGAGGAGGAAGTGATTAT
CGAGCAGGACCCAGGTTTTGTAAGCGAAGACGACGAGGACCGCTCAGTACCAACAGAGGATAAA
AAGCAAGACCAGGACAACGCAGAGGCAAACGAGGAACAAGTCGGGCGGGGGGACGAAAGGCAT
GGCGACTACCTAGATGTGGGAGACGACGTGCTGTTGAAGCATCTGCAGCGCCAGTGCGCCATTAT
CTGCGACGCGTTGCAAGAGCGCAGCGATGTGCCCCTCGCCATAGCGGATGTCAGCCTTGCCTACGA
ACGCCACCTATTCTCACCGCGCGTACCCCCCAAACGCCAAGAAAACGGCACATGCGAGCCCAACC
CGCGCCTCAACTTCTACCCCGTATTTGCCGTGCCAGAGGTGCTTGCCACCTATCACATCTTTTTCCA
AAACTGCAAGATACCCCTATCCTGCCGTGCCAACCGCAGCCGAGCGGACAAGCAGCTGGCCTTGC
GGCAGGGCGCTGTCATACCTGATATCGCCTCGCTCAACGAAGTGCCAAAAATCTTTGAGGGTCTTG
GACGCGACGAGAAGCGCGCGGCAAACGCTCTGCAACAGGAAAACAGCGAAAATGAAAGTCACTC
TGGAGTGTTGGTGGAACTCGAGGGTGACAACGCGCGCCTAGCCGTACTAAAACGCAGCATCGAGG
TCACCCACTTTGCCTACCCGGCACTTAACCTACCCCCCAAGGTCATGAGCACAGTCATGAGTGAGC
TGATCGTGCGCCGTGCGCAGCCCCTGGAGAGGGATGCAAATTTGCAAGAACAAACAGAGGAGGGC
CTACCCGCAGTTGGCGACGAGCAGCTAGCGCGCTGGCTTCAAACGCGCGAGCCTGCCGACTTGGA
GGAGCGACGCAAACTAATGATGGCCGCAGTGCTCGTTACCGTGGAGCTTGAGTGCATGCAGCGGT
TCTTTGCTGACCCGGAGATGCAGCGCAAGCTAGAGGAAACATTGCACTACACCTTTCGACAGGGCT
ACGTACGCCAGGCCTGCAAGATCTCCAACGTGGAGCTCTGCAACCTGGTCTCCTACCTTGGAATTT
TGCACGAAAACCGCCTTGGGCAAAACGTGCTTCATTCCACGCTCAAGGGCGAGGCGCGCCGCGAC
TACGTCCGCGACTGCGTTTACTTATTTCTATGCTACACCTGGCAGACGGCCATGGGCGTTTGGCAG
CAGTGCTTGGAGGAGTGCAACCTCAAGGAGCTGCAGAAACTGCTAAAGCAAAACTTGAAGGACCT
ATGGACGGCCTTCAACGAGCGCTCCGTGGCCGCGCACCTGGCGGACATCATTTTCCCCGAACGCCT
GCTTAAAACCCTGCAACAGGGTCTGCCAGACTTCACCAGTCAAAGCATGTTGCAGAACTTTAGGA
ACTTTATCCTAGAGCGCTCAGGAATCTTGCCCGCCACCTGCTGTGCACTTCCTAGCGACTTTGTGCC
CATTAAGTACCGCGAATGCCCTCCGCCGCTTTGGGGCCACTGCTACCTTCTGCAGCTAGCCAACTA
CCTTGCCTACCACTCTGACATAATGGAAGACGTGAGCGGTGACGGTCTACTGGAGTGTCACTGTCG
CTGCAACCTATGCACCCCGCACCGCTCCCTGGTTTGCAATTCGCAGCTGCTTAACGAAAGTCAAAT
TATCGGTACCTTTGAGCTGCAGGGTCCCTCGCCTGACGAAAAGTCCGCGGCTCCGGGGTTGAAACT
CACTCCGGGGCTGTGGACGTCGGCTTACCTTCGCAAATTTGTACCTGAGGACTACCACGCCCACGA
GATTAGGTTCTACGAAGACCAATCCCGCCCGCCTAATGCGGAGCTTACCGCCTGCGTCATTACCCA
GGGCCACATTCTTGGCCAATTGCAAGCCATCAACAAAGCCCGCCAAGAGTTTCTGCTACGAAAGG
GACGGGGGGTTTACTTGGACCCCCAGTCCGGCGAGGAGCTCAACCCAATCCCCCCGCCGCCGCAG
CCCTATCAGCAGCAGCCGCGGGCCCTTGCTTCCCAGGATGGCACCCAAAAAGAAGCTGCAGCTGC
CGCCGCCACCCACGGACGAGGAGGAATACTGGGACAGTCAGGCAGAGGAGGTTTTGGACGAGGA
GGAGGAGGACATGATGGAAGACTGGGAGAGCCTAGACGAGGAAGCTTCCGAGGTCGAAGAGGTG
TCAGACGAAACACCGTCACCCTCGGTCGCATTCCCCTCGCCGGCGCCCCAGAAATCGGCAACCGGT
TCCAGCATGGCTACAACCTCCGCTCCTCAGGCGCCGCCGGCACTGCCCGTTCGCCGACCCAACCGT
AGATGGGACACCACTGGAACCAGGGCCGGTAAGTCCAAGCAGCCGCCGCCGTTAGCCCAAGAGCA
ACAACAGCGCCAAGGCTACCGCTCATGGCGCGGGCACAAGAACGCCATAGTTGCTTGCTTGCAAG
ACTGTGGGGGCAACATCTCCTTCGCCCGCCGCTTTCTTCTCTACCATCACGGCGTGGCCTTCCCCCG
TAACATCCTGCATTACTACCGTCATCTCTACAGCCCATACTGCACCGGCGGCAGCGGCAGCAACAG
CAGCGGCCACACAGAAGCAAAGGCGACCGGATAGCAAGACTCTGACAAAGCCCAAGAAATCCAC
AGCGGCGGCAGCAGCAGGAGGAGGAGCGCTGCGTCTGGCGCCCAACGAACCCGTATCGACCCGC
GAGCTTAGAAACAGGATTTTTCCCACTCTGTATGCTATATTTCAACAGAGCAGGGGCCAAGAACAA
GAGCTGAAAATAAAAAACAGGTCTCTGCGATCCCTCACCCGCAGCTGCCTGTATCACAAAAGCGA
AGATCAGCTTCGGCGCACGCTGGAAGACGCGGAGGCTCTCTTCAGTAAATACTGCGCGCTGACTCT
TAAGGACTAGTTTCGCGCCCTTTCTCAAATTTAAGCGCGAAAACTACGTCATCTCCAGCGGCCACA
CCCGGCGCCAGCACCTGTTGTCAGCGCCATTATGAGCAAGGAAATTCCCACGCCCTACATGTGGAG
TTACCAGCCACAAATGGGACTTGCGGCTGGAGCTGCCCAAGACTACTCAACCCGAATAAACTACA
TGAGCGCGGGACCCCACATGATATCCCGGGTCAACGGAATACGCGCCCACCGAAACCGAATTCTC
CTGGAACAGGCGGCTATTACCACCACACCTCGTAATAACCTTAATCCCCGTAGTTGGCCCGCTGCC
CTGGTGTACCAGGAAAGTCCCGCTCCCACCACTGTGGTACTTCCCAGAGACGCCCAGGCCGAAGTT
CAGATGACTAACTCAGGGGCGCAGCTTGCGGGCGGCTTTCGTCACAGGGTGCGGTCGCCCGGGCA
GGGTATAACTCACCTGACAATCAGAGGGCGAGGTATTCAGCTCAACGACGAGTCGGTGAGCTCCT
CGCTTGGTCTCCGTCCGGACGGGACATTTCAGATCGGCGGCGCCGGCCGCTCTTCATTCACGCCTC
GTCAGGCAATCCTAACTCTGCAGACCTCGTCCTCTGAGCCGCGCTCTGGAGGCATTGGAACTCTGC
AATTTATTGAGGAGTTTGTGCCATCGGTCTACTTTAACCCCTTCTCGGGACCTCCCGGCCACTATCC
GGATCAATTTATTCCTAACTTTGACGCGGTAAAGGACTCGGCGGACGGCTACGACTGAATGTTAAG
TGGAGAGGCAGAGCAACTGCGCCTGAAACACCTGGTCCACTGTCGCCGCCACAAGTGCTTTGCCC
GCGACTCCGGTGAGTTTTGCTACTTTGAATTGCCCGAGGATCATATCGAGGGCCCGGCGCACGGCG
TCCGGCTTACCGCCCAGGGAGAGCTTGCCCGTAGCCTGATTCGGGAGTTTACCCAGCGCCCCCTGC
TAGTTGAGCGGGACAGGGGACCCTGTGTTCTCACTGTGATTTGCAACTGTCCTAACCCTGGATTAC
ATCAAGATCTTTGTTGCCATCTCTGTGCTGAGTATAATAAATACAGAAATTAAAATATACTGGGGC
TCCTATCGCCATCCTGTAAACGCCACCGTCTTCACCCGCCCAAGCAAACCAAGGCGAACCTTACCT
GGTACTTTTAACATCTCTCCCTCTGTGATTTACAACAGTTTCAACCCAGACGGAGTGAGTCTACGA
GAGAACCTCTCCGAGCTCAGCTACTCCATCAGAAAAAACACCACCCTCCTTACCTGCCGGGAACGT
ACGAGTGCGTCACCGGCCGCTGCACCACACCTACCGCCTGACCGTAAACCAGACTTTTTCCGGACA
GACCTCAATAACTCTGTTTACCAGAACAGGAGGTGAGCTTAGAAAACCCTTAGGGTATTAGGCCA
AAGGCGCAGCTACTGTGGGGTTTATGAACAATTCAAGCAACTCTACGGGCTATTCTAATTCAGGTT
TCTCTAGAAATGGACGGAATTATTACAGAGCAGCGCCTGCTAGAAAGACGCAGGGCAGCGGCCGA
GCAACAGCGCATGAATCAAGAGCTCCAAGACATGGTTAACTTGCACCAGTGCAAAAGGGGTATCT
TTTGTCTGGTAAAGCAGGCCAAAGTCACCTACGACAGTAATACCACCGGACACCGCCTTAGCTACA
AGTTGCCAACCAAGCGTCAGAAATTGGTGGTCATGGTGGGAGAAAAGCCCATTACCATAACTCAG
CACTCGGTAGAAACCGAAGGCTGCATTCACTCACCTTGTCAAGGACCTGAGGATCTCTGCACCCTT
ATTAAGACCCTGTGCGGTCTCAAAGATCTTATTCCCTTTAACTAATAAAAAAAAATAATAAAGCAT
CACTTACTTAAAATCAGTTAGCAAATTTCTGTCCAGTTTATTCAGCAGCACCTCCTTGCCCTCCTCC
CAGCTCTGGTATTGCAGCTTCCTCCTGGCTGCAAACTTTCTCCACAATCTAAATGGAATGTCAGTTT
CCTCCTGTTCCTGTCCATCCGCACCCACTATCTTCATGTTGTTGCAGATGAAGCGCGCAAGACCGTC
TGAAGATACCTTCAACCCCGTGTATCCATATGACACGGAAACCGGTCCTCCAACTGTGCCTTTTCTT
ACTCCTCCCTTTGTATCCCCCAATGGGTTTCAAGAGAGTCCCCCTGGGGTACTCTCTTTGCGCCTAT
CCGAACCTCTAGTTACCTCCAATGGCATGCTTGCGCTCAAAATGGGCAACGGCCTCTCTCTGGACG
AGGCCGGCAACCTTACCTCCCAAAATGTAACCACTGTGAGCCCACCTCTCAAAAAAACCAAGTCA
AACATAAACCTGGAAATATCTGCACCCCTCACAGTTACCTCAGAAGCCCTAACTGTGGCTGCCGCC
GCACCTCTAATGGTCGCGGGCAACACACTCACCATGCAATCACAGGCCCCGCTAACCGTGCACGA
CTCCAAACTTAGCATTGCCACCCAAGGACCCCTCACAGTGTCAGAAGGAAAGCTAGCCCTGCAAA
CATCAGGCCCCCTCACCACCACCGATAGCAGTACCCTTACTATCACTGCCTCACCCCCTCTAACTA
CTGCCACTGGTAGCTTGGGCATTGACTTGAAAGAGCCCATTTATACACAAAATGGAAAACTAGGA
CTAAAGTACGGGGCTCCTTTGCATGTAACAGACGACCTAAACACTTTGACCGTAGCAACTGGTCCA
GGTGTGACTATTAATAATACTTCCTTGCAAACTAAAGTTACTGGAGCCTTGGGTTTTGATTCACAA
GGCAATATGCAACTTAATGTAGCAGGAGGACTAAGGATTGATTCTCAAAACAGACGCCTTATACTT
GATGTTAGTTATCCGTTTGATGCTCAAAACCAACTAAATCTAAGACTAGGACAGGGCCCTCTTTTT
ATAAACTCAGCCCACAACTTGGATATTAACTACAACAAAGGCCTTTACTTGTTTACAGCTTCAAAC
AATTCCAAAAAGCTTGAGGTTAACCTAAGCACTGCCAAGGGGTTGATGTTTGACGCTACAGCCATA
GCCATTAATGCAGGAGATGGGCTTGAATTTGGTTCACCTAATGCACCAAACACAAATCCCCTCAAA
ACAAAAATTGGCCATGGCCTAGAATTTGATTCAAACAAGGCTATGGTTCCTAAACTAGGAACTGG
CCTTAGTTTTGACAGCACAGGTGCCATTACAGTAGGAAACAAAAATAATGATAAGCTAACTTTGTG
GACCACACCAGCTCCATCTCCTAACTGTAGACTAAATGCAGAGAAAGATGCTAAACTCACTTTGGT
CTTAACAAAATGTGGCAGTCAAATACTTGCTACAGTTTCAGTTTTGGCTGTTAAAGGCAGTTTGGC
TCCAATATCTGGAACAGTTCAAAGTGCTCATCTTATTATAAGATTTGACGAAAATGGAGTGCTACT
AAACAATTCCTTCCTGGACCCAGAATATTGGAACTTTAGAAATGGAGATCTTACTGAAGGCACAGC
CTATACAAACGCTGTTGGATTTATGCCTAACCTATCAGCTTATCCAAAATCTCACGGTAAAACTGC
CAAAAGTAACATTGTCAGTCAAGTTTACTTAAACGGAGACAAAACTAAACCTGTAACACTAACCA
TTACACTAAACGGTACACAGGAAACAGGAGACACAACTCCAAGTGCATACTCTATGTCATTTTCAT
GGGACTGGTCTGGCCACAACTACATTAATGAAATATTTGCCACATCCTCTTACACTTTTTCATACAT
TGCCCAAGAATAAAGAATCGTTTGTGTTATGTTTCAACGTGTTTATTTTTCAATTGCAGAAAATTTC
GAATCATTTTTCATTCAGTAGTATAGCCCCACCACCACATAGCTTATACAGATCACCGTACCTTAAT
CAAACTCACAGAACCCTAGTATTCAACCTGCCACCTCCCTCCCAACACACAGAGTACACAGTCCTT
TCTCCCCGGCTGGCCTTAAAAAGCATCATATCATGGGTAACAGACATATTCTTAGGTGTTATATTC
CACACGGTTTCCTGTCGAGCCAAACGCTCATCAGTGATATTAATAAACTCCCCGGGCAGCTCACTT
AAGTTCATGTCGCTGTCCAGCTGCTGAGCCACAGGCTGCTGTCCAACTTGCGGTTGCTTAACGGGC
GGCGAAGGAGAAGTCCACGCCTACATGGGGGTAGAGTCATAATCGTGCATCAGGATAGGGCGGTG
GTGCTGCAGCAGCGCGCGAATAAACTGCTGCCGCCGCCGCTCCGTCCTGCAGGAATACAACATGG
CAGTGGTCTCCTCAGCGATGATTCGCACCGCCCGCAGCATAAGGCGCCTTGTCCTCCGGGCACAGC
AGCGCACCCTGATCTCACTTAAATCAGCACAGTAACTGCAGCACAGCACCACAATATTGTTCAAAA
TCCCACAGTGCAAGGCGCTGTATCCAAAGCTCATGGCGGGGACCACAGAACCCACGTGGCCATCA
TACCACAAGCGCAGGTAGATTAAGTGGCGACCCCTCATAAACACGCTGGACATAAACATTACCTC
TTTTGGCATGTTGTAATTCACCACCTCCCGGTACCATATAAACCTCTGATTAAACATGGCGCCATCC
ACCACCATCCTAAACCAGCTGGCCAAAACCTGCCCGCCGGCTATACACTGCAGGGAACCGGGACT
GGAACAATGACAGTGGAGAGCCCAGGACTCGTAACCATGGATCATCATGCTCGTCATGATATCAA
TGTTGGCACAACACAGGCACACGTGCATACACTTCCTCAGGATTACAAGCTCCTCCCGCGTTAGAA
CCATATCCCAGGGAACAACCCATTCCTGAATCAGCGTAAATCCCACACTGCAGGGAAGACCTCGC
ACGTAACTCACGTTGTGCATTGTCAAAGTGTTACATTCGGGCAGCAGCGGATGATCCTCCAGTATG
GTAGCGCGGGTTTCTGTCTCAAAAGGAGGTAGACGATCCCTACTGTACGGAGTGCGCCGAGACAA
CCGAGATCGTGTTGGTCGTAGTGTCATGCCAAATGGAACGCCGGACGTAGTCATATTTCCTGAAGC
AAAACCAGGTGCGGGCGTGACAAACAGATCTGCGTCTCCGGTCTCGCCGCTTAGATCGCTCTGTGT
AGTAGTTGTAGTATATCCACTCTCTCAAAGCATCCAGGCGCCCCCTGGCTTCGGGTTCTATGTAAA
CTCCTTCATGCGCCGCTGCCCTGATAACATCCACCACCGCAGAATAAGCCACACCCAGCCAACCTA
CACATTCGTTCTGCGAGTCACACACGGGAGGAGCGGGAAGAGCTGGAAGAACCATGTTTTTTTTTT
TATTCCAAAAGATTATCCAAAACCTCAAAATGAAGATCTATTAAGTGAACGCGCTCCCCTCCGGTG
GCGTGGTCAAACTCTACAGCCAAAGAACAGATAATGGCATTTGTAAGATGTTGCACAATGGCTTCC
AAAAGGCAAACGGCCCTCACGTCCAAGTGGACGTAAAGGCTAAACCCTTCAGGGTGAATCTCCTC
TATAAACATTCCAGCACCTTCAACCATGCCCAAATAATTCTCATCTCGCCACCTTCTCAATATATCT
CTAAGCAAATCCCGAATATTAAGTCCGGCCATTGTAAAAATCTGCTCCAGAGCGCCCTCCACCTTC
AGCCTCAAGCAGCGAATCATGATTGCAAAAATTCAGGTTCCTCACAGACCTGTATAAGATTCAAA
AGCGGAACATTAACAAAAATACCGCGATCCCGTAGGTCCCTTCGCAGGGCCAGCTGAACATAATC
GTGCAGGTCTGCACGGACCAGCGCGGCCACTTCCCCGCCAGGAACCATGACAAAAGAACCCACAC
TGATTATGACACGCATACTCGGAGCTATGCTAACCAGCGTAGCCCCGATGTAAGCTTGTTGCATGG
GCGGCGATATAAAATGCAAGGTGCTGCTCAAAAAATCAGGCAAAGCCTCGCGCAAAAAAGAAAG
CACATCGTAGTCATGCTCATGCAGATAAAGGCAGGTAAGCTCCGGAACCACCACAGAAAAAGACA
CCATTTTTCTCTCAAACATGTCTGCGGGTTTCTGCATAAACACAAAATAAAATAACAAAAAAACAT
TTAAACATTAGAAGCCTGTCTTACAACAGGAAAAACAACCCTTATAAGCATAAGACGGACTACGG
CCATGCCGGCGTGACCGTAAAAAAACTGGTCACCGTGATTAAAAAGCACCACCGACAGCTCCTCG
GTCATGTCCGGAGTCATAATGTAAGACTCGGTAAACACATCAGGTTGATTCACATCGGTCAGTGCT
AAAAAGCGACCGAAATAGCCCGGGGGAATACATACCCGCAGGCGTAGAGACAACATTACAGCCC
CCATAGGAGGTATAACAAAATTAATAGGAGAGAAAAACACATAAACACCTGAAAAACCCTCCTGC
CTAGGCAAAATAGCACCCTCCCGCTCCAGAACAACATACAGCGCTTCCACAGCGGCAGCCATAAC
AGTCAGCCTTACCAGTAAAAAAGAAAACCTATTAAAAAAACACCACTCGACACGGCACCAGCTCA
ATCAGTCACAGTGTAAAAAAGGGCCAAGTGCAGAGCGAGTATATATAGGACTAAAAAATGACGTA
ACGGTTAAAGTCCACAAAAAACACCCAGAAAACCGCACGCGAACCTACGCCCAGAAACGAAAGC
CAAAAAACCCACAACTTCCTCAAATCGTCACTTCCGTTTTCCCACGTTACGTCACTTCCCATTTTAA
GAAAACTACAATTCCCAACACATACAAGTTACTCCGCCCTAAAACCTACGTCACCCGCCCCGTTCC
CACGCCCCGCGCCACGTCACAAACTCCACCCCCTCATTATCATATTGGCTTCAATCCAAAATAAGG
TATATTATTGATGATGTTAATTAATTTAAATCCGCATGCGATATCGAGCTCTCCCGGGAATTCGGAT
CTGCGACGCGAGGCTGGATGGCCTTCCCCATTATGATTCTTCTCGCGTTTAAGGGCACCAATAACT
GCCTTAAAAAAATTACGCCCCGCCCTGCCACTCATCGCAGTACTGTTGTAATTCATTAAGCATTCT
GCCGACATGGAAGCCATCACAAACGGCATGATGAACCTGAATCGCCAGCGGCATCAGCACCTTGT
CGCCTTGCGTATAATATTTGCCCATGGTGAAAACGGGGGCGAAGAAGTTGTCCATATTGGCCACGT
TTAAATCAAAACTGGTGAAACTCACCCAGGGATTGGCTGAGACGAAAAACATATTCTCAATAAAC
CCTTTAGGGAAATAGGCCAGGTTTTCACCGTAACACGCCACATCTTGCGAATATATGTGTAGAAAC
TGCCGGAAATCGTCGTGGTATTCACTCCAGAGCGATGAAAACGTTTCAGTTTGCTCATGGAAAACG
GTGTAACAAGGGTGAACACTATCCCATATCACCAGCTCACCGTCTTTCATTGCCATACGGAATTCC
GGATGAGCATTCATCAGGCGGGCAAGAATGTGAATAAAGGCCGGATAAAACTTGTGCTTATTTTTC
TTTACGGTCTTTAAAAAGGCCGTAATATCCAGCTGAACGGTCTGGTTATAGGTACATTGAGCAACT
GACTGAAATGCCTCAAAATGTTCTTTACGATGCCATTGGGATATATCAACGGTGGTATATCCAGTG
ATTTTTTTCTCCATTTTAGCTTCCTTAGCTCCTGAAAATCTCGATAACTCAAAAAATACGCCCGGTA
GTGATCTTATTTCATTATGGTGAAAGTTGGAACCTCTTACGTGCCGATCAACGTCTCATTTTCGCCA
AAAGTTGGCCCAGGGCTTCCCGGTATCAACAGGGACACCAGGATTTATTTATTCTGCGAAGTGATC
TTCCGTCACAGGTATTTATTCGCGATAAGCTCATGGAGCGGCGTAACCGTCGCACAGGAAGGACA
GAGAAAGCGCGGATCTGGGAAGTGACGGACAGAACGGTCAGGACCTGGATTGGGGAGGCGGTTG
CCGCCGCTGCTGCTGACGGTGTGACGTTCTCTGTTCCGGTCACACCACATACGTTCCGCCATTCCTA
TGCGATGCACATGCTGTATGCCGGTATACCGCTGAAAGTTCTGCAAAGCCTGATGGGACATAAGTC
CATCAGTTCAACGGAAGTCTACACGAAGGTTTTTGCGCTGGATGTGGCTGCCCGGCACCGGGTGCA
GTTTGCGATGCCGGAGTCTGATGCGGTTGCGATGCTGAAACAATTATCCTGAGAATAAATGCCTTG
GCCTTTATATGGAAATGTGGAACTGAGTGGATATGCTGTTTTTGTCTGTTAAACAGAGAAGCTGGC
TGTTATCCACTGAGAAGCGAACGAAACAGTCGGGAAAATCTCCCATTATCGTAGAGATCCGCATT
ATTAATCTCAGGAGCCTGTGTAGCGTTTATAGGAAGTAGTGTTCTGTCATGATGCCTGCAAGCGGT
AACGAAAACGATTTGAATATGCCTTCAGGAACAATAGAAATCTTCGTGCGGTGTTACGTTGAAGTG
GAGCGGATTATGTCAGCAATGGACAGAACAACCTAATGAACACAGAACCATGATGTGGTCTGTCC
TTTTACAGCCAGTAGTGCTCGCCGCAGTCGAGCGACAGGGCGAAGCCCTCGAGTGAGCGAGGAAG
CACCAGGGAACAGCACTTATATATTCTGCTTACACACGATGCCTGAAAAAACTTCCCTTGGGGTTA
TCCACTTATCCACGGGGATATTTTTATAATTATTTTTTTTATAGTTTTTAGATCTTCTTTTTTAGAGC
GCCTTGTAGGCCTTTATCCATGCTGGTTCTAGAGAAGGTGTTGTGACAAATTGCCCTTTCAGTGTGA
CAAATCACCCTCAAATGACAGTCCTGTCTGTGACAAATTGCCCTTAACCCTGTGACAAATTGCCCT
CAGAAGAAGCTGTTTTTTCACAAAGTTATCCCTGCTTATTGACTCTTTTTTATTTAGTGTGACAATC
TAAAAACTTGTCACACTTCACATGGATCTGTCATGGCGGAAACAGCGGTTATCAATCACAAGAAA
CGTAAAAATAGCCCGCGAATCGTCCAGTCAAACGACCTCACTGAGGCGGCATATAGTCTCTCCCG
GGATCAAAAACGTATGCTGTATCTGTTCGTTGACCAGATCAGAAAATCTGATGGCACCCTACAGGA
ACATGACGGTATCTGCGAGATCCATGTTGCTAAATATGCTGAAATATTCGGATTGACCTCTGCGGA
AGCCAGTAAGGATATACGGCAGGCATTGAAGAGTTTCGCGGGGAAGGAAGTGGTTTTTTATCGCC
CTGAAGAGGATGCCGGCGATGAAAAAGGCTATGAATCTTTTCCTTGGTTTATCAAACGTGCGCACA
GTCCATCCAGAGGGCTTTACAGTGTACATATCAACCCATATCTCATTCCCTTCTTTATCGGGTTACA
GAACCGGTTTACGCAGTTTCGGCTTAGTGAAACAAAAGAAATCACCAATCCGTATGCCATGCGTTT
ATACGAATCCCTGTGTCAGTATCGTAAGCCGGATGGCTCAGGCATCGTCTCTCTGAAAATCGACTG
GATCATAGAGCGTTACCAGCTGCCTCAAAGTTACCAGCGTATGCCTGACTTCCGCCGCCGCTTCCT
GCAGGTCTGTGTTAATGAGATCAACAGCAGAACTCCAATGCGCCTCTCATACATTGAGAAAAAGA
AAGGCCGCCAGACGACTCATATCGTATTTTCCTTCCGCGATATCACTTCCATGACGACAGGATAGT
CTGAGGGTTATCTGTCACAGATTTGAGGGTGGTTCGTCACATTTGTTCTGACCTACTGAGGGTAATT
TGTCACAGTTTTGCTGTTTCCTTCAGCCTGCATGGATTTTCTCATACTTTTTGAACTGTAATTTTTAA
GGAAGCCAAATTTGAGGGCAGTTTGTCACAGTTGATTTCCTTCTCTTTCCCTTCGTCATGTGACCTG
ATATCGGGGGTTAGTTCGTCATCATTGATGAGGGTTGATTATCACAGTTTATTACTCTGAATTGGCT
ATCCGCGTGTGTACCTCTACCTGGAGTTTTTCCCACGGTGGATATTTCTTCTTGCGCTGAGCGTAAG
AGCTATCTGACAGAACAGTTCTTCTTTGCTTCCTCGCCAGTTCGCTCGCTATGCTCGGTTACACGGC
TGCGGCGAGCGCTAGTGATAATAAGTGACTGAGGTATGTGCTCTTCTTATCTCCTTTTGTAGTGTTG
CTCTTATTTTAAACAACTTTGCGGTTTTTTGATGACTTTGCGATTTTGTTGTTGCTTTGCAGTAAATT
GCAAGATTTAATAAAAAAACGCAAAGCAATGATTAAAGGATGTTCAGAATGAAACTCATGGAAAC
ACTTAACCAGTGCATAAACGCTGGTCATGAAATGACGAAGGCTATCGCCATTGCACAGTTTAATGA
TGACAGCCCGGAAGCGAGGAAAATAACCCGGCGCTGGAGAATAGGTGAAGCAGCGGATTTAGTT
GGGGTTTCTTCTCAGGCTATCAGAGATGCCGAGAAAGCAGGGCGACTACCGCACCCGGATATGGA
AATTCGAGGACGGGTTGAGCAACGTGTTGGTTATACAATTGAACAAATTAATCATATGCGTGATGT
GTTTGGTACGCGATTGCGACGTGCTGAAGACGTATTTCCACCGGTGATCGGGGTTGCTGCCCATAA
AGGTGGCGTTTACAAAACCTCAGTTTCTGTTCATCTTGCTCAGGATCTGGCTCTGAAGGGGCTACG
TGTTTTGCTCGTGGAAGGTAACGACCCCCAGGGAACAGCCTCAATGTATCACGGATGGGTACCAG
ATCTTCATATTCATGCAGAAGACACTCTCCTGCCTTTCTATCTTGGGGAAAAGGACGATGTCACTT
ATGCAATAAAGCCCACTTGCTGGCCGGGGCTTGACATTATTCCTTCCTGTCTGGCTCTGCACCGTAT
TGAAACTGAGTTAATGGGCAAATTTGATGAAGGTAAACTGCCCACCGATCCACACCTGATGCTCCG
ACTGGCCATTGAAACTGTTGCTCATGACTATGATGTCATAGTTATTGACAGCGCGCCTAACCTGGG
TATCGGCACGATTAATGTCGTATGTGCTGCTGATGTGCTGATTGTTCCCACGCCTGCTGAGTTGTTT
GACTACACCTCCGCACTGCAGTTTTTCGATATGCTTCGTGATCTGCTCAAGAACGTTGATCTTAAAG
GGTTCGAGCCTGATGTACGTATTTTGCTTACCAAATACAGCAATAGTAATGGCTCTCAGTCCCCGT
GGATGGAGGAGCAAATTCGGGATGCCTGGGGAAGCATGGTTCTAAAAAATGTTGTACGTGAAACG
GATGAAGTTGGTAAAGGTCAGATCCGGATGAGAACTGTTTTTGAACAGGCCATTGATCAACGCTCT
TCAACTGGTGCCTGGAGAAATGCTCTTTCTATTTGGGAACCTGTCTGCAATGAAATTTTCGATCGTC
TGATTAAACCACGCTGGGAGATTAGATAATGAAGCGTGCGCCTGTTATTCCAAAACATACGCTCAA
TACTCAACCGGTTGAAGATACTTCGTTATCGACACCAGCTGCCCCGATGGTGGATTCGTTAATTGC
GCGCGTAGGAGTAATGGCTCGCGGTAATGCCATTACTTTGCCTGTATGTGGTCGGGATGTGAAGTT
TACTCTTGAAGTGCTCCGGGGTGATAGTGTTGAGAAGACCTCTCGGGTATGGTCAGGTAATGAACG
TGACCAGGAGCTGCTTACTGAGGACGCACTGGATGATCTCATCCCTTCTTTTCTACTGACTGGTCA
ACAGACACCGGCGTTCGGTCGAAGAGTATCTGGTGTCATAGAAATTGCCGATGGGAGTCGCCGTC
GTAAAGCTGCTGCACTTACCGAAAGTGATTATCGTGTTCTGGTTGGCGAGCTGGATGATGAGCAGA
TGGCTGCATTATCCAGATTGGGTAACGATTATCGCCCAACAAGTGCTTATGAACGTGGTCAGCGTT
ATGCAAGCCGATTGCAGAATGAATTTGCTGGAAATATTTCTGCGCTGGCTGATGCGGAAAATATTT
CACGTAAGATTATTACCCGCTGTATCAACACCGCCAAATTGCCTAAATCAGTTGTTGCTCTTTTTTC
TCACCCCGGTGAACTATCTGCCCGGTCAGGTGATGCACTTCAAAAAGCCTTTACAGATAAAGAGG
AATTACTTAAGCAGCAGGCATCTAACCTTCATGAGCAGAAAAAAGCTGGGGTGATATTTGAAGCT
GAAGAAGTTATCACTCTTTTAACTTCTGTGCTTAAAACGTCATCTGCATCAAGAACTAGTTTAAGCT
CACGACATCAGTTTGCTCCTGGAGCGACAGTATTGTATAAGGGCGATAAAATGGTGCTTAACCTGG
ACAGGTCTCGTGTTCCAACTGAGTGTATAGAGAAAATTGAGGCCATTCTTAAGGAACTTGAAAAG
CCAGCACCCTGATGCGACCACGTTTTAGTCTACGTTTATCTGTCTTTACTTAATGTCCTTTGTTACA
GGCCAGAAAGCATAACTGGCCTGAATATTCTCTCTGGGCCCACTGTTCCACTTGTATCGTCGGTCT
GATAATCAGACTGGGACCACGGTCCCACTCGTATCGTCGGTCTGATTATTAGTCTGGGACCACGGT
CCCACTCGTATCGTCGGTCTGATTATTAGTCTGGGACCACGGTCCCACTCGTATCGTCGGTCTGATA
ATCAGACTGGGACCACGGTCCCACTCGTATCGTCGGTCTGATTATTAGTCTGGGACCATGGTCCCA
CTCGTATCGTCGGTCTGATTATTAGTCTGGGACCACGGTCCCACTCGTATCGTCGGTCTGATTATTA
GTCTGGAACCACGGTCCCACTCGTATCGTCGGTCTGATTATTAGTCTGGGACCACGGTCCCACTCG
TATCGTCGGTCTGATTATTAGTCTGGGACCACGATCCCACTCGTGTTGTCGGTCTGATTATCGGTCT
GGGACCACGGTCCCACTTGTATTGTCGATCAGACTATCAGCGTGAGACTACGATTCCATCAATGCC
TGTCAAGGGCAAGTATTGACATGTCGTCGTAACCTGTAGAACGGAGTAACCTCGGTGTGCGGTTGT
ATGCCTGCTGTGGATTGCTGCTGTGTCCTGCTTATCCACAACATTTTGCGCACGGTTATGTGGACAA
AATACCTGGTTACCCAGGCCGTGCCGGCACGTTAACCGGGCACATTTCCCCGAAAAGTGCCACCTG
ACGTCTAAGAAACCATTATTATCATGACATTAACCTATAAAAATAGGCGTATCACGAGGCCCTTTC
GTCTTCAAGAATTGGATCCGAATTCCCGGGAGAGCTCGATATCGCATGCGGATTTAAATTAATTAA
* C1B
(SEQ ID NO: 83)
CATCATCAATAATATACCTTATTTTGGATTGAAGCCAATATGATAATGAGGGGGTGGAGTTTGTGA
CGTGGCGCGGGGCGTGGGAACGGGGCGGGTGACGTAGTAGTGTGGCGGAAGTGTGATGTTGCAAG
TGTGGCGGAACACATGTAAGCGACGGATGTGGCAAAAGTGACGTTTTTGGTGTGCGCCGGTGTAC
ACAGGAAGTGACAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAATTTTGTGTTACT
CATAGCGCGTAATATTTGTCTAGGGCCGCGGGGACTTTGACCGTTTACGTGGAGACTCGCCCAGGT
GTTTTTCTCAGGTGTTTTCCGCGTTCCGGGTCAAAGTTGGCGTTTTATTATTATAGTCAGTCGAAGC
TTGGATCCGGTACCTCTAGAATTCTCGAGCGGCCGCTAGCGACATCGGATCTCCCGATCCCCTATG
GTGCACTCTCAGTACAATCTGCTCTGATGCCGCATAGTTAAGCCAGTATCTGCTCCCTGCTTGTGTG
TTGGAGGTCGCTGAGTAGTGCGCGAGCAAAATTTAAGCTACAACAAGGCAAGGCTTGACCGACAA
TTGCATGAAGAATCTGCTTAGGGTTAGGCGTTTTGCGCTGCTTCGCGATGTACGGGCCAGATATAC
GCGTTGACATTGATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACG
TCAATGGGTGGACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCTCTG
GCTAACTAGAGAACCCACTGCTTACTGGCTTATCGAAATGAGACCCAAGCTGGCTAGTTAAGCTAT
CAACAAGTTTGTACAAAAAAGCAGGCTTTAAAGGAACCAATTCAGTCGACTGGATCCGGTACCAC
CATGTTCCTGAACTGCTGCCCAGGTTGCTGTATGGAGCCTGAATTCACCATGGTGAGCAAGGGCGA
GGAGCTGTTCACCGGGGTGGTGCCCATCCTGGTCGAGCTGGACGGCGACGTAAACGGCCACAAGT
TCAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTGGGGCGTGCAGTGC
TTCGCCCGCTACCCCGACCACATGAAGCAGCACGACTTCTTCAAGTCCGCCATGCCCGAAGGCTAC
GTCCAGGAGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGACGGCAAC
ATCCTGGGGCACAAGCTGGAGTACAACGCCATCAGCGACAACGTCTATATCACCGCCGACAAGCA
GAAGAACGGCATCAAGGCCAACTTCAAGATCCGCCACAACATCGAGGACGGCAGCGTGCAGCTCG
CCGACCACTACCAGCAGAACACCCCCATCGGCGACGGCCCCGTGCTGCTGCCCGACAACCACTAC
CTGAGCACCCAGTCCAAGCTGAGCAAAGACCCCAACGAGAAGCGCGATCACATGGTCCTGCTGGA
GTTCGTGACCGCCGCCGGGATCACTCTCGGCATGGACGAGCTGTACAAGGTCGACTATCCGTACGA
CGTACCAGACTACGCATAACCGCGGCCGCACTCGAGATATCTAGACCCAGCTTTCTTGTACAAAGT
GGTTGATCTAGAGGGCCCGCGGTTCGAAGGTAAGCCTATCCCTAACCCTCTCCTCGGTCTCGATTC
TACGCGTACCGGTTAGTAATGAGTTTAAACGGGGGAGGCTAACTGAAACACGGAAGGAGACAATA
CCGGAAGGAACCCGCGCTATGACGGCAATAAAAAGACAGAATAAAACGCACGGGTGTTGGGTCG
TTTGTTCATAAACGCGGGGTTCGGTCCCAGGGCTGGCACTCTGTCGATACCCCACCGAGACCCCAT
TGGGGCCAATACGCCCGCGTTTCTTCCTTTTCCCCACCCCACCCCCCAAGTTCGGGTGAAGGCCCA
GGGCTCGCAGCCAACGTCGGGGCGGCAGGCCCTGCCATAGCAGATCCGATTCGACAGATCACTGA
AATGTGTGGGCGTGGCTTAAGGGTGGGAAAGAATATATAAGGTGGGGGTCTTATGTAGTTTTGTAT
CTGTTTTGCAGCAGCCGCCGCCGCCATGAGCACCAACTCGTTTGATGGAAGCATTGTGAGCTCATA
TTTGACAACGCGCATGCCCCCATGGGCCGGGGTGCGTCAGAATGTGATGGGCTCCAGCATTGATG
GTCGCCCCGTCCTGCCCGCAAACTCTACTACCTTGACCTACGAGACCGTGTCTGGAACGCCGTTGG
AGACTGCAGCCTCCGCCGCCGCTTCAGCCGCTGCAGCCACCGCCCGCGGGATTGTGACTGACTTTG
CTTTCCTGAGCCCGCTTGCAAGCAGTGCAGCTTCCCGTTCATCCGCCCGCGATGACAAGTTGACGG
CTCTTTTGGCACAATTGGATTCTTTGACCCGGGAACTTAATGTCGTTTCTCAGCAGCTGTTGGATCT
GCGCCAGCAGGTTTCTGCCCTGAAGGCTTCCTCCCCTCCCAATGCGGTTTAAAACATAAATAAAAA
ACCAGACTCTGTTTGGATTTGGATCAAGCAAGTGTCTTGCTGTCTTTATTTAGGGGTTTTGCGCGCG
CGGTAGGCCCGGGACCAGCGGTCTCGGTCGTTGAGGGTCCTGTGTATTTTTTCCAGGACGTGGTAA
AGGTGACTCTGGATGTTCAGATACATGGGCATAAGCCCGTCTCTGGGGTGGAGGTAGCACCACTG
CAGAGCTTCATGCTGCGGGGTGGTGTTGTAGATGATCCAGTCGTAGCAGGAGCGCTGGGCGTGGT
GCCTAAAAATGTCTTTCAGTAGCAAGCTGATTGCCAGGGGCAGGCCCTTGGTGTAAGTGTTTACAA
AGCGGTTAAGCTGGGATGGGTGCATACGTGGGGATATGAGATGCATCTTGGACTGTATTTTTAGGT
TGGCTATGTTCCCAGCCATATCCCTCCGGGGATTCATGTTGTGCAGAACCACCAGCACAGTGTATC
CGGTGCACTTGGGAAATTTGTCATGTAGCTTAGAAGGAAATGCGTGGAAGAACTTGGAGACGCCC
TTGTGACCTCCAAGATTTTCCATGCATTCGTCCATAATGATGGCAATGGGCCCACGGGCGGCGGCC
TGGGCGAAGATATTTCTGGGATCACTAACGTCATAGTTGTGTTCCAGGATGAGATCGTCATAGGCC
ATTTTTACAAAGCGCGGGCGGAGGGTGCCAGACTGCGGTATAATGGTTCCATCCGGCCCAGGGGC
GTAGTTACCCTCACAGATTTGCATTTCCCACGCTTTGAGTTCAGATGGGGGGATCATGTCTACCTGC
GGGGCGATGAAGAAAACGGTTTCCGGGGTAGGGGAGATCAGCTGGGAAGAAAGCAGGTTCCTGA
GCAGCTGCGACTTACCGCAGCCGGTGGGCCCGTAAATCACACCTATTACCGGCTGCAACTGGTAGT
TAAGAGAGCTGCAGCTGCCGTCATCCCTGAGCAGGGGGGCCACTTCGTTAAGCATGTCCCTGACTC
GCATGTTTTCCCTGACCAAATCCGCCAGAAGGCGCTCGCCGCCCAGCGATAGCAGTTCTTGCAAGG
AAGCAAAGTTTTTCAACGGTTTGAGACCGTCCGCCGTAGGCATGCTTTTGAGCGTTTGACCAAGCA
GTTCCAGGCGGTCCCACAGCTCGGTCACCTGCTCTACGGCATCTCGATCCAGCATATCTCCTCGTTT
CGCGGGTTGGGGCGGCTTTCGCTGTACGGCAGTAGTCGGTGCTCGTCCAGACGGGCCAGGGTCAT
GTCTTTCCACGGGCGCAGGGTCCTCGTCAGCGTAGTCTGGGTCACGGTGAAGGGGTGCGCTCCGGG
CTGCGCGCTGGCCAGGGTGCGCTTGAGGCTGGTCCTGCTGGTGCTGAAGCGCTGCCGGTCTTCGCC
CTGCGCGTCGGCCAGGTAGCATTTGACCATGGTGTCATAGTCCAGCCCCTCCGCGGCGTGGCCCTT
GGCGCGCAGCTTGCCCTTGGAGGAGGCGCCGCACGAGGGGCAGTGCAGACTTTTGAGGGCGTAGA
GCTTGGGCGCGAGAAATACCGATTCCGGGGAGTAGGCATCCGCGCCGCAGGCCCCGCAGACGGTC
TCGCATTCCACGAGCCAGGTGAGCTCTGGCCGTTCGGGGTCAAAAACCAGGTTTCCCCCATGCTTT
TTGATGCGTTTCTTACCTCTGGTTTCCATGAGCCGGTGTCCACGCTCGGTGACGAAAAGGCTGTCC
GTGTCCCCGTATACAGACTTGAGAGGCCTGTCCTCGAGCGGTGTTCCGCGGTCCTCCTCGTATAGA
AACTCGGACCACTCTGAGACAAAGGCTCGCGTCCAGGCCAGCACGAAGGAGGCTAAGTGGGAGG
GGTAGCGGTCGTTGTCCACTAGGGGGTCCACTCGCTCCAGGGTGTGAAGACACATGTCGCCCTCTT
CGGCATCAAGGAAGGTGATTGGTTTGTAGGTGTAGGCCACGTGACCGGGTGTTCCTGAAGGGGGG
CTATAAAAGGGGGTGGGGGCGCGTTCGTCCTCACTCTCTTCCGCATCGCTGTCTGCGAGGGCCAGC
TGTTGGGGTGAGTACTCCCTCTGAAAAGCGGGCATGACTTCTGCGCTAAGATTGTCAGTTTCCAAA
AACGAGGAGGATTTGATATTCACCTGGCCCGCGGTGATGCCTTTGAGGGTGGCCGCATCCATCTGG
TCAGAAAAGACAATCTTTTTGTTGTCAAGCTTGGTGGCAAACGACCCGTAGAGGGCGTTGGACAG
CAACTTGGCGATGGAGCGCAGGGTTTGGTTTTTGTCGCGATCGGCGCGCTCCTTGGCCGCGATGTT
TAGCTGCACGTATTCGCGCGCAACGCACCGCCATTCGGGAAAGACGGTGGTGCGCTCGTCGGGCA
CCAGGTGCACGCGCCAACCGCGGTTGTGCAGGGTGACAAGGTCAACGCTGGTGGCTACCTCTCCG
CGTAGGCGCTCGTTGGTCCAGCAGAGGCGGCCGCCCTTGCGCGAGCAGAATGGCGGTAGGGGGTC
TAGCTGCGTCTCGTCCGGGGGGTCTGCGTCCACGGTAAAGACCCCGGGCAGCAGGCGCGCGTCGA
AGTAGTCTATCTTGCATCCTTGCAAGTCTAGCGCCTGCTGCCATGCGCGGGCGGCAAGCGCGCGCT
CGTATGGGTTGAGTGGGGGACCCCATGGCATGGGGTGGGTGAGCGCGGAGGCGTACATGCCGCAA
ATGTCGTAAACGTAGAGGGGCTCTCTGAGTATTCCAAGATATGTAGGGTAGCATCTTCCACCGCGG
ATGCTGGCGCGCACGTAATCGTATAGTTCGTGCGAGGGAGCGAGGAGGTCGGGACCGAGGTTGCT
ACGGGCGGGCTGCTCTGCTCGGAAGACTATCTGCCTGAAGATGGCATGTGAGTTGGATGATATGGT
TGGACGCTGGAAGACGTTGAAGCTGGCGTCTGTGAGACCTACCGCGTCACGCACGAAGGAGGCGT
AGGAGTCGCGCAGCTTGTTGACCAGCTCGGCGGTGACCTGCACGTCTAGGGCGCAGTAGTCCAGG
GTTTCCTTGATGATGTCATACTTATCCTGTCCCTTTTTTTTCCACAGCTCGCGGTTGAGGACAAACT
CTTCGCGGTCTTTCCAGTACTCTTGGATCGGAAACCCGTCGGCCTCCGAACGGTAAGAGCCTAGCA
TGTAGAACTGGTTGACGGCCTGGTAGGCGCAGCATCCCTTTTCTACGGGTAGCGCGTATGCCTGCG
CGGCCTTCCGGAGCGAGGTGTGGGTGAGCGCAAAGGTGTCCCTGACCATGACCAGCATGAAGGGC
ACGAGCTGCTTCCCAAAGGCCCCCATCCAAGTATAGGTCTCTACATCGTAGGTGACAAAGAGACG
CTCGGTGCGAGGATGCGAGCCGATCGGGAAGAACTGGATCTCCCGCCACCAATTGGAGGAGTGGC
TATTGATGTGGTGAAAGTAGAAGTCCCTGCGACGGGCCGAACACTCGTGCTGGCTTTTGTAAAAAC
GTGCGCAGTACTGGCAGCGGTGCACGGGCTGTACATCCTGCACGAGGTTGACCTGACGACCGCGC
ACAAGGAAGCAGAGTGGGAATTTGAGCCCCTCGCCTGGCGGGTTTGGCTGGTGGTCTTCTACTTCG
GCTGCTTGTCCTTGACCGTCTGGCTGCTCGAGGGGAGTTACGGTGGATCGGACCACCACGCCGCGC
GAGCCCAAAGTCCAGATGTCCGCGCGCGGCGGTCGGAGCTTGATGACAACATCGCGCAGATGGGA
GCTGTCCATGGTCTGGAGCTCCCGCGGCGTCAGGTCAGGCGGGAGCTCCTGCAGGTTTACCTCGCA
TAGACGGGTCAGGGCGCGGGCTAGATCCAGGTGATACCTAATTTCCAGGGGCTGGTTGGTGGCGG
CGTCGATGGCTTGCAAGAGGCCGCATCCCCGCGGCGCGACTACGGTACCGCGCGGCGGGCGGTGG
GCCGCGGGGGTGTCCTTGGATGATGCATCTAAAAGCGGTGACGCGGGCGAGCCCCCGGAGGTAGG
GGGGGCTCCGGACCCGCCGGGAGAGGGGGCAGGGGCACGTCGGCGCCGCGCGCGGGCAGGAGCT
GGTGCTGCGCGCGTAGGTTGCTGGCGAACGCGACGACGCGGCGGTTGATCTCCTGAATCTGGCGC
CTCTGCGTGAAGACGACGGGCCCGGTGAGCTTGAACCTGAAAGAGAGTTCGACAGAATCAATTTC
GGTGTCGTTGACGGCGGCCTGGCGCAAAATCTCCTGCACGTCTCCTGAGTTGTCTTGATAGGCGAT
CTCGGCCATGAACTGCTCGATCTCTTCCTCCTGGAGATCTCCGCGTCCGGCTCGCTCCACGGTGGC
GGCGAGGTCGTTGGAAATGCGGGCCATGAGCTGCGAGAAGGCGTTGAGGCCTCCCTCGTTCCAGA
CGCGGCTGTAGACCACGCCCCCTTCGGCATCGCGGGCGCGCATGACCACCTGCGCGAGATTGAGC
TCCACGTGCCGGGCGAAGACGGCGTAGTTTCGCAGGCGCTGAAAGAGGTAGTTGAGGGTGGTGGC
GGTGTGTTCTGCCACGAAGAAGTACATAACCCAGCGTCGCAACGTGGATTCGTTGATATCCCCCAA
GGCCTCAAGGCGCTCCATGGCCTCGTAGAAGTCCACGGCGAAGTTGAAAAACTGGGAGTTGCGCG
CCGACACGGTTAACTCCTCCTCCAGAAGACGGATGAGCTCGGCGACAGTGTCGCGCACCTCGCGCT
CAAAGGCTACAGGGGCCTCTTCTTCTTCTTCAATCTCCTCTTCCATAAGGGCCTCCCCTTCTTCTTCT
TCTGGCGGCGGTGGGGGAGGGGGGACACGGCGGCGACGACGGCGCACCGGGAGGCGGTCGACAA
AGCGCTCGATCATCTCCCCGCGGCGACGGCGCATGGTCTCGGTGACGGCGCGGCCGTTCTCGCGGG
GGCGCAGTTGGAAGACGCCGCCCGTCATGTCCCGGTTATGGGTTGGCGGGGGGCTGCCATGCGGC
AGGGATACGGCGCTAACGATGCATCTCAACAATTGTTGTGTAGGTACTCCGCCGCCGAGGGACCT
GAGCGAGTCCGCATCGACCGGATCGGAAAACCTCTCGAGAAAGGCGTCTAACCAGTCACAGTCGC
AAGGTAGGCTGAGCACCGTGGCGGGCGGCAGCGGGCGGCGGTCGGGGTTGTTTCTGGCGGAGGTG
CTGCTGATGATGTAATTAAAGTAGGCGGTCTTGAGACGGCGGATGGTCGACAGAAGCACCATGTC
CTTGGGTCCGGCCTGCTGAATGCGCAGGCGGTCGGCCATGCCCCAGGCTTCGTTTTGACATCGGCG
CAGGTCTTTGTAGTAGTCTTGCATGAGCCTTTCTACCGGCACTTCTTCTTCTCCTTCCTCTTGTCCTG
CATCTCTTGCATCTATCGCTGCGGCGGCGGCGGAGTTTGGCCGTAGGTGGCGCCCTCTTCCTCCCAT
GCGTGTGACCCCGAAGCCCCTCATCGGCTGAAGCAGGGCTAGGTCGGCGACAACGCGCTCGGCTA
ATATGGCCTGCTGCACCTGCGTGAGGGTAGACTGGAAGTCATCCATGTCCACAAAGCGGTGGTAT
GCGCCCGTGTTGATGGTGTAAGTGCAGTTGGCCATAACGGACCAGTTAACGGTCTGGTGACCCGGC
TGCGAGAGCTCGGTGTACCTGAGACGCGAGTAAGCCCTCGAGTCAAATACGTAGTCGTTGCAAGT
CCGCACCAGGTACTGGTATCCCACCAAAAAGTGCGGCGGCGGCTGGCGGTAGAGGGGCCAGCGTA
GGGTGGCCGGGGCTCCGGGGGCGAGATCTTCCAACATAAGGCGATGATATCCGTAGATGTACCTG
GACATCCAGGTGATGCCGGCGGCGGTGGTGGAGGCGCGCGGAAAGTCGCGGACGCGGTTCCAGAT
GTTGCGCAGCGGCAAAAAGTGCTCCATGGTCGGGACGCTCTGGCCGGTCAGGCGCGCGCAATCGT
TGACGCTCTAGACCGTGCAAAAGGAGAGCCTGTAAGCGGGCACTCTTCCGTGGTCTGGTGGATAA
ATTCGCAAGGGTATCATGGCGGACGACCGGGGTTCGAGCCCCGTATCCGGCCGTCCGCCGTGATCC
ATGCGGTTACCGCCCGCGTGTCGAACCCAGGTGTGCGACGTCAGACAACGGGGGAGTGCTCCTTTT
GGCTTCCTTCCAGGCGCGGCGGCTGCTGCGCTAGCTTTTTTGGCCACTGGCCGCGCGCAGCGTAAG
CGGTTAGGCTGGAAAGCGAAAGCATTAAGTGGCTCGCTCCCTGTAGCCGGAGGGTTATTTTCCAAG
GGTTGAGTCGCGGGACCCCCGGTTCGAGTCTCGGACCGGCCGGACTGCGGCGAACGGGGGTTTGC
CTCCCCGTCATGCAAGACCCCGCTTGCAAATTCCTCCGGAAACAGGGACGAGCCCCTTTTTTGCTT
TTCCCAGATGCATCCGGTGCTGCGGCAGATGCGCCCCCCTCCTCAGCAGCGGCAAGAGCAAGAGC
AGCGGCAGACATGCAGGGCACCCTCCCCTCCTCCTACCGCGTCAGGAGGGGCGACATCCGCGGTT
GACGCGGCAGCAGATGGTGATTACGAACCCCCGCGGCGCCGGGCCCGGCACTACCTGGACTTGGA
GGAGGGCGAGGGCCTGGCGCGGCTAGGAGCGCCCTCTCCTGAGCGGCACCCAAGGGTGCAGCTGA
AGCGTGATACGCGTGAGGCGTACGTGCCGCGGCAGAACCTGTTTCGCGACCGCGAGGGAGAGGAG
CCCGAGGAGATGCGGGATCGAAAGTTCCACGCAGGGCGCGAGCTGCGGCATGGCCTGAATCGCGA
GCGGTTGCTGCGCGAGGAGGACTTTGAGCCCGACGCGCGAACCGGGATTAGTCCCGCGCGCGCAC
ACGTGGCGGCCGCCGACCTGGTAACCGCATACGAGCAGACGGTGAACCAGGAGATTAACTTTCAA
AAAAGCTTTAACAACCACGTGCGTACGCTTGTGGCGCGCGAGGAGGTGGCTATAGGACTGATGCA
TCTGTGGGACTTTGTAAGCGCGCTGGAGCAAAACCCAAATAGCAAGCCGCTCATGGCGCAGCTGT
TCCTTATAGTGCAGCACAGCAGGGACAACGAGGCATTCAGGGATGCGCTGCTAAACATAGTAGAG
CCCGAGGGCCGCTGGCTGCTCGATTTGATAAACATCCTGCAGAGCATAGTGGTGCAGGAGCGCAG
CTTGAGCCTGGCTGACAAGGTGGCCGCCATCAACTATTCCATGCTTAGCCTGGGCAAGTTTTACGC
CCGCAAGATATACCATACCCCTTACGTTCCCATAGACAAGGAGGTAAAGATCGAGGGGTTCTACA
TGCGCATGGCGCTGAAGGTGCTTACCTTGAGCGACGACCTGGGCGTTTATCGCAACGAGCGCATCC
ACAAGGCCGTGAGCGTGAGCCGGCGGCGCGAGCTCAGCGACCGCGAGCTGATGCACAGCCTGCAA
AGGGCCCTGGCTGGCACGGGCAGCGGCGATAGAGAGGCCGAGTCCTACTTTGACGCGGGCGCTGA
CCTGCGCTGGGCCCCAAGCCGACGCGCCCTGGAGGCAGCTGGGGCCGGACCTGGGCTGGCGGTGG
CACCCGCGCGCGCTGGCAACGTCGGCGGCGTGGAGGAATATGACGAGGACGATGAGTACGAGCC
AGAGGACGGCGAGTACTAAGCGGTGATGTTTCTGATCAGATGATGCAAGACGCAACGGACCCGGC
GGTGCGGGCGGCGCTGCAGAGCCAGCCGTCCGGCCTTAACTCCACGGACGACTGGCGCCAGGTCA
TGGACCGCATCATGTCGCTGACTGCGCGCAATCCTGACGCGTTCCGGCAGCAGCCGCAGGCCAAC
CGGCTCTCCGCAATTCTGGAAGCGGTGGTCCCGGCGCGCGCAAACCCCACGCACGAGAAGGTGCT
GGCGATCGTAAACGCGCTGGCCGAAAACAGGGCCATCCGGCCCGACGAGGCCGGCCTGGTCTACG
ACGCGCTGCTTCAGCGCGTGGCTCGTTACAACAGCGGCAACGTGCAGACCAACCTGGACCGGCTG
GTGGGGGATGTGCGCGAGGCCGTGGCGCAGCGTGAGCGCGCGCAGCAGCAGGGCAACCTGGGCT
CCATGGTTGCACTAAACGCCTTCCTGAGTACACAGCCCGCCAACGTGCCGCGGGGACAGGAGGAC
TACACCAACTTTGTGAGCGCACTGCGGCTAATGGTGACTGAGACACCGCAAAGTGAGGTGTACCA
GTCTGGGCCAGACTATTTTTTCCAGACCAGTAGACAAGGCCTGCAGACCGTAAACCTGAGCCAGG
CTTTCAAAAACTTGCAGGGGCTGTGGGGGGTGCGGGCTCCCACAGGCGACCGCGCGACCGTGTCT
AGCTTGCTGACGCCCAACTCGCGCCTGTTGCTGCTGCTAATAGCGCCCTTCACGGACAGTGGCAGC
GTGTCCCGGGACACATACCTAGGTCACTTGCTGACACTGTACCGCGAGGCCATAGGTCAGGCGCAT
GTGGACGAGCATACTTTCCAGGAGATTACAAGTGTCAGCCGCGCGCTGGGGCAGGAGGACACGGG
CAGCCTGGAGGCAACCCTAAACTACCTGCTGACCAACCGGCGGCAGAAGATCCCCTCGTTGCACA
GTTTAAACAGCGAGGAGGAGCGCATTTTGCGCTACGTGCAGCAGAGCGTGAGCCTTAACCTGATG
CGCGACGGGGTAACGCCCAGCGTGGCGCTGGACATGACCGCGCGCAACATGGAACCGGGCATGTA
TGCCTCAAACCGGCCGTTTATCAACCGCCTAATGGACTACTTGCATCGCGCGGCCGCCGTGAACCC
CGAGTATTTCACCAATGCCATCTTGAACCCGCACTGGCTACCGCCCCCTGGTTTCTACACCGGGGG
ATTCGAGGTGCCCGAGGGTAACGATGGATTCCTCTGGGACGACATAGACGACAGCGTGTTTTCCCC
GCAACCGCAGACCCTGCTAGAGTTGCAACAGCGCGAGCAGGCAGAGGCGGCGCTGCGAAAGGAA
AGCTTCCGCAGGCCAAGCAGCTTGTCCGATCTAGGCGCTGCGGCCCCGCGGTCAGATGCTAGTAGC
CCATTTCCAAGCTTGATAGGGTCTCTTACCAGCACTCGCACCACCCGCCCGCGCCTGCTGGGCGAG
GAGGAGTACCTAAACAACTCGCTGCTGCAGCCGCAGCGCGAAAAAAACCTGCCTCCGGCATTTCC
CAACAACGGGATAGAGAGCCTAGTGGACAAGATGAGTAGATGGAAGACGTACGCGCAGGAGCAC
AGGGACGTGCCAGGCCCGCGCCCGCCCACCCGTCGTCAAAGGCACGACCGTCAGCGGGGTCTGGT
GTGGGAGGACGATGACTCGGCAGACGACAGCAGCGTCCTGGATTTGGGAGGGAGTGGCAACCCGT
TTGCGCACCTTCGCCCCAGGCTGGGGAGAATGTTTTAAAAAAAAAAAAAGCATGATGCAAAATAA
AAAACTCACCAAGGCCATGGCACCGAGCGTTGGTTTTCTTGTATTCCCCTTAGTATGCGGCGCGCG
GCGATGTATGAGGAAGGTCCTCCTCCCTCCTACGAGAGTGTGGTGAGCGCGGCGCCAGTGGCGGC
GGCGCTGGGTTCTCCCTTCGATGCTCCCCTGGACCCGCCGTTTGTGCCTCCGCGGTACCTGCGGCCT
ACCGGGGGGAGAAACAGCATCCGTTACTCTGAGTTGGCACCCCTATTCGACACCACCCGTGTGTAC
CTGGTGGACAACAAGTCAACGGATGTGGCATCCCTGAACTACCAGAACGACCACAGCAACTTTCT
GACCACGGTCATTCAAAACAATGACTACAGCCCGGGGGAGGCAAGCACACAGACCATCAATCTTG
ACGACCGGTCGCACTGGGGCGGCGACCTGAAAACCATCCTGCATACCAACATGCCAAATGTGAAC
GAGTTCATGTTTACCAATAAGTTTAAGGCGCGGGTGATGGTGTCGCGCTTGCCTACTAAGGACAAT
CAGGTGGAGCTGAAATACGAGTGGGTGGAGTTCACGCTGCCCGAGGGCAACTACTCCGAGACCAT
GACCATAGACCTTATGAACAACGCGATCGTGGAGCACTACTTGAAAGTGGGCAGACAGAACGGGG
TTCTGGAAAGCGACATCGGGGTAAAGTTTGACACCCGCAACTTCAGACTGGGGTTTGACCCCGTCA
CTGGTCTTGTCATGCCTGGGGTATATACAAACGAAGCCTTCCATCCAGACATCATTTTGCTGCCAG
GATGCGGGGTGGACTTCACCCACAGCCGCCTGAGCAACTTGTTGGGCATCCGCAAGCGGCAACCC
TTCCAGGAGGGCTTTAGGATCACCTACGATGATCTGGAGGGTGGTAACATTCCCGCACTGTTGGAT
GTGGACGCCTACCAGGCGAGCTTGAAAGATGACACCGAACAGGGCGGGGGTGGCGCAGGCGGCA
GCAACAGCAGTGGCAGCGGCGCGGAAGAGAACTCCAACGCGGCAGCCGCGGCAATGCAGCCGGT
GGAGGACATGAACGATCATGCCATTCGCGGCGACACCTTTGCCACACGGGCTGAGGAGAAGCGCG
CTGAGGCCGAAGCAGCGGCCGAAGCTGCCGCCCCCGCTGCGCAACCCGAGGTCGAGAAGCCTCAG
AAGAAACCGGTGATCAAACCCCTGACAGAGGACAGCAAGAAACGCAGTTACAACCTAATAAGCA
ATGACAGCACCTTCACCCAGTACCGCAGCTGGTACCTTGCATACAACTACGGCGACCCTCAGACCG
GAATCCGCTCATGGACCCTGCTTTGCACTCCTGACGTAACCTGCGGCTCGGAGCAGGTCTACTGGT
CGTTGCCAGACATGATGCAAGACCCCGTGACCTTCCGCTCCACGCGCCAGATCAGCAACTTTCCGG
TGGTGGGCGCCGAGCTGTTGCCCGTGCACTCCAAGAGCTTCTACAACGACCAGGCCGTCTACTCCC
AACTCATCCGCCAGTTTACCTCTCTGACCCACGTGTTCAATCGCTTTCCCGAGAACCAGATTTTGGC
GCGCCCGCCAGCCCCCACCATCACCACCGTCAGTGAAAACGTTCCTGCTCTCACAGATCACGGGAC
GCTACCGCTGCGCAACAGCATCGGAGGAGTCCAGCGAGTGACCATTACTGACGCCAGACGCCGCA
CCTGCCCCTACGTTTACAAGGCCCTGGGCATAGTCTCGCCGCGCGTCCTATCGAGCCGCACTTTTTG
AGCAAGCATGTCCATCCTTATATCGCCCAGCAATAACACAGGCTGGGGCCTGCGCTTCCCAAGCAA
GATGTTTGGCGGGGCCAAGAAGCGCTCCGACCAACACCCAGTGCGCGTGCGCGGGCACTACCGCG
CGCCCTGGGGCGCGCACAAACGCGGCCGCACTGGGCGCACCACCGTCGATGACGCCATCGACGCG
GTGGTGGAGGAGGCGCGCAACTACACGCCCACGCCGCCACCAGTGTCCACAGTGGACGCGGCCAT
TCAGACCGTGGTGCGCGGAGCCCGGCGCTATGCTAAAATGAAGAGACGGCGGAGGCGCGTAGCAC
GTCGCCACCGCCGCCGACCCGGCACTGCCGCCCAACGCGCGGCGGCGGCCCTGCTTAACCGCGCA
CGTCGCACCGGCCGACGGGCGGCCATGCGGGCCGCTCGAAGGCTGGCCGCGGGTATTGTCACTGT
GCCCCCCAGGTCCAGGCGACGAGCGGCCGCCGCAGCAGCCGCGGCCATTAGTGCTATGACTCAGG
GTCGCAGGGGCAACGTGTATTGGGTGCGCGACTCGGTTAGCGGCCTGCGCGTGCCCGTGCGCACC
CGCCCCCCGCGCAACTAGATTGCAAGAAAAAACTACTTAGACTCGTACTGTTGTATGTATCCAGCG
GCGGCGGCGCGCAACGAAGCTATGTCCAAGCGCAAAATCAAAGAAGAGATGCTCCAGGTCATCGC
GCCGGAGATCTATGGCCCCCCGAAGAAGGAAGAGCAGGATTACAAGCCCCGAAAGCTAAAGCGG
GTCAAAAAGAAAAAGAAAGATGATGATGATGAACTTGACGACGAGGTGGAACTGCTGCACGCTA
CCGCGCCCAGGCGACGGGTACAGTGGAAAGGTCGACGCGTAAAACGTGTTTTGCGACCCGGCACC
ACCGTAGTCTTTACGCCCGGTGAGCGCTCCACCCGCACCTACAAGCGCGTGTATGATGAGGTGTAC
GGCGACGAGGACCTGCTTGAGCAGGCCAACGAGCGCCTCGGGGAGTTTGCCTACGGAAAGCGGCA
TAAGGACATGCTGGCGTTGCCGCTGGACGAGGGCAACCCAACACCTAGCCTAAAGCCCGTAACAC
TGCAGCAGGTGCTGCCCGCGCTTGCACCGTCCGAAGAAAAGCGCGGCCTAAAGCGCGAGTCTGGT
GACTTGGCACCCACCGTGCAGCTGATGGTACCCAAGCGCCAGCGACTGGAAGATGTCTTGGAAAA
AATGACCGTGGAACCTGGGCTGGAGCCCGAGGTCCGCGTGCGGCCAATCAAGCAGGTGGCGCCGG
GACTGGGCGTGCAGACCGTGGACGTTCAGATACCCACTACCAGTAGCACCAGTATTGCCACCGCC
ACAGAGGGCATGGAGACACAAACGTCCCCGGTTGCCTCAGCGGTGGCGGATGCCGCGGTGCAGGC
GGTCGCTGCGGCCGCGTCCAAGACCTCTACGGAGGTGCAAACGGACCCGTGGATGTTTCGCGTTTC
AGCCCCCCGGCGCCCGCGCCGTTCGAGGAAGTACGGCGCCGCCAGCGCGCTACTGCCCGAATATG
CCCTACATCCTTCCATTGCGCCTACCCCCGGCTATCGTGGCTACACCTACCGCCCCAGAAGACGAG
CAACTACCCGACGCCGAACCACCACTGGAACCCGCCGCCGCCGTCGCCGTCGCCAGCCCGTGCTG
GCCCCGATTTCCGTGCGCAGGGTGGCTCGCGAAGGAGGCAGGACCCTGGTGCTGCCAACAGCGCG
CTACCACCCCAGCATCGTTTAAAAGCCGGTCTTTGTGGTTCTTGCAGATATGGCCCTCACCTGCCGC
CTCCGTTTCCCGGTGCCGGGATTCCGAGGAAGAATGCACCGTAGGAGGGGCATGGCCGGCCACGG
CCTGACGGGCGGCATGCGTCGTGCGCACCACCGGCGGCGGCGCGCGTCGCACCGTCGCATGCGCG
GCGGTATCCTGCCCCTCCTTATTCCACTGATCGCCGCGGCGATTGGCGCCGTGCCCGGAATTGCAT
CCGTGGCCTTGCAGGCGCAGAGACACTGATTAAAAACAAGTTGCATGTGGAAAAATCAAAATAAA
AAGTCTGGACTCTCACGCTCGCTTGGTCCTGTAACTATTTTGTAGAATGGAAGACATCAACTTTGC
GTCTCTGGCCCCGCGACACGGCTCGCGCCCGTTCATGGGAAACTGGCAAGATATCGGCACCAGCA
ATATGAGCGGTGGCGCCTTCAGCTGGGGCTCGCTGTGGAGCGGCATTAAAAATTTCGGTTCCACCG
TTAAGAACTATGGCAGCAAGGCCTGGAACAGCAGCACAGGCCAGATGCTGAGGGATAAGTTGAA
AGAGCAAAATTTCCAACAAAAGGTGGTAGATGGCCTGGCCTCTGGCATTAGCGGGGTGGTGGACC
TGGCCAACCAGGCAGTGCAAAATAAGATTAACAGTAAGCTTGATCCCCGCCCTCCCGTAGAGGAG
CCTCCACCGGCCGTGGAGACAGTGTCTCCAGAGGGGCGTGGCGAAAAGCGTCCGCGCCCCGACAG
GGAAGAAACTCTGGTGACGCAAATAGACGAGCCTCCCTCGTACGAGGAGGCACTAAAGCAAGGCC
TGCCCACCACCCGTCCCATCGCGCCCATGGCTACCGGAGTGCTGGGCCAGCACACACCCGTAACGC
TGGACCTGCCTCCCCCCGCCGACACCCAGCAGAAACCTGTGCTGCCAGGCCCGACCGCCGTTGTTG
TAACCCGTCCTAGCCGCGCGTCCCTGCGCCGCGCCGCCAGCGGTCCGCGATCGTTGCGGCCCGTAG
CCAGTGGCAACTGGCAAAGCACACTGAACAGCATCGTGGGTCTGGGGGTGCAATCCCTGAAGCGC
CGACGATGCTTCTGATAGCTAACGTGTCGTATGTGTGTCATGTATGCGTCCATGTCGCCGCCAGAG
GAGCTGCTGAGCCGCCGCGCGCCCGCTTTCCAAGATGGCTACCCCTTCGATGATGCCGCAGTGGTC
TTACATGCACATCTCGGGCCAGGACGCCTCGGAGTACCTGAGCCCCGGGCTGGTGCAGTTTGCCCG
CGCCACCGAGACGTACTTCAGCCTGAATAACAAGTTTAGAAACCCCACGGTGGCGCCTACGCACG
ACGTGACCACAGACCGGTCCCAGCGTTTGACGCTGCGGTTCATCCCTGTGGACCGTGAGGATACTG
CGTACTCGTACAAGGCGCGGTTCACCCTAGCTGTGGGTGATAACCGTGTGCTGGACATGGCTTCCA
CGTACTTTGACATCCGCGGCGTGCTGGACAGGGGCCCTACTTTTAAGCCCTACTCTGGCACTGCCT
ACAACGCCCTGGCTCCCAAGGGTGCCCCAAATCCTTGCGAATGGGATGAAGCTGCTACTGCTCTTG
AAATAAACCTAGAAGAAGAGGACGATGACAACGAAGACGAAGTAGACGAGCAAGCTGAGCAGCA
AAAAACTCACGTATTTGGGCAGGCGCCTTATTCTGGTATAAATATTACAAAGGAGGGTATTCAAAT
AGGTGTCGAAGGTCAAACACCTAAATATGCCGATAAAACATTTCAACCTGAACCTCAAATAGGAG
AATCTCAGTGGTACGAAACAGAAATTAATCATGCAGCTGGGAGAGTCCTAAAAAAGACTACCCCA
ATGAAACCATGTTACGGTTCATATGCAAAACCCACAAATGAAAATGGAGGGCAAGGCATTCTTGT
AAAGCAACAAAATGGAAAGCTAGAAAGTCAAGTGGAAATGCAATTTTTCTCAACTACTGAGGCAG
CCGCAGGCAATGGTGATAACTTGACTCCTAAAGTGGTATTGTACAGTGAAGATGTAGATATAGAA
ACCCCAGACACTCATATTTCTTACATGCCCACTATTAAGGAAGGTAACTCACGAGAACTAATGGGC
CAACAATCTATGCCCAACAGGCCTAATTACATTGCTTTTAGGGACAATTTTATTGGTCTAATGTATT
ACAACAGCACGGGTAATATGGGTGTTCTGGCGGGCCAAGCATCGCAGTTGAATGCTGTTGTAGATT
TGCAAGACAGAAACACAGAGCTTTCATACCAGCTTTTGCTTGATTCCATTGGTGATAGAACCAGGT
ACTTTTCTATGTGGAATCAGGCTGTTGACAGCTATGATCCAGATGTTAGAATTATTGAAAATCATG
GAACTGAAGATGAACTTCCAAATTACTGCTTTCCACTGGGAGGTGTGATTAATACAGAGACTCTTA
CCAAGGTAAAACCTAAAACAGGTCAGGAAAATGGATGGGAAAAAGATGCTACAGAATTTTCAGAT
AAAAATGAAATAAGAGTTGGAAATAATTTTGCCATGGAAATCAATCTAAATGCCAACCTGTGGAG
AAATTTCCTGTACTCCAACATAGCGCTGTATTTGCCCGACAAGCTAAAGTACAGTCCTTCCAACGT
AAAAATTTCTGATAACCCAAACACCTACGACTACATGAACAAGCGAGTGGTGGCTCCCGGGCTAG
TGGACTGCTACATTAACCTTGGAGCACGCTGGTCCCTTGACTATATGGACAACGTCAACCCATTTA
ACCACCACCGCAATGCTGGCCTGCGCTACCGCTCAATGTTGCTGGGCAATGGTCGCTATGTGCCCT
TCCACATCCAGGTGCCTCAGAAGTTCTTTGCCATTAAAAACCTCCTTCTCCTGCCGGGCTCATACAC
CTACGAGTGGAACTTCAGGAAGGATGTTAACATGGTTCTGCAGAGCTCCCTAGGAAATGACCTAA
GGGTTGACGGAGCCAGCATTAAGTTTGATAGCATTTGCCTTTACGCCACCTTCTTCCCCATGGCCC
ACAACACCGCCTCCACGCTTGAGGCCATGCTTAGAAACGACACCAACGACCAGTCCTTTAACGACT
ATCTCTCCGCCGCCAACATGCTCTACCCTATACCCGCCAACGCTACCAACGTGCCCATATCCATCC
CCTCCCGCAACTGGGCGGCTTTCCGCGGCTGGGCCTTCACGCGCCTTAAGACTAAGGAAACCCCAT
CACTGGGCTCGGGCTACGACCCTTATTACACCTACTCTGGCTCTATACCCTACCTAGATGGAACCTT
TTACCTCAACCACACCTTTAAGAAGGTGGCCATTACCTTTGACTCTTCTGTCAGCTGGCCTGGCAAT
GACCGCCTGCTTACCCCCAACGAGTTTGAAATTAAGCGCTCAGTTGACGGGGAGGGTTACAACGTT
GCCCAGTGTAACATGACCAAAGACTGGTTCCTGGTACAAATGCTAGCTAACTATAACATTGGCTAC
CAGGGCTTCTATATCCCAGAGAGCTACAAGGACCGCATGTACTCCTTCTTTAGAAACTTCCAGCCC
ATGAGCCGTCAGGTGGTGGATGATACTAAATACAAGGACTACCAACAGGTGGGCATCCTACACCA
ACACAACAACTCTGGATTTGTTGGCTACCTTGCCCCCACCATGCGCGAAGGACAGGCCTACCCTGC
TAACTTCCCCTATCCGCTTATAGGCAAGACCGCAGTTGACAGCATTACCCAGAAAAAGTTTCTTTG
CGATCGCACCCTTTGGCGCATCCCATTCTCCAGTAACTTTATGTCCATGGGCGCACTCACAGACCT
GGGCCAAAACCTTCTCTACGCCAACTCCGCCCACGCGCTAGACATGACTTTTGAGGTGGATCCCAT
GGACGAGCCCACCCTTCTTTATGTTTTGTTTGAAGTCTTTGACGTGGTCCGTGTGCACCAGCCGCAC
CGCGGCGTCATCGAAACCGTGTACCTGCGCACGCCCTTCTCGGCCGGCAACGCCACAACATAAAG
AAGCAAGCAACATCAACAACAGCTGCCGCCATGGGCTCCAGTGAGCAGGAACTGAAAGCCATTGT
CAAAGATCTTGGTTGTGGGCCATATTTTTTGGGCACCTATGACAAGCGCTTTCCAGGCTTTGTTTCT
CCACACAAGCTCGCCTGCGCCATAGTCAATACGGCCGGTCGCGAGACTGGGGGCGTACACTGGAT
GGCCTTTGCCTGGAACCCGCACTCAAAAACATGCTACCTCTTTGAGCCCTTTGGCTTTTCTGACCAG
CGACTCAAGCAGGTTTACCAGTTTGAGTACGAGTCACTCCTGCGCCGTAGCGCCATTGCTTCTTCC
CCCGACCGCTGTATAACGCTGGAAAAGTCCACCCAAAGCGTACAGGGGCCCAACTCGGCCGCCTG
TGGACTATTCTGCTGCATGTTTCTCCACGCCTTTGCCAACTGGCCCCAAACTCCCATGGATCACAAC
CCCACCATGAACCTTATTACCGGGGTACCCAACTCCATGCTCAACAGTCCCCAGGTACAGCCCACC
CTGCGTCGCAACCAGGAACAGCTCTACAGCTTCCTGGAGCGCCACTCGCCCTACTTCCGCAGCCAC
AGTGCGCAGATTAGGAGCGCCACTTCTTTTTGTCACTTGAAAAACATGTAAAAATAATGTACTAGA
GACACTTTCAATAAAGGCAAATGCTTTTATTTGTACACTCTCGGGTGATTATTTACCCCCACCCTTG
CCGTCTGCGCCGTTTAAAAATCAAAGGGGTTCTGCCGCGCATCGCTATGCGCCACTGGCAGGGACA
CGTTGCGATACTGGTGTTTAGTGCTCCACTTAAACTCAGGCACAACCATCCGCGGCAGCTCGGTGA
AGTTTTCACTCCACAGGCTGCGCACCATCACCAACGCGTTTAGCAGGTCGGGCGCCGATATCTTGA
AGTCGCAGTTGGGGCCTCCGCCCTGCGCGCGCGAGTTGCGATACACAGGGTTGCAGCACTGGAAC
ACTATCAGCGCCGGGTGGTGCACGCTGGCCAGCACGCTCTTGTCGGAGATCAGATCCGCGTCCAG
GTCCTCCGCGTTGCTCAGGGCGAACGGAGTCAACTTTGGTAGCTGCCTTCCCAAAAAGGGCGCGTG
CCCAGGCTTTGAGTTGCACTCGCACCGTAGTGGCATCAAAAGGTGACCGTGCCCGGTCTGGGCGTT
AGGATACAGCGCCTGCATAAAAGCCTTGATCTGCTTAAAAGCCACCTGAGCCTTTGCGCCTTCAGA
GAAGAACATGCCGCAAGACTTGCCGGAAAACTGATTGGCCGGACAGGCCGCGTCGTGCACGCAGC
ACCTTGCGTCGGTGTTGGAGATCTGCACCACATTTCGGCCCCACCGGTTCTTCACGATCTTGGCCTT
GCTAGACTGCTCCTTCAGCGCGCGCTGCCCGTTTTCGCTCGTCACATCCATTTCAATCACGTGCTCC
TTATTTATCATAATGCTTCCGTGTAGACACTTAAGCTCGCCTTCGATCTCAGCGCAGCGGTGCAGCC
ACAACGCGCAGCCCGTGGGCTCGTGATGCTTGTAGGTCACCTCTGCAAACGACTGCAGGTACGCCT
GCAGGAATCGCCCCATCATCGTCACAAAGGTCTTGTTGCTGGTGAAGGTCAGCTGCAACCCGCGGT
GCTCCTCGTTCAGCCAGGTCTTGCATACGGCCGCCAGAGCTTCCACTTGGTCAGGCAGTAGTTTGA
AGTTCGCCTTTAGATCGTTATCCACGTGGTACTTGTCCATCAGCGCGCGCGCAGCCTCCATGCCCTT
CTCCCACGCAGACACGATCGGCACACTCAGCGGGTTCATCACCGTAATTTCACTTTCCGCTTCGCT
GGGCTCTTCCTCTTCCTCTTGCGTCCGCATACCACGCGCCACTGGGTCGTCTTCATTCAGCCGCCGC
ACTGTGCGCTTACCTCCTTTGCCATGCTTGATTAGCACCGGTGGGTTGCTGAAACCCACCATTTGTA
GCGCCACATCTTCTCTTTCTTCCTCGCTGTCCACGATTACTAATACGACTCACTATAGGTGTGGAAT
TTCACAGGAGGTACAGCTATGACCATGATTACGGATTCACTGGCCGTCGTTTTACAACGTCGTGAC
TGGGAAAACCCTGGCGTTACCCAACTTAATCGCCTTGCAGCACATCCCCCTTTCGCCAGCTGGCGT
AATAGCGAAGAGGCCCGCACCGATCGCCCTTCCCAACAGTTGCGCAGCCTGAATGGCGAATAGGT
CGCGCCGCACCGCGTCCGCGCTCGGGGGTGGTTTCGCGCTGCTCCTCTTCCCGACTGGCCATTTCCT
TCTCCTATAGGCAGAAAAAGATCATGGAGTCAGTCGAGAAGAAGGACAGCCTAACCGCCCCCTCT
GAGTTCGCCACCACCGCCTCCACCGATGCCGCCAACGCGCCTACCACCTTCCCCGTCGAGGCACCC
CCGCTTGAGGAGGAGGAAGTGATTATCGAGCAGGACCCAGGTTTTGTAAGCGAAGACGACGAGGA
CCGCTCAGTACCAACAGAGGATAAAAAGCAAGACCAGGACAACGCAGAGGCAAACGAGGAACAA
GTCGGGCGGGGGGACGAAAGGCATGGCGACTACCTAGATGTGGGAGACGACGTGCTGTTGAAGC
ATCTGCAGCGCCAGTGCGCCATTATCTGCGACGCGTTGCAAGAGCGCAGCGATGTGCCCCTCGCCA
TAGCGGATGTCAGCCTTGCCTACGAACGCCACCTATTCTCACCGCGCGTACCCCCCAAACGCCAAG
AAAACGGCACATGCGAGCCCAACCCGCGCCTCAACTTCTACCCCGTATTTGCCGTGCCAGAGGTGC
TTGCCACCTATCACATCTTTTTCCAAAACTGCAAGATACCCCTATCCTGCCGTGCCAACCGCAGCC
GAGCGGACAAGCAGCTGGCCTTGCGGCAGGGCGCTGTCATACCTGATATCGCCTCGCTCAACGAA
GTGCCAAAAATCTTTGAGGGTCTTGGACGCGACGAGAAGCGCGCGGCAAACGCTCTGCAACAGGA
AAACAGCGAAAATGAAAGTCACTCTGGAGTGTTGGTGGAACTCGAGGGTGACAACGCGCGCCTAG
CCGTACTAAAACGCAGCATCGAGGTCACCCACTTTGCCTACCCGGCACTTAACCTACCCCCCAAGG
TCATGAGCACAGTCATGAGTGAGCTGATCGTGCGCCGTGCGCAGCCCCTGGAGAGGGATGCAAAT
TTGCAAGAACAAACAGAGGAGGGCCTACCCGCAGTTGGCGACGAGCAGCTAGCGCGCTGGCTTCA
AACGCGCGAGCCTGCCGACTTGGAGGAGCGACGCAAACTAATGATGGCCGCAGTGCTCGTTACCG
TGGAGCTTGAGTGCATGCAGCGGTTCTTTGCTGACCCGGAGATGCAGCGCAAGCTAGAGGAAACA
TTGCACTACACCTTTCGACAGGGCTACGTACGCCAGGCCTGCAAGATCTCCAACGTGGAGCTCTGC
AACCTGGTCTCCTACCTTGGAATTTTGCACGAAAACCGCCTTGGGCAAAACGTGCTTCATTCCACG
CTCAAGGGCGAGGCGCGCCGCGACTACGTCCGCGACTGCGTTTACTTATTTCTATGCTACACCTGG
CAGACGGCCATGGGCGTTTGGCAGCAGTGCTTGGAGGAGTGCAACCTCAAGGAGCTGCAGAAACT
GCTAAAGCAAAACTTGAAGGACCTATGGACGGCCTTCAACGAGCGCTCCGTGGCCGCGCACCTGG
CGGACATCATTTTCCCCGAACGCCTGCTTAAAACCCTGCAACAGGGTCTGCCAGACTTCACCAGTC
AAAGCATGTTGCAGAACTTTAGGAACTTTATCCTAGAGCGCTCAGGAATCTTGCCCGCCACCTGCT
GTGCACTTCCTAGCGACTTTGTGCCCATTAAGTACCGCGAATGCCCTCCGCCGCTTTGGGGCCACT
GCTACCTTCTGCAGCTAGCCAACTACCTTGCCTACCACTCTGACATAATGGAAGACGTGAGCGGTG
ACGGTCTACTGGAGTGTCACTGTCGCTGCAACCTATGCACCCCGCACCGCTCCCTGGTTTGCAATT
CGCAGCTGCTTAACGAAAGTCAAATTATCGGTACCTTTGAGCTGCAGGGTCCCTCGCCTGACGAAA
AGTCCGCGGCTCCGGGGTTGAAACTCACTCCGGGGCTGTGGACGTCGGCTTACCTTCGCAAATTTG
TACCTGAGGACTACCACGCCCACGAGATTAGGTTCTACGAAGACCAATCCCGCCCGCCTAATGCG
GAGCTTACCGCCTGCGTCATTACCCAGGGCCACATTCTTGGCCAATTGCAAGCCATCAACAAAGCC
CGCCAAGAGTTTCTGCTACGAAAGGGACGGGGGGTTTACTTGGACCCCCAGTCCGGCGAGGAGCT
CAACCCAATCCCCCCGCCGCCGCAGCCCTATCAGCAGCAGCCGCGGGCCCTTGCTTCCCAGGATGG
CACCCAAAAAGAAGCTGCAGCTGCCGCCGCCACCCACGGACGAGGAGGAATACTGGGACAGTCA
GGCAGAGGAGGTTTTGGACGAGGAGGAGGAGGACATGATGGAAGACTGGGAGAGCCTAGACGAG
GAAGCTTCCGAGGTCGAAGAGGTGTCAGACGAAACACCGTCACCCTCGGTCGCATTCCCCTCGCC
GGCGCCCCAGAAATCGGCAACCGGTTCCAGCATGGCTACAACCTCCGCTCCTCAGGCGCCGCCGG
CACTGCCCGTTCGCCGACCCAACCGTAGATGGGACACCACTGGAACCAGGGCCGGTAAGTCCAAG
CAGCCGCCGCCGTTAGCCCAAGAGCAACAACAGCGCCAAGGCTACCGCTCATGGCGCGGGCACAA
GAACGCCATAGTTGCTTGCTTGCAAGACTGTGGGGGCAACATCTCCTTCGCCCGCCGCTTTCTTCTC
TACCATCACGGCGTGGCCTTCCCCCGTAACATCCTGCATTACTACCGTCATCTCTACAGCCCATACT
GCACCGGCGGCAGCGGCAGCAACAGCAGCGGCCACACAGAAGCAAAGGCGACCGGATAGCAAGA
CTCTGACAAAGCCCAAGAAATCCACAGCGGCGGCAGCAGCAGGAGGAGGAGCGCTGCGTCTGGC
GCCCAACGAACCCGTATCGACCCGCGAGCTTAGAAACAGGATTTTTCCCACTCTGTATGCTATATT
TCAACAGAGCAGGGGCCAAGAACAAGAGCTGAAAATAAAAAACAGGTCTCTGCGATCCCTCACCC
GCAGCTGCCTGTATCACAAAAGCGAAGATCAGCTTCGGCGCACGCTGGAAGACGCGGAGGCTCTC
TTCAGTAAATACTGCGCGCTGACTCTTAAGGACTAGTTTCGCGCCCTTTCTCAAATTTAAGCGCGA
AAACTACGTCATCTCCAGCGGCCACACCCGGCGCCAGCACCTGTTGTCAGCGCCATTATGAGCAAG
GAAATTCCCACGCCCTACATGTGGAGTTACCAGCCACAAATGGGACTTGCGGCTGGAGCTGCCCA
AGACTACTCAACCCGAATAAACTACATGAGCGCGGGACCCCACATGATATCCCGGGTCAACGGAA
TACGCGCCCACCGAAACCGAATTCTCCTGGAACAGGCGGCTATTACCACCACACCTCGTAATAACC
TTAATCCCCGTAGTTGGCCCGCTGCCCTGGTGTACCAGGAAAGTCCCGCTCCCACCACTGTGGTAC
TTCCCAGAGACGCCCAGGCCGAAGTTCAGATGACTAACTCAGGGGCGCAGCTTGCGGGCGGCTTT
CGTCACAGGGTGCGGTCGCCCGGGCAGGGTATAACTCACCTGACAATCAGAGGGCGAGGTATTCA
GCTCAACGACGAGTCGGTGAGCTCCTCGCTTGGTCTCCGTCCGGACGGGACATTTCAGATCGGCGG
CGCCGGCCGCTCTTCATTCACGCCTCGTCAGGCAATCCTAACTCTGCAGACCTCGTCCTCTGAGCC
GCGCTCTGGAGGCATTGGAACTCTGCAATTTATTGAGGAGTTTGTGCCATCGGTCTACTTTAACCC
CTTCTCGGGACCTCCCGGCCACTATCCGGATCAATTTATTCCTAACTTTGACGCGGTAAAGGACTC
GGCGGACGGCTACGACTGAATGTTAAGTGGAGAGGCAGAGCAACTGCGCCTGAAACACCTGGTCC
ACTGTCGCCGCCACAAGTGCTTTGCCCGCGACTCCGGTGAGTTTTGCTACTTTGAATTGCCCGAGG
ATCATATCGAGGGCCCGGCGCACGGCGTCCGGCTTACCGCCCAGGGAGAGCTTGCCCGTAGCCTG
ATTCGGGAGTTTACCCAGCGCCCCCTGCTAGTTGAGCGGGACAGGGGACCCTGTGTTCTCACTGTG
ATTTGCAACTGTCCTAACCCTGGATTACATCAAGATCTTTGTTGCCATCTCTGTGCTGAGTATAATA
AATACAGAAATTAAAATATACTGGGGCTCCTATCGCCATCCTGTAAACGCCACCGTCTTCACCCGC
CCAAGCAAACCAAGGCGAACCTTACCTGGTACTTTTAACATCTCTCCCTCTGTGATTTACAACAGT
TTCAACCCAGACGGAGTGAGTCTACGAGAGAACCTCTCCGAGCTCAGCTACTCCATCAGAAAAAA
CACCACCCTCCTTACCTGCCGGGAACGTACGAGTGCGTCACCGGCCGCTGCACCACACCTACCGCC
TGACCGTAAACCAGACTTTTTCCGGACAGACCTCAATAACTCTGTTTACCAGAACAGGAGGTGAGC
TTAGAAAACCCTTAGGGTATTAGGCCAAAGGCGCAGCTACTGTGGGGTTTATGAACAATTCAAGC
AACTCTACGGGCTATTCTAATTCAGGTTTCTCTAGAAATGGACGGAATTATTACAGAGCAGCGCCT
GCTAGAAAGACGCAGGGCAGCGGCCGAGCAACAGCGCATGAATCAAGAGCTCCAAGACATGGTT
AACTTGCACCAGTGCAAAAGGGGTATCTTTTGTCTGGTAAAGCAGGCCAAAGTCACCTACGACAG
TAATACCACCGGACACCGCCTTAGCTACAAGTTGCCAACCAAGCGTCAGAAATTGGTGGTCATGGT
GGGAGAAAAGCCCATTACCATAACTCAGCACTCGGTAGAAACCGAAGGCTGCATTCACTCACCTT
GTCAAGGACCTGAGGATCTCTGCACCCTTATTAAGACCCTGTGCGGTCTCAAAGATCTTATTCCCTT
TAACTAATAAAAAAAAATAATAAAGCATCACTTACTTAAAATCAGTTAGCAAATTTCTGTCCAGTT
TATTCAGCAGCACCTCCTTGCCCTCCTCCCAGCTCTGGTATTGCAGCTTCCTCCTGGCTGCAAACTT
TCTCCACAATCTAAATGGAATGTCAGTTTCCTCCTGTTCCTGTCCATCCGCACCCACTATCTTCATG
TTGTTGCAGATGAAGCGCGCAAGACCGTCTGAAGATACCTTCAACCCCGTGTATCCATATGACACG
GAAACCGGTCCTCCAACTGTGCCTTTTCTTACTCCTCCCTTTGTATCCCCCAATGGGTTTCAAGAGA
GTCCCCCTGGGGTACTCTCTTTGCGCCTATCCGAACCTCTAGTTACCTCCAATGGCATGCTTGCGCT
CAAAATGGGCAACGGCCTCTCTCTGGACGAGGCCGGCAACCTTACCTCCCAAAATGTAACCACTGT
GAGCCCACCTCTCAAAAAAACCAAGTCAAACATAAACCTGGAAATATCTGCACCCCTCACAGTTA
CCTCAGAAGCCCTAACTGTGGCTGCCGCCGCACCTCTAATGGTCGCGGGCAACACACTCACCATGC
AATCACAGGCCCCGCTAACCGTGCACGACTCCAAACTTAGCATTGCCACCCAAGGACCCCTCACA
GTGTCAGAAGGAAAGCTAGCCCTGCAAACATCAGGCCCCCTCACCACCACCGATAGCAGTACCCT
TACTATCACTGCCTCACCCCCTCTAACTACTGCCACTGGTAGCTTGGGCATTGACTTGAAAGAGCC
CATTTATACACAAAATGGAAAACTAGGACTAAAGTACGGGGCTCCTTTGCATGTAACAGACGACC
TAAACACTTTGACCGTAGCAACTGGTCCAGGTGTGACTATTAATAATACTTCCTTGCAAACTAAAG
TTACTGGAGCCTTGGGTTTTGATTCACAAGGCAATATGCAACTTAATGTAGCAGGAGGACTAAGGA
TTGATTCTCAAAACAGACGCCTTATACTTGATGTTAGTTATCCGTTTGATGCTCAAAACCAACTAA
ATCTAAGACTAGGACAGGGCCCTCTTTTTATAAACTCAGCCCACAACTTGGATATTAACTACAACA
AAGGCCTTTACTTGTTTACAGCTTCAAACAATTCCAAAAAGCTTGAGGTTAACCTAAGCACTGCCA
AGGGGTTGATGTTTGACGCTACAGCCATAGCCATTAATGCAGGAGATGGGCTTGAATTTGGTTCAC
CTAATGCACCAAACACAAATCCCCTCAAAACAAAAATTGGCCATGGCCTAGAATTTGATTCAAAC
AAGGCTATGGTTCCTAAACTAGGAACTGGCCTTAGTTTTGACAGCACAGGTGCCATTACAGTAGGA
AACAAAAATAATGATAAGCTAACTTTGTGGACCACACCAGCTCCATCTCCTAACTGTAGACTAAAT
GCAGAGAAAGATGCTAAACTCACTTTGGTCTTAACAAAATGTGGCAGTCAAATACTTGCTACAGTT
TCAGTTTTGGCTGTTAAAGGCAGTTTGGCTCCAATATCTGGAACAGTTCAAAGTGCTCATCTTATTA
TAAGATTTGACGAAAATGGAGTGCTACTAAACAATTCCTTCCTGGACCCAGAATATTGGAACTTTA
GAAATGGAGATCTTACTGAAGGCACAGCCTATACAAACGCTGTTGGATTTATGCCTAACCTATCAG
CTTATCCAAAATCTCACGGTAAAACTGCCAAAAGTAACATTGTCAGTCAAGTTTACTTAAACGGAG
ACAAAACTAAACCTGTAACACTAACCATTACACTAAACGGTACACAGGAAACAGGAGACACAACT
CCAAGTGCATACTCTATGTCATTTTCATGGGACTGGTCTGGCCACAACTACATTAATGAAATATTT
GCCACATCCTCTTACACTTTTTCATACATTGCCCAAGAATAAAGAATCGTTTGTGTTATGTTTCAAC
GTGTTTATTTTTCAATTGCAGAAAATTTCGAATCATTTTTCATTCAGTAGTATAGCCCCACCACCAC
ATAGCTTATACAGATCACCGTACCTTAATCAAACTCACAGAACCCTAGTATTCAACCTGCCACCTC
CCTCCCAACACACAGAGTACACAGTCCTTTCTCCCCGGCTGGCCTTAAAAAGCATCATATCATGGG
TAACAGACATATTCTTAGGTGTTATATTCCACACGGTTTCCTGTCGAGCCAAACGCTCATCAGTGA
TATTAATAAACTCCCCGGGCAGCTCACTTAAGTTCATGTCGCTGTCCAGCTGCTGAGCCACAGGCT
GCTGTCCAACTTGCGGTTGCTTAACGGGCGGCGAAGGAGAAGTCCACGCCTACATGGGGGTAGAG
TCATAATCGTGCATCAGGATAGGGCGGTGGTGCTGCAGCAGCGCGCGAATAAACTGCTGCCGCCG
CCGCTCCGTCCTGCAGGAATACAACATGGCAGTGGTCTCCTCAGCGATGATTCGCACCGCCCGCAG
CATAAGGCGCCTTGTCCTCCGGGCACAGCAGCGCACCCTGATCTCACTTAAATCAGCACAGTAACT
GCAGCACAGCACCACAATATTGTTCAAAATCCCACAGTGCAAGGCGCTGTATCCAAAGCTCATGG
CGGGGACCACAGAACCCACGTGGCCATCATACCACAAGCGCAGGTAGATTAAGTGGCGACCCCTC
ATAAACACGCTGGACATAAACATTACCTCTTTTGGCATGTTGTAATTCACCACCTCCCGGTACCAT
ATAAACCTCTGATTAAACATGGCGCCATCCACCACCATCCTAAACCAGCTGGCCAAAACCTGCCCG
CCGGCTATACACTGCAGGGAACCGGGACTGGAACAATGACAGTGGAGAGCCCAGGACTCGTAACC
ATGGATCATCATGCTCGTCATGATATCAATGTTGGCACAACACAGGCACACGTGCATACACTTCCT
CAGGATTACAAGCTCCTCCCGCGTTAGAACCATATCCCAGGGAACAACCCATTCCTGAATCAGCGT
AAATCCCACACTGCAGGGAAGACCTCGCACGTAACTCACGTTGTGCATTGTCAAAGTGTTACATTC
GGGCAGCAGCGGATGATCCTCCAGTATGGTAGCGCGGGTTTCTGTCTCAAAAGGAGGTAGACGAT
CCCTACTGTACGGAGTGCGCCGAGACAACCGAGATCGTGTTGGTCGTAGTGTCATGCCAAATGGA
ACGCCGGACGTAGTCATATTTCCTGAAGCAAAACCAGGTGCGGGCGTGACAAACAGATCTGCGTC
TCCGGTCTCGCCGCTTAGATCGCTCTGTGTAGTAGTTGTAGTATATCCACTCTCTCAAAGCATCCAG
GCGCCCCCTGGCTTCGGGTTCTATGTAAACTCCTTCATGCGCCGCTGCCCTGATAACATCCACCACC
GCAGAATAAGCCACACCCAGCCAACCTACACATTCGTTCTGCGAGTCACACACGGGAGGAGCGGG
AAGAGCTGGAAGAACCATGTTTTTTTTTTTATTCCAAAAGATTATCCAAAACCTCAAAATGAAGAT
CTATTAAGTGAACGCGCTCCCCTCCGGTGGCGTGGTCAAACTCTACAGCCAAAGAACAGATAATG
GCATTTGTAAGATGTTGCACAATGGCTTCCAAAAGGCAAACGGCCCTCACGTCCAAGTGGACGTA
AAGGCTAAACCCTTCAGGGTGAATCTCCTCTATAAACATTCCAGCACCTTCAACCATGCCCAAATA
ATTCTCATCTCGCCACCTTCTCAATATATCTCTAAGCAAATCCCGAATATTAAGTCCGGCCATTGTA
AAAATCTGCTCCAGAGCGCCCTCCACCTTCAGCCTCAAGCAGCGAATCATGATTGCAAAAATTCAG
GTTCCTCACAGACCTGTATAAGATTCAAAAGCGGAACATTAACAAAAATACCGCGATCCCGTAGG
TCCCTTCGCAGGGCCAGCTGAACATAATCGTGCAGGTCTGCACGGACCAGCGCGGCCACTTCCCCG
CCAGGAACCATGACAAAAGAACCCACACTGATTATGACACGCATACTCGGAGCTATGCTAACCAG
CGTAGCCCCGATGTAAGCTTGTTGCATGGGCGGCGATATAAAATGCAAGGTGCTGCTCAAAAAAT
CAGGCAAAGCCTCGCGCAAAAAAGAAAGCACATCGTAGTCATGCTCATGCAGATAAAGGCAGGTA
AGCTCCGGAACCACCACAGAAAAAGACACCATTTTTCTCTCAAACATGTCTGCGGGTTTCTGCATA
AACACAAAATAAAATAACAAAAAAACATTTAAACATTAGAAGCCTGTCTTACAACAGGAAAAACA
ACCCTTATAAGCATAAGACGGACTACGGCCATGCCGGCGTGACCGTAAAAAAACTGGTCACCGTG
ATTAAAAAGCACCACCGACAGCTCCTCGGTCATGTCCGGAGTCATAATGTAAGACTCGGTAAACA
CATCAGGTTGATTCACATCGGTCAGTGCTAAAAAGCGACCGAAATAGCCCGGGGGAATACATACC
CGCAGGCGTAGAGACAACATTACAGCCCCCATAGGAGGTATAACAAAATTAATAGGAGAGAAAA
ACACATAAACACCTGAAAAACCCTCCTGCCTAGGCAAAATAGCACCCTCCCGCTCCAGAACAACA
TACAGCGCTTCCACAGCGGCAGCCATAACAGTCAGCCTTACCAGTAAAAAAGAAAACCTATTAAA
AAAACACCACTCGACACGGCACCAGCTCAATCAGTCACAGTGTAAAAAAGGGCCAAGTGCAGAGC
GAGTATATATAGGACTAAAAAATGACGTAACGGTTAAAGTCCACAAAAAACACCCAGAAAACCGC
ACGCGAACCTACGCCCAGAAACGAAAGCCAAAAAACCCACAACTTCCTCAAATCGTCACTTCCGT
TTTCCCACGTTACGTCACTTCCCATTTTAAGAAAACTACAATTCCCAACACATACAAGTTACTCCGC
CCTAAAACCTACGTCACCCGCCCCGTTCCCACGCCCCGCGCCACGTCACAAACTCCACCCCCTCAT
TATCATATTGGCTTCAATCCAAAATAAGGTATATTATTGATGATGTTAATTAATTTAAATCCGCATG
CGATATCGAGCTCTCCCGGGAATTCGGATCTGCGACGCGAGGCTGGATGGCCTTCCCCATTATGAT
TCTTCTCGCGTTTAAGGGCACCAATAACTGCCTTAAAAAAATTACGCCCCGCCCTGCCACTCATCG
CAGTACTGTTGTAATTCATTAAGCATTCTGCCGACATGGAAGCCATCACAAACGGCATGATGAACC
TGAATCGCCAGCGGCATCAGCACCTTGTCGCCTTGCGTATAATATTTGCCCATGGTGAAAACGGGG
GCGAAGAAGTTGTCCATATTGGCCACGTTTAAATCAAAACTGGTGAAACTCACCCAGGGATTGGCT
GAGACGAAAAACATATTCTCAATAAACCCTTTAGGGAAATAGGCCAGGTTTTCACCGTAACACGC
CACATCTTGCGAATATATGTGTAGAAACTGCCGGAAATCGTCGTGGTATTCACTCCAGAGCGATGA
AAACGTTTCAGTTTGCTCATGGAAAACGGTGTAACAAGGGTGAACACTATCCCATATCACCAGCTC
ACCGTCTTTCATTGCCATACGGAATTCCGGATGAGCATTCATCAGGCGGGCAAGAATGTGAATAAA
GGCCGGATAAAACTTGTGCTTATTTTTCTTTACGGTCTTTAAAAAGGCCGTAATATCCAGCTGAAC
GGTCTGGTTATAGGTACATTGAGCAACTGACTGAAATGCCTCAAAATGTTCTTTACGATGCCATTG
GGATATATCAACGGTGGTATATCCAGTGATTTTTTTCTCCATTTTAGCTTCCTTAGCTCCTGAAAAT
CTCGATAACTCAAAAAATACGCCCGGTAGTGATCTTATTTCATTATGGTGAAAGTTGGAACCTCTT
ACGTGCCGATCAACGTCTCATTTTCGCCAAAAGTTGGCCCAGGGCTTCCCGGTATCAACAGGGACA
CCAGGATTTATTTATTCTGCGAAGTGATCTTCCGTCACAGGTATTTATTCGCGATAAGCTCATGGAG
CGGCGTAACCGTCGCACAGGAAGGACAGAGAAAGCGCGGATCTGGGAAGTGACGGACAGAACGG
TCAGGACCTGGATTGGGGAGGCGGTTGCCGCCGCTGCTGCTGACGGTGTGACGTTCTCTGTTCCGG
TCACACCACATACGTTCCGCCATTCCTATGCGATGCACATGCTGTATGCCGGTATACCGCTGAAAG
TTCTGCAAAGCCTGATGGGACATAAGTCCATCAGTTCAACGGAAGTCTACACGAAGGTTTTTGCGC
TGGATGTGGCTGCCCGGCACCGGGTGCAGTTTGCGATGCCGGAGTCTGATGCGGTTGCGATGCTGA
AACAATTATCCTGAGAATAAATGCCTTGGCCTTTATATGGAAATGTGGAACTGAGTGGATATGCTG
TTTTTGTCTGTTAAACAGAGAAGCTGGCTGTTATCCACTGAGAAGCGAACGAAACAGTCGGGAAA
ATCTCCCATTATCGTAGAGATCCGCATTATTAATCTCAGGAGCCTGTGTAGCGTTTATAGGAAGTA
GTGTTCTGTCATGATGCCTGCAAGCGGTAACGAAAACGATTTGAATATGCCTTCAGGAACAATAGA
AATCTTCGTGCGGTGTTACGTTGAAGTGGAGCGGATTATGTCAGCAATGGACAGAACAACCTAAT
GAACACAGAACCATGATGTGGTCTGTCCTTTTACAGCCAGTAGTGCTCGCCGCAGTCGAGCGACAG
GGCGAAGCCCTCGAGTGAGCGAGGAAGCACCAGGGAACAGCACTTATATATTCTGCTTACACACG
ATGCCTGAAAAAACTTCCCTTGGGGTTATCCACTTATCCACGGGGATATTTTTATAATTATTTTTTT
TATAGTTTTTAGATCTTCTTTTTTAGAGCGCCTTGTAGGCCTTTATCCATGCTGGTTCTAGAGAAGG
TGTTGTGACAAATTGCCCTTTCAGTGTGACAAATCACCCTCAAATGACAGTCCTGTCTGTGACAAA
TTGCCCTTAACCCTGTGACAAATTGCCCTCAGAAGAAGCTGTTTTTTCACAAAGTTATCCCTGCTTA
TTGACTCTTTTTTATTTAGTGTGACAATCTAAAAACTTGTCACACTTCACATGGATCTGTCATGGCG
GAAACAGCGGTTATCAATCACAAGAAACGTAAAAATAGCCCGCGAATCGTCCAGTCAAACGACCT
CACTGAGGCGGCATATAGTCTCTCCCGGGATCAAAAACGTATGCTGTATCTGTTCGTTGACCAGAT
CAGAAAATCTGATGGCACCCTACAGGAACATGACGGTATCTGCGAGATCCATGTTGCTAAATATG
CTGAAATATTCGGATTGACCTCTGCGGAAGCCAGTAAGGATATACGGCAGGCATTGAAGAGTTTC
GCGGGGAAGGAAGTGGTTTTTTATCGCCCTGAAGAGGATGCCGGCGATGAAAAAGGCTATGAATC
TTTTCCTTGGTTTATCAAACGTGCGCACAGTCCATCCAGAGGGCTTTACAGTGTACATATCAACCC
ATATCTCATTCCCTTCTTTATCGGGTTACAGAACCGGTTTACGCAGTTTCGGCTTAGTGAAACAAAA
GAAATCACCAATCCGTATGCCATGCGTTTATACGAATCCCTGTGTCAGTATCGTAAGCCGGATGGC
TCAGGCATCGTCTCTCTGAAAATCGACTGGATCATAGAGCGTTACCAGCTGCCTCAAAGTTACCAG
CGTATGCCTGACTTCCGCCGCCGCTTCCTGCAGGTCTGTGTTAATGAGATCAACAGCAGAACTCCA
ATGCGCCTCTCATACATTGAGAAAAAGAAAGGCCGCCAGACGACTCATATCGTATTTTCCTTCCGC
GATATCACTTCCATGACGACAGGATAGTCTGAGGGTTATCTGTCACAGATTTGAGGGTGGTTCGTC
ACATTTGTTCTGACCTACTGAGGGTAATTTGTCACAGTTTTGCTGTTTCCTTCAGCCTGCATGGATT
TTCTCATACTTTTTGAACTGTAATTTTTAAGGAAGCCAAATTTGAGGGCAGTTTGTCACAGTTGATT
TCCTTCTCTTTCCCTTCGTCATGTGACCTGATATCGGGGGTTAGTTCGTCATCATTGATGAGGGTTG
ATTATCACAGTTTATTACTCTGAATTGGCTATCCGCGTGTGTACCTCTACCTGGAGTTTTTCCCACG
GTGGATATTTCTTCTTGCGCTGAGCGTAAGAGCTATCTGACAGAACAGTTCTTCTTTGCTTCCTCGC
CAGTTCGCTCGCTATGCTCGGTTACACGGCTGCGGCGAGCGCTAGTGATAATAAGTGACTGAGGTA
TGTGCTCTTCTTATCTCCTTTTGTAGTGTTGCTCTTATTTTAAACAACTTTGCGGTTTTTTGATGACT
TTGCGATTTTGTTGTTGCTTTGCAGTAAATTGCAAGATTTAATAAAAAAACGCAAAGCAATGATTA
AAGGATGTTCAGAATGAAACTCATGGAAACACTTAACCAGTGCATAAACGCTGGTCATGAAATGA
CGAAGGCTATCGCCATTGCACAGTTTAATGATGACAGCCCGGAAGCGAGGAAAATAACCCGGCGC
TGGAGAATAGGTGAAGCAGCGGATTTAGTTGGGGTTTCTTCTCAGGCTATCAGAGATGCCGAGAA
AGCAGGGCGACTACCGCACCCGGATATGGAAATTCGAGGACGGGTTGAGCAACGTGTTGGTTATA
CAATTGAACAAATTAATCATATGCGTGATGTGTTTGGTACGCGATTGCGACGTGCTGAAGACGTAT
TTCCACCGGTGATCGGGGTTGCTGCCCATAAAGGTGGCGTTTACAAAACCTCAGTTTCTGTTCATCT
TGCTCAGGATCTGGCTCTGAAGGGGCTACGTGTTTTGCTCGTGGAAGGTAACGACCCCCAGGGAAC
AGCCTCAATGTATCACGGATGGGTACCAGATCTTCATATTCATGCAGAAGACACTCTCCTGCCTTT
CTATCTTGGGGAAAAGGACGATGTCACTTATGCAATAAAGCCCACTTGCTGGCCGGGGCTTGACAT
TATTCCTTCCTGTCTGGCTCTGCACCGTATTGAAACTGAGTTAATGGGCAAATTTGATGAAGGTAA
ACTGCCCACCGATCCACACCTGATGCTCCGACTGGCCATTGAAACTGTTGCTCATGACTATGATGT
CATAGTTATTGACAGCGCGCCTAACCTGGGTATCGGCACGATTAATGTCGTATGTGCTGCTGATGT
GCTGATTGTTCCCACGCCTGCTGAGTTGTTTGACTACACCTCCGCACTGCAGTTTTTCGATATGCTT
CGTGATCTGCTCAAGAACGTTGATCTTAAAGGGTTCGAGCCTGATGTACGTATTTTGCTTACCAAA
TACAGCAATAGTAATGGCTCTCAGTCCCCGTGGATGGAGGAGCAAATTCGGGATGCCTGGGGAAG
CATGGTTCTAAAAAATGTTGTACGTGAAACGGATGAAGTTGGTAAAGGTCAGATCCGGATGAGAA
CTGTTTTTGAACAGGCCATTGATCAACGCTCTTCAACTGGTGCCTGGAGAAATGCTCTTTCTATTTG
GGAACCTGTCTGCAATGAAATTTTCGATCGTCTGATTAAACCACGCTGGGAGATTAGATAATGAAG
CGTGCGCCTGTTATTCCAAAACATACGCTCAATACTCAACCGGTTGAAGATACTTCGTTATCGACA
CCAGCTGCCCCGATGGTGGATTCGTTAATTGCGCGCGTAGGAGTAATGGCTCGCGGTAATGCCATT
ACTTTGCCTGTATGTGGTCGGGATGTGAAGTTTACTCTTGAAGTGCTCCGGGGTGATAGTGTTGAG
AAGACCTCTCGGGTATGGTCAGGTAATGAACGTGACCAGGAGCTGCTTACTGAGGACGCACTGGA
TGATCTCATCCCTTCTTTTCTACTGACTGGTCAACAGACACCGGCGTTCGGTCGAAGAGTATCTGGT
GTCATAGAAATTGCCGATGGGAGTCGCCGTCGTAAAGCTGCTGCACTTACCGAAAGTGATTATCGT
GTTCTGGTTGGCGAGCTGGATGATGAGCAGATGGCTGCATTATCCAGATTGGGTAACGATTATCGC
CCAACAAGTGCTTATGAACGTGGTCAGCGTTATGCAAGCCGATTGCAGAATGAATTTGCTGGAAAT
ATTTCTGCGCTGGCTGATGCGGAAAATATTTCACGTAAGATTATTACCCGCTGTATCAACACCGCC
AAATTGCCTAAATCAGTTGTTGCTCTTTTTTCTCACCCCGGTGAACTATCTGCCCGGTCAGGTGATG
CACTTCAAAAAGCCTTTACAGATAAAGAGGAATTACTTAAGCAGCAGGCATCTAACCTTCATGAG
CAGAAAAAAGCTGGGGTGATATTTGAAGCTGAAGAAGTTATCACTCTTTTAACTTCTGTGCTTAAA
ACGTCATCTGCATCAAGAACTAGTTTAAGCTCACGACATCAGTTTGCTCCTGGAGCGACAGTATTG
TATAAGGGCGATAAAATGGTGCTTAACCTGGACAGGTCTCGTGTTCCAACTGAGTGTATAGAGAA
AATTGAGGCCATTCTTAAGGAACTTGAAAAGCCAGCACCCTGATGCGACCACGTTTTAGTCTACGT
TTATCTGTCTTTACTTAATGTCCTTTGTTACAGGCCAGAAAGCATAACTGGCCTGAATATTCTCTCT
GGGCCCACTGTTCCACTTGTATCGTCGGTCTGATAATCAGACTGGGACCACGGTCCCACTCGTATC
GTCGGTCTGATTATTAGTCTGGGACCACGGTCCCACTCGTATCGTCGGTCTGATTATTAGTCTGGGA
CCACGGTCCCACTCGTATCGTCGGTCTGATAATCAGACTGGGACCACGGTCCCACTCGTATCGTCG
GTCTGATTATTAGTCTGGGACCATGGTCCCACTCGTATCGTCGGTCTGATTATTAGTCTGGGACCAC
GGTCCCACTCGTATCGTCGGTCTGATTATTAGTCTGGAACCACGGTCCCACTCGTATCGTCGGTCTG
ATTATTAGTCTGGGACCACGGTCCCACTCGTATCGTCGGTCTGATTATTAGTCTGGGACCACGATC
CCACTCGTGTTGTCGGTCTGATTATCGGTCTGGGACCACGGTCCCACTTGTATTGTCGATCAGACTA
TCAGCGTGAGACTACGATTCCATCAATGCCTGTCAAGGGCAAGTATTGACATGTCGTCGTAACCTG
TAGAACGGAGTAACCTCGGTGTGCGGTTGTATGCCTGCTGTGGATTGCTGCTGTGTCCTGCTTATCC
ACAACATTTTGCGCACGGTTATGTGGACAAAATACCTGGTTACCCAGGCCGTGCCGGCACGTTAAC
CGGGCACATTTCCCCGAAAAGTGCCACCTGACGTCTAAGAAACCATTATTATCATGACATTAACCT
ATAAAAATAGGCGTATCACGAGGCCCTTTCGTCTTCAAGAATTGGATCCGAATTCCCGGGAGAGCT
CGATATCGCATGCGGATTTAAATTAATTAA
* C1C
(SEQ ID NO: 84)
CATCATCAATAATATACCTTATTTTGGATTGAAGCCAATATGATAATGAGGGGGTGGAGTTTGTGA
CGTGGCGCGGGGCGTGGGAACGGGGCGGGTGACGTAGTAGTGTGGCGGAAGTGTGATGTTGCAAG
TGTGGCGGAACACATGTAAGCGACGGATGTGGCAAAAGTGACGTTTTTGGTGTGCGCCGGTGTAC
ACAGGAAGTGACAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAATTTTGTGTTACT
CATAGCGCGTAATATTTGTCTAGGGCCGCGGGGACTTTGACCGTTTACGTGGAGACTCGCCCAGGT
GTTTTTCTCAGGTGTTTTCCGCGTTCCGGGTCAAAGTTGGCGTTTTATTATTATAGTCAGTCGAAGC
TTGGATCCGGTACCTCTAGAATTCTCGAGCGGCCGCTAGCGACATCGGATCTCCCGATCCCCTATG
GTGCACTCTCAGTACAATCTGCTCTGATGCCGCATAGTTAAGCCAGTATCTGCTCCCTGCTTGTGTG
TTGGAGGTCGCTGAGTAGTGCGCGAGCAAAATTTAAGCTACAACAAGGCAAGGCTTGACCGACAA
TTGCATGAAGAATCTGCTTAGGGTTAGGCGTTTTGCGCTGCTTCGCGATGTACGGGCCAGATATAC
GCGTTGACATTGATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACG
TCAATGGGTGGACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCTCTG
GCTAACTAGAGAACCCACTGCTTACTGGCTTATCGAAATGAGACCCAAGCTGGCTAGTTAAGCTAT
CAACAAGTTTGTACAAAAAAGCAGGCTTTAAAGGAACCAATTCAGTCGACTGGATCCGGTACCAC
CATGTTCCTGAACTGCTGCCCAGGTTGCTGTATGGAGCCTGAATTCACCATGGTGAGCAAGGGCGA
GGAGCTGTTCACCGGGGTGGTGCCCATCCTGGTCGAGCTGGACGGCGACGTAAACGGCCACAAGT
TCAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTGGGGCGTGCAGTGC
TTCGCCCGCTACCCCGACCACATGAAGCAGCACGACTTCTTCAAGTCCGCCATGCCCGAAGGCTAC
GTCCAGGAGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGACGGCAAC
ATCCTGGGGCACAAGCTGGAGTACAACGCCATCAGCGACAACGTCTATATCACCGCCGACAAGCA
GAAGAACGGCATCAAGGCCAACTTCAAGATCCGCCACAACATCGAGGACGGCAGCGTGCAGCTCG
CCGACCACTACCAGCAGAACACCCCCATCGGCGACGGCCCCGTGCTGCTGCCCGACAACCACTAC
CTGAGCACCCAGTCCAAGCTGAGCAAAGACCCCAACGAGAAGCGCGATCACATGGTCCTGCTGGA
GTTCGTGACCGCCGCCGGGATCACTCTCGGCATGGACGAGCTGTACAAGGTCGACTATCCGTACGA
CGTACCAGACTACGCATAACCGCGGCCGCACTCGAGATATCTAGACCCAGCTTTCTTGTACAAAGT
GGTTGATCTAGAGGGCCCGCGGTTCGAAGGTAAGCCTATCCCTAACCCTCTCCTCGGTCTCGATTC
TACGCGTACCGGTTAGTAATGAGTTTAAACGGGGGAGGCTAACTGAAACACGGAAGGAGACAATA
CCGGAAGGAACCCGCGCTATGACGGCAATAAAAAGACAGAATAAAACGCACGGGTGTTGGGTCG
TTTGTTCATAAACGCGGGGTTCGGTCCCAGGGCTGGCACTCTGTCGATACCCCACCGAGACCCCAT
TGGGGCCAATACGCCCGCGTTTCTTCCTTTTCCCCACCCCACCCCCCAAGTTCGGGTGAAGGCCCA
GGGCTCGCAGCCAACGTCGGGGCGGCAGGCCCTGCCATAGCAGATCCGATTCGACAGATCACTGA
AATGTGTGGGCGTGGCTTAAGGGTGGGAAAGAATATATAAGGTGGGGGTCTTATGTAGTTTTGTAT
CTGTTTTGCAGCAGCCGCCGCCGCCATGAGCACCAACTCGTTTGATGGAAGCATTGTGAGCTCATA
TTTGACAACGCGCATGCCCCCATGGGCCGGGGTGCGTCAGAATGTGATGGGCTCCAGCATTGATG
GTCGCCCCGTCCTGCCCGCAAACTCTACTACCTTGACCTACGAGACCGTGTCTGGAACGCCGTTGG
AGACTGCAGCCTCCGCCGCCGCTTCAGCCGCTGCAGCCACCGCCCGCGGGATTGTGACTGACTTTG
CTTTCCTGAGCCCGCTTGCAAGCAGTGCAGCTTCCCGTTCATCCGCCCGCGATGACAAGTTGACGG
CTCTTTTGGCACAATTGGATTCTTTGACCCGGGAACTTAATGTCGTTTCTCAGCAGCTGTTGGATCT
GCGCCAGCAGGTTTCTGCCCTGAAGGCTTCCTCCCCTCCCAATGCGGTTTAAAACATAAATAAAAA
ACCAGACTCTGTTTGGATTTGGATCAAGCAAGTGTCTTGCTGTCTTTATTTAGGGGTTTTGCGCGCG
CGGTAGGCCCGGGACCAGCGGTCTCGGTCGTTGAGGGTCCTGTGTATTTTTTCCAGGACGTGGTAA
AGGTGACTCTGGATGTTCAGATACATGGGCATAAGCCCGTCTCTGGGGTGGAGGTAGCACCACTG
CAGAGCTTCATGCTGCGGGGTGGTGTTGTAGATGATCCAGTCGTAGCAGGAGCGCTGGGCGTGGT
GCCTAAAAATGTCTTTCAGTAGCAAGCTGATTGCCAGGGGCAGGCCCTTGGTGTAAGTGTTTACAA
AGCGGTTAAGCTGGGATGGGTGCATACGTGGGGATATGAGATGCATCTTGGACTGTATTTTTAGGT
TGGCTATGTTCCCAGCCATATCCCTCCGGGGATTCATGTTGTGCAGAACCACCAGCACAGTGTATC
CGGTGCACTTGGGAAATTTGTCATGTAGCTTAGAAGGAAATGCGTGGAAGAACTTGGAGACGCCC
TTGTGACCTCCAAGATTTTCCATGCATTCGTCCATAATGATGGCAATGGGCCCACGGGCGGCGGCC
TGGGCGAAGATATTTCTGGGATCACTAACGTCATAGTTGTGTTCCAGGATGAGATCGTCATAGGCC
ATTTTTACAAAGCGCGGGCGGAGGGTGCCAGACTGCGGTATAATGGTTCCATCCGGCCCAGGGGC
GTAGTTACCCTCACAGATTTGCATTTCCCACGCTTTGAGTTCAGATGGGGGGATCATGTCTACCTGC
GGGGCGATGAAGAAAACGGTTTCCGGGGTAGGGGAGATCAGCTGGGAAGAAAGCAGGTTCCTGA
GCAGCTGCGACTTACCGCAGCCGGTGGGCCCGTAAATCACACCTATTACCGGCTGCAACTGGTAGT
TAAGAGAGCTGCAGCTGCCGTCATCCCTGAGCAGGGGGGCCACTTCGTTAAGCATGTCCCTGACTC
GCATGTTTTCCCTGACCAAATCCGCCAGAAGGCGCTCGCCGCCCAGCGATAGCAGTTCTTGCAAGG
AAGCAAAGTTTTTCAACGGTTTGAGACCGTCCGCCGTAGGCATGCTTTTGAGCGTTTGACCAAGCA
GTTCCAGGCGGTCCCACAGCTCGGTCACCTGCTCTACGGCATCTCGATCCAGCATATCTCCTCGTTT
CGCGGGTTGGGGCGGCTTTCGCTGTACGGCAGTAGTCGGTGCTCGTCCAGACGGGCCAGGGTCAT
GTCTTTCCACGGGCGCAGGGTCCTCGTCAGCGTAGTCTGGGTCACGGTGAAGGGGTGCGCTCCGGG
CTGCGCGCTGGCCAGGGTGCGCTTGAGGCTGGTCCTGCTGGTGCTGAAGCGCTGCCGGTCTTCGCC
CTGCGCGTCGGCCAGGTAGCATTTGACCATGGTGTCATAGTCCAGCCCCTCCGCGGCGTGGCCCTT
GGCGCGCAGCTTGCCCTTGGAGGAGGCGCCGCACGAGGGGCAGTGCAGACTTTTGAGGGCGTAGA
GCTTGGGCGCGAGAAATACCGATTCCGGGGAGTAGGCATCCGCGCCGCAGGCCCCGCAGACGGTC
TCGCATTCCACGAGCCAGGTGAGCTCTGGCCGTTCGGGGTCAAAAACCAGGTTTCCCCCATGCTTT
TTGATGCGTTTCTTACCTCTGGTTTCCATGAGCCGGTGTCCACGCTCGGTGACGAAAAGGCTGTCC
GTGTCCCCGTATACAGACTTGAGAGGCCTGTCCTCGAGCGGTGTTCCGCGGTCCTCCTCGTATAGA
AACTCGGACCACTCTGAGACAAAGGCTCGCGTCCAGGCCAGCACGAAGGAGGCTAAGTGGGAGG
GGTAGCGGTCGTTGTCCACTAGGGGGTCCACTCGCTCCAGGGTGTGAAGACACATGTCGCCCTCTT
CGGCATCAAGGAAGGTGATTGGTTTGTAGGTGTAGGCCACGTGACCGGGTGTTCCTGAAGGGGGG
CTATAAAAGGGGGTGGGGGCGCGTTCGTCCTCACTCTCTTCCGCATCGCTGTCTGCGAGGGCCAGC
TGTTGGGGTGAGTACTCCCTCTGAAAAGCGGGCATGACTTCTGCGCTAAGATTGTCAGTTTCCAAA
AACGAGGAGGATTTGATATTCACCTGGCCCGCGGTGATGCCTTTGAGGGTGGCCGCATCCATCTGG
TCAGAAAAGACAATCTTTTTGTTGTCAAGCTTGGTGGCAAACGACCCGTAGAGGGCGTTGGACAG
CAACTTGGCGATGGAGCGCAGGGTTTGGTTTTTGTCGCGATCGGCGCGCTCCTTGGCCGCGATGTT
TAGCTGCACGTATTCGCGCGCAACGCACCGCCATTCGGGAAAGACGGTGGTGCGCTCGTCGGGCA
CCAGGTGCACGCGCCAACCGCGGTTGTGCAGGGTGACAAGGTCAACGCTGGTGGCTACCTCTCCG
CGTAGGCGCTCGTTGGTCCAGCAGAGGCGGCCGCCCTTGCGCGAGCAGAATGGCGGTAGGGGGTC
TAGCTGCGTCTCGTCCGGGGGGTCTGCGTCCACGGTAAAGACCCCGGGCAGCAGGCGCGCGTCGA
AGTAGTCTATCTTGCATCCTTGCAAGTCTAGCGCCTGCTGCCATGCGCGGGCGGCAAGCGCGCGCT
CGTATGGGTTGAGTGGGGGACCCCATGGCATGGGGTGGGTGAGCGCGGAGGCGTACATGCCGCAA
ATGTCGTAAACGTAGAGGGGCTCTCTGAGTATTCCAAGATATGTAGGGTAGCATCTTCCACCGCGG
ATGCTGGCGCGCACGTAATCGTATAGTTCGTGCGAGGGAGCGAGGAGGTCGGGACCGAGGTTGCT
ACGGGCGGGCTGCTCTGCTCGGAAGACTATCTGCCTGAAGATGGCATGTGAGTTGGATGATATGGT
TGGACGCTGGAAGACGTTGAAGCTGGCGTCTGTGAGACCTACCGCGTCACGCACGAAGGAGGCGT
AGGAGTCGCGCAGCTTGTTGACCAGCTCGGCGGTGACCTGCACGTCTAGGGCGCAGTAGTCCAGG
GTTTCCTTGATGATGTCATACTTATCCTGTCCCTTTTTTTTCCACAGCTCGCGGTTGAGGACAAACT
CTTCGCGGTCTTTCCAGTACTCTTGGATCGGAAACCCGTCGGCCTCCGAACGGTAAGAGCCTAGCA
TGTAGAACTGGTTGACGGCCTGGTAGGCGCAGCATCCCTTTTCTACGGGTAGCGCGTATGCCTGCG
CGGCCTTCCGGAGCGAGGTGTGGGTGAGCGCAAAGGTGTCCCTGACCATGACCAGCATGAAGGGC
ACGAGCTGCTTCCCAAAGGCCCCCATCCAAGTATAGGTCTCTACATCGTAGGTGACAAAGAGACG
CTCGGTGCGAGGATGCGAGCCGATCGGGAAGAACTGGATCTCCCGCCACCAATTGGAGGAGTGGC
TATTGATGTGGTGAAAGTAGAAGTCCCTGCGACGGGCCGAACACTCGTGCTGGCTTTTGTAAAAAC
GTGCGCAGTACTGGCAGCGGTGCACGGGCTGTACATCCTGCACGAGGTTGACCTGACGACCGCGC
ACAAGGAAGCAGAGTGGGAATTTGAGCCCCTCGCCTGGCGGGTTTGGCTGGTGGTCTTCTACTTCG
GCTGCTTGTCCTTGACCGTCTGGCTGCTCGAGGGGAGTTACGGTGGATCGGACCACCACGCCGCGC
GAGCCCAAAGTCCAGATGTCCGCGCGCGGCGGTCGGAGCTTGATGACAACATCGCGCAGATGGGA
GCTGTCCATGGTCTGGAGCTCCCGCGGCGTCAGGTCAGGCGGGAGCTCCTGCAGGTTTACCTCGCA
TAGACGGGTCAGGGCGCGGGCTAGATCCAGGTGATACCTAATTTCCAGGGGCTGGTTGGTGGCGG
CGTCGATGGCTTGCAAGAGGCCGCATCCCCGCGGCGCGACTACGGTACCGCGCGGCGGGCGGTGG
GCCGCGGGGGTGTCCTTGGATGATGCATCTAAAAGCGGTGACGCGGGCGAGCCCCCGGAGGTAGG
GGGGGCTCCGGACCCGCCGGGAGAGGGGGCAGGGGCACGTCGGCGCCGCGCGCGGGCAGGAGCT
GGTGCTGCGCGCGTAGGTTGCTGGCGAACGCGACGACGCGGCGGTTGATCTCCTGAATCTGGCGC
CTCTGCGTGAAGACGACGGGCCCGGTGAGCTTGAACCTGAAAGAGAGTTCGACAGAATCAATTTC
GGTGTCGTTGACGGCGGCCTGGCGCAAAATCTCCTGCACGTCTCCTGAGTTGTCTTGATAGGCGAT
CTCGGCCATGAACTGCTCGATCTCTTCCTCCTGGAGATCTCCGCGTCCGGCTCGCTCCACGGTGGC
GGCGAGGTCGTTGGAAATGCGGGCCATGAGCTGCGAGAAGGCGTTGAGGCCTCCCTCGTTCCAGA
CGCGGCTGTAGACCACGCCCCCTTCGGCATCGCGGGCGCGCATGACCACCTGCGCGAGATTGAGC
TCCACGTGCCGGGCGAAGACGGCGTAGTTTCGCAGGCGCTGAAAGAGGTAGTTGAGGGTGGTGGC
GGTGTGTTCTGCCACGAAGAAGTACATAACCCAGCGTCGCAACGTGGATTCGTTGATATCCCCCAA
GGCCTCAAGGCGCTCCATGGCCTCGTAGAAGTCCACGGCGAAGTTGAAAAACTGGGAGTTGCGCG
CCGACACGGTTAACTCCTCCTCCAGAAGACGGATGAGCTCGGCGACAGTGTCGCGCACCTCGCGCT
CAAAGGCTACAGGGGCCTCTTCTTCTTCTTCAATCTCCTCTTCCATAAGGGCCTCCCCTTCTTCTTCT
TCTGGCGGCGGTGGGGGAGGGGGGACACGGCGGCGACGACGGCGCACCGGGAGGCGGTCGACAA
AGCGCTCGATCATCTCCCCGCGGCGACGGCGCATGGTCTCGGTGACGGCGCGGCCGTTCTCGCGGG
GGCGCAGTTGGAAGACGCCGCCCGTCATGTCCCGGTTATGGGTTGGCGGGGGGCTGCCATGCGGC
AGGGATACGGCGCTAACGATGCATCTCAACAATTGTTGTGTAGGTACTCCGCCGCCGAGGGACCT
GAGCGAGTCCGCATCGACCGGATCGGAAAACCTCTCGAGAAAGGCGTCTAACCAGTCACAGTCGC
AAGGTAGGCTGAGCACCGTGGCGGGCGGCAGCGGGCGGCGGTCGGGGTTGTTTCTGGCGGAGGTG
CTGCTGATGATGTAATTAAAGTAGGCGGTCTTGAGACGGCGGATGGTCGACAGAAGCACCATGTC
CTTGGGTCCGGCCTGCTGAATGCGCAGGCGGTCGGCCATGCCCCAGGCTTCGTTTTGACATCGGCG
CAGGTCTTTGTAGTAGTCTTGCATGAGCCTTTCTACCGGCACTTCTTCTTCTCCTTCCTCTTGTCCTG
CATCTCTTGCATCTATCGCTGCGGCGGCGGCGGAGTTTGGCCGTAGGTGGCGCCCTCTTCCTCCCAT
GCGTGTGACCCCGAAGCCCCTCATCGGCTGAAGCAGGGCTAGGTCGGCGACAACGCGCTCGGCTA
ATATGGCCTGCTGCACCTGCGTGAGGGTAGACTGGAAGTCATCCATGTCCACAAAGCGGTGGTAT
GCGCCCGTGTTGATGGTGTAAGTGCAGTTGGCCATAACGGACCAGTTAACGGTCTGGTGACCCGGC
TGCGAGAGCTCGGTGTACCTGAGACGCGAGTAAGCCCTCGAGTCAAATACGTAGTCGTTGCAAGT
CCGCACCAGGTACTGGTATCCCACCAAAAAGTGCGGCGGCGGCTGGCGGTAGAGGGGCCAGCGTA
GGGTGGCCGGGGCTCCGGGGGCGAGATCTTCCAACATAAGGCGATGATATCCGTAGATGTACCTG
GACATCCAGGTGATGCCGGCGGCGGTGGTGGAGGCGCGCGGAAAGTCGCGGACGCGGTTCCAGAT
GTTGCGCAGCGGCAAAAAGTGCTCCATGGTCGGGACGCTCTGGCCGGTCAGGCGCGCGCAATCGT
TGACGCTCTAGACCGTGCAAAAGGAGAGCCTGTAAGCGGGCACTCTTCCGTGGTCTGGTGGATAA
ATTCGCAAGGGTATCATGGCGGACGACCGGGGTTCGAGCCCCGTATCCGGCCGTCCGCCGTGATCC
ATGCGGTTACCGCCCGCGTGTCGAACCCAGGTGTGCGACGTCAGACAACGGGGGAGTGCTCCTTTT
GGCTTCCTTCCAGGCGCGGCGGCTGCTGCGCTAGCTTTTTTGGCCACTGGCCGCGCGCAGCGTAAG
CGGTTAGGCTGGAAAGCGAAAGCATTAAGTGGCTCGCTCCCTGTAGCCGGAGGGTTATTTTCCAAG
GGTTGAGTCGCGGGACCCCCGGTTCGAGTCTCGGACCGGCCGGACTGCGGCGAACGGGGGTTTGC
CTCCCCGTCATGCAAGACCCCGCTTGCAAATTCCTCCGGAAACAGGGACGAGCCCCTTTTTTGCTT
TTCCCAGATGCATCCGGTGCTGCGGCAGATGCGCCCCCCTCCTCAGCAGCGGCAAGAGCAAGAGC
AGCGGCAGACATGCAGGGCACCCTCCCCTCCTCCTACCGCGTCAGGAGGGGCGACATCCGCGGTT
GACGCGGCAGCAGATGGTGATTACGAACCCCCGCGGCGCCGGGCCCGGCACTACCTGGACTTGGA
GGAGGGCGAGGGCCTGGCGCGGCTAGGAGCGCCCTCTCCTGAGCGGCACCCAAGGGTGCAGCTGA
AGCGTGATACGCGTGAGGCGTACGTGCCGCGGCAGAACCTGTTTCGCGACCGCGAGGGAGAGGAG
CCCGAGGAGATGCGGGATCGAAAGTTCCACGCAGGGCGCGAGCTGCGGCATGGCCTGAATCGCGA
GCGGTTGCTGCGCGAGGAGGACTTTGAGCCCGACGCGCGAACCGGGATTAGTCCCGCGCGCGCAC
ACGTGGCGGCCGCCGACCTGGTAACCGCATACGAGCAGACGGTGAACCAGGAGATTAACTTTCAA
AAAAGCTTTAACAACCACGTGCGTACGCTTGTGGCGCGCGAGGAGGTGGCTATAGGACTGATGCA
TCTGTGGGACTTTGTAAGCGCGCTGGAGCAAAACCCAAATAGCAAGCCGCTCATGGCGCAGCTGT
TCCTTATAGTGCAGCACAGCAGGGACAACGAGGCATTCAGGGATGCGCTGCTAAACATAGTAGAG
CCCGAGGGCCGCTGGCTGCTCGATTTGATAAACATCCTGCAGAGCATAGTGGTGCAGGAGCGCAG
CTTGAGCCTGGCTGACAAGGTGGCCGCCATCAACTATTCCATGCTTAGCCTGGGCAAGTTTTACGC
CCGCAAGATATACCATACCCCTTACGTTCCCATAGACAAGGAGGTAAAGATCGAGGGGTTCTACA
TGCGCATGGCGCTGAAGGTGCTTACCTTGAGCGACGACCTGGGCGTTTATCGCAACGAGCGCATCC
ACAAGGCCGTGAGCGTGAGCCGGCGGCGCGAGCTCAGCGACCGCGAGCTGATGCACAGCCTGCAA
AGGGCCCTGGCTGGCACGGGCAGCGGCGATAGAGAGGCCGAGTCCTACTTTGACGCGGGCGCTGA
CCTGCGCTGGGCCCCAAGCCGACGCGCCCTGGAGGCAGCTGGGGCCGGACCTGGGCTGGCGGTGG
CACCCGCGCGCGCTGGCAACGTCGGCGGCGTGGAGGAATATGACGAGGACGATGAGTACGAGCC
AGAGGACGGCGAGTACTAAGCGGTGATGTTTCTGATCAGATGATGCAAGACGCAACGGACCCGGC
GGTGCGGGCGGCGCTGCAGAGCCAGCCGTCCGGCCTTAACTCCACGGACGACTGGCGCCAGGTCA
TGGACCGCATCATGTCGCTGACTGCGCGCAATCCTGACGCGTTCCGGCAGCAGCCGCAGGCCAAC
CGGCTCTCCGCAATTCTGGAAGCGGTGGTCCCGGCGCGCGCAAACCCCACGCACGAGAAGGTGCT
GGCGATCGTAAACGCGCTGGCCGAAAACAGGGCCATCCGGCCCGACGAGGCCGGCCTGGTCTACG
ACGCGCTGCTTCAGCGCGTGGCTCGTTACAACAGCGGCAACGTGCAGACCAACCTGGACCGGCTG
GTGGGGGATGTGCGCGAGGCCGTGGCGCAGCGTGAGCGCGCGCAGCAGCAGGGCAACCTGGGCT
CCATGGTTGCACTAAACGCCTTCCTGAGTACACAGCCCGCCAACGTGCCGCGGGGACAGGAGGAC
TACACCAACTTTGTGAGCGCACTGCGGCTAATGGTGACTGAGACACCGCAAAGTGAGGTGTACCA
GTCTGGGCCAGACTATTTTTTCCAGACCAGTAGACAAGGCCTGCAGACCGTAAACCTGAGCCAGG
CTTTCAAAAACTTGCAGGGGCTGTGGGGGGTGCGGGCTCCCACAGGCGACCGCGCGACCGTGTCT
AGCTTGCTGACGCCCAACTCGCGCCTGTTGCTGCTGCTAATAGCGCCCTTCACGGACAGTGGCAGC
GTGTCCCGGGACACATACCTAGGTCACTTGCTGACACTGTACCGCGAGGCCATAGGTCAGGCGCAT
GTGGACGAGCATACTTTCCAGGAGATTACAAGTGTCAGCCGCGCGCTGGGGCAGGAGGACACGGG
CAGCCTGGAGGCAACCCTAAACTACCTGCTGACCAACCGGCGGCAGAAGATCCCCTCGTTGCACA
GTTTAAACAGCGAGGAGGAGCGCATTTTGCGCTACGTGCAGCAGAGCGTGAGCCTTAACCTGATG
CGCGACGGGGTAACGCCCAGCGTGGCGCTGGACATGACCGCGCGCAACATGGAACCGGGCATGTA
TGCCTCAAACCGGCCGTTTATCAACCGCCTAATGGACTACTTGCATCGCGCGGCCGCCGTGAACCC
CGAGTATTTCACCAATGCCATCTTGAACCCGCACTGGCTACCGCCCCCTGGTTTCTACACCGGGGG
ATTCGAGGTGCCCGAGGGTAACGATGGATTCCTCTGGGACGACATAGACGACAGCGTGTTTTCCCC
GCAACCGCAGACCCTGCTAGAGTTGCAACAGCGCGAGCAGGCAGAGGCGGCGCTGCGAAAGGAA
AGCTTCCGCAGGCCAAGCAGCTTGTCCGATCTAGGCGCTGCGGCCCCGCGGTCAGATGCTAGTAGC
CCATTTCCAAGCTTGATAGGGTCTCTTACCAGCACTCGCACCACCCGCCCGCGCCTGCTGGGCGAG
GAGGAGTACCTAAACAACTCGCTGCTGCAGCCGCAGCGCGAAAAAAACCTGCCTCCGGCATTTCC
CAACAACGGGATAGAGAGCCTAGTGGACAAGATGAGTAGATGGAAGACGTACGCGCAGGAGCAC
AGGGACGTGCCAGGCCCGCGCCCGCCCACCCGTCGTCAAAGGCACGACCGTCAGCGGGGTCTGGT
GTGGGAGGACGATGACTCGGCAGACGACAGCAGCGTCCTGGATTTGGGAGGGAGTGGCAACCCGT
TTGCGCACCTTCGCCCCAGGCTGGGGAGAATGTTTTAAAAAAAAAAAAAGCATGATGCAAAATAA
AAAACTCACCAAGGCCATGGCACCGAGCGTTGGTTTTCTTGTATTCCCCTTAGTATGCGGCGCGCG
GCGATGTATGAGGAAGGTCCTCCTCCCTCCTACGAGAGTGTGGTGAGCGCGGCGCCAGTGGCGGC
GGCGCTGGGTTCTCCCTTCGATGCTCCCCTGGACCCGCCGTTTGTGCCTCCGCGGTACCTGCGGCCT
ACCGGGGGGAGAAACAGCATCCGTTACTCTGAGTTGGCACCCCTATTCGACACCACCCGTGTGTAC
CTGGTGGACAACAAGTCAACGGATGTGGCATCCCTGAACTACCAGAACGACCACAGCAACTTTCT
GACCACGGTCATTCAAAACAATGACTACAGCCCGGGGGAGGCAAGCACACAGACCATCAATCTTG
ACGACCGGTCGCACTGGGGCGGCGACCTGAAAACCATCCTGCATACCAACATGCCAAATGTGAAC
GAGTTCATGTTTACCAATAAGTTTAAGGCGCGGGTGATGGTGTCGCGCTTGCCTACTAAGGACAAT
CAGGTGGAGCTGAAATACGAGTGGGTGGAGTTCACGCTGCCCGAGGGCAACTACTCCGAGACCAT
GACCATAGACCTTATGAACAACGCGATCGTGGAGCACTACTTGAAAGTGGGCAGACAGAACGGGG
TTCTGGAAAGCGACATCGGGGTAAAGTTTGACACCCGCAACTTCAGACTGGGGTTTGACCCCGTCA
CTGGTCTTGTCATGCCTGGGGTATATACAAACGAAGCCTTCCATCCAGACATCATTTTGCTGCCAG
GATGCGGGGTGGACTTCACCCACAGCCGCCTGAGCAACTTGTTGGGCATCCGCAAGCGGCAACCC
TTCCAGGAGGGCTTTAGGATCACCTACGATGATCTGGAGGGTGGTAACATTCCCGCACTGTTGGAT
GTGGACGCCTACCAGGCGAGCTTGAAAGATGACACCGAACAGGGCGGGGGTGGCGCAGGCGGCA
GCAACAGCAGTGGCAGCGGCGCGGAAGAGAACTCCAACGCGGCAGCCGCGGCAATGCAGCCGGT
GGAGGACATGAACGATCATGCCATTCGCGGCGACACCTTTGCCACACGGGCTGAGGAGAAGCGCG
CTGAGGCCGAAGCAGCGGCCGAAGCTGCCGCCCCCGCTGCGCAACCCGAGGTCGAGAAGCCTCAG
AAGAAACCGGTGATCAAACCCCTGACAGAGGACAGCAAGAAACGCAGTTACAACCTAATAAGCA
ATGACAGCACCTTCACCCAGTACCGCAGCTGGTACCTTGCATACAACTACGGCGACCCTCAGACCG
GAATCCGCTCATGGACCCTGCTTTGCACTCCTGACGTAACCTGCGGCTCGGAGCAGGTCTACTGGT
CGTTGCCAGACATGATGCAAGACCCCGTGACCTTCCGCTCCACGCGCCAGATCAGCAACTTTCCGG
TGGTGGGCGCCGAGCTGTTGCCCGTGCACTCCAAGAGCTTCTACAACGACCAGGCCGTCTACTCCC
AACTCATCCGCCAGTTTACCTCTCTGACCCACGTGTTCAATCGCTTTCCCGAGAACCAGATTTTGGC
GCGCCCGCCAGCCCCCACCATCACCACCGTCAGTGAAAACGTTCCTGCTCTCACAGATCACGGGAC
GCTACCGCTGCGCAACAGCATCGGAGGAGTCCAGCGAGTGACCATTACTGACGCCAGACGCCGCA
CCTGCCCCTACGTTTACAAGGCCCTGGGCATAGTCTCGCCGCGCGTCCTATCGAGCCGCACTTTTTG
AGCAAGCATGTCCATCCTTATATCGCCCAGCAATAACACAGGCTGGGGCCTGCGCTTCCCAAGCAA
GATGTTTGGCGGGGCCAAGAAGCGCTCCGACCAACACCCAGTGCGCGTGCGCGGGCACTACCGCG
CGCCCTGGGGCGCGCACAAACGCGGCCGCACTGGGCGCACCACCGTCGATGACGCCATCGACGCG
GTGGTGGAGGAGGCGCGCAACTACACGCCCACGCCGCCACCAGTGTCCACAGTGGACGCGGCCAT
TCAGACCGTGGTGCGCGGAGCCCGGCGCTATGCTAAAATGAAGAGACGGCGGAGGCGCGTAGCAC
GTCGCCACCGCCGCCGACCCGGCACTGCCGCCCAACGCGCGGCGGCGGCCCTGCTTAACCGCGCA
CGTCGCACCGGCCGACGGGCGGCCATGCGGGCCGCTCGAAGGCTGGCCGCGGGTATTGTCACTGT
GCCCCCCAGGTCCAGGCGACGAGCGGCCGCCGCAGCAGCCGCGGCCATTAGTGCTATGACTCAGG
GTCGCAGGGGCAACGTGTATTGGGTGCGCGACTCGGTTAGCGGCCTGCGCGTGCCCGTGCGCACC
CGCCCCCCGCGCAACTAGATTGCAAGAAAAAACTACTTAGACTCGTACTGTTGTATGTATCCAGCG
GCGGCGGCGCGCAACGAAGCTATGTCCAAGCGCAAAATCAAAGAAGAGATGCTCCAGGTCATCGC
GCCGGAGATCTATGGCCCCCCGAAGAAGGAAGAGCAGGATTACAAGCCCCGAAAGCTAAAGCGG
GTCAAAAAGAAAAAGAAAGATGATGATGATGAACTTGACGACGAGGTGGAACTGCTGCACGCTA
CCGCGCCCAGGCGACGGGTACAGTGGAAAGGTCGACGCGTAAAACGTGTTTTGCGACCCGGCACC
ACCGTAGTCTTTACGCCCGGTGAGCGCTCCACCCGCACCTACAAGCGCGTGTATGATGAGGTGTAC
GGCGACGAGGACCTGCTTGAGCAGGCCAACGAGCGCCTCGGGGAGTTTGCCTACGGAAAGCGGCA
TAAGGACATGCTGGCGTTGCCGCTGGACGAGGGCAACCCAACACCTAGCCTAAAGCCCGTAACAC
TGCAGCAGGTGCTGCCCGCGCTTGCACCGTCCGAAGAAAAGCGCGGCCTAAAGCGCGAGTCTGGT
GACTTGGCACCCACCGTGCAGCTGATGGTACCCAAGCGCCAGCGACTGGAAGATGTCTTGGAAAA
AATGACCGTGGAACCTGGGCTGGAGCCCGAGGTCCGCGTGCGGCCAATCAAGCAGGTGGCGCCGG
GACTGGGCGTGCAGACCGTGGACGTTCAGATACCCACTACCAGTAGCACCAGTATTGCCACCGCC
ACAGAGGGCATGGAGACACAAACGTCCCCGGTTGCCTCAGCGGTGGCGGATGCCGCGGTGCAGGC
GGTCGCTGCGGCCGCGTCCAAGACCTCTACGGAGGTGCAAACGGACCCGTGGATGTTTCGCGTTTC
AGCCCCCCGGCGCCCGCGCCGTTCGAGGAAGTACGGCGCCGCCAGCGCGCTACTGCCCGAATATG
CCCTACATCCTTCCATTGCGCCTACCCCCGGCTATCGTGGCTACACCTACCGCCCCAGAAGACGAG
CAACTACCCGACGCCGAACCACCACTGGAACCCGCCGCCGCCGTCGCCGTCGCCAGCCCGTGCTG
GCCCCGATTTCCGTGCGCAGGGTGGCTCGCGAAGGAGGCAGGACCCTGGTGCTGCCAACAGCGCG
CTACCACCCCAGCATCGTTTAAAAGCCGGTCTTTGTGGTTCTTGCAGATATGGCCCTCACCTGCCGC
CTCCGTTTCCCGGTGCCGGGATTCCGAGGAAGAATGCACCGTAGGAGGGGCATGGCCGGCCACGG
CCTGACGGGCGGCATGCGTCGTGCGCACCACCGGCGGCGGCGCGCGTCGCACCGTCGCATGCGCG
GCGGTATCCTGCCCCTCCTTATTCCACTGATCGCCGCGGCGATTGGCGCCGTGCCCGGAATTGCAT
CCGTGGCCTTGCAGGCGCAGAGACACTGATTAAAAACAAGTTGCATGTGGAAAAATCAAAATAAA
AAGTCTGGACTCTCACGCTCGCTTGGTCCTGTAACTATTTTGTAGAATGGAAGACATCAACTTTGC
GTCTCTGGCCCCGCGACACGGCTCGCGCCCGTTCATGGGAAACTGGCAAGATATCGGCACCAGCA
ATATGAGCGGTGGCGCCTTCAGCTGGGGCTCGCTGTGGAGCGGCATTAAAAATTTCGGTTCCACCG
TTAAGAACTATGGCAGCAAGGCCTGGAACAGCAGCACAGGCCAGATGCTGAGGGATAAGTTGAA
AGAGCAAAATTTCCAACAAAAGGTGGTAGATGGCCTGGCCTCTGGCATTAGCGGGGTGGTGGACC
TGGCCAACCAGGCAGTGCAAAATAAGATTAACAGTAAGCTTGATCCCCGCCCTCCCGTAGAGGAG
CCTCCACCGGCCGTGGAGACAGTGTCTCCAGAGGGGCGTGGCGAAAAGCGTCCGCGCCCCGACAG
GGAAGAAACTCTGGTGACGCAAATAGACGAGCCTCCCTCGTACGAGGAGGCACTAAAGCAAGGCC
TGCCCACCACCCGTCCCATCGCGCCCATGGCTACCGGAGTGCTGGGCCAGCACACACCCGTAACGC
TGGACCTGCCTCCCCCCGCCGACACCCAGCAGAAACCTGTGCTGCCAGGCCCGACCGCCGTTGTTG
TAACCCGTCCTAGCCGCGCGTCCCTGCGCCGCGCCGCCAGCGGTCCGCGATCGTTGCGGCCCGTAG
CCAGTGGCAACTGGCAAAGCACACTGAACAGCATCGTGGGTCTGGGGGTGCAATCCCTGAAGCGC
CGACGATGCTTCTGATAGCTAACGTGTCGTATGTGTGTCATGTATGCGTCCATGTCGCCGCCAGAG
GAGCTGCTGAGCCGCCGCGCGCCCGCTTTCCAAGATGGCTACCCCTTCGATGATGCCGCAGTGGTC
TTACATGCACATCTCGGGCCAGGACGCCTCGGAGTACCTGAGCCCCGGGCTGGTGCAGTTTGCCCG
CGCCACCGAGACGTACTTCAGCCTGAATAACAAGTTTAGAAACCCCACGGTGGCGCCTACGCACG
ACGTGACCACAGACCGGTCCCAGCGTTTGACGCTGCGGTTCATCCCTGTGGACCGTGAGGATACTG
CGTACTCGTACAAGGCGCGGTTCACCCTAGCTGTGGGTGATAACCGTGTGCTGGACATGGCTTCCA
CGTACTTTGACATCCGCGGCGTGCTGGACAGGGGCCCTACTTTTAAGCCCTACTCTGGCACTGCCT
ACAACGCCCTGGCTCCCAAGGGTGCCCCAAATCCTTGCGAATGGGATGAAGCTGCTACTGCTCTTG
AAATAAACCTAGAAGAAGAGGACGATGACAACGAAGACGAAGTAGACGAGCAAGCTGAGCAGCA
AAAAACTCACGTATTTGGGCAGGCGCCTTATTCTGGTATAAATATTACAAAGGAGGGTATTCAAAT
AGGTGTCGAAGGTCAAACACCTAAATATGCCGATAAAACATTTCAACCTGAACCTCAAATAGGAG
AATCTCAGTGGTACGAAACAGAAATTAATCATGCAGCTGGGAGAGTCCTAAAAAAGACTACCCCA
ATGAAACCATGTTACGGTTCATATGCAAAACCCACAAATGAAAATGGAGGGCAAGGCATTCTTGT
AAAGCAACAAAATGGAAAGCTAGAAAGTCAAGTGGAAATGCAATTTTTCTCAACTACTGAGGCAG
CCGCAGGCAATGGTGATAACTTGACTCCTAAAGTGGTATTGTACAGTGAAGATGTAGATATAGAA
ACCCCAGACACTCATATTTCTTACATGCCCACTATTAAGGAAGGTAACTCACGAGAACTAATGGGC
CAACAATCTATGCCCAACAGGCCTAATTACATTGCTTTTAGGGACAATTTTATTGGTCTAATGTATT
ACAACAGCACGGGTAATATGGGTGTTCTGGCGGGCCAAGCATCGCAGTTGAATGCTGTTGTAGATT
TGCAAGACAGAAACACAGAGCTTTCATACCAGCTTTTGCTTGATTCCATTGGTGATAGAACCAGGT
ACTTTTCTATGTGGAATCAGGCTGTTGACAGCTATGATCCAGATGTTAGAATTATTGAAAATCATG
GAACTGAAGATGAACTTCCAAATTACTGCTTTCCACTGGGAGGTGTGATTAATACAGAGACTCTTA
CCAAGGTAAAACCTAAAACAGGTCAGGAAAATGGATGGGAAAAAGATGCTACAGAATTTTCAGAT
AAAAATGAAATAAGAGTTGGAAATAATTTTGCCATGGAAATCAATCTAAATGCCAACCTGTGGAG
AAATTTCCTGTACTCCAACATAGCGCTGTATTTGCCCGACAAGCTAAAGTACAGTCCTTCCAACGT
AAAAATTTCTGATAACCCAAACACCTACGACTACATGAACAAGCGAGTGGTGGCTCCCGGGCTAG
TGGACTGCTACATTAACCTTGGAGCACGCTGGTCCCTTGACTATATGGACAACGTCAACCCATTTA
ACCACCACCGCAATGCTGGCCTGCGCTACCGCTCAATGTTGCTGGGCAATGGTCGCTATGTGCCCT
TCCACATCCAGGTGCCTCAGAAGTTCTTTGCCATTAAAAACCTCCTTCTCCTGCCGGGCTCATACAC
CTACGAGTGGAACTTCAGGAAGGATGTTAACATGGTTCTGCAGAGCTCCCTAGGAAATGACCTAA
GGGTTGACGGAGCCAGCATTAAGTTTGATAGCATTTGCCTTTACGCCACCTTCTTCCCCATGGCCC
ACAACACCGCCTCCACGCTTGAGGCCATGCTTAGAAACGACACCAACGACCAGTCCTTTAACGACT
ATCTCTCCGCCGCCAACATGCTCTACCCTATACCCGCCAACGCTACCAACGTGCCCATATCCATCC
CCTCCCGCAACTGGGCGGCTTTCCGCGGCTGGGCCTTCACGCGCCTTAAGACTAAGGAAACCCCAT
CACTGGGCTCGGGCTACGACCCTTATTACACCTACTCTGGCTCTATACCCTACCTAGATGGAACCTT
TTACCTCAACCACACCTTTAAGAAGGTGGCCATTACCTTTGACTCTTCTGTCAGCTGGCCTGGCAAT
GACCGCCTGCTTACCCCCAACGAGTTTGAAATTAAGCGCTCAGTTGACGGGGAGGGTTACAACGTT
GCCCAGTGTAACATGACCAAAGACTGGTTCCTGGTACAAATGCTAGCTAACTATAACATTGGCTAC
CAGGGCTTCTATATCCCAGAGAGCTACAAGGACCGCATGTACTCCTTCTTTAGAAACTTCCAGCCC
ATGAGCCGTCAGGTGGTGGATGATACTAAATACAAGGACTACCAACAGGTGGGCATCCTACACCA
ACACAACAACTCTGGATTTGTTGGCTACCTTGCCCCCACCATGCGCGAAGGACAGGCCTACCCTGC
TAACTTCCCCTATCCGCTTATAGGCAAGACCGCAGTTGACAGCATTACCCAGAAAAAGTTTCTTTG
CGATCGCACCCTTTGGCGCATCCCATTCTCCAGTAACTTTATGTCCATGGGCGCACTCACAGACCT
GGGCCAAAACCTTCTCTACGCCAACTCCGCCCACGCGCTAGACATGACTTTTGAGGTGGATCCCAT
GGACGAGCCCACCCTTCTTTATGTTTTGTTTGAAGTCTTTGACGTGGTCCGTGTGCACCAGCCGCAC
CGCGGCGTCATCGAAACCGTGTACCTGCGCACGCCCTTCTCGGCCGGCAACGCCACAACATAAAG
AAGCAAGCAACATCAACAACAGCTGCCGCCATGGGCTCCAGTGAGCAGGAACTGAAAGCCATTGT
CAAAGATCTTGGTTGTGGGCCATATTTTTTGGGCACCTATGACAAGCGCTTTCCAGGCTTTGTTTCT
CCACACAAGCTCGCCTGCGCCATAGTCAATACGGCCGGTCGCGAGACTGGGGGCGTACACTGGAT
GGCCTTTGCCTGGAACCCGCACTCAAAAACATGCTACCTCTTTGAGCCCTTTGGCTTTTCTGACCAG
CGACTCAAGCAGGTTTACCAGTTTGAGTACGAGTCACTCCTGCGCCGTAGCGCCATTGCTTCTTCC
CCCGACCGCTGTATAACGCTGGAAAAGTCCACCCAAAGCGTACAGGGGCCCAACTCGGCCGCCTG
TGGACTATTCTGCTGCATGTTTCTCCACGCCTTTGCCAACTGGCCCCAAACTCCCATGGATCACAAC
CCCACCATGAACCTTATTACCGGGGTACCCAACTCCATGCTCAACAGTCCCCAGGTACAGCCCACC
CTGCGTCGCAACCAGGAACAGCTCTACAGCTTCCTGGAGCGCCACTCGCCCTACTTCCGCAGCCAC
AGTGCGCAGATTAGGAGCGCCACTTCTTTTTGTCACTTGAAAAACATGTAAAAATAATGTACTAGA
GACACTTTCAATAAAGGCAAATGCTTTTATTTGTACACTCTCGGGTGATTATTTACCCCCACCCTTG
CCGTCTGCGCCGTTTAAAAATCAAAGGGGTTCTGCCGCGCATCGCTATGCGCCACTGGCAGGGACA
CGTTGCGATACTGGTGTTTAGTGCTCCACTTAAACTCAGGCACAACCATCCGCGGCAGCTCGGTGA
AGTTTTCACTCCACAGGCTGCGCACCATCACCAACGCGTTTAGCAGGTCGGGCGCCGATATCTTGA
AGTCGCAGTTGGGGCCTCCGCCCTGCGCGCGCGAGTTGCGATACACAGGGTTGCAGCACTGGAAC
ACTATCAGCGCCGGGTGGTGCACGCTGGCCAGCACGCTCTTGTCGGAGATCAGATCCGCGTCCAG
GTCCTCCGCGTTGCTCAGGGCGAACGGAGTCAACTTTGGTAGCTGCCTTCCCAAAAAGGGCGCGTG
CCCAGGCTTTGAGTTGCACTCGCACCGTAGTGGCATCAAAAGGTGACCGTGCCCGGTCTGGGCGTT
AGGATACAGCGCCTGCATAAAAGCCTTGATCTGCTTAAAAGCCACCTGAGCCTTTGCGCCTTCAGA
GAAGAACATGCCGCAAGACTTGCCGGAAAACTGATTGGCCGGACAGGCCGCGTCGTGCACGCAGC
ACCTTGCGTCGGTGTTGGAGATCTGCACCACATTTCGGCCCCACCGGTTCTTCACGATCTTGGCCTT
GCTAGACTGCTCCTTCAGCGCGCGCTGCCCGTTTTCGCTCGTCACATCCATTTCAATCACGTGCTCC
TTATTTATCATAATGCTTCCGTGTAGACACTTAAGCTCGCCTTCGATCTCAGCGCAGCGGTGCAGCC
ACAACGCGCAGCCCGTGGGCTCGTGATGCTTGTAGGTCACCTCTGCAAACGACTGCAGGTACGCCT
GCAGGAATCGCCCCATCATCGTCACAAAGGTCTTGTTGCTGGTGAAGGTCAGCTGCAACCCGCGGT
GCTCCTCGTTCAGCCAGGTCTTGCATACGGCCGCCAGAGCTTCCACTTGGTCAGGCAGTAGTTTGA
AGTTCGCCTTTAGATCGTTATCCACGTGGTACTTGTCCATCAGCGCGCGCGCAGCCTCCATGCCCTT
CTCCCACGCAGACACGATCGGCACACTCAGCGGGTTCATCACCGTAATTTCACTTTCCGCTTCGCT
GGGCTCTTCCTCTTCCTCTTGCGTCCGCATACCACGCGCCACTGGGTCGTCTTCATTCAGCCGCCGC
ACTGTGCGCTTACCTCCTTTGCCATGCTTGATTAGCACCGGTGGGTTGCTGAAACCCACCATTTGTA
GCGCCACATCTTCTCTTTCTTCCTCGCTGTCCACGATTACTTGACAATTAATCATCGGCTCGTATAA
TGATGCAGTACATTTTCACAGGAGGTACAGCTATGACCATGATTACGGATTCACTGGCCGTCGTTT
TACAACGTCGTGACTGGGAAAACCCTGGCGTTACCCAACTTAATCGCCTTGCAGCACATCCCCCTT
TCGCCAGCTGGCGTAATAGCGAAGAGGCCCGCACCGATCGCCCTTCCCAACAGTTGCGCAGCCTG
AATGGCGAATAGGTCGCGCCGCACCGCGTCCGCGCTCGGGGGTGGTTTCGCGCTGCTCCTCTTCCC
GACTGGCCATTTCCTTCTCCTATAGGCAGAAAAAGATCATGGAGTCAGTCGAGAAGAAGGACAGC
CTAACCGCCCCCTCTGAGTTCGCCACCACCGCCTCCACCGATGCCGCCAACGCGCCTACCACCTTC
CCCGTCGAGGCACCCCCGCTTGAGGAGGAGGAAGTGATTATCGAGCAGGACCCAGGTTTTGTAAG
CGAAGACGACGAGGACCGCTCAGTACCAACAGAGGATAAAAAGCAAGACCAGGACAACGCAGAG
GCAAACGAGGAACAAGTCGGGCGGGGGGACGAAAGGCATGGCGACTACCTAGATGTGGGAGACG
ACGTGCTGTTGAAGCATCTGCAGCGCCAGTGCGCCATTATCTGCGACGCGTTGCAAGAGCGCAGC
GATGTGCCCCTCGCCATAGCGGATGTCAGCCTTGCCTACGAACGCCACCTATTCTCACCGCGCGTA
CCCCCCAAACGCCAAGAAAACGGCACATGCGAGCCCAACCCGCGCCTCAACTTCTACCCCGTATTT
GCCGTGCCAGAGGTGCTTGCCACCTATCACATCTTTTTCCAAAACTGCAAGATACCCCTATCCTGC
CGTGCCAACCGCAGCCGAGCGGACAAGCAGCTGGCCTTGCGGCAGGGCGCTGTCATACCTGATAT
CGCCTCGCTCAACGAAGTGCCAAAAATCTTTGAGGGTCTTGGACGCGACGAGAAGCGCGCGGCAA
ACGCTCTGCAACAGGAAAACAGCGAAAATGAAAGTCACTCTGGAGTGTTGGTGGAACTCGAGGGT
GACAACGCGCGCCTAGCCGTACTAAAACGCAGCATCGAGGTCACCCACTTTGCCTACCCGGCACTT
AACCTACCCCCCAAGGTCATGAGCACAGTCATGAGTGAGCTGATCGTGCGCCGTGCGCAGCCCCT
GGAGAGGGATGCAAATTTGCAAGAACAAACAGAGGAGGGCCTACCCGCAGTTGGCGACGAGCAG
CTAGCGCGCTGGCTTCAAACGCGCGAGCCTGCCGACTTGGAGGAGCGACGCAAACTAATGATGGC
CGCAGTGCTCGTTACCGTGGAGCTTGAGTGCATGCAGCGGTTCTTTGCTGACCCGGAGATGCAGCG
CAAGCTAGAGGAAACATTGCACTACACCTTTCGACAGGGCTACGTACGCCAGGCCTGCAAGATCT
CCAACGTGGAGCTCTGCAACCTGGTCTCCTACCTTGGAATTTTGCACGAAAACCGCCTTGGGCAAA
ACGTGCTTCATTCCACGCTCAAGGGCGAGGCGCGCCGCGACTACGTCCGCGACTGCGTTTACTTAT
TTCTATGCTACACCTGGCAGACGGCCATGGGCGTTTGGCAGCAGTGCTTGGAGGAGTGCAACCTCA
AGGAGCTGCAGAAACTGCTAAAGCAAAACTTGAAGGACCTATGGACGGCCTTCAACGAGCGCTCC
GTGGCCGCGCACCTGGCGGACATCATTTTCCCCGAACGCCTGCTTAAAACCCTGCAACAGGGTCTG
CCAGACTTCACCAGTCAAAGCATGTTGCAGAACTTTAGGAACTTTATCCTAGAGCGCTCAGGAATC
TTGCCCGCCACCTGCTGTGCACTTCCTAGCGACTTTGTGCCCATTAAGTACCGCGAATGCCCTCCGC
CGCTTTGGGGCCACTGCTACCTTCTGCAGCTAGCCAACTACCTTGCCTACCACTCTGACATAATGG
AAGACGTGAGCGGTGACGGTCTACTGGAGTGTCACTGTCGCTGCAACCTATGCACCCCGCACCGCT
CCCTGGTTTGCAATTCGCAGCTGCTTAACGAAAGTCAAATTATCGGTACCTTTGAGCTGCAGGGTC
CCTCGCCTGACGAAAAGTCCGCGGCTCCGGGGTTGAAACTCACTCCGGGGCTGTGGACGTCGGCTT
ACCTTCGCAAATTTGTACCTGAGGACTACCACGCCCACGAGATTAGGTTCTACGAAGACCAATCCC
GCCCGCCTAATGCGGAGCTTACCGCCTGCGTCATTACCCAGGGCCACATTCTTGGCCAATTGCAAG
CCATCAACAAAGCCCGCCAAGAGTTTCTGCTACGAAAGGGACGGGGGGTTTACTTGGACCCCCAG
TCCGGCGAGGAGCTCAACCCAATCCCCCCGCCGCCGCAGCCCTATCAGCAGCAGCCGCGGGCCCT
TGCTTCCCAGGATGGCACCCAAAAAGAAGCTGCAGCTGCCGCCGCCACCCACGGACGAGGAGGAA
TACTGGGACAGTCAGGCAGAGGAGGTTTTGGACGAGGAGGAGGAGGACATGATGGAAGACTGGG
AGAGCCTAGACGAGGAAGCTTCCGAGGTCGAAGAGGTGTCAGACGAAACACCGTCACCCTCGGTC
GCATTCCCCTCGCCGGCGCCCCAGAAATCGGCAACCGGTTCCAGCATGGCTACAACCTCCGCTCCT
CAGGCGCCGCCGGCACTGCCCGTTCGCCGACCCAACCGTAGATGGGACACCACTGGAACCAGGGC
CGGTAAGTCCAAGCAGCCGCCGCCGTTAGCCCAAGAGCAACAACAGCGCCAAGGCTACCGCTCAT
GGCGCGGGCACAAGAACGCCATAGTTGCTTGCTTGCAAGACTGTGGGGGCAACATCTCCTTCGCCC
GCCGCTTTCTTCTCTACCATCACGGCGTGGCCTTCCCCCGTAACATCCTGCATTACTACCGTCATCT
CTACAGCCCATACTGCACCGGCGGCAGCGGCAGCAACAGCAGCGGCCACACAGAAGCAAAGGCG
ACCGGATAGCAAGACTCTGACAAAGCCCAAGAAATCCACAGCGGCGGCAGCAGCAGGAGGAGGA
GCGCTGCGTCTGGCGCCCAACGAACCCGTATCGACCCGCGAGCTTAGAAACAGGATTTTTCCCACT
CTGTATGCTATATTTCAACAGAGCAGGGGCCAAGAACAAGAGCTGAAAATAAAAAACAGGTCTCT
GCGATCCCTCACCCGCAGCTGCCTGTATCACAAAAGCGAAGATCAGCTTCGGCGCACGCTGGAAG
ACGCGGAGGCTCTCTTCAGTAAATACTGCGCGCTGACTCTTAAGGACTAGTTTCGCGCCCTTTCTC
AAATTTAAGCGCGAAAACTACGTCATCTCCAGCGGCCACACCCGGCGCCAGCACCTGTTGTCAGC
GCCATTATGAGCAAGGAAATTCCCACGCCCTACATGTGGAGTTACCAGCCACAAATGGGACTTGC
GGCTGGAGCTGCCCAAGACTACTCAACCCGAATAAACTACATGAGCGCGGGACCCCACATGATAT
CCCGGGTCAACGGAATACGCGCCCACCGAAACCGAATTCTCCTGGAACAGGCGGCTATTACCACC
ACACCTCGTAATAACCTTAATCCCCGTAGTTGGCCCGCTGCCCTGGTGTACCAGGAAAGTCCCGCT
CCCACCACTGTGGTACTTCCCAGAGACGCCCAGGCCGAAGTTCAGATGACTAACTCAGGGGCGCA
GCTTGCGGGCGGCTTTCGTCACAGGGTGCGGTCGCCCGGGCAGGGTATAACTCACCTGACAATCA
GAGGGCGAGGTATTCAGCTCAACGACGAGTCGGTGAGCTCCTCGCTTGGTCTCCGTCCGGACGGG
ACATTTCAGATCGGCGGCGCCGGCCGCTCTTCATTCACGCCTCGTCAGGCAATCCTAACTCTGCAG
ACCTCGTCCTCTGAGCCGCGCTCTGGAGGCATTGGAACTCTGCAATTTATTGAGGAGTTTGTGCCA
TCGGTCTACTTTAACCCCTTCTCGGGACCTCCCGGCCACTATCCGGATCAATTTATTCCTAACTTTG
ACGCGGTAAAGGACTCGGCGGACGGCTACGACTGAATGTTAAGTGGAGAGGCAGAGCAACTGCG
CCTGAAACACCTGGTCCACTGTCGCCGCCACAAGTGCTTTGCCCGCGACTCCGGTGAGTTTTGCTA
CTTTGAATTGCCCGAGGATCATATCGAGGGCCCGGCGCACGGCGTCCGGCTTACCGCCCAGGGAG
AGCTTGCCCGTAGCCTGATTCGGGAGTTTACCCAGCGCCCCCTGCTAGTTGAGCGGGACAGGGGAC
CCTGTGTTCTCACTGTGATTTGCAACTGTCCTAACCCTGGATTACATCAAGATCTTTGTTGCCATCT
CTGTGCTGAGTATAATAAATACAGAAATTAAAATATACTGGGGCTCCTATCGCCATCCTGTAAACG
CCACCGTCTTCACCCGCCCAAGCAAACCAAGGCGAACCTTACCTGGTACTTTTAACATCTCTCCCT
CTGTGATTTACAACAGTTTCAACCCAGACGGAGTGAGTCTACGAGAGAACCTCTCCGAGCTCAGCT
ACTCCATCAGAAAAAACACCACCCTCCTTACCTGCCGGGAACGTACGAGTGCGTCACCGGCCGCT
GCACCACACCTACCGCCTGACCGTAAACCAGACTTTTTCCGGACAGACCTCAATAACTCTGTTTAC
CAGAACAGGAGGTGAGCTTAGAAAACCCTTAGGGTATTAGGCCAAAGGCGCAGCTACTGTGGGGT
TTATGAACAATTCAAGCAACTCTACGGGCTATTCTAATTCAGGTTTCTCTAGAAATGGACGGAATT
ATTACAGAGCAGCGCCTGCTAGAAAGACGCAGGGCAGCGGCCGAGCAACAGCGCATGAATCAAG
AGCTCCAAGACATGGTTAACTTGCACCAGTGCAAAAGGGGTATCTTTTGTCTGGTAAAGCAGGCCA
AAGTCACCTACGACAGTAATACCACCGGACACCGCCTTAGCTACAAGTTGCCAACCAAGCGTCAG
AAATTGGTGGTCATGGTGGGAGAAAAGCCCATTACCATAACTCAGCACTCGGTAGAAACCGAAGG
CTGCATTCACTCACCTTGTCAAGGACCTGAGGATCTCTGCACCCTTATTAAGACCCTGTGCGGTCTC
AAAGATCTTATTCCCTTTAACTAATAAAAAAAAATAATAAAGCATCACTTACTTAAAATCAGTTAG
CAAATTTCTGTCCAGTTTATTCAGCAGCACCTCCTTGCCCTCCTCCCAGCTCTGGTATTGCAGCTTC
CTCCTGGCTGCAAACTTTCTCCACAATCTAAATGGAATGTCAGTTTCCTCCTGTTCCTGTCCATCCG
CACCCACTATCTTCATGTTGTTGCAGATGAAGCGCGCAAGACCGTCTGAAGATACCTTCAACCCCG
TGTATCCATATGACACGGAAACCGGTCCTCCAACTGTGCCTTTTCTTACTCCTCCCTTTGTATCCCC
CAATGGGTTTCAAGAGAGTCCCCCTGGGGTACTCTCTTTGCGCCTATCCGAACCTCTAGTTACCTCC
AATGGCATGCTTGCGCTCAAAATGGGCAACGGCCTCTCTCTGGACGAGGCCGGCAACCTTACCTCC
CAAAATGTAACCACTGTGAGCCCACCTCTCAAAAAAACCAAGTCAAACATAAACCTGGAAATATC
TGCACCCCTCACAGTTACCTCAGAAGCCCTAACTGTGGCTGCCGCCGCACCTCTAATGGTCGCGGG
CAACACACTCACCATGCAATCACAGGCCCCGCTAACCGTGCACGACTCCAAACTTAGCATTGCCAC
CCAAGGACCCCTCACAGTGTCAGAAGGAAAGCTAGCCCTGCAAACATCAGGCCCCCTCACCACCA
CCGATAGCAGTACCCTTACTATCACTGCCTCACCCCCTCTAACTACTGCCACTGGTAGCTTGGGCAT
TGACTTGAAAGAGCCCATTTATACACAAAATGGAAAACTAGGACTAAAGTACGGGGCTCCTTTGC
ATGTAACAGACGACCTAAACACTTTGACCGTAGCAACTGGTCCAGGTGTGACTATTAATAATACTT
CCTTGCAAACTAAAGTTACTGGAGCCTTGGGTTTTGATTCACAAGGCAATATGCAACTTAATGTAG
CAGGAGGACTAAGGATTGATTCTCAAAACAGACGCCTTATACTTGATGTTAGTTATCCGTTTGATG
CTCAAAACCAACTAAATCTAAGACTAGGACAGGGCCCTCTTTTTATAAACTCAGCCCACAACTTGG
ATATTAACTACAACAAAGGCCTTTACTTGTTTACAGCTTCAAACAATTCCAAAAAGCTTGAGGTTA
ACCTAAGCACTGCCAAGGGGTTGATGTTTGACGCTACAGCCATAGCCATTAATGCAGGAGATGGG
CTTGAATTTGGTTCACCTAATGCACCAAACACAAATCCCCTCAAAACAAAAATTGGCCATGGCCTA
GAATTTGATTCAAACAAGGCTATGGTTCCTAAACTAGGAACTGGCCTTAGTTTTGACAGCACAGGT
GCCATTACAGTAGGAAACAAAAATAATGATAAGCTAACTTTGTGGACCACACCAGCTCCATCTCCT
AACTGTAGACTAAATGCAGAGAAAGATGCTAAACTCACTTTGGTCTTAACAAAATGTGGCAGTCA
AATACTTGCTACAGTTTCAGTTTTGGCTGTTAAAGGCAGTTTGGCTCCAATATCTGGAACAGTTCA
AAGTGCTCATCTTATTATAAGATTTGACGAAAATGGAGTGCTACTAAACAATTCCTTCCTGGACCC
AGAATATTGGAACTTTAGAAATGGAGATCTTACTGAAGGCACAGCCTATACAAACGCTGTTGGATT
TATGCCTAACCTATCAGCTTATCCAAAATCTCACGGTAAAACTGCCAAAAGTAACATTGTCAGTCA
AGTTTACTTAAACGGAGACAAAACTAAACCTGTAACACTAACCATTACACTAAACGGTACACAGG
AAACAGGAGACACAACTCCAAGTGCATACTCTATGTCATTTTCATGGGACTGGTCTGGCCACAACT
ACATTAATGAAATATTTGCCACATCCTCTTACACTTTTTCATACATTGCCCAAGAATAAAGAATCGT
TTGTGTTATGTTTCAACGTGTTTATTTTTCAATTGCAGAAAATTTCGAATCATTTTTCATTCAGTAGT
ATAGCCCCACCACCACATAGCTTATACAGATCACCGTACCTTAATCAAACTCACAGAACCCTAGTA
TTCAACCTGCCACCTCCCTCCCAACACACAGAGTACACAGTCCTTTCTCCCCGGCTGGCCTTAAAA
AGCATCATATCATGGGTAACAGACATATTCTTAGGTGTTATATTCCACACGGTTTCCTGTCGAGCC
AAACGCTCATCAGTGATATTAATAAACTCCCCGGGCAGCTCACTTAAGTTCATGTCGCTGTCCAGC
TGCTGAGCCACAGGCTGCTGTCCAACTTGCGGTTGCTTAACGGGCGGCGAAGGAGAAGTCCACGC
CTACATGGGGGTAGAGTCATAATCGTGCATCAGGATAGGGCGGTGGTGCTGCAGCAGCGCGCGAA
TAAACTGCTGCCGCCGCCGCTCCGTCCTGCAGGAATACAACATGGCAGTGGTCTCCTCAGCGATGA
TTCGCACCGCCCGCAGCATAAGGCGCCTTGTCCTCCGGGCACAGCAGCGCACCCTGATCTCACTTA
AATCAGCACAGTAACTGCAGCACAGCACCACAATATTGTTCAAAATCCCACAGTGCAAGGCGCTG
TATCCAAAGCTCATGGCGGGGACCACAGAACCCACGTGGCCATCATACCACAAGCGCAGGTAGAT
TAAGTGGCGACCCCTCATAAACACGCTGGACATAAACATTACCTCTTTTGGCATGTTGTAATTCAC
CACCTCCCGGTACCATATAAACCTCTGATTAAACATGGCGCCATCCACCACCATCCTAAACCAGCT
GGCCAAAACCTGCCCGCCGGCTATACACTGCAGGGAACCGGGACTGGAACAATGACAGTGGAGA
GCCCAGGACTCGTAACCATGGATCATCATGCTCGTCATGATATCAATGTTGGCACAACACAGGCAC
ACGTGCATACACTTCCTCAGGATTACAAGCTCCTCCCGCGTTAGAACCATATCCCAGGGAACAACC
CATTCCTGAATCAGCGTAAATCCCACACTGCAGGGAAGACCTCGCACGTAACTCACGTTGTGCATT
GTCAAAGTGTTACATTCGGGCAGCAGCGGATGATCCTCCAGTATGGTAGCGCGGGTTTCTGTCTCA
AAAGGAGGTAGACGATCCCTACTGTACGGAGTGCGCCGAGACAACCGAGATCGTGTTGGTCGTAG
TGTCATGCCAAATGGAACGCCGGACGTAGTCATATTTCCTGAAGCAAAACCAGGTGCGGGCGTGA
CAAACAGATCTGCGTCTCCGGTCTCGCCGCTTAGATCGCTCTGTGTAGTAGTTGTAGTATATCCACT
CTCTCAAAGCATCCAGGCGCCCCCTGGCTTCGGGTTCTATGTAAACTCCTTCATGCGCCGCTGCCCT
GATAACATCCACCACCGCAGAATAAGCCACACCCAGCCAACCTACACATTCGTTCTGCGAGTCAC
ACACGGGAGGAGCGGGAAGAGCTGGAAGAACCATGTTTTTTTTTTTATTCCAAAAGATTATCCAAA
ACCTCAAAATGAAGATCTATTAAGTGAACGCGCTCCCCTCCGGTGGCGTGGTCAAACTCTACAGCC
AAAGAACAGATAATGGCATTTGTAAGATGTTGCACAATGGCTTCCAAAAGGCAAACGGCCCTCAC
GTCCAAGTGGACGTAAAGGCTAAACCCTTCAGGGTGAATCTCCTCTATAAACATTCCAGCACCTTC
AACCATGCCCAAATAATTCTCATCTCGCCACCTTCTCAATATATCTCTAAGCAAATCCCGAATATTA
AGTCCGGCCATTGTAAAAATCTGCTCCAGAGCGCCCTCCACCTTCAGCCTCAAGCAGCGAATCATG
ATTGCAAAAATTCAGGTTCCTCACAGACCTGTATAAGATTCAAAAGCGGAACATTAACAAAAATA
CCGCGATCCCGTAGGTCCCTTCGCAGGGCCAGCTGAACATAATCGTGCAGGTCTGCACGGACCAG
CGCGGCCACTTCCCCGCCAGGAACCATGACAAAAGAACCCACACTGATTATGACACGCATACTCG
GAGCTATGCTAACCAGCGTAGCCCCGATGTAAGCTTGTTGCATGGGCGGCGATATAAAATGCAAG
GTGCTGCTCAAAAAATCAGGCAAAGCCTCGCGCAAAAAAGAAAGCACATCGTAGTCATGCTCATG
CAGATAAAGGCAGGTAAGCTCCGGAACCACCACAGAAAAAGACACCATTTTTCTCTCAAACATGT
CTGCGGGTTTCTGCATAAACACAAAATAAAATAACAAAAAAACATTTAAACATTAGAAGCCTGTC
TTACAACAGGAAAAACAACCCTTATAAGCATAAGACGGACTACGGCCATGCCGGCGTGACCGTAA
AAAAACTGGTCACCGTGATTAAAAAGCACCACCGACAGCTCCTCGGTCATGTCCGGAGTCATAAT
GTAAGACTCGGTAAACACATCAGGTTGATTCACATCGGTCAGTGCTAAAAAGCGACCGAAATAGC
CCGGGGGAATACATACCCGCAGGCGTAGAGACAACATTACAGCCCCCATAGGAGGTATAACAAAA
TTAATAGGAGAGAAAAACACATAAACACCTGAAAAACCCTCCTGCCTAGGCAAAATAGCACCCTC
CCGCTCCAGAACAACATACAGCGCTTCCACAGCGGCAGCCATAACAGTCAGCCTTACCAGTAAAA
AAGAAAACCTATTAAAAAAACACCACTCGACACGGCACCAGCTCAATCAGTCACAGTGTAAAAAA
GGGCCAAGTGCAGAGCGAGTATATATAGGACTAAAAAATGACGTAACGGTTAAAGTCCACAAAA
AACACCCAGAAAACCGCACGCGAACCTACGCCCAGAAACGAAAGCCAAAAAACCCACAACTTCCT
CAAATCGTCACTTCCGTTTTCCCACGTTACGTCACTTCCCATTTTAAGAAAACTACAATTCCCAACA
CATACAAGTTACTCCGCCCTAAAACCTACGTCACCCGCCCCGTTCCCACGCCCCGCGCCACGTCAC
AAACTCCACCCCCTCATTATCATATTGGCTTCAATCCAAAATAAGGTATATTATTGATGATGTTAAT
TAATTTAAATCCGCATGCGATATCGAGCTCTCCCGGGAATTCGGATCTGCGACGCGAGGCTGGATG
GCCTTCCCCATTATGATTCTTCTCGCGTTTAAGGGCACCAATAACTGCCTTAAAAAAATTACGCCCC
GCCCTGCCACTCATCGCAGTACTGTTGTAATTCATTAAGCATTCTGCCGACATGGAAGCCATCACA
AACGGCATGATGAACCTGAATCGCCAGCGGCATCAGCACCTTGTCGCCTTGCGTATAATATTTGCC
CATGGTGAAAACGGGGGCGAAGAAGTTGTCCATATTGGCCACGTTTAAATCAAAACTGGTGAAAC
TCACCCAGGGATTGGCTGAGACGAAAAACATATTCTCAATAAACCCTTTAGGGAAATAGGCCAGG
TTTTCACCGTAACACGCCACATCTTGCGAATATATGTGTAGAAACTGCCGGAAATCGTCGTGGTAT
TCACTCCAGAGCGATGAAAACGTTTCAGTTTGCTCATGGAAAACGGTGTAACAAGGGTGAACACT
ATCCCATATCACCAGCTCACCGTCTTTCATTGCCATACGGAATTCCGGATGAGCATTCATCAGGCG
GGCAAGAATGTGAATAAAGGCCGGATAAAACTTGTGCTTATTTTTCTTTACGGTCTTTAAAAAGGC
CGTAATATCCAGCTGAACGGTCTGGTTATAGGTACATTGAGCAACTGACTGAAATGCCTCAAAATG
TTCTTTACGATGCCATTGGGATATATCAACGGTGGTATATCCAGTGATTTTTTTCTCCATTTTAGCTT
CCTTAGCTCCTGAAAATCTCGATAACTCAAAAAATACGCCCGGTAGTGATCTTATTTCATTATGGT
GAAAGTTGGAACCTCTTACGTGCCGATCAACGTCTCATTTTCGCCAAAAGTTGGCCCAGGGCTTCC
CGGTATCAACAGGGACACCAGGATTTATTTATTCTGCGAAGTGATCTTCCGTCACAGGTATTTATT
CGCGATAAGCTCATGGAGCGGCGTAACCGTCGCACAGGAAGGACAGAGAAAGCGCGGATCTGGG
AAGTGACGGACAGAACGGTCAGGACCTGGATTGGGGAGGCGGTTGCCGCCGCTGCTGCTGACGGT
GTGACGTTCTCTGTTCCGGTCACACCACATACGTTCCGCCATTCCTATGCGATGCACATGCTGTATG
CCGGTATACCGCTGAAAGTTCTGCAAAGCCTGATGGGACATAAGTCCATCAGTTCAACGGAAGTCT
ACACGAAGGTTTTTGCGCTGGATGTGGCTGCCCGGCACCGGGTGCAGTTTGCGATGCCGGAGTCTG
ATGCGGTTGCGATGCTGAAACAATTATCCTGAGAATAAATGCCTTGGCCTTTATATGGAAATGTGG
AACTGAGTGGATATGCTGTTTTTGTCTGTTAAACAGAGAAGCTGGCTGTTATCCACTGAGAAGCGA
ACGAAACAGTCGGGAAAATCTCCCATTATCGTAGAGATCCGCATTATTAATCTCAGGAGCCTGTGT
AGCGTTTATAGGAAGTAGTGTTCTGTCATGATGCCTGCAAGCGGTAACGAAAACGATTTGAATATG
CCTTCAGGAACAATAGAAATCTTCGTGCGGTGTTACGTTGAAGTGGAGCGGATTATGTCAGCAATG
GACAGAACAACCTAATGAACACAGAACCATGATGTGGTCTGTCCTTTTACAGCCAGTAGTGCTCGC
CGCAGTCGAGCGACAGGGCGAAGCCCTCGAGTGAGCGAGGAAGCACCAGGGAACAGCACTTATA
TATTCTGCTTACACACGATGCCTGAAAAAACTTCCCTTGGGGTTATCCACTTATCCACGGGGATATT
TTTATAATTATTTTTTTTATAGTTTTTAGATCTTCTTTTTTAGAGCGCCTTGTAGGCCTTTATCCATG
CTGGTTCTAGAGAAGGTGTTGTGACAAATTGCCCTTTCAGTGTGACAAATCACCCTCAAATGACAG
TCCTGTCTGTGACAAATTGCCCTTAACCCTGTGACAAATTGCCCTCAGAAGAAGCTGTTTTTTCACA
AAGTTATCCCTGCTTATTGACTCTTTTTTATTTAGTGTGACAATCTAAAAACTTGTCACACTTCACA
TGGATCTGTCATGGCGGAAACAGCGGTTATCAATCACAAGAAACGTAAAAATAGCCCGCGAATCG
TCCAGTCAAACGACCTCACTGAGGCGGCATATAGTCTCTCCCGGGATCAAAAACGTATGCTGTATC
TGTTCGTTGACCAGATCAGAAAATCTGATGGCACCCTACAGGAACATGACGGTATCTGCGAGATCC
ATGTTGCTAAATATGCTGAAATATTCGGATTGACCTCTGCGGAAGCCAGTAAGGATATACGGCAG
GCATTGAAGAGTTTCGCGGGGAAGGAAGTGGTTTTTTATCGCCCTGAAGAGGATGCCGGCGATGA
AAAAGGCTATGAATCTTTTCCTTGGTTTATCAAACGTGCGCACAGTCCATCCAGAGGGCTTTACAG
TGTACATATCAACCCATATCTCATTCCCTTCTTTATCGGGTTACAGAACCGGTTTACGCAGTTTCGG
CTTAGTGAAACAAAAGAAATCACCAATCCGTATGCCATGCGTTTATACGAATCCCTGTGTCAGTAT
CGTAAGCCGGATGGCTCAGGCATCGTCTCTCTGAAAATCGACTGGATCATAGAGCGTTACCAGCTG
CCTCAAAGTTACCAGCGTATGCCTGACTTCCGCCGCCGCTTCCTGCAGGTCTGTGTTAATGAGATC
AACAGCAGAACTCCAATGCGCCTCTCATACATTGAGAAAAAGAAAGGCCGCCAGACGACTCATAT
CGTATTTTCCTTCCGCGATATCACTTCCATGACGACAGGATAGTCTGAGGGTTATCTGTCACAGATT
TGAGGGTGGTTCGTCACATTTGTTCTGACCTACTGAGGGTAATTTGTCACAGTTTTGCTGTTTCCTT
CAGCCTGCATGGATTTTCTCATACTTTTTGAACTGTAATTTTTAAGGAAGCCAAATTTGAGGGCAGT
TTGTCACAGTTGATTTCCTTCTCTTTCCCTTCGTCATGTGACCTGATATCGGGGGTTAGTTCGTCATC
ATTGATGAGGGTTGATTATCACAGTTTATTACTCTGAATTGGCTATCCGCGTGTGTACCTCTACCTG
GAGTTTTTCCCACGGTGGATATTTCTTCTTGCGCTGAGCGTAAGAGCTATCTGACAGAACAGTTCTT
CTTTGCTTCCTCGCCAGTTCGCTCGCTATGCTCGGTTACACGGCTGCGGCGAGCGCTAGTGATAAT
AAGTGACTGAGGTATGTGCTCTTCTTATCTCCTTTTGTAGTGTTGCTCTTATTTTAAACAACTTTGCG
GTTTTTTGATGACTTTGCGATTTTGTTGTTGCTTTGCAGTAAATTGCAAGATTTAATAAAAAAACGC
AAAGCAATGATTAAAGGATGTTCAGAATGAAACTCATGGAAACACTTAACCAGTGCATAAACGCT
GGTCATGAAATGACGAAGGCTATCGCCATTGCACAGTTTAATGATGACAGCCCGGAAGCGAGGAA
AATAACCCGGCGCTGGAGAATAGGTGAAGCAGCGGATTTAGTTGGGGTTTCTTCTCAGGCTATCAG
AGATGCCGAGAAAGCAGGGCGACTACCGCACCCGGATATGGAAATTCGAGGACGGGTTGAGCAA
CGTGTTGGTTATACAATTGAACAAATTAATCATATGCGTGATGTGTTTGGTACGCGATTGCGACGT
GCTGAAGACGTATTTCCACCGGTGATCGGGGTTGCTGCCCATAAAGGTGGCGTTTACAAAACCTCA
GTTTCTGTTCATCTTGCTCAGGATCTGGCTCTGAAGGGGCTACGTGTTTTGCTCGTGGAAGGTAACG
ACCCCCAGGGAACAGCCTCAATGTATCACGGATGGGTACCAGATCTTCATATTCATGCAGAAGAC
ACTCTCCTGCCTTTCTATCTTGGGGAAAAGGACGATGTCACTTATGCAATAAAGCCCACTTGCTGG
CCGGGGCTTGACATTATTCCTTCCTGTCTGGCTCTGCACCGTATTGAAACTGAGTTAATGGGCAAA
TTTGATGAAGGTAAACTGCCCACCGATCCACACCTGATGCTCCGACTGGCCATTGAAACTGTTGCT
CATGACTATGATGTCATAGTTATTGACAGCGCGCCTAACCTGGGTATCGGCACGATTAATGTCGTA
TGTGCTGCTGATGTGCTGATTGTTCCCACGCCTGCTGAGTTGTTTGACTACACCTCCGCACTGCAGT
TTTTCGATATGCTTCGTGATCTGCTCAAGAACGTTGATCTTAAAGGGTTCGAGCCTGATGTACGTAT
TTTGCTTACCAAATACAGCAATAGTAATGGCTCTCAGTCCCCGTGGATGGAGGAGCAAATTCGGGA
TGCCTGGGGAAGCATGGTTCTAAAAAATGTTGTACGTGAAACGGATGAAGTTGGTAAAGGTCAGA
TCCGGATGAGAACTGTTTTTGAACAGGCCATTGATCAACGCTCTTCAACTGGTGCCTGGAGAAATG
CTCTTTCTATTTGGGAACCTGTCTGCAATGAAATTTTCGATCGTCTGATTAAACCACGCTGGGAGAT
TAGATAATGAAGCGTGCGCCTGTTATTCCAAAACATACGCTCAATACTCAACCGGTTGAAGATACT
TCGTTATCGACACCAGCTGCCCCGATGGTGGATTCGTTAATTGCGCGCGTAGGAGTAATGGCTCGC
GGTAATGCCATTACTTTGCCTGTATGTGGTCGGGATGTGAAGTTTACTCTTGAAGTGCTCCGGGGT
GATAGTGTTGAGAAGACCTCTCGGGTATGGTCAGGTAATGAACGTGACCAGGAGCTGCTTACTGA
GGACGCACTGGATGATCTCATCCCTTCTTTTCTACTGACTGGTCAACAGACACCGGCGTTCGGTCG
AAGAGTATCTGGTGTCATAGAAATTGCCGATGGGAGTCGCCGTCGTAAAGCTGCTGCACTTACCGA
AAGTGATTATCGTGTTCTGGTTGGCGAGCTGGATGATGAGCAGATGGCTGCATTATCCAGATTGGG
TAACGATTATCGCCCAACAAGTGCTTATGAACGTGGTCAGCGTTATGCAAGCCGATTGCAGAATGA
ATTTGCTGGAAATATTTCTGCGCTGGCTGATGCGGAAAATATTTCACGTAAGATTATTACCCGCTG
TATCAACACCGCCAAATTGCCTAAATCAGTTGTTGCTCTTTTTTCTCACCCCGGTGAACTATCTGCC
CGGTCAGGTGATGCACTTCAAAAAGCCTTTACAGATAAAGAGGAATTACTTAAGCAGCAGGCATC
TAACCTTCATGAGCAGAAAAAAGCTGGGGTGATATTTGAAGCTGAAGAAGTTATCACTCTTTTAAC
TTCTGTGCTTAAAACGTCATCTGCATCAAGAACTAGTTTAAGCTCACGACATCAGTTTGCTCCTGG
AGCGACAGTATTGTATAAGGGCGATAAAATGGTGCTTAACCTGGACAGGTCTCGTGTTCCAACTGA
GTGTATAGAGAAAATTGAGGCCATTCTTAAGGAACTTGAAAAGCCAGCACCCTGATGCGACCACG
TTTTAGTCTACGTTTATCTGTCTTTACTTAATGTCCTTTGTTACAGGCCAGAAAGCATAACTGGCCT
GAATATTCTCTCTGGGCCCACTGTTCCACTTGTATCGTCGGTCTGATAATCAGACTGGGACCACGG
TCCCACTCGTATCGTCGGTCTGATTATTAGTCTGGGACCACGGTCCCACTCGTATCGTCGGTCTGAT
TATTAGTCTGGGACCACGGTCCCACTCGTATCGTCGGTCTGATAATCAGACTGGGACCACGGTCCC
ACTCGTATCGTCGGTCTGATTATTAGTCTGGGACCATGGTCCCACTCGTATCGTCGGTCTGATTATT
AGTCTGGGACCACGGTCCCACTCGTATCGTCGGTCTGATTATTAGTCTGGAACCACGGTCCCACTC
GTATCGTCGGTCTGATTATTAGTCTGGGACCACGGTCCCACTCGTATCGTCGGTCTGATTATTAGTC
TGGGACCACGATCCCACTCGTGTTGTCGGTCTGATTATCGGTCTGGGACCACGGTCCCACTTGTATT
GTCGATCAGACTATCAGCGTGAGACTACGATTCCATCAATGCCTGTCAAGGGCAAGTATTGACATG
TCGTCGTAACCTGTAGAACGGAGTAACCTCGGTGTGCGGTTGTATGCCTGCTGTGGATTGCTGCTG
TGTCCTGCTTATCCACAACATTTTGCGCACGGTTATGTGGACAAAATACCTGGTTACCCAGGCCGT
GCCGGCACGTTAACCGGGCACATTTCCCCGAAAAGTGCCACCTGACGTCTAAGAAACCATTATTAT
CATGACATTAACCTATAAAAATAGGCGTATCACGAGGCCCTTTCGTCTTCAAGAATTGGATCCGAA
TTCCCGGGAGAGCTCGATATCGCATGCGGATTTAAATTAATTAA
* C1D
(SEQ ID NO: 85)
CATCATCAATAATATACCTTATTTTGGATTGAAGCCAATATGATAATGAGGGGGTGGAGTTTGTGA
CGTGGCGCGGGGCGTGGGAACGGGGCGGGTGACGTAGTAGTGTGGCGGAAGTGTGATGTTGCAAG
TGTGGCGGAACACATGTAAGCGACGGATGTGGCAAAAGTGACGTTTTTGGTGTGCGCCGGTGTAC
ACAGGAAGTGACAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAATTTTGTGTTACT
CATAGCGCGTAATATTTGTCTAGGGCCGCGGGGACTTTGACCGTTTACGTGGAGACTCGCCCAGGT
GTTTTTCTCAGGTGTTTTCCGCGTTCCGGGTCAAAGTTGGCGTTTTATTATTATAGTCAGTCGAAGC
TTGGATCCGGTACCTCTAGAATTCTCGAGCGGCCGCTAGCGACATCGGATCTCCCGATCCCCTATG
GTGCACTCTCAGTACAATCTGCTCTGATGCCGCATAGTTAAGCCAGTATCTGCTCCCTGCTTGTGTG
TTGGAGGTCGCTGAGTAGTGCGCGAGCAAAATTTAAGCTACAACAAGGCAAGGCTTGACCGACAA
TTGCATGAAGAATCTGCTTAGGGTTAGGCGTTTTGCGCTGCTTCGCGATGTACGGGCCAGATATAC
GCGTTGACATTGATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACG
TCAATGGGTGGACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCTCTG
GCTAACTAGAGAACCCACTGCTTACTGGCTTATCGAAATTAATACGACTCACTATAGGGAGACCCA
AGCTGGCTAGTTAAGCTATCAACAAGTTTGTACAAAAAAGCAGGCTTTAAAGGAACCAATTCAGT
CGACTGGATCCGGTACCACCATGTTCCTGAACTGCTGCCCAGGTTGCTGTATGGAGCCTGAATTCA
CCATGGTGAGCAAGGGCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACCCT
GACCTGGGGCGTGCAGTGCTTCGCCCGCTACCCCGACCACATGAAGCAGCACGACTTCTTCAAGTC
CGCCATGCCCGAAGGCTACGTCCAGGAGCGCACCATCTTCTTCAAGGACGACGGCAACTACAAGA
CCCGCGCCGAGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACGCCATCAGCGACAACGTCTA
TATCACCGCCGACAAGCAGAAGAACGGCATCAAGGCCAACTTCAAGATCCGCCACAACATCGAGG
ACGGCAGCGTGCAGCTCGCCGACCACTACCAGCAGAACACCCCCATCGGCGACGGCCCCGTGCTG
CTGCCCGACAACCACTACCTGAGCACCCAGTCCAAGCTGAGCAAAGACCCCAACGAGAAGCGCGA
TCACATGGTCCTGCTGGAGTTCGTGACCGCCGCCGGGATCACTCTCGGCATGGACGAGCTGTACAA
GGTCGACTATCCGTACGACGTACCAGACTACGCATAACCGCGGCCGCACTCGAGATATCTAGACC
CAGCTTTCTTGTACAAAGTGGTTGATCTAGAGGGCCCGCGGTTCGAAGGTAAGCCTATCCCTAACC
CTCTCCTCGGTCTCGATTCTACGCGTACCGGTTAGTAATGAGTTTAAACGGGGGAGGCTAACTGAA
ACACGGAAGGAGACAATACCGGAAGGAACCCGCGCTATGACGGCAATAAAAAGACAGAATAAAA
CGCACGGGTGTTGGGTCGTTTGTTCATAAACGCGGGGTTCGGTCCCAGGGCTGGCACTCTGTCGAT
ACCCCACCGAGACCCCATTGGGGCCAATACGCCCGCGTTTCTTCCTTTTCCCCACCCCACCCCCCA
AGTTCGGGTGAAGGCCCAGGGCTCGCAGCCAACGTCGGGGCGGCAGGCCCTGCCATAGCAGATCC
GATTCGACAGATCACTGAAATGTGTGGGCGTGGCTTAAGGGTGGGAAAGAATATATAAGGTGGGG
GTCTTATGTAGTTTTGTATCTGTTTTGCAGCAGCCGCCGCCGCCATGAGCACCAACTCGTTTGATGG
AAGCATTGTGAGCTCATATTTGACAACGCGCATGCCCCCATGGGCCGGGGTGCGTCAGAATGTGAT
GGGCTCCAGCATTGATGGTCGCCCCGTCCTGCCCGCAAACTCTACTACCTTGACCTACGAGACCGT
GTCTGGAACGCCGTTGGAGACTGCAGCCTCCGCCGCCGCTTCAGCCGCTGCAGCCACCGCCCGCGG
GATTGTGACTGACTTTGCTTTCCTGAGCCCGCTTGCAAGCAGTGCAGCTTCCCGTTCATCCGCCCGC
GATGACAAGTTGACGGCTCTTTTGGCACAATTGGATTCTTTGACCCGGGAACTTAATGTCGTTTCTC
AGCAGCTGTTGGATCTGCGCCAGCAGGTTTCTGCCCTGAAGGCTTCCTCCCCTCCCAATGCGGTTT
AAAACATAAATAAAAAACCAGACTCTGTTTGGATTTGGATCAAGCAAGTGTCTTGCTGTCTTTATT
TAGGGGTTTTGCGCGCGCGGTAGGCCCGGGACCAGCGGTCTCGGTCGTTGAGGGTCCTGTGTATTT
TTTCCAGGACGTGGTAAAGGTGACTCTGGATGTTCAGATACATGGGCATAAGCCCGTCTCTGGGGT
GGAGGTAGCACCACTGCAGAGCTTCATGCTGCGGGGTGGTGTTGTAGATGATCCAGTCGTAGCAG
GAGCGCTGGGCGTGGTGCCTAAAAATGTCTTTCAGTAGCAAGCTGATTGCCAGGGGCAGGCCCTT
GGTGTAAGTGTTTACAAAGCGGTTAAGCTGGGATGGGTGCATACGTGGGGATATGAGATGCATCT
TGGACTGTATTTTTAGGTTGGCTATGTTCCCAGCCATATCCCTCCGGGGATTCATGTTGTGCAGAAC
CACCAGCACAGTGTATCCGGTGCACTTGGGAAATTTGTCATGTAGCTTAGAAGGAAATGCGTGGA
AGAACTTGGAGACGCCCTTGTGACCTCCAAGATTTTCCATGCATTCGTCCATAATGATGGCAATGG
GCCCACGGGCGGCGGCCTGGGCGAAGATATTTCTGGGATCACTAACGTCATAGTTGTGTTCCAGGA
TGAGATCGTCATAGGCCATTTTTACAAAGCGCGGGCGGAGGGTGCCAGACTGCGGTATAATGGTT
CCATCCGGCCCAGGGGCGTAGTTACCCTCACAGATTTGCATTTCCCACGCTTTGAGTTCAGATGGG
GGGATCATGTCTACCTGCGGGGCGATGAAGAAAACGGTTTCCGGGGTAGGGGAGATCAGCTGGGA
AGAAAGCAGGTTCCTGAGCAGCTGCGACTTACCGCAGCCGGTGGGCCCGTAAATCACACCTATTA
CCGGCTGCAACTGGTAGTTAAGAGAGCTGCAGCTGCCGTCATCCCTGAGCAGGGGGGCCACTTCG
TTAAGCATGTCCCTGACTCGCATGTTTTCCCTGACCAAATCCGCCAGAAGGCGCTCGCCGCCCAGC
GATAGCAGTTCTTGCAAGGAAGCAAAGTTTTTCAACGGTTTGAGACCGTCCGCCGTAGGCATGCTT
TTGAGCGTTTGACCAAGCAGTTCCAGGCGGTCCCACAGCTCGGTCACCTGCTCTACGGCATCTCGA
TCCAGCATATCTCCTCGTTTCGCGGGTTGGGGCGGCTTTCGCTGTACGGCAGTAGTCGGTGCTCGTC
CAGACGGGCCAGGGTCATGTCTTTCCACGGGCGCAGGGTCCTCGTCAGCGTAGTCTGGGTCACGGT
GAAGGGGTGCGCTCCGGGCTGCGCGCTGGCCAGGGTGCGCTTGAGGCTGGTCCTGCTGGTGCTGA
AGCGCTGCCGGTCTTCGCCCTGCGCGTCGGCCAGGTAGCATTTGACCATGGTGTCATAGTCCAGCC
CCTCCGCGGCGTGGCCCTTGGCGCGCAGCTTGCCCTTGGAGGAGGCGCCGCACGAGGGGCAGTGC
AGACTTTTGAGGGCGTAGAGCTTGGGCGCGAGAAATACCGATTCCGGGGAGTAGGCATCCGCGCC
GCAGGCCCCGCAGACGGTCTCGCATTCCACGAGCCAGGTGAGCTCTGGCCGTTCGGGGTCAAAAA
CCAGGTTTCCCCCATGCTTTTTGATGCGTTTCTTACCTCTGGTTTCCATGAGCCGGTGTCCACGCTC
GGTGACGAAAAGGCTGTCCGTGTCCCCGTATACAGACTTGAGAGGCCTGTCCTCGAGCGGTGTTCC
GCGGTCCTCCTCGTATAGAAACTCGGACCACTCTGAGACAAAGGCTCGCGTCCAGGCCAGCACGA
AGGAGGCTAAGTGGGAGGGGTAGCGGTCGTTGTCCACTAGGGGGTCCACTCGCTCCAGGGTGTGA
AGACACATGTCGCCCTCTTCGGCATCAAGGAAGGTGATTGGTTTGTAGGTGTAGGCCACGTGACCG
GGTGTTCCTGAAGGGGGGCTATAAAAGGGGGTGGGGGCGCGTTCGTCCTCACTCTCTTCCGCATCG
CTGTCTGCGAGGGCCAGCTGTTGGGGTGAGTACTCCCTCTGAAAAGCGGGCATGACTTCTGCGCTA
AGATTGTCAGTTTCCAAAAACGAGGAGGATTTGATATTCACCTGGCCCGCGGTGATGCCTTTGAGG
GTGGCCGCATCCATCTGGTCAGAAAAGACAATCTTTTTGTTGTCAAGCTTGGTGGCAAACGACCCG
TAGAGGGCGTTGGACAGCAACTTGGCGATGGAGCGCAGGGTTTGGTTTTTGTCGCGATCGGCGCG
CTCCTTGGCCGCGATGTTTAGCTGCACGTATTCGCGCGCAACGCACCGCCATTCGGGAAAGACGGT
GGTGCGCTCGTCGGGCACCAGGTGCACGCGCCAACCGCGGTTGTGCAGGGTGACAAGGTCAACGC
TGGTGGCTACCTCTCCGCGTAGGCGCTCGTTGGTCCAGCAGAGGCGGCCGCCCTTGCGCGAGCAGA
ATGGCGGTAGGGGGTCTAGCTGCGTCTCGTCCGGGGGGTCTGCGTCCACGGTAAAGACCCCGGGC
AGCAGGCGCGCGTCGAAGTAGTCTATCTTGCATCCTTGCAAGTCTAGCGCCTGCTGCCATGCGCGG
GCGGCAAGCGCGCGCTCGTATGGGTTGAGTGGGGGACCCCATGGCATGGGGTGGGTGAGCGCGGA
GGCGTACATGCCGCAAATGTCGTAAACGTAGAGGGGCTCTCTGAGTATTCCAAGATATGTAGGGT
AGCATCTTCCACCGCGGATGCTGGCGCGCACGTAATCGTATAGTTCGTGCGAGGGAGCGAGGAGG
TCGGGACCGAGGTTGCTACGGGCGGGCTGCTCTGCTCGGAAGACTATCTGCCTGAAGATGGCATGT
GAGTTGGATGATATGGTTGGACGCTGGAAGACGTTGAAGCTGGCGTCTGTGAGACCTACCGCGTC
ACGCACGAAGGAGGCGTAGGAGTCGCGCAGCTTGTTGACCAGCTCGGCGGTGACCTGCACGTCTA
GGGCGCAGTAGTCCAGGGTTTCCTTGATGATGTCATACTTATCCTGTCCCTTTTTTTTCCACAGCTC
GCGGTTGAGGACAAACTCTTCGCGGTCTTTCCAGTACTCTTGGATCGGAAACCCGTCGGCCTCCGA
ACGGTAAGAGCCTAGCATGTAGAACTGGTTGACGGCCTGGTAGGCGCAGCATCCCTTTTCTACGGG
TAGCGCGTATGCCTGCGCGGCCTTCCGGAGCGAGGTGTGGGTGAGCGCAAAGGTGTCCCTGACCA
TGACCAGCATGAAGGGCACGAGCTGCTTCCCAAAGGCCCCCATCCAAGTATAGGTCTCTACATCGT
AGGTGACAAAGAGACGCTCGGTGCGAGGATGCGAGCCGATCGGGAAGAACTGGATCTCCCGCCAC
CAATTGGAGGAGTGGCTATTGATGTGGTGAAAGTAGAAGTCCCTGCGACGGGCCGAACACTCGTG
CTGGCTTTTGTAAAAACGTGCGCAGTACTGGCAGCGGTGCACGGGCTGTACATCCTGCACGAGGTT
GACCTGACGACCGCGCACAAGGAAGCAGAGTGGGAATTTGAGCCCCTCGCCTGGCGGGTTTGGCT
GGTGGTCTTCTACTTCGGCTGCTTGTCCTTGACCGTCTGGCTGCTCGAGGGGAGTTACGGTGGATC
GGACCACCACGCCGCGCGAGCCCAAAGTCCAGATGTCCGCGCGCGGCGGTCGGAGCTTGATGACA
ACATCGCGCAGATGGGAGCTGTCCATGGTCTGGAGCTCCCGCGGCGTCAGGTCAGGCGGGAGCTC
CTGCAGGTTTACCTCGCATAGACGGGTCAGGGCGCGGGCTAGATCCAGGTGATACCTAATTTCCAG
GGGCTGGTTGGTGGCGGCGTCGATGGCTTGCAAGAGGCCGCATCCCCGCGGCGCGACTACGGTAC
CGCGCGGCGGGCGGTGGGCCGCGGGGGTGTCCTTGGATGATGCATCTAAAAGCGGTGACGCGGGC
GAGCCCCCGGAGGTAGGGGGGGCTCCGGACCCGCCGGGAGAGGGGGCAGGGGCACGTCGGCGCC
GCGCGCGGGCAGGAGCTGGTGCTGCGCGCGTAGGTTGCTGGCGAACGCGACGACGCGGCGGTTGA
TCTCCTGAATCTGGCGCCTCTGCGTGAAGACGACGGGCCCGGTGAGCTTGAACCTGAAAGAGAGT
TCGACAGAATCAATTTCGGTGTCGTTGACGGCGGCCTGGCGCAAAATCTCCTGCACGTCTCCTGAG
TTGTCTTGATAGGCGATCTCGGCCATGAACTGCTCGATCTCTTCCTCCTGGAGATCTCCGCGTCCGG
CTCGCTCCACGGTGGCGGCGAGGTCGTTGGAAATGCGGGCCATGAGCTGCGAGAAGGCGTTGAGG
CCTCCCTCGTTCCAGACGCGGCTGTAGACCACGCCCCCTTCGGCATCGCGGGCGCGCATGACCACC
TGCGCGAGATTGAGCTCCACGTGCCGGGCGAAGACGGCGTAGTTTCGCAGGCGCTGAAAGAGGTA
GTTGAGGGTGGTGGCGGTGTGTTCTGCCACGAAGAAGTACATAACCCAGCGTCGCAACGTGGATT
CGTTGATATCCCCCAAGGCCTCAAGGCGCTCCATGGCCTCGTAGAAGTCCACGGCGAAGTTGAAA
AACTGGGAGTTGCGCGCCGACACGGTTAACTCCTCCTCCAGAAGACGGATGAGCTCGGCGACAGT
GTCGCGCACCTCGCGCTCAAAGGCTACAGGGGCCTCTTCTTCTTCTTCAATCTCCTCTTCCATAAGG
GCCTCCCCTTCTTCTTCTTCTGGCGGCGGTGGGGGAGGGGGGACACGGCGGCGACGACGGCGCAC
CGGGAGGCGGTCGACAAAGCGCTCGATCATCTCCCCGCGGCGACGGCGCATGGTCTCGGTGACGG
CGCGGCCGTTCTCGCGGGGGCGCAGTTGGAAGACGCCGCCCGTCATGTCCCGGTTATGGGTTGGCG
GGGGGCTGCCATGCGGCAGGGATACGGCGCTAACGATGCATCTCAACAATTGTTGTGTAGGTACT
CCGCCGCCGAGGGACCTGAGCGAGTCCGCATCGACCGGATCGGAAAACCTCTCGAGAAAGGCGTC
TAACCAGTCACAGTCGCAAGGTAGGCTGAGCACCGTGGCGGGCGGCAGCGGGCGGCGGTCGGGGT
TGTTTCTGGCGGAGGTGCTGCTGATGATGTAATTAAAGTAGGCGGTCTTGAGACGGCGGATGGTCG
ACAGAAGCACCATGTCCTTGGGTCCGGCCTGCTGAATGCGCAGGCGGTCGGCCATGCCCCAGGCTT
CGTTTTGACATCGGCGCAGGTCTTTGTAGTAGTCTTGCATGAGCCTTTCTACCGGCACTTCTTCTTC
TCCTTCCTCTTGTCCTGCATCTCTTGCATCTATCGCTGCGGCGGCGGCGGAGTTTGGCCGTAGGTGG
CGCCCTCTTCCTCCCATGCGTGTGACCCCGAAGCCCCTCATCGGCTGAAGCAGGGCTAGGTCGGCG
ACAACGCGCTCGGCTAATATGGCCTGCTGCACCTGCGTGAGGGTAGACTGGAAGTCATCCATGTCC
ACAAAGCGGTGGTATGCGCCCGTGTTGATGGTGTAAGTGCAGTTGGCCATAACGGACCAGTTAAC
GGTCTGGTGACCCGGCTGCGAGAGCTCGGTGTACCTGAGACGCGAGTAAGCCCTCGAGTCAAATA
CGTAGTCGTTGCAAGTCCGCACCAGGTACTGGTATCCCACCAAAAAGTGCGGCGGCGGCTGGCGG
TAGAGGGGCCAGCGTAGGGTGGCCGGGGCTCCGGGGGCGAGATCTTCCAACATAAGGCGATGATA
TCCGTAGATGTACCTGGACATCCAGGTGATGCCGGCGGCGGTGGTGGAGGCGCGCGGAAAGTCGC
GGACGCGGTTCCAGATGTTGCGCAGCGGCAAAAAGTGCTCCATGGTCGGGACGCTCTGGCCGGTC
AGGCGCGCGCAATCGTTGACGCTCTAGACCGTGCAAAAGGAGAGCCTGTAAGCGGGCACTCTTCC
GTGGTCTGGTGGATAAATTCGCAAGGGTATCATGGCGGACGACCGGGGTTCGAGCCCCGTATCCG
GCCGTCCGCCGTGATCCATGCGGTTACCGCCCGCGTGTCGAACCCAGGTGTGCGACGTCAGACAAC
GGGGGAGTGCTCCTTTTGGCTTCCTTCCAGGCGCGGCGGCTGCTGCGCTAGCTTTTTTGGCCACTGG
CCGCGCGCAGCGTAAGCGGTTAGGCTGGAAAGCGAAAGCATTAAGTGGCTCGCTCCCTGTAGCCG
GAGGGTTATTTTCCAAGGGTTGAGTCGCGGGACCCCCGGTTCGAGTCTCGGACCGGCCGGACTGCG
GCGAACGGGGGTTTGCCTCCCCGTCATGCAAGACCCCGCTTGCAAATTCCTCCGGAAACAGGGAC
GAGCCCCTTTTTTGCTTTTCCCAGATGCATCCGGTGCTGCGGCAGATGCGCCCCCCTCCTCAGCAGC
GGCAAGAGCAAGAGCAGCGGCAGACATGCAGGGCACCCTCCCCTCCTCCTACCGCGTCAGGAGGG
GCGACATCCGCGGTTGACGCGGCAGCAGATGGTGATTACGAACCCCCGCGGCGCCGGGCCCGGCA
CTACCTGGACTTGGAGGAGGGCGAGGGCCTGGCGCGGCTAGGAGCGCCCTCTCCTGAGCGGCACC
CAAGGGTGCAGCTGAAGCGTGATACGCGTGAGGCGTACGTGCCGCGGCAGAACCTGTTTCGCGAC
CGCGAGGGAGAGGAGCCCGAGGAGATGCGGGATCGAAAGTTCCACGCAGGGCGCGAGCTGCGGC
ATGGCCTGAATCGCGAGCGGTTGCTGCGCGAGGAGGACTTTGAGCCCGACGCGCGAACCGGGATT
AGTCCCGCGCGCGCACACGTGGCGGCCGCCGACCTGGTAACCGCATACGAGCAGACGGTGAACCA
GGAGATTAACTTTCAAAAAAGCTTTAACAACCACGTGCGTACGCTTGTGGCGCGCGAGGAGGTGG
CTATAGGACTGATGCATCTGTGGGACTTTGTAAGCGCGCTGGAGCAAAACCCAAATAGCAAGCCG
CTCATGGCGCAGCTGTTCCTTATAGTGCAGCACAGCAGGGACAACGAGGCATTCAGGGATGCGCT
GCTAAACATAGTAGAGCCCGAGGGCCGCTGGCTGCTCGATTTGATAAACATCCTGCAGAGCATAG
TGGTGCAGGAGCGCAGCTTGAGCCTGGCTGACAAGGTGGCCGCCATCAACTATTCCATGCTTAGCC
TGGGCAAGTTTTACGCCCGCAAGATATACCATACCCCTTACGTTCCCATAGACAAGGAGGTAAAG
ATCGAGGGGTTCTACATGCGCATGGCGCTGAAGGTGCTTACCTTGAGCGACGACCTGGGCGTTTAT
CGCAACGAGCGCATCCACAAGGCCGTGAGCGTGAGCCGGCGGCGCGAGCTCAGCGACCGCGAGCT
GATGCACAGCCTGCAAAGGGCCCTGGCTGGCACGGGCAGCGGCGATAGAGAGGCCGAGTCCTACT
TTGACGCGGGCGCTGACCTGCGCTGGGCCCCAAGCCGACGCGCCCTGGAGGCAGCTGGGGCCGGA
CCTGGGCTGGCGGTGGCACCCGCGCGCGCTGGCAACGTCGGCGGCGTGGAGGAATATGACGAGGA
CGATGAGTACGAGCCAGAGGACGGCGAGTACTAAGCGGTGATGTTTCTGATCAGATGATGCAAGA
CGCAACGGACCCGGCGGTGCGGGCGGCGCTGCAGAGCCAGCCGTCCGGCCTTAACTCCACGGACG
ACTGGCGCCAGGTCATGGACCGCATCATGTCGCTGACTGCGCGCAATCCTGACGCGTTCCGGCAGC
AGCCGCAGGCCAACCGGCTCTCCGCAATTCTGGAAGCGGTGGTCCCGGCGCGCGCAAACCCCACG
CACGAGAAGGTGCTGGCGATCGTAAACGCGCTGGCCGAAAACAGGGCCATCCGGCCCGACGAGG
CCGGCCTGGTCTACGACGCGCTGCTTCAGCGCGTGGCTCGTTACAACAGCGGCAACGTGCAGACC
AACCTGGACCGGCTGGTGGGGGATGTGCGCGAGGCCGTGGCGCAGCGTGAGCGCGCGCAGCAGC
AGGGCAACCTGGGCTCCATGGTTGCACTAAACGCCTTCCTGAGTACACAGCCCGCCAACGTGCCGC
GGGGACAGGAGGACTACACCAACTTTGTGAGCGCACTGCGGCTAATGGTGACTGAGACACCGCAA
AGTGAGGTGTACCAGTCTGGGCCAGACTATTTTTTCCAGACCAGTAGACAAGGCCTGCAGACCGTA
AACCTGAGCCAGGCTTTCAAAAACTTGCAGGGGCTGTGGGGGGTGCGGGCTCCCACAGGCGACCG
CGCGACCGTGTCTAGCTTGCTGACGCCCAACTCGCGCCTGTTGCTGCTGCTAATAGCGCCCTTCAC
GGACAGTGGCAGCGTGTCCCGGGACACATACCTAGGTCACTTGCTGACACTGTACCGCGAGGCCA
TAGGTCAGGCGCATGTGGACGAGCATACTTTCCAGGAGATTACAAGTGTCAGCCGCGCGCTGGGG
CAGGAGGACACGGGCAGCCTGGAGGCAACCCTAAACTACCTGCTGACCAACCGGCGGCAGAAGA
TCCCCTCGTTGCACAGTTTAAACAGCGAGGAGGAGCGCATTTTGCGCTACGTGCAGCAGAGCGTG
AGCCTTAACCTGATGCGCGACGGGGTAACGCCCAGCGTGGCGCTGGACATGACCGCGCGCAACAT
GGAACCGGGCATGTATGCCTCAAACCGGCCGTTTATCAACCGCCTAATGGACTACTTGCATCGCGC
GGCCGCCGTGAACCCCGAGTATTTCACCAATGCCATCTTGAACCCGCACTGGCTACCGCCCCCTGG
TTTCTACACCGGGGGATTCGAGGTGCCCGAGGGTAACGATGGATTCCTCTGGGACGACATAGACG
ACAGCGTGTTTTCCCCGCAACCGCAGACCCTGCTAGAGTTGCAACAGCGCGAGCAGGCAGAGGCG
GCGCTGCGAAAGGAAAGCTTCCGCAGGCCAAGCAGCTTGTCCGATCTAGGCGCTGCGGCCCCGCG
GTCAGATGCTAGTAGCCCATTTCCAAGCTTGATAGGGTCTCTTACCAGCACTCGCACCACCCGCCC
GCGCCTGCTGGGCGAGGAGGAGTACCTAAACAACTCGCTGCTGCAGCCGCAGCGCGAAAAAAACC
TGCCTCCGGCATTTCCCAACAACGGGATAGAGAGCCTAGTGGACAAGATGAGTAGATGGAAGACG
TACGCGCAGGAGCACAGGGACGTGCCAGGCCCGCGCCCGCCCACCCGTCGTCAAAGGCACGACCG
TCAGCGGGGTCTGGTGTGGGAGGACGATGACTCGGCAGACGACAGCAGCGTCCTGGATTTGGGAG
GGAGTGGCAACCCGTTTGCGCACCTTCGCCCCAGGCTGGGGAGAATGTTTTAAAAAAAAAAAAAG
CATGATGCAAAATAAAAAACTCACCAAGGCCATGGCACCGAGCGTTGGTTTTCTTGTATTCCCCTT
AGTATGCGGCGCGCGGCGATGTATGAGGAAGGTCCTCCTCCCTCCTACGAGAGTGTGGTGAGCGC
GGCGCCAGTGGCGGCGGCGCTGGGTTCTCCCTTCGATGCTCCCCTGGACCCGCCGTTTGTGCCTCC
GCGGTACCTGCGGCCTACCGGGGGGAGAAACAGCATCCGTTACTCTGAGTTGGCACCCCTATTCGA
CACCACCCGTGTGTACCTGGTGGACAACAAGTCAACGGATGTGGCATCCCTGAACTACCAGAACG
ACCACAGCAACTTTCTGACCACGGTCATTCAAAACAATGACTACAGCCCGGGGGAGGCAAGCACA
CAGACCATCAATCTTGACGACCGGTCGCACTGGGGCGGCGACCTGAAAACCATCCTGCATACCAA
CATGCCAAATGTGAACGAGTTCATGTTTACCAATAAGTTTAAGGCGCGGGTGATGGTGTCGCGCTT
GCCTACTAAGGACAATCAGGTGGAGCTGAAATACGAGTGGGTGGAGTTCACGCTGCCCGAGGGCA
ACTACTCCGAGACCATGACCATAGACCTTATGAACAACGCGATCGTGGAGCACTACTTGAAAGTG
GGCAGACAGAACGGGGTTCTGGAAAGCGACATCGGGGTAAAGTTTGACACCCGCAACTTCAGACT
GGGGTTTGACCCCGTCACTGGTCTTGTCATGCCTGGGGTATATACAAACGAAGCCTTCCATCCAGA
CATCATTTTGCTGCCAGGATGCGGGGTGGACTTCACCCACAGCCGCCTGAGCAACTTGTTGGGCAT
CCGCAAGCGGCAACCCTTCCAGGAGGGCTTTAGGATCACCTACGATGATCTGGAGGGTGGTAACA
TTCCCGCACTGTTGGATGTGGACGCCTACCAGGCGAGCTTGAAAGATGACACCGAACAGGGCGGG
GGTGGCGCAGGCGGCAGCAACAGCAGTGGCAGCGGCGCGGAAGAGAACTCCAACGCGGCAGCCG
CGGCAATGCAGCCGGTGGAGGACATGAACGATCATGCCATTCGCGGCGACACCTTTGCCACACGG
GCTGAGGAGAAGCGCGCTGAGGCCGAAGCAGCGGCCGAAGCTGCCGCCCCCGCTGCGCAACCCG
AGGTCGAGAAGCCTCAGAAGAAACCGGTGATCAAACCCCTGACAGAGGACAGCAAGAAACGCAG
TTACAACCTAATAAGCAATGACAGCACCTTCACCCAGTACCGCAGCTGGTACCTTGCATACAACTA
CGGCGACCCTCAGACCGGAATCCGCTCATGGACCCTGCTTTGCACTCCTGACGTAACCTGCGGCTC
GGAGCAGGTCTACTGGTCGTTGCCAGACATGATGCAAGACCCCGTGACCTTCCGCTCCACGCGCCA
GATCAGCAACTTTCCGGTGGTGGGCGCCGAGCTGTTGCCCGTGCACTCCAAGAGCTTCTACAACGA
CCAGGCCGTCTACTCCCAACTCATCCGCCAGTTTACCTCTCTGACCCACGTGTTCAATCGCTTTCCC
GAGAACCAGATTTTGGCGCGCCCGCCAGCCCCCACCATCACCACCGTCAGTGAAAACGTTCCTGCT
CTCACAGATCACGGGACGCTACCGCTGCGCAACAGCATCGGAGGAGTCCAGCGAGTGACCATTAC
TGACGCCAGACGCCGCACCTGCCCCTACGTTTACAAGGCCCTGGGCATAGTCTCGCCGCGCGTCCT
ATCGAGCCGCACTTTTTGAGCAAGCATGTCCATCCTTATATCGCCCAGCAATAACACAGGCTGGGG
CCTGCGCTTCCCAAGCAAGATGTTTGGCGGGGCCAAGAAGCGCTCCGACCAACACCCAGTGCGCG
TGCGCGGGCACTACCGCGCGCCCTGGGGCGCGCACAAACGCGGCCGCACTGGGCGCACCACCGTC
GATGACGCCATCGACGCGGTGGTGGAGGAGGCGCGCAACTACACGCCCACGCCGCCACCAGTGTC
CACAGTGGACGCGGCCATTCAGACCGTGGTGCGCGGAGCCCGGCGCTATGCTAAAATGAAGAGAC
GGCGGAGGCGCGTAGCACGTCGCCACCGCCGCCGACCCGGCACTGCCGCCCAACGCGCGGCGGCG
GCCCTGCTTAACCGCGCACGTCGCACCGGCCGACGGGCGGCCATGCGGGCCGCTCGAAGGCTGGC
CGCGGGTATTGTCACTGTGCCCCCCAGGTCCAGGCGACGAGCGGCCGCCGCAGCAGCCGCGGCCA
TTAGTGCTATGACTCAGGGTCGCAGGGGCAACGTGTATTGGGTGCGCGACTCGGTTAGCGGCCTGC
GCGTGCCCGTGCGCACCCGCCCCCCGCGCAACTAGATTGCAAGAAAAAACTACTTAGACTCGTACT
GTTGTATGTATCCAGCGGCGGCGGCGCGCAACGAAGCTATGTCCAAGCGCAAAATCAAAGAAGAG
ATGCTCCAGGTCATCGCGCCGGAGATCTATGGCCCCCCGAAGAAGGAAGAGCAGGATTACAAGCC
CCGAAAGCTAAAGCGGGTCAAAAAGAAAAAGAAAGATGATGATGATGAACTTGACGACGAGGTG
GAACTGCTGCACGCTACCGCGCCCAGGCGACGGGTACAGTGGAAAGGTCGACGCGTAAAACGTGT
TTTGCGACCCGGCACCACCGTAGTCTTTACGCCCGGTGAGCGCTCCACCCGCACCTACAAGCGCGT
GTATGATGAGGTGTACGGCGACGAGGACCTGCTTGAGCAGGCCAACGAGCGCCTCGGGGAGTTTG
CCTACGGAAAGCGGCATAAGGACATGCTGGCGTTGCCGCTGGACGAGGGCAACCCAACACCTAGC
CTAAAGCCCGTAACACTGCAGCAGGTGCTGCCCGCGCTTGCACCGTCCGAAGAAAAGCGCGGCCT
AAAGCGCGAGTCTGGTGACTTGGCACCCACCGTGCAGCTGATGGTACCCAAGCGCCAGCGACTGG
AAGATGTCTTGGAAAAAATGACCGTGGAACCTGGGCTGGAGCCCGAGGTCCGCGTGCGGCCAATC
AAGCAGGTGGCGCCGGGACTGGGCGTGCAGACCGTGGACGTTCAGATACCCACTACCAGTAGCAC
CAGTATTGCCACCGCCACAGAGGGCATGGAGACACAAACGTCCCCGGTTGCCTCAGCGGTGGCGG
ATGCCGCGGTGCAGGCGGTCGCTGCGGCCGCGTCCAAGACCTCTACGGAGGTGCAAACGGACCCG
TGGATGTTTCGCGTTTCAGCCCCCCGGCGCCCGCGCCGTTCGAGGAAGTACGGCGCCGCCAGCGCG
CTACTGCCCGAATATGCCCTACATCCTTCCATTGCGCCTACCCCCGGCTATCGTGGCTACACCTACC
GCCCCAGAAGACGAGCAACTACCCGACGCCGAACCACCACTGGAACCCGCCGCCGCCGTCGCCGT
CGCCAGCCCGTGCTGGCCCCGATTTCCGTGCGCAGGGTGGCTCGCGAAGGAGGCAGGACCCTGGT
GCTGCCAACAGCGCGCTACCACCCCAGCATCGTTTAAAAGCCGGTCTTTGTGGTTCTTGCAGATAT
GGCCCTCACCTGCCGCCTCCGTTTCCCGGTGCCGGGATTCCGAGGAAGAATGCACCGTAGGAGGG
GCATGGCCGGCCACGGCCTGACGGGCGGCATGCGTCGTGCGCACCACCGGCGGCGGCGCGCGTCG
CACCGTCGCATGCGCGGCGGTATCCTGCCCCTCCTTATTCCACTGATCGCCGCGGCGATTGGCGCC
GTGCCCGGAATTGCATCCGTGGCCTTGCAGGCGCAGAGACACTGATTAAAAACAAGTTGCATGTG
GAAAAATCAAAATAAAAAGTCTGGACTCTCACGCTCGCTTGGTCCTGTAACTATTTTGTAGAATGG
AAGACATCAACTTTGCGTCTCTGGCCCCGCGACACGGCTCGCGCCCGTTCATGGGAAACTGGCAAG
ATATCGGCACCAGCAATATGAGCGGTGGCGCCTTCAGCTGGGGCTCGCTGTGGAGCGGCATTAAA
AATTTCGGTTCCACCGTTAAGAACTATGGCAGCAAGGCCTGGAACAGCAGCACAGGCCAGATGCT
GAGGGATAAGTTGAAAGAGCAAAATTTCCAACAAAAGGTGGTAGATGGCCTGGCCTCTGGCATTA
GCGGGGTGGTGGACCTGGCCAACCAGGCAGTGCAAAATAAGATTAACAGTAAGCTTGATCCCCGC
CCTCCCGTAGAGGAGCCTCCACCGGCCGTGGAGACAGTGTCTCCAGAGGGGCGTGGCGAAAAGCG
TCCGCGCCCCGACAGGGAAGAAACTCTGGTGACGCAAATAGACGAGCCTCCCTCGTACGAGGAGG
CACTAAAGCAAGGCCTGCCCACCACCCGTCCCATCGCGCCCATGGCTACCGGAGTGCTGGGCCAG
CACACACCCGTAACGCTGGACCTGCCTCCCCCCGCCGACACCCAGCAGAAACCTGTGCTGCCAGG
CCCGACCGCCGTTGTTGTAACCCGTCCTAGCCGCGCGTCCCTGCGCCGCGCCGCCAGCGGTCCGCG
ATCGTTGCGGCCCGTAGCCAGTGGCAACTGGCAAAGCACACTGAACAGCATCGTGGGTCTGGGGG
TGCAATCCCTGAAGCGCCGACGATGCTTCTGATAGCTAACGTGTCGTATGTGTGTCATGTATGCGT
CCATGTCGCCGCCAGAGGAGCTGCTGAGCCGCCGCGCGCCCGCTTTCCAAGATGGCTACCCCTTCG
ATGATGCCGCAGTGGTCTTACATGCACATCTCGGGCCAGGACGCCTCGGAGTACCTGAGCCCCGG
GCTGGTGCAGTTTGCCCGCGCCACCGAGACGTACTTCAGCCTGAATAACAAGTTTAGAAACCCCAC
GGTGGCGCCTACGCACGACGTGACCACAGACCGGTCCCAGCGTTTGACGCTGCGGTTCATCCCTGT
GGACCGTGAGGATACTGCGTACTCGTACAAGGCGCGGTTCACCCTAGCTGTGGGTGATAACCGTGT
GCTGGACATGGCTTCCACGTACTTTGACATCCGCGGCGTGCTGGACAGGGGCCCTACTTTTAAGCC
CTACTCTGGCACTGCCTACAACGCCCTGGCTCCCAAGGGTGCCCCAAATCCTTGCGAATGGGATGA
AGCTGCTACTGCTCTTGAAATAAACCTAGAAGAAGAGGACGATGACAACGAAGACGAAGTAGAC
GAGCAAGCTGAGCAGCAAAAAACTCACGTATTTGGGCAGGCGCCTTATTCTGGTATAAATATTAC
AAAGGAGGGTATTCAAATAGGTGTCGAAGGTCAAACACCTAAATATGCCGATAAAACATTTCAAC
CTGAACCTCAAATAGGAGAATCTCAGTGGTACGAAACAGAAATTAATCATGCAGCTGGGAGAGTC
CTAAAAAAGACTACCCCAATGAAACCATGTTACGGTTCATATGCAAAACCCACAAATGAAAATGG
AGGGCAAGGCATTCTTGTAAAGCAACAAAATGGAAAGCTAGAAAGTCAAGTGGAAATGCAATTTT
TCTCAACTACTGAGGCAGCCGCAGGCAATGGTGATAACTTGACTCCTAAAGTGGTATTGTACAGTG
AAGATGTAGATATAGAAACCCCAGACACTCATATTTCTTACATGCCCACTATTAAGGAAGGTAACT
CACGAGAACTAATGGGCCAACAATCTATGCCCAACAGGCCTAATTACATTGCTTTTAGGGACAATT
TTATTGGTCTAATGTATTACAACAGCACGGGTAATATGGGTGTTCTGGCGGGCCAAGCATCGCAGT
TGAATGCTGTTGTAGATTTGCAAGACAGAAACACAGAGCTTTCATACCAGCTTTTGCTTGATTCCA
TTGGTGATAGAACCAGGTACTTTTCTATGTGGAATCAGGCTGTTGACAGCTATGATCCAGATGTTA
GAATTATTGAAAATCATGGAACTGAAGATGAACTTCCAAATTACTGCTTTCCACTGGGAGGTGTGA
TTAATACAGAGACTCTTACCAAGGTAAAACCTAAAACAGGTCAGGAAAATGGATGGGAAAAAGAT
GCTACAGAATTTTCAGATAAAAATGAAATAAGAGTTGGAAATAATTTTGCCATGGAAATCAATCT
AAATGCCAACCTGTGGAGAAATTTCCTGTACTCCAACATAGCGCTGTATTTGCCCGACAAGCTAAA
GTACAGTCCTTCCAACGTAAAAATTTCTGATAACCCAAACACCTACGACTACATGAACAAGCGAGT
GGTGGCTCCCGGGCTAGTGGACTGCTACATTAACCTTGGAGCACGCTGGTCCCTTGACTATATGGA
CAACGTCAACCCATTTAACCACCACCGCAATGCTGGCCTGCGCTACCGCTCAATGTTGCTGGGCAA
TGGTCGCTATGTGCCCTTCCACATCCAGGTGCCTCAGAAGTTCTTTGCCATTAAAAACCTCCTTCTC
CTGCCGGGCTCATACACCTACGAGTGGAACTTCAGGAAGGATGTTAACATGGTTCTGCAGAGCTCC
CTAGGAAATGACCTAAGGGTTGACGGAGCCAGCATTAAGTTTGATAGCATTTGCCTTTACGCCACC
TTCTTCCCCATGGCCCACAACACCGCCTCCACGCTTGAGGCCATGCTTAGAAACGACACCAACGAC
CAGTCCTTTAACGACTATCTCTCCGCCGCCAACATGCTCTACCCTATACCCGCCAACGCTACCAAC
GTGCCCATATCCATCCCCTCCCGCAACTGGGCGGCTTTCCGCGGCTGGGCCTTCACGCGCCTTAAG
ACTAAGGAAACCCCATCACTGGGCTCGGGCTACGACCCTTATTACACCTACTCTGGCTCTATACCC
TACCTAGATGGAACCTTTTACCTCAACCACACCTTTAAGAAGGTGGCCATTACCTTTGACTCTTCTG
TCAGCTGGCCTGGCAATGACCGCCTGCTTACCCCCAACGAGTTTGAAATTAAGCGCTCAGTTGACG
GGGAGGGTTACAACGTTGCCCAGTGTAACATGACCAAAGACTGGTTCCTGGTACAAATGCTAGCT
AACTATAACATTGGCTACCAGGGCTTCTATATCCCAGAGAGCTACAAGGACCGCATGTACTCCTTC
TTTAGAAACTTCCAGCCCATGAGCCGTCAGGTGGTGGATGATACTAAATACAAGGACTACCAACA
GGTGGGCATCCTACACCAACACAACAACTCTGGATTTGTTGGCTACCTTGCCCCCACCATGCGCGA
AGGACAGGCCTACCCTGCTAACTTCCCCTATCCGCTTATAGGCAAGACCGCAGTTGACAGCATTAC
CCAGAAAAAGTTTCTTTGCGATCGCACCCTTTGGCGCATCCCATTCTCCAGTAACTTTATGTCCATG
GGCGCACTCACAGACCTGGGCCAAAACCTTCTCTACGCCAACTCCGCCCACGCGCTAGACATGACT
TTTGAGGTGGATCCCATGGACGAGCCCACCCTTCTTTATGTTTTGTTTGAAGTCTTTGACGTGGTCC
GTGTGCACCAGCCGCACCGCGGCGTCATCGAAACCGTGTACCTGCGCACGCCCTTCTCGGCCGGCA
ACGCCACAACATAAAGAAGCAAGCAACATCAACAACAGCTGCCGCCATGGGCTCCAGTGAGCAG
GAACTGAAAGCCATTGTCAAAGATCTTGGTTGTGGGCCATATTTTTTGGGCACCTATGACAAGCGC
TTTCCAGGCTTTGTTTCTCCACACAAGCTCGCCTGCGCCATAGTCAATACGGCCGGTCGCGAGACT
GGGGGCGTACACTGGATGGCCTTTGCCTGGAACCCGCACTCAAAAACATGCTACCTCTTTGAGCCC
TTTGGCTTTTCTGACCAGCGACTCAAGCAGGTTTACCAGTTTGAGTACGAGTCACTCCTGCGCCGT
AGCGCCATTGCTTCTTCCCCCGACCGCTGTATAACGCTGGAAAAGTCCACCCAAAGCGTACAGGGG
CCCAACTCGGCCGCCTGTGGACTATTCTGCTGCATGTTTCTCCACGCCTTTGCCAACTGGCCCCAAA
CTCCCATGGATCACAACCCCACCATGAACCTTATTACCGGGGTACCCAACTCCATGCTCAACAGTC
CCCAGGTACAGCCCACCCTGCGTCGCAACCAGGAACAGCTCTACAGCTTCCTGGAGCGCCACTCGC
CCTACTTCCGCAGCCACAGTGCGCAGATTAGGAGCGCCACTTCTTTTTGTCACTTGAAAAACATGT
AAAAATAATGTACTAGAGACACTTTCAATAAAGGCAAATGCTTTTATTTGTACACTCTCGGGTGAT
TATTTACCCCCACCCTTGCCGTCTGCGCCGTTTAAAAATCAAAGGGGTTCTGCCGCGCATCGCTAT
GCGCCACTGGCAGGGACACGTTGCGATACTGGTGTTTAGTGCTCCACTTAAACTCAGGCACAACCA
TCCGCGGCAGCTCGGTGAAGTTTTCACTCCACAGGCTGCGCACCATCACCAACGCGTTTAGCAGGT
CGGGCGCCGATATCTTGAAGTCGCAGTTGGGGCCTCCGCCCTGCGCGCGCGAGTTGCGATACACA
GGGTTGCAGCACTGGAACACTATCAGCGCCGGGTGGTGCACGCTGGCCAGCACGCTCTTGTCGGA
GATCAGATCCGCGTCCAGGTCCTCCGCGTTGCTCAGGGCGAACGGAGTCAACTTTGGTAGCTGCCT
TCCCAAAAAGGGCGCGTGCCCAGGCTTTGAGTTGCACTCGCACCGTAGTGGCATCAAAAGGTGAC
CGTGCCCGGTCTGGGCGTTAGGATACAGCGCCTGCATAAAAGCCTTGATCTGCTTAAAAGCCACCT
GAGCCTTTGCGCCTTCAGAGAAGAACATGCCGCAAGACTTGCCGGAAAACTGATTGGCCGGACAG
GCCGCGTCGTGCACGCAGCACCTTGCGTCGGTGTTGGAGATCTGCACCACATTTCGGCCCCACCGG
TTCTTCACGATCTTGGCCTTGCTAGACTGCTCCTTCAGCGCGCGCTGCCCGTTTTCGCTCGTCACAT
CCATTTCAATCACGTGCTCCTTATTTATCATAATGCTTCCGTGTAGACACTTAAGCTCGCCTTCGAT
CTCAGCGCAGCGGTGCAGCCACAACGCGCAGCCCGTGGGCTCGTGATGCTTGTAGGTCACCTCTGC
AAACGACTGCAGGTACGCCTGCAGGAATCGCCCCATCATCGTCACAAAGGTCTTGTTGCTGGTGAA
GGTCAGCTGCAACCCGCGGTGCTCCTCGTTCAGCCAGGTCTTGCATACGGCCGCCAGAGCTTCCAC
TTGGTCAGGCAGTAGTTTGAAGTTCGCCTTTAGATCGTTATCCACGTGGTACTTGTCCATCAGCGCG
CGCGCAGCCTCCATGCCCTTCTCCCACGCAGACACGATCGGCACACTCAGCGGGTTCATCACCGTA
ATTTCACTTTCCGCTTCGCTGGGCTCTTCCTCTTCCTCTTGCGTCCGCATACCACGCGCCACTGGGT
CGTCTTCATTCAGCCGCCGCACTGTGCGCTTACCTCCTTTGCCATGCTTGATTAGCACCGGTGGGTT
GCTGAAACCCACCATTTGTAGCGCCACATCTTCTCTTTCTTCCTCGCTGTCCACGATTACTTTCACA
GGAGGTACAGCTATGACCATGATTACGGATTCACTGGCCGTCGTTTTACAACGTCGTGACTGGGAA
AACCCTGGCGTTACCCAACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGGCGAATAGGTCGCGCC
GCACCGCGTCCGCGCTCGGGGGTGGTTTCGCGCTGCTCCTCTTCCCGACTGGCCATTTCCTTCTCCT
ATAGGCAGAAAAAGATCATGGAGTCAGTCGAGAAGAAGGACAGCCTAACCGCCCCCTCTGAGTTC
GCCACCACCGCCTCCACCGATGCCGCCAACGCGCCTACCACCTTCCCCGTCGAGGCACCCCCGCTT
GAGGAGGAGGAAGTGATTATCGAGCAGGACCCAGGTTTTGTAAGCGAAGACGACGAGGACCGCT
CAGTACCAACAGAGGATAAAAAGCAAGACCAGGACAACGCAGAGGCAAACGAGGAACAAGTCGG
GCGGGGGGACGAAAGGCATGGCGACTACCTAGATGTGGGAGACGACGTGCTGTTGAAGCATCTGC
AGCGCCAGTGCGCCATTATCTGCGACGCGTTGCAAGAGCGCAGCGATGTGCCCCTCGCCATAGCG
GATGTCAGCCTTGCCTACGAACGCCACCTATTCTCACCGCGCGTACCCCCCAAACGCCAAGAAAAC
GGCACATGCGAGCCCAACCCGCGCCTCAACTTCTACCCCGTATTTGCCGTGCCAGAGGTGCTTGCC
ACCTATCACATCTTTTTCCAAAACTGCAAGATACCCCTATCCTGCCGTGCCAACCGCAGCCGAGCG
GACAAGCAGCTGGCCTTGCGGCAGGGCGCTGTCATACCTGATATCGCCTCGCTCAACGAAGTGCC
AAAAATCTTTGAGGGTCTTGGACGCGACGAGAAGCGCGCGGCAAACGCTCTGCAACAGGAAAACA
GCGAAAATGAAAGTCACTCTGGAGTGTTGGTGGAACTCGAGGGTGACAACGCGCGCCTAGCCGTA
CTAAAACGCAGCATCGAGGTCACCCACTTTGCCTACCCGGCACTTAACCTACCCCCCAAGGTCATG
AGCACAGTCATGAGTGAGCTGATCGTGCGCCGTGCGCAGCCCCTGGAGAGGGATGCAAATTTGCA
AGAACAAACAGAGGAGGGCCTACCCGCAGTTGGCGACGAGCAGCTAGCGCGCTGGCTTCAAACGC
GCGAGCCTGCCGACTTGGAGGAGCGACGCAAACTAATGATGGCCGCAGTGCTCGTTACCGTGGAG
CTTGAGTGCATGCAGCGGTTCTTTGCTGACCCGGAGATGCAGCGCAAGCTAGAGGAAACATTGCA
CTACACCTTTCGACAGGGCTACGTACGCCAGGCCTGCAAGATCTCCAACGTGGAGCTCTGCAACCT
GGTCTCCTACCTTGGAATTTTGCACGAAAACCGCCTTGGGCAAAACGTGCTTCATTCCACGCTCAA
GGGCGAGGCGCGCCGCGACTACGTCCGCGACTGCGTTTACTTATTTCTATGCTACACCTGGCAGAC
GGCCATGGGCGTTTGGCAGCAGTGCTTGGAGGAGTGCAACCTCAAGGAGCTGCAGAAACTGCTAA
AGCAAAACTTGAAGGACCTATGGACGGCCTTCAACGAGCGCTCCGTGGCCGCGCACCTGGCGGAC
ATCATTTTCCCCGAACGCCTGCTTAAAACCCTGCAACAGGGTCTGCCAGACTTCACCAGTCAAAGC
ATGTTGCAGAACTTTAGGAACTTTATCCTAGAGCGCTCAGGAATCTTGCCCGCCACCTGCTGTGCA
CTTCCTAGCGACTTTGTGCCCATTAAGTACCGCGAATGCCCTCCGCCGCTTTGGGGCCACTGCTACC
TTCTGCAGCTAGCCAACTACCTTGCCTACCACTCTGACATAATGGAAGACGTGAGCGGTGACGGTC
TACTGGAGTGTCACTGTCGCTGCAACCTATGCACCCCGCACCGCTCCCTGGTTTGCAATTCGCAGC
TGCTTAACGAAAGTCAAATTATCGGTACCTTTGAGCTGCAGGGTCCCTCGCCTGACGAAAAGTCCG
CGGCTCCGGGGTTGAAACTCACTCCGGGGCTGTGGACGTCGGCTTACCTTCGCAAATTTGTACCTG
AGGACTACCACGCCCACGAGATTAGGTTCTACGAAGACCAATCCCGCCCGCCTAATGCGGAGCTT
ACCGCCTGCGTCATTACCCAGGGCCACATTCTTGGCCAATTGCAAGCCATCAACAAAGCCCGCCAA
GAGTTTCTGCTACGAAAGGGACGGGGGGTTTACTTGGACCCCCAGTCCGGCGAGGAGCTCAACCC
AATCCCCCCGCCGCCGCAGCCCTATCAGCAGCAGCCGCGGGCCCTTGCTTCCCAGGATGGCACCCA
AAAAGAAGCTGCAGCTGCCGCCGCCACCCACGGACGAGGAGGAATACTGGGACAGTCAGGCAGA
GGAGGTTTTGGACGAGGAGGAGGAGGACATGATGGAAGACTGGGAGAGCCTAGACGAGGAAGCT
TCCGAGGTCGAAGAGGTGTCAGACGAAACACCGTCACCCTCGGTCGCATTCCCCTCGCCGGCGCCC
CAGAAATCGGCAACCGGTTCCAGCATGGCTACAACCTCCGCTCCTCAGGCGCCGCCGGCACTGCCC
GTTCGCCGACCCAACCGTAGATGGGACACCACTGGAACCAGGGCCGGTAAGTCCAAGCAGCCGCC
GCCGTTAGCCCAAGAGCAACAACAGCGCCAAGGCTACCGCTCATGGCGCGGGCACAAGAACGCCA
TAGTTGCTTGCTTGCAAGACTGTGGGGGCAACATCTCCTTCGCCCGCCGCTTTCTTCTCTACCATCA
CGGCGTGGCCTTCCCCCGTAACATCCTGCATTACTACCGTCATCTCTACAGCCCATACTGCACCGG
CGGCAGCGGCAGCAACAGCAGCGGCCACACAGAAGCAAAGGCGACCGGATAGCAAGACTCTGAC
AAAGCCCAAGAAATCCACAGCGGCGGCAGCAGCAGGAGGAGGAGCGCTGCGTCTGGCGCCCAAC
GAACCCGTATCGACCCGCGAGCTTAGAAACAGGATTTTTCCCACTCTGTATGCTATATTTCAACAG
AGCAGGGGCCAAGAACAAGAGCTGAAAATAAAAAACAGGTCTCTGCGATCCCTCACCCGCAGCTG
CCTGTATCACAAAAGCGAAGATCAGCTTCGGCGCACGCTGGAAGACGCGGAGGCTCTCTTCAGTA
AATACTGCGCGCTGACTCTTAAGGACTAGTTTCGCGCCCTTTCTCAAATTTAAGCGCGAAAACTAC
GTCATCTCCAGCGGCCACACCCGGCGCCAGCACCTGTTGTCAGCGCCATTATGAGCAAGGAAATTC
CCACGCCCTACATGTGGAGTTACCAGCCACAAATGGGACTTGCGGCTGGAGCTGCCCAAGACTAC
TCAACCCGAATAAACTACATGAGCGCGGGACCCCACATGATATCCCGGGTCAACGGAATACGCGC
CCACCGAAACCGAATTCTCCTGGAACAGGCGGCTATTACCACCACACCTCGTAATAACCTTAATCC
CCGTAGTTGGCCCGCTGCCCTGGTGTACCAGGAAAGTCCCGCTCCCACCACTGTGGTACTTCCCAG
AGACGCCCAGGCCGAAGTTCAGATGACTAACTCAGGGGCGCAGCTTGCGGGCGGCTTTCGTCACA
GGGTGCGGTCGCCCGGGCAGGGTATAACTCACCTGACAATCAGAGGGCGAGGTATTCAGCTCAAC
GACGAGTCGGTGAGCTCCTCGCTTGGTCTCCGTCCGGACGGGACATTTCAGATCGGCGGCGCCGGC
CGCTCTTCATTCACGCCTCGTCAGGCAATCCTAACTCTGCAGACCTCGTCCTCTGAGCCGCGCTCTG
GAGGCATTGGAACTCTGCAATTTATTGAGGAGTTTGTGCCATCGGTCTACTTTAACCCCTTCTCGGG
ACCTCCCGGCCACTATCCGGATCAATTTATTCCTAACTTTGACGCGGTAAAGGACTCGGCGGACGG
CTACGACTGAATGTTAAGTGGAGAGGCAGAGCAACTGCGCCTGAAACACCTGGTCCACTGTCGCC
GCCACAAGTGCTTTGCCCGCGACTCCGGTGAGTTTTGCTACTTTGAATTGCCCGAGGATCATATCG
AGGGCCCGGCGCACGGCGTCCGGCTTACCGCCCAGGGAGAGCTTGCCCGTAGCCTGATTCGGGAG
TTTACCCAGCGCCCCCTGCTAGTTGAGCGGGACAGGGGACCCTGTGTTCTCACTGTGATTTGCAAC
TGTCCTAACCCTGGATTACATCAAGATCTTTGTTGCCATCTCTGTGCTGAGTATAATAAATACAGA
AATTAAAATATACTGGGGCTCCTATCGCCATCCTGTAAACGCCACCGTCTTCACCCGCCCAAGCAA
ACCAAGGCGAACCTTACCTGGTACTTTTAACATCTCTCCCTCTGTGATTTACAACAGTTTCAACCCA
GACGGAGTGAGTCTACGAGAGAACCTCTCCGAGCTCAGCTACTCCATCAGAAAAAACACCACCCT
CCTTACCTGCCGGGAACGTACGAGTGCGTCACCGGCCGCTGCACCACACCTACCGCCTGACCGTAA
ACCAGACTTTTTCCGGACAGACCTCAATAACTCTGTTTACCAGAACAGGAGGTGAGCTTAGAAAAC
CCTTAGGGTATTAGGCCAAAGGCGCAGCTACTGTGGGGTTTATGAACAATTCAAGCAACTCTACGG
GCTATTCTAATTCAGGTTTCTCTAGAAATGGACGGAATTATTACAGAGCAGCGCCTGCTAGAAAGA
CGCAGGGCAGCGGCCGAGCAACAGCGCATGAATCAAGAGCTCCAAGACATGGTTAACTTGCACCA
GTGCAAAAGGGGTATCTTTTGTCTGGTAAAGCAGGCCAAAGTCACCTACGACAGTAATACCACCG
GACACCGCCTTAGCTACAAGTTGCCAACCAAGCGTCAGAAATTGGTGGTCATGGTGGGAGAAAAG
CCCATTACCATAACTCAGCACTCGGTAGAAACCGAAGGCTGCATTCACTCACCTTGTCAAGGACCT
GAGGATCTCTGCACCCTTATTAAGACCCTGTGCGGTCTCAAAGATCTTATTCCCTTTAACTAATAAA
AAAAAATAATAAAGCATCACTTACTTAAAATCAGTTAGCAAATTTCTGTCCAGTTTATTCAGCAGC
ACCTCCTTGCCCTCCTCCCAGCTCTGGTATTGCAGCTTCCTCCTGGCTGCAAACTTTCTCCACAATC
TAAATGGAATGTCAGTTTCCTCCTGTTCCTGTCCATCCGCACCCACTATCTTCATGTTGTTGCAGAT
GAAGCGCGCAAGACCGTCTGAAGATACCTTCAACCCCGTGTATCCATATGACACGGAAACCGGTC
CTCCAACTGTGCCTTTTCTTACTCCTCCCTTTGTATCCCCCAATGGGTTTCAAGAGAGTCCCCCTGG
GGTACTCTCTTTGCGCCTATCCGAACCTCTAGTTACCTCCAATGGCATGCTTGCGCTCAAAATGGGC
AACGGCCTCTCTCTGGACGAGGCCGGCAACCTTACCTCCCAAAATGTAACCACTGTGAGCCCACCT
CTCAAAAAAACCAAGTCAAACATAAACCTGGAAATATCTGCACCCCTCACAGTTACCTCAGAAGC
CCTAACTGTGGCTGCCGCCGCACCTCTAATGGTCGCGGGCAACACACTCACCATGCAATCACAGGC
CCCGCTAACCGTGCACGACTCCAAACTTAGCATTGCCACCCAAGGACCCCTCACAGTGTCAGAAG
GAAAGCTAGCCCTGCAAACATCAGGCCCCCTCACCACCACCGATAGCAGTACCCTTACTATCACTG
CCTCACCCCCTCTAACTACTGCCACTGGTAGCTTGGGCATTGACTTGAAAGAGCCCATTTATACAC
AAAATGGAAAACTAGGACTAAAGTACGGGGCTCCTTTGCATGTAACAGACGACCTAAACACTTTG
ACCGTAGCAACTGGTCCAGGTGTGACTATTAATAATACTTCCTTGCAAACTAAAGTTACTGGAGCC
TTGGGTTTTGATTCACAAGGCAATATGCAACTTAATGTAGCAGGAGGACTAAGGATTGATTCTCAA
AACAGACGCCTTATACTTGATGTTAGTTATCCGTTTGATGCTCAAAACCAACTAAATCTAAGACTA
GGACAGGGCCCTCTTTTTATAAACTCAGCCCACAACTTGGATATTAACTACAACAAAGGCCTTTAC
TTGTTTACAGCTTCAAACAATTCCAAAAAGCTTGAGGTTAACCTAAGCACTGCCAAGGGGTTGATG
TTTGACGCTACAGCCATAGCCATTAATGCAGGAGATGGGCTTGAATTTGGTTCACCTAATGCACCA
AACACAAATCCCCTCAAAACAAAAATTGGCCATGGCCTAGAATTTGATTCAAACAAGGCTATGGT
TCCTAAACTAGGAACTGGCCTTAGTTTTGACAGCACAGGTGCCATTACAGTAGGAAACAAAAATA
ATGATAAGCTAACTTTGTGGACCACACCAGCTCCATCTCCTAACTGTAGACTAAATGCAGAGAAAG
ATGCTAAACTCACTTTGGTCTTAACAAAATGTGGCAGTCAAATACTTGCTACAGTTTCAGTTTTGGC
TGTTAAAGGCAGTTTGGCTCCAATATCTGGAACAGTTCAAAGTGCTCATCTTATTATAAGATTTGA
CGAAAATGGAGTGCTACTAAACAATTCCTTCCTGGACCCAGAATATTGGAACTTTAGAAATGGAG
ATCTTACTGAAGGCACAGCCTATACAAACGCTGTTGGATTTATGCCTAACCTATCAGCTTATCCAA
AATCTCACGGTAAAACTGCCAAAAGTAACATTGTCAGTCAAGTTTACTTAAACGGAGACAAAACT
AAACCTGTAACACTAACCATTACACTAAACGGTACACAGGAAACAGGAGACACAACTCCAAGTGC
ATACTCTATGTCATTTTCATGGGACTGGTCTGGCCACAACTACATTAATGAAATATTTGCCACATCC
TCTTACACTTTTTCATACATTGCCCAAGAATAAAGAATCGTTTGTGTTATGTTTCAACGTGTTTATT
TTTCAATTGCAGAAAATTTCGAATCATTTTTCATTCAGTAGTATAGCCCCACCACCACATAGCTTAT
ACAGATCACCGTACCTTAATCAAACTCACAGAACCCTAGTATTCAACCTGCCACCTCCCTCCCAAC
ACACAGAGTACACAGTCCTTTCTCCCCGGCTGGCCTTAAAAAGCATCATATCATGGGTAACAGACA
TATTCTTAGGTGTTATATTCCACACGGTTTCCTGTCGAGCCAAACGCTCATCAGTGATATTAATAAA
CTCCCCGGGCAGCTCACTTAAGTTCATGTCGCTGTCCAGCTGCTGAGCCACAGGCTGCTGTCCAAC
TTGCGGTTGCTTAACGGGCGGCGAAGGAGAAGTCCACGCCTACATGGGGGTAGAGTCATAATCGT
GCATCAGGATAGGGCGGTGGTGCTGCAGCAGCGCGCGAATAAACTGCTGCCGCCGCCGCTCCGTC
CTGCAGGAATACAACATGGCAGTGGTCTCCTCAGCGATGATTCGCACCGCCCGCAGCATAAGGCG
CCTTGTCCTCCGGGCACAGCAGCGCACCCTGATCTCACTTAAATCAGCACAGTAACTGCAGCACAG
CACCACAATATTGTTCAAAATCCCACAGTGCAAGGCGCTGTATCCAAAGCTCATGGCGGGGACCA
CAGAACCCACGTGGCCATCATACCACAAGCGCAGGTAGATTAAGTGGCGACCCCTCATAAACACG
CTGGACATAAACATTACCTCTTTTGGCATGTTGTAATTCACCACCTCCCGGTACCATATAAACCTCT
GATTAAACATGGCGCCATCCACCACCATCCTAAACCAGCTGGCCAAAACCTGCCCGCCGGCTATAC
ACTGCAGGGAACCGGGACTGGAACAATGACAGTGGAGAGCCCAGGACTCGTAACCATGGATCATC
ATGCTCGTCATGATATCAATGTTGGCACAACACAGGCACACGTGCATACACTTCCTCAGGATTACA
AGCTCCTCCCGCGTTAGAACCATATCCCAGGGAACAACCCATTCCTGAATCAGCGTAAATCCCACA
CTGCAGGGAAGACCTCGCACGTAACTCACGTTGTGCATTGTCAAAGTGTTACATTCGGGCAGCAGC
GGATGATCCTCCAGTATGGTAGCGCGGGTTTCTGTCTCAAAAGGAGGTAGACGATCCCTACTGTAC
GGAGTGCGCCGAGACAACCGAGATCGTGTTGGTCGTAGTGTCATGCCAAATGGAACGCCGGACGT
AGTCATATTTCCTGAAGCAAAACCAGGTGCGGGCGTGACAAACAGATCTGCGTCTCCGGTCTCGCC
GCTTAGATCGCTCTGTGTAGTAGTTGTAGTATATCCACTCTCTCAAAGCATCCAGGCGCCCCCTGG
CTTCGGGTTCTATGTAAACTCCTTCATGCGCCGCTGCCCTGATAACATCCACCACCGCAGAATAAG
CCACACCCAGCCAACCTACACATTCGTTCTGCGAGTCACACACGGGAGGAGCGGGAAGAGCTGGA
AGAACCATGTTTTTTTTTTTATTCCAAAAGATTATCCAAAACCTCAAAATGAAGATCTATTAAGTG
AACGCGCTCCCCTCCGGTGGCGTGGTCAAACTCTACAGCCAAAGAACAGATAATGGCATTTGTAA
GATGTTGCACAATGGCTTCCAAAAGGCAAACGGCCCTCACGTCCAAGTGGACGTAAAGGCTAAAC
CCTTCAGGGTGAATCTCCTCTATAAACATTCCAGCACCTTCAACCATGCCCAAATAATTCTCATCTC
GCCACCTTCTCAATATATCTCTAAGCAAATCCCGAATATTAAGTCCGGCCATTGTAAAAATCTGCT
CCAGAGCGCCCTCCACCTTCAGCCTCAAGCAGCGAATCATGATTGCAAAAATTCAGGTTCCTCACA
GACCTGTATAAGATTCAAAAGCGGAACATTAACAAAAATACCGCGATCCCGTAGGTCCCTTCGCA
GGGCCAGCTGAACATAATCGTGCAGGTCTGCACGGACCAGCGCGGCCACTTCCCCGCCAGGAACC
ATGACAAAAGAACCCACACTGATTATGACACGCATACTCGGAGCTATGCTAACCAGCGTAGCCCC
GATGTAAGCTTGTTGCATGGGCGGCGATATAAAATGCAAGGTGCTGCTCAAAAAATCAGGCAAAG
CCTCGCGCAAAAAAGAAAGCACATCGTAGTCATGCTCATGCAGATAAAGGCAGGTAAGCTCCGGA
ACCACCACAGAAAAAGACACCATTTTTCTCTCAAACATGTCTGCGGGTTTCTGCATAAACACAAAA
TAAAATAACAAAAAAACATTTAAACATTAGAAGCCTGTCTTACAACAGGAAAAACAACCCTTATA
AGCATAAGACGGACTACGGCCATGCCGGCGTGACCGTAAAAAAACTGGTCACCGTGATTAAAAAG
CACCACCGACAGCTCCTCGGTCATGTCCGGAGTCATAATGTAAGACTCGGTAAACACATCAGGTTG
ATTCACATCGGTCAGTGCTAAAAAGCGACCGAAATAGCCCGGGGGAATACATACCCGCAGGCGTA
GAGACAACATTACAGCCCCCATAGGAGGTATAACAAAATTAATAGGAGAGAAAAACACATAAAC
ACCTGAAAAACCCTCCTGCCTAGGCAAAATAGCACCCTCCCGCTCCAGAACAACATACAGCGCTTC
CACAGCGGCAGCCATAACAGTCAGCCTTACCAGTAAAAAAGAAAACCTATTAAAAAAACACCACT
CGACACGGCACCAGCTCAATCAGTCACAGTGTAAAAAAGGGCCAAGTGCAGAGCGAGTATATATA
GGACTAAAAAATGACGTAACGGTTAAAGTCCACAAAAAACACCCAGAAAACCGCACGCGAACCT
ACGCCCAGAAACGAAAGCCAAAAAACCCACAACTTCCTCAAATCGTCACTTCCGTTTTCCCACGTT
ACGTCACTTCCCATTTTAAGAAAACTACAATTCCCAACACATACAAGTTACTCCGCCCTAAAACCT
ACGTCACCCGCCCCGTTCCCACGCCCCGCGCCACGTCACAAACTCCACCCCCTCATTATCATATTG
GCTTCAATCCAAAATAAGGTATATTATTGATGATGTTAATTAATTTAAATCCGCATGCGATATCGA
GCTCTCCCGGGAATTCGGATCTGCGACGCGAGGCTGGATGGCCTTCCCCATTATGATTCTTCTCGC
GTTTAAGGGCACCAATAACTGCCTTAAAAAAATTACGCCCCGCCCTGCCACTCATCGCAGTACTGT
TGTAATTCATTAAGCATTCTGCCGACATGGAAGCCATCACAAACGGCATGATGAACCTGAATCGCC
AGCGGCATCAGCACCTTGTCGCCTTGCGTATAATATTTGCCCATGGTGAAAACGGGGGCGAAGAA
GTTGTCCATATTGGCCACGTTTAAATCAAAACTGGTGAAACTCACCCAGGGATTGGCTGAGACGAA
AAACATATTCTCAATAAACCCTTTAGGGAAATAGGCCAGGTTTTCACCGTAACACGCCACATCTTG
CGAATATATGTGTAGAAACTGCCGGAAATCGTCGTGGTATTCACTCCAGAGCGATGAAAACGTTTC
AGTTTGCTCATGGAAAACGGTGTAACAAGGGTGAACACTATCCCATATCACCAGCTCACCGTCTTT
CATTGCCATACGGAATTCCGGATGAGCATTCATCAGGCGGGCAAGAATGTGAATAAAGGCCGGAT
AAAACTTGTGCTTATTTTTCTTTACGGTCTTTAAAAAGGCCGTAATATCCAGCTGAACGGTCTGGTT
ATAGGTACATTGAGCAACTGACTGAAATGCCTCAAAATGTTCTTTACGATGCCATTGGGATATATC
AACGGTGGTATATCCAGTGATTTTTTTCTCCATTTTAGCTTCCTTAGCTCCTGAAAATCTCGATAAC
TCAAAAAATACGCCCGGTAGTGATCTTATTTCATTATGGTGAAAGTTGGAACCTCTTACGTGCCGA
TCAACGTCTCATTTTCGCCAAAAGTTGGCCCAGGGCTTCCCGGTATCAACAGGGACACCAGGATTT
ATTTATTCTGCGAAGTGATCTTCCGTCACAGGTATTTATTCGCGATAAGCTCATGGAGCGGCGTAA
CCGTCGCACAGGAAGGACAGAGAAAGCGCGGATCTGGGAAGTGACGGACAGAACGGTCAGGACC
TGGATTGGGGAGGCGGTTGCCGCCGCTGCTGCTGACGGTGTGACGTTCTCTGTTCCGGTCACACCA
CATACGTTCCGCCATTCCTATGCGATGCACATGCTGTATGCCGGTATACCGCTGAAAGTTCTGCAA
AGCCTGATGGGACATAAGTCCATCAGTTCAACGGAAGTCTACACGAAGGTTTTTGCGCTGGATGTG
GCTGCCCGGCACCGGGTGCAGTTTGCGATGCCGGAGTCTGATGCGGTTGCGATGCTGAAACAATTA
TCCTGAGAATAAATGCCTTGGCCTTTATATGGAAATGTGGAACTGAGTGGATATGCTGTTTTTGTCT
GTTAAACAGAGAAGCTGGCTGTTATCCACTGAGAAGCGAACGAAACAGTCGGGAAAATCTCCCAT
TATCGTAGAGATCCGCATTATTAATCTCAGGAGCCTGTGTAGCGTTTATAGGAAGTAGTGTTCTGT
CATGATGCCTGCAAGCGGTAACGAAAACGATTTGAATATGCCTTCAGGAACAATAGAAATCTTCG
TGCGGTGTTACGTTGAAGTGGAGCGGATTATGTCAGCAATGGACAGAACAACCTAATGAACACAG
AACCATGATGTGGTCTGTCCTTTTACAGCCAGTAGTGCTCGCCGCAGTCGAGCGACAGGGCGAAGC
CCTCGAGTGAGCGAGGAAGCACCAGGGAACAGCACTTATATATTCTGCTTACACACGATGCCTGA
AAAAACTTCCCTTGGGGTTATCCACTTATCCACGGGGATATTTTTATAATTATTTTTTTTATAGTTTT
TAGATCTTCTTTTTTAGAGCGCCTTGTAGGCCTTTATCCATGCTGGTTCTAGAGAAGGTGTTGTGAC
AAATTGCCCTTTCAGTGTGACAAATCACCCTCAAATGACAGTCCTGTCTGTGACAAATTGCCCTTA
ACCCTGTGACAAATTGCCCTCAGAAGAAGCTGTTTTTTCACAAAGTTATCCCTGCTTATTGACTCTT
TTTTATTTAGTGTGACAATCTAAAAACTTGTCACACTTCACATGGATCTGTCATGGCGGAAACAGC
GGTTATCAATCACAAGAAACGTAAAAATAGCCCGCGAATCGTCCAGTCAAACGACCTCACTGAGG
CGGCATATAGTCTCTCCCGGGATCAAAAACGTATGCTGTATCTGTTCGTTGACCAGATCAGAAAAT
CTGATGGCACCCTACAGGAACATGACGGTATCTGCGAGATCCATGTTGCTAAATATGCTGAAATAT
TCGGATTGACCTCTGCGGAAGCCAGTAAGGATATACGGCAGGCATTGAAGAGTTTCGCGGGGAAG
GAAGTGGTTTTTTATCGCCCTGAAGAGGATGCCGGCGATGAAAAAGGCTATGAATCTTTTCCTTGG
TTTATCAAACGTGCGCACAGTCCATCCAGAGGGCTTTACAGTGTACATATCAACCCATATCTCATT
CCCTTCTTTATCGGGTTACAGAACCGGTTTACGCAGTTTCGGCTTAGTGAAACAAAAGAAATCACC
AATCCGTATGCCATGCGTTTATACGAATCCCTGTGTCAGTATCGTAAGCCGGATGGCTCAGGCATC
GTCTCTCTGAAAATCGACTGGATCATAGAGCGTTACCAGCTGCCTCAAAGTTACCAGCGTATGCCT
GACTTCCGCCGCCGCTTCCTGCAGGTCTGTGTTAATGAGATCAACAGCAGAACTCCAATGCGCCTC
TCATACATTGAGAAAAAGAAAGGCCGCCAGACGACTCATATCGTATTTTCCTTCCGCGATATCACT
TCCATGACGACAGGATAGTCTGAGGGTTATCTGTCACAGATTTGAGGGTGGTTCGTCACATTTGTT
CTGACCTACTGAGGGTAATTTGTCACAGTTTTGCTGTTTCCTTCAGCCTGCATGGATTTTCTCATAC
TTTTTGAACTGTAATTTTTAAGGAAGCCAAATTTGAGGGCAGTTTGTCACAGTTGATTTCCTTCTCT
TTCCCTTCGTCATGTGACCTGATATCGGGGGTTAGTTCGTCATCATTGATGAGGGTTGATTATCACA
GTTTATTACTCTGAATTGGCTATCCGCGTGTGTACCTCTACCTGGAGTTTTTCCCACGGTGGATATT
TCTTCTTGCGCTGAGCGTAAGAGCTATCTGACAGAACAGTTCTTCTTTGCTTCCTCGCCAGTTCGCT
CGCTATGCTCGGTTACACGGCTGCGGCGAGCGCTAGTGATAATAAGTGACTGAGGTATGTGCTCTT
CTTATCTCCTTTTGTAGTGTTGCTCTTATTTTAAACAACTTTGCGGTTTTTTGATGACTTTGCGATTT
TGTTGTTGCTTTGCAGTAAATTGCAAGATTTAATAAAAAAACGCAAAGCAATGATTAAAGGATGTT
CAGAATGAAACTCATGGAAACACTTAACCAGTGCATAAACGCTGGTCATGAAATGACGAAGGCTA
TCGCCATTGCACAGTTTAATGATGACAGCCCGGAAGCGAGGAAAATAACCCGGCGCTGGAGAATA
GGTGAAGCAGCGGATTTAGTTGGGGTTTCTTCTCAGGCTATCAGAGATGCCGAGAAAGCAGGGCG
ACTACCGCACCCGGATATGGAAATTCGAGGACGGGTTGAGCAACGTGTTGGTTATACAATTGAAC
AAATTAATCATATGCGTGATGTGTTTGGTACGCGATTGCGACGTGCTGAAGACGTATTTCCACCGG
TGATCGGGGTTGCTGCCCATAAAGGTGGCGTTTACAAAACCTCAGTTTCTGTTCATCTTGCTCAGG
ATCTGGCTCTGAAGGGGCTACGTGTTTTGCTCGTGGAAGGTAACGACCCCCAGGGAACAGCCTCA
ATGTATCACGGATGGGTACCAGATCTTCATATTCATGCAGAAGACACTCTCCTGCCTTTCTATCTTG
GGGAAAAGGACGATGTCACTTATGCAATAAAGCCCACTTGCTGGCCGGGGCTTGACATTATTCCTT
CCTGTCTGGCTCTGCACCGTATTGAAACTGAGTTAATGGGCAAATTTGATGAAGGTAAACTGCCCA
CCGATCCACACCTGATGCTCCGACTGGCCATTGAAACTGTTGCTCATGACTATGATGTCATAGTTA
TTGACAGCGCGCCTAACCTGGGTATCGGCACGATTAATGTCGTATGTGCTGCTGATGTGCTGATTG
TTCCCACGCCTGCTGAGTTGTTTGACTACACCTCCGCACTGCAGTTTTTCGATATGCTTCGTGATCT
GCTCAAGAACGTTGATCTTAAAGGGTTCGAGCCTGATGTACGTATTTTGCTTACCAAATACAGCAA
TAGTAATGGCTCTCAGTCCCCGTGGATGGAGGAGCAAATTCGGGATGCCTGGGGAAGCATGGTTC
TAAAAAATGTTGTACGTGAAACGGATGAAGTTGGTAAAGGTCAGATCCGGATGAGAACTGTTTTT
GAACAGGCCATTGATCAACGCTCTTCAACTGGTGCCTGGAGAAATGCTCTTTCTATTTGGGAACCT
GTCTGCAATGAAATTTTCGATCGTCTGATTAAACCACGCTGGGAGATTAGATAATGAAGCGTGCGC
CTGTTATTCCAAAACATACGCTCAATACTCAACCGGTTGAAGATACTTCGTTATCGACACCAGCTG
CCCCGATGGTGGATTCGTTAATTGCGCGCGTAGGAGTAATGGCTCGCGGTAATGCCATTACTTTGC
CTGTATGTGGTCGGGATGTGAAGTTTACTCTTGAAGTGCTCCGGGGTGATAGTGTTGAGAAGACCT
CTCGGGTATGGTCAGGTAATGAACGTGACCAGGAGCTGCTTACTGAGGACGCACTGGATGATCTC
ATCCCTTCTTTTCTACTGACTGGTCAACAGACACCGGCGTTCGGTCGAAGAGTATCTGGTGTCATA
GAAATTGCCGATGGGAGTCGCCGTCGTAAAGCTGCTGCACTTACCGAAAGTGATTATCGTGTTCTG
GTTGGCGAGCTGGATGATGAGCAGATGGCTGCATTATCCAGATTGGGTAACGATTATCGCCCAAC
AAGTGCTTATGAACGTGGTCAGCGTTATGCAAGCCGATTGCAGAATGAATTTGCTGGAAATATTTC
TGCGCTGGCTGATGCGGAAAATATTTCACGTAAGATTATTACCCGCTGTATCAACACCGCCAAATT
GCCTAAATCAGTTGTTGCTCTTTTTTCTCACCCCGGTGAACTATCTGCCCGGTCAGGTGATGCACTT
CAAAAAGCCTTTACAGATAAAGAGGAATTACTTAAGCAGCAGGCATCTAACCTTCATGAGCAGAA
AAAAGCTGGGGTGATATTTGAAGCTGAAGAAGTTATCACTCTTTTAACTTCTGTGCTTAAAACGTC
ATCTGCATCAAGAACTAGTTTAAGCTCACGACATCAGTTTGCTCCTGGAGCGACAGTATTGTATAA
GGGCGATAAAATGGTGCTTAACCTGGACAGGTCTCGTGTTCCAACTGAGTGTATAGAGAAAATTG
AGGCCATTCTTAAGGAACTTGAAAAGCCAGCACCCTGATGCGACCACGTTTTAGTCTACGTTTATC
TGTCTTTACTTAATGTCCTTTGTTACAGGCCAGAAAGCATAACTGGCCTGAATATTCTCTCTGGGCC
CACTGTTCCACTTGTATCGTCGGTCTGATAATCAGACTGGGACCACGGTCCCACTCGTATCGTCGG
TCTGATTATTAGTCTGGGACCACGGTCCCACTCGTATCGTCGGTCTGATTATTAGTCTGGGACCACG
GTCCCACTCGTATCGTCGGTCTGATAATCAGACTGGGACCACGGTCCCACTCGTATCGTCGGTCTG
ATTATTAGTCTGGGACCATGGTCCCACTCGTATCGTCGGTCTGATTATTAGTCTGGGACCACGGTCC
CACTCGTATCGTCGGTCTGATTATTAGTCTGGAACCACGGTCCCACTCGTATCGTCGGTCTGATTAT
TAGTCTGGGACCACGGTCCCACTCGTATCGTCGGTCTGATTATTAGTCTGGGACCACGATCCCACT
CGTGTTGTCGGTCTGATTATCGGTCTGGGACCACGGTCCCACTTGTATTGTCGATCAGACTATCAGC
GTGAGACTACGATTCCATCAATGCCTGTCAAGGGCAAGTATTGACATGTCGTCGTAACCTGTAGAA
CGGAGTAACCTCGGTGTGCGGTTGTATGCCTGCTGTGGATTGCTGCTGTGTCCTGCTTATCCACAAC
ATTTTGCGCACGGTTATGTGGACAAAATACCTGGTTACCCAGGCCGTGCCGGCACGTTAACCGGGC
ACATTTCCCCGAAAAGTGCCACCTGACGTCTAAGAAACCATTATTATCATGACATTAACCTATAAA
AATAGGCGTATCACGAGGCCCTTTCGTCTTCAAGAATTGGATCCGAATTCCCGGGAGAGCTCGATA
TCGCATGCGGATTTAAATTAATTAA
* C1E
(SEQ ID NO: 86)
CATCATCAATAATATACCTTATTTTGGATTGAAGCCAATATGATAATGAGGGGGTGGAGTTTGTGA
CGTGGCGCGGGGCGTGGGAACGGGGCGGGTGACGTAGTAGTGTGGCGGAAGTGTGATGTTGCAAG
TGTGGCGGAACACATGTAAGCGACGGATGTGGCAAAAGTGACGTTTTTGGTGTGCGCCGGTGTAC
ACAGGAAGTGACAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAATTTTGTGTTACT
CATAGCGCGTAATATTTGTCTAGGGCCGCGGGGACTTTGACCGTTTACGTGGAGACTCGCCCAGGT
GTTTTTCTCAGGTGTTTTCCGCGTTCCGGGTCAAAGTTGGCGTTTTATTATTATAGTCAGTCGAAGC
TTGGATCCGGTACCTCTAGAATTCTCGAGCGGCCGCTAGCGACATCGGATCTCCCGATCCCCTATG
GTGCACTCTCAGTACAATCTGCTCTGATGCCGCATAGTTAAGCCAGTATCTGCTCCCTGCTTGTGTG
TTGGAGGTCGCTGAGTAGTGCGCGAGCAAAATTTAAGCTACAACAAGGCAAGGCTTGACCGACAA
TTGCATGAAGAATCTGCTTAGGGTTAGGCGTTTTGCGCTGCTTCGCGATGTACGGGCCAGATATAC
GCGTTGACATTGATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACG
TCAATGGGTGGACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCTCTG
GCTAACTAGAGAACCCACTGCTTACTGGCTTATCGAAATTAATACGACTCACTATAGGGAGACCCA
AGCTGGCTAGTTAAGCTATCAACAAGTTTGTACAAAAAAGCAGGCTTTAAAGGAACCAATTCAGT
CGACTGGATCCGGTACCACCATGTTCCTGAACTGCTGCCCAGGTTGCTGTATGGAGCCTGAATTCA
CCATGGTGAGCAAGGGCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACCCT
GACCTGGGGCGTGCAGTGCTTCGCCCGCTACCCCGACCACATGAAGCAGCACGACTTCTTCAAGTC
CGCCATGCCCGAAGGCTACGTCCAGGAGCGCACCATCTTCTTCAAGGACGACGGCAACTACAAGA
CCCGCGCCGAGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACGCCATCAGCGACAACGTCTA
TATCACCGCCGACAAGCAGAAGAACGGCATCAAGGCCAACTTCAAGATCCGCCACAACATCGAGG
ACGGCAGCGTGCAGCTCGCCGACCACTACCAGCAGAACACCCCCATCGGCGACGGCCCCGTGCTG
CTGCCCGACAACCACTACCTGAGCACCCAGTCCAAGCTGAGCAAAGACCCCAACGAGAAGCGCGA
TCACATGGTCCTGCTGGAGTTCGTGACCGCCGCCGGGATCACTCTCGGCATGGACGAGCTGTACAA
GGTCGACTATCCGTACGACGTACCAGACTACGCATAACCGCGGCCGCACTCGAGATATCTAGACC
CAGCTTTCTTGTACAAAGTGGTTGATCTAGAGGGCCCGCGGTTCGAAGGTAAGCCTATCCCTAACC
CTCTCCTCGGTCTCGATTCTACGCGTACCGGTTAGTAATGAGTTTAAACGGGGGAGGCTAACTGAA
ACACGGAAGGAGACAATACCGGAAGGAACCCGCGCTATGACGGCAATAAAAAGACAGAATAAAA
CGCACGGGTGTTGGGTCGTTTGTTCATAAACGCGGGGTTCGGTCCCAGGGCTGGCACTCTGTCGAT
ACCCCACCGAGACCCCATTGGGGCCAATACGCCCGCGTTTCTTCCTTTTCCCCACCCCACCCCCCA
AGTTCGGGTGAAGGCCCAGGGCTCGCAGCCAACGTCGGGGCGGCAGGCCCTGCCATAGCAGATCC
GATTCGACAGATCACTGAAATGTGTGGGCGTGGCTTAAGGGTGGGAAAGAATATATAAGGTGGGG
GTCTTATGTAGTTTTGTATCTGTTTTGCAGCAGCCGCCGCCGCCATGAGCACCAACTCGTTTGATGG
AAGCATTGTGAGCTCATATTTGACAACGCGCATGCCCCCATGGGCCGGGGTGCGTCAGAATGTGAT
GGGCTCCAGCATTGATGGTCGCCCCGTCCTGCCCGCAAACTCTACTACCTTGACCTACGAGACCGT
GTCTGGAACGCCGTTGGAGACTGCAGCCTCCGCCGCCGCTTCAGCCGCTGCAGCCACCGCCCGCGG
GATTGTGACTGACTTTGCTTTCCTGAGCCCGCTTGCAAGCAGTGCAGCTTCCCGTTCATCCGCCCGC
GATGACAAGTTGACGGCTCTTTTGGCACAATTGGATTCTTTGACCCGGGAACTTAATGTCGTTTCTC
AGCAGCTGTTGGATCTGCGCCAGCAGGTTTCTGCCCTGAAGGCTTCCTCCCCTCCCAATGCGGTTT
AAAACATAAATAAAAAACCAGACTCTGTTTGGATTTGGATCAAGCAAGTGTCTTGCTGTCTTTATT
TAGGGGTTTTGCGCGCGCGGTAGGCCCGGGACCAGCGGTCTCGGTCGTTGAGGGTCCTGTGTATTT
TTTCCAGGACGTGGTAAAGGTGACTCTGGATGTTCAGATACATGGGCATAAGCCCGTCTCTGGGGT
GGAGGTAGCACCACTGCAGAGCTTCATGCTGCGGGGTGGTGTTGTAGATGATCCAGTCGTAGCAG
GAGCGCTGGGCGTGGTGCCTAAAAATGTCTTTCAGTAGCAAGCTGATTGCCAGGGGCAGGCCCTT
GGTGTAAGTGTTTACAAAGCGGTTAAGCTGGGATGGGTGCATACGTGGGGATATGAGATGCATCT
TGGACTGTATTTTTAGGTTGGCTATGTTCCCAGCCATATCCCTCCGGGGATTCATGTTGTGCAGAAC
CACCAGCACAGTGTATCCGGTGCACTTGGGAAATTTGTCATGTAGCTTAGAAGGAAATGCGTGGA
AGAACTTGGAGACGCCCTTGTGACCTCCAAGATTTTCCATGCATTCGTCCATAATGATGGCAATGG
GCCCACGGGCGGCGGCCTGGGCGAAGATATTTCTGGGATCACTAACGTCATAGTTGTGTTCCAGGA
TGAGATCGTCATAGGCCATTTTTACAAAGCGCGGGCGGAGGGTGCCAGACTGCGGTATAATGGTT
CCATCCGGCCCAGGGGCGTAGTTACCCTCACAGATTTGCATTTCCCACGCTTTGAGTTCAGATGGG
GGGATCATGTCTACCTGCGGGGCGATGAAGAAAACGGTTTCCGGGGTAGGGGAGATCAGCTGGGA
AGAAAGCAGGTTCCTGAGCAGCTGCGACTTACCGCAGCCGGTGGGCCCGTAAATCACACCTATTA
CCGGCTGCAACTGGTAGTTAAGAGAGCTGCAGCTGCCGTCATCCCTGAGCAGGGGGGCCACTTCG
TTAAGCATGTCCCTGACTCGCATGTTTTCCCTGACCAAATCCGCCAGAAGGCGCTCGCCGCCCAGC
GATAGCAGTTCTTGCAAGGAAGCAAAGTTTTTCAACGGTTTGAGACCGTCCGCCGTAGGCATGCTT
TTGAGCGTTTGACCAAGCAGTTCCAGGCGGTCCCACAGCTCGGTCACCTGCTCTACGGCATCTCGA
TCCAGCATATCTCCTCGTTTCGCGGGTTGGGGCGGCTTTCGCTGTACGGCAGTAGTCGGTGCTCGTC
CAGACGGGCCAGGGTCATGTCTTTCCACGGGCGCAGGGTCCTCGTCAGCGTAGTCTGGGTCACGGT
GAAGGGGTGCGCTCCGGGCTGCGCGCTGGCCAGGGTGCGCTTGAGGCTGGTCCTGCTGGTGCTGA
AGCGCTGCCGGTCTTCGCCCTGCGCGTCGGCCAGGTAGCATTTGACCATGGTGTCATAGTCCAGCC
CCTCCGCGGCGTGGCCCTTGGCGCGCAGCTTGCCCTTGGAGGAGGCGCCGCACGAGGGGCAGTGC
AGACTTTTGAGGGCGTAGAGCTTGGGCGCGAGAAATACCGATTCCGGGGAGTAGGCATCCGCGCC
GCAGGCCCCGCAGACGGTCTCGCATTCCACGAGCCAGGTGAGCTCTGGCCGTTCGGGGTCAAAAA
CCAGGTTTCCCCCATGCTTTTTGATGCGTTTCTTACCTCTGGTTTCCATGAGCCGGTGTCCACGCTC
GGTGACGAAAAGGCTGTCCGTGTCCCCGTATACAGACTTGAGAGGCCTGTCCTCGAGCGGTGTTCC
GCGGTCCTCCTCGTATAGAAACTCGGACCACTCTGAGACAAAGGCTCGCGTCCAGGCCAGCACGA
AGGAGGCTAAGTGGGAGGGGTAGCGGTCGTTGTCCACTAGGGGGTCCACTCGCTCCAGGGTGTGA
AGACACATGTCGCCCTCTTCGGCATCAAGGAAGGTGATTGGTTTGTAGGTGTAGGCCACGTGACCG
GGTGTTCCTGAAGGGGGGCTATAAAAGGGGGTGGGGGCGCGTTCGTCCTCACTCTCTTCCGCATCG
CTGTCTGCGAGGGCCAGCTGTTGGGGTGAGTACTCCCTCTGAAAAGCGGGCATGACTTCTGCGCTA
AGATTGTCAGTTTCCAAAAACGAGGAGGATTTGATATTCACCTGGCCCGCGGTGATGCCTTTGAGG
GTGGCCGCATCCATCTGGTCAGAAAAGACAATCTTTTTGTTGTCAAGCTTGGTGGCAAACGACCCG
TAGAGGGCGTTGGACAGCAACTTGGCGATGGAGCGCAGGGTTTGGTTTTTGTCGCGATCGGCGCG
CTCCTTGGCCGCGATGTTTAGCTGCACGTATTCGCGCGCAACGCACCGCCATTCGGGAAAGACGGT
GGTGCGCTCGTCGGGCACCAGGTGCACGCGCCAACCGCGGTTGTGCAGGGTGACAAGGTCAACGC
TGGTGGCTACCTCTCCGCGTAGGCGCTCGTTGGTCCAGCAGAGGCGGCCGCCCTTGCGCGAGCAGA
ATGGCGGTAGGGGGTCTAGCTGCGTCTCGTCCGGGGGGTCTGCGTCCACGGTAAAGACCCCGGGC
AGCAGGCGCGCGTCGAAGTAGTCTATCTTGCATCCTTGCAAGTCTAGCGCCTGCTGCCATGCGCGG
GCGGCAAGCGCGCGCTCGTATGGGTTGAGTGGGGGACCCCATGGCATGGGGTGGGTGAGCGCGGA
GGCGTACATGCCGCAAATGTCGTAAACGTAGAGGGGCTCTCTGAGTATTCCAAGATATGTAGGGT
AGCATCTTCCACCGCGGATGCTGGCGCGCACGTAATCGTATAGTTCGTGCGAGGGAGCGAGGAGG
TCGGGACCGAGGTTGCTACGGGCGGGCTGCTCTGCTCGGAAGACTATCTGCCTGAAGATGGCATGT
GAGTTGGATGATATGGTTGGACGCTGGAAGACGTTGAAGCTGGCGTCTGTGAGACCTACCGCGTC
ACGCACGAAGGAGGCGTAGGAGTCGCGCAGCTTGTTGACCAGCTCGGCGGTGACCTGCACGTCTA
GGGCGCAGTAGTCCAGGGTTTCCTTGATGATGTCATACTTATCCTGTCCCTTTTTTTTCCACAGCTC
GCGGTTGAGGACAAACTCTTCGCGGTCTTTCCAGTACTCTTGGATCGGAAACCCGTCGGCCTCCGA
ACGGTAAGAGCCTAGCATGTAGAACTGGTTGACGGCCTGGTAGGCGCAGCATCCCTTTTCTACGGG
TAGCGCGTATGCCTGCGCGGCCTTCCGGAGCGAGGTGTGGGTGAGCGCAAAGGTGTCCCTGACCA
TGACCAGCATGAAGGGCACGAGCTGCTTCCCAAAGGCCCCCATCCAAGTATAGGTCTCTACATCGT
AGGTGACAAAGAGACGCTCGGTGCGAGGATGCGAGCCGATCGGGAAGAACTGGATCTCCCGCCAC
CAATTGGAGGAGTGGCTATTGATGTGGTGAAAGTAGAAGTCCCTGCGACGGGCCGAACACTCGTG
CTGGCTTTTGTAAAAACGTGCGCAGTACTGGCAGCGGTGCACGGGCTGTACATCCTGCACGAGGTT
GACCTGACGACCGCGCACAAGGAAGCAGAGTGGGAATTTGAGCCCCTCGCCTGGCGGGTTTGGCT
GGTGGTCTTCTACTTCGGCTGCTTGTCCTTGACCGTCTGGCTGCTCGAGGGGAGTTACGGTGGATC
GGACCACCACGCCGCGCGAGCCCAAAGTCCAGATGTCCGCGCGCGGCGGTCGGAGCTTGATGACA
ACATCGCGCAGATGGGAGCTGTCCATGGTCTGGAGCTCCCGCGGCGTCAGGTCAGGCGGGAGCTC
CTGCAGGTTTACCTCGCATAGACGGGTCAGGGCGCGGGCTAGATCCAGGTGATACCTAATTTCCAG
GGGCTGGTTGGTGGCGGCGTCGATGGCTTGCAAGAGGCCGCATCCCCGCGGCGCGACTACGGTAC
CGCGCGGCGGGCGGTGGGCCGCGGGGGTGTCCTTGGATGATGCATCTAAAAGCGGTGACGCGGGC
GAGCCCCCGGAGGTAGGGGGGGCTCCGGACCCGCCGGGAGAGGGGGCAGGGGCACGTCGGCGCC
GCGCGCGGGCAGGAGCTGGTGCTGCGCGCGTAGGTTGCTGGCGAACGCGACGACGCGGCGGTTGA
TCTCCTGAATCTGGCGCCTCTGCGTGAAGACGACGGGCCCGGTGAGCTTGAACCTGAAAGAGAGT
TCGACAGAATCAATTTCGGTGTCGTTGACGGCGGCCTGGCGCAAAATCTCCTGCACGTCTCCTGAG
TTGTCTTGATAGGCGATCTCGGCCATGAACTGCTCGATCTCTTCCTCCTGGAGATCTCCGCGTCCGG
CTCGCTCCACGGTGGCGGCGAGGTCGTTGGAAATGCGGGCCATGAGCTGCGAGAAGGCGTTGAGG
CCTCCCTCGTTCCAGACGCGGCTGTAGACCACGCCCCCTTCGGCATCGCGGGCGCGCATGACCACC
TGCGCGAGATTGAGCTCCACGTGCCGGGCGAAGACGGCGTAGTTTCGCAGGCGCTGAAAGAGGTA
GTTGAGGGTGGTGGCGGTGTGTTCTGCCACGAAGAAGTACATAACCCAGCGTCGCAACGTGGATT
CGTTGATATCCCCCAAGGCCTCAAGGCGCTCCATGGCCTCGTAGAAGTCCACGGCGAAGTTGAAA
AACTGGGAGTTGCGCGCCGACACGGTTAACTCCTCCTCCAGAAGACGGATGAGCTCGGCGACAGT
GTCGCGCACCTCGCGCTCAAAGGCTACAGGGGCCTCTTCTTCTTCTTCAATCTCCTCTTCCATAAGG
GCCTCCCCTTCTTCTTCTTCTGGCGGCGGTGGGGGAGGGGGGACACGGCGGCGACGACGGCGCAC
CGGGAGGCGGTCGACAAAGCGCTCGATCATCTCCCCGCGGCGACGGCGCATGGTCTCGGTGACGG
CGCGGCCGTTCTCGCGGGGGCGCAGTTGGAAGACGCCGCCCGTCATGTCCCGGTTATGGGTTGGCG
GGGGGCTGCCATGCGGCAGGGATACGGCGCTAACGATGCATCTCAACAATTGTTGTGTAGGTACT
CCGCCGCCGAGGGACCTGAGCGAGTCCGCATCGACCGGATCGGAAAACCTCTCGAGAAAGGCGTC
TAACCAGTCACAGTCGCAAGGTAGGCTGAGCACCGTGGCGGGCGGCAGCGGGCGGCGGTCGGGGT
TGTTTCTGGCGGAGGTGCTGCTGATGATGTAATTAAAGTAGGCGGTCTTGAGACGGCGGATGGTCG
ACAGAAGCACCATGTCCTTGGGTCCGGCCTGCTGAATGCGCAGGCGGTCGGCCATGCCCCAGGCTT
CGTTTTGACATCGGCGCAGGTCTTTGTAGTAGTCTTGCATGAGCCTTTCTACCGGCACTTCTTCTTC
TCCTTCCTCTTGTCCTGCATCTCTTGCATCTATCGCTGCGGCGGCGGCGGAGTTTGGCCGTAGGTGG
CGCCCTCTTCCTCCCATGCGTGTGACCCCGAAGCCCCTCATCGGCTGAAGCAGGGCTAGGTCGGCG
ACAACGCGCTCGGCTAATATGGCCTGCTGCACCTGCGTGAGGGTAGACTGGAAGTCATCCATGTCC
ACAAAGCGGTGGTATGCGCCCGTGTTGATGGTGTAAGTGCAGTTGGCCATAACGGACCAGTTAAC
GGTCTGGTGACCCGGCTGCGAGAGCTCGGTGTACCTGAGACGCGAGTAAGCCCTCGAGTCAAATA
CGTAGTCGTTGCAAGTCCGCACCAGGTACTGGTATCCCACCAAAAAGTGCGGCGGCGGCTGGCGG
TAGAGGGGCCAGCGTAGGGTGGCCGGGGCTCCGGGGGCGAGATCTTCCAACATAAGGCGATGATA
TCCGTAGATGTACCTGGACATCCAGGTGATGCCGGCGGCGGTGGTGGAGGCGCGCGGAAAGTCGC
GGACGCGGTTCCAGATGTTGCGCAGCGGCAAAAAGTGCTCCATGGTCGGGACGCTCTGGCCGGTC
AGGCGCGCGCAATCGTTGACGCTCTAGACCGTGCAAAAGGAGAGCCTGTAAGCGGGCACTCTTCC
GTGGTCTGGTGGATAAATTCGCAAGGGTATCATGGCGGACGACCGGGGTTCGAGCCCCGTATCCG
GCCGTCCGCCGTGATCCATGCGGTTACCGCCCGCGTGTCGAACCCAGGTGTGCGACGTCAGACAAC
GGGGGAGTGCTCCTTTTGGCTTCCTTCCAGGCGCGGCGGCTGCTGCGCTAGCTTTTTTGGCCACTGG
CCGCGCGCAGCGTAAGCGGTTAGGCTGGAAAGCGAAAGCATTAAGTGGCTCGCTCCCTGTAGCCG
GAGGGTTATTTTCCAAGGGTTGAGTCGCGGGACCCCCGGTTCGAGTCTCGGACCGGCCGGACTGCG
GCGAACGGGGGTTTGCCTCCCCGTCATGCAAGACCCCGCTTGCAAATTCCTCCGGAAACAGGGAC
GAGCCCCTTTTTTGCTTTTCCCAGATGCATCCGGTGCTGCGGCAGATGCGCCCCCCTCCTCAGCAGC
GGCAAGAGCAAGAGCAGCGGCAGACATGCAGGGCACCCTCCCCTCCTCCTACCGCGTCAGGAGGG
GCGACATCCGCGGTTGACGCGGCAGCAGATGGTGATTACGAACCCCCGCGGCGCCGGGCCCGGCA
CTACCTGGACTTGGAGGAGGGCGAGGGCCTGGCGCGGCTAGGAGCGCCCTCTCCTGAGCGGCACC
CAAGGGTGCAGCTGAAGCGTGATACGCGTGAGGCGTACGTGCCGCGGCAGAACCTGTTTCGCGAC
CGCGAGGGAGAGGAGCCCGAGGAGATGCGGGATCGAAAGTTCCACGCAGGGCGCGAGCTGCGGC
ATGGCCTGAATCGCGAGCGGTTGCTGCGCGAGGAGGACTTTGAGCCCGACGCGCGAACCGGGATT
AGTCCCGCGCGCGCACACGTGGCGGCCGCCGACCTGGTAACCGCATACGAGCAGACGGTGAACCA
GGAGATTAACTTTCAAAAAAGCTTTAACAACCACGTGCGTACGCTTGTGGCGCGCGAGGAGGTGG
CTATAGGACTGATGCATCTGTGGGACTTTGTAAGCGCGCTGGAGCAAAACCCAAATAGCAAGCCG
CTCATGGCGCAGCTGTTCCTTATAGTGCAGCACAGCAGGGACAACGAGGCATTCAGGGATGCGCT
GCTAAACATAGTAGAGCCCGAGGGCCGCTGGCTGCTCGATTTGATAAACATCCTGCAGAGCATAG
TGGTGCAGGAGCGCAGCTTGAGCCTGGCTGACAAGGTGGCCGCCATCAACTATTCCATGCTTAGCC
TGGGCAAGTTTTACGCCCGCAAGATATACCATACCCCTTACGTTCCCATAGACAAGGAGGTAAAG
ATCGAGGGGTTCTACATGCGCATGGCGCTGAAGGTGCTTACCTTGAGCGACGACCTGGGCGTTTAT
CGCAACGAGCGCATCCACAAGGCCGTGAGCGTGAGCCGGCGGCGCGAGCTCAGCGACCGCGAGCT
GATGCACAGCCTGCAAAGGGCCCTGGCTGGCACGGGCAGCGGCGATAGAGAGGCCGAGTCCTACT
TTGACGCGGGCGCTGACCTGCGCTGGGCCCCAAGCCGACGCGCCCTGGAGGCAGCTGGGGCCGGA
CCTGGGCTGGCGGTGGCACCCGCGCGCGCTGGCAACGTCGGCGGCGTGGAGGAATATGACGAGGA
CGATGAGTACGAGCCAGAGGACGGCGAGTACTAAGCGGTGATGTTTCTGATCAGATGATGCAAGA
CGCAACGGACCCGGCGGTGCGGGCGGCGCTGCAGAGCCAGCCGTCCGGCCTTAACTCCACGGACG
ACTGGCGCCAGGTCATGGACCGCATCATGTCGCTGACTGCGCGCAATCCTGACGCGTTCCGGCAGC
AGCCGCAGGCCAACCGGCTCTCCGCAATTCTGGAAGCGGTGGTCCCGGCGCGCGCAAACCCCACG
CACGAGAAGGTGCTGGCGATCGTAAACGCGCTGGCCGAAAACAGGGCCATCCGGCCCGACGAGG
CCGGCCTGGTCTACGACGCGCTGCTTCAGCGCGTGGCTCGTTACAACAGCGGCAACGTGCAGACC
AACCTGGACCGGCTGGTGGGGGATGTGCGCGAGGCCGTGGCGCAGCGTGAGCGCGCGCAGCAGC
AGGGCAACCTGGGCTCCATGGTTGCACTAAACGCCTTCCTGAGTACACAGCCCGCCAACGTGCCGC
GGGGACAGGAGGACTACACCAACTTTGTGAGCGCACTGCGGCTAATGGTGACTGAGACACCGCAA
AGTGAGGTGTACCAGTCTGGGCCAGACTATTTTTTCCAGACCAGTAGACAAGGCCTGCAGACCGTA
AACCTGAGCCAGGCTTTCAAAAACTTGCAGGGGCTGTGGGGGGTGCGGGCTCCCACAGGCGACCG
CGCGACCGTGTCTAGCTTGCTGACGCCCAACTCGCGCCTGTTGCTGCTGCTAATAGCGCCCTTCAC
GGACAGTGGCAGCGTGTCCCGGGACACATACCTAGGTCACTTGCTGACACTGTACCGCGAGGCCA
TAGGTCAGGCGCATGTGGACGAGCATACTTTCCAGGAGATTACAAGTGTCAGCCGCGCGCTGGGG
CAGGAGGACACGGGCAGCCTGGAGGCAACCCTAAACTACCTGCTGACCAACCGGCGGCAGAAGA
TCCCCTCGTTGCACAGTTTAAACAGCGAGGAGGAGCGCATTTTGCGCTACGTGCAGCAGAGCGTG
AGCCTTAACCTGATGCGCGACGGGGTAACGCCCAGCGTGGCGCTGGACATGACCGCGCGCAACAT
GGAACCGGGCATGTATGCCTCAAACCGGCCGTTTATCAACCGCCTAATGGACTACTTGCATCGCGC
GGCCGCCGTGAACCCCGAGTATTTCACCAATGCCATCTTGAACCCGCACTGGCTACCGCCCCCTGG
TTTCTACACCGGGGGATTCGAGGTGCCCGAGGGTAACGATGGATTCCTCTGGGACGACATAGACG
ACAGCGTGTTTTCCCCGCAACCGCAGACCCTGCTAGAGTTGCAACAGCGCGAGCAGGCAGAGGCG
GCGCTGCGAAAGGAAAGCTTCCGCAGGCCAAGCAGCTTGTCCGATCTAGGCGCTGCGGCCCCGCG
GTCAGATGCTAGTAGCCCATTTCCAAGCTTGATAGGGTCTCTTACCAGCACTCGCACCACCCGCCC
GCGCCTGCTGGGCGAGGAGGAGTACCTAAACAACTCGCTGCTGCAGCCGCAGCGCGAAAAAAACC
TGCCTCCGGCATTTCCCAACAACGGGATAGAGAGCCTAGTGGACAAGATGAGTAGATGGAAGACG
TACGCGCAGGAGCACAGGGACGTGCCAGGCCCGCGCCCGCCCACCCGTCGTCAAAGGCACGACCG
TCAGCGGGGTCTGGTGTGGGAGGACGATGACTCGGCAGACGACAGCAGCGTCCTGGATTTGGGAG
GGAGTGGCAACCCGTTTGCGCACCTTCGCCCCAGGCTGGGGAGAATGTTTTAAAAAAAAAAAAAG
CATGATGCAAAATAAAAAACTCACCAAGGCCATGGCACCGAGCGTTGGTTTTCTTGTATTCCCCTT
AGTATGCGGCGCGCGGCGATGTATGAGGAAGGTCCTCCTCCCTCCTACGAGAGTGTGGTGAGCGC
GGCGCCAGTGGCGGCGGCGCTGGGTTCTCCCTTCGATGCTCCCCTGGACCCGCCGTTTGTGCCTCC
GCGGTACCTGCGGCCTACCGGGGGGAGAAACAGCATCCGTTACTCTGAGTTGGCACCCCTATTCGA
CACCACCCGTGTGTACCTGGTGGACAACAAGTCAACGGATGTGGCATCCCTGAACTACCAGAACG
ACCACAGCAACTTTCTGACCACGGTCATTCAAAACAATGACTACAGCCCGGGGGAGGCAAGCACA
CAGACCATCAATCTTGACGACCGGTCGCACTGGGGCGGCGACCTGAAAACCATCCTGCATACCAA
CATGCCAAATGTGAACGAGTTCATGTTTACCAATAAGTTTAAGGCGCGGGTGATGGTGTCGCGCTT
GCCTACTAAGGACAATCAGGTGGAGCTGAAATACGAGTGGGTGGAGTTCACGCTGCCCGAGGGCA
ACTACTCCGAGACCATGACCATAGACCTTATGAACAACGCGATCGTGGAGCACTACTTGAAAGTG
GGCAGACAGAACGGGGTTCTGGAAAGCGACATCGGGGTAAAGTTTGACACCCGCAACTTCAGACT
GGGGTTTGACCCCGTCACTGGTCTTGTCATGCCTGGGGTATATACAAACGAAGCCTTCCATCCAGA
CATCATTTTGCTGCCAGGATGCGGGGTGGACTTCACCCACAGCCGCCTGAGCAACTTGTTGGGCAT
CCGCAAGCGGCAACCCTTCCAGGAGGGCTTTAGGATCACCTACGATGATCTGGAGGGTGGTAACA
TTCCCGCACTGTTGGATGTGGACGCCTACCAGGCGAGCTTGAAAGATGACACCGAACAGGGCGGG
GGTGGCGCAGGCGGCAGCAACAGCAGTGGCAGCGGCGCGGAAGAGAACTCCAACGCGGCAGCCG
CGGCAATGCAGCCGGTGGAGGACATGAACGATCATGCCATTCGCGGCGACACCTTTGCCACACGG
GCTGAGGAGAAGCGCGCTGAGGCCGAAGCAGCGGCCGAAGCTGCCGCCCCCGCTGCGCAACCCG
AGGTCGAGAAGCCTCAGAAGAAACCGGTGATCAAACCCCTGACAGAGGACAGCAAGAAACGCAG
TTACAACCTAATAAGCAATGACAGCACCTTCACCCAGTACCGCAGCTGGTACCTTGCATACAACTA
CGGCGACCCTCAGACCGGAATCCGCTCATGGACCCTGCTTTGCACTCCTGACGTAACCTGCGGCTC
GGAGCAGGTCTACTGGTCGTTGCCAGACATGATGCAAGACCCCGTGACCTTCCGCTCCACGCGCCA
GATCAGCAACTTTCCGGTGGTGGGCGCCGAGCTGTTGCCCGTGCACTCCAAGAGCTTCTACAACGA
CCAGGCCGTCTACTCCCAACTCATCCGCCAGTTTACCTCTCTGACCCACGTGTTCAATCGCTTTCCC
GAGAACCAGATTTTGGCGCGCCCGCCAGCCCCCACCATCACCACCGTCAGTGAAAACGTTCCTGCT
CTCACAGATCACGGGACGCTACCGCTGCGCAACAGCATCGGAGGAGTCCAGCGAGTGACCATTAC
TGACGCCAGACGCCGCACCTGCCCCTACGTTTACAAGGCCCTGGGCATAGTCTCGCCGCGCGTCCT
ATCGAGCCGCACTTTTTGAGCAAGCATGTCCATCCTTATATCGCCCAGCAATAACACAGGCTGGGG
CCTGCGCTTCCCAAGCAAGATGTTTGGCGGGGCCAAGAAGCGCTCCGACCAACACCCAGTGCGCG
TGCGCGGGCACTACCGCGCGCCCTGGGGCGCGCACAAACGCGGCCGCACTGGGCGCACCACCGTC
GATGACGCCATCGACGCGGTGGTGGAGGAGGCGCGCAACTACACGCCCACGCCGCCACCAGTGTC
CACAGTGGACGCGGCCATTCAGACCGTGGTGCGCGGAGCCCGGCGCTATGCTAAAATGAAGAGAC
GGCGGAGGCGCGTAGCACGTCGCCACCGCCGCCGACCCGGCACTGCCGCCCAACGCGCGGCGGCG
GCCCTGCTTAACCGCGCACGTCGCACCGGCCGACGGGCGGCCATGCGGGCCGCTCGAAGGCTGGC
CGCGGGTATTGTCACTGTGCCCCCCAGGTCCAGGCGACGAGCGGCCGCCGCAGCAGCCGCGGCCA
TTAGTGCTATGACTCAGGGTCGCAGGGGCAACGTGTATTGGGTGCGCGACTCGGTTAGCGGCCTGC
GCGTGCCCGTGCGCACCCGCCCCCCGCGCAACTAGATTGCAAGAAAAAACTACTTAGACTCGTACT
GTTGTATGTATCCAGCGGCGGCGGCGCGCAACGAAGCTATGTCCAAGCGCAAAATCAAAGAAGAG
ATGCTCCAGGTCATCGCGCCGGAGATCTATGGCCCCCCGAAGAAGGAAGAGCAGGATTACAAGCC
CCGAAAGCTAAAGCGGGTCAAAAAGAAAAAGAAAGATGATGATGATGAACTTGACGACGAGGTG
GAACTGCTGCACGCTACCGCGCCCAGGCGACGGGTACAGTGGAAAGGTCGACGCGTAAAACGTGT
TTTGCGACCCGGCACCACCGTAGTCTTTACGCCCGGTGAGCGCTCCACCCGCACCTACAAGCGCGT
GTATGATGAGGTGTACGGCGACGAGGACCTGCTTGAGCAGGCCAACGAGCGCCTCGGGGAGTTTG
CCTACGGAAAGCGGCATAAGGACATGCTGGCGTTGCCGCTGGACGAGGGCAACCCAACACCTAGC
CTAAAGCCCGTAACACTGCAGCAGGTGCTGCCCGCGCTTGCACCGTCCGAAGAAAAGCGCGGCCT
AAAGCGCGAGTCTGGTGACTTGGCACCCACCGTGCAGCTGATGGTACCCAAGCGCCAGCGACTGG
AAGATGTCTTGGAAAAAATGACCGTGGAACCTGGGCTGGAGCCCGAGGTCCGCGTGCGGCCAATC
AAGCAGGTGGCGCCGGGACTGGGCGTGCAGACCGTGGACGTTCAGATACCCACTACCAGTAGCAC
CAGTATTGCCACCGCCACAGAGGGCATGGAGACACAAACGTCCCCGGTTGCCTCAGCGGTGGCGG
ATGCCGCGGTGCAGGCGGTCGCTGCGGCCGCGTCCAAGACCTCTACGGAGGTGCAAACGGACCCG
TGGATGTTTCGCGTTTCAGCCCCCCGGCGCCCGCGCCGTTCGAGGAAGTACGGCGCCGCCAGCGCG
CTACTGCCCGAATATGCCCTACATCCTTCCATTGCGCCTACCCCCGGCTATCGTGGCTACACCTACC
GCCCCAGAAGACGAGCAACTACCCGACGCCGAACCACCACTGGAACCCGCCGCCGCCGTCGCCGT
CGCCAGCCCGTGCTGGCCCCGATTTCCGTGCGCAGGGTGGCTCGCGAAGGAGGCAGGACCCTGGT
GCTGCCAACAGCGCGCTACCACCCCAGCATCGTTTAAAAGCCGGTCTTTGTGGTTCTTGCAGATAT
GGCCCTCACCTGCCGCCTCCGTTTCCCGGTGCCGGGATTCCGAGGAAGAATGCACCGTAGGAGGG
GCATGGCCGGCCACGGCCTGACGGGCGGCATGCGTCGTGCGCACCACCGGCGGCGGCGCGCGTCG
CACCGTCGCATGCGCGGCGGTATCCTGCCCCTCCTTATTCCACTGATCGCCGCGGCGATTGGCGCC
GTGCCCGGAATTGCATCCGTGGCCTTGCAGGCGCAGAGACACTGATTAAAAACAAGTTGCATGTG
GAAAAATCAAAATAAAAAGTCTGGACTCTCACGCTCGCTTGGTCCTGTAACTATTTTGTAGAATGG
AAGACATCAACTTTGCGTCTCTGGCCCCGCGACACGGCTCGCGCCCGTTCATGGGAAACTGGCAAG
ATATCGGCACCAGCAATATGAGCGGTGGCGCCTTCAGCTGGGGCTCGCTGTGGAGCGGCATTAAA
AATTTCGGTTCCACCGTTAAGAACTATGGCAGCAAGGCCTGGAACAGCAGCACAGGCCAGATGCT
GAGGGATAAGTTGAAAGAGCAAAATTTCCAACAAAAGGTGGTAGATGGCCTGGCCTCTGGCATTA
GCGGGGTGGTGGACCTGGCCAACCAGGCAGTGCAAAATAAGATTAACAGTAAGCTTGATCCCCGC
CCTCCCGTAGAGGAGCCTCCACCGGCCGTGGAGACAGTGTCTCCAGAGGGGCGTGGCGAAAAGCG
TCCGCGCCCCGACAGGGAAGAAACTCTGGTGACGCAAATAGACGAGCCTCCCTCGTACGAGGAGG
CACTAAAGCAAGGCCTGCCCACCACCCGTCCCATCGCGCCCATGGCTACCGGAGTGCTGGGCCAG
CACACACCCGTAACGCTGGACCTGCCTCCCCCCGCCGACACCCAGCAGAAACCTGTGCTGCCAGG
CCCGACCGCCGTTGTTGTAACCCGTCCTAGCCGCGCGTCCCTGCGCCGCGCCGCCAGCGGTCCGCG
ATCGTTGCGGCCCGTAGCCAGTGGCAACTGGCAAAGCACACTGAACAGCATCGTGGGTCTGGGGG
TGCAATCCCTGAAGCGCCGACGATGCTTCTGATAGCTAACGTGTCGTATGTGTGTCATGTATGCGT
CCATGTCGCCGCCAGAGGAGCTGCTGAGCCGCCGCGCGCCCGCTTTCCAAGATGGCTACCCCTTCG
ATGATGCCGCAGTGGTCTTACATGCACATCTCGGGCCAGGACGCCTCGGAGTACCTGAGCCCCGG
GCTGGTGCAGTTTGCCCGCGCCACCGAGACGTACTTCAGCCTGAATAACAAGTTTAGAAACCCCAC
GGTGGCGCCTACGCACGACGTGACCACAGACCGGTCCCAGCGTTTGACGCTGCGGTTCATCCCTGT
GGACCGTGAGGATACTGCGTACTCGTACAAGGCGCGGTTCACCCTAGCTGTGGGTGATAACCGTGT
GCTGGACATGGCTTCCACGTACTTTGACATCCGCGGCGTGCTGGACAGGGGCCCTACTTTTAAGCC
CTACTCTGGCACTGCCTACAACGCCCTGGCTCCCAAGGGTGCCCCAAATCCTTGCGAATGGGATGA
AGCTGCTACTGCTCTTGAAATAAACCTAGAAGAAGAGGACGATGACAACGAAGACGAAGTAGAC
GAGCAAGCTGAGCAGCAAAAAACTCACGTATTTGGGCAGGCGCCTTATTCTGGTATAAATATTAC
AAAGGAGGGTATTCAAATAGGTGTCGAAGGTCAAACACCTAAATATGCCGATAAAACATTTCAAC
CTGAACCTCAAATAGGAGAATCTCAGTGGTACGAAACAGAAATTAATCATGCAGCTGGGAGAGTC
CTAAAAAAGACTACCCCAATGAAACCATGTTACGGTTCATATGCAAAACCCACAAATGAAAATGG
AGGGCAAGGCATTCTTGTAAAGCAACAAAATGGAAAGCTAGAAAGTCAAGTGGAAATGCAATTTT
TCTCAACTACTGAGGCAGCCGCAGGCAATGGTGATAACTTGACTCCTAAAGTGGTATTGTACAGTG
AAGATGTAGATATAGAAACCCCAGACACTCATATTTCTTACATGCCCACTATTAAGGAAGGTAACT
CACGAGAACTAATGGGCCAACAATCTATGCCCAACAGGCCTAATTACATTGCTTTTAGGGACAATT
TTATTGGTCTAATGTATTACAACAGCACGGGTAATATGGGTGTTCTGGCGGGCCAAGCATCGCAGT
TGAATGCTGTTGTAGATTTGCAAGACAGAAACACAGAGCTTTCATACCAGCTTTTGCTTGATTCCA
TTGGTGATAGAACCAGGTACTTTTCTATGTGGAATCAGGCTGTTGACAGCTATGATCCAGATGTTA
GAATTATTGAAAATCATGGAACTGAAGATGAACTTCCAAATTACTGCTTTCCACTGGGAGGTGTGA
TTAATACAGAGACTCTTACCAAGGTAAAACCTAAAACAGGTCAGGAAAATGGATGGGAAAAAGAT
GCTACAGAATTTTCAGATAAAAATGAAATAAGAGTTGGAAATAATTTTGCCATGGAAATCAATCT
AAATGCCAACCTGTGGAGAAATTTCCTGTACTCCAACATAGCGCTGTATTTGCCCGACAAGCTAAA
GTACAGTCCTTCCAACGTAAAAATTTCTGATAACCCAAACACCTACGACTACATGAACAAGCGAGT
GGTGGCTCCCGGGCTAGTGGACTGCTACATTAACCTTGGAGCACGCTGGTCCCTTGACTATATGGA
CAACGTCAACCCATTTAACCACCACCGCAATGCTGGCCTGCGCTACCGCTCAATGTTGCTGGGCAA
TGGTCGCTATGTGCCCTTCCACATCCAGGTGCCTCAGAAGTTCTTTGCCATTAAAAACCTCCTTCTC
CTGCCGGGCTCATACACCTACGAGTGGAACTTCAGGAAGGATGTTAACATGGTTCTGCAGAGCTCC
CTAGGAAATGACCTAAGGGTTGACGGAGCCAGCATTAAGTTTGATAGCATTTGCCTTTACGCCACC
TTCTTCCCCATGGCCCACAACACCGCCTCCACGCTTGAGGCCATGCTTAGAAACGACACCAACGAC
CAGTCCTTTAACGACTATCTCTCCGCCGCCAACATGCTCTACCCTATACCCGCCAACGCTACCAAC
GTGCCCATATCCATCCCCTCCCGCAACTGGGCGGCTTTCCGCGGCTGGGCCTTCACGCGCCTTAAG
ACTAAGGAAACCCCATCACTGGGCTCGGGCTACGACCCTTATTACACCTACTCTGGCTCTATACCC
TACCTAGATGGAACCTTTTACCTCAACCACACCTTTAAGAAGGTGGCCATTACCTTTGACTCTTCTG
TCAGCTGGCCTGGCAATGACCGCCTGCTTACCCCCAACGAGTTTGAAATTAAGCGCTCAGTTGACG
GGGAGGGTTACAACGTTGCCCAGTGTAACATGACCAAAGACTGGTTCCTGGTACAAATGCTAGCT
AACTATAACATTGGCTACCAGGGCTTCTATATCCCAGAGAGCTACAAGGACCGCATGTACTCCTTC
TTTAGAAACTTCCAGCCCATGAGCCGTCAGGTGGTGGATGATACTAAATACAAGGACTACCAACA
GGTGGGCATCCTACACCAACACAACAACTCTGGATTTGTTGGCTACCTTGCCCCCACCATGCGCGA
AGGACAGGCCTACCCTGCTAACTTCCCCTATCCGCTTATAGGCAAGACCGCAGTTGACAGCATTAC
CCAGAAAAAGTTTCTTTGCGATCGCACCCTTTGGCGCATCCCATTCTCCAGTAACTTTATGTCCATG
GGCGCACTCACAGACCTGGGCCAAAACCTTCTCTACGCCAACTCCGCCCACGCGCTAGACATGACT
TTTGAGGTGGATCCCATGGACGAGCCCACCCTTCTTTATGTTTTGTTTGAAGTCTTTGACGTGGTCC
GTGTGCACCAGCCGCACCGCGGCGTCATCGAAACCGTGTACCTGCGCACGCCCTTCTCGGCCGGCA
ACGCCACAACATAAAGAAGCAAGCAACATCAACAACAGCTGCCGCCATGGGCTCCAGTGAGCAG
GAACTGAAAGCCATTGTCAAAGATCTTGGTTGTGGGCCATATTTTTTGGGCACCTATGACAAGCGC
TTTCCAGGCTTTGTTTCTCCACACAAGCTCGCCTGCGCCATAGTCAATACGGCCGGTCGCGAGACT
GGGGGCGTACACTGGATGGCCTTTGCCTGGAACCCGCACTCAAAAACATGCTACCTCTTTGAGCCC
TTTGGCTTTTCTGACCAGCGACTCAAGCAGGTTTACCAGTTTGAGTACGAGTCACTCCTGCGCCGT
AGCGCCATTGCTTCTTCCCCCGACCGCTGTATAACGCTGGAAAAGTCCACCCAAAGCGTACAGGGG
CCCAACTCGGCCGCCTGTGGACTATTCTGCTGCATGTTTCTCCACGCCTTTGCCAACTGGCCCCAAA
CTCCCATGGATCACAACCCCACCATGAACCTTATTACCGGGGTACCCAACTCCATGCTCAACAGTC
CCCAGGTACAGCCCACCCTGCGTCGCAACCAGGAACAGCTCTACAGCTTCCTGGAGCGCCACTCGC
CCTACTTCCGCAGCCACAGTGCGCAGATTAGGAGCGCCACTTCTTTTTGTCACTTGAAAAACATGT
AAAAATAATGTACTAGAGACACTTTCAATAAAGGCAAATGCTTTTATTTGTACACTCTCGGGTGAT
TATTTACCCCCACCCTTGCCGTCTGCGCCGTTTAAAAATCAAAGGGGTTCTGCCGCGCATCGCTAT
GCGCCACTGGCAGGGACACGTTGCGATACTGGTGTTTAGTGCTCCACTTAAACTCAGGCACAACCA
TCCGCGGCAGCTCGGTGAAGTTTTCACTCCACAGGCTGCGCACCATCACCAACGCGTTTAGCAGGT
CGGGCGCCGATATCTTGAAGTCGCAGTTGGGGCCTCCGCCCTGCGCGCGCGAGTTGCGATACACA
GGGTTGCAGCACTGGAACACTATCAGCGCCGGGTGGTGCACGCTGGCCAGCACGCTCTTGTCGGA
GATCAGATCCGCGTCCAGGTCCTCCGCGTTGCTCAGGGCGAACGGAGTCAACTTTGGTAGCTGCCT
TCCCAAAAAGGGCGCGTGCCCAGGCTTTGAGTTGCACTCGCACCGTAGTGGCATCAAAAGGTGAC
CGTGCCCGGTCTGGGCGTTAGGATACAGCGCCTGCATAAAAGCCTTGATCTGCTTAAAAGCCACCT
GAGCCTTTGCGCCTTCAGAGAAGAACATGCCGCAAGACTTGCCGGAAAACTGATTGGCCGGACAG
GCCGCGTCGTGCACGCAGCACCTTGCGTCGGTGTTGGAGATCTGCACCACATTTCGGCCCCACCGG
TTCTTCACGATCTTGGCCTTGCTAGACTGCTCCTTCAGCGCGCGCTGCCCGTTTTCGCTCGTCACAT
CCATTTCAATCACGTGCTCCTTATTTATCATAATGCTTCCGTGTAGACACTTAAGCTCGCCTTCGAT
CTCAGCGCAGCGGTGCAGCCACAACGCGCAGCCCGTGGGCTCGTGATGCTTGTAGGTCACCTCTGC
AAACGACTGCAGGTACGCCTGCAGGAATCGCCCCATCATCGTCACAAAGGTCTTGTTGCTGGTGAA
GGTCAGCTGCAACCCGCGGTGCTCCTCGTTCAGCCAGGTCTTGCATACGGCCGCCAGAGCTTCCAC
TTGGTCAGGCAGTAGTTTGAAGTTCGCCTTTAGATCGTTATCCACGTGGTACTTGTCCATCAGCGCG
CGCGCAGCCTCCATGCCCTTCTCCCACGCAGACACGATCGGCACACTCAGCGGGTTCATCACCGTA
ATTTCACTTTCCGCTTCGCTGGGCTCTTCCTCTTCCTCTTGCGTCCGCATACCACGCGCCACTGGGT
CGTCTTCATTCAGCCGCCGCACTGTGCGCTTACCTCCTTTGCCATGCTTGATTAGCACCGGTGGGTT
GCTGAAACCCACCATTTGTAGCGCCACATCTTCTCTTTCTTCCTCGCTGTCCACGATTACTAATACG
ACTCACTATAGGTGTGGAATTTCACAGGAGGTACAGCTATGACCATGATTACGGATTCACTGGCCG
TCGTTTTACAACGTCGTGACTGGGAAAACCCTGGCGTTACCCAACTTAATCGCCTTGCAGCACATC
CCCCTTTCGCCAGCTGGCGTAATAGCGAAGAGGCCCGCACCGATCGCCCTTCCCAACAGTTGCGCA
GCCTGAATGGCGAATAGGTCGCGCCGCACCGCGTCCGCGCTCGGGGGTGGTTTCGCGCTGCTCCTC
TTCCCGACTGGCCATTTCCTTCTCCTATAGGCAGAAAAAGATCATGGAGTCAGTCGAGAAGAAGG
ACAGCCTAACCGCCCCCTCTGAGTTCGCCACCACCGCCTCCACCGATGCCGCCAACGCGCCTACCA
CCTTCCCCGTCGAGGCACCCCCGCTTGAGGAGGAGGAAGTGATTATCGAGCAGGACCCAGGTTTT
GTAAGCGAAGACGACGAGGACCGCTCAGTACCAACAGAGGATAAAAAGCAAGACCAGGACAACG
CAGAGGCAAACGAGGAACAAGTCGGGCGGGGGGACGAAAGGCATGGCGACTACCTAGATGTGGG
AGACGACGTGCTGTTGAAGCATCTGCAGCGCCAGTGCGCCATTATCTGCGACGCGTTGCAAGAGC
GCAGCGATGTGCCCCTCGCCATAGCGGATGTCAGCCTTGCCTACGAACGCCACCTATTCTCACCGC
GCGTACCCCCCAAACGCCAAGAAAACGGCACATGCGAGCCCAACCCGCGCCTCAACTTCTACCCC
GTATTTGCCGTGCCAGAGGTGCTTGCCACCTATCACATCTTTTTCCAAAACTGCAAGATACCCCTAT
CCTGCCGTGCCAACCGCAGCCGAGCGGACAAGCAGCTGGCCTTGCGGCAGGGCGCTGTCATACCT
GATATCGCCTCGCTCAACGAAGTGCCAAAAATCTTTGAGGGTCTTGGACGCGACGAGAAGCGCGC
GGCAAACGCTCTGCAACAGGAAAACAGCGAAAATGAAAGTCACTCTGGAGTGTTGGTGGAACTCG
AGGGTGACAACGCGCGCCTAGCCGTACTAAAACGCAGCATCGAGGTCACCCACTTTGCCTACCCG
GCACTTAACCTACCCCCCAAGGTCATGAGCACAGTCATGAGTGAGCTGATCGTGCGCCGTGCGCA
GCCCCTGGAGAGGGATGCAAATTTGCAAGAACAAACAGAGGAGGGCCTACCCGCAGTTGGCGAC
GAGCAGCTAGCGCGCTGGCTTCAAACGCGCGAGCCTGCCGACTTGGAGGAGCGACGCAAACTAAT
GATGGCCGCAGTGCTCGTTACCGTGGAGCTTGAGTGCATGCAGCGGTTCTTTGCTGACCCGGAGAT
GCAGCGCAAGCTAGAGGAAACATTGCACTACACCTTTCGACAGGGCTACGTACGCCAGGCCTGCA
AGATCTCCAACGTGGAGCTCTGCAACCTGGTCTCCTACCTTGGAATTTTGCACGAAAACCGCCTTG
GGCAAAACGTGCTTCATTCCACGCTCAAGGGCGAGGCGCGCCGCGACTACGTCCGCGACTGCGTTT
ACTTATTTCTATGCTACACCTGGCAGACGGCCATGGGCGTTTGGCAGCAGTGCTTGGAGGAGTGCA
ACCTCAAGGAGCTGCAGAAACTGCTAAAGCAAAACTTGAAGGACCTATGGACGGCCTTCAACGAG
CGCTCCGTGGCCGCGCACCTGGCGGACATCATTTTCCCCGAACGCCTGCTTAAAACCCTGCAACAG
GGTCTGCCAGACTTCACCAGTCAAAGCATGTTGCAGAACTTTAGGAACTTTATCCTAGAGCGCTCA
GGAATCTTGCCCGCCACCTGCTGTGCACTTCCTAGCGACTTTGTGCCCATTAAGTACCGCGAATGC
CCTCCGCCGCTTTGGGGCCACTGCTACCTTCTGCAGCTAGCCAACTACCTTGCCTACCACTCTGACA
TAATGGAAGACGTGAGCGGTGACGGTCTACTGGAGTGTCACTGTCGCTGCAACCTATGCACCCCGC
ACCGCTCCCTGGTTTGCAATTCGCAGCTGCTTAACGAAAGTCAAATTATCGGTACCTTTGAGCTGC
AGGGTCCCTCGCCTGACGAAAAGTCCGCGGCTCCGGGGTTGAAACTCACTCCGGGGCTGTGGACG
TCGGCTTACCTTCGCAAATTTGTACCTGAGGACTACCACGCCCACGAGATTAGGTTCTACGAAGAC
CAATCCCGCCCGCCTAATGCGGAGCTTACCGCCTGCGTCATTACCCAGGGCCACATTCTTGGCCAA
TTGCAAGCCATCAACAAAGCCCGCCAAGAGTTTCTGCTACGAAAGGGACGGGGGGTTTACTTGGA
CCCCCAGTCCGGCGAGGAGCTCAACCCAATCCCCCCGCCGCCGCAGCCCTATCAGCAGCAGCCGC
GGGCCCTTGCTTCCCAGGATGGCACCCAAAAAGAAGCTGCAGCTGCCGCCGCCACCCACGGACGA
GGAGGAATACTGGGACAGTCAGGCAGAGGAGGTTTTGGACGAGGAGGAGGAGGACATGATGGAA
GACTGGGAGAGCCTAGACGAGGAAGCTTCCGAGGTCGAAGAGGTGTCAGACGAAACACCGTCAC
CCTCGGTCGCATTCCCCTCGCCGGCGCCCCAGAAATCGGCAACCGGTTCCAGCATGGCTACAACCT
CCGCTCCTCAGGCGCCGCCGGCACTGCCCGTTCGCCGACCCAACCGTAGATGGGACACCACTGGA
ACCAGGGCCGGTAAGTCCAAGCAGCCGCCGCCGTTAGCCCAAGAGCAACAACAGCGCCAAGGCTA
CCGCTCATGGCGCGGGCACAAGAACGCCATAGTTGCTTGCTTGCAAGACTGTGGGGGCAACATCT
CCTTCGCCCGCCGCTTTCTTCTCTACCATCACGGCGTGGCCTTCCCCCGTAACATCCTGCATTACTA
CCGTCATCTCTACAGCCCATACTGCACCGGCGGCAGCGGCAGCAACAGCAGCGGCCACACAGAAG
CAAAGGCGACCGGATAGCAAGACTCTGACAAAGCCCAAGAAATCCACAGCGGCGGCAGCAGCAG
GAGGAGGAGCGCTGCGTCTGGCGCCCAACGAACCCGTATCGACCCGCGAGCTTAGAAACAGGATT
TTTCCCACTCTGTATGCTATATTTCAACAGAGCAGGGGCCAAGAACAAGAGCTGAAAATAAAAAA
CAGGTCTCTGCGATCCCTCACCCGCAGCTGCCTGTATCACAAAAGCGAAGATCAGCTTCGGCGCAC
GCTGGAAGACGCGGAGGCTCTCTTCAGTAAATACTGCGCGCTGACTCTTAAGGACTAGTTTCGCGC
CCTTTCTCAAATTTAAGCGCGAAAACTACGTCATCTCCAGCGGCCACACCCGGCGCCAGCACCTGT
TGTCAGCGCCATTATGAGCAAGGAAATTCCCACGCCCTACATGTGGAGTTACCAGCCACAAATGG
GACTTGCGGCTGGAGCTGCCCAAGACTACTCAACCCGAATAAACTACATGAGCGCGGGACCCCAC
ATGATATCCCGGGTCAACGGAATACGCGCCCACCGAAACCGAATTCTCCTGGAACAGGCGGCTAT
TACCACCACACCTCGTAATAACCTTAATCCCCGTAGTTGGCCCGCTGCCCTGGTGTACCAGGAAAG
TCCCGCTCCCACCACTGTGGTACTTCCCAGAGACGCCCAGGCCGAAGTTCAGATGACTAACTCAGG
GGCGCAGCTTGCGGGCGGCTTTCGTCACAGGGTGCGGTCGCCCGGGCAGGGTATAACTCACCTGA
CAATCAGAGGGCGAGGTATTCAGCTCAACGACGAGTCGGTGAGCTCCTCGCTTGGTCTCCGTCCGG
ACGGGACATTTCAGATCGGCGGCGCCGGCCGCTCTTCATTCACGCCTCGTCAGGCAATCCTAACTC
TGCAGACCTCGTCCTCTGAGCCGCGCTCTGGAGGCATTGGAACTCTGCAATTTATTGAGGAGTTTG
TGCCATCGGTCTACTTTAACCCCTTCTCGGGACCTCCCGGCCACTATCCGGATCAATTTATTCCTAA
CTTTGACGCGGTAAAGGACTCGGCGGACGGCTACGACTGAATGTTAAGTGGAGAGGCAGAGCAAC
TGCGCCTGAAACACCTGGTCCACTGTCGCCGCCACAAGTGCTTTGCCCGCGACTCCGGTGAGTTTT
GCTACTTTGAATTGCCCGAGGATCATATCGAGGGCCCGGCGCACGGCGTCCGGCTTACCGCCCAGG
GAGAGCTTGCCCGTAGCCTGATTCGGGAGTTTACCCAGCGCCCCCTGCTAGTTGAGCGGGACAGG
GGACCCTGTGTTCTCACTGTGATTTGCAACTGTCCTAACCCTGGATTACATCAAGATCTTTGTTGCC
ATCTCTGTGCTGAGTATAATAAATACAGAAATTAAAATATACTGGGGCTCCTATCGCCATCCTGTA
AACGCCACCGTCTTCACCCGCCCAAGCAAACCAAGGCGAACCTTACCTGGTACTTTTAACATCTCT
CCCTCTGTGATTTACAACAGTTTCAACCCAGACGGAGTGAGTCTACGAGAGAACCTCTCCGAGCTC
AGCTACTCCATCAGAAAAAACACCACCCTCCTTACCTGCCGGGAACGTACGAGTGCGTCACCGGC
CGCTGCACCACACCTACCGCCTGACCGTAAACCAGACTTTTTCCGGACAGACCTCAATAACTCTGT
TTACCAGAACAGGAGGTGAGCTTAGAAAACCCTTAGGGTATTAGGCCAAAGGCGCAGCTACTGTG
GGGTTTATGAACAATTCAAGCAACTCTACGGGCTATTCTAATTCAGGTTTCTCTAGAAATGGACGG
AATTATTACAGAGCAGCGCCTGCTAGAAAGACGCAGGGCAGCGGCCGAGCAACAGCGCATGAATC
AAGAGCTCCAAGACATGGTTAACTTGCACCAGTGCAAAAGGGGTATCTTTTGTCTGGTAAAGCAG
GCCAAAGTCACCTACGACAGTAATACCACCGGACACCGCCTTAGCTACAAGTTGCCAACCAAGCG
TCAGAAATTGGTGGTCATGGTGGGAGAAAAGCCCATTACCATAACTCAGCACTCGGTAGAAACCG
AAGGCTGCATTCACTCACCTTGTCAAGGACCTGAGGATCTCTGCACCCTTATTAAGACCCTGTGCG
GTCTCAAAGATCTTATTCCCTTTAACTAATAAAAAAAAATAATAAAGCATCACTTACTTAAAATCA
GTTAGCAAATTTCTGTCCAGTTTATTCAGCAGCACCTCCTTGCCCTCCTCCCAGCTCTGGTATTGCA
GCTTCCTCCTGGCTGCAAACTTTCTCCACAATCTAAATGGAATGTCAGTTTCCTCCTGTTCCTGTCC
ATCCGCACCCACTATCTTCATGTTGTTGCAGATGAAGCGCGCAAGACCGTCTGAAGATACCTTCAA
CCCCGTGTATCCATATGACACGGAAACCGGTCCTCCAACTGTGCCTTTTCTTACTCCTCCCTTTGTA
TCCCCCAATGGGTTTCAAGAGAGTCCCCCTGGGGTACTCTCTTTGCGCCTATCCGAACCTCTAGTTA
CCTCCAATGGCATGCTTGCGCTCAAAATGGGCAACGGCCTCTCTCTGGACGAGGCCGGCAACCTTA
CCTCCCAAAATGTAACCACTGTGAGCCCACCTCTCAAAAAAACCAAGTCAAACATAAACCTGGAA
ATATCTGCACCCCTCACAGTTACCTCAGAAGCCCTAACTGTGGCTGCCGCCGCACCTCTAATGGTC
GCGGGCAACACACTCACCATGCAATCACAGGCCCCGCTAACCGTGCACGACTCCAAACTTAGCAT
TGCCACCCAAGGACCCCTCACAGTGTCAGAAGGAAAGCTAGCCCTGCAAACATCAGGCCCCCTCA
CCACCACCGATAGCAGTACCCTTACTATCACTGCCTCACCCCCTCTAACTACTGCCACTGGTAGCTT
GGGCATTGACTTGAAAGAGCCCATTTATACACAAAATGGAAAACTAGGACTAAAGTACGGGGCTC
CTTTGCATGTAACAGACGACCTAAACACTTTGACCGTAGCAACTGGTCCAGGTGTGACTATTAATA
ATACTTCCTTGCAAACTAAAGTTACTGGAGCCTTGGGTTTTGATTCACAAGGCAATATGCAACTTA
ATGTAGCAGGAGGACTAAGGATTGATTCTCAAAACAGACGCCTTATACTTGATGTTAGTTATCCGT
TTGATGCTCAAAACCAACTAAATCTAAGACTAGGACAGGGCCCTCTTTTTATAAACTCAGCCCACA
ACTTGGATATTAACTACAACAAAGGCCTTTACTTGTTTACAGCTTCAAACAATTCCAAAAAGCTTG
AGGTTAACCTAAGCACTGCCAAGGGGTTGATGTTTGACGCTACAGCCATAGCCATTAATGCAGGA
GATGGGCTTGAATTTGGTTCACCTAATGCACCAAACACAAATCCCCTCAAAACAAAAATTGGCCAT
GGCCTAGAATTTGATTCAAACAAGGCTATGGTTCCTAAACTAGGAACTGGCCTTAGTTTTGACAGC
ACAGGTGCCATTACAGTAGGAAACAAAAATAATGATAAGCTAACTTTGTGGACCACACCAGCTCC
ATCTCCTAACTGTAGACTAAATGCAGAGAAAGATGCTAAACTCACTTTGGTCTTAACAAAATGTGG
CAGTCAAATACTTGCTACAGTTTCAGTTTTGGCTGTTAAAGGCAGTTTGGCTCCAATATCTGGAAC
AGTTCAAAGTGCTCATCTTATTATAAGATTTGACGAAAATGGAGTGCTACTAAACAATTCCTTCCT
GGACCCAGAATATTGGAACTTTAGAAATGGAGATCTTACTGAAGGCACAGCCTATACAAACGCTG
TTGGATTTATGCCTAACCTATCAGCTTATCCAAAATCTCACGGTAAAACTGCCAAAAGTAACATTG
TCAGTCAAGTTTACTTAAACGGAGACAAAACTAAACCTGTAACACTAACCATTACACTAAACGGT
ACACAGGAAACAGGAGACACAACTCCAAGTGCATACTCTATGTCATTTTCATGGGACTGGTCTGGC
CACAACTACATTAATGAAATATTTGCCACATCCTCTTACACTTTTTCATACATTGCCCAAGAATAAA
GAATCGTTTGTGTTATGTTTCAACGTGTTTATTTTTCAATTGCAGAAAATTTCGAATCATTTTTCATT
CAGTAGTATAGCCCCACCACCACATAGCTTATACAGATCACCGTACCTTAATCAAACTCACAGAAC
CCTAGTATTCAACCTGCCACCTCCCTCCCAACACACAGAGTACACAGTCCTTTCTCCCCGGCTGGC
CTTAAAAAGCATCATATCATGGGTAACAGACATATTCTTAGGTGTTATATTCCACACGGTTTCCTGT
CGAGCCAAACGCTCATCAGTGATATTAATAAACTCCCCGGGCAGCTCACTTAAGTTCATGTCGCTG
TCCAGCTGCTGAGCCACAGGCTGCTGTCCAACTTGCGGTTGCTTAACGGGCGGCGAAGGAGAAGT
CCACGCCTACATGGGGGTAGAGTCATAATCGTGCATCAGGATAGGGCGGTGGTGCTGCAGCAGCG
CGCGAATAAACTGCTGCCGCCGCCGCTCCGTCCTGCAGGAATACAACATGGCAGTGGTCTCCTCAG
CGATGATTCGCACCGCCCGCAGCATAAGGCGCCTTGTCCTCCGGGCACAGCAGCGCACCCTGATCT
CACTTAAATCAGCACAGTAACTGCAGCACAGCACCACAATATTGTTCAAAATCCCACAGTGCAAG
GCGCTGTATCCAAAGCTCATGGCGGGGACCACAGAACCCACGTGGCCATCATACCACAAGCGCAG
GTAGATTAAGTGGCGACCCCTCATAAACACGCTGGACATAAACATTACCTCTTTTGGCATGTTGTA
ATTCACCACCTCCCGGTACCATATAAACCTCTGATTAAACATGGCGCCATCCACCACCATCCTAAA
CCAGCTGGCCAAAACCTGCCCGCCGGCTATACACTGCAGGGAACCGGGACTGGAACAATGACAGT
GGAGAGCCCAGGACTCGTAACCATGGATCATCATGCTCGTCATGATATCAATGTTGGCACAACAC
AGGCACACGTGCATACACTTCCTCAGGATTACAAGCTCCTCCCGCGTTAGAACCATATCCCAGGGA
ACAACCCATTCCTGAATCAGCGTAAATCCCACACTGCAGGGAAGACCTCGCACGTAACTCACGTTG
TGCATTGTCAAAGTGTTACATTCGGGCAGCAGCGGATGATCCTCCAGTATGGTAGCGCGGGTTTCT
GTCTCAAAAGGAGGTAGACGATCCCTACTGTACGGAGTGCGCCGAGACAACCGAGATCGTGTTGG
TCGTAGTGTCATGCCAAATGGAACGCCGGACGTAGTCATATTTCCTGAAGCAAAACCAGGTGCGG
GCGTGACAAACAGATCTGCGTCTCCGGTCTCGCCGCTTAGATCGCTCTGTGTAGTAGTTGTAGTAT
ATCCACTCTCTCAAAGCATCCAGGCGCCCCCTGGCTTCGGGTTCTATGTAAACTCCTTCATGCGCCG
CTGCCCTGATAACATCCACCACCGCAGAATAAGCCACACCCAGCCAACCTACACATTCGTTCTGCG
AGTCACACACGGGAGGAGCGGGAAGAGCTGGAAGAACCATGTTTTTTTTTTTATTCCAAAAGATTA
TCCAAAACCTCAAAATGAAGATCTATTAAGTGAACGCGCTCCCCTCCGGTGGCGTGGTCAAACTCT
ACAGCCAAAGAACAGATAATGGCATTTGTAAGATGTTGCACAATGGCTTCCAAAAGGCAAACGGC
CCTCACGTCCAAGTGGACGTAAAGGCTAAACCCTTCAGGGTGAATCTCCTCTATAAACATTCCAGC
ACCTTCAACCATGCCCAAATAATTCTCATCTCGCCACCTTCTCAATATATCTCTAAGCAAATCCCGA
ATATTAAGTCCGGCCATTGTAAAAATCTGCTCCAGAGCGCCCTCCACCTTCAGCCTCAAGCAGCGA
ATCATGATTGCAAAAATTCAGGTTCCTCACAGACCTGTATAAGATTCAAAAGCGGAACATTAACA
AAAATACCGCGATCCCGTAGGTCCCTTCGCAGGGCCAGCTGAACATAATCGTGCAGGTCTGCACG
GACCAGCGCGGCCACTTCCCCGCCAGGAACCATGACAAAAGAACCCACACTGATTATGACACGCA
TACTCGGAGCTATGCTAACCAGCGTAGCCCCGATGTAAGCTTGTTGCATGGGCGGCGATATAAAAT
GCAAGGTGCTGCTCAAAAAATCAGGCAAAGCCTCGCGCAAAAAAGAAAGCACATCGTAGTCATGC
TCATGCAGATAAAGGCAGGTAAGCTCCGGAACCACCACAGAAAAAGACACCATTTTTCTCTCAAA
CATGTCTGCGGGTTTCTGCATAAACACAAAATAAAATAACAAAAAAACATTTAAACATTAGAAGC
CTGTCTTACAACAGGAAAAACAACCCTTATAAGCATAAGACGGACTACGGCCATGCCGGCGTGAC
CGTAAAAAAACTGGTCACCGTGATTAAAAAGCACCACCGACAGCTCCTCGGTCATGTCCGGAGTC
ATAATGTAAGACTCGGTAAACACATCAGGTTGATTCACATCGGTCAGTGCTAAAAAGCGACCGAA
ATAGCCCGGGGGAATACATACCCGCAGGCGTAGAGACAACATTACAGCCCCCATAGGAGGTATAA
CAAAATTAATAGGAGAGAAAAACACATAAACACCTGAAAAACCCTCCTGCCTAGGCAAAATAGCA
CCCTCCCGCTCCAGAACAACATACAGCGCTTCCACAGCGGCAGCCATAACAGTCAGCCTTACCAGT
AAAAAAGAAAACCTATTAAAAAAACACCACTCGACACGGCACCAGCTCAATCAGTCACAGTGTAA
AAAAGGGCCAAGTGCAGAGCGAGTATATATAGGACTAAAAAATGACGTAACGGTTAAAGTCCAC
AAAAAACACCCAGAAAACCGCACGCGAACCTACGCCCAGAAACGAAAGCCAAAAAACCCACAAC
TTCCTCAAATCGTCACTTCCGTTTTCCCACGTTACGTCACTTCCCATTTTAAGAAAACTACAATTCC
CAACACATACAAGTTACTCCGCCCTAAAACCTACGTCACCCGCCCCGTTCCCACGCCCCGCGCCAC
GTCACAAACTCCACCCCCTCATTATCATATTGGCTTCAATCCAAAATAAGGTATATTATTGATGAT
GTTAATTAATTTAAATCCGCATGCGATATCGAGCTCTCCCGGGAATTCGGATCTGCGACGCGAGGC
TGGATGGCCTTCCCCATTATGATTCTTCTCGCGTTTAAGGGCACCAATAACTGCCTTAAAAAAATT
ACGCCCCGCCCTGCCACTCATCGCAGTACTGTTGTAATTCATTAAGCATTCTGCCGACATGGAAGC
CATCACAAACGGCATGATGAACCTGAATCGCCAGCGGCATCAGCACCTTGTCGCCTTGCGTATAAT
ATTTGCCCATGGTGAAAACGGGGGCGAAGAAGTTGTCCATATTGGCCACGTTTAAATCAAAACTG
GTGAAACTCACCCAGGGATTGGCTGAGACGAAAAACATATTCTCAATAAACCCTTTAGGGAAATA
GGCCAGGTTTTCACCGTAACACGCCACATCTTGCGAATATATGTGTAGAAACTGCCGGAAATCGTC
GTGGTATTCACTCCAGAGCGATGAAAACGTTTCAGTTTGCTCATGGAAAACGGTGTAACAAGGGT
GAACACTATCCCATATCACCAGCTCACCGTCTTTCATTGCCATACGGAATTCCGGATGAGCATTCA
TCAGGCGGGCAAGAATGTGAATAAAGGCCGGATAAAACTTGTGCTTATTTTTCTTTACGGTCTTTA
AAAAGGCCGTAATATCCAGCTGAACGGTCTGGTTATAGGTACATTGAGCAACTGACTGAAATGCC
TCAAAATGTTCTTTACGATGCCATTGGGATATATCAACGGTGGTATATCCAGTGATTTTTTTCTCCA
TTTTAGCTTCCTTAGCTCCTGAAAATCTCGATAACTCAAAAAATACGCCCGGTAGTGATCTTATTTC
ATTATGGTGAAAGTTGGAACCTCTTACGTGCCGATCAACGTCTCATTTTCGCCAAAAGTTGGCCCA
GGGCTTCCCGGTATCAACAGGGACACCAGGATTTATTTATTCTGCGAAGTGATCTTCCGTCACAGG
TATTTATTCGCGATAAGCTCATGGAGCGGCGTAACCGTCGCACAGGAAGGACAGAGAAAGCGCGG
ATCTGGGAAGTGACGGACAGAACGGTCAGGACCTGGATTGGGGAGGCGGTTGCCGCCGCTGCTGC
TGACGGTGTGACGTTCTCTGTTCCGGTCACACCACATACGTTCCGCCATTCCTATGCGATGCACATG
CTGTATGCCGGTATACCGCTGAAAGTTCTGCAAAGCCTGATGGGACATAAGTCCATCAGTTCAACG
GAAGTCTACACGAAGGTTTTTGCGCTGGATGTGGCTGCCCGGCACCGGGTGCAGTTTGCGATGCCG
GAGTCTGATGCGGTTGCGATGCTGAAACAATTATCCTGAGAATAAATGCCTTGGCCTTTATATGGA
AATGTGGAACTGAGTGGATATGCTGTTTTTGTCTGTTAAACAGAGAAGCTGGCTGTTATCCACTGA
GAAGCGAACGAAACAGTCGGGAAAATCTCCCATTATCGTAGAGATCCGCATTATTAATCTCAGGA
GCCTGTGTAGCGTTTATAGGAAGTAGTGTTCTGTCATGATGCCTGCAAGCGGTAACGAAAACGATT
TGAATATGCCTTCAGGAACAATAGAAATCTTCGTGCGGTGTTACGTTGAAGTGGAGCGGATTATGT
CAGCAATGGACAGAACAACCTAATGAACACAGAACCATGATGTGGTCTGTCCTTTTACAGCCAGT
AGTGCTCGCCGCAGTCGAGCGACAGGGCGAAGCCCTCGAGTGAGCGAGGAAGCACCAGGGAACA
GCACTTATATATTCTGCTTACACACGATGCCTGAAAAAACTTCCCTTGGGGTTATCCACTTATCCAC
GGGGATATTTTTATAATTATTTTTTTTATAGTTTTTAGATCTTCTTTTTTAGAGCGCCTTGTAGGCCT
TTATCCATGCTGGTTCTAGAGAAGGTGTTGTGACAAATTGCCCTTTCAGTGTGACAAATCACCCTC
AAATGACAGTCCTGTCTGTGACAAATTGCCCTTAACCCTGTGACAAATTGCCCTCAGAAGAAGCTG
TTTTTTCACAAAGTTATCCCTGCTTATTGACTCTTTTTTATTTAGTGTGACAATCTAAAAACTTGTCA
CACTTCACATGGATCTGTCATGGCGGAAACAGCGGTTATCAATCACAAGAAACGTAAAAATAGCC
CGCGAATCGTCCAGTCAAACGACCTCACTGAGGCGGCATATAGTCTCTCCCGGGATCAAAAACGT
ATGCTGTATCTGTTCGTTGACCAGATCAGAAAATCTGATGGCACCCTACAGGAACATGACGGTATC
TGCGAGATCCATGTTGCTAAATATGCTGAAATATTCGGATTGACCTCTGCGGAAGCCAGTAAGGAT
ATACGGCAGGCATTGAAGAGTTTCGCGGGGAAGGAAGTGGTTTTTTATCGCCCTGAAGAGGATGC
CGGCGATGAAAAAGGCTATGAATCTTTTCCTTGGTTTATCAAACGTGCGCACAGTCCATCCAGAGG
GCTTTACAGTGTACATATCAACCCATATCTCATTCCCTTCTTTATCGGGTTACAGAACCGGTTTACG
CAGTTTCGGCTTAGTGAAACAAAAGAAATCACCAATCCGTATGCCATGCGTTTATACGAATCCCTG
TGTCAGTATCGTAAGCCGGATGGCTCAGGCATCGTCTCTCTGAAAATCGACTGGATCATAGAGCGT
TACCAGCTGCCTCAAAGTTACCAGCGTATGCCTGACTTCCGCCGCCGCTTCCTGCAGGTCTGTGTTA
ATGAGATCAACAGCAGAACTCCAATGCGCCTCTCATACATTGAGAAAAAGAAAGGCCGCCAGACG
ACTCATATCGTATTTTCCTTCCGCGATATCACTTCCATGACGACAGGATAGTCTGAGGGTTATCTGT
CACAGATTTGAGGGTGGTTCGTCACATTTGTTCTGACCTACTGAGGGTAATTTGTCACAGTTTTGCT
GTTTCCTTCAGCCTGCATGGATTTTCTCATACTTTTTGAACTGTAATTTTTAAGGAAGCCAAATTTG
AGGGCAGTTTGTCACAGTTGATTTCCTTCTCTTTCCCTTCGTCATGTGACCTGATATCGGGGGTTAG
TTCGTCATCATTGATGAGGGTTGATTATCACAGTTTATTACTCTGAATTGGCTATCCGCGTGTGTAC
CTCTACCTGGAGTTTTTCCCACGGTGGATATTTCTTCTTGCGCTGAGCGTAAGAGCTATCTGACAGA
ACAGTTCTTCTTTGCTTCCTCGCCAGTTCGCTCGCTATGCTCGGTTACACGGCTGCGGCGAGCGCTA
GTGATAATAAGTGACTGAGGTATGTGCTCTTCTTATCTCCTTTTGTAGTGTTGCTCTTATTTTAAAC
AACTTTGCGGTTTTTTGATGACTTTGCGATTTTGTTGTTGCTTTGCAGTAAATTGCAAGATTTAATA
AAAAAACGCAAAGCAATGATTAAAGGATGTTCAGAATGAAACTCATGGAAACACTTAACCAGTGC
ATAAACGCTGGTCATGAAATGACGAAGGCTATCGCCATTGCACAGTTTAATGATGACAGCCCGGA
AGCGAGGAAAATAACCCGGCGCTGGAGAATAGGTGAAGCAGCGGATTTAGTTGGGGTTTCTTCTC
AGGCTATCAGAGATGCCGAGAAAGCAGGGCGACTACCGCACCCGGATATGGAAATTCGAGGACG
GGTTGAGCAACGTGTTGGTTATACAATTGAACAAATTAATCATATGCGTGATGTGTTTGGTACGCG
ATTGCGACGTGCTGAAGACGTATTTCCACCGGTGATCGGGGTTGCTGCCCATAAAGGTGGCGTTTA
CAAAACCTCAGTTTCTGTTCATCTTGCTCAGGATCTGGCTCTGAAGGGGCTACGTGTTTTGCTCGTG
GAAGGTAACGACCCCCAGGGAACAGCCTCAATGTATCACGGATGGGTACCAGATCTTCATATTCA
TGCAGAAGACACTCTCCTGCCTTTCTATCTTGGGGAAAAGGACGATGTCACTTATGCAATAAAGCC
CACTTGCTGGCCGGGGCTTGACATTATTCCTTCCTGTCTGGCTCTGCACCGTATTGAAACTGAGTTA
ATGGGCAAATTTGATGAAGGTAAACTGCCCACCGATCCACACCTGATGCTCCGACTGGCCATTGAA
ACTGTTGCTCATGACTATGATGTCATAGTTATTGACAGCGCGCCTAACCTGGGTATCGGCACGATT
AATGTCGTATGTGCTGCTGATGTGCTGATTGTTCCCACGCCTGCTGAGTTGTTTGACTACACCTCCG
CACTGCAGTTTTTCGATATGCTTCGTGATCTGCTCAAGAACGTTGATCTTAAAGGGTTCGAGCCTG
ATGTACGTATTTTGCTTACCAAATACAGCAATAGTAATGGCTCTCAGTCCCCGTGGATGGAGGAGC
AAATTCGGGATGCCTGGGGAAGCATGGTTCTAAAAAATGTTGTACGTGAAACGGATGAAGTTGGT
AAAGGTCAGATCCGGATGAGAACTGTTTTTGAACAGGCCATTGATCAACGCTCTTCAACTGGTGCC
TGGAGAAATGCTCTTTCTATTTGGGAACCTGTCTGCAATGAAATTTTCGATCGTCTGATTAAACCAC
GCTGGGAGATTAGATAATGAAGCGTGCGCCTGTTATTCCAAAACATACGCTCAATACTCAACCGGT
TGAAGATACTTCGTTATCGACACCAGCTGCCCCGATGGTGGATTCGTTAATTGCGCGCGTAGGAGT
AATGGCTCGCGGTAATGCCATTACTTTGCCTGTATGTGGTCGGGATGTGAAGTTTACTCTTGAAGT
GCTCCGGGGTGATAGTGTTGAGAAGACCTCTCGGGTATGGTCAGGTAATGAACGTGACCAGGAGC
TGCTTACTGAGGACGCACTGGATGATCTCATCCCTTCTTTTCTACTGACTGGTCAACAGACACCGG
CGTTCGGTCGAAGAGTATCTGGTGTCATAGAAATTGCCGATGGGAGTCGCCGTCGTAAAGCTGCTG
CACTTACCGAAAGTGATTATCGTGTTCTGGTTGGCGAGCTGGATGATGAGCAGATGGCTGCATTAT
CCAGATTGGGTAACGATTATCGCCCAACAAGTGCTTATGAACGTGGTCAGCGTTATGCAAGCCGAT
TGCAGAATGAATTTGCTGGAAATATTTCTGCGCTGGCTGATGCGGAAAATATTTCACGTAAGATTA
TTACCCGCTGTATCAACACCGCCAAATTGCCTAAATCAGTTGTTGCTCTTTTTTCTCACCCCGGTGA
ACTATCTGCCCGGTCAGGTGATGCACTTCAAAAAGCCTTTACAGATAAAGAGGAATTACTTAAGCA
GCAGGCATCTAACCTTCATGAGCAGAAAAAAGCTGGGGTGATATTTGAAGCTGAAGAAGTTATCA
CTCTTTTAACTTCTGTGCTTAAAACGTCATCTGCATCAAGAACTAGTTTAAGCTCACGACATCAGTT
TGCTCCTGGAGCGACAGTATTGTATAAGGGCGATAAAATGGTGCTTAACCTGGACAGGTCTCGTGT
TCCAACTGAGTGTATAGAGAAAATTGAGGCCATTCTTAAGGAACTTGAAAAGCCAGCACCCTGAT
GCGACCACGTTTTAGTCTACGTTTATCTGTCTTTACTTAATGTCCTTTGTTACAGGCCAGAAAGCAT
AACTGGCCTGAATATTCTCTCTGGGCCCACTGTTCCACTTGTATCGTCGGTCTGATAATCAGACTGG
GACCACGGTCCCACTCGTATCGTCGGTCTGATTATTAGTCTGGGACCACGGTCCCACTCGTATCGT
CGGTCTGATTATTAGTCTGGGACCACGGTCCCACTCGTATCGTCGGTCTGATAATCAGACTGGGAC
CACGGTCCCACTCGTATCGTCGGTCTGATTATTAGTCTGGGACCATGGTCCCACTCGTATCGTCGGT
CTGATTATTAGTCTGGGACCACGGTCCCACTCGTATCGTCGGTCTGATTATTAGTCTGGAACCACG
GTCCCACTCGTATCGTCGGTCTGATTATTAGTCTGGGACCACGGTCCCACTCGTATCGTCGGTCTGA
TTATTAGTCTGGGACCACGATCCCACTCGTGTTGTCGGTCTGATTATCGGTCTGGGACCACGGTCCC
ACTTGTATTGTCGATCAGACTATCAGCGTGAGACTACGATTCCATCAATGCCTGTCAAGGGCAAGT
ATTGACATGTCGTCGTAACCTGTAGAACGGAGTAACCTCGGTGTGCGGTTGTATGCCTGCTGTGGA
TTGCTGCTGTGTCCTGCTTATCCACAACATTTTGCGCACGGTTATGTGGACAAAATACCTGGTTACC
CAGGCCGTGCCGGCACGTTAACCGGGCACATTTCCCCGAAAAGTGCCACCTGACGTCTAAGAAAC
CATTATTATCATGACATTAACCTATAAAAATAGGCGTATCACGAGGCCCTTTCGTCTTCAAGAATT
GGATCCGAATTCCCGGGAGAGCTCGATATCGCATGCGGATTTAAATTAATTAA
* C1F
(SEQ ID NO: 87)
CATCATCAATAATATACCTTATTTTGGATTGAAGCCAATATGATAATGAGGGGGTGGAGTTTGTGA
CGTGGCGCGGGGCGTGGGAACGGGGCGGGTGACGTAGTAGTGTGGCGGAAGTGTGATGTTGCAAG
TGTGGCGGAACACATGTAAGCGACGGATGTGGCAAAAGTGACGTTTTTGGTGTGCGCCGGTGTAC
ACAGGAAGTGACAATTTTCGCGCGGTTTTAGGCGGATGTTGTAGTAAATTTGGGCGTAACCGAGTA
AGATTTGGCCATTTTCGCGGGAAAACTGAATAAGAGGAAGTGAAATCTGAATAATTTTGTGTTACT
CATAGCGCGTAATATTTGTCTAGGGCCGCGGGGACTTTGACCGTTTACGTGGAGACTCGCCCAGGT
GTTTTTCTCAGGTGTTTTCCGCGTTCCGGGTCAAAGTTGGCGTTTTATTATTATAGTCAGTCGAAGC
TTGGATCCGGTACCTCTAGAATTCTCGAGCGGCCGCTAGCGACATCGGATCTCCCGATCCCCTATG
GTGCACTCTCAGTACAATCTGCTCTGATGCCGCATAGTTAAGCCAGTATCTGCTCCCTGCTTGTGTG
TTGGAGGTCGCTGAGTAGTGCGCGAGCAAAATTTAAGCTACAACAAGGCAAGGCTTGACCGACAA
TTGCATGAAGAATCTGCTTAGGGTTAGGCGTTTTGCGCTGCTTCGCGATGTACGGGCCAGATATAC
GCGTTGACATTGATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACG
TCAATGGGTGGACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCTCTG
GCTAACTAGAGAACCCACTGCTTACTGGCTTATCGAAATTAATACGACTCACTATAGGGAGACCCA
AGCTGGCTAGTTAAGCTATCAACAAGTTTGTACAAAAAAGCAGGCTTTAAAGGAACCAATTCAGT
CGACTGGATCCGGTACCACCATGTTCCTGAACTGCTGCCCAGGTTGCTGTATGGAGCCTGAATTCA
CCATGGTGAGCAAGGGCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACCCT
GACCTGGGGCGTGCAGTGCTTCGCCCGCTACCCCGACCACATGAAGCAGCACGACTTCTTCAAGTC
CGCCATGCCCGAAGGCTACGTCCAGGAGCGCACCATCTTCTTCAAGGACGACGGCAACTACAAGA
CCCGCGCCGAGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACGCCATCAGCGACAACGTCTA
TATCACCGCCGACAAGCAGAAGAACGGCATCAAGGCCAACTTCAAGATCCGCCACAACATCGAGG
ACGGCAGCGTGCAGCTCGCCGACCACTACCAGCAGAACACCCCCATCGGCGACGGCCCCGTGCTG
CTGCCCGACAACCACTACCTGAGCACCCAGTCCAAGCTGAGCAAAGACCCCAACGAGAAGCGCGA
TCACATGGTCCTGCTGGAGTTCGTGACCGCCGCCGGGATCACTCTCGGCATGGACGAGCTGTACAA
GGTCGACTATCCGTACGACGTACCAGACTACGCATAACCGCGGCCGCACTCGAGATATCTAGACC
CAGCTTTCTTGTACAAAGTGGTTGATCTAGAGGGCCCGCGGTTCGAAGGTAAGCCTATCCCTAACC
CTCTCCTCGGTCTCGATTCTACGCGTACCGGTTAGTAATGAGTTTAAACGGGGGAGGCTAACTGAA
ACACGGAAGGAGACAATACCGGAAGGAACCCGCGCTATGACGGCAATAAAAAGACAGAATAAAA
CGCACGGGTGTTGGGTCGTTTGTTCATAAACGCGGGGTTCGGTCCCAGGGCTGGCACTCTGTCGAT
ACCCCACCGAGACCCCATTGGGGCCAATACGCCCGCGTTTCTTCCTTTTCCCCACCCCACCCCCCA
AGTTCGGGTGAAGGCCCAGGGCTCGCAGCCAACGTCGGGGCGGCAGGCCCTGCCATAGCAGATCC
GATTCGACAGATCACTGAAATGTGTGGGCGTGGCTTAAGGGTGGGAAAGAATATATAAGGTGGGG
GTCTTATGTAGTTTTGTATCTGTTTTGCAGCAGCCGCCGCCGCCATGAGCACCAACTCGTTTGATGG
AAGCATTGTGAGCTCATATTTGACAACGCGCATGCCCCCATGGGCCGGGGTGCGTCAGAATGTGAT
GGGCTCCAGCATTGATGGTCGCCCCGTCCTGCCCGCAAACTCTACTACCTTGACCTACGAGACCGT
GTCTGGAACGCCGTTGGAGACTGCAGCCTCCGCCGCCGCTTCAGCCGCTGCAGCCACCGCCCGCGG
GATTGTGACTGACTTTGCTTTCCTGAGCCCGCTTGCAAGCAGTGCAGCTTCCCGTTCATCCGCCCGC
GATGACAAGTTGACGGCTCTTTTGGCACAATTGGATTCTTTGACCCGGGAACTTAATGTCGTTTCTC
AGCAGCTGTTGGATCTGCGCCAGCAGGTTTCTGCCCTGAAGGCTTCCTCCCCTCCCAATGCGGTTT
AAAACATAAATAAAAAACCAGACTCTGTTTGGATTTGGATCAAGCAAGTGTCTTGCTGTCTTTATT
TAGGGGTTTTGCGCGCGCGGTAGGCCCGGGACCAGCGGTCTCGGTCGTTGAGGGTCCTGTGTATTT
TTTCCAGGACGTGGTAAAGGTGACTCTGGATGTTCAGATACATGGGCATAAGCCCGTCTCTGGGGT
GGAGGTAGCACCACTGCAGAGCTTCATGCTGCGGGGTGGTGTTGTAGATGATCCAGTCGTAGCAG
GAGCGCTGGGCGTGGTGCCTAAAAATGTCTTTCAGTAGCAAGCTGATTGCCAGGGGCAGGCCCTT
GGTGTAAGTGTTTACAAAGCGGTTAAGCTGGGATGGGTGCATACGTGGGGATATGAGATGCATCT
TGGACTGTATTTTTAGGTTGGCTATGTTCCCAGCCATATCCCTCCGGGGATTCATGTTGTGCAGAAC
CACCAGCACAGTGTATCCGGTGCACTTGGGAAATTTGTCATGTAGCTTAGAAGGAAATGCGTGGA
AGAACTTGGAGACGCCCTTGTGACCTCCAAGATTTTCCATGCATTCGTCCATAATGATGGCAATGG
GCCCACGGGCGGCGGCCTGGGCGAAGATATTTCTGGGATCACTAACGTCATAGTTGTGTTCCAGGA
TGAGATCGTCATAGGCCATTTTTACAAAGCGCGGGCGGAGGGTGCCAGACTGCGGTATAATGGTT
CCATCCGGCCCAGGGGCGTAGTTACCCTCACAGATTTGCATTTCCCACGCTTTGAGTTCAGATGGG
GGGATCATGTCTACCTGCGGGGCGATGAAGAAAACGGTTTCCGGGGTAGGGGAGATCAGCTGGGA
AGAAAGCAGGTTCCTGAGCAGCTGCGACTTACCGCAGCCGGTGGGCCCGTAAATCACACCTATTA
CCGGCTGCAACTGGTAGTTAAGAGAGCTGCAGCTGCCGTCATCCCTGAGCAGGGGGGCCACTTCG
TTAAGCATGTCCCTGACTCGCATGTTTTCCCTGACCAAATCCGCCAGAAGGCGCTCGCCGCCCAGC
GATAGCAGTTCTTGCAAGGAAGCAAAGTTTTTCAACGGTTTGAGACCGTCCGCCGTAGGCATGCTT
TTGAGCGTTTGACCAAGCAGTTCCAGGCGGTCCCACAGCTCGGTCACCTGCTCTACGGCATCTCGA
TCCAGCATATCTCCTCGTTTCGCGGGTTGGGGCGGCTTTCGCTGTACGGCAGTAGTCGGTGCTCGTC
CAGACGGGCCAGGGTCATGTCTTTCCACGGGCGCAGGGTCCTCGTCAGCGTAGTCTGGGTCACGGT
GAAGGGGTGCGCTCCGGGCTGCGCGCTGGCCAGGGTGCGCTTGAGGCTGGTCCTGCTGGTGCTGA
AGCGCTGCCGGTCTTCGCCCTGCGCGTCGGCCAGGTAGCATTTGACCATGGTGTCATAGTCCAGCC
CCTCCGCGGCGTGGCCCTTGGCGCGCAGCTTGCCCTTGGAGGAGGCGCCGCACGAGGGGCAGTGC
AGACTTTTGAGGGCGTAGAGCTTGGGCGCGAGAAATACCGATTCCGGGGAGTAGGCATCCGCGCC
GCAGGCCCCGCAGACGGTCTCGCATTCCACGAGCCAGGTGAGCTCTGGCCGTTCGGGGTCAAAAA
CCAGGTTTCCCCCATGCTTTTTGATGCGTTTCTTACCTCTGGTTTCCATGAGCCGGTGTCCACGCTC
GGTGACGAAAAGGCTGTCCGTGTCCCCGTATACAGACTTGAGAGGCCTGTCCTCGAGCGGTGTTCC
GCGGTCCTCCTCGTATAGAAACTCGGACCACTCTGAGACAAAGGCTCGCGTCCAGGCCAGCACGA
AGGAGGCTAAGTGGGAGGGGTAGCGGTCGTTGTCCACTAGGGGGTCCACTCGCTCCAGGGTGTGA
AGACACATGTCGCCCTCTTCGGCATCAAGGAAGGTGATTGGTTTGTAGGTGTAGGCCACGTGACCG
GGTGTTCCTGAAGGGGGGCTATAAAAGGGGGTGGGGGCGCGTTCGTCCTCACTCTCTTCCGCATCG
CTGTCTGCGAGGGCCAGCTGTTGGGGTGAGTACTCCCTCTGAAAAGCGGGCATGACTTCTGCGCTA
AGATTGTCAGTTTCCAAAAACGAGGAGGATTTGATATTCACCTGGCCCGCGGTGATGCCTTTGAGG
GTGGCCGCATCCATCTGGTCAGAAAAGACAATCTTTTTGTTGTCAAGCTTGGTGGCAAACGACCCG
TAGAGGGCGTTGGACAGCAACTTGGCGATGGAGCGCAGGGTTTGGTTTTTGTCGCGATCGGCGCG
CTCCTTGGCCGCGATGTTTAGCTGCACGTATTCGCGCGCAACGCACCGCCATTCGGGAAAGACGGT
GGTGCGCTCGTCGGGCACCAGGTGCACGCGCCAACCGCGGTTGTGCAGGGTGACAAGGTCAACGC
TGGTGGCTACCTCTCCGCGTAGGCGCTCGTTGGTCCAGCAGAGGCGGCCGCCCTTGCGCGAGCAGA
ATGGCGGTAGGGGGTCTAGCTGCGTCTCGTCCGGGGGGTCTGCGTCCACGGTAAAGACCCCGGGC
AGCAGGCGCGCGTCGAAGTAGTCTATCTTGCATCCTTGCAAGTCTAGCGCCTGCTGCCATGCGCGG
GCGGCAAGCGCGCGCTCGTATGGGTTGAGTGGGGGACCCCATGGCATGGGGTGGGTGAGCGCGGA
GGCGTACATGCCGCAAATGTCGTAAACGTAGAGGGGCTCTCTGAGTATTCCAAGATATGTAGGGT
AGCATCTTCCACCGCGGATGCTGGCGCGCACGTAATCGTATAGTTCGTGCGAGGGAGCGAGGAGG
TCGGGACCGAGGTTGCTACGGGCGGGCTGCTCTGCTCGGAAGACTATCTGCCTGAAGATGGCATGT
GAGTTGGATGATATGGTTGGACGCTGGAAGACGTTGAAGCTGGCGTCTGTGAGACCTACCGCGTC
ACGCACGAAGGAGGCGTAGGAGTCGCGCAGCTTGTTGACCAGCTCGGCGGTGACCTGCACGTCTA
GGGCGCAGTAGTCCAGGGTTTCCTTGATGATGTCATACTTATCCTGTCCCTTTTTTTTCCACAGCTC
GCGGTTGAGGACAAACTCTTCGCGGTCTTTCCAGTACTCTTGGATCGGAAACCCGTCGGCCTCCGA
ACGGTAAGAGCCTAGCATGTAGAACTGGTTGACGGCCTGGTAGGCGCAGCATCCCTTTTCTACGGG
TAGCGCGTATGCCTGCGCGGCCTTCCGGAGCGAGGTGTGGGTGAGCGCAAAGGTGTCCCTGACCA
TGACCAGCATGAAGGGCACGAGCTGCTTCCCAAAGGCCCCCATCCAAGTATAGGTCTCTACATCGT
AGGTGACAAAGAGACGCTCGGTGCGAGGATGCGAGCCGATCGGGAAGAACTGGATCTCCCGCCAC
CAATTGGAGGAGTGGCTATTGATGTGGTGAAAGTAGAAGTCCCTGCGACGGGCCGAACACTCGTG
CTGGCTTTTGTAAAAACGTGCGCAGTACTGGCAGCGGTGCACGGGCTGTACATCCTGCACGAGGTT
GACCTGACGACCGCGCACAAGGAAGCAGAGTGGGAATTTGAGCCCCTCGCCTGGCGGGTTTGGCT
GGTGGTCTTCTACTTCGGCTGCTTGTCCTTGACCGTCTGGCTGCTCGAGGGGAGTTACGGTGGATC
GGACCACCACGCCGCGCGAGCCCAAAGTCCAGATGTCCGCGCGCGGCGGTCGGAGCTTGATGACA
ACATCGCGCAGATGGGAGCTGTCCATGGTCTGGAGCTCCCGCGGCGTCAGGTCAGGCGGGAGCTC
CTGCAGGTTTACCTCGCATAGACGGGTCAGGGCGCGGGCTAGATCCAGGTGATACCTAATTTCCAG
GGGCTGGTTGGTGGCGGCGTCGATGGCTTGCAAGAGGCCGCATCCCCGCGGCGCGACTACGGTAC
CGCGCGGCGGGCGGTGGGCCGCGGGGGTGTCCTTGGATGATGCATCTAAAAGCGGTGACGCGGGC
GAGCCCCCGGAGGTAGGGGGGGCTCCGGACCCGCCGGGAGAGGGGGCAGGGGCACGTCGGCGCC
GCGCGCGGGCAGGAGCTGGTGCTGCGCGCGTAGGTTGCTGGCGAACGCGACGACGCGGCGGTTGA
TCTCCTGAATCTGGCGCCTCTGCGTGAAGACGACGGGCCCGGTGAGCTTGAACCTGAAAGAGAGT
TCGACAGAATCAATTTCGGTGTCGTTGACGGCGGCCTGGCGCAAAATCTCCTGCACGTCTCCTGAG
TTGTCTTGATAGGCGATCTCGGCCATGAACTGCTCGATCTCTTCCTCCTGGAGATCTCCGCGTCCGG
CTCGCTCCACGGTGGCGGCGAGGTCGTTGGAAATGCGGGCCATGAGCTGCGAGAAGGCGTTGAGG
CCTCCCTCGTTCCAGACGCGGCTGTAGACCACGCCCCCTTCGGCATCGCGGGCGCGCATGACCACC
TGCGCGAGATTGAGCTCCACGTGCCGGGCGAAGACGGCGTAGTTTCGCAGGCGCTGAAAGAGGTA
GTTGAGGGTGGTGGCGGTGTGTTCTGCCACGAAGAAGTACATAACCCAGCGTCGCAACGTGGATT
CGTTGATATCCCCCAAGGCCTCAAGGCGCTCCATGGCCTCGTAGAAGTCCACGGCGAAGTTGAAA
AACTGGGAGTTGCGCGCCGACACGGTTAACTCCTCCTCCAGAAGACGGATGAGCTCGGCGACAGT
GTCGCGCACCTCGCGCTCAAAGGCTACAGGGGCCTCTTCTTCTTCTTCAATCTCCTCTTCCATAAGG
GCCTCCCCTTCTTCTTCTTCTGGCGGCGGTGGGGGAGGGGGGACACGGCGGCGACGACGGCGCAC
CGGGAGGCGGTCGACAAAGCGCTCGATCATCTCCCCGCGGCGACGGCGCATGGTCTCGGTGACGG
CGCGGCCGTTCTCGCGGGGGCGCAGTTGGAAGACGCCGCCCGTCATGTCCCGGTTATGGGTTGGCG
GGGGGCTGCCATGCGGCAGGGATACGGCGCTAACGATGCATCTCAACAATTGTTGTGTAGGTACT
CCGCCGCCGAGGGACCTGAGCGAGTCCGCATCGACCGGATCGGAAAACCTCTCGAGAAAGGCGTC
TAACCAGTCACAGTCGCAAGGTAGGCTGAGCACCGTGGCGGGCGGCAGCGGGCGGCGGTCGGGGT
TGTTTCTGGCGGAGGTGCTGCTGATGATGTAATTAAAGTAGGCGGTCTTGAGACGGCGGATGGTCG
ACAGAAGCACCATGTCCTTGGGTCCGGCCTGCTGAATGCGCAGGCGGTCGGCCATGCCCCAGGCTT
CGTTTTGACATCGGCGCAGGTCTTTGTAGTAGTCTTGCATGAGCCTTTCTACCGGCACTTCTTCTTC
TCCTTCCTCTTGTCCTGCATCTCTTGCATCTATCGCTGCGGCGGCGGCGGAGTTTGGCCGTAGGTGG
CGCCCTCTTCCTCCCATGCGTGTGACCCCGAAGCCCCTCATCGGCTGAAGCAGGGCTAGGTCGGCG
ACAACGCGCTCGGCTAATATGGCCTGCTGCACCTGCGTGAGGGTAGACTGGAAGTCATCCATGTCC
ACAAAGCGGTGGTATGCGCCCGTGTTGATGGTGTAAGTGCAGTTGGCCATAACGGACCAGTTAAC
GGTCTGGTGACCCGGCTGCGAGAGCTCGGTGTACCTGAGACGCGAGTAAGCCCTCGAGTCAAATA
CGTAGTCGTTGCAAGTCCGCACCAGGTACTGGTATCCCACCAAAAAGTGCGGCGGCGGCTGGCGG
TAGAGGGGCCAGCGTAGGGTGGCCGGGGCTCCGGGGGCGAGATCTTCCAACATAAGGCGATGATA
TCCGTAGATGTACCTGGACATCCAGGTGATGCCGGCGGCGGTGGTGGAGGCGCGCGGAAAGTCGC
GGACGCGGTTCCAGATGTTGCGCAGCGGCAAAAAGTGCTCCATGGTCGGGACGCTCTGGCCGGTC
AGGCGCGCGCAATCGTTGACGCTCTAGACCGTGCAAAAGGAGAGCCTGTAAGCGGGCACTCTTCC
GTGGTCTGGTGGATAAATTCGCAAGGGTATCATGGCGGACGACCGGGGTTCGAGCCCCGTATCCG
GCCGTCCGCCGTGATCCATGCGGTTACCGCCCGCGTGTCGAACCCAGGTGTGCGACGTCAGACAAC
GGGGGAGTGCTCCTTTTGGCTTCCTTCCAGGCGCGGCGGCTGCTGCGCTAGCTTTTTTGGCCACTGG
CCGCGCGCAGCGTAAGCGGTTAGGCTGGAAAGCGAAAGCATTAAGTGGCTCGCTCCCTGTAGCCG
GAGGGTTATTTTCCAAGGGTTGAGTCGCGGGACCCCCGGTTCGAGTCTCGGACCGGCCGGACTGCG
GCGAACGGGGGTTTGCCTCCCCGTCATGCAAGACCCCGCTTGCAAATTCCTCCGGAAACAGGGAC
GAGCCCCTTTTTTGCTTTTCCCAGATGCATCCGGTGCTGCGGCAGATGCGCCCCCCTCCTCAGCAGC
GGCAAGAGCAAGAGCAGCGGCAGACATGCAGGGCACCCTCCCCTCCTCCTACCGCGTCAGGAGGG
GCGACATCCGCGGTTGACGCGGCAGCAGATGGTGATTACGAACCCCCGCGGCGCCGGGCCCGGCA
CTACCTGGACTTGGAGGAGGGCGAGGGCCTGGCGCGGCTAGGAGCGCCCTCTCCTGAGCGGCACC
CAAGGGTGCAGCTGAAGCGTGATACGCGTGAGGCGTACGTGCCGCGGCAGAACCTGTTTCGCGAC
CGCGAGGGAGAGGAGCCCGAGGAGATGCGGGATCGAAAGTTCCACGCAGGGCGCGAGCTGCGGC
ATGGCCTGAATCGCGAGCGGTTGCTGCGCGAGGAGGACTTTGAGCCCGACGCGCGAACCGGGATT
AGTCCCGCGCGCGCACACGTGGCGGCCGCCGACCTGGTAACCGCATACGAGCAGACGGTGAACCA
GGAGATTAACTTTCAAAAAAGCTTTAACAACCACGTGCGTACGCTTGTGGCGCGCGAGGAGGTGG
CTATAGGACTGATGCATCTGTGGGACTTTGTAAGCGCGCTGGAGCAAAACCCAAATAGCAAGCCG
CTCATGGCGCAGCTGTTCCTTATAGTGCAGCACAGCAGGGACAACGAGGCATTCAGGGATGCGCT
GCTAAACATAGTAGAGCCCGAGGGCCGCTGGCTGCTCGATTTGATAAACATCCTGCAGAGCATAG
TGGTGCAGGAGCGCAGCTTGAGCCTGGCTGACAAGGTGGCCGCCATCAACTATTCCATGCTTAGCC
TGGGCAAGTTTTACGCCCGCAAGATATACCATACCCCTTACGTTCCCATAGACAAGGAGGTAAAG
ATCGAGGGGTTCTACATGCGCATGGCGCTGAAGGTGCTTACCTTGAGCGACGACCTGGGCGTTTAT
CGCAACGAGCGCATCCACAAGGCCGTGAGCGTGAGCCGGCGGCGCGAGCTCAGCGACCGCGAGCT
GATGCACAGCCTGCAAAGGGCCCTGGCTGGCACGGGCAGCGGCGATAGAGAGGCCGAGTCCTACT
TTGACGCGGGCGCTGACCTGCGCTGGGCCCCAAGCCGACGCGCCCTGGAGGCAGCTGGGGCCGGA
CCTGGGCTGGCGGTGGCACCCGCGCGCGCTGGCAACGTCGGCGGCGTGGAGGAATATGACGAGGA
CGATGAGTACGAGCCAGAGGACGGCGAGTACTAAGCGGTGATGTTTCTGATCAGATGATGCAAGA
CGCAACGGACCCGGCGGTGCGGGCGGCGCTGCAGAGCCAGCCGTCCGGCCTTAACTCCACGGACG
ACTGGCGCCAGGTCATGGACCGCATCATGTCGCTGACTGCGCGCAATCCTGACGCGTTCCGGCAGC
AGCCGCAGGCCAACCGGCTCTCCGCAATTCTGGAAGCGGTGGTCCCGGCGCGCGCAAACCCCACG
CACGAGAAGGTGCTGGCGATCGTAAACGCGCTGGCCGAAAACAGGGCCATCCGGCCCGACGAGG
CCGGCCTGGTCTACGACGCGCTGCTTCAGCGCGTGGCTCGTTACAACAGCGGCAACGTGCAGACC
AACCTGGACCGGCTGGTGGGGGATGTGCGCGAGGCCGTGGCGCAGCGTGAGCGCGCGCAGCAGC
AGGGCAACCTGGGCTCCATGGTTGCACTAAACGCCTTCCTGAGTACACAGCCCGCCAACGTGCCGC
GGGGACAGGAGGACTACACCAACTTTGTGAGCGCACTGCGGCTAATGGTGACTGAGACACCGCAA
AGTGAGGTGTACCAGTCTGGGCCAGACTATTTTTTCCAGACCAGTAGACAAGGCCTGCAGACCGTA
AACCTGAGCCAGGCTTTCAAAAACTTGCAGGGGCTGTGGGGGGTGCGGGCTCCCACAGGCGACCG
CGCGACCGTGTCTAGCTTGCTGACGCCCAACTCGCGCCTGTTGCTGCTGCTAATAGCGCCCTTCAC
GGACAGTGGCAGCGTGTCCCGGGACACATACCTAGGTCACTTGCTGACACTGTACCGCGAGGCCA
TAGGTCAGGCGCATGTGGACGAGCATACTTTCCAGGAGATTACAAGTGTCAGCCGCGCGCTGGGG
CAGGAGGACACGGGCAGCCTGGAGGCAACCCTAAACTACCTGCTGACCAACCGGCGGCAGAAGA
TCCCCTCGTTGCACAGTTTAAACAGCGAGGAGGAGCGCATTTTGCGCTACGTGCAGCAGAGCGTG
AGCCTTAACCTGATGCGCGACGGGGTAACGCCCAGCGTGGCGCTGGACATGACCGCGCGCAACAT
GGAACCGGGCATGTATGCCTCAAACCGGCCGTTTATCAACCGCCTAATGGACTACTTGCATCGCGC
GGCCGCCGTGAACCCCGAGTATTTCACCAATGCCATCTTGAACCCGCACTGGCTACCGCCCCCTGG
TTTCTACACCGGGGGATTCGAGGTGCCCGAGGGTAACGATGGATTCCTCTGGGACGACATAGACG
ACAGCGTGTTTTCCCCGCAACCGCAGACCCTGCTAGAGTTGCAACAGCGCGAGCAGGCAGAGGCG
GCGCTGCGAAAGGAAAGCTTCCGCAGGCCAAGCAGCTTGTCCGATCTAGGCGCTGCGGCCCCGCG
GTCAGATGCTAGTAGCCCATTTCCAAGCTTGATAGGGTCTCTTACCAGCACTCGCACCACCCGCCC
GCGCCTGCTGGGCGAGGAGGAGTACCTAAACAACTCGCTGCTGCAGCCGCAGCGCGAAAAAAACC
TGCCTCCGGCATTTCCCAACAACGGGATAGAGAGCCTAGTGGACAAGATGAGTAGATGGAAGACG
TACGCGCAGGAGCACAGGGACGTGCCAGGCCCGCGCCCGCCCACCCGTCGTCAAAGGCACGACCG
TCAGCGGGGTCTGGTGTGGGAGGACGATGACTCGGCAGACGACAGCAGCGTCCTGGATTTGGGAG
GGAGTGGCAACCCGTTTGCGCACCTTCGCCCCAGGCTGGGGAGAATGTTTTAAAAAAAAAAAAAG
CATGATGCAAAATAAAAAACTCACCAAGGCCATGGCACCGAGCGTTGGTTTTCTTGTATTCCCCTT
AGTATGCGGCGCGCGGCGATGTATGAGGAAGGTCCTCCTCCCTCCTACGAGAGTGTGGTGAGCGC
GGCGCCAGTGGCGGCGGCGCTGGGTTCTCCCTTCGATGCTCCCCTGGACCCGCCGTTTGTGCCTCC
GCGGTACCTGCGGCCTACCGGGGGGAGAAACAGCATCCGTTACTCTGAGTTGGCACCCCTATTCGA
CACCACCCGTGTGTACCTGGTGGACAACAAGTCAACGGATGTGGCATCCCTGAACTACCAGAACG
ACCACAGCAACTTTCTGACCACGGTCATTCAAAACAATGACTACAGCCCGGGGGAGGCAAGCACA
CAGACCATCAATCTTGACGACCGGTCGCACTGGGGCGGCGACCTGAAAACCATCCTGCATACCAA
CATGCCAAATGTGAACGAGTTCATGTTTACCAATAAGTTTAAGGCGCGGGTGATGGTGTCGCGCTT
GCCTACTAAGGACAATCAGGTGGAGCTGAAATACGAGTGGGTGGAGTTCACGCTGCCCGAGGGCA
ACTACTCCGAGACCATGACCATAGACCTTATGAACAACGCGATCGTGGAGCACTACTTGAAAGTG
GGCAGACAGAACGGGGTTCTGGAAAGCGACATCGGGGTAAAGTTTGACACCCGCAACTTCAGACT
GGGGTTTGACCCCGTCACTGGTCTTGTCATGCCTGGGGTATATACAAACGAAGCCTTCCATCCAGA
CATCATTTTGCTGCCAGGATGCGGGGTGGACTTCACCCACAGCCGCCTGAGCAACTTGTTGGGCAT
CCGCAAGCGGCAACCCTTCCAGGAGGGCTTTAGGATCACCTACGATGATCTGGAGGGTGGTAACA
TTCCCGCACTGTTGGATGTGGACGCCTACCAGGCGAGCTTGAAAGATGACACCGAACAGGGCGGG
GGTGGCGCAGGCGGCAGCAACAGCAGTGGCAGCGGCGCGGAAGAGAACTCCAACGCGGCAGCCG
CGGCAATGCAGCCGGTGGAGGACATGAACGATCATGCCATTCGCGGCGACACCTTTGCCACACGG
GCTGAGGAGAAGCGCGCTGAGGCCGAAGCAGCGGCCGAAGCTGCCGCCCCCGCTGCGCAACCCG
AGGTCGAGAAGCCTCAGAAGAAACCGGTGATCAAACCCCTGACAGAGGACAGCAAGAAACGCAG
TTACAACCTAATAAGCAATGACAGCACCTTCACCCAGTACCGCAGCTGGTACCTTGCATACAACTA
CGGCGACCCTCAGACCGGAATCCGCTCATGGACCCTGCTTTGCACTCCTGACGTAACCTGCGGCTC
GGAGCAGGTCTACTGGTCGTTGCCAGACATGATGCAAGACCCCGTGACCTTCCGCTCCACGCGCCA
GATCAGCAACTTTCCGGTGGTGGGCGCCGAGCTGTTGCCCGTGCACTCCAAGAGCTTCTACAACGA
CCAGGCCGTCTACTCCCAACTCATCCGCCAGTTTACCTCTCTGACCCACGTGTTCAATCGCTTTCCC
GAGAACCAGATTTTGGCGCGCCCGCCAGCCCCCACCATCACCACCGTCAGTGAAAACGTTCCTGCT
CTCACAGATCACGGGACGCTACCGCTGCGCAACAGCATCGGAGGAGTCCAGCGAGTGACCATTAC
TGACGCCAGACGCCGCACCTGCCCCTACGTTTACAAGGCCCTGGGCATAGTCTCGCCGCGCGTCCT
ATCGAGCCGCACTTTTTGAGCAAGCATGTCCATCCTTATATCGCCCAGCAATAACACAGGCTGGGG
CCTGCGCTTCCCAAGCAAGATGTTTGGCGGGGCCAAGAAGCGCTCCGACCAACACCCAGTGCGCG
TGCGCGGGCACTACCGCGCGCCCTGGGGCGCGCACAAACGCGGCCGCACTGGGCGCACCACCGTC
GATGACGCCATCGACGCGGTGGTGGAGGAGGCGCGCAACTACACGCCCACGCCGCCACCAGTGTC
CACAGTGGACGCGGCCATTCAGACCGTGGTGCGCGGAGCCCGGCGCTATGCTAAAATGAAGAGAC
GGCGGAGGCGCGTAGCACGTCGCCACCGCCGCCGACCCGGCACTGCCGCCCAACGCGCGGCGGCG
GCCCTGCTTAACCGCGCACGTCGCACCGGCCGACGGGCGGCCATGCGGGCCGCTCGAAGGCTGGC
CGCGGGTATTGTCACTGTGCCCCCCAGGTCCAGGCGACGAGCGGCCGCCGCAGCAGCCGCGGCCA
TTAGTGCTATGACTCAGGGTCGCAGGGGCAACGTGTATTGGGTGCGCGACTCGGTTAGCGGCCTGC
GCGTGCCCGTGCGCACCCGCCCCCCGCGCAACTAGATTGCAAGAAAAAACTACTTAGACTCGTACT
GTTGTATGTATCCAGCGGCGGCGGCGCGCAACGAAGCTATGTCCAAGCGCAAAATCAAAGAAGAG
ATGCTCCAGGTCATCGCGCCGGAGATCTATGGCCCCCCGAAGAAGGAAGAGCAGGATTACAAGCC
CCGAAAGCTAAAGCGGGTCAAAAAGAAAAAGAAAGATGATGATGATGAACTTGACGACGAGGTG
GAACTGCTGCACGCTACCGCGCCCAGGCGACGGGTACAGTGGAAAGGTCGACGCGTAAAACGTGT
TTTGCGACCCGGCACCACCGTAGTCTTTACGCCCGGTGAGCGCTCCACCCGCACCTACAAGCGCGT
GTATGATGAGGTGTACGGCGACGAGGACCTGCTTGAGCAGGCCAACGAGCGCCTCGGGGAGTTTG
CCTACGGAAAGCGGCATAAGGACATGCTGGCGTTGCCGCTGGACGAGGGCAACCCAACACCTAGC
CTAAAGCCCGTAACACTGCAGCAGGTGCTGCCCGCGCTTGCACCGTCCGAAGAAAAGCGCGGCCT
AAAGCGCGAGTCTGGTGACTTGGCACCCACCGTGCAGCTGATGGTACCCAAGCGCCAGCGACTGG
AAGATGTCTTGGAAAAAATGACCGTGGAACCTGGGCTGGAGCCCGAGGTCCGCGTGCGGCCAATC
AAGCAGGTGGCGCCGGGACTGGGCGTGCAGACCGTGGACGTTCAGATACCCACTACCAGTAGCAC
CAGTATTGCCACCGCCACAGAGGGCATGGAGACACAAACGTCCCCGGTTGCCTCAGCGGTGGCGG
ATGCCGCGGTGCAGGCGGTCGCTGCGGCCGCGTCCAAGACCTCTACGGAGGTGCAAACGGACCCG
TGGATGTTTCGCGTTTCAGCCCCCCGGCGCCCGCGCCGTTCGAGGAAGTACGGCGCCGCCAGCGCG
CTACTGCCCGAATATGCCCTACATCCTTCCATTGCGCCTACCCCCGGCTATCGTGGCTACACCTACC
GCCCCAGAAGACGAGCAACTACCCGACGCCGAACCACCACTGGAACCCGCCGCCGCCGTCGCCGT
CGCCAGCCCGTGCTGGCCCCGATTTCCGTGCGCAGGGTGGCTCGCGAAGGAGGCAGGACCCTGGT
GCTGCCAACAGCGCGCTACCACCCCAGCATCGTTTAAAAGCCGGTCTTTGTGGTTCTTGCAGATAT
GGCCCTCACCTGCCGCCTCCGTTTCCCGGTGCCGGGATTCCGAGGAAGAATGCACCGTAGGAGGG
GCATGGCCGGCCACGGCCTGACGGGCGGCATGCGTCGTGCGCACCACCGGCGGCGGCGCGCGTCG
CACCGTCGCATGCGCGGCGGTATCCTGCCCCTCCTTATTCCACTGATCGCCGCGGCGATTGGCGCC
GTGCCCGGAATTGCATCCGTGGCCTTGCAGGCGCAGAGACACTGATTAAAAACAAGTTGCATGTG
GAAAAATCAAAATAAAAAGTCTGGACTCTCACGCTCGCTTGGTCCTGTAACTATTTTGTAGAATGG
AAGACATCAACTTTGCGTCTCTGGCCCCGCGACACGGCTCGCGCCCGTTCATGGGAAACTGGCAAG
ATATCGGCACCAGCAATATGAGCGGTGGCGCCTTCAGCTGGGGCTCGCTGTGGAGCGGCATTAAA
AATTTCGGTTCCACCGTTAAGAACTATGGCAGCAAGGCCTGGAACAGCAGCACAGGCCAGATGCT
GAGGGATAAGTTGAAAGAGCAAAATTTCCAACAAAAGGTGGTAGATGGCCTGGCCTCTGGCATTA
GCGGGGTGGTGGACCTGGCCAACCAGGCAGTGCAAAATAAGATTAACAGTAAGCTTGATCCCCGC
CCTCCCGTAGAGGAGCCTCCACCGGCCGTGGAGACAGTGTCTCCAGAGGGGCGTGGCGAAAAGCG
TCCGCGCCCCGACAGGGAAGAAACTCTGGTGACGCAAATAGACGAGCCTCCCTCGTACGAGGAGG
CACTAAAGCAAGGCCTGCCCACCACCCGTCCCATCGCGCCCATGGCTACCGGAGTGCTGGGCCAG
CACACACCCGTAACGCTGGACCTGCCTCCCCCCGCCGACACCCAGCAGAAACCTGTGCTGCCAGG
CCCGACCGCCGTTGTTGTAACCCGTCCTAGCCGCGCGTCCCTGCGCCGCGCCGCCAGCGGTCCGCG
ATCGTTGCGGCCCGTAGCCAGTGGCAACTGGCAAAGCACACTGAACAGCATCGTGGGTCTGGGGG
TGCAATCCCTGAAGCGCCGACGATGCTTCTGATAGCTAACGTGTCGTATGTGTGTCATGTATGCGT
CCATGTCGCCGCCAGAGGAGCTGCTGAGCCGCCGCGCGCCCGCTTTCCAAGATGGCTACCCCTTCG
ATGATGCCGCAGTGGTCTTACATGCACATCTCGGGCCAGGACGCCTCGGAGTACCTGAGCCCCGG
GCTGGTGCAGTTTGCCCGCGCCACCGAGACGTACTTCAGCCTGAATAACAAGTTTAGAAACCCCAC
GGTGGCGCCTACGCACGACGTGACCACAGACCGGTCCCAGCGTTTGACGCTGCGGTTCATCCCTGT
GGACCGTGAGGATACTGCGTACTCGTACAAGGCGCGGTTCACCCTAGCTGTGGGTGATAACCGTGT
GCTGGACATGGCTTCCACGTACTTTGACATCCGCGGCGTGCTGGACAGGGGCCCTACTTTTAAGCC
CTACTCTGGCACTGCCTACAACGCCCTGGCTCCCAAGGGTGCCCCAAATCCTTGCGAATGGGATGA
AGCTGCTACTGCTCTTGAAATAAACCTAGAAGAAGAGGACGATGACAACGAAGACGAAGTAGAC
GAGCAAGCTGAGCAGCAAAAAACTCACGTATTTGGGCAGGCGCCTTATTCTGGTATAAATATTAC
AAAGGAGGGTATTCAAATAGGTGTCGAAGGTCAAACACCTAAATATGCCGATAAAACATTTCAAC
CTGAACCTCAAATAGGAGAATCTCAGTGGTACGAAACAGAAATTAATCATGCAGCTGGGAGAGTC
CTAAAAAAGACTACCCCAATGAAACCATGTTACGGTTCATATGCAAAACCCACAAATGAAAATGG
AGGGCAAGGCATTCTTGTAAAGCAACAAAATGGAAAGCTAGAAAGTCAAGTGGAAATGCAATTTT
TCTCAACTACTGAGGCAGCCGCAGGCAATGGTGATAACTTGACTCCTAAAGTGGTATTGTACAGTG
AAGATGTAGATATAGAAACCCCAGACACTCATATTTCTTACATGCCCACTATTAAGGAAGGTAACT
CACGAGAACTAATGGGCCAACAATCTATGCCCAACAGGCCTAATTACATTGCTTTTAGGGACAATT
TTATTGGTCTAATGTATTACAACAGCACGGGTAATATGGGTGTTCTGGCGGGCCAAGCATCGCAGT
TGAATGCTGTTGTAGATTTGCAAGACAGAAACACAGAGCTTTCATACCAGCTTTTGCTTGATTCCA
TTGGTGATAGAACCAGGTACTTTTCTATGTGGAATCAGGCTGTTGACAGCTATGATCCAGATGTTA
GAATTATTGAAAATCATGGAACTGAAGATGAACTTCCAAATTACTGCTTTCCACTGGGAGGTGTGA
TTAATACAGAGACTCTTACCAAGGTAAAACCTAAAACAGGTCAGGAAAATGGATGGGAAAAAGAT
GCTACAGAATTTTCAGATAAAAATGAAATAAGAGTTGGAAATAATTTTGCCATGGAAATCAATCT
AAATGCCAACCTGTGGAGAAATTTCCTGTACTCCAACATAGCGCTGTATTTGCCCGACAAGCTAAA
GTACAGTCCTTCCAACGTAAAAATTTCTGATAACCCAAACACCTACGACTACATGAACAAGCGAGT
GGTGGCTCCCGGGCTAGTGGACTGCTACATTAACCTTGGAGCACGCTGGTCCCTTGACTATATGGA
CAACGTCAACCCATTTAACCACCACCGCAATGCTGGCCTGCGCTACCGCTCAATGTTGCTGGGCAA
TGGTCGCTATGTGCCCTTCCACATCCAGGTGCCTCAGAAGTTCTTTGCCATTAAAAACCTCCTTCTC
CTGCCGGGCTCATACACCTACGAGTGGAACTTCAGGAAGGATGTTAACATGGTTCTGCAGAGCTCC
CTAGGAAATGACCTAAGGGTTGACGGAGCCAGCATTAAGTTTGATAGCATTTGCCTTTACGCCACC
TTCTTCCCCATGGCCCACAACACCGCCTCCACGCTTGAGGCCATGCTTAGAAACGACACCAACGAC
CAGTCCTTTAACGACTATCTCTCCGCCGCCAACATGCTCTACCCTATACCCGCCAACGCTACCAAC
GTGCCCATATCCATCCCCTCCCGCAACTGGGCGGCTTTCCGCGGCTGGGCCTTCACGCGCCTTAAG
ACTAAGGAAACCCCATCACTGGGCTCGGGCTACGACCCTTATTACACCTACTCTGGCTCTATACCC
TACCTAGATGGAACCTTTTACCTCAACCACACCTTTAAGAAGGTGGCCATTACCTTTGACTCTTCTG
TCAGCTGGCCTGGCAATGACCGCCTGCTTACCCCCAACGAGTTTGAAATTAAGCGCTCAGTTGACG
GGGAGGGTTACAACGTTGCCCAGTGTAACATGACCAAAGACTGGTTCCTGGTACAAATGCTAGCT
AACTATAACATTGGCTACCAGGGCTTCTATATCCCAGAGAGCTACAAGGACCGCATGTACTCCTTC
TTTAGAAACTTCCAGCCCATGAGCCGTCAGGTGGTGGATGATACTAAATACAAGGACTACCAACA
GGTGGGCATCCTACACCAACACAACAACTCTGGATTTGTTGGCTACCTTGCCCCCACCATGCGCGA
AGGACAGGCCTACCCTGCTAACTTCCCCTATCCGCTTATAGGCAAGACCGCAGTTGACAGCATTAC
CCAGAAAAAGTTTCTTTGCGATCGCACCCTTTGGCGCATCCCATTCTCCAGTAACTTTATGTCCATG
GGCGCACTCACAGACCTGGGCCAAAACCTTCTCTACGCCAACTCCGCCCACGCGCTAGACATGACT
TTTGAGGTGGATCCCATGGACGAGCCCACCCTTCTTTATGTTTTGTTTGAAGTCTTTGACGTGGTCC
GTGTGCACCAGCCGCACCGCGGCGTCATCGAAACCGTGTACCTGCGCACGCCCTTCTCGGCCGGCA
ACGCCACAACATAAAGAAGCAAGCAACATCAACAACAGCTGCCGCCATGGGCTCCAGTGAGCAG
GAACTGAAAGCCATTGTCAAAGATCTTGGTTGTGGGCCATATTTTTTGGGCACCTATGACAAGCGC
TTTCCAGGCTTTGTTTCTCCACACAAGCTCGCCTGCGCCATAGTCAATACGGCCGGTCGCGAGACT
GGGGGCGTACACTGGATGGCCTTTGCCTGGAACCCGCACTCAAAAACATGCTACCTCTTTGAGCCC
TTTGGCTTTTCTGACCAGCGACTCAAGCAGGTTTACCAGTTTGAGTACGAGTCACTCCTGCGCCGT
AGCGCCATTGCTTCTTCCCCCGACCGCTGTATAACGCTGGAAAAGTCCACCCAAAGCGTACAGGGG
CCCAACTCGGCCGCCTGTGGACTATTCTGCTGCATGTTTCTCCACGCCTTTGCCAACTGGCCCCAAA
CTCCCATGGATCACAACCCCACCATGAACCTTATTACCGGGGTACCCAACTCCATGCTCAACAGTC
CCCAGGTACAGCCCACCCTGCGTCGCAACCAGGAACAGCTCTACAGCTTCCTGGAGCGCCACTCGC
CCTACTTCCGCAGCCACAGTGCGCAGATTAGGAGCGCCACTTCTTTTTGTCACTTGAAAAACATGT
AAAAATAATGTACTAGAGACACTTTCAATAAAGGCAAATGCTTTTATTTGTACACTCTCGGGTGAT
TATTTACCCCCACCCTTGCCGTCTGCGCCGTTTAAAAATCAAAGGGGTTCTGCCGCGCATCGCTAT
GCGCCACTGGCAGGGACACGTTGCGATACTGGTGTTTAGTGCTCCACTTAAACTCAGGCACAACCA
TCCGCGGCAGCTCGGTGAAGTTTTCACTCCACAGGCTGCGCACCATCACCAACGCGTTTAGCAGGT
CGGGCGCCGATATCTTGAAGTCGCAGTTGGGGCCTCCGCCCTGCGCGCGCGAGTTGCGATACACA
GGGTTGCAGCACTGGAACACTATCAGCGCCGGGTGGTGCACGCTGGCCAGCACGCTCTTGTCGGA
GATCAGATCCGCGTCCAGGTCCTCCGCGTTGCTCAGGGCGAACGGAGTCAACTTTGGTAGCTGCCT
TCCCAAAAAGGGCGCGTGCCCAGGCTTTGAGTTGCACTCGCACCGTAGTGGCATCAAAAGGTGAC
CGTGCCCGGTCTGGGCGTTAGGATACAGCGCCTGCATAAAAGCCTTGATCTGCTTAAAAGCCACCT
GAGCCTTTGCGCCTTCAGAGAAGAACATGCCGCAAGACTTGCCGGAAAACTGATTGGCCGGACAG
GCCGCGTCGTGCACGCAGCACCTTGCGTCGGTGTTGGAGATCTGCACCACATTTCGGCCCCACCGG
TTCTTCACGATCTTGGCCTTGCTAGACTGCTCCTTCAGCGCGCGCTGCCCGTTTTCGCTCGTCACAT
CCATTTCAATCACGTGCTCCTTATTTATCATAATGCTTCCGTGTAGACACTTAAGCTCGCCTTCGAT
CTCAGCGCAGCGGTGCAGCCACAACGCGCAGCCCGTGGGCTCGTGATGCTTGTAGGTCACCTCTGC
AAACGACTGCAGGTACGCCTGCAGGAATCGCCCCATCATCGTCACAAAGGTCTTGTTGCTGGTGAA
GGTCAGCTGCAACCCGCGGTGCTCCTCGTTCAGCCAGGTCTTGCATACGGCCGCCAGAGCTTCCAC
TTGGTCAGGCAGTAGTTTGAAGTTCGCCTTTAGATCGTTATCCACGTGGTACTTGTCCATCAGCGCG
CGCGCAGCCTCCATGCCCTTCTCCCACGCAGACACGATCGGCACACTCAGCGGGTTCATCACCGTA
ATTTCACTTTCCGCTTCGCTGGGCTCTTCCTCTTCCTCTTGCGTCCGCATACCACGCGCCACTGGGT
CGTCTTCATTCAGCCGCCGCACTGTGCGCTTACCTCCTTTGCCATGCTTGATTAGCACCGGTGGGTT
GCTGAAACCCACCATTTGTAGCGCCACATCTTCTCTTTCTTCCTCGCTGTCCACGATTACTTGACAA
TTAATCATCGGCTCGTATAATGATGCAGTACATTTTCACAGGAGGTACAGCTATGACCATGATTAC
GGATTCACTGGCCGTCGTTTTACAACGTCGTGACTGGGAAAACCCTGGCGTTACCCAACTTAATCG
CCTTGCAGCACATCCCCCTTTCGCCAGCTGGCGTAATAGCGAAGAGGCCCGCACCGATCGCCCTTC
CCAACAGTTGCGCAGCCTGAATGGCGAATAGGTCGCGCCGCACCGCGTCCGCGCTCGGGGGTGGT
TTCGCGCTGCTCCTCTTCCCGACTGGCCATTTCCTTCTCCTATAGGCAGAAAAAGATCATGGAGTCA
GTCGAGAAGAAGGACAGCCTAACCGCCCCCTCTGAGTTCGCCACCACCGCCTCCACCGATGCCGC
CAACGCGCCTACCACCTTCCCCGTCGAGGCACCCCCGCTTGAGGAGGAGGAAGTGATTATCGAGC
AGGACCCAGGTTTTGTAAGCGAAGACGACGAGGACCGCTCAGTACCAACAGAGGATAAAAAGCA
AGACCAGGACAACGCAGAGGCAAACGAGGAACAAGTCGGGCGGGGGGACGAAAGGCATGGCGA
CTACCTAGATGTGGGAGACGACGTGCTGTTGAAGCATCTGCAGCGCCAGTGCGCCATTATCTGCGA
CGCGTTGCAAGAGCGCAGCGATGTGCCCCTCGCCATAGCGGATGTCAGCCTTGCCTACGAACGCC
ACCTATTCTCACCGCGCGTACCCCCCAAACGCCAAGAAAACGGCACATGCGAGCCCAACCCGCGC
CTCAACTTCTACCCCGTATTTGCCGTGCCAGAGGTGCTTGCCACCTATCACATCTTTTTCCAAAACT
GCAAGATACCCCTATCCTGCCGTGCCAACCGCAGCCGAGCGGACAAGCAGCTGGCCTTGCGGCAG
GGCGCTGTCATACCTGATATCGCCTCGCTCAACGAAGTGCCAAAAATCTTTGAGGGTCTTGGACGC
GACGAGAAGCGCGCGGCAAACGCTCTGCAACAGGAAAACAGCGAAAATGAAAGTCACTCTGGAG
TGTTGGTGGAACTCGAGGGTGACAACGCGCGCCTAGCCGTACTAAAACGCAGCATCGAGGTCACC
CACTTTGCCTACCCGGCACTTAACCTACCCCCCAAGGTCATGAGCACAGTCATGAGTGAGCTGATC
GTGCGCCGTGCGCAGCCCCTGGAGAGGGATGCAAATTTGCAAGAACAAACAGAGGAGGGCCTACC
CGCAGTTGGCGACGAGCAGCTAGCGCGCTGGCTTCAAACGCGCGAGCCTGCCGACTTGGAGGAGC
GACGCAAACTAATGATGGCCGCAGTGCTCGTTACCGTGGAGCTTGAGTGCATGCAGCGGTTCTTTG
CTGACCCGGAGATGCAGCGCAAGCTAGAGGAAACATTGCACTACACCTTTCGACAGGGCTACGTA
CGCCAGGCCTGCAAGATCTCCAACGTGGAGCTCTGCAACCTGGTCTCCTACCTTGGAATTTTGCAC
GAAAACCGCCTTGGGCAAAACGTGCTTCATTCCACGCTCAAGGGCGAGGCGCGCCGCGACTACGT
CCGCGACTGCGTTTACTTATTTCTATGCTACACCTGGCAGACGGCCATGGGCGTTTGGCAGCAGTG
CTTGGAGGAGTGCAACCTCAAGGAGCTGCAGAAACTGCTAAAGCAAAACTTGAAGGACCTATGGA
CGGCCTTCAACGAGCGCTCCGTGGCCGCGCACCTGGCGGACATCATTTTCCCCGAACGCCTGCTTA
AAACCCTGCAACAGGGTCTGCCAGACTTCACCAGTCAAAGCATGTTGCAGAACTTTAGGAACTTTA
TCCTAGAGCGCTCAGGAATCTTGCCCGCCACCTGCTGTGCACTTCCTAGCGACTTTGTGCCCATTAA
GTACCGCGAATGCCCTCCGCCGCTTTGGGGCCACTGCTACCTTCTGCAGCTAGCCAACTACCTTGC
CTACCACTCTGACATAATGGAAGACGTGAGCGGTGACGGTCTACTGGAGTGTCACTGTCGCTGCAA
CCTATGCACCCCGCACCGCTCCCTGGTTTGCAATTCGCAGCTGCTTAACGAAAGTCAAATTATCGG
TACCTTTGAGCTGCAGGGTCCCTCGCCTGACGAAAAGTCCGCGGCTCCGGGGTTGAAACTCACTCC
GGGGCTGTGGACGTCGGCTTACCTTCGCAAATTTGTACCTGAGGACTACCACGCCCACGAGATTAG
GTTCTACGAAGACCAATCCCGCCCGCCTAATGCGGAGCTTACCGCCTGCGTCATTACCCAGGGCCA
CATTCTTGGCCAATTGCAAGCCATCAACAAAGCCCGCCAAGAGTTTCTGCTACGAAAGGGACGGG
GGGTTTACTTGGACCCCCAGTCCGGCGAGGAGCTCAACCCAATCCCCCCGCCGCCGCAGCCCTATC
AGCAGCAGCCGCGGGCCCTTGCTTCCCAGGATGGCACCCAAAAAGAAGCTGCAGCTGCCGCCGCC
ACCCACGGACGAGGAGGAATACTGGGACAGTCAGGCAGAGGAGGTTTTGGACGAGGAGGAGGAG
GACATGATGGAAGACTGGGAGAGCCTAGACGAGGAAGCTTCCGAGGTCGAAGAGGTGTCAGACG
AAACACCGTCACCCTCGGTCGCATTCCCCTCGCCGGCGCCCCAGAAATCGGCAACCGGTTCCAGCA
TGGCTACAACCTCCGCTCCTCAGGCGCCGCCGGCACTGCCCGTTCGCCGACCCAACCGTAGATGGG
ACACCACTGGAACCAGGGCCGGTAAGTCCAAGCAGCCGCCGCCGTTAGCCCAAGAGCAACAACAG
CGCCAAGGCTACCGCTCATGGCGCGGGCACAAGAACGCCATAGTTGCTTGCTTGCAAGACTGTGG
GGGCAACATCTCCTTCGCCCGCCGCTTTCTTCTCTACCATCACGGCGTGGCCTTCCCCCGTAACATC
CTGCATTACTACCGTCATCTCTACAGCCCATACTGCACCGGCGGCAGCGGCAGCAACAGCAGCGG
CCACACAGAAGCAAAGGCGACCGGATAGCAAGACTCTGACAAAGCCCAAGAAATCCACAGCGGC
GGCAGCAGCAGGAGGAGGAGCGCTGCGTCTGGCGCCCAACGAACCCGTATCGACCCGCGAGCTTA
GAAACAGGATTTTTCCCACTCTGTATGCTATATTTCAACAGAGCAGGGGCCAAGAACAAGAGCTG
AAAATAAAAAACAGGTCTCTGCGATCCCTCACCCGCAGCTGCCTGTATCACAAAAGCGAAGATCA
GCTTCGGCGCACGCTGGAAGACGCGGAGGCTCTCTTCAGTAAATACTGCGCGCTGACTCTTAAGGA
CTAGTTTCGCGCCCTTTCTCAAATTTAAGCGCGAAAACTACGTCATCTCCAGCGGCCACACCCGGC
GCCAGCACCTGTTGTCAGCGCCATTATGAGCAAGGAAATTCCCACGCCCTACATGTGGAGTTACCA
GCCACAAATGGGACTTGCGGCTGGAGCTGCCCAAGACTACTCAACCCGAATAAACTACATGAGCG
CGGGACCCCACATGATATCCCGGGTCAACGGAATACGCGCCCACCGAAACCGAATTCTCCTGGAA
CAGGCGGCTATTACCACCACACCTCGTAATAACCTTAATCCCCGTAGTTGGCCCGCTGCCCTGGTG
TACCAGGAAAGTCCCGCTCCCACCACTGTGGTACTTCCCAGAGACGCCCAGGCCGAAGTTCAGAT
GACTAACTCAGGGGCGCAGCTTGCGGGCGGCTTTCGTCACAGGGTGCGGTCGCCCGGGCAGGGTA
TAACTCACCTGACAATCAGAGGGCGAGGTATTCAGCTCAACGACGAGTCGGTGAGCTCCTCGCTTG
GTCTCCGTCCGGACGGGACATTTCAGATCGGCGGCGCCGGCCGCTCTTCATTCACGCCTCGTCAGG
CAATCCTAACTCTGCAGACCTCGTCCTCTGAGCCGCGCTCTGGAGGCATTGGAACTCTGCAATTTA
TTGAGGAGTTTGTGCCATCGGTCTACTTTAACCCCTTCTCGGGACCTCCCGGCCACTATCCGGATCA
ATTTATTCCTAACTTTGACGCGGTAAAGGACTCGGCGGACGGCTACGACTGAATGTTAAGTGGAGA
GGCAGAGCAACTGCGCCTGAAACACCTGGTCCACTGTCGCCGCCACAAGTGCTTTGCCCGCGACTC
CGGTGAGTTTTGCTACTTTGAATTGCCCGAGGATCATATCGAGGGCCCGGCGCACGGCGTCCGGCT
TACCGCCCAGGGAGAGCTTGCCCGTAGCCTGATTCGGGAGTTTACCCAGCGCCCCCTGCTAGTTGA
GCGGGACAGGGGACCCTGTGTTCTCACTGTGATTTGCAACTGTCCTAACCCTGGATTACATCAAGA
TCTTTGTTGCCATCTCTGTGCTGAGTATAATAAATACAGAAATTAAAATATACTGGGGCTCCTATC
GCCATCCTGTAAACGCCACCGTCTTCACCCGCCCAAGCAAACCAAGGCGAACCTTACCTGGTACTT
TTAACATCTCTCCCTCTGTGATTTACAACAGTTTCAACCCAGACGGAGTGAGTCTACGAGAGAACC
TCTCCGAGCTCAGCTACTCCATCAGAAAAAACACCACCCTCCTTACCTGCCGGGAACGTACGAGTG
CGTCACCGGCCGCTGCACCACACCTACCGCCTGACCGTAAACCAGACTTTTTCCGGACAGACCTCA
ATAACTCTGTTTACCAGAACAGGAGGTGAGCTTAGAAAACCCTTAGGGTATTAGGCCAAAGGCGC
AGCTACTGTGGGGTTTATGAACAATTCAAGCAACTCTACGGGCTATTCTAATTCAGGTTTCTCTAG
AAATGGACGGAATTATTACAGAGCAGCGCCTGCTAGAAAGACGCAGGGCAGCGGCCGAGCAACA
GCGCATGAATCAAGAGCTCCAAGACATGGTTAACTTGCACCAGTGCAAAAGGGGTATCTTTTGTCT
GGTAAAGCAGGCCAAAGTCACCTACGACAGTAATACCACCGGACACCGCCTTAGCTACAAGTTGC
CAACCAAGCGTCAGAAATTGGTGGTCATGGTGGGAGAAAAGCCCATTACCATAACTCAGCACTCG
GTAGAAACCGAAGGCTGCATTCACTCACCTTGTCAAGGACCTGAGGATCTCTGCACCCTTATTAAG
ACCCTGTGCGGTCTCAAAGATCTTATTCCCTTTAACTAATAAAAAAAAATAATAAAGCATCACTTA
CTTAAAATCAGTTAGCAAATTTCTGTCCAGTTTATTCAGCAGCACCTCCTTGCCCTCCTCCCAGCTC
TGGTATTGCAGCTTCCTCCTGGCTGCAAACTTTCTCCACAATCTAAATGGAATGTCAGTTTCCTCCT
GTTCCTGTCCATCCGCACCCACTATCTTCATGTTGTTGCAGATGAAGCGCGCAAGACCGTCTGAAG
ATACCTTCAACCCCGTGTATCCATATGACACGGAAACCGGTCCTCCAACTGTGCCTTTTCTTACTCC
TCCCTTTGTATCCCCCAATGGGTTTCAAGAGAGTCCCCCTGGGGTACTCTCTTTGCGCCTATCCGAA
CCTCTAGTTACCTCCAATGGCATGCTTGCGCTCAAAATGGGCAACGGCCTCTCTCTGGACGAGGCC
GGCAACCTTACCTCCCAAAATGTAACCACTGTGAGCCCACCTCTCAAAAAAACCAAGTCAAACAT
AAACCTGGAAATATCTGCACCCCTCACAGTTACCTCAGAAGCCCTAACTGTGGCTGCCGCCGCACC
TCTAATGGTCGCGGGCAACACACTCACCATGCAATCACAGGCCCCGCTAACCGTGCACGACTCCA
AACTTAGCATTGCCACCCAAGGACCCCTCACAGTGTCAGAAGGAAAGCTAGCCCTGCAAACATCA
GGCCCCCTCACCACCACCGATAGCAGTACCCTTACTATCACTGCCTCACCCCCTCTAACTACTGCC
ACTGGTAGCTTGGGCATTGACTTGAAAGAGCCCATTTATACACAAAATGGAAAACTAGGACTAAA
GTACGGGGCTCCTTTGCATGTAACAGACGACCTAAACACTTTGACCGTAGCAACTGGTCCAGGTGT
GACTATTAATAATACTTCCTTGCAAACTAAAGTTACTGGAGCCTTGGGTTTTGATTCACAAGGCAA
TATGCAACTTAATGTAGCAGGAGGACTAAGGATTGATTCTCAAAACAGACGCCTTATACTTGATGT
TAGTTATCCGTTTGATGCTCAAAACCAACTAAATCTAAGACTAGGACAGGGCCCTCTTTTTATAAA
CTCAGCCCACAACTTGGATATTAACTACAACAAAGGCCTTTACTTGTTTACAGCTTCAAACAATTC
CAAAAAGCTTGAGGTTAACCTAAGCACTGCCAAGGGGTTGATGTTTGACGCTACAGCCATAGCCA
TTAATGCAGGAGATGGGCTTGAATTTGGTTCACCTAATGCACCAAACACAAATCCCCTCAAAACAA
AAATTGGCCATGGCCTAGAATTTGATTCAAACAAGGCTATGGTTCCTAAACTAGGAACTGGCCTTA
GTTTTGACAGCACAGGTGCCATTACAGTAGGAAACAAAAATAATGATAAGCTAACTTTGTGGACC
ACACCAGCTCCATCTCCTAACTGTAGACTAAATGCAGAGAAAGATGCTAAACTCACTTTGGTCTTA
ACAAAATGTGGCAGTCAAATACTTGCTACAGTTTCAGTTTTGGCTGTTAAAGGCAGTTTGGCTCCA
ATATCTGGAACAGTTCAAAGTGCTCATCTTATTATAAGATTTGACGAAAATGGAGTGCTACTAAAC
AATTCCTTCCTGGACCCAGAATATTGGAACTTTAGAAATGGAGATCTTACTGAAGGCACAGCCTAT
ACAAACGCTGTTGGATTTATGCCTAACCTATCAGCTTATCCAAAATCTCACGGTAAAACTGCCAAA
AGTAACATTGTCAGTCAAGTTTACTTAAACGGAGACAAAACTAAACCTGTAACACTAACCATTACA
CTAAACGGTACACAGGAAACAGGAGACACAACTCCAAGTGCATACTCTATGTCATTTTCATGGGA
CTGGTCTGGCCACAACTACATTAATGAAATATTTGCCACATCCTCTTACACTTTTTCATACATTGCC
CAAGAATAAAGAATCGTTTGTGTTATGTTTCAACGTGTTTATTTTTCAATTGCAGAAAATTTCGAAT
CATTTTTCATTCAGTAGTATAGCCCCACCACCACATAGCTTATACAGATCACCGTACCTTAATCAA
ACTCACAGAACCCTAGTATTCAACCTGCCACCTCCCTCCCAACACACAGAGTACACAGTCCTTTCT
CCCCGGCTGGCCTTAAAAAGCATCATATCATGGGTAACAGACATATTCTTAGGTGTTATATTCCAC
ACGGTTTCCTGTCGAGCCAAACGCTCATCAGTGATATTAATAAACTCCCCGGGCAGCTCACTTAAG
TTCATGTCGCTGTCCAGCTGCTGAGCCACAGGCTGCTGTCCAACTTGCGGTTGCTTAACGGGCGGC
GAAGGAGAAGTCCACGCCTACATGGGGGTAGAGTCATAATCGTGCATCAGGATAGGGCGGTGGTG
CTGCAGCAGCGCGCGAATAAACTGCTGCCGCCGCCGCTCCGTCCTGCAGGAATACAACATGGCAG
TGGTCTCCTCAGCGATGATTCGCACCGCCCGCAGCATAAGGCGCCTTGTCCTCCGGGCACAGCAGC
GCACCCTGATCTCACTTAAATCAGCACAGTAACTGCAGCACAGCACCACAATATTGTTCAAAATCC
CACAGTGCAAGGCGCTGTATCCAAAGCTCATGGCGGGGACCACAGAACCCACGTGGCCATCATAC
CACAAGCGCAGGTAGATTAAGTGGCGACCCCTCATAAACACGCTGGACATAAACATTACCTCTTTT
GGCATGTTGTAATTCACCACCTCCCGGTACCATATAAACCTCTGATTAAACATGGCGCCATCCACC
ACCATCCTAAACCAGCTGGCCAAAACCTGCCCGCCGGCTATACACTGCAGGGAACCGGGACTGGA
ACAATGACAGTGGAGAGCCCAGGACTCGTAACCATGGATCATCATGCTCGTCATGATATCAATGTT
GGCACAACACAGGCACACGTGCATACACTTCCTCAGGATTACAAGCTCCTCCCGCGTTAGAACCAT
ATCCCAGGGAACAACCCATTCCTGAATCAGCGTAAATCCCACACTGCAGGGAAGACCTCGCACGT
AACTCACGTTGTGCATTGTCAAAGTGTTACATTCGGGCAGCAGCGGATGATCCTCCAGTATGGTAG
CGCGGGTTTCTGTCTCAAAAGGAGGTAGACGATCCCTACTGTACGGAGTGCGCCGAGACAACCGA
GATCGTGTTGGTCGTAGTGTCATGCCAAATGGAACGCCGGACGTAGTCATATTTCCTGAAGCAAAA
CCAGGTGCGGGCGTGACAAACAGATCTGCGTCTCCGGTCTCGCCGCTTAGATCGCTCTGTGTAGTA
GTTGTAGTATATCCACTCTCTCAAAGCATCCAGGCGCCCCCTGGCTTCGGGTTCTATGTAAACTCCT
TCATGCGCCGCTGCCCTGATAACATCCACCACCGCAGAATAAGCCACACCCAGCCAACCTACACAT
TCGTTCTGCGAGTCACACACGGGAGGAGCGGGAAGAGCTGGAAGAACCATGTTTTTTTTTTTATTC
CAAAAGATTATCCAAAACCTCAAAATGAAGATCTATTAAGTGAACGCGCTCCCCTCCGGTGGCGT
GGTCAAACTCTACAGCCAAAGAACAGATAATGGCATTTGTAAGATGTTGCACAATGGCTTCCAAA
AGGCAAACGGCCCTCACGTCCAAGTGGACGTAAAGGCTAAACCCTTCAGGGTGAATCTCCTCTAT
AAACATTCCAGCACCTTCAACCATGCCCAAATAATTCTCATCTCGCCACCTTCTCAATATATCTCTA
AGCAAATCCCGAATATTAAGTCCGGCCATTGTAAAAATCTGCTCCAGAGCGCCCTCCACCTTCAGC
CTCAAGCAGCGAATCATGATTGCAAAAATTCAGGTTCCTCACAGACCTGTATAAGATTCAAAAGC
GGAACATTAACAAAAATACCGCGATCCCGTAGGTCCCTTCGCAGGGCCAGCTGAACATAATCGTG
CAGGTCTGCACGGACCAGCGCGGCCACTTCCCCGCCAGGAACCATGACAAAAGAACCCACACTGA
TTATGACACGCATACTCGGAGCTATGCTAACCAGCGTAGCCCCGATGTAAGCTTGTTGCATGGGCG
GCGATATAAAATGCAAGGTGCTGCTCAAAAAATCAGGCAAAGCCTCGCGCAAAAAAGAAAGCAC
ATCGTAGTCATGCTCATGCAGATAAAGGCAGGTAAGCTCCGGAACCACCACAGAAAAAGACACCA
TTTTTCTCTCAAACATGTCTGCGGGTTTCTGCATAAACACAAAATAAAATAACAAAAAAACATTTA
AACATTAGAAGCCTGTCTTACAACAGGAAAAACAACCCTTATAAGCATAAGACGGACTACGGCCA
TGCCGGCGTGACCGTAAAAAAACTGGTCACCGTGATTAAAAAGCACCACCGACAGCTCCTCGGTC
ATGTCCGGAGTCATAATGTAAGACTCGGTAAACACATCAGGTTGATTCACATCGGTCAGTGCTAAA
AAGCGACCGAAATAGCCCGGGGGAATACATACCCGCAGGCGTAGAGACAACATTACAGCCCCCAT
AGGAGGTATAACAAAATTAATAGGAGAGAAAAACACATAAACACCTGAAAAACCCTCCTGCCTAG
GCAAAATAGCACCCTCCCGCTCCAGAACAACATACAGCGCTTCCACAGCGGCAGCCATAACAGTC
AGCCTTACCAGTAAAAAAGAAAACCTATTAAAAAAACACCACTCGACACGGCACCAGCTCAATCA
GTCACAGTGTAAAAAAGGGCCAAGTGCAGAGCGAGTATATATAGGACTAAAAAATGACGTAACG
GTTAAAGTCCACAAAAAACACCCAGAAAACCGCACGCGAACCTACGCCCAGAAACGAAAGCCAA
AAAACCCACAACTTCCTCAAATCGTCACTTCCGTTTTCCCACGTTACGTCACTTCCCATTTTAAGAA
AACTACAATTCCCAACACATACAAGTTACTCCGCCCTAAAACCTACGTCACCCGCCCCGTTCCCAC
GCCCCGCGCCACGTCACAAACTCCACCCCCTCATTATCATATTGGCTTCAATCCAAAATAAGGTAT
ATTATTGATGATGTTAATTAATTTAAATCCGCATGCGATATCGAGCTCTCCCGGGAATTCGGATCT
GCGACGCGAGGCTGGATGGCCTTCCCCATTATGATTCTTCTCGCGTTTAAGGGCACCAATAACTGC
CTTAAAAAAATTACGCCCCGCCCTGCCACTCATCGCAGTACTGTTGTAATTCATTAAGCATTCTGCC
GACATGGAAGCCATCACAAACGGCATGATGAACCTGAATCGCCAGCGGCATCAGCACCTTGTCGC
CTTGCGTATAATATTTGCCCATGGTGAAAACGGGGGCGAAGAAGTTGTCCATATTGGCCACGTTTA
AATCAAAACTGGTGAAACTCACCCAGGGATTGGCTGAGACGAAAAACATATTCTCAATAAACCCT
TTAGGGAAATAGGCCAGGTTTTCACCGTAACACGCCACATCTTGCGAATATATGTGTAGAAACTGC
CGGAAATCGTCGTGGTATTCACTCCAGAGCGATGAAAACGTTTCAGTTTGCTCATGGAAAACGGTG
TAACAAGGGTGAACACTATCCCATATCACCAGCTCACCGTCTTTCATTGCCATACGGAATTCCGGA
TGAGCATTCATCAGGCGGGCAAGAATGTGAATAAAGGCCGGATAAAACTTGTGCTTATTTTTCTTT
ACGGTCTTTAAAAAGGCCGTAATATCCAGCTGAACGGTCTGGTTATAGGTACATTGAGCAACTGAC
TGAAATGCCTCAAAATGTTCTTTACGATGCCATTGGGATATATCAACGGTGGTATATCCAGTGATT
TTTTTCTCCATTTTAGCTTCCTTAGCTCCTGAAAATCTCGATAACTCAAAAAATACGCCCGGTAGTG
ATCTTATTTCATTATGGTGAAAGTTGGAACCTCTTACGTGCCGATCAACGTCTCATTTTCGCCAAAA
GTTGGCCCAGGGCTTCCCGGTATCAACAGGGACACCAGGATTTATTTATTCTGCGAAGTGATCTTC
CGTCACAGGTATTTATTCGCGATAAGCTCATGGAGCGGCGTAACCGTCGCACAGGAAGGACAGAG
AAAGCGCGGATCTGGGAAGTGACGGACAGAACGGTCAGGACCTGGATTGGGGAGGCGGTTGCCG
CCGCTGCTGCTGACGGTGTGACGTTCTCTGTTCCGGTCACACCACATACGTTCCGCCATTCCTATGC
GATGCACATGCTGTATGCCGGTATACCGCTGAAAGTTCTGCAAAGCCTGATGGGACATAAGTCCAT
CAGTTCAACGGAAGTCTACACGAAGGTTTTTGCGCTGGATGTGGCTGCCCGGCACCGGGTGCAGTT
TGCGATGCCGGAGTCTGATGCGGTTGCGATGCTGAAACAATTATCCTGAGAATAAATGCCTTGGCC
TTTATATGGAAATGTGGAACTGAGTGGATATGCTGTTTTTGTCTGTTAAACAGAGAAGCTGGCTGT
TATCCACTGAGAAGCGAACGAAACAGTCGGGAAAATCTCCCATTATCGTAGAGATCCGCATTATT
AATCTCAGGAGCCTGTGTAGCGTTTATAGGAAGTAGTGTTCTGTCATGATGCCTGCAAGCGGTAAC
GAAAACGATTTGAATATGCCTTCAGGAACAATAGAAATCTTCGTGCGGTGTTACGTTGAAGTGGA
GCGGATTATGTCAGCAATGGACAGAACAACCTAATGAACACAGAACCATGATGTGGTCTGTCCTTT
TACAGCCAGTAGTGCTCGCCGCAGTCGAGCGACAGGGCGAAGCCCTCGAGTGAGCGAGGAAGCAC
CAGGGAACAGCACTTATATATTCTGCTTACACACGATGCCTGAAAAAACTTCCCTTGGGGTTATCC
ACTTATCCACGGGGATATTTTTATAATTATTTTTTTTATAGTTTTTAGATCTTCTTTTTTAGAGCGCC
TTGTAGGCCTTTATCCATGCTGGTTCTAGAGAAGGTGTTGTGACAAATTGCCCTTTCAGTGTGACA
AATCACCCTCAAATGACAGTCCTGTCTGTGACAAATTGCCCTTAACCCTGTGACAAATTGCCCTCA
GAAGAAGCTGTTTTTTCACAAAGTTATCCCTGCTTATTGACTCTTTTTTATTTAGTGTGACAATCTA
AAAACTTGTCACACTTCACATGGATCTGTCATGGCGGAAACAGCGGTTATCAATCACAAGAAACG
TAAAAATAGCCCGCGAATCGTCCAGTCAAACGACCTCACTGAGGCGGCATATAGTCTCTCCCGGG
ATCAAAAACGTATGCTGTATCTGTTCGTTGACCAGATCAGAAAATCTGATGGCACCCTACAGGAAC
ATGACGGTATCTGCGAGATCCATGTTGCTAAATATGCTGAAATATTCGGATTGACCTCTGCGGAAG
CCAGTAAGGATATACGGCAGGCATTGAAGAGTTTCGCGGGGAAGGAAGTGGTTTTTTATCGCCCT
GAAGAGGATGCCGGCGATGAAAAAGGCTATGAATCTTTTCCTTGGTTTATCAAACGTGCGCACAGT
CCATCCAGAGGGCTTTACAGTGTACATATCAACCCATATCTCATTCCCTTCTTTATCGGGTTACAGA
ACCGGTTTACGCAGTTTCGGCTTAGTGAAACAAAAGAAATCACCAATCCGTATGCCATGCGTTTAT
ACGAATCCCTGTGTCAGTATCGTAAGCCGGATGGCTCAGGCATCGTCTCTCTGAAAATCGACTGGA
TCATAGAGCGTTACCAGCTGCCTCAAAGTTACCAGCGTATGCCTGACTTCCGCCGCCGCTTCCTGC
AGGTCTGTGTTAATGAGATCAACAGCAGAACTCCAATGCGCCTCTCATACATTGAGAAAAAGAAA
GGCCGCCAGACGACTCATATCGTATTTTCCTTCCGCGATATCACTTCCATGACGACAGGATAGTCT
GAGGGTTATCTGTCACAGATTTGAGGGTGGTTCGTCACATTTGTTCTGACCTACTGAGGGTAATTT
GTCACAGTTTTGCTGTTTCCTTCAGCCTGCATGGATTTTCTCATACTTTTTGAACTGTAATTTTTAAG
GAAGCCAAATTTGAGGGCAGTTTGTCACAGTTGATTTCCTTCTCTTTCCCTTCGTCATGTGACCTGA
TATCGGGGGTTAGTTCGTCATCATTGATGAGGGTTGATTATCACAGTTTATTACTCTGAATTGGCTA
TCCGCGTGTGTACCTCTACCTGGAGTTTTTCCCACGGTGGATATTTCTTCTTGCGCTGAGCGTAAGA
GCTATCTGACAGAACAGTTCTTCTTTGCTTCCTCGCCAGTTCGCTCGCTATGCTCGGTTACACGGCT
GCGGCGAGCGCTAGTGATAATAAGTGACTGAGGTATGTGCTCTTCTTATCTCCTTTTGTAGTGTTGC
TCTTATTTTAAACAACTTTGCGGTTTTTTGATGACTTTGCGATTTTGTTGTTGCTTTGCAGTAAATTG
CAAGATTTAATAAAAAAACGCAAAGCAATGATTAAAGGATGTTCAGAATGAAACTCATGGAAACA
CTTAACCAGTGCATAAACGCTGGTCATGAAATGACGAAGGCTATCGCCATTGCACAGTTTAATGAT
GACAGCCCGGAAGCGAGGAAAATAACCCGGCGCTGGAGAATAGGTGAAGCAGCGGATTTAGTTG
GGGTTTCTTCTCAGGCTATCAGAGATGCCGAGAAAGCAGGGCGACTACCGCACCCGGATATGGAA
ATTCGAGGACGGGTTGAGCAACGTGTTGGTTATACAATTGAACAAATTAATCATATGCGTGATGTG
TTTGGTACGCGATTGCGACGTGCTGAAGACGTATTTCCACCGGTGATCGGGGTTGCTGCCCATAAA
GGTGGCGTTTACAAAACCTCAGTTTCTGTTCATCTTGCTCAGGATCTGGCTCTGAAGGGGCTACGT
GTTTTGCTCGTGGAAGGTAACGACCCCCAGGGAACAGCCTCAATGTATCACGGATGGGTACCAGA
TCTTCATATTCATGCAGAAGACACTCTCCTGCCTTTCTATCTTGGGGAAAAGGACGATGTCACTTAT
GCAATAAAGCCCACTTGCTGGCCGGGGCTTGACATTATTCCTTCCTGTCTGGCTCTGCACCGTATTG
AAACTGAGTTAATGGGCAAATTTGATGAAGGTAAACTGCCCACCGATCCACACCTGATGCTCCGA
CTGGCCATTGAAACTGTTGCTCATGACTATGATGTCATAGTTATTGACAGCGCGCCTAACCTGGGT
ATCGGCACGATTAATGTCGTATGTGCTGCTGATGTGCTGATTGTTCCCACGCCTGCTGAGTTGTTTG
ACTACACCTCCGCACTGCAGTTTTTCGATATGCTTCGTGATCTGCTCAAGAACGTTGATCTTAAAGG
GTTCGAGCCTGATGTACGTATTTTGCTTACCAAATACAGCAATAGTAATGGCTCTCAGTCCCCGTG
GATGGAGGAGCAAATTCGGGATGCCTGGGGAAGCATGGTTCTAAAAAATGTTGTACGTGAAACGG
ATGAAGTTGGTAAAGGTCAGATCCGGATGAGAACTGTTTTTGAACAGGCCATTGATCAACGCTCTT
CAACTGGTGCCTGGAGAAATGCTCTTTCTATTTGGGAACCTGTCTGCAATGAAATTTTCGATCGTCT
GATTAAACCACGCTGGGAGATTAGATAATGAAGCGTGCGCCTGTTATTCCAAAACATACGCTCAAT
ACTCAACCGGTTGAAGATACTTCGTTATCGACACCAGCTGCCCCGATGGTGGATTCGTTAATTGCG
CGCGTAGGAGTAATGGCTCGCGGTAATGCCATTACTTTGCCTGTATGTGGTCGGGATGTGAAGTTT
ACTCTTGAAGTGCTCCGGGGTGATAGTGTTGAGAAGACCTCTCGGGTATGGTCAGGTAATGAACGT
GACCAGGAGCTGCTTACTGAGGACGCACTGGATGATCTCATCCCTTCTTTTCTACTGACTGGTCAA
CAGACACCGGCGTTCGGTCGAAGAGTATCTGGTGTCATAGAAATTGCCGATGGGAGTCGCCGTCG
TAAAGCTGCTGCACTTACCGAAAGTGATTATCGTGTTCTGGTTGGCGAGCTGGATGATGAGCAGAT
GGCTGCATTATCCAGATTGGGTAACGATTATCGCCCAACAAGTGCTTATGAACGTGGTCAGCGTTA
TGCAAGCCGATTGCAGAATGAATTTGCTGGAAATATTTCTGCGCTGGCTGATGCGGAAAATATTTC
ACGTAAGATTATTACCCGCTGTATCAACACCGCCAAATTGCCTAAATCAGTTGTTGCTCTTTTTTCT
CACCCCGGTGAACTATCTGCCCGGTCAGGTGATGCACTTCAAAAAGCCTTTACAGATAAAGAGGA
ATTACTTAAGCAGCAGGCATCTAACCTTCATGAGCAGAAAAAAGCTGGGGTGATATTTGAAGCTG
AAGAAGTTATCACTCTTTTAACTTCTGTGCTTAAAACGTCATCTGCATCAAGAACTAGTTTAAGCTC
ACGACATCAGTTTGCTCCTGGAGCGACAGTATTGTATAAGGGCGATAAAATGGTGCTTAACCTGGA
CAGGTCTCGTGTTCCAACTGAGTGTATAGAGAAAATTGAGGCCATTCTTAAGGAACTTGAAAAGCC
AGCACCCTGATGCGACCACGTTTTAGTCTACGTTTATCTGTCTTTACTTAATGTCCTTTGTTACAGG
CCAGAAAGCATAACTGGCCTGAATATTCTCTCTGGGCCCACTGTTCCACTTGTATCGTCGGTCTGAT
AATCAGACTGGGACCACGGTCCCACTCGTATCGTCGGTCTGATTATTAGTCTGGGACCACGGTCCC
ACTCGTATCGTCGGTCTGATTATTAGTCTGGGACCACGGTCCCACTCGTATCGTCGGTCTGATAATC
AGACTGGGACCACGGTCCCACTCGTATCGTCGGTCTGATTATTAGTCTGGGACCATGGTCCCACTC
GTATCGTCGGTCTGATTATTAGTCTGGGACCACGGTCCCACTCGTATCGTCGGTCTGATTATTAGTC
TGGAACCACGGTCCCACTCGTATCGTCGGTCTGATTATTAGTCTGGGACCACGGTCCCACTCGTAT
CGTCGGTCTGATTATTAGTCTGGGACCACGATCCCACTCGTGTTGTCGGTCTGATTATCGGTCTGGG
ACCACGGTCCCACTTGTATTGTCGATCAGACTATCAGCGTGAGACTACGATTCCATCAATGCCTGT
CAAGGGCAAGTATTGACATGTCGTCGTAACCTGTAGAACGGAGTAACCTCGGTGTGCGGTTGTATG
CCTGCTGTGGATTGCTGCTGTGTCCTGCTTATCCACAACATTTTGCGCACGGTTATGTGGACAAAAT
ACCTGGTTACCCAGGCCGTGCCGGCACGTTAACCGGGCACATTTCCCCGAAAAGTGCCACCTGACG
TCTAAGAAACCATTATTATCATGACATTAACCTATAAAAATAGGCGTATCACGAGGCCCTTTCGTC
TTCAAGAATTGGATCCGAATTCCCGGGAGAGCTCGATATCGCATGCGGATTTAAATTAATTAA
* tadA-Only del araA-leu7697 insertion
(SEQ ID NO: 88)
ACGGCGTCCGCAACCGGACGATAATTTTTCTGCTCTTCAACGAACTGCGCAAAATCGTGGAAACGG
TTCGGGTCCAGCAGACGCAGACGGGCGAAGTGGCTTTCCATCCCCAGCTGTTCCGGGGTCGCGGTC
AGCAGCAGAACGCCCGGCACGTGCTCTGCCAGTTGTTCAATGGCCTGATTCGAGAAAGAGTGTTG
ACTTGTGAGCGGATAACAATGATACTTAGATTCAATTGTGAGCGGATAACAATTTCACACAGGCTA
GCGAATTCGAGCTCCCTCTAGAAATAATTTTGTTTAACTTTAAGAAGGAGATATACCATGGGCAGC
AGCTACCCATACGACGTACCAGATTACGCTTCCGAAGTCGAGTTTTCCCATGAGTACTGGATGAGA
CACGCATTGACTCTCGCAAAGAGGGCTCGAGATGAACGCGAGGTGCCCGTGGGGGCAGTACTCGT
GCTCAACAATCGCGTAATCGGCGAAGGTTGGAATAGGGCAATCGGACTCCACGACCCCACTGCAC
ATGCGGAAATCATGGCCCTTCGACAGGGAGGGCTTGTGATGCAGAATTATCGACTTATCGATGCG
ACGCTGTACGTCACGTTTGAACCTTGCGTAATGTGCGCGGGAGCTATGATTCACTCCCGCATTGGA
CGAGTTGTATTCGGTGTTCGCAACGCCAAGACGGGTGCCGCAGGTTCACTGATGGACGTGCTGCAT
TACCCAGGCATGAACCACCGGGTAGAAATCACAGAAGGCATATTGGCGGACGAATGTGCGGCGCT
GTTGTGTTACTTTTTTCGCATGCCCAGGCAGGTCTTTAACGCCCAGAAAAAAGCACAATCCTCTAC
TGACTTGAACGCCAGGCGCGGCAACGGGGTTATCAACTGCTGATTGCCTGCTCAGAAGATCAGCC
AGACAACGAAATGCGGTGCATTGAGCACCTTTTACAGCGTCAGGTTGATGCCATTATTGTTTCGAC
GTCGTTGCCTCCTGAGCATCCTTTTTATCAACGCTGGGCTAACGACCCGTTCCCGATTGTCGCGCTG
G
* tadA-XTEN-T7 del araA-leu7697 insertion
(SEQ ID NO: 89)
ACGGCGTCCGCAACCGGACGATAATTTTTCTGCTCTTCAACGAACTGCGCAAAATCGTGGAAACGG
TTCGGGTCCAGCAGACGCAGACGGGCGAAGTGGCTTTCCATCCCCAGCTGTTCCGGGGTCGCGGTC
AGCAGCAGAACGCCCGGCACGTGCTCTGCCAGTTGTTCAATGGCCTGATTCGAGAAAGAGTGTTG
ACTTGTGAGCGGATAACAATGATACTTAGATTCAATTGTGAGCGGATAACAATTTCACACAGGCTA
GCGAATTCGAGCTCCCTCTAGAAATAATTTTGTTTAACTTTAAGAAGGAGATATACCATGGGCAGC
AGCTACCCATACGACGTACCAGATTACGCTTCCGAAGTCGAGTTTTCCCATGAGTACTGGATGAGA
CACGCATTGACTCTCGCAAAGAGGGCTCGAGATGAACGCGAGGTGCCCGTGGGGGCAGTACTCGT
GCTCAACAATCGCGTAATCGGCGAAGGTTGGAATAGGGCAATCGGACTCCACGACCCCACTGCAC
ATGCGGAAATCATGGCCCTTCGACAGGGAGGGCTTGTGATGCAGAATTATCGACTTATCGATGCG
ACGCTGTACGTCACGTTTGAACCTTGCGTAATGTGCGCGGGAGCTATGATTCACTCCCGCATTGGA
CGAGTTGTATTCGGTGTTCGCAACGCCAAGACGGGTGCCGCAGGTTCACTGATGGACGTGCTGCAT
TACCCAGGCATGAACCACCGGGTAGAAATCACAGAAGGCATATTGGCGGACGAATGTGCGGCGCT
GTTGTGTTACTTTTTTCGCATGCCCAGGCAGGTCTTTAACGCCCAGAAAAAAGCACAATCCTCTAC
TGACTCTGGTGGTTCTTCTGGTGGTTCTAGCGGCAGCGAGACTCCCGGGACCTCAGAGTCCGCCAC
ACCCGAAAGTTCTGGTGGTTCTTCTGGTGGTTCTAGAGGATACCATATGAACACGATTAACATCGC
TAAGAACGACTTCTCTGACATCGAACTGGCTGCTATCCCGTTCAACACTCTGGCTGACCATTACGG
TGAGCGTTTAGCTCGCGAACAGTTGGCCCTTGAGCATGAGTCTTACGAGATGGGTGAAGCACGCTT
CCGCAAGATGTTTGAGCGTCAACTTAAAGCTGGTGAGGTTGCGGATAACGCTGCCGCCAAGCCTCT
CATCACTACCCTACTCCCTAAGATGATTGCACGCATCAACGACTGGTTTGAGGAAGTGAAAGCTAA
GCGCGGCAAGCGCCCGACAGCCTTCCAGTTCCTGCAAGAAATCAAGCCGGAAGCCGTAGCGTACA
TCACCATTAAGACCACTCTGGCTTGCCTAACCAGTGCTGACAATACAACCGTTCAGGCTGTAGCAA
GCGCAATCGGTCGGGCCATTGAGGACGAGGCTCGCTTCGGTCGTATCCGTGACCTTGAAGCTAAGC
ACTTCAAGAAAAACGTTGAGGAACAACTCAACAAGCGCGTAGGGCACGTCTACAAGAAAGCATTT
ATGCAAGTTGTCGAGGCTGACATGCTCTCTAAGGGTCTACTCGGTGGCGAGGCGTGGTCTTCGTGG
CATAAGGAAGACTCTATTCATGTAGGAGTACGCTGCATCGAGATGCTCATTGAGTCAACCGGAAT
GGTTAGCTTACACCGCCAAAATGCTGGCGTAGTAGGTCAAGACTCTGAGACTATCGAACTCGCACC
TGAATACGCTGAGGCTATCGCAACCCGTGCAGGTGCGCTGGCTGGCATCTCTCCGATGTTCCAACC
TTGCGTAGTTCCTCCTAAGCCGTGGACTGGCATTACTGGTGGTGGCTATTGGGCTAACGGTCGTCG
TCCTCTGGCGCTGGTGCGTACTCACAGTAAGAAAGCACTGATGCGCTACGAAGACGTTTACATGCC
TGAGGTGTACAAAGCGATTAACATTGCGCAAAACACCGCATGGAAAATCAACAAGAAAGTCCTAG
CGGTCGCCAACGTAATCACCAAGTGGAAGCATTGTCCGGTCGAGGACATCCCTGCGATTGAGCGT
GAAGAACTCCCGATGAAACCGGAAGACATCGACATGAATCCTGAGGCTCTCACCGCGTGGAAACG
TGCTGCCGCTGCTGTGTACCGCAAGGACAAGGCTCGCAAGTCTCGCCGTATCAGCCTTGAGTTCAT
GCTTGAGCAAGCCAATAAGTTTGCTAACCATAAGGCCATCTGGTTCCCTTACAACATGGACTGGCG
CGGTCGTGTTTACGCTGTGTCAATGTTCAACCCGCAAGGTAACGATATGACCAAAGGACTGCTTAC
GCTGGCGAAAGGTAAACCAATCGGTAAGGAAGGTTACTACTGGCTGAAAATCCACGGTGCAAACT
GTGCGGGTGTCGATAAGGTTCCGTTCCCTGAGCGCATCAAGTTCATTGAGGAAAACCACGAGAAC
ATCATGGCTTGCGCTAAGTCTCCACTGGAGAACACTTGGTGGGCTGAGCAAGATTCTCCGTTCTGC
TTCCTTGCGTTCTGCTTTGAGTACGCTGGGGTACAGCACCACGGCCTGAGCTATAACTGCTCCCTTC
CGCTGGCGTTTGACGGGTCTTGCTCTGGCATCCAGCACTTCTCCGCGATGCTCCGAGATGAGGTAG
GTGGTCGCGCGGTTAACTTGCTTCCTAGTGAAACCGTTCAGGACATCTACGGGATTGTTGCTAAGA
AAGTCAACGAGATTCTACAAGCAGACGCAATCAATGGGACCGATAACGAAGTAGTTACCGTGACC
GATGAGAACACTGGTGAAATCTCTGAGAAAGTCAAGCTGGGCACTAAGGCACTGGCTGGTCAATG
GCTGGCTTACGGTGTTACTCGCAGTGTGACTAAGCGTTCAGTCATGACGCTGGCTTACGGGTCCAA
AGAGTTCGGCTTCCGTCAACAAGTGCTGGAAGATACCATTCAGCCAGCTATTGATTCCGGCAAGGG
TCTGATGTTCACTCAGCCGAATCAGGCTGCTGGATACATGGCTAAGCTGATTTGGGAATCTGTGAG
CGTGACGGTGGTAGCTGCGGTTGAAGCAATGAACTGGCTTAAGTCTGCTGCTAAGCTGCTGGCTGC
TGAGGTCAAAGATAAGAAGACTGGAGAGATTCTTCGCAAGCGTTGCGCTGTGCATTGGGTAACTC
CTGATGGTTTCCCTGTGTGGCAGGAATACAAGAAGCCTATTCAGACGCGCTTGAACCTGATGTTCC
TCGGTCAGTTCCGCTTACAGCCTACCATTAACACCAACAAAGATAGCGAGATTGATGCACACAAA
CAGGAGTCTGGTATCGCTCCTAACTTTGTACACAGCCAAGACGGTAGCCACCTTCGTAAGACTGTA
GTGTGGGCACACGAGAAGTACGGAATCGAATCTTTTGCACTGATTCACGACTCCTTCGGTACCATT
CCGGCTGACGCTGCGAACCTGTTCAAAGCAGTGCGCGAAACTATGGTTGACACATATGAGTCTTGT
GATGTACTGGCTGATTTCTACGACCAGTTCGCTGACCAGTTGCACGAGTCTCAATTGGACAAAATG
CCAGCACTTCCGGCTAAAGGTAACTTGAACCTCCGTGACATCTTAGAGTCGGACTTCGCGTTCGCG
TAATCTAGAGTCGACCTGCAGGCATGCAAGCTTGGCTGTTTTGGCGGATGAGAGAAGATTTTCAGT
TGAACGCCAGGCGCGGCAACGGGGTTATCAACTGCTGATTGCCTGCTCAGAAGATCAGCCAGACA
ACGAAATGCGGTGCATTGAGCACCTTTTACAGCGTCAGGTTGATGCCATTATTGTTTCGACGTCGT
TGCCTCCTGAGCATCCTTTTTATCAACGCTGGGCTAACGACCCGTTCCCGATTGTCGCGCTGG
* tadA-GGS-T7 del araA-leu7697 insertion
(SEQ ID NO: 90)
ACGGCGTCCGCAACCGGACGATAATTTTTCTGCTCTTCAACGAACTGCGCAAAATCGTGGAAACGG
TTCGGGTCCAGCAGACGCAGACGGGCGAAGTGGCTTTCCATCCCCAGCTGTTCCGGGGTCGCGGTC
AGCAGCAGAACGCCCGGCACGTGCTCTGCCAGTTGTTCAATGGCCTGATTCGAGAAAGAGTGTTG
ACTTGTGAGCGGATAACAATGATACTTAGATTCAATTGTGAGCGGATAACAATTTCACACAGGCTA
GCGAATTCGAGCTCCCTCTAGAAATAATTTTGTTTAACTTTAAGAAGGAGATATACCATGGGCAGC
AGCTACCCATACGACGTACCAGATTACGCTTCCGAAGTCGAGTTTTCCCATGAGTACTGGATGAGA
CACGCATTGACTCTCGCAAAGAGGGCTCGAGATGAACGCGAGGTGCCCGTGGGGGCAGTACTCGT
GCTCAACAATCGCGTAATCGGCGAAGGTTGGAATAGGGCAATCGGACTCCACGACCCCACTGCAC
ATGCGGAAATCATGGCCCTTCGACAGGGAGGGCTTGTGATGCAGAATTATCGACTTATCGATGCG
ACGCTGTACGTCACGTTTGAACCTTGCGTAATGTGCGCGGGAGCTATGATTCACTCCCGCATTGGA
CGAGTTGTATTCGGTGTTCGCAACGCCAAGACGGGTGCCGCAGGTTCACTGATGGACGTGCTGCAT
TACCCAGGCATGAACCACCGGGTAGAAATCACAGAAGGCATATTGGCGGACGAATGTGCGGCGCT
GTTGTGTTACTTTTTTCGCATGCCCAGGCAGGTCTTTAACGCCCAGAAAAAAGCACAATCCTCTAC
TGACGGCGGTAGCGGAGGGAGTGGCGGTAGCGGAGGGAGTGGGAGCTCAAGAGGATACCATATG
AACACGATTAACATCGCTAAGAACGACTTCTCTGACATCGAACTGGCTGCTATCCCGTTCAACACT
CTGGCTGACCATTACGGTGAGCGTTTAGCTCGCGAACAGTTGGCCCTTGAGCATGAGTCTTACGAG
ATGGGTGAAGCACGCTTCCGCAAGATGTTTGAGCGTCAACTTAAAGCTGGTGAGGTTGCGGATAA
CGCTGCCGCCAAGCCTCTCATCACTACCCTACTCCCTAAGATGATTGCACGCATCAACGACTGGTT
TGAGGAAGTGAAAGCTAAGCGCGGCAAGCGCCCGACAGCCTTCCAGTTCCTGCAAGAAATCAAGC
CGGAAGCCGTAGCGTACATCACCATTAAGACCACTCTGGCTTGCCTAACCAGTGCTGACAATACAA
CCGTTCAGGCTGTAGCAAGCGCAATCGGTCGGGCCATTGAGGACGAGGCTCGCTTCGGTCGTATCC
GTGACCTTGAAGCTAAGCACTTCAAGAAAAACGTTGAGGAACAACTCAACAAGCGCGTAGGGCAC
GTCTACAAGAAAGCATTTATGCAAGTTGTCGAGGCTGACATGCTCTCTAAGGGTCTACTCGGTGGC
GAGGCGTGGTCTTCGTGGCATAAGGAAGACTCTATTCATGTAGGAGTACGCTGCATCGAGATGCTC
ATTGAGTCAACCGGAATGGTTAGCTTACACCGCCAAAATGCTGGCGTAGTAGGTCAAGACTCTGA
GACTATCGAACTCGCACCTGAATACGCTGAGGCTATCGCAACCCGTGCAGGTGCGCTGGCTGGCAT
CTCTCCGATGTTCCAACCTTGCGTAGTTCCTCCTAAGCCGTGGACTGGCATTACTGGTGGTGGCTAT
TGGGCTAACGGTCGTCGTCCTCTGGCGCTGGTGCGTACTCACAGTAAGAAAGCACTGATGCGCTAC
GAAGACGTTTACATGCCTGAGGTGTACAAAGCGATTAACATTGCGCAAAACACCGCATGGAAAAT
CAACAAGAAAGTCCTAGCGGTCGCCAACGTAATCACCAAGTGGAAGCATTGTCCGGTCGAGGACA
TCCCTGCGATTGAGCGTGAAGAACTCCCGATGAAACCGGAAGACATCGACATGAATCCTGAGGCT
CTCACCGCGTGGAAACGTGCTGCCGCTGCTGTGTACCGCAAGGACAAGGCTCGCAAGTCTCGCCGT
ATCAGCCTTGAGTTCATGCTTGAGCAAGCCAATAAGTTTGCTAACCATAAGGCCATCTGGTTCCCT
TACAACATGGACTGGCGCGGTCGTGTTTACGCTGTGTCAATGTTCAACCCGCAAGGTAACGATATG
ACCAAAGGACTGCTTACGCTGGCGAAAGGTAAACCAATCGGTAAGGAAGGTTACTACTGGCTGAA
AATCCACGGTGCAAACTGTGCGGGTGTCGATAAGGTTCCGTTCCCTGAGCGCATCAAGTTCATTGA
GGAAAACCACGAGAACATCATGGCTTGCGCTAAGTCTCCACTGGAGAACACTTGGTGGGCTGAGC
AAGATTCTCCGTTCTGCTTCCTTGCGTTCTGCTTTGAGTACGCTGGGGTACAGCACCACGGCCTGAG
CTATAACTGCTCCCTTCCGCTGGCGTTTGACGGGTCTTGCTCTGGCATCCAGCACTTCTCCGCGATG
CTCCGAGATGAGGTAGGTGGTCGCGCGGTTAACTTGCTTCCTAGTGAAACCGTTCAGGACATCTAC
GGGATTGTTGCTAAGAAAGTCAACGAGATTCTACAAGCAGACGCAATCAATGGGACCGATAACGA
AGTAGTTACCGTGACCGATGAGAACACTGGTGAAATCTCTGAGAAAGTCAAGCTGGGCACTAAGG
CACTGGCTGGTCAATGGCTGGCTTACGGTGTTACTCGCAGTGTGACTAAGCGTTCAGTCATGACGC
TGGCTTACGGGTCCAAAGAGTTCGGCTTCCGTCAACAAGTGCTGGAAGATACCATTCAGCCAGCTA
TTGATTCCGGCAAGGGTCTGATGTTCACTCAGCCGAATCAGGCTGCTGGATACATGGCTAAGCTGA
TTTGGGAATCTGTGAGCGTGACGGTGGTAGCTGCGGTTGAAGCAATGAACTGGCTTAAGTCTGCTG
CTAAGCTGCTGGCTGCTGAGGTCAAAGATAAGAAGACTGGAGAGATTCTTCGCAAGCGTTGCGCT
GTGCATTGGGTAACTCCTGATGGTTTCCCTGTGTGGCAGGAATACAAGAAGCCTATTCAGACGCGC
TTGAACCTGATGTTCCTCGGTCAGTTCCGCTTACAGCCTACCATTAACACCAACAAAGATAGCGAG
ATTGATGCACACAAACAGGAGTCTGGTATCGCTCCTAACTTTGTACACAGCCAAGACGGTAGCCAC
CTTCGTAAGACTGTAGTGTGGGCACACGAGAAGTACGGAATCGAATCTTTTGCACTGATTCACGAC
TCCTTCGGTACCATTCCGGCTGACGCTGCGAACCTGTTCAAAGCAGTGCGCGAAACTATGGTTGAC
ACATATGAGTCTTGTGATGTACTGGCTGATTTCTACGACCAGTTCGCTGACCAGTTGCACGAGTCT
CAATTGGACAAAATGCCAGCACTTCCGGCTAAAGGTAACTTGAACCTCCGTGACATCTTAGAGTCG
GACTTCGCGTTCGCGTAATCTAGAGTCGACCTGCAGGCATGCAAGCTTGGCTGTTTTGGCGGATGA
GAGAAGATTTTCAGTTGAACGCCAGGCGCGGCAACGGGGTTATCAACTGCTGATTGCCTGCTCAG
AAGATCAGCCAGACAACGAAATGCGGTGCATTGAGCACCTTTTACAGCGTCAGGTTGATGCCATT
ATTGTTTCGACGTCGTTGCCTCCTGAGCATCCTTTTTATCAACGCTGGGCTAACGACCCGTTCCCGA
TTGTCGCGCTGG
* BAC-KanStop-TetStop
(SEQ ID NO: 91)
ACAATTAATCATCGGCTCGAAGCTTGTTGACAATTAATCATCGGCATAGTATATCGGCATAGTATA
ATACGACAAGGTGAGGAACTAAACCATGGGATCGGCCATTGAACAAGATGGATTGCACGCAGGTT
CTCCGGCCGCTTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCGACCTGTCCG
GTGCCCTGAATGAACTGCAGGACGAGGCAGCGCGGCTATCGTAGCTGGCCACGACGGGCGTTCCT
TGCGCAGCTGTGCTCGACGTTGTCACTGAAGCGGGAAGGGACTGGCTGCTATTGGGCGAAGTGCC
GGGGCAGGATCTCCTGTCATCTCACCTTGCTCCTGCCGAGAAAGTATCCATCATGGCTGATGCAAT
GCGGCGGCTGCATACGCTTGATCCGGCTACCTGCCCATTCGACCACCAAGCGAAACATCGCATCGA
GCGAGCACGTACTCGGATGGAAGCCGGTCTTGTCGATCAGGATGATCTGGACGAAGAGCATCAGG
GGCTCGCGCCAGCCGAACTGTTCGCCAGGCTCAAGGCGCGCATGCCCGACGGCGATGATCTCGTC
GTGACCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGATATTGCT
GAAGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGGGGATCAATTCTCTAGAGCTTAAT
TAACGCAGCCTGAATGGCGAATAGGGATCCTTGACAGCTTATCATCGATAAGCTTTAATGCGGTAG
TTTATCACAGTTGCTAACGCAGTCAGGCACCGTGTATGAATAGTTCGACAAAGATCGCATTGGTAA
TTACGTTACTCGATGCCATGGGGATTGGCCTTATCATGCCAGTCTTGCCAACGTTATTACGTGAATT
TATTGCTTCGGAAGATATCGCTAACCACTTTGGCGTATTGCTTGCACTTTATGCGTTAATGCAGGTT
ATCTTTGCTCCTTAGCTTGGAAAAATGTCTGACCGATTTGGTCGGCGCCCAGTGCTGTTGTTGTCAT
TAATAGGCGCATCGCTGGATTACTTATTGCTGGCTTTTTCAAGTGCGCTTTGGATGCTGTATTTAGG
CCGTTTGCTTTCAGGGATCACAGGAGCTACTGGGGCTGTCGCGGCATCGGTCATTGCCGATACCAC
CTCAGCTTCTCAACGCGTGAAGTGGTTCGGTTGGTTAGGGGCAAGTTTTGGGCTTGGTTTAATAGC
GGGGCCTATTATTGGTGGTTTTGCAGGAGAGATTTCACCGCATAGTCCCTTTTTTATCGCTGCGTTG
CTAAATATTGTCACTTTCCTTGTGGTTATGTTTTGGTTCCGTGAAACCAAAAATACACGTGATAATA
CAGATACCGAAGTAGGGGTTGAGACGCAATCGAATTCGGTATACATCACTTTATTTAAAACGATGC
CCATTTTGTTGATTATTTATTTTTCAGCGCAATTGATAGGCCAAATTCCCGCAACGGTGTGGGTGCT
ATTTACCGAAAATCGTTTTGGATGGAATAGCATGATGGTTGGCTTTTCATTAGCGGGTCTTGGTCTT
TTACACTCAGTATTCCAAGCCTTTGTGGCAGGAAGAATAGCCACTAAATGGGGCGAAAAAACGGC
AGTACTGCTCGGATTTATTGCAGATAGTAGTGCATTTGCCTTTTTAGCGTTTATATCTGAAGGTTGG
TTAGTTTTCCCTGTTTTAATTTTATTGGCTGGTGGTGGGATCGCTTTACCTGCATTACAGGGAGTGA
TGTCTATCCAAACAAAGAGTCATCAGCAAGGTGCTTTACAGGGATTATTGGTGAGCCTTACCAATG
CAACCGGTGTTATTGGCCCATTACTGTTTGCTGTTATTTATAATCATTCACTACCAATTTGGGATGG
CTGGATTTGGATTATTGGTTTAGCGTTTTACTGTATTATTATCCTGCTATCGATGACCTTCATGTTAA
CCCCTCAAGCTCAGGGGAGTAAACAGGAGACAAGTGCTTAGTGATCCAATTCTTGAAGACGAAAG
GGCCTCGTGATACGCCTATTTTTATAGGTTAATGTCATGATAATAATGGTTTCTTAGACGTCAGGTG
GCACTTTTCGGGGAAATGTGCCCGGTTAACGTGCCGGCACGGCCTGGGTAACCAGGTATTTTGTCC
ACATAACCGTGCGCAAAATGTTGTGGATAAGCAGGACACAGCAGCAATCCACAGCAGGCATACAA
CCGCACACCGAGGTTACTCCGTTCTACAGGTTACGACGACATGTCAATACTTGCCCTTGACAGGCA
TTGATGGAATCGTAGTCTCACGCTGATAGTCTGATCGACAATACAAGTGGGACCGTGGTCCCAGAC
CGATAATCAGACCGACAACACGAGTGGGATCGTGGTCCCAGACTAATAATCAGACCGACGATACG
AGTGGGACCGTGGTCCCAGACTAATAATCAGACCGACGATACGAGTGGGACCGTGGTTCCAGACT
AATAATCAGACCGACGATACGAGTGGGACCGTGGTCCCAGACTAATAATCAGACCGACGATACGA
GTGGGACCATGGTCCCAGACTAATAATCAGACCGACGATACGAGTGGGACCGTGGTCCCAGTCTG
ATTATCAGACCGACGATACGAGTGGGACCGTGGTCCCAGACTAATAATCAGACCGACGATACGAG
TGGGACCGTGGTCCCAGACTAATAATCAGACCGACGATACGAGTGGGACCGTGGTCCCAGTCTGA
TTATCAGACCGACGATACAAGTGGAACAGTGGGCCCAGAGAGAATATTCAGGCCAGTTATGCTTT
CTGGCCTGTAACAAAGGACATTAAGTAAAGACAGATAAACGTAGACTAAAACGTGGTCGCATCAG
GGTGCTGGCTTTTCAAGTTCCTTAAGAATGGCCTCAATTTTCTCTATACACTCAGTTGGAACACGAG
ACCTGTCCAGGTTAAGCACCATTTTATCGCCCTTATACAATACTGTCGCTCCAGGAGCAAACTGAT
GTCGTGAGCTTAAACTAGTTCTTGATGCAGATGACGTTTTAAGCACAGAAGTTAAAAGAGTGATAA
CTTCTTCAGCTTCAAATATCACCCCAGCTTTTTTCTGCTCATGAAGGTTAGATGCCTGCTGCTTAAG
TAATTCCTCTTTATCTGTAAAGGCTTTTTGAAGTGCATCACCTGACCGGGCAGATAGTTCACCGGG
GTGAGAAAAAAGAGCAACAACTGATTTAGGCAATTTGGCGGTGTTGATACAGCGGGTAATAATCT
TACGTGAAATATTTTCCGCATCAGCCAGCGCAGAAATATTTCCAGCAAATTCATTCTGCAATCGGC
TTGCATAACGCTGACCACGTTCATAAGCACTTGTTGGGCGATAATCGTTACCCAATCTGGATAATG
CAGCCATCTGCTCATCATCCAGCTCGCCAACCAGAACACGATAATCACTTTCGGTAAGTGCAGCAG
CTTTACGACGGCGACTCCCATCGGCAATTTCTATGACACCAGATACTCTTCGACCGAACGCCGGTG
TCTGTTGACCAGTCAGTAGAAAAGAAGGGATGAGATCATCCAGTGCGTCCTCAGTAAGCAGCTCC
TGGTCACGTTCATTACCTGACCATACCCGAGAGGTCTTCTCAACACTATCACCCCGGAGCACTTCA
AGAGTAAACTTCACATCCCGACCACATACAGGCAAAGTAATGGCATTACCGCGAGCCATTACTCCT
ACGCGCGCAATTAACGAATCCACCATCGGGGCAGCTGGTGTCGATAACGAAGTATCTTCAACCGG
TTGAGTATTGAGCGTATGTTTTGGAATAACAGGCGCACGCTTCATTATCTAATCTCCCAGCGTGGTT
TAATCAGACGATCGAAAATTTCATTGCAGACAGGTTCCCAAATAGAAAGAGCATTTCTCCAGGCA
CCAGTTGAAGAGCGTTGATCAATGGCCTGTTCAAAAACAGTTCTCATCCGGATCTGACCTTTACCA
ACTTCATCCGTTTCACGTACAACATTTTTTAGAACCATGCTTCCCCAGGCATCCCGAATTTGCTCCT
CCATCCACGGGGACTGAGAGCCATTACTATTGCTGTATTTGGTAAGCAAAATACGTACATCAGGCT
CGAACCCTTTAAGATCAACGTTCTTGAGCAGATCACGAAGCATATCGAAAAACTGCAGTGCGGAG
GTGTAGTCAAACAACTCAGCAGGCGTGGGAACAATCAGCACATCAGCAGCACATACGACATTAAT
CGTGCCGATACCCAGGTTAGGCGCGCTGTCAATAACTATGACATCATAGTCATGAGCAACAGTTTC
AATGGCCAGTCGGAGCATCAGGTGTGGATCGGTGGGCAGTTTACCTTCATCAAATTTGCCCATTAA
CTCAGTTTCAATACGGTGCAGAGCCAGACAGGAAGGAATAATGTCAAGCCCCGGCCAGCAAGTGG
GCTTTATTGCATAAGTGACATCGTCCTTTTCCCCAAGATAGAAAGGCAGGAGAGTGTCTTCTGCAT
GAATATGAAGATCTGGTACCCATCCGTGATACATTGAGGCTGTTCCCTGGGGGTCGTTACCTTCCA
CGAGCAAAACACGTAGCCCCTTCAGAGCCAGATCCTGAGCAAGATGAACAGAAACTGAGGTTTTG
TAAACGCCACCTTTATGGGCAGCAACCCCGATCACCGGTGGAAATACGTCTTCAGCACGTCGCAAT
CGCGTACCAAACACATCACGCATATGATTAATTTGTTCAATTGTATAACCAACACGTTGCTCAACC
CGTCCTCGAATTTCCATATCCGGGTGCGGTAGTCGCCCTGCTTTCTCGGCATCTCTGATAGCCTGAG
AAGAAACCCCAACTAAATCCGCTGCTTCACCTATTCTCCAGCGCCGGGTTATTTTCCTCGCTTCCGG
GCTGTCATCATTAAACTGTGCAATGGCGATAGCCTTCGTCATTTCATGACCAGCGTTTATGCACTG
GTTAAGTGTTTCCATGAGTTTCATTCTGAACATCCTTTAATCATTGCTTTGCGTTTTTTTATTAAATC
TTGCAATTTACTGCAAAGCAACAACAAAATCGCAAAGTCATCAAAAAACCGCAAAGTTGTTTAAA
ATAAGAGCAACACTACAAAAGGAGATAAGAAGAGCACATACCTCAGTCACTTATTATCACTAGCG
CTCGCCGCAGCCGTGTAACCGAGCATAGCGAGCGAACTGGCGAGGAAGCAAAGAAGAACTGTTCT
GTCAGATAGCTCTTACGCTCAGCGCAAGAAGAAATATCCACCGTGGGAAAAACTCCAGGTAGAGG
TACACACGCGGATAGCCAATTCAGAGTAATAAACTGTGATAATCAACCCTCATCAATGATGACGA
ACTAACCCCCGATATCAGGTCACATGACGAAGGGAAAGAGAAGGAAATCAACTGTGACAAACTGC
CCTCAAATTTGGCTTCCTTAAAAATTACAGTTCAAAAAGTATGAGAAAATCCATGCAGGCTGAAGG
AAACAGCAAAACTGTGACAAATTACCCTCAGTAGGTCAGAACAAATGTGACGAACCACCCTCAAA
TCTGTGACAGATAACCCTCAGACTATCCTGTCGTCATGGAAGTGATATCGCGGAAGGAAAATACG
ATATGAGTCGTCTGGCGGCCTTTCTTTTTCTCAATGTATGAGAGGCGCATTGGAGTTCTGCTGTTGA
TCTCATTAACACAGACCTGCAGGAAGCGGCGGCGGAAGTCAGGCATACGCTGGTAACTTTGAGGC
AGCTGGTAACGCTCTATGATCCAGTCGATTTTCAGAGAGACGATGCCTGAGCCATCCGGCTTACGA
TACTGACACAGGGATTCGTATAAACGCATGGCATACGGATTGGTGATTTCTTTTGTTTCACTAAGC
CGAAACTGCGTAAACCGGTTCTGTAACCCGATAAAGAAGGGAATGAGATATGGGTTGATATGTAC
ACTGTAAAGCCCTCTGGATGGACTGTGCGCACGTTTGATAAACCAAGGAAAAGATTCATAGCCTTT
TTCATCGCCGGCATCCTCTTCAGGGCGATAAAAAACCACTTCCTTCCCCGCGAAACTCTTCAATGC
CTGCCGTATATCCTTACTGGCTTCCGCAGAGGTCAATCCGAATATTTCAGCATATTTAGCAACATG
GATCTCGCAGATACCGTCATGTTCCTGTAGGGTGCCATCAGATTTTCTGATCTGGTCAACGAACAG
ATACAGCATACGTTTTTGATCCCGGGAGAGACTATATGCCGCCTCAGTGAGGTCGTTTGACTGGAC
GATTCGCGGGCTATTTTTACGTTTCTTGTGATTGATAACCGCTGTTTCCGCCATGACAGATCCATGT
GAAGTGTGACAAGTTTTTAGATTGTCACACTAAATAAAAAAGAGTCAATAAGCAGGGATAACTTT
GTGAAAAAACAGCTTCTTCTGAGGGCAATTTGTCACAGGGTTAAGGGCAATTTGTCACAGACAGG
ACTGTCATTTGAGGGTGATTTGTCACACTGAAAGGGCAATTTGTCACAACACCTTCTCTAGAACCA
GCATGGATAAAGGCCTACAAGGCGCTCTAAAAAAGAAGATCTAAAAACTATAAAAAAAATAATTA
TAAAAATATCCCCGTGGATAAGTGGATAACCCCAAGGGAAGTTTTTTCAGGCATCGTGTGTAAGCA
GAATATATAAGTGCTGTTCCCTGGTGCTTCCTCGCTCACTCGAGCTACGGGGTCTGACGCTCAGTG
GAACGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCATAGTTGCCTGA
CTGCCCGTCGTGTAGATAACTACGATACGGGAGGGCTTACCATCTGGCCCCAGTGCTGCAATGATA
CCGCGGGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGTTACATGATC
CCCCATGTTGTGAAAAAAAGCGGTTAGCTCCTTCGGTCCTCCGATCGTTGTCAGAAGTAAGTTGGC
CGCAGTGTTATCACTCATGCTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACCTGG
CGGCCGCTTG
* BAC-T7-KanStop-TetStop
(SEQ ID NO: 92)
ACAATTAATCATCGGCTCGAAGCTTGTTGACAATTAATCATCGGCATAGTATATCGGCATAGTATA
ATACGACAAGGTGAGGAACTAAACCATGGGATCGGCCATTGAACAAGATGGATTGCACGCAGGTT
CTCCGGCCGCTTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCGACCTGTCCG
GTGCCCTGAATGAACTGCAGGACGAGGCAGCGCGGCTATCGTAGCTGGCCACGACGGGCGTTCCT
TGCGCAGCTGTGCTCGACGTTGTCACTGAAGCGGGAAGGGACTGGCTGCTATTGGGCGAAGTGCC
GGGGCAGGATCTCCTGTCATCTCACCTTGCTCCTGCCGAGAAAGTATCCATCATGGCTGATGCAAT
GCGGCGGCTGCATACGCTTGATCCGGCTACCTGCCCATTCGACCACCAAGCGAAACATCGCATCGA
GCGAGCACGTACTCGGATGGAAGCCGGTCTTGTCGATCAGGATGATCTGGACGAAGAGCATCAGG
GGCTCGCGCCAGCCGAACTGTTCGCCAGGCTCAAGGCGCGCATGCCCGACGGCGATGATCTCGTC
GTGACCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGATATTGCT
GAAGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGGGGATCAATTCTCTAGAGCTTAAT
TAACGCAGCCTGAATGGCGAATAGGGATCCTTGACAGCTTATCATCGATAAGCTTTAATGCGGTAG
TTTATCACAGTTGCTAACGCAGTCAGGCACCGTGTATGAATAGTTCGACAAAGATCGCATTGGTAA
TTACGTTACTCGATGCCATGGGGATTGGCCTTATCATGCCAGTCTTGCCAACGTTATTACGTGAATT
TATTGCTTCGGAAGATATCGCTAACCACTTTGGCGTATTGCTTGCACTTTATGCGTTAATGCAGGTT
ATCTTTGCTCCTTAGCTTGGAAAAATGTCTGACCGATTTGGTCGGCGCCCAGTGCTGTTGTTGTCAT
TAATAGGCGCATCGCTGGATTACTTATTGCTGGCTTTTTCAAGTGCGCTTTGGATGCTGTATTTAGG
CCGTTTGCTTTCAGGGATCACAGGAGCTACTGGGGCTGTCGCGGCATCGGTCATTGCCGATACCAC
CTCAGCTTCTCAACGCGTGAAGTGGTTCGGTTGGTTAGGGGCAAGTTTTGGGCTTGGTTTAATAGC
GGGGCCTATTATTGGTGGTTTTGCAGGAGAGATTTCACCGCATAGTCCCTTTTTTATCGCTGCGTTG
CTAAATATTGTCACTTTCCTTGTGGTTATGTTTTGGTTCCGTGAAACCAAAAATACACGTGATAATA
CAGATACCGAAGTAGGGGTTGAGACGCAATCGAATTCGGTATACATCACTTTATTTAAAACGATGC
CCATTTTGTTGATTATTTATTTTTCAGCGCAATTGATAGGCCAAATTCCCGCAACGGTGTGGGTGCT
ATTTACCGAAAATCGTTTTGGATGGAATAGCATGATGGTTGGCTTTTCATTAGCGGGTCTTGGTCTT
TTACACTCAGTATTCCAAGCCTTTGTGGCAGGAAGAATAGCCACTAAATGGGGCGAAAAAACGGC
AGTACTGCTCGGATTTATTGCAGATAGTAGTGCATTTGCCTTTTTAGCGTTTATATCTGAAGGTTGG
TTAGTTTTCCCTGTTTTAATTTTATTGGCTGGTGGTGGGATCGCTTTACCTGCATTACAGGGAGTGA
TGTCTATCCAAACAAAGAGTCATCAGCAAGGTGCTTTACAGGGATTATTGGTGAGCCTTACCAATG
CAACCGGTGTTATTGGCCCATTACTGTTTGCTGTTATTTATAATCATTCACTACCAATTTGGGATGG
CTGGATTTGGATTATTGGTTTAGCGTTTTACTGTATTATTATCCTGCTATCGATGACCTTCATGTTAA
CCCCTCAAGCTCAGGGGAGTAAACAGGAGACAAGTGCTTAGTGATCCAATTCTTGAAGACGAAAG
GGCCTCGTGATACGCCTATTTTTATAGGTTAATGTCATGATAATAATGGTTTCTTAGACGTCAGGTG
GCACTTTTCGGGGAAATGTGCCCGGTTAACGTGCCGGCACGGCCTGGGTAACCAGGTATTTTGTCC
ACATAACCGTGCGCAAAATGTTGTGGATAAGCAGGACACAGCAGCAATCCACAGCAGGCATACAA
CCGCACACCGAGGTTACTCCGTTCTACAGGTTACGACGACATGTCAATACTTGCCCTTGACAGGCA
TTGATGGAATCGTAGTCTCACGCTGATAGTCTGATCGACAATACAAGTGGGACCGTGGTCCCAGAC
CGATAATCAGACCGACAACACGAGTGGGATCGTGGTCCCAGACTAATAATCAGACCGACGATACG
AGTGGGACCGTGGTCCCAGACTAATAATCAGACCGACGATACGAGTGGGACCGTGGTTCCAGACT
AATAATCAGACCGACGATACGAGTGGGACCGTGGTCCCAGACTAATAATCAGACCGACGATACGA
GTGGGACCATGGTCCCAGACTAATAATCAGACCGACGATACGAGTGGGACCGTGGTCCCAGTCTG
ATTATCAGACCGACGATACGAGTGGGACCGTGGTCCCAGACTAATAATCAGACCGACGATACGAG
TGGGACCGTGGTCCCAGACTAATAATCAGACCGACGATACGAGTGGGACCGTGGTCCCAGTCTGA
TTATCAGACCGACGATACAAGTGGAACAGTGGGCCCAGAGAGAATATTCAGGCCAGTTATGCTTT
CTGGCCTGTAACAAAGGACATTAAGTAAAGACAGATAAACGTAGACTAAAACGTGGTCGCATCAG
GGTGCTGGCTTTTCAAGTTCCTTAAGAATGGCCTCAATTTTCTCTATACACTCAGTTGGAACACGAG
ACCTGTCCAGGTTAAGCACCATTTTATCGCCCTTATACAATACTGTCGCTCCAGGAGCAAACTGAT
GTCGTGAGCTTAAACTAGTTCTTGATGCAGATGACGTTTTAAGCACAGAAGTTAAAAGAGTGATAA
CTTCTTCAGCTTCAAATATCACCCCAGCTTTTTTCTGCTCATGAAGGTTAGATGCCTGCTGCTTAAG
TAATTCCTCTTTATCTGTAAAGGCTTTTTGAAGTGCATCACCTGACCGGGCAGATAGTTCACCGGG
GTGAGAAAAAAGAGCAACAACTGATTTAGGCAATTTGGCGGTGTTGATACAGCGGGTAATAATCT
TACGTGAAATATTTTCCGCATCAGCCAGCGCAGAAATATTTCCAGCAAATTCATTCTGCAATCGGC
TTGCATAACGCTGACCACGTTCATAAGCACTTGTTGGGCGATAATCGTTACCCAATCTGGATAATG
CAGCCATCTGCTCATCATCCAGCTCGCCAACCAGAACACGATAATCACTTTCGGTAAGTGCAGCAG
CTTTACGACGGCGACTCCCATCGGCAATTTCTATGACACCAGATACTCTTCGACCGAACGCCGGTG
TCTGTTGACCAGTCAGTAGAAAAGAAGGGATGAGATCATCCAGTGCGTCCTCAGTAAGCAGCTCC
TGGTCACGTTCATTACCTGACCATACCCGAGAGGTCTTCTCAACACTATCACCCCGGAGCACTTCA
AGAGTAAACTTCACATCCCGACCACATACAGGCAAAGTAATGGCATTACCGCGAGCCATTACTCCT
ACGCGCGCAATTAACGAATCCACCATCGGGGCAGCTGGTGTCGATAACGAAGTATCTTCAACCGG
TTGAGTATTGAGCGTATGTTTTGGAATAACAGGCGCACGCTTCATTATCTAATCTCCCAGCGTGGTT
TAATCAGACGATCGAAAATTTCATTGCAGACAGGTTCCCAAATAGAAAGAGCATTTCTCCAGGCA
CCAGTTGAAGAGCGTTGATCAATGGCCTGTTCAAAAACAGTTCTCATCCGGATCTGACCTTTACCA
ACTTCATCCGTTTCACGTACAACATTTTTTAGAACCATGCTTCCCCAGGCATCCCGAATTTGCTCCT
CCATCCACGGGGACTGAGAGCCATTACTATTGCTGTATTTGGTAAGCAAAATACGTACATCAGGCT
CGAACCCTTTAAGATCAACGTTCTTGAGCAGATCACGAAGCATATCGAAAAACTGCAGTGCGGAG
GTGTAGTCAAACAACTCAGCAGGCGTGGGAACAATCAGCACATCAGCAGCACATACGACATTAAT
CGTGCCGATACCCAGGTTAGGCGCGCTGTCAATAACTATGACATCATAGTCATGAGCAACAGTTTC
AATGGCCAGTCGGAGCATCAGGTGTGGATCGGTGGGCAGTTTACCTTCATCAAATTTGCCCATTAA
CTCAGTTTCAATACGGTGCAGAGCCAGACAGGAAGGAATAATGTCAAGCCCCGGCCAGCAAGTGG
GCTTTATTGCATAAGTGACATCGTCCTTTTCCCCAAGATAGAAAGGCAGGAGAGTGTCTTCTGCAT
GAATATGAAGATCTGGTACCCATCCGTGATACATTGAGGCTGTTCCCTGGGGGTCGTTACCTTCCA
CGAGCAAAACACGTAGCCCCTTCAGAGCCAGATCCTGAGCAAGATGAACAGAAACTGAGGTTTTG
TAAACGCCACCTTTATGGGCAGCAACCCCGATCACCGGTGGAAATACGTCTTCAGCACGTCGCAAT
CGCGTACCAAACACATCACGCATATGATTAATTTGTTCAATTGTATAACCAACACGTTGCTCAACC
CGTCCTCGAATTTCCATATCCGGGTGCGGTAGTCGCCCTGCTTTCTCGGCATCTCTGATAGCCTGAG
AAGAAACCCCAACTAAATCCGCTGCTTCACCTATTCTCCAGCGCCGGGTTATTTTCCTCGCTTCCGG
GCTGTCATCATTAAACTGTGCAATGGCGATAGCCTTCGTCATTTCATGACCAGCGTTTATGCACTG
GTTAAGTGTTTCCATGAGTTTCATTCTGAACATCCTTTAATCATTGCTTTGCGTTTTTTTATTAAATC
TTGCAATTTACTGCAAAGCAACAACAAAATCGCAAAGTCATCAAAAAACCGCAAAGTTGTTTAAA
ATAAGAGCAACACTACAAAAGGAGATAAGAAGAGCACATACCTCAGTCACTTATTATCACTAGCG
CTCGCCGCAGCCGTGTAACCGAGCATAGCGAGCGAACTGGCGAGGAAGCAAAGAAGAACTGTTCT
GTCAGATAGCTCTTACGCTCAGCGCAAGAAGAAATATCCACCGTGGGAAAAACTCCAGGTAGAGG
TACACACGCGGATAGCCAATTCAGAGTAATAAACTGTGATAATCAACCCTCATCAATGATGACGA
ACTAACCCCCGATATCAGGTCACATGACGAAGGGAAAGAGAAGGAAATCAACTGTGACAAACTGC
CCTCAAATTTGGCTTCCTTAAAAATTACAGTTCAAAAAGTATGAGAAAATCCATGCAGGCTGAAGG
AAACAGCAAAACTGTGACAAATTACCCTCAGTAGGTCAGAACAAATGTGACGAACCACCCTCAAA
TCTGTGACAGATAACCCTCAGACTATCCTGTCGTCATGGAAGTGATATCGCGGAAGGAAAATACG
ATATGAGTCGTCTGGCGGCCTTTCTTTTTCTCAATGTATGAGAGGCGCATTGGAGTTCTGCTGTTGA
TCTCATTAACACAGACCTGCAGGAAGCGGCGGCGGAAGTCAGGCATACGCTGGTAACTTTGAGGC
AGCTGGTAACGCTCTATGATCCAGTCGATTTTCAGAGAGACGATGCCTGAGCCATCCGGCTTACGA
TACTGACACAGGGATTCGTATAAACGCATGGCATACGGATTGGTGATTTCTTTTGTTTCACTAAGC
CGAAACTGCGTAAACCGGTTCTGTAACCCGATAAAGAAGGGAATGAGATATGGGTTGATATGTAC
ACTGTAAAGCCCTCTGGATGGACTGTGCGCACGTTTGATAAACCAAGGAAAAGATTCATAGCCTTT
TTCATCGCCGGCATCCTCTTCAGGGCGATAAAAAACCACTTCCTTCCCCGCGAAACTCTTCAATGC
CTGCCGTATATCCTTACTGGCTTCCGCAGAGGTCAATCCGAATATTTCAGCATATTTAGCAACATG
GATCTCGCAGATACCGTCATGTTCCTGTAGGGTGCCATCAGATTTTCTGATCTGGTCAACGAACAG
ATACAGCATACGTTTTTGATCCCGGGAGAGACTATATGCCGCCTCAGTGAGGTCGTTTGACTGGAC
GATTCGCGGGCTATTTTTACGTTTCTTGTGATTGATAACCGCTGTTTCCGCCATGACAGATCCATGT
GAAGTGTGACAAGTTTTTAGATTGTCACACTAAATAAAAAAGAGTCAATAAGCAGGGATAACTTT
GTGAAAAAACAGCTTCTTCTGAGGGCAATTTGTCACAGGGTTAAGGGCAATTTGTCACAGACAGG
ACTGTCATTTGAGGGTGATTTGTCACACTGAAAGGGCAATTTGTCACAACACCTTCTCTAGAACCA
GCATGGATAAAGGCCTACAAGGCGCTCTAAAAAAGAAGATCTAAAAACTATAAAAAAAATAATTA
TAAAAATATCCCCGTGGATAAGTGGATAACCCCAAGGGAAGTTTTTTCAGGCATCGTGTGTAAGCA
GAATATATAAGTGCTGTTCCCTGGTGCTTCCTCGCTCACTCGAGCTACGGGGTCTGACGCTCAGTG
GAACGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCATAGTTGCCTGA
CTGCCCGTCGTGTAGATAACTACGATACGGGAGGGCTTACCATCTGGCCCCAGTGCTGCAATGATA
CCGCGGGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGTTACATGATC
CCCCATGTTGTGAAAAAAAGCGGTTAGCTCCTTCGGTCCTCCGATCGTTGTCAGAAGTAAGTTGGC
CGCAGTGTTATCACTCATGCTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCTCATC
ATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACCTGG
CGGCCGCCTTTCAGCAAAAAACCCCGCGAGACCCCCGAAGAGGCCCCGCGGGGTTATGCTAGGTC
GACGGAGCTCGAATTCTAATACGACTCACTATAGGGAGACCCAAGCTGGCTTG

### REFERENCES

1. Acar J. F. and Goldstein F. W., Genetic aspects and epidemiologic implications of resistance to trimethoprim, Rev. Infect. Dis. 1982 Mar.-Apr. 4; 4(2): 270-275.
2. Allen J. M., Simcha D. M., Ericson N. G., Alexander D. L., Marquette J. T., Van Biber B. P., Troll C. J., Karchin R., Bielas J. H., Loeb L. A., and Camps M., Roles of DNA polymerase I in leading and lagging-strand replication defined by a high-resolution mutation footprint of ColE1 plasmid replication, Nucleic Acids Res. 2011 May 26; 39(16): 7020-7033.
3. Alsøe L., Sarno A., Carracedo S., Domanska D., Dingler F., Lirussi L., SenGupta T., Tekin N. B., Jobert L., Alexandrov L. B., Galashevskaya A., Rada C., Sandve G. K., Rognes T., Krokan H. E., and Nilsen H., Uracil accumulation and mutagenesis dominated by cytosine deamination in CpG dinucleotides in mice lacking UNG and SMUG1, Sci. Rep. 2017 Aug. 3; 7(1): 7199.
4. Badran A. H. and Liu D. R., Development of potent in vivo mutagenesis plasmids with broad mutational spectra, Nat. Commun. 2015 Oct. 7; 6: 8425.
5. Badran A. H. and Liu D. R., In vivo continuous directed evolution, Curr. Opin. Chem. Biol. 2015 February; 24: 1-10.
6. Betts L., Xiang S., Short S. A., Wolfenden R., and Carter C. W. Jr., Cytidine deaminase. The 2.3 A crystal structure of an enzyme: transition-state analog complex, J. Mol. Biol. 1994 Jan. 14; 235(2): 635-56.
7. Bonner G., Lafer E. M., and Sousa R., Characterization of a set of T7 RNA polymerase active site mutants, J. Biol. Chem. 1994 Oct. 7; 269(40): 25120-28.
8. Camps M., Naukkarinen J., Johnson B. P., and Loeb L. A., Targeted gene evolution in Escherichia coli using a highly error-prone DNA polymerase I, Proc. Natl. Acad. Sci. U.S.A. 2003 Aug. 8; 100(17): 9727-9732.
9. Camsund D., Heidorn T., and Lindblad P., Design and analysis of LacI-repressed promoters and DNA-looping in a cyanobacterium, J. Biol. Eng. 2014 Jan. 27; 8(1): 4.
10. Chaudhuri J. and Alt F. W., Class-switch recombination: interplay of transcription, DNA deamination and DNA repair, Nat. Rev. Immunol. 2004 July; 4(7): 541-52.
11. Crook N., Abatemarco J., Sun J., Wagner J. M., Schmitz A., and Alper H. S., In vivo continuous evolution of genes and pathways in yeast, Nat. Commun. 2016 Oct. 17; 7: 13051.
12. Cupples C. G. and Miller J. H., A set of lacZ mutations in Escherichia coli that allow rapid detection of each of the six base substitutions, Proc. Natl. Acad. Sci. U.S.A. 1989 July; 86(14): 5345-49.
13. DiCarlo J. E., Conley A. J., Penttilä M., Jäntti J., Wang H. H., and Church G. M., Yeast oligo-mediated genome engineering (YOGE), ACS Synth. Biol. 2013 Dec. 20; 2(12): 741-749.
14. DeNizio J. E., Schutsky E. K., Berrios K. N., Liu M. Y., and Kohli R. M., Harnessing natural DNA modifying activities for editing of the genome and epigenome, Curr. Opin. Chem. Biol. 2018 Feb. 13; 45: 10-17.
15. Dower K. and Rosbash M., T7 RNA polymerase-directed transcripts are processed in yeast and link 3′ end formation to mRNA nuclear export, RNA. 2002 May; 8(5): 686-697.
16. Duncan B. K., Isolation of insertion, deletion, and nonsense mutations of the uracil-DNA glycosylase (ung) gene of Escherichia coli K-12, J. Bacteriol. 1985 November; 164(2): 689-95.
17. Durfee T., Nelson R., Baldwin S., Plunkett G. 3rd, Burland V., Mau B., Petrosino J. F., Qin X., Muzny D. M., Ayele M., Gibbs R. A., Csorgo B., Posfai G., Weinstock G. M., and Blattner F. R., The complete genome sequence of Escherichia coli DH10B: insights into the biology of a laboratory workhorse, J. Bacteriol. 2008 April; 190(7): 2597-606.
18. Garibyan L., Huang T., Kim M., Wolff E., Nguyen A., Nguyen T., Diep A., Hu K., Iverson A., Yang H., and Miller J. H., Use of the rpoB gene to determine the specificity of base substitution mutations on the Escherichia coli chromosome, DNA Repair. 2003 May; 2(5): 593-8.
19. Gaudelli N. M., Komor A. C., Rees H. A., Packer M. S., Badran A. H., Bryson D. I., Liu D. R., Programmable base editing of A*T to G*C in genomic DNA without DNA cleavage, Nature. 2017 Nov. 23; 551(7681): 464-71.
20. Geissmann Q., OpenCFU, a new free and open-source software to count cell colonies and other circular objects, PLoS One. 2013; 8(2): e54072.
21. Gerdes S. Y., Scholle M. D., Campbell J. W., Balazsi G., Ravasz E., Daugherty M. D., Somera A. L., Kyrpides N. C., Anderson I., Gelfand M. S., Bhattacharay A., Kapatral V., D'Souza M., Baev M. V., Grechkin Y., Mseeh F., Fonstein M. Y., Overbeek R., Barabasi A. L., Oltvai Z. N., and Osterman A. L., Experimental Determination and System Level Analysis of Essential Genes in Escherichia coli MG1655, J. Bacteriol. 2003 October; 185(19): 5673-84.
22. Glascock C. B. and Weickert M. J., Using chromosomal lacIQ1 to control expression of genes on high-copy-number plasmids in Escherichia coli, Gene. 1998 Nov. 26; 223(1-2): 221-31.
23. Greener A., Callahan M., and Jerpseth B., An efficient random mutagenesis technique using an E. coli mutator strain, Mol. Biotechnol. 1997 April; 7(2): 189-95.
24. Harris R. S., Petersen-Mahrt S. K., and Neuberger M. S., RNA Editing Enzyme APOBEC1 and Some of Its Homologs Can Act as DNA Mutators, Mol. Cell. 2002 November; 10(5): 1247-53.
25. Hecht A., Glasgow J., Jaschke P. R., Bawazer L. A., Munson M. S., Cochran J. R., Endy D., and Salit M., Measurements of translation initiation from all 64 codons in E. coli, Nucleic Acids Res. 2017 Apr. 20; 45(7): 3615-26.
26. Herrington M. B., MacRae T. J., Panagopoulos D., and Wong S. H., A mutation in the folA promoter delays adaptation to minimal medium by Escherichia coli K-12, J. Basic Microbiol. 2002; 42(3): 172.
27. Hess G. T., Fresard L., Han K., Lee C. H., Li A., Cimprich K. A., Montgomery S. B., and Bassik M. C., Directed evolution using dCas9-targeted somatic hypermutation in mammalian cells, Nat. Methods. 2016 December; 13(12): 1036-42.
28. Kim D., Lim K., Kim S. T., Yoon S. H., Kim K., Ryu S. M., and Kim J. S., Genome-wide target specificities of CRISPR RNA-guided programmable deaminases, Nat. Biotechnol. 2017 Apr. 10; 35(5): 475-480.
29. Komor A. C., Kim Y. B., Packer M. S., Zuris J. A., and Liu D. R., Programmable editing of a target base in genomic DNA without double-stranded DNA cleavage, Nature. 2016 May 19; 533(7603): 420-24.
30. Komor A. C., Zhao K. T., Packer M. S., Gaudelli N. M., Waterbury A. L., Koblan L. W., Kim Y. B., Badran A. H., and Liu D. R., Improved base excision repair inhibition and bacteriophage Mu Gam protein yields C:G-to-T:A base editors with higher efficiency and product purity, Sci. Adv. 2017 Aug. 30; 3(8): eaao4774.
31. Larkin M. A., Blackshields G., Brown N. P., Chenna R., McGettigan P. A., McWilliam H., Valentin F., Wallace I. M., Wilm A., Lopez R., Thompson J. D., Gibson T. J., and Higgins D. G., Clustal W and Clustal X version 2.0, Bioinformatics. 2007 Nov. 1; 23(21): 2947-48.
32. Li, H., Aligning sequence reads, clone sequences and assembly contigs with BWA-MEM., arXiv preprint arXiv. 16 Mar. 2013; 1303.3997.
33. Li H., A statistical framework for SNP calling, mutation discovery, association mapping and population genetical parameter estimation from sequencing data, Bioinformatics. 2011 Nov. 1; 27(21): 2987-93.
34. Li H., Handsaker B., Wysoker A., Fennell T., Ruan J., Homer N., Marth G., Abecasis G., Durbin R., and 1000 Genome Project Data Processing Subgroup, The Sequence Alignment/Map format and SAMtools, Bioinformatics. 2009 Aug. 15; 25(16): 2078-79.
35. Lieber A., Sandig V., and Strauss M., A mutant T7 phage promoter is specifically transcribed by T7-RNA polymerase in mammalian cells, Eur. J. Biochem., 1998 Oct. 1; 217(1): 387-94.
36. Lykke-Andersen J. and Christiansen J., The C-terminal carboxy group of T7 RNA polymerase ensures efficient magnesium ion-dependent catalysis, Nucleic Acids Res. 1998 Dec. 15; 26(24): 5630-35.
37. Ma Y., Zhang J., Yin W., Zhang Z., Song Y., and Chang X., Targeted AID-mediated mutagenesis (TAM) enables efficient genomic diversification in mammalian cells, Nat. Methods. 2016 December; 13(12): 1029-35.
38. Mairhofer J., Wittwer A., Cserjan-Puschmann M., and Striedner G., Preventing T7 RNA polymerase read-through transcription-A synthetic termination signal capable of improving bioprocess stability, ACS Synth. Biol. 2015 Mar. 20; 4(3): 265-73.
39. McBride K. E., Schaaf D. J., Daley M., and Stalker D. M., Controlled expression of plastid transgenes in plants based on a nuclear DNA-encoded and plastid-targeted T7 RNA polymerase, Proc. Natl. Acad. Sci. U.S.A. 1994 Jul. 19; 91(15): 7301-7305.
40. Miller A. W., Befort C., Kerr E. O., and Dunham M. J., Design and use of multiplexed chemostat arrays, J. Vis. Exp. 2013 Feb. 23; (72): e50262.
41. Nasvall J., Direct and Inverted Repeat stimulated excision (DIRex): Simple, single-step, and scar-free mutagenesis of bacterial genes, PLoS One. 2017 Aug. 30; 12(8): e0184126.
42. Navaratnam N., Bhattacharya S., Fujino T., Patel D., Jarmuz A. L., and Scott J., Evolutionary origins of apoB mRNA editing: Catalysis by a cytidine deaminase that has acquired a novel RNA-binding motif at its active site, Cell. 1995 Apr. 21; 81(2): 187-95.
43. Nilsen H., Rosewell I., Robins P., Skjelbred C. F., Andersen S., Slupphaug G., Daly G., Krokan H. E., Lindahl T., and Barnes D. E., Uracil-DNA glycosylase (UNG)-deficient mice reveal a primary role of the enzyme during DNA replication, Mol. Cell. 2000 June; 5(6): 1059-1065.
44. Nilsson A. I., Berg O. G., Aspevall O., Kahlmeter G., and Andersson D. I., Biological Costs and Mechanisms of Fosfomycin Resistance in Escherichia coli, Antimicrob. Agents Chemother. 2003 September; 47(9): 2850-58.
45. Nishida K., Arazoe T., Yachie N., Banno S., Kakimoto M., Tabata M., Mochizuki M., Miyabe A., Araki M., Hara K. Y., Shimatani Z., and Kondo A., Targeted nucleotide editing using hybrid prokaryotic and vertebrate adaptive immune systems, Science. 2016 Sep. 16; 353(6305): pii: aaf8729.
46. Petersen-Mahrt S. K., Harris R. S., and Neuberger M. S., AID mutates E. coli suggesting a DNA deamination mechanism for antibody diversification, Nature. 2002 Jul. 4; 418(6893): 99-103.
47. Pratt L. A. and Kolter R., Genetic analysis of Escherichia coli biofilm formation: roles of flagella, motility, chemotaxis and type I pili, Mol. Microbiol. 1998 October; 30(2): 285-93.
48. Prigent-Combaret C., Prensier G., Le Thi T. T., Vidal O., Lejeune P., and Dorel C., Developmental pathway for biofilm formation in curli-producing Escherichia coli strains: role of flagella, curli and colanic acid, Environ. Microbiol. 2000 August; 2(4): 450-64.
49. Qiao Q., Wang L., Meng F. L., Hwang J. K., Alt F. W., and Wu H., AID Recognizes Structured DNA for Class Switch Recombination, Mol. Cell. 2017 Aug. 3; 67(3): 361-73.
50. Ramiro A. R., Stavropoulos P., Jankovic M., and Nussenzweig M. C., Transcription enhances AID-mediated cytidine deamination by exposing single-stranded DNA on the nontemplate strand, Nat. Immunol. 2003 May; 4(5): 452-56.
51. Ravikumar A., Arrieta A., and Liu C. C., An orthogonal DNA replication system in yeast, Nat. Chem. Biol. 2014 Feb. 2; 10(3): 175-177.
52. Rong M., Durbin R. K., and McAllister W. T., Template strand switching by T7 RNA polymerase, J. Biol. Chem. 1998 Apr. 24; 273(17): 10253-60.
53. Schaefer J., Jovanovic G., Kotta-Loizou I., and Buck M., Single-step method for beta-galactosidase assays in Escherichia coli using a 96-well microplate reader, Anal. Biochem. 2016 Mar. 29; 503: 56-57.
54. Serrano-Heras G., Ruiz-Masó J. A., del Solar G., Espinosa M., Bravo A., and Salas M., Protein p56 from the Bacillus subtilis phage phi29 inhibits DNA-binding ability of uracil-DNA glycosylase, Nucleic Acids Res. 2007 Aug. 13; 35(16): 5393-5401.
55. Tessman I., Ishiwa H., and Kumar S., Mutagenic Effects of Hydroxylamine in vivo. Science. 1965 Apr. 23; 148(3669): 507-8.
56. Thiel V., Herold J., Schelle B., and Siddell S. G., Infectious RNA transcribed in vitro from a cDNA copy of the human coronavirus genome cloned in vaccinia virus, J. Gen. Virol. 2001 June; 82(6): 1273-81.
57. Tizei P. A., Csibra E., Tones L., and Pinheiro V. B., Selection platforms for directed evolution in synthetic biology, Biochem. Soc. Trans. 2016 Aug. 15; 44(4): 1165-1175
58. Wang T., Birsoy K., Hughes N. W., Krupczak K. M., Post Y., Wei J. J., Lander E. S., and Sabatini D. M., Identification and characterization of essential genes in the human genome, Science. 2015 Nov. 27; 350(6264): 1096-101.
59. Wang H., Bian X., Xia L., Ding X., Muller R., Zhang Y., Fu J., and Stewart A. F., Improved seamless mutagenesis by recombineering using ccdB for counterselection, Nucleic Acids Res. 2014 March; 42(5): e37.
60. Weinstock M. T., Hesek E. D., Wilson C. M., and Gibson D. G., Vibrio natriegens as a fast-growing host for molecular biology, Nat. Methods. 2016 Aug. 29; 13(10): 849-851.
61. Wong T. S., Zhurina D., and Schwaneberg U., The Diversity Challenge in Directed Protein Evolution, Comb. Chem. High Throughput Screen. 2006 May; 9(4): 271-88.
62. Wycuff D. R. and Matthews K. S., Generation of an AraC-araBAD promoter-regulated T7 expression system, Anal. Biochem. 2000 Jan. 1; 277(1): 67-73.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A nucleobase-editing fusion protein comprising a processive polynucleic acid-binding protein fused to a nucleobase-editing enzyme which is capable of altering nucleobases in a pre-existing polynucleic acid sequence.

2. The nucleobase-editing fusion protein of claim 1, wherein:
the processive polynucleic acid-binding protein of the nucleobase-editing fusion protein comprises the amino acid sequence of an RNA polymerase, a DNA polymerase, a DNA methyltransferase, a DNA glycosylase, or a DNA helicase;
the nucleobase-editing enzyme comprises the amino acid sequence of an Apobec protein, a TadA protein, an AMPD protein, a CDA protein, an ADAT protein, an ADAR protein, or a GDA protein; or
a combination thereof.

3. The nucleobase-editing fusion protein of claim 1, wherein the processive polynucleic acid-binding protein of the nucleobase-editing fusion protein comprises the amino acid sequence of T7 RNA polymerase or a functional variant thereof.

4. (canceled)

5. The nucleobase-editing fusion protein of claim 1, wherein the nucleobase-editing enzyme comprises the amino acid sequence of an Apobec protein, optionally wherein the Apobec protein is rApobec1 or a functional variant thereof.

6. (canceled)

7. The nucleobase-editing fusion protein of claim 1, wherein the nucleobase-editing enzyme comprises the amino acid sequence of a TadA protein, optionally wherein the TadA protein is E. coli TadA comprising an A106V mutation and/or a D108N mutation or a protein homolog comprising a homologous mutation(s).

8. (canceled)

9. A method of performing dynamic targeted hypermutation comprising contacting at least one polynucleic acid with at least one non-naturally occurring nucleobase-editing fusion protein, wherein:
a. each of the at least one non-naturally occurring nucleobase-editing fusion proteins comprises a processive polynucleic acid-binding protein fused to a nucleobase-editing enzyme;
b. each of the at least one polynucleic acid comprises a target region; and
c. the contacting of the at least one polynucleic acid with the at least one non-naturally occurring nucleobase-editing fusion protein generates mutations at a rate exceeding background mutation rates only in the target region of the at least one polynucleic acid of (b), wherein the background mutation rate of the at least one polynucleic acid of (b) is determined in the absence of the non-naturally occurring nucleobase-editing fusion protein.

10. The method of claim 9, wherein:
the processive polynucleic acid-binding protein of the nucleobase-editing fusion protein comprises the amino acid sequence of an RNA polymerase, a DNA polymerase, a DNA methyltransferase, a DNA glycosylase, or a DNA helicase;
the nucleobase-editing enzyme comprises the amino acid sequence of an Apobec protein, a TadA protein, an AMPD protein, a CDA protein, an ADAT protein, an ADAR protein, or a GDA protein; or
a combination thereof.

11. The method of claim 9, wherein the processive polynucleic acid-binding protein of at least one of the at least one non-naturally occurring nucleobase-editing fusion proteins comprises the amino acid sequence of T7 RNA polymerase or a functional variant thereof.

12. (canceled)

13. The method of claim 9, wherein the nucleobase-editing enzyme of at least one of the at least one non-naturally occurring nucleobase-editing fusion proteins comprises:
the amino acid sequence of an Apobec protein, optionally wherein the Apobec protein is rApobec1 or a functional variant thereof;
the amino acid sequence of a TadA protein, optionally wherein the TadA protein is E. coli TadA comprising an A106V mutation and/or a D108N mutation or a protein homolog comprising a homologous mutation(s); or
a combination thereof.

14.-16. (canceled)

17. The method of claim 9, wherein each of the at least one polynucleic acid comprises, from 5′ to 3′: a promoter region that is bound by at least one of the at least one non-naturally occurring nucleobase-editing fusion proteins in a sequence-specific manner; the target region; and a terminator region comprising a terminator array.

18. The method of claim 17, wherein:
the terminator array comprises four or more terminators, optionally four or more T7 UUCG terminators;
the promoter region of at least one of the at least one polynucleic acids comprises the sequence of SEQ ID NO: 21, and/or SEQ ID NO: 22, SEQ ID NO: 23;
the contacting of the at least one polynucleic acid with the at least one non-naturally occurring nucleobase-editing fusion protein occurs in a living cell; or
a combination thereof.

19.-20. (canceled)

21. The method of claim 18, wherein:
at least one of the at least one non-naturally occurring nucleobase-editing fusion proteins is encoded on a plasmid, wherein the plasmid has copy number of less than 10;
at least one of the at least one non-naturally occurring nucleobase-editing fusion proteins is conditionally expressed in the living cell; or
a combination thereof.

22. (canceled)

23. The method of claim 18, wherein the living cell contains a modified genome comprising:
a. an integration of a polynucleic acid sequence encoding for and driving the expression of at least one non-naturally occurring nucleobase-editing fusion protein; and/or
b. an integration of a polynucleic sequence comprising, from 5′ to 3′: a promoter region that is bound by at least one of the at least one non-naturally occurring nucleobase-editing fusion proteins in a sequence-specific manner; the target region; and a terminator region comprising a terminator array.

24. The method of claim 18, wherein the living cell contains a modified genome and a plasmid that facilitates expression of a T7 inhibitor, optionally wherein the T7 inhibitor is T7 lysozyme, wherein the modified genome of the living cell comprises:
a. an integration of a polynucleic acid sequence encoding for and driving the expression of the non-naturally occurring nucleobase-editing fusion protein, wherein the sequence driving the expression of the fusion protein comprises a sequence bound by LacI repressor that inhibits transcription of the fusion protein when LacI is bound; and/or
b. a deletion of genomic sequence encoding for uracil deglycosylase.

25. (canceled)

26. The method of claim 21, wherein the living cell is treated to increase the expression and/or activity of the uracil deglycoslyase inhibitor, ugi.

27. A kit for performing dynamic targeted hypermutation comprising:
a. a polypeptide comprising the amino acid sequence of a non-naturally occurring nucleobase-editing fusion protein comprising a processive polynucleic acid-binding protein fused to a nucleobase-editing enzyme or a polynucleic acid sequence encoding for and driving the expression of said polypeptide; and
b. a polynucleic acid sequence comprising, from 5′ to 3′: a promoter region that is bound by the non-naturally occurring nucleobase-editing fusion protein of (a) in a sequence-specific manner; a cloning site; and a terminator region comprising a terminator array.

28. (canceled)

29. The kit of claim 27, wherein:
the processive polynucleic acid-binding protein of the nucleobase-editing fusion protein comprises the amino acid sequence of an RNA polymerase, a DNA polymerase, a DNA methyltransferase, a DNA glycosylase, or a DNA helicase;
the nucleobase-editing enzyme comprises the amino acid sequence of an Apobec protein, a TadA protein, an AMPD protein, a CDA protein, an ADAT protein, an ADAR protein, or a GDA protein; or
a combination thereof.

30. The kit of claim 27, wherein the processive polynucleic acid-binding protein of the non-naturally occurring nucleobase-editing fusion protein comprises the amino acid sequence of T7 RNA polymerase or a functional variant thereof.

31. (canceled)

32. The kit of claim 27, wherein the nucleobase-editing enzyme of the nucleobase-editing fusion protein comprises:
the amino acid sequence of an Apobec protein, optionally wherein the Apobec protein is rApobec1 or a functional variant thereof;
the amino acid sequence of a TadA protein, optionally wherein the TadA protein is E. coli TadA comprising an A106V mutation and/or a D108N mutation or a protein homolog comprising a homologous mutation(s); or
a combination thereof.

33.-35. (canceled)

36. The kit of claim 27, wherein;
the terminator array comprises four or more terminators, optionally four or more T7 UUCG terminators;
the promoter region comprises the sequence of SEQ ID NO: 21, SEQ ID NO: 22, and/or SEQ ID NO: 23; or
a combination thereof.

37. (canceled)


SEQUENCE LISTING (101 sequences)

SEQ ID NO: 1 - DNA, 35 bp, Artificial Sequence
  Synthetic Polynucleotide
ttgacagctagctcagtcctaggtataatgctagc

SEQ ID NO: 2 - DNA, 35 bp, Artificial Sequence
  Synthetic Polynucleotide
ttgacggctagctcagtcctaggtacagtgctagc

SEQ ID NO: 3 - DNA, 35 bp, Artificial Sequence
  Synthetic Polynucleotide
tttacagctagctcagtcctaggtattatgctagc

SEQ ID NO: 4 - DNA, 35 bp, Artificial Sequence
  Synthetic Polynucleotide
ttgacagctagctcagtcctaggtactgtgctagc

SEQ ID NO: 5 - DNA, 35 bp, Artificial Sequence
  Synthetic Polynucleotide
ctgatagctagctcagtcctagggattatgctagc

SEQ ID NO: 6 - DNA, 35 bp, Artificial Sequence
  Synthetic Polynucleotide
ttgacagctagctcagtcctaggtattgtgctagc

SEQ ID NO: 7 - DNA, 35 bp, Artificial Sequence
  Synthetic Polynucleotide
tttacggctagctcagtcctaggtactatgctagc

SEQ ID NO: 8 - DNA, 35 bp, Artificial Sequence
  Synthetic Polynucleotide
tttacggctagctcagtcctaggtatagtgctagc

SEQ ID NO: 9 - DNA, 35 bp, Artificial Sequence
  Synthetic Polynucleotide
tttacggctagctcagccctaggtattatgctagc

SEQ ID NO: 10 - DNA, 35 bp, Artificial Sequence
  Synthetic Polynucleotide
ctgacagctagctcagtcctaggtataatgctagc

SEQ ID NO: 11 - DNA, 35 bp, Artificial Sequence
  Synthetic Polynucleotide
tttacagctagctcagtcctagggactgtgctagc

SEQ ID NO: 12 - DNA, 35 bp, Artificial Sequence
  Synthetic Polynucleotide
tttacggctagctcagtcctaggtacaatgctagc

SEQ ID NO: 13 - DNA, 35 bp, Artificial Sequence
  Synthetic Polynucleotide
ttgacggctagctcagtcctaggtatagtgctagc

SEQ ID NO: 14 - DNA, 35 bp, Artificial Sequence; identical to SEQ ID NO: 5
  Synthetic Polynucleotide

SEQ ID NO: 15 - DNA, 35 bp, Artificial Sequence
  Synthetic Polynucleotide
ctgatggctagctcagtcctagggattatgctagc

SEQ ID NO: 16 - DNA, 35 bp, Artificial Sequence
  Synthetic Polynucleotide
tttatggctagctcagtcctaggtacaatgctagc

SEQ ID NO: 17 - DNA, 35 bp, Artificial Sequence
  Synthetic Polynucleotide
tttatagctagctcagcccttggtacaatgctagc

SEQ ID NO: 18 - DNA, 35 bp, Artificial Sequence
  Synthetic Polynucleotide
ttgacagctagctcagtcctagggactatgctagc

SEQ ID NO: 19 - DNA, 35 bp, Artificial Sequence
  Synthetic Polynucleotide
ttgacagctagctcagtcctagggattgtgctagc

SEQ ID NO: 20 - DNA, 35 bp, Artificial Sequence
  Synthetic Polynucleotide
ttgacggctagctcagtcctaggtattgtgctagc

SEQ ID NO: 21 - DNA, 19 bp, Artificial Sequence
  Synthetic Polynucleotide
taatacgactcactatagg

SEQ ID NO: 22 - DNA, 19 bp, Artificial Sequence
  Synthetic Polynucleotide
aattaaccctcactaaagg

SEQ ID NO: 23 - DNA, 19 bp, Artificial Sequence
  Synthetic Polynucleotide
atttaggtgacactataga

SEQ ID NO: 24 - DNA, 72 bp, Artificial Sequence
  Synthetic Polynucleotide
aaagagtgtttatttgtgagcggataacaattacaattagattcaattgtgagcggataacaatttcacaca

SEQ ID NO: 25 - DNA, 5 bp, Artificial Sequence
  Synthetic Polynucleotide
ttgac

SEQ ID NO: 26 - DNA, 5 bp, Artificial Sequence
  Synthetic Polynucleotide
tttat

SEQ ID NO: 27 - DNA, 6 bp, Artificial Sequence
  Synthetic Polynucleotide
gatact

SEQ ID NO: 28 - DNA, 6 bp, Artificial Sequence
  Synthetic Polynucleotide
tacaat

SEQ ID NO: 29 - DNA, 75 bp, Artificial Sequence
  Synthetic Polynucleotide
gcagttaagctaggcggattgaagattcgcaggagagcgagatggctaacccctcatcagtgccaacatagtaag

SEQ ID NO: 30 - DNA, 67 bp, Artificial Sequence
  Synthetic Polynucleotide
agccgggtggcaactctgccatccggcatttccccgcaaatttactcactccgctcattaggcgggc

SEQ ID NO: 31 - DNA, 100 bp, Artificial Sequence
  Synthetic Polynucleotide
gcagttaagctaggcggattgaagattcgcaggagagcgagatggctaacagtgagtaaatttgcggggaaatgccggatggcagagttgccacccggct

SEQ ID NO: 32 - DNA, 100 bp, Artificial Sequence
  Synthetic Polynucleotide
agccgggtggcaactctgccatccggcatttccccgcaaatttactcactgttagccatctcgctctcctgcgaatcttcaatccgcctagcttaactgc

SEQ ID NO: 33 - DNA, 75 bp, Artificial Sequence
  Synthetic Polynucleotide
cgttactggtttcacattcaccaccctgaattgactctcttccgggcgctccctcatcagtgccaacatagtaag

SEQ ID NO: 34 - DNA, 67 bp, Artificial Sequence
  Synthetic Polynucleotide
tggtggccggaaggcgaagcggcatgcatttacgttgacaccatcgaatgccgctcattaggcgggc

SEQ ID NO: 35 - DNA, 99 bp, Artificial Sequence
  Synthetic Polynucleotide
attcaccaccctgaattgactctcttccgggcgctcattatacgagccgatgattaattgtcaacattcgatggtgtcaacgtaaatgcatgccgcttc

SEQ ID NO: 36 - DNA, 99 bp, Artificial Sequence
  Synthetic Polynucleotide
gaagcggcatgcatttacgttgacaccatcgaatgttgacaattaatcatcggctcgtataatgagcgcccggaagagagtcaattcagggtggtgaat

SEQ ID NO: 37 - DNA, 96 bp, Artificial Sequence
  Synthetic Polynucleotide
tttcgcttaaacagtaaaatgccggatgataattccggcttttttatctgtttgttgaaatatcggaattaaaaaagaattcaaaaaaaagcccgc

SEQ ID NO: 38 - DNA, 96 bp, Artificial Sequence
  Synthetic Polynucleotide
gcagcagaccattctctccagattcatcttatgctcgatatttcaacaaacagataaaaaagccggaattaaaaaagaattcaaaaaaaagcccgc

SEQ ID NO: 39 - DNA, 96 bp, Artificial Sequence
  Synthetic Polynucleotide
cttcatcaaaaaatgtctgataaaaatcgcttatatccatgctcacgctggacatcatccttccagaattaaaaaagaattcaaaaaaaagcccgc

SEQ ID NO: 40 - DNA, 96 bp, Artificial Sequence
  Synthetic Polynucleotide
cgcctgacgactgaacatcctgtcatggtcaacagtggaaggatgatgtccagcgtgagcatggagaattaaaaaagaattcaaaaaaaagcccgc

SEQ ID NO: 41 - DNA, 21 bp, Artificial Sequence
  Synthetic Polynucleotide
gctcgacgttgtcactgaagc

SEQ ID NO: 42 - DNA, 15 bp, Artificial Sequence
  Synthetic Polynucleotide
cgccgtcgggcatgc

SEQ ID NO: 43 - DNA, 67 bp, Artificial Sequence
  Synthetic Polynucleotide
gtcgtcactctatctggcgtcacacctctcagaacaccaacaaacacgttccgctcattaggcgggc

SEQ ID NO: 44 - DNA, 74 bp, Artificial Sequence
  Synthetic Polynucleotide
ttcgggcagaagtaacgttcggtggtgaatttttcgatgaagttaacttccctcatcagtgccaacatagtaag

SEQ ID NO: 45 - DNA, 100 bp, Artificial Sequence
  Synthetic Polynucleotide
gtcgtcactctatctggcgtcacacctctcagaacaccaacaaacacgttcaagttaacttcatcgaaaaattcaccaccgaacgttacttctgcccgaa

SEQ ID NO: 46 - DNA, 100 bp, Artificial Sequence
  Synthetic Polynucleotide
ttcgggcagaagtaacgttcggtggtgaatttttcgatgaagttaacttgaacgtgtttgttggtgttctgagaggtgtgacgccagatagagtgacgac

SEQ ID NO: 47 - DNA, 75 bp, Artificial Sequence
  Synthetic Polynucleotide
cagcagcagaacgcccggcacgtgctctgccagttgttcaatggcctgatccctcatcagtgccaacatagtaag

SEQ ID NO: 48 - DNA, 67 bp, Artificial Sequence
  Synthetic Polynucleotide
tgagcaggcaatcagcagttgataaccccgttgccgcgcctggcgttcaaccgctcattaggcgggc

SEQ ID NO: 49 - DNA, 74 bp, Artificial Sequence
  Synthetic Polynucleotide
cagcagcagaacgcccggcacgtgctctgccagttgttcaatggcctgattcgagtttatggctagctcagtcc

SEQ ID NO: 50 - DNA, 73 bp, Artificial Sequence
  Synthetic Polynucleotide
tgagcaggcaatcagcagttgataaccccgttgccgcgcctggcgttcaactgaaaatcttctctcatccgcc

SEQ ID NO: 51 - DNA, 67 bp, Artificial Sequence
  Synthetic Polynucleotide
gcagaacgcccggcacgtgctctgccagttgttcaatggcctgattcgagccgctcattaggcgggc

SEQ ID NO: 52 - DNA, 75 bp, Artificial Sequence
  Synthetic Polynucleotide
cgacgacgcagggtcgggtcaaccgcaaccggaccggtttcagaagacatccctcatcagtgccaacatagtaag

SEQ ID NO: 53 - DNA, 16 bp, Artificial Sequence
  Synthetic Polynucleotide
gcagaacgcccggcac

SEQ ID NO: 54 - DNA, 15 bp, Artificial Sequence
  Synthetic Polynucleotide
cgacgcagggtcggg

SEQ ID NO: 55 - DNA, 273 bp, Artificial Sequence
  Synthetic Polynucleotide
gcagaacgcccggcacgtgctctgccagttgttcaatggcctgattcgagaaagagtgtttatttgtgagcggataacaattacaattagattcaattgtgagcggataacaatttcacacaggctagcgaattcgagctccctctagaaataattttgtttaactttaagaaggagatataccatgggcagcagctacccatacgacgtaccagattacgctatgtcttctgaaaccggtccggttgcggttgacccgaccctgcgtcgtcg

SEQ ID NO: 56 - DNA, 95 bp, Artificial Sequence
  Synthetic Polynucleotide
ctagccagcttgggtctccctaggtcgagctccgtcgacctagcataaccccgcggggcctcttcgggggtctcgcggggttttttgctgaaagg

SEQ ID NO: 57 - DNA, 95 bp, Artificial Sequence
  Synthetic Polynucleotide
gatccctttcagcaaaaaaccccgcgagacccccgaagaggccccgcggggttatgctaggtcgacggagctcgacctagggagacccaagctgg

SEQ ID NO: 58 - DNA, 97 bp, Artificial Sequence
  Synthetic Polynucleotide
aaaaaaaagcttgttgacaattaatcatcggcatagtatatcggcatagtataatacgacaaggtgaggaactaaaccacgggatcggccattgaac

SEQ ID NO: 59 - DNA, 38 bp, Artificial Sequence
  Synthetic Polynucleotide
aaaaaattaattaagctctagagaattgatcccctcag

SEQ ID NO: 60 - DNA, 29 bp, Artificial Sequence
  Synthetic Polynucleotide
ttgacaattaatcatcggctcgaagcttg

SEQ ID NO: 61 - DNA, 30 bp, Artificial Sequence
  Synthetic Polynucleotide
aaaaaaggatccttcgccattcaggctgcg

SEQ ID NO: 62 - DNA, 8116 bp, Artificial Sequence
  Synthetic Polynucleotide
acaattaatcatcggctcgaagcttgttgacaattaatcatcggcatagtatatcggcatagtataatacgacaaggtgaggaactaaaccacgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagcttaattaacgcagcctgaatggcgaatagggatccttgacagcttatcatcgataagctttaatgcggtagtttatcacagttgctaacgcagtcaggcaccgtgtacgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagtgatccaattcttgaagacgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcttg

SEQ ID NO: 63 - DNA, 10023 bp, Artificial Sequence
  Synthetic Polynucleotide
acgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcctttcagcaaaaaaccccgcgagacccccgaagaggccccgcggggttatgctaggtcgacggagctcgaattctaatacgactcactatagggagacccaagctggcttgacaattaatcatcggctcgaagcttgttgacaattaatcatcggcatagtatatcggcatagtataatacgacaaggtgaggaactaaaccacgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagcttaattaacgcagcctgaatggcgaatagaagtttaaacgctagattggacaatggcgagcctagtctcccacggcgatcttgccgcccttcttggccttaatgagaatctcgcggatcttgcgggcgtccaacttgccggtcagtcctttaggcacctcgtccacgaacacaacaccaccgcgcagcttcttggcggttgtaacctggctggccacatagtccacgatctccttctcggtcatggttttaccgtgttccagcacgacgactgcggcgggcagctcgccggcatcgtcgtcgggcaggccggcgaccccggcgtcgaagatgttggggtgttgcagcaggatgctctccagttcggctggggctacctggtagcccttgtatttgatcaggctcttcagccggtccacgatgaagaagtgctcgtcctcgtcccagtaggcgatgtcgccgctgtgcagccagccgtccttgtcgatgagagcgtttgtagcctcggggttgttaacgtagccgctcatgatcatggggccacggacgcacagctcgccgcgctggttcacacccagtgtcttaccggtgtccaagtccaccaccttagcctcgaagaagggcaccaccttgcctactgcgccaggcttgtcgtccccttcgggggtgatcagaatggcgctggttgtttctgtcaggccgtagccctggcggatgcctggtaggtggaagcgtttggccacggcctcacctacctccttgctgagcggcgccccgccgctggcgatctcgtgcaagttgcttaggtcgtacttgtcgatgagagtgctcttagcgaagaagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgatctggttgccgaagatggggtcgcgggcatgactgaatcggacacaagcggtgcggtgcggtagggctacgcccttgggcaatccggtactgccactactgttcatgatcagggcgatggttttgtcccggtcgaagctctcgggcacgaagtcgtactcgttgaagccgggtggcaaatgggaagtcacgaaggtgtacatgctttggaagccctggtagtcggtcttgctatccatgatgatgatcttttgtatgatcggtagcttcttttgcacgttgaggatcttttgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcaagctattctcgctgcacaccacgatccgatggtttgtattcagcccatagcgcttcatagcttctgccagccgaacgctcatctcgaagtactcggcgtaggtaatgtccacctcgatatgtgcgtcggtaaaggcgatggtgccgggcaccagggcgtagcgcttcatggctttgtgcagctgctcgccggcggtcccgtcttcgagtgggtagaatggcgctgggcccttcttaatgtttttggcatcttccatggtggtgaattccaccacactggactagtggatcctagggatgttttggctccatatgatcactacaaagacaccagaacaggtgttgtagttggaccagattccaaccgatccttgacagcttatcatcgataagctttaatgcggtagtttatcacagttgctaacgcagtcaggcaccgtgtacgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagtgatccaattcttgaag

SEQ ID NO: 64 - DNA, 8882 bp, Artificial Sequence
  Synthetic Polynucleotide
acgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcctttcagcaaaaaaccccgcgagacccccgaagaggccccgcggggttatgctaggtcgacggagctcgaattctaatacgactcactatagggagacccaagctggcttgacaattaatcatcggctcgaagcttgttgacaattaatcatcggcatagtatatcggcatagtataatacgacaaggtgaggaactaaaccacgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagcttaattaacgcagcctgaatggcgaatagaagtttaaacgctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctaggaagtttaaacgctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctaggatatattgatccttgacagcttatcatcgataagctttaatgcggtagtttatcacagttgctaacgcagtcaggcaccgtgtacgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagtgatccaattcttgaag

SEQ ID NO: 65 - DNA, 7059 bp, Artificial Sequence
  Synthetic Polynucleotide
acgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcctttcagcaaaaaaccccgcgagacccccgaagaggccccgcggggttatgctaggtcgacggagctcgaattctaatacgactcactatagggagacccaagctggcttgacaattaatcatcggctcgaagcttgttgacaattaatcatcggcatagtatatcggcatagtataatacgacaaggtgaggaactaaaccacgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagcttaattaacgcagcctgaatggcgaatagaagtttaaacgctagccagcttgggtctccctatagtgagtcgtattatcgagctccgtcgacctagcataaccccgcggggcctcttcgggggtctcgcggggttttttgctgaaagggatccaattcttgaag

SEQ ID NO: 66 - DNA, 2191 bp, Artificial Sequence
  Synthetic Polynucleotide
cctcccacacataaccaggaggtcagattatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataacccagaagcttagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaatggttcgcccgcctaatgagcgggcttttttttgaattcttttttaattcgatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggatccccctcatcagtgccaacatagtaagccagtatacactccgctagcgcggccgcctcgagtttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtaccgttagc

SEQ ID NO: 67 - DNA, 2999 bp, Artificial Sequence
  Synthetic Polynucleotide
cacataaccaggaggtcagattatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataacccagaagcttagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaatggttcgcccgcctaatgagcgggcttttttttgaattcttttttaattcgatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggatccgaattaaaaaagaattcaaaaaaaagcccgctcattaggcgggcgaaccaaccggtttaggcgaccacaggtttgcgtgcaatggaaatttcacactgctcaaccgaagtgtaatccttgttgtgattggttacatccagtttgcggtcaacatagtgataccctggcatcttcacaggcttctttgccttgtaagtagttttaaattcacacaaatagtgaccgcctccttctaacttcagagccataaagttgtttcctagcagcattccatctcgtgcaaagagacgctcagtgttgggttcccagccctgtgtcttcttctgcatgacaggtccattgggaggaaagttcaaaccagagaacttgacatggtagatgaaacagttgccttggatgctggaatcattgctgacagtacacactgcaccatcttcaaagttcatgatcctctcccatgtatagccctccgggaatgactgctttacatagtcagggatgtcttcagggtacttggtgaatggtatgcttccgtactgacactgtggtgataaaatatcccaagcaaatggcagaggtccgcccttggtgacagtgagctttaccgtctgctccccctcgtagggcttaccttttccatcgccttcgacctcaaagtagtgtccattgaccgtgcctgacatataaaccttgtaggtcatttgtttagcgatcacactcatctagtatttctcctctttaattactagatccacacattataggtacaaaaagacattatacgagccggaagcataaagtgtaaaggtacccatcagtgccaacatagtaagccagtatacactccgctagcgcggccgcctcgagtttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtaccgttagccctccca

SEQ ID NO: 68 - DNA, 2578 bp, Artificial Sequence
  Synthetic Polynucleotide
cacataaccaggaggtcagattatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataacccagaagcttagcaaaagctaaaaccaggagctatttaggtacctttacactttatgcttccggctcgtataatgtctttttgtacctataatgtgtggatctagtaattaaagaggagaaatactagatgagtgtgatcgctaaacaaatgacctacaaggtttatatgtcaggcacggtcaatggacactactttgaggtcgaaggcgatggaaaaggtaagccctacgagggggagcagacggtaaagctcactgtcaccaagggcggacctctgccatttgcttgggatattttatcaccacagtgtcagtacggaagcataccattcaccaagtaccctgaagacatccctgactatgtaaagcagtcattcccggagggctatacatgggagaggatcatgaactttgaagatggtgcagtgtgtactgtcagcaatgattccagcatccaaggcaactgtttcatctaccatgtcaagttctctggtttgaactttcctcccaatggacctgtcatgcagaagaagacacagggctgggaacccaacactgagcgtctctttgcacgagatggaatgctgctaggaaacaactttatggctctgaagttagaaggaggcggtcactatttgtgtgaatttaaaactacttacaaggcaaagaagcctgtgaagatgccagggtatcactatgttgaccgcaaactggatgtaaccaatcacaacaaggattacacttcggttgagcagtgtgaaatttccattgcacgcaaacctgtggtcgcctaaaccggttggttcgcccgcctaatgagcgggcttttttttgaattcttttttaattcgatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggatccccctcatcagtgccaacatagtaagccagtatacactccgctagcgcggccgcctcgagtttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtaccgttagccctccca

SEQ ID NO: 69 - DNA, 9414 bp, Artificial Sequence
  Synthetic Polynucleotide
attaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatgatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggccattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaattaattaagtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtcctcgagtttatggctagctcagtcctaggtacaatgctagcaattgtgagcggataacaaggctagcgaattcgagctccctctagaaataattttgtttaactttaagaaggagatataccatgggcagcagccatcatcatcatcatcacatgtcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgttcatctacatcgcgcgtctgtaccaccacgcggacccgcgtaaccgtcagggtctgcgtgacctgatctcttctggtgttaccatccagatcatgaccgaacaggaatctggttactgctggcgtaacttcgttaactactctccgtctaacgaagcgcactggccgcgttacccgcacctgtgggttcgtctgtacgttctggaactgtactgcatcatcctgggtctgccgccgtgcctgaacatcctgcgtcgtaaacagccgcagctgaccttcttcaccatcgcgctgcagtcttgccactaccagcgtctgccgccgcacatcctgtgggcgaccggtctgaaaggcggtagcggagggagtggcggtagcggagggagtgggagctcaagaggataccatatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaatctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagcaaact

SEQ ID NO: 70 - DNA, 6682 bp, Artificial Sequence
  Synthetic Polynucleotide
ggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatgatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggccattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaattaattaagtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtcctcgagtttatggctagctcagtcctaggtacaatgctagcaattgtgagcggataacaaggctagcgaattcgagctccctctagaaataattttgtttaactttaagaaggagatataccatgggcagcagccatcatcatcatcatcacatgtcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgttcatctacatcgcgcgtctgtaccaccacgcggacccgcgtaaccgtcagggtctgcgtgacctgatctcttctggtgttaccatccagatcatgaccgaacaggaatctggttactgctggcgtaacttcgttaactactctccgtctaacgaagcgcactggccgcgttacccgcacctgtgggttcgtctgtacgttctggaactgtactgcatcatcctgggtctgccgccgtgcctgaacatcctgcgtcgtaaacagccgcagctgaccttcttcaccatcgcgctgcagtcttgccactaccagcgtctgccgccgcacatcctgtgggcgaccggtctgaaataactcgagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagcaaactattaact

SEQ ID NO: 71 - DNA, 17 bp, Artificial Sequence
  Synthetic Polynucleotide
aaagtgccacctggcgg

SEQ ID NO: 72 - DNA, 36 bp, Artificial Sequence
  Synthetic Polynucleotide
gtgtatatatactcctcaaacacccttgaatctttg

SEQ ID NO: 73 - DNA, 19 bp, Artificial Sequence
  Synthetic Polynucleotide
attgacggtacgggcaacg

SEQ ID NO: 74 - DNA, 75 bp, Artificial Sequence
  Synthetic Polynucleotide
atatctccttcttaaagttaaacaaaattatttctagagggagctcgaatccctcatcagtgccaacatagtaag

SEQ ID NO: 75 - DNA, 67 bp, Artificial Sequence
  Synthetic Polynucleotide
gatacttagattcaattgtgagcggataacaatttcacacaggctagcgaccgctcattaggcgggc

SEQ ID NO: 76 - DNA, 88 bp, Artificial Sequence
  Synthetic Polynucleotide
gatacttagattcaattgtgagcggataacaatttcacacaggctagcgattaggaggaatttcaacatgacaaatttatctgacatc

SEQ ID NO: 77 - DNA, 90 bp, Artificial Sequence
  Synthetic Polynucleotide
atatctccttcttaaagttaaacaaaattatttctagagggagctcgaatcttataacattttaattttattttctccattactgtcttg

SEQ ID NO: 78 - DNA, 99 bp, Artificial Sequence
  Synthetic Polynucleotide
gccactaccagcgtctgccgccgcacatcctgtgggcgaccggtctgaaataaggagtggcggtagcggaattaaaaaagaattcaaaaaaaagcccgc

SEQ ID NO: 79 - DNA, 99 bp, Artificial Sequence
  Synthetic Polynucleotide
gttcatatggtatcctcttgagctcccactccctccgctaccgccactccttatttcagaccggtcgcgaattaaaaaagaattcaaaaaaaagcccgc

SEQ ID NO: 80 - DNA, 8401 bp, Artificial Sequence
  Synthetic Polynucleotide
acgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcctttcagcaaaaaaccccgcgagacccccgaagaggccccgcggggttatgctaggtcgacggagctcgaattctaatacgactcactatagggagaccttgacaattaatcatcggctcgtataatgccagaagcttagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaatggtttaattaacgcagcctgaatggcgaatagaagtttaaacgctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctaggaagtttaaacgctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctaggatatattgatccttgacagcttatcatcgataagctttaatgcggtagtttatcacagttgctaacgcagtcaggcaccgtgtacgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagtgatccaattcttgaag

SEQ ID NO: 81 - DNA, 8690 bp, Artificial Sequence
  Synthetic Polynucleotide
acgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcctttcagcaaaaaaccccgcgagacccccgaagaggccccgcggggttatgctaggtcgacggagctcgaattctaatacgactcactataggggcgtcgcatccggcgctagccgtaaattctatacaaaattaccgccgctccagaatctcaaagcaatagctgtgagagttctgcgcatcagcatcgtggaattcgctgaataccgattcccagtcatccggctcgtaatccgggaaatgggtgtcgccttccacttctgcgtcgatatgcgtcagatacagtttttgcgcttttggcaagaactgttcataaacgcgaccgccgccaatcaccatgatttctggtacgtcaccacacgccgcgatggcttcatccaccgacttcacccacgttacgcgatcgtccgtacccggttgactgctgaggataatatttttgcgtcctggcaacggacgaccgattgattcccaggtatggcggcccataatcacgggtttatttaaggtgttgcgtttaaaccaggcgagatcggcaggcaggttccacggcatggcgttttccatgccgataacgcgatctaccgctaacgccgcaatcagactgatcattgagatttcccgataaaaaaaattgtcgccactatacgtaaagcgtaaaccgtcgtcgactggtgcgaggatgatgttgaggaaaattttatattctgctggcgagtccacgctctctccctggactcgccgcattacaatgaaacaaaaacaaacagttagctgtaaagtgtgatttacgtcactctttattaggatgagggtttcgtttccggttcatccttaattaacgcagcctgaatggcgaatagaagtttaaacgctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctaggaagtttaaacgctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctagacctagcataaccccgcggggcctcttcgggggtctcgcggggttttttgctgaaaggctaggatatattgatccttgacagcttatcatcgataagctttaatgcggtagtttatcacagttgctaacgcagtcaggcaccgtgtacgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagtgatccaattcttgaag

SEQ ID NO: 82 - DNA, 32767 bp, Artificial Sequence
  Synthetic Polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgagacccaagctggctagttaagctatcaacaagtttgtacaaaaaagcaggctttaaaggaaccaattcagtcgactggatccggtaccaccatgttcctgaactgctgcccaggttgctgtatggagcctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggtcgactatccgtacgacgtaccagactacgcataaccgcggccgcactcgagatatctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattactttcacaggaggtacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaataggtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcgtttaagggcaccaataactgccttaaaaaaattacgccccgccctgccactcatcgcagt

SEQ ID NO: 83 - DNA, 32767 bp, Artificial Sequence
  Synthetic Polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgagacccaagctggctagttaagctatcaacaagtttgtacaaaaaagcaggctttaaaggaaccaattcagtcgactggatccggtaccaccatgttcctgaactgctgcccaggttgctgtatggagcctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggtcgactatccgtacgacgtaccagactacgcataaccgcggccgcactcgagatatctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattactaatacgactcactataggtgtggaatttcacaggaggtacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaataggtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcgtttaagggcaccaataactgccttaaaaaaatta

SEQ ID NO: 84 - DNA, 32767 bp, Artificial Sequence
  Synthetic Polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgagacccaagctggctagttaagctatcaacaagtttgtacaaaaaagcaggctttaaaggaaccaattcagtcgactggatccggtaccaccatgttcctgaactgctgcccaggttgctgtatggagcctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggtcgactatccgtacgacgtaccagactacgcataaccgcggccgcactcgagatatctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacttgacaattaatcatcggctcgtataatgatgcagtacattttcacaggaggtacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaataggtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcgtttaagggcaccaataactg

SEQ ID NO: 85 - DNA, 32767 bp, Artificial Sequence
  Synthetic Oglionucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctttaaaggaaccaattcagtcgactggatccggtaccaccatgttcctgaactgctgcccaggttgctgtatggagcctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggtcgactatccgtacgacgtaccagactacgcataaccgcggccgcactcgagatatctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattactttcacaggaggtacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaataggtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcgtttaagggcaccaataactgccttaaaaaaattacgccccg

SEQ ID NO: 86 - DNA, 32767 bp, Artificial Sequence
  Synthetic Polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctttaaaggaaccaattcagtcgactggatccggtaccaccatgttcctgaactgctgcccaggttgctgtatggagcctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggtcgactatccgtacgacgtaccagactacgcataaccgcggccgcactcgagatatctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattactaatacgactcactataggtgtggaatttcacaggaggtacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaataggtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcgtttaagggcaccaat

SEQ ID NO: 87 - DNA, 32767 bp, Artificial Sequence
  Synthetic Polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctttaaaggaaccaattcagtcgactggatccggtaccaccatgttcctgaactgctgcccaggttgctgtatggagcctgaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggtcgactatccgtacgacgtaccagactacgcataaccgcggccgcactcgagatatctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacttgacaattaatcatcggctcgtataatgatgcagtacattttcacaggaggtacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaataggtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcgt

SEQ ID NO: 88 - DNA, 1052 bp, Artificial Sequence
  Synthetic Polynucleotide
acggcgtccgcaaccggacgataatttttctgctcttcaacgaactgcgcaaaatcgtggaaacggttcgggtccagcagacgcagacgggcgaagtggctttccatccccagctgttccggggtcgcggtcagcagcagaacgcccggcacgtgctctgccagttgttcaatggcctgattcgagaaagagtgttgacttgtgagcggataacaatgatacttagattcaattgtgagcggataacaatttcacacaggctagcgaattcgagctccctctagaaataattttgtttaactttaagaaggagatataccatgggcagcagctacccatacgacgtaccagattacgcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgacttgaacgccaggcgcggcaacggggttatcaactgctgattgcctgctcagaagatcagccagacaacgaaatgcggtgcattgagcaccttttacagcgtcaggttgatgccattattgtttcgacgtcgttgcctcctgagcatcctttttatcaacgctgggctaacgacccgttcccgattgtcgcgctgg

SEQ ID NO: 89 - DNA, 3874 bp, Artificial Sequence
  Synthetic Polynucleotide
acggcgtccgcaaccggacgataatttttctgctcttcaacgaactgcgcaaaatcgtggaaacggttcgggtccagcagacgcagacgggcgaagtggctttccatccccagctgttccggggtcgcggtcagcagcagaacgcccggcacgtgctctgccagttgttcaatggcctgattcgagaaagagtgttgacttgtgagcggataacaatgatacttagattcaattgtgagcggataacaatttcacacaggctagcgaattcgagctccctctagaaataattttgtttaactttaagaaggagatataccatgggcagcagctacccatacgacgtaccagattacgcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgactctggtggttcttctggtggttctagcggcagcgagactcccgggacctcagagtccgccacacccgaaagttctggtggttcttctggtggttctagaggataccatatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaatctagagtcgacctgcaggcatgcaagcttggctgttttggcggatgagagaagattttcagttgaacgccaggcgcggcaacggggttatcaactgctgattgcctgctcagaagatcagccagacaacgaaatgcggtgcattgagcaccttttacagcgtcaggttgatgccattattgtttcgacgtcgttgcctcctgagcatcctttttatcaacgctgggctaacgacccgttcccgattgtcgcgctgg

SEQ ID NO: 90 - DNA, 3823 bp, Artificial Sequence
  Synthetic Polynucleotide
acggcgtccgcaaccggacgataatttttctgctcttcaacgaactgcgcaaaatcgtggaaacggttcgggtccagcagacgcagacgggcgaagtggctttccatccccagctgttccggggtcgcggtcagcagcagaacgcccggcacgtgctctgccagttgttcaatggcctgattcgagaaagagtgttgacttgtgagcggataacaatgatacttagattcaattgtgagcggataacaatttcacacaggctagcgaattcgagctccctctagaaataattttgtttaactttaagaaggagatataccatgggcagcagctacccatacgacgtaccagattacgcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgacggcggtagcggagggagtggcggtagcggagggagtgggagctcaagaggataccatatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaatctagagtcgacctgcaggcatgcaagcttggctgttttggcggatgagagaagattttcagttgaacgccaggcgcggcaacggggttatcaactgctgattgcctgctcagaagatcagccagacaacgaaatgcggtgcattgagcaccttttacagcgtcaggttgatgccattattgtttcgacgtcgttgcctcctgagcatcctttttatcaacgctgggctaacgacccgttcccgattgtcgcgctgg

SEQ ID NO: 91 - DNA, 8116 bp, Artificial Sequence
  Synthetic Polynucleotide
acaattaatcatcggctcgaagcttgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtagctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagcttaattaacgcagcctgaatggcgaatagggatccttgacagcttatcatcgataagctttaatgcggtagtttatcacagttgctaacgcagtcaggcaccgtgtatgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttagcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagtgatccaattcttgaagacgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcttg

SEQ ID NO: 92 - DNA, 8224 bp, Artificial Sequence
  Synthetic Polynucleotide
acaattaatcatcggctcgaagcttgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtagctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagcttaattaacgcagcctgaatggcgaatagggatccttgacagcttatcatcgataagctttaatgcggtagtttatcacagttgctaacgcagtcaggcaccgtgtatgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttagcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagtgatccaattcttgaagacgaaagggcctcgtgatacgcctatttttataggttaatgtcatgataataatggtttcttagacgtcaggtggcacttttcggggaaatgtgcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggcggccgcctttcagcaaaaaaccccgcgagacccccgaagaggccccgcggggttatgctaggtcgacggagctcgaattctaatacgactcactatagggagacccaagctggcttg

SEQ ID NO: 93 - DNA, 72 bp, Artificial Sequence
  Synthetic Polynucleotide
aaagagtgttgacttgtgagcggataacaatgatacttagattcaattgtgagcggataacaatttcacaca

SEQ ID NO: 94 - DNA, 76 bp, Artificial Sequence
  Synthetic Polynucleotide
gacaccatcgaatggcgcaaaacctttcgcggtatggcatgatagcgcccggaagagagtcaattcagggtggtga

SEQ ID NO: 95 - DNA, 29 bp, Artificial Sequence
  Synthetic Polynucleotide
ttgacaattaatcatcggctcgtataatg

SEQ ID NO: 96 - DNA, 76 bp, Artificial Sequence
  Synthetic Polynucleotide
gacaccatcgaatgttgacaattaatcatcggctcgtataatgagcgcccggaagagagtcaattcagggtggtga

SEQ ID NO: 97 - protein, 229 aa, Artificial Sequence [sequence omitted]

SEQ ID NO: 98 - protein, 145 aa, Artifical Sequence
  Synthetic Polypeptide
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKAPV

SEQ ID NO: 99 - protein, 6 aa, Artifical Sequence
  Synthetic Polypeptide
GGSGGS

SEQ ID NO: 100 - protein, 12 aa, Artificial Sequence
  Synthetic Polypeptide
GGSGGSGGSGGS

SEQ ID NO: 101 - protein, 65 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGS